(19) United States
(12) Patent Application Publication
(10) Pub. No.: US 2017/0007561 A1
(43) Pub. Date: Jan. 12, 2017

(54) CONTEMPORANEOUS, HETEROGENEOUSLY-ORIENTED, MULTI-TARGETED THERAPEUTIC MODIFICATION AND/OR MODULATION OF DISEASE BY ADMINISTRATION OF SULFUR-CONTAINING, AMINO ACID-SPECIFIC SMALL MOLECULES

(71) Applicant: Frederick H. Hausheer, Fair Oaks Ranch, TX (US)

(72) Inventor: Frederick H. Hausheer, Fair Oaks Ranch, TX (US)

(73) Assignee: BioNumerik Pharmaceuticals, Inc.

(21) Appl. No.: 14/455,855

(22) Filed: Aug. 8, 2014

(51) Int. Cl. A61K 31/185; A61K 31/517; A61K 31/198; C07K 5/062; C07K 5/083; C12Q 1/34; A61K 38/06; A61K 45/06; C12Q 1/48; C12Q 1/26; G01N 33/50; C12Q 1/28; A61K 31/4545; A61K 38/05

(52) U.S. Cl. CPC A61K 31/185; A61K 31/4545; A61K 31/517; A61K 31/198; C07K 5/0606; C07K 5/081; A61K 38/05; A61K 38/06; A61K 45/06; C12Q 1/48; C12Q 1/26; G01N 33/5023; C12Q 1/28; C12Q 1/485; C12Q 1/34; G01N 2333/912; G01N 2500/04; G01N 2500/20; G01N 2333/908; G01N 2333/922; G01N 2333/91171

(57) ABSTRACT

The present invention discloses and claims novel pharmaceutical compositions, methods, and kits used for the contemporaneous, heterogeneously-oriented, multi-targeted therapeutic modification and/or modulation of cellular metabolic anomalies or other undesirable physiological conditions, including cancer, where the normal cellular biochemical function and/or the expression levels of various proteins/enzymes (i.e., the target molecules) are abnormal and must be modified and/or modulated in order to treat these metabolic anomalies or other undesirable physiological conditions, including cancer. The aforementioned target molecules, by way of non-limiting example, include: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and various other classes of proteins/enzymes. Additionally, the present invention discloses and claims methods and kits for (a) the selection of subjects for treatment; (b) the determination of the most effective medicinal agent(s) to be administered in combination with the administration of the sulfur-containing, amino acid-specific small molecules of the present invention; (c) the dosage of the medicinal agent(s) to be administered; (d) the determination of the length and/or number of treatment cycles; (e) the adjustment of the specific medicinal agent(s) used and the dosage administered during treatment; and/or (f) ascertaining the potential treatment responsiveness of the specific disease to the medicinal agents (s) selected for administration to a subject suffering from one or more types of: (i) cancer or (ii) metabolic anomalies or other undesirable physiological conditions by quantitatively determining the level of the abnormal biochemical activity and/or abnormal expression of any combination of the aforementioned target molecules; by use of quantitative measurement methodologies including, but not limited to: fluorescence in situ hybridization (FISH), nucleic acid microarray analysis, immunohistochemistry (IHC), radioimmunoassay (RIA), quantitative immunofluorescence and/or automated quantitative analysis; ELISA and flow cytometry-based analyses; PCR coupled with MS approaches; mass spectroscopy-based methods; and X-ray crystallography, and other related analytic methodologies.

### RELATED APPLICATIONS

The present application claims priority to U.S. Provisional Patent Application Ser. No. 61/865,360, with a filing date of Aug. 13, 2013, and entitled: “CONTEMPORANEOUS, MULTIPLE PROTEIN-TARGETED THERAPEUTIC MODIFICATION AND/OR MODULATION OF DISEASE BY ADMINISTRATION OF SULFUR-CONTAINING, AMINO ACID-SPECIFIC SMALL MOLECULES”, the disclosure of which is herein incorporated by reference in its entirety.

### FIELD OF THE INVENTION

The present invention relates to novel pharmaceutical compositions, methods, and kits used for the treatment of cancer and other medical conditions. More specifically, the present invention relates to novel pharmaceutical compositions, methods, and kits comprising medicaments used for the treatment of cellular metabolic anomalies such as cancer or other undesirable physiological conditions where the normal cellular biochemical function and/or the expression levels of various proteins (i.e., target molecules of the present invention) are abnormal and must be modified and/or modulated in order to treat these metabolic anomalies. The aforementioned target molecules, by way of non-limiting example, include: protein tyrosine kinases, DNA synthesis and repair proteins, structural proteins, oxidoreductases, and various other classes of proteins/enzymes. Additionally, the present invention discloses and claims methods and kits for (a) the contemporaneous modification/modulation of multiple target molecules; (b) the treatment of cancer and other undesirable physiological conditions; (c) the selection of subjects for treatment; (d) the determination of the most effective medicinal agent(s) to be administered in combination with the administration of the sulfur-containing, amino acid-specific small molecules of the present invention; (e) the dosage of the medicinal agent(s) to be administered; (f) the determination of the length and/or number of treatment cycles; (g) the adjustment of the specific medicinal agent(s) used and the dosage administered during treatment; and/or (h) ascertaining the potential treatment responsiveness of the specific abnormal metabolic condition to the medicinal agent(s) selected for administration to a subject suffering from one or more types of cancer or other undesirable physiological conditions by quantitatively determining: (i) the abnormal biochemical activity and/or (ii) the level of abnormal expression of any combination of the target molecules of the present invention.

### BACKGROUND OF THE INVENTION

### I. Brief Overview of Present Invention

Large numbers of current approaches to the treatment of cancer and many other diseases have been focused on identifying a single genetic or molecular target of interest, and then developing therapies to interact with the identified target in order to treat the disease. An example of this focus is the growing trend in oncology to develop “personalized therapies” aimed at addressing a particular genetic mutation in an identified portion of the cancer population.

While many of these approaches can provide some benefit to patients, they are only a first step towards achieving comprehensive and lasting treatment benefits. This is due to the heterogeneous nature of cancer and many other diseases. Because cancer is heterogeneous, single-targeted approaches frequently leave other cancer-implicated targets and pathways unaddressed, allowing the underlying disease to progress.

Given the limitations of these current approaches, a treatment that was able to contemporaneously interact with multiple target molecules of interest would be beneficial and would represent a next step in treating cancer and many other diseases.

The teachings in the present application take into account the concept of disease heterogeneity, in combination with new observations and data, in order to provide novel methods, pharmaceutical compositions, and kits used for the treatment of cancer and other medical conditions.

Unlike the current trend, which utilizes single molecular-targeted treatment approaches, the present invention discloses methods and compositions to contemporaneously modulate and interact with multiple target molecules in order to provide treatment for a variety of cellular metabolic anomalies or other undesirable physiological conditions.

As will be discussed in greater depth infra, it has recently become increasingly apparent that cancer is a heterogeneous disease and involves the complex interaction of numerous genes, enzymes/proteins, and metabolic pathways. Accordingly, as cancer progression becomes characterized as a genome-mediated macro-evolution (rather than a gene-centric developmental process), a change of research and drug development strategy is required in order to more fully treat the disease.

In contrast to the gene-centric concept of cancer, according to the genome-centric concept of cancer evolution (see, e.g., Ye, C. J., Stevens, J. B., et al. Genome based cell population heterogeneity promotes tumorigenicity: the evolutionary mechanism of cancer. J. Cell Physiol. 219:288-300 (2009)) the key to understanding cancer is to not focus on specific genetic or epigenetic alterations, but rather to study the evolutionary mechanisms of cancer to effectively address the issue of genome system heterogeneity. First, cancer progression is an evolutionary process where genome system replacement (rather than a common genetic/epigenetic pathway) is the driving force. Second, there are potentially unlimited numbers of genetic and epigenetic alternatives along with all types of environmental stress that can contribute to the cancer evolution and it is highly unlikely that one could identify a universal molecular mechanism. See, e.g., Heng, H. H., Stevens, J. B., et al. Patterns of genome dynamics and cancer evolution. Cell. Oncol. 30:513-514 (2008). Further, heterogeneity is a key mate feature of cancer and it is extremely difficult, if not impossible, to apply these diverse genetic/epigenetic patterns to clinical methodologies that require precise modes of prediction. Therefore, the true challenge lies in understanding the genetic/epigenetic system behavior (i.e., their stability or instability) and the unpredictable replacement and switching that occurs between various pathways during cancer progression and, in particular, during subsequent medical intervention.

Heavily influenced by reductionism's view, the majority of the current molecular analyses of cancer have been focused upon a single molecule of interest, without considering other molecules and the overall status of the biological/genomic systems in toto. It has also been generally assumed during molecular manipulation or specific targeting, that the biological system remains unperturbed. This assumption has been pushed to the extreme, where genome level information has become largely ignored by most of the molecular analyses.

While there have been some advances made in the treatment of cancer by the development and administration of, e.g., monoclonal antibodies, unfortunately, most of these agents are specific only for a single protein/enzyme target. In contrast, the sulfur-containing, amino acid-specific small molecules of the present invention possess the ability to contemporaneously or simultaneously modify and/or modulate: (i) the abnormal biochemical activity and/or (ii) the abnormal expression of a multiple number of enzymes/proteins (i.e., target molecules). These target molecules include, but are not limited to, anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif.

In brief, abnormal expression (or increased catalytic activity, or both) of certain proteins/enzymes mediates a multi-component, multi-pathway mechanism which confers a survival advantage to cancer cells. This abnormal expression/increased levels in cancer cells can lead to several important biological alterations including, but not limited to: (i) loss of apoptotic sensitivity to therapy (i.e., drug or ionizing radiation resistance); (ii) increased conversion of RNA into DNA (involving ribonucleotide reductase); (iii) altered gene expression; (iv) increased cellular proliferation signals and rates; and/or (v) increased angiogenic activity (i.e., increased blood supply to the tumor).

Accordingly, contemporaneous or simultaneous modification and/or modulation of the aforementioned target molecules by the administration of effective levels and schedules of the sulfur-containing, amino acid-specific small molecules of the present invention can result in substantial improvements in the effects of cancer treating agents along with substantially improved outcomes for patients.

In addition, there are a number on non-cancer-related metabolic anomalies or other undesirable physiological conditions that exhibit evidence of: (i) the abnormal biochemical activity and/or (ii) the abnormal expression of the same aforementioned target molecules. These metabolic anomalies or other undesirable physiological conditions include, but are not limited to, heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigementosum, Cockayne syndrome, Fanconi anemia, and cerebro-oculo-facio-skeletal syndrome. Accordingly, the administration of the sulfur-containing, amino acid-specific small molecules of the present invention can also be effective in the treatment of these metabolic anomalies or other undesirable physiological conditions.

The sulfur-containing, amino acid-specific small molecules of the present invention may be administered using any combination of the following three general treatment methods in order to attain the full benefit of their contemporaneous and multi-targeted characteristics: (i) in a direct inhibitory or inactivating manner (i.e., direct chemical interactions) by, e.g., Tavocept-mediated xenobiotic modification of Cys residues that inactivates one or more of the aforementioned target molecules and/or a direct depletive manner (i.e., decreasing target molecule concentrations or production rates), thereby increasing the susceptibility of the cancer cells to any subsequent administration of any cancer treating agent or agents that may act directly or indirectly through the target molecule-mediated pathways in order to sensitize the patient's cancer and thus increase the survival of the patient; (ii) in a synergistic manner, where the target molecule-specific therapy is concurrently administered with chemotherapy administration when a cancer patient begins any chemotherapy cycle, in order to increase and optimize the pharmacological activity directed against target molecule-mediated mechanisms present while chemotherapy is being concurrently administered; and/or (iii) in a post-treatment manner (i.e., after the completion of chemotherapy dose administration or a chemotherapy cycle) in order to maintain the presence of a pharmacologically-induced depletion, inactivation, or modulation of one or more of the target molecules in the patient's cancer cells for as long as optimally required.

### II. The Heterogeneous Nature of Cancer

Cancer is a highly complex, diverse disease that is heterogeneous, rather than homogeneous. The fact that some specific types of cancer are microscopically heterogeneous has been known for over a century; whereas chromosomal and molecular heterogeneity was subsequently discovered as a direct result of the more recent technological advances in cellular and molecular biology. Further studies have shown that although cancer in a given individual may start in a clonal manner, subsequent mutations frequently occur due to the “pressures” exerted upon said cancer by the use of therapy (e.g., chemotherapy, radiation, and the like) and/or various other evolutionary factors that can lead to metastases or therapeutic resistance.

By way of example, a recent large clinical study (see, Gerlinger, M., et al., Intratumor Heterogeneity and Branched Evolution Revealed by Multiregion Sequencing: New Engl. J. Med. 366:883-892 (2012)) has demonstrated how cancer in any one patient (and even within the same tumor) can be far more heterogeneous than ever initially believed. In this study, the authors evaluated multiple tumor biopsy samples from a variety of primary and metastatic sites in patients with metastatic renal cell carcinoma. Subsequent extremely detailed cytogenetic and genetic analyses allowed the authors to reconstruct the evolutionary growth of tumors in each of the samples. One of the primary focuses of the study was to take multiple biopsy samples within the same tumor, with some samples being less than a centimeter apart.

A synopsis of the aforementioned study's findings include: (i) phylogenetic reconstruction revealed branched evolutionary tumor growth, with 63-69% of all somatic mutations not being detectable across every region of the tumor; (ii) intratumor heterogeneity was observed for a mutation within an autoinhibitory domain of the mammalian target of rapamycin (mTOR) kinase, correlating other molecular control points (e.g., other kinase activity); (iii) mutational intratumor heterogeneity was seen for multiple tumor suppressor genes converging on loss of function with multiple distinct and spatially separated inactivating mutations within a single tumor, suggesting convergent phenotypic tumor evolution; (iv) gene expression signatures of both good and poor prognosis were detected in different regions of the same tumor; and (v) allelic composition and ploidy profiling analysis revealed extensive intratumor heterogeneity, with 26 of 30 tumor samples from four different tumors all harboring divergent allelic-imbalance profiles (if this proves to be a general finding for other cancers, the effects on diagnostic confidence would be profound, as the usual diagnostic methodology involves a needle biopsy of the accessible tumor with the sample subdivided for various diagnostic tests, including some types of molecular analysis).

The current trend in oncology to provide, if feasible, “personalized therapy” in which selection of the specific therapeutic approache(s) utilized is based upon a positive historical response in a patient with the same genomic or cytogenetic profile. Thus, this “personalized” approach depends on identifying an appropriate genomic or cytogenetic profile. However, if even a small number of integral genomic determinants are as variable as those demonstrated in the aforementioned Gerlinger study (Gerlinger, M., et al., Intratumor Heterogeneity and Branched Evolution Revealed by Multiregion Sequencing: New Engl. J. Med. 366:883-892 (2012)), such personalized therapy becomes exponentially more difficult. The Gerlinger study also found that heterogeneity also applied to favorable as well as unfavorable prognostic features. Intra-tumor heterogeneity can lead to underestimation of the overall tumor genomic profile portrayed from single tumor-biopsy samples and may present major challenges to personalized-medicine and biomarker development. Therefore, intra-tumor heterogeneity, associated with heterogeneous protein function, may explain the difficulties encountered in the validation of oncology biomarkers owing to sampling bias, and also contribute to Darwinian selection of preexisting drug-resistant clones, thereby resulting in therapeutic resistance. Moreover, if this degree of heterogeneity is found in many (or all) common tumors, clinical trials targeted to specific genomic profiles/mutations will become more difficult or even impossible to complete using modern molecular diagnostics with current trial methodologies; thus stressing the importance of combination therapy or multi-targeted agents being considered earlier in the drug development process.

### Gene-Centric Versus Genome-Centric Concept of Cancer

Traditional strategies in cancer research have been focused on the identification and characterization of the general patterns of genetic aberrations and in particular, key “cancer gene” mutations. The underlying principle of this paradigm has been that specific types of cancer are caused by sequential genetic events occurring during “cancer development.” This gene-centric view has dominated the field of cancer research for decades resulting in the concentration of research efforts on defining mutated oncogenes, tumor suppressor genes, and their molecular pathways. See, e.g., Heng, H. H. Cancer genome sequencing: the challenges ahead. BioEssays 29:783-794 (2007). Despite the initial success of identifying a number of gene mutations that had a high percentage among certain patient populations, most subsequently identified gene mutations have displayed low frequencies among patients. Moreover, the list of cancer genes continues to grow, which brings into question the goals and rationale of continuing to attempt to identify a handful of commonly shared gene mutations in cancer. Thus, it is clear that the current concept of cancer is not consistent with the reality of the presence of high degrees of genetic diversity in patients. In an attempt to solve this dilemma, cancer genome sequencing has been proposed to identify these common cancer genes, based on the assumption that cancer heterogeneity among patients is genetic “noise” and can be eliminated by validation using large patient samples. Unfortunately, this approach is delivering unwanted, conflicting results and it has not been successful to date. See, e.g., Greenman, C., Stephens, P., et al. Patterns of somatic mutation in human cancer genome. Nature 446:153-158 (2007).

In another example of heterogeneity, research has also shown that the vast majority of gene mutations are not uniformly shared among patients. In view of this finding, many researchers are trying to decide what avenue(s) to subsequently explore. By way of example, some have suggested shifting from gene identification to pathway characterization (see, e.g., Jones, S., Zhang, X., et al. Core signaling pathways in human pancreatic cancers revealed by global genomic analyses. Science 321:1801-1806 (2008)), others are searching for non-gene-related causes including, but not limited to: bacterial/viral infections, cancer stem cells, metabolic stress and errors, oxidative stress, aneuploidy, inflammation, tumor/tissue interaction, immuno-deficiency, a large array of epigenetic effects, and non-coding RNAs. If one examines the underlying motif, these aforementioned approaches represent the same attempt to find common causative patterns which are now focused on different levels of genetic/epigenetic or cellular organization and their response under various types of environmental stress.

In contrast to the gene-centric concept of cancer, the position advanced by the genome-centric concept of cancer is that the key to understanding cancer is to not focus on specific genetic or epigenetic alterations, but rather to study the evolutionary mechanisms of cancer and to effectively address the issue of genome system heterogeneity. See, e.g., Ye, C. J., Stevens, J. B., et al. Genome based cell population heterogeneity promotes tumorigenicity: the evolutionary mechanism of cancer. J. Cell Physiol. 219:288-300 (2009). First, cancer progression is an evolutionary process where genome system replacement (rather than a common pathway) is the driving force. Second, there are potentially unlimited numbers of genetic and epigenetic alternatives along with all types of environmental stresses that can contribute to the cancer evolution and it is highly unlikely that one could identify a universal molecular mechanism. See, e.g., Heng, H. H., Stevens, J. B., et al. Patterns of genome dynamics and cancer evolution. Cell. Oncol. 30:513-514 (2008). Further, heterogeneity is a key inate feature of cancer and it is not practical to apply diverse genetic/epigenetic patterns to clinical usage that requires precise prediction. Therefore, the true challenge lies in understanding the overall system behavior (i.e., stability or instability) and the unpredictable replacement and switching that occurs between various pathways during cancer progression and, in particular, during subsequent medical intervention. Thus, there remains the need to elecudiate how the cellular system heterogeneity plays a role in cancer evolution. Detailed information on current epigenetic research can be found in numerous reviews. See, e.g., Mohn, F., Schubeler, D. Genetics and epigenetics: stability and plasticity during cellular differentiation. TIGS 25:129-136 (2009).

### Recent View and Research on Cancer Heterogeneity

Early efforts in the study of cancer heterogeneity focused on morphological and pathological heterogeneity. Prior to the molecular biological/oncogene era, multiple levels of heterogeneity were described including: cellular morphology; tumor histology; karyotype and other cytogenetic markers; growth rate; cell products; receptors; enzymes; immunological characteristics; metastatic ability; and sensitivity to therapeutic agents. Unfortunately, however, much of this important information has been ignored by many molecular geneticists, who tend to focus only upon the identification of common patterns of gene mutations. The limitation of molecular methodologies also contributes to this oversight. These limitations include extensive analysis of specific genes or pathways without monitoring of the entire system. Specifically, the heterogeneity of the dynamic cellular genome has been left out. In addition, methodologies of DNA/RNA isolation and sequencing from mixed cell populations artificially average the molecular profile (see, e.g., Bielas, J. H., Loeb, K. R., et al. Human cancers express a mutator phenotype. Proc. Natl. Acad. Sci. USA 103:18238-18242 (2006)) which favors the identification of aggressive and/or well studied pathways by the “washing away” of any heterogeneity that is present. This gives a false impression that the accumulation of “clonal” cancer gene mutations or epigenetic changes represents a significant pattern in most cancers (which is not the case). Despite this inate experimental bias for the mitigation/elimination of heterogeneity, high levels of heterogeneity are still overwhelmingly detected. Table 1, below, summarizes examples of some of the key features of heterogeneity that exist at multiple genetic and epigenetic levels. It should be noted that the list of elements enumerated in Table 1 that contribute to system heterogeneity is growing rapidly, and even more importantly, each element has the ability to interact with other elements, thus forming an almost unlimited combinational heterogeneity.

TABLE 1
Multiple Levels of Genetic/Epigenetic/Environmental Heterogeneity

Multiple genetic levels:
Gene/nucleotide level:
Nucleotide polymorphism
Various types of repeats (e.g., microsatellite shifts)
Spectrum of mutations (including conditional mutations)
Heterozygosity (allelic)
Splicing forms
Gene family members (paralogs)
Combinational effects of multiple genes and mutations
Genome/chromosome and sub-chromosome level:
Copy number variation, microdeletion/inversion
Loss of heterozygosity (LOH)
Chromosomal translocation/inversion/duplication
Defective mitotic figures
Chromosome fragmentation
Aneuploidy
Polyploidy
Multiple epigenetic levels:
Chromatin folding and attachment to the nuclear matrix
Packaging of nucleosomes
Position of histone variants
Covalent modification of histone tails
DNA methylation
Non-coding RNAs
Change of system status independent of epigenetic alteration
Environmental influence on the multiple levels of homeostasis:
Tissue specificity
Physiological condition alteration (aging, immune, hormone,
and metabolic levels)
Nutrition status
Different types of exposure stress
Variety on dosage, duration of the exposure
Differential impact on individual cell/organs
Certain levels of stochastic response

Even though there is increasing documentation of the high level of heterogeneity factors in cancer, their biological significance has been less clear from a molecular aspect and the methods to study them have been lacking. Recently however, there is a trend to re-investigate the issue of cancer genetic and epigenetic heterogeneity due to the following developments:
(i) It has been difficult to identify common patterns even when utilizing the most advanced molecular biological methodologies (e.g., whole genome sequencing and expression arrays). This intense research effort often reveals increased heterogeneity rather than a specific pattern. For example, p53 is one of the most studied molecules and has the highest level of heterogeneity. See, e.g., Whibley, C., Pharoah, P. D., Hollstein, M. p53 polymorphisms: cancer implications. Nat. Rev. Cancer 9:95-107 (2009).
(ii) Increased usage of molecular cytogenetic methods (e.g., FISH) when coupled with laser micro-dissection can differentiate profiles of individual cells from the same tumor or can compare primary and metastatic tumors from the same patient. See, e.g., Bayani, J., Selvarajah, S., et al. Genomic mechanisms and measurement of structural and numerical instability in cancer cells. Semin. Cancer Biol. 17:5-18 (2007). This type of analysis has clearly demonstrated that genome level alteration within tumors is a universal feature.
(iii) The debate between the cancer stem cell model and the clonal evolution model has caused researchers to rethink the issue of heterogeneity, as tumor heterogeneity represents a key feature of the cancer evolution model. See, e.g., Campbell, L, Polyak, K. Breast tumor heterogeneity: cancer stem cells or clonal evolution? Cell Cycle 6:2332-2338 (2007).
(iv) Growing research underscores the importance of epigenetic regulation in cancer. In particular, the behavior of nuclear structure and chromatin domains and non-coding RNA has gained more attention. See, e.g., Ventura, A., Jacks, T. MicroRNAs and cancer: shortRNAs go a longway. Cell 136:586-591 (2009). Different from gene mutations, multiple levels of regulators are more dynamic with less specificity (i.e., higher heterogeneity) when regulating genes.
(v) It has become clear that an improved theoretical framework for cancer research is now urgently needed and the concept of somatic evolution represents just such a framework. See, e.g., Heng, H. H. The genome-centric concept: re-synthesis of evolutionary theory. BioEssays 31:512-525 (2009).

### Understanding Cancer Heterogeneity

Understanding the importance of heterogeneity is the key to understanding the general evolutionary mechanism of cancer. Unfortunately, there are many inaccurate preconceived assumptions regarding cancer heterogeneity which have impeded progress in this field. Classic physical sciences have influenced the debate between random genomic background “noise” and heterogeneity where it is thought that the quantity being measured is unchanged and variability results only from random measurement errors. Performing additional measurements is often used to solve variance issues. However, bio-variation is a very unique intrinsic feature of biological systems. Thus, simply increasing sample size will not solve the heterogeneity issue, as heterogeneity is not random noise and should not simply be disregarded. See, e.g., Heng, H. H. The conflict between complex systems and reductionism. JAMA 300:1580-1581 (2008). In fact, the heterogeneity “noise” represents a key feature of biological systems, providing needed complexity and robustness. As for any given biological system, the patterns detected are defined by the genome context and are environmentally-dependent. See, e.g., Heng, H. H. The genome-centric concept: re-synthesis of evolutionary theory. BioEssays 31:512-525 (2009). By changing the environment, a specific pattern could thus become more sporadic or “noise-like” or not essential to a given process (and vice versa). Accordingly, the exsistance of heterogeneity provides a greater chance of success that a system will be capable of adapting to the given environment and survive.

Many researchers now postulate that heterogeneity is the reason that universal mutations can not be identified. This is illustrated by the finding that, in a majority of cases of the same type of cancer, most patients display a unique array of mutations that only have minimal inter-patient homogeneity. In a highly dynamic complex biological system, such as cancer, any given pattern might represent only a limited number of cases, as cancer cases are genetically- and environmentally-contingent. The pattern of specific gene mutations can only be used within a specific population with a similar genome, mutational composition, and a similar environment. In addition, it is difficult to identify and even more difficult to apply specific patterns of gene mutations to the treatment of solid tumors, as the most common feature in tumors is a high level of genome variation which often changes the function of a specific gene mutation. There is a need to change the way of thinking—by focusing more on monitoring the level of heterogeneity, rather than attempting to identify specific patterns.

Therefore, in conclusion, the frequently utilized strategy of attempting to reduce heterogeneity in order to study the mechanisms of cancer represents a flawed approach. Importantly, many researchers in the field currently believe that without heterogeneity, there would be no cancer. That is the reason why many principles discovered using simplified homogenous experimental systems do not apply in the real world of cancer-related heterogeneity.

### Stepwise Cancer Development Versus Stochastic Macro-Evolution

As judged by drastically different karyotypes and gene mutation profiles, each tumor seems to represent one independent run of somatic evolution and does not follow a stepwise reproducible pattern. This situation is different from the natural evolution (comprised of only one run of evolution) that is familiar in non-neoplastic tissues. When tracing natural evolution, many key genes can be traced from model organisms. However, this clearly does not apply to the majority of cancers, as it would be difficult to trace the same gene mutations or ultraconserved regions among cases that evolved during different runs of evolution. See, e.g., Heng, H. H. The genome-centric concept: re-synthesis of evolutionary theory. BioEssays 31:512-525 (2009); Mohn, F., Schubeler, D. Genetics and epigenetics: stability and plasticity during cellular differentiation. TIGS 25:129-136 (2009). Examples include the fact that most of the karyotypes of solid tumors are drastically altered compared with the normal human; there is a significant correlation between karyotype heterogeneity and poor prognosis; and the recent finding that some regions of the genome are conserved by organismal evolution but altered in cancers. See, e.g., Calin, G. A., Liu, C. G., et al. Ultraconserved regions encoding ncRNAs are altered in human leukemias and carcinomas. Cancer Cell 12:215-229 (2007). In addition, there are many sub-types of the same cancer, and it is possible that the same tumor can evolve from multiple cell lineages. Moreover, it has recently been demonstrated that even a single cell can generate cells with drastically different karyotypes as this stochastic process generates heterogeneity. It should be noted, that the stochastic events referred to herein are not completely random; but rather are less predictable due to differences in the initial conditions reflected by the multiple levels of genetic and epigenetic alteration.

Due to the same reason as in stochastic macro-evolution, cancer progression is fundamentally different from developmental processes. Developmental progression refers to the well controlled process of self-organization (both temporally and spatially) where many key genes play a crucial role; whereas in cancer evolution, even though some cases involve parts of the developmental process, in a majority of cancer cases, the dominant alterations are genome mediated stochastic system replacement, which does not follow a well controlled pattern. Accordingly, researchers argue that the terminology “cancer development” implies an incorrect concept and needs to be altered.

### Gene Function Within Different Systems

Since cancer progression is characterized as genome mediated macro-evolution (rather than micro-evolution or a developmental process), it requires a change of research strategy. Heavily influenced by reductionism's view, most of the molecular analyses of cancer have been focused on a particular molecule of interest, without considering the overall status of the genome system. It has been generally assumed during molecular manipulation or specific targeting that the biological system remains the same. For many exsisting approaches, this assumption has been pushed to the extreme, where genome level information has become largely ignored by most of the molecular analyses. However, this is an erroneous assumption, as when the overall karyotype changes, the role of the same gene may also be altered, as the function of genes are dependent upon their genetic network which is defined by the genome context. See, e.g., Heng, H. H. The genome-centric concept: re-synthesis of evolutionary theory. BioEssays 31:512-525 (2009). This is even more true in cancer research, as the systems continually change during progression of the disease, as is illustrated by significantly altered karyotypes and gene expression patterns. See, e.g., Heng, H. H. The conflict between complex system and reductionism. JAMA 300:1580-1581 (2008).

Unfortunately, however, few studies have been performed to analyze the biological meaning of drastic genome level alterations, which in fact could explain many contradictory findings occurring at the pathway level when cells with different karyotypes are analyzed. By way of example, the p53 pathway has been linked to diverse molecular mechanisms or pathways and at least 50 different enzymes can covalently modify p53 to alter its function and several thousand genes have been shown to be directly regulated by p53. See, e.g., Kruse, J., Gu, W. SnapShot: p53 posttranslational modifications. Cell 133:930-930 (2008). Interestingly, each of these characterized functions represents one possible potential function defined by the genome context, including epigenetic regulation of the same genome but different tissue type. See, e.g., Murray-Zmijewski, F., Slee, E. A., Lu, X. A complex barcode underlies the heterogeneous response of p53 to stress. Nat. Rev. Mol. Cell. Biol. 9:702-712 (2008). Clearly, for a given cell, most of the known mechanisms of p53 mutation cannot simultaneously function. One of the reasons the functional list of p53 mutations keeps growing is this molecule has been extensively examined using drastically different genome systems. As the majority of different cell lines and tumor samples that have been used in different experiments display different karyotypes, the large array of different p53 functions and its pattern within disease network, reflect the possibilities of functions created through system heterogeneity (in addition to the network complexity of a given system). Genome level heterogeneity also makes the function of a p53 mutation more visible and versatile (and thus more important) than it should be, as each of the individual functions can be stochastically selected in a population with heterogeneity.

Thus, the question remains—which level of the biological system needs to become the primary focus? It is generally accepted that any biological system can be classified into different levels and it is up to the individual researcher to choose the proper level of analysis based upon the available concepts and methodologies. According to the concept of complexity, the approach and rationale of reducing complexity to the lowest level usually does not work, as emergent properties of lower level parts are often very different from the overall system. Equally important is that information theory suggests that the selection of the level that controls a system is very crucial and, in contrast, the level that is easiest to access information from is usually not very useful in the control of a given system. Thus, understanding how complexity and information theories apply to biological systems will greatly influence research strategies.

### The Causative Relationship Between Heterogeneity and Cancer Progression

The aforementioned issues discussed above lead to an important question—During stochastic cancer evolution, where there are seemingly unlimited contributing factors, is it possible to establish a causative relationship among specific gene mutations/epigenetic alterations and cancer progression within the background of multiple levels of genetic/epigenetic and environmental heterogeneity? The establishment of such a causative relationship has been the goal of cancer molecular biology. To establish such a relationship, one needs to identify a pattern of consistent response and events of cause-and-effect by fixing the initial conditions within this dynamic network. Thus, by fixing the initial conditions, the ability to define the causative relationship in a linear reaction or pathway as the cause-and-effect is more easily established and more meaningful. Many examples have been illustrated from developmental studies where many causative relationships between genes and morphological features have been elegantly documented. In cases of complex systems with high levels of heterogeneity (e.g., pathological conditions that are often stochastically caused by less controlled factors), there seems to be no clear cut causative relationship. This is particularly true when studying the highly dynamic genetic network of cancer. Not only can the dynamics of the same system change, but the systems can keep changing during cancer evolution. Due to non-linear relationships and many stochastic forward and feedback loops, any particular cause will have drastically different effects depending on system dynamics coupled with varying environmental effects. In addition, the same cause can have drastically different effects depending on genetic and epigenetic heterogeneity; whereas, conversely, different causes can have similar effects in terms of system behavior.

In sum, is very challenging to identify the main response of a given pathway, particularly in cancer cells where the genome context is constantly changing. Further, different cancer cells within the same tumor often respond to the same treatment differently through the activation of drastically different pathways. This ability is a key advantage of heterogeneity from the perspective of tumor cell survival and evolution. By ignoring the overall system and its complexity, it is easier to identify an order among parts; however, this information is highly likely to not represent the actual situation of emergent properties at the higher level of a system. Based upon this thinking, many scientists in this field recommend focusing upon correlation studies rather than search for a specific “causal relationship”. See, e.g., Ye, C. J., Stevens, J. B., et al. Genome based cell population heterogeneity promotes tumorigenicity: The evolutionary mechanism of cancer. J. Cell Physiol. 219:288-300 (2009).

### The Genome-Centric Concept of Cancer Evolution

In the genome-centric concept of cancer evolution, genome-level heterogeneity is a primary factor in cancer evolution in the vast majority of cancers. Empirically, it seems rather complicated to deal with the issue of heterogeneity, as many levels and various types of heterogeneity are involved (see, Table 1) and the selection of a dominant pathway occurs stochastically. Paradoxically, despite the difficulties in establishing a causative relationship among individual molecular mechanisms within a complex biological system, it is relatively easy to establish a causative relationship between system heterogeneity and cancer evolution, as heterogeneity is the necessary pre-condition needed for cancer evolution to occur. The degree of this heterogeneity can be quantitatively measured. See, e.g., Heng, H. H. The genome-centric concept: re-synthesis of evolutionary theory. BioEssays 31:512-525 (2009); Ye, C. J., Stevens, J. B., et al. Genome based cell population heterogeneity promotes tumorigenicity: The evolutionary mechanism of cancer. J. Cell Physiol. 219:288-300 (2009); Heng, H. H. The conflict between complex system and reductionism. JAMA 300:1580-1581 (2008). By way of example, if one focuses on the overall level of system dynamics (and if this is the principal level of selection during cancer evolution), then it is possible that one can monitor the overall pattern of heterogeneity at the principal level of evolution.

Considering the fact that regardless of which specific factor(s) induce system instability, increased levels of system dynamics can be measured using pattern changes such as increased “system randomness”. Thus, it would be more useful to study system behavior by monitoring the overall heterogeneity status rather than monitoring specific pathways, as it would be difficult to predict system evolution based upon a specific pathway, particularly when pathways have low patient population penetration and low predictability.

Numerous researchers have demonstrated the importance of using non-specific changes at the genome level to monitor genetic heterogeneity and its crucial role in cancer evolution, as the increased probability of cancer evolution becomes far more important than any specific pathway. Specifically, this research has defined cancer progression as macro-evolution where the major underlying force is karyotypic heterogeneity even though this process is associated with large numbers of seemingly random gene mutations and epigenetic alterations. Only within relatively stable stages (i.e., where there is no karyotypic change), do gene mutations and epigenetic regulation play a dominant role, similar to the adaptation phase of micro-evolution. In order to quantitate this difference, monitoring heterogeneity at different levels of genetic organization is required. In contrast, to detect internal system modifications, monitoring gene mutations and epigenetic changes is more appropriate. From a system point of view, significant karyotypic changes represent a “point of no return” in biological system evolution, even though certain gene mutations and epigenetic changes can influence karyotypic changes.

### Non-Clonal Chromosome Aberrations (NCCAs) are an Important Indicator of Heterogeneity

In a recent study, multiple color spectral karyotyping and high resolution FISH technologies were applied to large numbers of cell lines and clinical samples in order to identify the pattern of genome alterations of various major cancer types. Interestingly, the common features that were observed in all of these major cancer types were increased levels of non-clonal chromosome aberrations (NCCAs), rather than the expected clonal chromosome aberrations. See, e.g., Heng, H. H., Bremer, S. W., et al. Cancer progression by non-clonal chromosome aberrations. J. Cell Biochem. 98:1424-1435 (2006).

Traditionally, cytogenetic studies have been solely focused on clonal chromosome aberrations, as some of these marker chromosomes have been linked to specific diseases as well as particular genes, such as the MCR/ABL fusion gene identified in CML patients. NCCAs, in contrast, have previously been thought of as background “noise” with no biological significance, as there seemed to be no clear pattern according to the concept of clonal expansion. To make sense of these findings, the system control principle may be effectively used to study cancer systems. Specifically, it is hypothesized that by using a system control approach, the system dynamics could be measured by determining the levels of seemingly random motion within the system. When the presence of NCCAs were selected as a method to measure genome system instability, increased frequencies of NCCAs were detected from genetically unstable cell lines, including inherently genetically unstable lines, stable lines induced to be unstable by various treatments, or stable lines with over-expressed cancer genes. Moreover, numerous factors that contribute to biological system instability have also been linked to increased NCCAs. Table 2 below enumerates a number of factors that can cause an increase in NCCA frequencies.

TABLE 2
Factors That Increase NCCA Frequencies

Genetic/epigenetic factors
Gene mutation
Epigenetic response
Genome variation
Physiological/pathological processes
Aging
Wound
Cell death
Inflammation
Environmental stress
Oncovirus
Vaccination
Carcinogen
Radiation
Experimental manipulations
Culture temperature
Nutrition status
Protein over expression
siRNA knock-out
Targeting protein degradation
Abnormal stroma-cell interaction
Drug resistance
Endoplasmic reticulum stress
Induced cell death
Transitions of immortalization/transformation/metastasis

When a biological system is unstable, increased dynamics can be detected at multiple levels of the genetic and epigenetic organization. For example, increased NCCAs were observed in cell lines with increased open chromatin structure. See, e.g., Dunn, K. L., He, S., et al. Increased genomic instability and altered chromosomal protein phosphorylation timing in HRAS transformed mouse fibroblasts. Genes Chromosomes Cancer 48:397-409 (2009). Therefore, by comparing various causes and the potential biological functions of NCCAs, it is clear that NCCAs reflect increased system dynamics and indeed characterize genome system heterogeneity.

### The Level of NCCAs Reflects Both Internally- and Externally-Induced Instability

Using an in vitro immortalization model, there are two phases of karyotypic evolution that have been observed called the “punctuated” and “stepwise” phases. See, e.g., Heng, H. H., Bremer, S. W., et al. Cancer progression by non-clonal chromosome aberrations. J. Cell Biochem. 98:1424-1435 (2006). Within the punctuated or discontinuous phase, non-clonal chromosome aberrations (NCCAs) dominate along with many short-lived transitional clonal chromosomal aberrations (CCAs); whereas in the stepwise phase, a given CCA dominates with low levels of NCCAs. Since the increased level of NCCAs reflects a system's instability, it is clear that such population instability can be generated either by internal changes (e.g., shortening of telomeres, loss of system constraints, and the like) or by environmentally-induced stress (e.g., chemotherapy-treatment, culture conditions, and the like). Accordingly, the frequencies of NCCAs can be used as an index to measure instability or population diversity and can be a determinant of the system's stage. Thus, even though instability can lead to heterogeneity, and heterogeneity can reflect levels of instability, both of them can be measured by the level of NCCAs. Interestingly, it is postulated that heterogeneity might be able to further generate instability, as heterogeneity itself might function as a stress applied to a system. In general terms, system instability and heterogeneity are, at a minimum, very-closley linked and may even refer to essentially to the same thing. The pattern of NCCAs also illustrates the difference between normal tissue and cancer tissue. In non-cancerous tissue, there is a balance between stability and heterogeneity such that the frequency of NCCAs is very low. In contrast, for cancer progression and drug resistance to occur, it is believed that a less stable status has to form, coupled with increased heterogeneity.

### Genome-Based Heterogeneity Promotes Tumorigenicity

In order to demonstrate that genome-based heterogeneity is a main contributing factor in cancer evolution, it is necessary to link heterogeneity to tumorigenicity. A recent study which compared five well-characterized cancer models that have been linked to specific molecular mechanisms, failed to identify any common genetic pattern or pathway among such mechanisms. The only pattern common to all the cancer models was increased levels of non-clonal chromosome aberrations (NCCAs) or karyotypic heterogeneity. Cancer formation appears to be a simplified evolutionary event based upon population heterogeneity and probability. Any unique NCCA represents one probability, as they each represent a unique genome system coupled to different pathways. See, e.g., Heng, H. H., Bremer, S. W., et al. Cancer progression by non-clonal chromosome aberrations. J. Cell Biochem. 98:1424-1435 (2006). According to the genome-centric concept (see, e.g., Heng, H. H. The genome-centric concept: re-synthesis of evolutionary theory. BioEssays 31:512-525 (2009)), in order to monitor genome system replacement or macro-somatic evolution, the study of specific genes or even epigenetic regulation will most likely be less useful than analysis at the genome level, in terms of understanding overall cancer evolution and the “control” of the associated biological system. Moreover, somatic evolution focused at the genome level also explains why there are high levels of stochastic elements both, at the gene and epigenetic levels.

Interestingly, it turns out that lower levels of “randomness” are essential for higher levels of regulation when facing a drastically changed environment. In a sense, the genome context allows environments to select certain forms of stochastic change from the potential responses. By “giving up” detailed control at the lower level, the system can have a less conflicted and more effective control at the higher level. The finding that genome-based heterogeneity can effectively predict tumorigenicity reconciles numerous conflicting theories regarding the mechanisms of cancer.

In brief, the pre-conditions for the occurrence of cancer evolution are initiated by an unbalanced relationship between system heterogeneity and homeostasis; wherein system homeostasis can be considered an opposite force to system heterogeneity. Similar to the sexual reproduction filter that constrains the genome level's alterations to prevent macro-evolution, the multiple levels of homeostasis is the system constraint that prevents somatic macro-evolution. In a human-centric ideal scenario, within the multiple levels of homeostasis, environmental stress should be counter-acted by epigenetic regulation wherein: (i) disturbances of metabolic status should be recovered; (ii) the errors of DNA replication should be repaired; (iii) altered cells should be eliminated by apoptotic mechanisms; (iv) abnormal clones should be constrained by the tissue architecture; and (v) cancer cells should be eliminated by the immune system. In contrast, in a cancer-defined ideal scenario, the break down of homeostasis is the key to the cancer's success. Unfortunately, continually evolving systems are the way of life and cannot be totally prevented. In a sense, cancer is the price, e.g., humans pay for evolution, as an interaction between system heterogeneity and homeostasis (like the relationship between NCCAs and CCAs) will not be ended so long as the system continues to evolve.

From these findings, it seems logical to argue that the multiple levels of homeostasis are more important than genetic factors in constraining cancer, as alterations of system homeostasis, rather than individual genetic alterations, are responsible for the majority of cancers. Accordingly, in this genomic heterogeneity-based paradigm: (i) the robustness of a network, the reversible features of epigenetic regulation; (ii) tissue architecture; and (iii) the immune-system will play a more important role than individual genetic alterations. See, e.g., Martien, S., Abbadie, C. Acquisition of oxidative DNA damage during senescence: the first step toward carcinogenesis? Ann. N.Y. Acad. Sci. 1119:51-63 (2007). The aforementioned non-genetic features are defined by genome context, as different species display different networks and different potential responses towards stress, as well as different profiles of epigenetic patterns (i.e., most of the epigenetic landscape is determined by the genome). See, e.g., Heng, H. H. The genome-centric concept: re-synthesis of evolutionary theory. BioEssays 31:512-525 (2009). More importantly, when examining biological evolution (e.g., cancer progression), one must remember that inheritance is a key player in the evolutionary process; as without inheritance there would be no Darwinian evolution. Lastly, many levels of homeostasis/heterogeneity are clearly linked to the genetic stability/instability of the system. Interestingly, as each layer of homeostasis is broken down by cancer cells, the genome contexts are different from the constrained cell populations. See, e.g., Id. Even with drug resistance, the newly emerged cellular survivors display altered karyotypes. In this case, new systems are formed from karyotypic heterogeneity that breaks down the constraints of drug treatment.

### The Relationship Among Epigenetic Change, Gene Mutation, and Genome Alteration

Karyotypic evolution (system replacement) occurs during macro-evolution, while gene mutations are mainly linked to micro-evolution. See, e.g., Heng, H. H. The genome-centric concept: re-synthesis of evolutionary theory. BioEssays 31:512-525 (2009). Due to the involvement of global gene regulation (i.e., chromatin remodeling and genetic network regulation) and reversible features, epigenetic alteration can be considered as the prototype of genetic alterations and in particular genome level alterations. Therefore, it is likely that epigenetic alteration is an initial response when the genome system is under stress (see, e.g., Feinberg, A. P., Ohlsson, R., Henikoff, S. The epigenetic progenitor origin of human cancer. Nat. Rev. Genet. 7:21-32 (2006)), which provides an increased probability for evolution dynamics to occur within a given genome context. When changes are selected by the evolutionary process, these changes can be fixed either at a specific gene level or at the genome level (achieving the transition from epigenetic to genetic changes). Interestingly, a new run of epigenetic alteration can occur from newly changed genome topology (i.e., the genome defines the epigenetic potential). Therefore, the same epigenetic changes might have different biological meaning when occurring within different genome systems. For example, the hypomethylation of DNA can have unpredictable effects in terms of promoting or inhibiting cancer formation. See, e.g., Esteller, M. Epigenetics in cancer. New Engl. J. Med. 358:1148-1159 (2008).

As for the gene-genome relationship, any genome alteration will generate high levels of gene expression changes. Some gene mutations that involve genome integrity can contribute to genome alteration, but only the genome level changes define a new system. In other words, karyotypic changes are the point of no return for systems and both gene mutation and epigenetic alteration can contribute to this process. Based on new findings that there is a collaborative relationship between genes and regulators/organizers, such as that between DNA sequences and chromosomal location, gene expression status, and inserted genes, it follows that the understanding of the overall contribution of epigenetic regulation should not focus solely on tumor suppressor genes, but rather focus on system dynamics and evolve-ability. The genome context also defines the pattern of epigenetic regulation. This is exemplified by the fact that the epigenetic features are species-specific phenomena and macro-evolution acts on the genome package level with a certain stochastic feature ensured by epigenetic regulation. One advantage of epigenetic regulation is the alteration of system dynamics without too much specificity that can be effectively adapted by the combination of genome context and environmental stress. While it is true that inappropriate gene silencing occurs involving tumor suppressor genes, the more profound changes are the increased overall level of systems dynamics, which contributes to epigenetic heterogeneity. This is illustrated by the fact that the DNA of cancer cells is generally hypomethylated leading to higher levels of gene expression for massive numbers of genes. It is possible that at certain stages of cancer progression, some pathways become dominant, but this process is stochastic and unpredictable as there are so many pathways that could be dominant depending on the possible combinations of genome context and environment.

Macro-evolutionary selection mainly functions at the genome level (different genome systems are defined by different karyotypes coupled to unique gene expression profiles). See, e.g., Heng, H. H. The genome-centric concept: re-synthesis of evolutionary theory. BioEssays 31:512-525 (2009). Micro-evolution mainly involves gene mutation and epigenetic responses that are responsible for a given system's micro-evolution or adaptation. In eukaryotic evolution, due to a fixed genome framework preserved by the sexual reproduction filter following speciation, an increased system complexity relies more on a layer of epigenetic regulation and copy number variation. This is important for micro-evolution and adaptation as environments are constantly changing while the framework of the genome is mainly stable. In contrast, during somatic evolution, due to the lack of the aforementioned “sexual filters” to constrain the genome, genome level replacement becomes dominant thus epigenetic regulation becomes less important for the genome to adapt to a changing environment. In this case, many gene mutations and epigenetic responses or even non-genetic stresses can trigger genomic macro-evolution, making it difficult to identify common patterns of specific gene mutation or epigenetic responses that are responsible for cancer evolution. The involvement of gene mutations/copy number variation and epigenetic regulation becomes more dynamic and less orderly as well.

### Measuring Heterogeneity at the Gene Level

Most evolutionary studies have focused on specific genetic loci using mixed cell populations, however, there are successful examples of using genetic heterogeneity of some key genetic loci or events (e.g., clonal diversity of p53 and ploidy abnormalities) to predict the clinical outcomes of, for example, esophageal adenocarcinoma. See, e.g., Maley, C. C., Galipeau, P. C., et al. Genetic clonal diversity predicts progression to esophageal adenocarcinoma. Nat. Genet. 38:468-473 (2006). When closely examining the contribution of various genetic factors, it is clear that many of the genetic loci or events are only significantly linked to tumorigenicity when they contribute to system instability (which is closely linked to genome level heterogeneity). The tracing of genetic clonal diversity using individual genes essentially results in the monitoring of the secondary effects of system heterogeneity. According to the genome-centric concept, focusing on gene level heterogeneity is not as effective as monitoring genome level heterogeneity, as in order to contribute to cancer evolution, these gene level changes are significant enough to impact genome level heterogeneity.

Most genes do not significantly contribute to system instability, and thus are not a useful measure of genetic heterogeneity at the genome level. In addition, the effects of gene level heterogeneity are also limited by genome level heterogeneity constraints. Finally, current methods used to trace genetic loci heterogeneity are not accurate, as the admixture of DNA from different cells will wash away the true high level of heterogeneity and only display the heterogeneity of dominant clonal populations. It is thus problematic using individual genetic loci data to model macro-evolutionary processes.

### Studying Epigenetic Heterogeneity

Epigenetic heterogeneity has long been observed and studied. Different methylation patterns are present between and within individuals, as exemplified in, e.g., intestinal crypts and endometrial glands. See, e.g., Shibata, D., Tavare, S. Counting divisions in a human somatic cell tree: how, what and why? Cell Cycle 5:610-614 (2006). Despite average methylation increases with age, high heterogeneity is evident. It is a challenge to link age-related methylation to a specific function. The fact that epigenetic alterations may occur at different stages of tumorigenesis further complicates the issue of how to analyze epigenetic heterogeneity. See, e.g., Kurdistani, S. K. Histone modifications as markers of cancer prognosis: a cellular view. Br. J. Cancer 97:1-5 (2007). For example, it is known that epigenetic patterns are altered in pre-cancer tissues, and high levels of cellular epigenetic heterogeneity can be visualized in cancer tissue by monitoring the histone H3 lysine 18 acetylation. In fact, epigenetic heterogeneity is an important feature of the epigenetic theory of cancer initiation. According to this concept, chronic insults repeatedly injure and transiently excite many cells in particular tissues and these excited cells undergo epigenetic response, and initiate tumorigenesis (through epigenetic heterogeneity). See, e.g., Jaffe, L. F. A calcium-based theory of carcinogenesis. Adv. Cancer Res. 94:231-263 (2005). Despite the awareness of epigenetic heterogeneity, few examples have been presented to measure epigenetic heterogeneity or use that data to predict the probability of tumorigenicity.

Monitoring non-clonal chromosome aberrations (NCCAs) defined genome level heterogeneity is an effective way to study overall heterogeneity. Using karyotypic heterogeneity to measure genome level heterogeneity is a new approach to link population diversity to tumorigenicity. See, e.g., Ye, C. J., Stevens, J. B., et al. Genome based cell population heterogeneity promotes tumorigenicity: The evolutionary mechanism of cancer. J. Cell Physiol. 219:288-300 (2009). Compared with measuring heterogeneity both at the gene and epigenetic level, genome level measurements should have greater predictive power. This approach fits well with the genome-centric concept of cancer evolution as genes are genetic material for the system while epigenetic regulation is one layer of control responsible for genome modification, such as regulation of certain tissue specific genes. These two levels belong to the lower level components of a given genome. Studies linking genome level heterogeneity to tumorigenicity have led to a formula that illustrates the relationship between the evolutionary mechanism of cancer and all possible molecular mechanisms:

Evolutionary Mechanisms=Σ Individual Molecular Mechanisms

Accordingly, the most effective way to monitor cancer progression is to use the evolutionary mechanism approach. The evolutionary mechanism of cancer can be explained by three main components: instability imparts heterogeneity, which is acted on by natural selection. As each component can be impacted by a great number of genetic/epigenetic and environmental factors, it would be extremely challenging to trace each of these unlimited molecular mechanisms, where each NCCA defines a system with specific pathways (and NCCAs represent the heterogeneity of cancer evolution). On the other hand, it is relatively easy to monitor patterns of evolution, by measuring population diversity, and examining the dynamic relationship between NCCAs and clonal chromosomal aberrations (CCAs).

Since cancer evolution is driven by macro-evolution and the genome is the platform of macro-selection, all genetic and epigenetic alterations will not have the same impact but can contribute to the adaptation of the genome package. See, e.g., Heng, H. H. The genome-centric concept: re-synthesis of evolutionary theory. BioEssays 31:512-525 (2009). Therefore, gene or epigenetic heterogeneity may or may not be significant enough to impact genome level evolutionary outcomes. The implication of this finding is significant. It explains why there are so many genetic/epigenetic and environmental factors linked to cancer based on small numbers of individual patients, yet, most of them are not shared among large patient populations. Two conclusions may be derived from this analysis:
(i) It is incorrect to validate mutations using large patient populations if these mutations have low penetration within a population. If the “average sample” approach is used to compare large samples to identify common patterns, the majority of lower penetrant mutations will be washed away by the analysis, becoming statistically insignificant for a population despite their strong association to individual tumors. Clearly, it is a challenge to develop an effective means to evaluate the contributions of individual mutations in a highly heterogeneous background; and
(ii) The predictability of cancer can be accomplished by measuring the system heterogeneity that is shared by most patients, rather than characterize each of the individual factors that contributes to cancer.

In conclusion, the multiple levels of genetic and epigenetic alteration are the key elements of cancer evolution. For normal cells to function under a constantly changing environment including the cellular environment and the overall individual health status, the key is to maintain system homeostasis through system dynamics without significantly increasing heterogeneity especially at the genome level. It must be remembered that dynamics are necessary, otherwise biological processes will not function in a changing environment. However, if there is too great a dynamic interaction, the drastically increased system heterogeneity will trigger cancer evolution. Unfortunately, many factors, including but not limited to: the genetic background, the aging process, stochastic genetic and epigenetic changes, and environmental stress, will unavoidably alter the balance between heterogeneity and homeostasis favoring cancer evolution. Understanding the genome-centric concept of cancer evolution will help to develop applications, treatments, and an experimental system to monitor and measure system dynamics without making the system impossibly difficult to access by requiring monitoring of the lower levels of genetic and epigenetic alteration. Thus, the genome-centric concept will serve as a guide when applying genome level heterogeneity to the clinical challenges of cancer, as well as other common non-neoplastic diseases.

### III. Heterogeneous Seminal Biological Capabilities of Cancer

A recent review article characterizes eight (8) seminal biological capabilities which are acquired during the multistep development of human cancer and tumors. See, Hanahan, D., Weinberg, R. A. Hallmarks of cancer: the next generation. Cell 144:646-674, (2011). These biological capabilities constitute an organizing paradigm for understanding the inherent complexities of neoplastic disease and include: (i) resisting cell death (apoptosis); (ii) enabling replicative immortality; (iii) sustaining proliferative signaling; (iv) evading growth suppressors; (v) inducing angiogenesis; (vi) activating invasion and metastasis; (vii) reprogramming of energy metabolism; and (viii) evading immune destruction. Working in concert with these biological capabilities are genomic instability (which generates the genetic diversity that expedites their acquisition) and inflammation (which fosters multiple hallmark functions). In addition to the cancer cells themselves, tumors also exhibit another dimension of biological complexity in that they contain a repertoire of recruited, ostensibly normal cells that contribute to the acquisition of the aforementioned biological capabilities by creating a “tumor microenvironment”.

Implicit in this organizing paradigm for understanding the inherent complexities of neoplastic disease is the notion that as normal cells progressively evolve to a neoplastic state, they acquire a succession of these seminal biological capabilities, and that the multistep process of human tumor pathogenesis could be rationalized by the need of incipient cancer cells to acquire the traits that enable them to become tumorigenic and ultimately malignant. A long-held, but erroneous, proposition held that tumors are insular masses of proliferating cancer cells. In truth, tumors are complex tissues composed of multiple distinct cell types that participate in heterotypic interactions with one another. For example, there is the recruitment of normal cells, which form tumor-associated stroma, to act as active participants in tumorigenesis rather than passive bystanders; as such, these stromal cells contribute to the development and expression of certain seminal biological capabilities. It is now understood that the biology of tumors can no longer be understood simply by enumerating the traits of the cancer cells, but instead must encompass the numerous contributions of the entire “tumor microenvironment” to process of tumorigenesis.

The eight seminal biological capabilities of cancer are distinctive and complementary characteristics that enable tumor growth and metastatic dissemination. Each of these eight seminal biological capabilities assist in providing a solid foundation for the understanding the biology of cancer and will be discussed individually below.

(i) Resisting Programmed Cell Death (Apoptosis)

The concept that programmed cell death by apoptosis serves as a natural impediment to cancer development has been established by numerous functional studies. See, e.g., Adams, J. M., Cory, S. The Bcl-2 apoptotic switch in cancer development and therapy. Oncogene 26:1324-1337 (2007). Elucidation of the signaling circuitry governing the apoptotic program has revealed how apoptosis is triggered in response to various physiologic stresses that cancer cells experience during the course of tumorigenesis or as a result of anti-cancer therapy. Notable among the apoptosis-inducing stresses are signaling imbalances resulting from elevated levels of oncogene signaling and DNA damage associated with hyperproliferation. Yet other research has revealed how apoptosis is attenuated in those tumors that succeed in progressing to states of high-grade malignancy and resistance to therapy.

The apoptotic machinery is composed of both upstream regulators and downstream effector components. See, e.g., Id. The regulators, in turn, are divided into two major circuits: (i) one receiving and processing extracellular death-inducing signals (the extrinsic apoptotic program) involving, e.g., the Fas ligand/Fas receptor; and (ii) one sensing and integrating a variety of signals of intracellular origin (the intrinsic program). Each culminates in activation of a normally latent protease, which proceeds to initiate a cascade of proteolysis involving effector caspases responsible for the execution phase of apoptosis, in which the cell is progressively disassembled and then consumed, both by its neighbors and/or by phagocytic cells. It should be noted that the intrinsic apoptotic program is more widely implicated as a barrier to cancer pathogenesis.

The “apoptotic trigger” that conveys signals between the regulators and effectors is controlled by the counter-balancing of pro- and anti-apoptotic members of the Bcl-2 family of regulatory proteins. See, e.g., Adams, J. M., Cory, S. The Bcl-2 apoptotic switch in cancer development and therapy. Oncogene 26:1324-1337 (2007). The archetype, Bcl-2, along with its closest relatives (i.e., Bcl-xL, Bcl-w, Mcl-1, A1) are inhibitors of apoptosis, which act in a large part by binding to and thereby suppressing two pro-apoptotic triggering proteins (Bax and Bak) embedded in the mitochondrial outer membrane. When relieved of inhibition by their anti-apoptotic relatives, Bax and Bak disrupt the integrity of the outer mitochondrial membrane causing the release of pro-apoptotic signaling proteins—the most important of which is cytochrome c. The released cytochrome c activates, in turn, a cascade of caspases that act via their proteolytic activities to induce the multiple cellular changes associated with the apoptotic program. Bax and Bak share protein-protein interaction domains (i.e., BH3 motifs) with the antiapoptotic Bcl-2-like proteins that mediate their various physical interactions.

Although the cellular conditions that trigger apoptosis remain to be fully elucidated, several cellular abnormality sensors that play key roles in tumor development have been identified. Most notable is a DNA damage sensor that functions via the TP53 tumor suppressor. See, e.g., Junttila, M. R., Evan, G. I. p53—a jack of all trades but master of none. Nat. Rev. Cancer 9:821-829 (2009). TP53 induces apoptosis by up-regulating expression of the Noxa and Puma BH3-only proteins in response to substantial levels of DNA breaks and other chromosomal abnormalities. Another condition leading to cell death involves hyperactive signaling by certain oncoproteins (e.g., Myc) which triggers apoptosis unless counter-balanced by anti-apoptotic factors. See, e.g., Id.

Tumor cells evolve a variety of strategies to limit or circumvent apoptosis. Most common is the loss of TP53 tumor suppressor function, which eliminates this critical damage sensor from the apoptosis-inducing circuitry. Alternatively, tumors may also achieve similar results by: (i) increasing expression of anti-apoptotic regulators (e.g., Bcl-2); (ii) increasing survival signals by down-regulating pro-apoptotic factors (e.g., Bax); or (iii) short-circuiting the extrinsic ligand-induced death pathway. The multiplicity of apoptosis-avoiding mechanisms presumably reflects the diversity of apoptosis-inducing signals that cancer cell populations encounter during their evolution to the malignant state.

### Mediation of Both Tumor Cell Survival and Death by Autophagy

Autophagy (a catabolic process involving the degradation of a cell's own intracellular components through the lysosomal machinery). represents an important cellular physiological response that, like apoptosis, normally operates at low, basal levels. However, in certain states of cellular stress (e.g., nutrient deficiency) it can be strongly induced. See, e.g., Levine, B., Kroemer, G. Autophagy in the pathogenesis of disease. Cell 132:27-42 (2008). In autophagy, intracellular vesicles termed autophagosomes envelope intracellular organelles and then fuse with lysosomes wherein the degradation occurs. In this manner, low molecular weight metabolites are generated that support survival in the stressed, nutrient-limited environments experienced by many cancer cells. Autophagy enables cells to break down cellular organelles (e.g., ribosomes and mitochondria), thus allowing the resulting catabolites to be recycled and thus used for biosynthesis and energy metabolism.

Like apoptosis, the autophagy machinery has both regulatory and effector components. Among the latter are proteins that mediate autophagosome formation and delivery to lysosomes. Recent research has revealed intersections between the regulatory circuits governing autophagy, apoptosis, and cellular homeostasis. By way of example, the signaling pathway involving the PI3-kinase, AKT, and mTOR kinases (which is stimulated by survival signals to block apoptosis) similarly inhibits autophagy. Conversly, when survival signals are insufficient, the PI3K signaling pathway is downregulated, with the result that autophagy and/or apoptosis may be induced. See, e.g., Sinha, S., Levine, B. The autophagy effector Beclin 1: a novel BH3-only protein. Oncogene 27 (Suppl. 1):S137-S148 (2008).

Paradoxically, nutrient starvation, radiotherapy, and certain cytotoxic drugs can induce elevated levels of autophagy that are apparently cytoprotective for cancer cells, impairing rather than accentuating the killing actions of these stress-inducing situations. Moreover, severely stressed cancer cells have been shown to shrink via autophagy to a state of reversible dormancy. This survival response may enable the persistence and eventual regrowth of some late stage tumors following treatment with potent anticancer agents. Thus, in analogy to TGF-β signaling, which can be tumor suppressing at early stages of tumorigenesis and tumor promoting later on, autophagy seems to have conflicting effects on tumor cells and thus tumor progression. See, e.g., White, E., DiPaola, R. S. The double-edged sword of autophagy modulation in cancer. Clin. Cancer Res. 15:5308-5316 (2009). These results suggest that induction of autophagy can serve as a barrier to tumorigenesis that may operate independently of or in concert with apoptosis. Accordingly, autophagy appears to represent yet another barrier that needs to be circumvented during tumor development.

### The Proinflammatory and Tumor-Promoting Potential of Necrosis

In contrast to apoptosis, necrotic cells become bloated and explode, releasing their contents into the local tissue microenvironment. Research has clearly established that cell death by necrosis is clearly under genetic control in some circumstances, rather than merely being a random and undirected process. See, e.g., Galluzzi, L., Kroemer, G. Necroptosis: a specialized pathway of programmed necrosis. Cell 135:1161-1163 (2008).

Perhaps more importantly, necrotic cell death releases pro-inflammatory signals into the surrounding tissue microenvironment, in contrast to apoptosis and autophagy. As a consequence, necrotic cells can recruit inflammatory cells of the immune system, whose dedicated function is to survey the extent of tissue damage and remove associated necrotic debris. See, e.g., Grivennikov, S. I., Greten, F. R., Karin, M. Immunity, inflammation, and cancer. Cell 140:883-899 (2010). Interestingly, recent lines of evidence indicate that immune inflammatory cells can be actively tumor-promoting, given that such cells have been shown to be capable of fostering angiogenesis, cancer cell proliferation, and invasiveness. Furthermore, necrotic cells can release bioactive regulatory factors (e.g., IL-1a), which can directly stimulate neighboring viable cells to proliferate, with the potential to facilitate neoplastic progression. See, e.g., Id. In accord, neoplasias and potentially invasive and/or metastatic tumors may gain an advantage by tolerating some degree of necrotic cell death, and in doing so recruit tumor-promoting inflammatory cells that bring growth-stimulating factors to the surviving cells within these growths.

(ii) Enabling Cellular Replicative Immortality

It is widely accepted that cancer cells require unlimited replicative potential in order to generate macroscopic tumors. This capability stands in marked contrast to the behavior of the cells in most normal cell lineages in the body, which are able to only undergo a limited number of successive cell growth-and-division cycles. This limitation has been associated with two distinct barriers to proliferation: (i) senescence (a typically irreversible entrance into a nonproliferative but viable state) and (ii) crisis (which involves cell death). Accordingly, when cells are propagated in vitro, repeated cycles of cell division lead first to induction of senescence and then, for those cells that succeed in circumventing this barrier, to a crisis phase, in which the great majority of cells in the population die. However, on rare occasion, cells emerge from a population in crisis and exhibit unlimited replicative potential. This transition has been termed immortalization. It should be noted that most established cell lines possess this characteristic by virtue of their ability to proliferate in culture without evidence of either senescence or crisis.

Experimental evidence indicates that telomeres protecting the ends of chromosomes are centrally involved in the capability for unlimited proliferation. The telomeres, composed of multiple tandem hexanucleotide repeats, shorten progressively in non-immortalized cells propagated in culture. This telomeric shortening eventually causes them to lose the ability to protect the ends of chromosomal DNAs from end-to-end fusions; wherein such fusions generate unstable dicentric chromosomes whose resolution results in a scrambling of karyotype that threatens cell viability. Accordingly, the length of telomeric DNA in a cell dictates how many successive cell generations its progeny can pass through before telomeres are largely eroded and have consequently lost their protective functions, triggering entrance into crisis. See, e.g., Blasco, M. A. Telomeres and human disease: aging, cancer and beyond. Nat. Rev. Genet. 6: 611-622 (2005).

Telomerase, the specialized DNA polymerase that adds telomere repeat segments to the ends of telomeric DNA, is almost absent in non-immortalized cells. However, it is expressed at functionally significant levels in the vast majority (˜90%) of spontaneously immortalized cells, including human cancer cells. Therefore, by extending telomeric DNA, telomerase is able to counter the progressive telomere erosion that would otherwise occur in its absence. The presence of telomerase activity, either in spontaneously immortalized cells or in cells engineered to express the enzyme, is correlated with a resistance to induction of both senescence and crisis/apoptosis. Conversely, suppression of telomerase activity leads to telomere shortening and to activation of one of these proliferative barriers. The two barriers to proliferation (i.e., senescence and crisis/apoptosis) have been rationalized as crucial anti-cancer defenses that are hard-wired into our cells, being deployed to impede the outgrowth of clones of pre-neoplastic and frankly neoplastic cells. According to this rational, most incipient neoplasias exhaust their given number of replicative doublings and are stopped by one the aforementioned barriers. The eventual immortalization of rare variant cells that proceed to form tumors has been attributed to their ability to maintain telomeric DNA at lengths sufficient to avoid triggering senescence or apoptosis, achieved most commonly by up-regulating expression of telomerase or, less frequently, via an alternative recombination-based telomere maintenance mechanism. In accord, telomeric shortening has come to be viewed as a “clocking device” that determines the limited replicative potential of normal cells and is thus, a barrier that must be overcome by cancer cells.

### Replicative Senescence

Whereas telomere maintenance has been increasingly substantiated as a condition critical to the neoplastic state, the concept of replication-induced senescence as a general barrier requires refinement and reformulation. Differences in telomere structure and function in mouse versus human cells have also complicated investigation of the roles of telomeres and telomerase in replicative senescence. Recent experiments have revealed that the induction of senescence in certain cultured cells can be delayed and possibly even eliminated by the use of improved cell culture conditions, suggesting that recently explanted primary cells may be able to proliferate unimpeded in culture up the point of crisis and the associated induction of apoptosis triggered by critically shortened telomeres. See, e.g., Ince, T. A., Richardson, A. L., et al., Transformation of different human breast epithelial cell types leads to distinct tumor phenotypes. Cancer Cell 12:160-170 (2007).

In contrast, experiments in mice which were genetically engineered to lack telomerase indicate that the consequently shortened telomeres can shunt pre-malignant cells into a senescent state that contributes (along with apoptosis) to attenuated tumorigenesis in mice genetically predetermined to develop specific forms of cancer. See, e.g., Artandi, S. E., DePinho, R. A. Telomeres and telomerase in cancer. Carcinogenesis 31:9-18 (2010). Such telomerase null mice with highly eroded telomeres exhibit multi-organ dysfunction and abnormalities that include evidence for both senescence and apoptosis, which is perhaps analogous to the senescence and apoptosis observed in cell culture. In summation, cell senescence is emerging as a protective barrier to neoplastic expansion that can be triggered by various proliferation-associated abnormalities (e.g., high levels of oncogenic signaling and sub-critical shortening of telomeres).

### Delayed Activation of Telomerase May Both Limit and Foster Neoplastic Progression

There is now evidence that clones of incipient cancer cells often experience telomere loss-induced crisis relatively early during the course of multistep tumor progression due to their inability to express significant levels of telomerase. Thus, extensively eroded telomeres have been documented in pre-malignant growths through the use of fluorescence in situ hybridization (FISH), which has also revealed the end-to-end chromosomal fusions that signal telomere failure and crisis. See, e.g., Kawai, T., Hiroi, S., Nakanishi, K., Meeker, A. K. (2007). Telomere length and telomerase expression in atypical adenomatous hyperplasia and small bronchioloalveolar carcinoma of the lung. Am. J. Clin. Pathol. 127:254-262 (2007). These results also suggest that such cells have passed through a substantial number of successive telomere-shortening cell divisions during their evolution from fully normal cells-of-origin. Accordingly, the development of some human neoplasias may be aborted by telomere-induced crisis long before they succeed in becoming macroscopic, frankly neoplastic growths.

In contrast, the lack of TP53-mediated “surveillance” of genomic integrity may permit other incipient neoplasias to survive initial telomere erosion and attendant chromosomal breakage-fusion-bridge (BFB) cycles. The genomic alterations resulting from these BFB cycles (including deletions and amplifications of chromosomal segments) apparently serve to increase the mutability of the genome, thereby accelerating the acquisition of mutant oncogenes and tumor suppressor genes. The realization that impaired telomere function can actually foster tumor progression has come from the study of mutant mice that lack both p53 and telomerase function. See, e.g., Artandi, S. E., DePinho, R. A. Telomeres and telomerase in cancer. Carcinogenesis 31:9-18 (2010). However, it should be noted that the hypothysis that these two aforementioned defects can cooperatively enhance human tumorigenesis has not yet been quantitatively ascertained.

### Recently Ascertained Functions of Telomerase

As discussed previously, telomerase has the ability to elongate and maintain telomeric DNA. However, more recently, it has been demonstrated that telomerase also possesses novel functions that are relevant to cell proliferation, but unrelated to telomeric DNA maintenance. These novel functions, and in particular the function of its protein subunit TERT, have been demonstrated in conditions where the telomerase enzymatic activity has been eliminated. By way of example, telomere-independent functions of TERT/telomerase include: (i) the ability of TERT to amplify signaling by the Wnt pathway by serving as a cofactor of the β-catenin/LEF transcription factor complex; (ii) enhancement of cell proliferation and/or resistance to apoptosis; (iii) involvement in DNA-damage repair; and (iv) RNA-dependent RNA polymerase function. See, e.g., Cong, Y., Shay, J. W. Actions of human telomerase beyond telomeres. Cell Res. 18:725-732 (2008).

(iii) Sustaining Proliferative Signaling

One of the most fundamental traits of cancer cells involves their ability to sustain chronic proliferation. Normal tissues carefully control the production and release of growth-promoting signals that instruct entry into and progression through the cell growth and division cycle, thereby ensuring a homeostasis of cell number and thus maintenance of normal tissue architecture and function. Cancer cells, by deregulating these signals, obtain control of their ultimate destiny. The enabling signals are conveyed, in large part, by various growth factors that bind to cell-surface receptors, which typically contain intracellular tyrosine kinase domains. The latter subsequently proceed to emit signals via branched intracellular signaling pathways that regulate progression through the cell cycle, as well as cell growth. These signals also influence numerous other cell-biological properties (e.g., cell survival, energy metabolism, and the like).

Unfortunately, both the precise identities and sources of the proliferative signals operating within normal tissues remain poorly elucidated. Moreover, relatively little is understood regarding the mechanisms controlling the release of these mitogenic signals. The understanding of these mechanisms is complicated, in part, by the fact that the growth factor signals controlling cell number and position within tissues are thought to be transmitted in a temporally- and spatially-regulated manner from one cell to its neighbors. Such paracrine signaling is difficult to access experimentally. In addition, the bioavailability of growth factors is regulated by sequestration in the pericellular space and extracellular matrix, and by the actions of a complex network of proteases, sulfatases, and possibly other enzymes that liberate and activate them, apparently in a highly specific and localized fashion.

In contrast to normal cells, the mitogenic signaling in cancer cells is better understood. See, e.g., Lemmon, M. A., Schlessinger, J. Cell signaling by receptor tyrosine kinases. Cell 141:1117-1134 (2010). Cancer cells can acquire the capability to sustain proliferative signaling in a number of alternative ways. They may produce growth factor ligands themselves, to which they can respond via the expression of cognate receptors, resulting in autocrine proliferative stimulation. Alternatively, cancer cells may send signals to stimulate normal cells within the supporting tumor-associated stroma, which reciprocate by supplying the cancer cells with various growth factors. See, e.g., Cheng, N., Chytil, A., Shyr, Y., Joly, A., Moses, H. L. Transforming growth factor-beta signaling-deficient fibroblasts enhance hepatocyte growth factor signaling in mammary carcinoma cells to promote scattering and invasion. Mol. Cancer Res. 6:1521-1533 (2008). Receptor signaling can also be deregulated by elevating the levels of receptor proteins displayed at the cancer cell surface, rendering such cells hyperresponsive to otherwise-limiting amounts of growth factor ligand; the same outcome can result from structural alterations in the receptor molecules that facilitate ligand-independent firing. Growth factor independence may also derive from the constitutive activation of components of signaling pathways operating downstream of these receptors, obviating the need to stimulate these pathways by ligand-mediated receptor activation. Given that a number of distinct downstream signaling pathways radiate from a ligand-stimulated receptor, the activation of one or another of these downstream pathways (e.g., the pathway responding to the signal transducer) may only recapitulate a subset of the regulatory instructions transmitted by an activated receptor.

### Activation of Downstream Pathways by Somatic Mutations

High-throughput DNA sequencing analyses of cancer cell genomes have revealed somatic mutations in certain human tumors that predict constitutive activation of signaling circuits usually triggered by activated growth factor receptors. Thus, we now know that 40% of human melanomas contain activating mutations affecting the structure of the B-Raf protein, resulting in constitutive signaling through the Raf to mitogenactivated protein (MAP)-kinase pathway. Similarly, mutations in the catalytic subunit of phosphoinositide 3-kinase (PI3-kinase) isoforms are being detected in an array of tumor types, which serve to hyperactivate the PI3-kinase signaling circuitry, including its key Akt/PKB signal transducer. The advantages to tumor cells of activating upstream (receptor) versus downstream (transducer) signaling remain obscure, as does the functional impact of cross-talk between the multiple pathways radiating from growth factor receptors. See, e.g., Davies, M. A., Samuels, Y. Analysis of the genome to personalize therapy for melanoma. Oncogene 29:5545-5555 (2010).

### Disruptions of Negative-Feedback Mechanisms that Attenuate Proliferative Signaling

Recent results have highlighted the importance of negative feedback loops that normally operate to dampen various types of signaling and thereby ensure homeostatic regulation of the flux of signals coursing through the intracellular circuitry. See, e.g., Wertz, I. E., Dixit, V. M. Regulation of death receptor signaling by the ubiquitin system. Cell Death Differ. 17:14-24 (2010). Defects in these feedback mechanisms are capable of enhancing proliferative signaling. The prototype of this type of regulation involves the Ras oncoprotein; the oncogenic effects of Ras do not result from a hyperactivation of its signaling powers; instead, the oncogenic mutations affecting ras genes compromise Ras GTPase activity, which operates as an intrinsic negative-feedback mechanism that normally ensures that active signal transmission is transitory.

Analogous negative-feedback mechanisms operate at multiple nodes within the proliferative signaling circuitry. A prominent example involves the PTEN phosphatase, which counteracts PI3-kinase by degrading its product, phosphatidylinositol (3,4,5) triphosphate (PIP3). Loss-of-function mutations in PTEN amplify PI3K signaling and promote tumorigenesis in a variety of experimental models of cancer; in human tumors, PTEN expression is often lost by promoter. See, e.g., Jiang, B. H., Liu, L. Z. PI3K/PTEN signaling in angiogenesis and tumorigenesis. Adv. Cancer Res. 102:19-65 (2009). It is likely that compromised negative-feedback loops in this and other signaling pathways will prove to be widespread among human cancer cells and serve as an important means by which these cells can achieve proliferative independence. Moreover, disruption of such self-attenuating signaling may contribute to the development of adaptive resistance toward drugs targeting mitogenic signaling.

### Triggering of Cell Senescence by Excessive Proliferative Signaling

Early studies of oncogene action encouraged the notion that ever-increasing expression of such genes and the signals manifested in their protein products would result in correspondingly increased cancer cell proliferation and thus tumor growth. More recent research has undermined this notion, in that excessively elevated signaling by oncoproteins such as RAS, MYC, and RAF can provoke counteracting responses from cells, specifically induction of cell senescence and/or apoptosis. See, e.g., Collado, M., Serrano, M. Senescence in tumours: evidence from mice and humans. Nat. Rev. Cancer 10:51-57 (2010). For example, cultured cells expressing high levels of the Ras oncoprotein may enter into the nonproliferative, yet viable state, called senescence; in contrast, cells expressing lower levels of this protein may avoid senescence and continue to proliferate. Cells with morphological features of senescence, including enlarged cytoplasm, the absence of proliferation markers, and expression of the senescence-induced β-galactosidase enzyme, are abundant in the tissues of mice engineered to overexpress certain oncogenes and are prevalent in some cases of human melanoma. These ostensibly paradoxical responses seem to reflect intrinsic cellular defense mechanisms designed to eliminate cells experiencing excessive levels of certain types of signaling. Accordingly, the relative intensity of oncogenic signaling in cancer cells may represent compromises between maximal mitogenic stimulation and avoidance of these antiproliferative defenses. Alternatively, some cancer cells may adapt to high levels of oncogenic signaling by disabling their senescence- or apoptosis-inducing circuitry.

(iv) Evading Growth Suppressors

In addition to the seminal biological capability of inducing and sustaining positively acting growth-stimulatory signals, cancer cells must also circumvent powerful programs that negatively regulate cell proliferation. Many of these programs depend on the actions of tumor suppressor genes. Dozens of tumor suppressors that operate in various ways to limit cell growth and proliferation have been discovered through their characteristic inactivation in one or another form of animal or human cancer; many of these genes have been validated as bona fide tumor suppressors through gain- or loss-of-function experiments in mice. The two prototypical tumor suppressors encode the RB (retinoblastoma-associated) and TP53 proteins; they operate as central control nodes within two key complementary cellular regulatory circuits that govern the decisions of cells to proliferate or, alternatively, activate senescence and apoptotic programs. The RB protein integrates signals from diverse extracellular and intracellular sources and, in response, decides whether or not a cell should proceed through its growth-and-division cycle. See, e.g., Burkhart, D. L., Sage, J. Cellular mechanisms of tumour suppression by the retinoblastoma gene. Nat. Rev. Cancer 8:671-682 (2008). Cancer cells with defects in RB pathway function are thus missing the services of a critical gatekeeper of cell-cycle progression whose absence permits persistent cell proliferation. Whereas RB transduces growth-inhibitory signals that originate largely outside of the cell, TP53 receives inputs from stress and abnormality sensors that function within the cell's intracellular operating systems. By way of example, if the degree of damage to the cell's genome is excessive or if, e.g., the levels of nucleotide pools, growth-promoting signals, glucose, or oxygenation are suboptimal, TP53 can halt further cell-cycle progression until these aforementioned condition(s) have been normalized. Alternatively, in cases where there is overwhelming or irreparable damage to such cellular sub-systems, TP53 can trigger programmed cell death (i.e., apoptosis). It should be noted however, that the effects of activated TP53 are complex and highly context dependent, and vary by both cell type and by the severity/persistence of conditions of cell stress and genomic damage.

### Evasion of Cell-to-Cell Contact Inhibition

Decades of research have demonstrated that the cell-to-cell contacts formed by dense populations of normal cells propagated in two-dimensional culture operate to suppress further cell proliferation, yielding confluent cell monolayers. Importantly, such “contact inhibition” is abolished in various types of cancer cells in culture, suggesting that contact inhibition is an in vitro surrogate of a mechanism that operates in vivo to ensure normal tissue homeostasis. This mechanism is abrogated during the course of tumorigenesis. Although the mechanistic basis for this mode of growth control has remained obscure, the mechanisms of contact inhibition are beginning to be elucidated. One mechanism involves the product of the NF2 gene, long implicated as a tumor suppressor. Merlin (the cytoplasmic NF2 gene product) has been shown to orchestrate contact inhibition via coupling cell-surface adhesion molecules (e.g., E-cadherin) to transmembrane receptor tyrosine kinases (e.g., the EGF receptor). By this process, Merlin strengthens the adhesivity of cadherin-mediated cell-to-cell attachments. Additionally, by sequestering growth factor receptors, Merlin limits their ability to efficiently emit mitogenic signals. See, e.g., Curto, M., Cole, B. K., et al., Contact-dependent inhibition of EGFR signaling by Nf2/Merlin. J. Cell Biol. 177:893-903 (2007).

A second mechanism of contact inhibition involves the LKB1 epithelial polarity protein, which organizes epithelial structure and helps maintain tissue integrity. LKB1 can, for example, overrule the mitogenic effects of the powerful Myc oncogene when the latter is upregulated in organized, quiescent epithelial structures. In contrast, when LKB1 expression is suppressed, epithelial integrity is destabilized, and epithelial cells become susceptible to Myc-induced transformation. See, e.g., Partanen, J. I., Nieminen, A. I., Klefstrom, J. 3-D view to tumor suppression: Lkb1, polarity and the arrest of oncogenic c-Myc. Cell Cycle 8:716-724 (2009). LKB1 has also been identified as a tumor suppressor gene that is lost in certain human malignancies, possibly reflecting its normal function as a suppressor of inappropriate proliferation. See, e.g., Shaw, R. J. Tumor suppression by LKB1: SIK-ness prevents metastasis. Sci. Signal. 2:55 (2009). However, it yet remains to be seen how frequently these two mechanisms of contact-mediated growth suppression are compromised in human cancers.

### Promotion of Malignancy by Corruption of the TGF-β Pathway

TGF-β is best known for its anti-proliferative effects, and the evasion of these effects by cancer cells is now known to be far more involved than just the simple shutdown of its signaling circuitry. See, e.g., Ikushima, H., Miyazono, K. TGFbeta signaling: a complex web in cancer progression. Nat. Rev. Cancer 10:415-424 (2010). In many late-stage tumors, TGF-β signaling is redirected away from suppressing cell proliferation and is found instead to activate a cellular program, termed the epithelial-to-mesenchymal transition (EMT), that confers on cancer cells traits associated with high-grade malignancy.

(v) Angiogenesis Induction

Tumors require sustenance in the form of nutrients and oxygen as well as an ability to evacuate metabolic wastes and carbon dioxide, just as normal, non-cancerous tissues do. The tumor-associated neovasculature (generated by the process of angiogenesis) addresses these metabolic needs. During embryogenesis, the development of the vasculature involves the generation of new endothelial cells and their subsequent assembly into tubes (i.e., vasculogenesis) in addition to the “sprouting” (i.e., angiogenesis) of new vessels from existing ones. Following this morphogenesis, normal vasculature becomes largely quiescent.

In the adult, as part of various physiologic processes (e.g., wound healing, female reproductive cycling, etc.) angiogenesis is transiently activated. However, in contrast, during tumor progression, an “angiogenic switch” is almost constantly activated, causing the normally quiescent vasculature to continually generate/sprout new vessels that assist in sustaining the expanding neoplastic growths. See, e.g., Baeriswyl, V., Christofori, G. The angiogenic switch in carcinogenesis. Semin. Cancer Biol. 19:329-337 (2009). A compelling body of evidence indicates that the angiogenic switch is governed by countervailing factors that either induce or oppose angiogenesis. Some of these angiogenic regulators include signaling proteins that bind to stimulatory or inhibitory cell surface receptors displayed by vascular endothelial cells. The well-known prototypes of angiogenesis inducers and inhibitors are vascular endothelial growth factor-A (VEGF-A) and thrombospondin-1 (TSP-1), respectively. The VEGF-A gene encodes ligands that are involved in: (i) orchestrating new blood vessel growth during embryonic and postnatal development; (ii) homeostatic survival of endothelial cells; and (iii) physiological and pathological situations in the adult. VEGF signaling via three receptor tyrosine kinases (VEGFR-1-3) is regulated at multiple levels, reflecting its complex functional purpose. For example, VEGF gene expression can be up-regulated both by hypoxia and by oncogene signaling. See, e.g., Ferrara, N. Pathways mediating VEGF-independent tumor angiogenesis. Cytokine Growth Factor Rev. 21:21-26 (2010). In addition, other pro-angiogenic signals, such as members of the fibroblast growth factor (FGF) family, have been implicated in sustaining tumor angiogenesis when their expression is chronically up-regulated. See, e.g., Baeriswyl, V., Christofori, G. The angiogenic switch in carcinogenesis. Semin. Cancer Biol. 19:329-337 (2009). The primary counter-balance in angiogenic switch is TSP-1, which also binds to transmembrane receptors displayed by endothelial cells; thereby evoking suppressive signals that can counteract pro-angiogenic stimuli. See, e.g., Kazerounian, S., Yee, K. O., Lawler, J. Thrombospondins in cancer. Cell. Mol. Life Sci. 65:700-712 (2008). The blood vessels produced within tumors by chronically activated angiogenesis and an unbalanced mix of proangiogenic signals are typically aberrant: tumor neovasculature is marked by precocious capillary sprouting, convoluted and excessive vessel branching, distorted and enlarged vessels, erratic blood flow, microhemorrhaging, leakiness, and abnormal levels of endothelial cell proliferation and apoptosis.

Angiogenesis is induced surprisingly early during the multistage development of invasive cancers both in animal models and in humans. Histological analyses of pre-malignant, noninvasive lesions, including dysplasias and in situ carcinomas arising in a variety of organs, have revealed the early tripping of the angiogenic switch. It had previously been thought that angiogenesis was only important when rapidly growing macroscopic tumors had formed. However, recent experimental data indicates that angiogenesis also contributes to the microscopic pre-malignant phase of neoplastic progression; thus further establishing its inclusion as a seminal biological capability of cancer.

### Angiogenic Switch Control

Tumors may exhibit diverse patterns of neo-vascularization once angiogenesis has been activated. By way of example, some tumors (e.g., highly aggressive pancreatic ductal adenocarcinomas) become hypovascularized and have been shown to comprise stromal “deserts” that are largely avascular and may even be actively anti-angiogenic. See, e.g., Olive, K. P., Jacobetz, M. A., et al., Inhibition of Hedgehog signaling enhances delivery of chemotherapy in a mouse model of pancreatic cancer. Science 324:1457-1461 (2009). Other tumors (e.g., human renal and pancreatic neuroendocrine carcinomas) are highly angiogenic and consequently possess dense vascularization. See, e.g., Zee, Y. K., O'Connor, J. P., Parker, et al., Imaging angiogenesis of genitourinary tumors. Nat. Rev. Urol. 7:69-82 (2010). Accordingly, these observations suggest that there is an initial “tripping” of the angiogenic switch during tumor development that is subsequently followed by ongoing neovascularization that is variable in intensity. This latter variability most probably being controlled by a complex biological “rheostat” that involves both the cancer cells and the associated stromal micro-environment. See, e.g., Baeriswyl, V., Christofori, G. The angiogenic switch in carcinogenesis. Semin. Cancer Biol. 19:329-337 (2009). It should be noted that the angiogenic switching mechanism can alter its form, even though the overall result is an inductive signal (e.g., VEGF). Further, in some tumors, dominant oncogenes operating within tumor cells (e.g., Ras and Myc) can upregulate expression of angiogenic factors; whereas in others, such inductive signals are produced indirectly by immune inflammatory cells.

### Endogenous Angiogenesis Inhibitors

A number of endogenous angiogenic regulators have been discovered. Most are proteins, and many are derived by proteolytic cleavage of structural proteins that are not themselves angiogenic regulators. These endogenous angiogeneic regulators include: TSP-1, angiostatin, and type 18 collagen (endostatin), and numerous other agents. See, e.g., Ribatti, D. Endogenous inhibitors of angiogenesis: a historical review. Leuk. Res. 33:638-644 (2009). Interestingly, a number of these endogenous inhibitors of angiogenesis have been detected in the circulation of normal mice and humans. This data suggest that such endogenous angiogenesis inhibitors serve under normal circumstances as physiologic regulators that modulate transitory angiogenesis during tissue remodeling and wound healing; whereas they may also act as intrinsic barriers to induction and/or persistence of angiogenesis by incipient neoplasias.

### Contribution of Bone Marrow-Derived Cells to Tumor Angiogenesis

A number of cell types originating in the bone marrow have been shown to play a crucial role in pathological angiogenesis, including cells of the innate immune system (e.g., macrophages, neutrophils, mast cells, and myeloid progenitors) that infiltrate pre-malignant lesions and progressed tumors and assemble at the margins of such lesions. These peri-tumoral inflammatory cells help to trip the angiogenic switch in previously quiescent tissue and to sustain ongoing angiogenesis associated with tumor growth and in facilitating local invasion. See, e.g., Ferrara, N. Pathways mediating VEGF-independent tumor angiogenesis. Cytokine Growth Factor Rev. 21:21-26 (2010).

(vi) Invasion and Metastatic Activation

Until relatively recently, the mechanisms underlying invasion and metastasis were largely enigmatic. What was clear was that as carcinomas arising from epithelial tissues progressed to higher pathological grades of malignancy (as reflected in local invasion and distant metastasis) the associated cancer cells typically developed alterations in their shape and their attachment both to other cells and to the extracellular matrix (ECM). Perhaps the best characterized of these alterations involved the loss by carcinoma cells of E-cadherin, a key cell-to-cell adhesion molecule. E-cadherin formed adherens junctions with adjacent epithelial cells, so as to assemble epithelial cell sheets and maintain the quiescence of the cells within these sheets. In accord, the increased expression of E-cadherin was well documented as an antagonist of invasion and metastasis; whereas the reduction of its expression was known to potentiate these aforementioned phenotypes. Moreover, the frequently observed down-regulation and occasional mutational inactivation of E-cadherin in human carcinomas also provided strong support for its role as a key suppressor of this seminal biological capability. See, e.g., Berx, G., van Roy, F. Involvement of members of the cadherin superfamily in cancer. Cold Spring Harb. Perspect. Biol. 1: a003129 (2009). The expression of genes encoding other cell-to-cell and cell-to-ECM adhesion molecules has also been shown to be altered in some highly aggressive carcinomas, with those genes favoring cytostasis typically being markedly downregulated. In contrast, adhesion molecules normally associated with the cellular migrations that occur during embryogenesis and inflammation are often upregulated (e.g., N-cadherin).

Invasion and metastasis has typically been envisioned as a sequence of discrete steps, often termed the “invasion-metastasis cascade” (see, e.g., Talmadge, J. E., Fidler, I. J. AACR centennial series: the biology of cancer metastasis: historical perspective. Cancer Res. 70:5649-5669 (2010)) which is depicted a as succession of cell-biologic changes, beginning with local invasion, then intravasation by cancer cells into nearby blood and lymphatic vessels, transit of cancer cells through the lymphatic and hematogenous systems, followed by escape of cancer cells from the lumina of such vessels into the parenchyma of distant tissues (extravasation), the formation of small nodules of cancer cells (micrometastases), and finally the growth of micrometastatic lesions into macroscopic tumors, this last step being termed “colonization”.

### Regulation of Invasion and Metastasis by EMT

A developmental regulatory program, referred to as the “epithelial-mesenchymal transition” (EMT), has become implicated as a means by which transformed epithelial cells can acquire the abilities to invade, to resist apoptosis, and to disseminate. See, e.g., Klymkowsky, M. W., Savagner, P. Epithelial-mesenchymal transition: a cancer researcher's conceptual friend and foe. Am. J. Pathol. 174:1588-1593 (2009). By co-opting a process involved in various steps of embryonic morphogenesis and wound healing, carcinoma cells can concomitantly acquire multiple attributes that enable invasion and metastasis. This multifaceted EMT program can be activated transiently or stably, and to differing degrees, by carcinoma cells during the course of invasion and metastasis.

A set of pleiotropically acting transcriptional factors direct the EMT and related migratory processes during embryogenesis and are expressed in various combinations in a number of malignant tumor types. Biological functions implicated in the processes of invasion and metastasis which are evoked by these transcriptional factors include: (i) a loss of adherens junctions and associated conversion from a polygonal/epithelial to a spindly/fibroblastic morphology, (ii) expression of matrix-degrading enzymes: (iii) increased motility; and (iv) heightened resistance to apoptosis. See, e.g., Taube, J. H., Herschkowitz, J. I., et al. Core epithelial-to-mesenchymal transition interactome gene-expression signature is associated with claudin-low and metaplastic breast cancer subtypes. Proc. Natl. Acad. Sci. USA 107:15449-15454 (2010). The available evidence suggests that these transcription factors regulate one another as well as overlapping sets of target genes and that heterotypic interactions of cancer cells with adjacent tumor-associated stromal cells can induce expression of the malignant cell phenotypes that are known to be choreographed by one or more of these transcriptional regulators. Moreover, cancer cells at the invasive margins of certain carcinomas can be seen to have undergone an EMT, suggesting that these cancer cells are subject to microenvironmental stimuli distinct from those received by cancer cells located in the cores of these lesions. See, e.g., Hlubek, F., Brabletz, T., et al., Heterogeneous expression of Wnt/beta-catenin target genes within colorectal cancer. Int. J. Cancer 121:1941-1948 (2007). In sum, although the evidence is still incomplete, it would appear that EMT-inducing transcription factors are able to orchestrate most steps of the invasion-metastasis cascade, with the exception of the final step of colonization.

### Contributions of Stromal Cells to Invasion and Metastasis

Recent evidence has demonstrated that there appears to be “cross-talk” between cancer cells and cells of the surrounding neoplastic stroma which is involved in the acquired capability for invasive growth and metastasis. See, e.g., Egeblad, M., Nakasone, E. S., Werb, Z. Tumors as organs: complex tissues that interface with the entire organism. Dev. Cell 18:884-901 (2010). By way of example, mesenchymal stem cells (MSCs) present in the tumor stroma have been found to secrete CCL5/RANTES in response to signals released by cancer cells; with the CCL5 then acting reciprocally on the cancer cells to stimulate invasive behavior. See, e.g., Karnoub, A. E., Dash, et al., Mesenchymal stem cells within tumour stroma promote breast cancer metastasis. Nature 449:557-563 (2007). In addition, macrophages at the tumor periphery have been shown to foster local invasion by supplying matrix-degrading enzymes such as metalloproteinases and cysteine cathepsin proteases, and in one model system, the invasion promoting macrophages were activated by IL-4 produced by the cancer cells. See, e.g., Gocheva, V., Wang, H. W., et al., IL-4 induces cathepsin protease activity in tumor-associated macrophages to promote cancer growth and invasion. Genes Dev. 24:241-255 (2010). The aforementioned observations tend to indicate that the phenotypes of high grade malignancy do not arise in a strictly cell-autonomous manner, and that their manifestation cannot be understood solely through analyses of tumor cell genomes.

### Different Cancer Types May Have Distinct Forms of Invasion

The EMT program regulates a particular type of invasiveness that has been termed “mesenchymal”. In addition, two other distinct modes of invasion have been identified and implicated in cancer cell invasion. See, e.g., Madsen, C. D., Sahai, E. Cancer dissemination—Lessons from leukocytes. Dev. Cell 19:13-26 (2010). The first, “collective invasion” involves nodules of cancer cells advancing en masse into adjacent tissues and is characteristic of, e.g., squamous cell carcinomas; interestingly, such cancers are rarely metastatic, suggesting that this form of invasion lacks certain functional attributes that facilitate metastasis. The second, is an “amoeboid” form of invasion, in which individual cancer cells show morphological plasticity, enabling them to slither through existing interstices in the extracellular matrix rather than clearing a path for themselves, as occurs in both the mesenchymal and collective forms of invasion.

Another emerging concept, involves the facilitation of cancer cell invasion by inflammatory cells that assemble at the boundaries of tumors, producing the extracellular matrix-degrading enzymes and other factors that enable invasive growth (see, e.g., Kessenbrock, K., Plaks, V., Werb, Z. Matrix metalloproteinases: Regulators of the tumor microenvironment. Cell 141:52-67 (2010)); these functions may obviate the need of cancer cells to produce these proteins through activation of EMT programs. Accordingly, cancer cells may secrete the chemoattractants that recruit the proinvasive inflammatory cells rather than producing the matrix-degrading enzymes themselves.

### The Complexity of Metastatic Colonization

Metastasis can be broken down into two major phases: (i) the physical dissemination of cancer cells from the primary tumor to distant tissues, and (ii) the adaptation of these cells to foreign tissue microenvironments that results in successful colonization (i.e., the growth of micrometastases into macroscopic tumors). The multiple steps of dissemination would seem to be under the aegis of the EMT and similarly acting migratory programs. It must be noted, however, that colonization is not strictly coupled with physical dissemination, as evidenced in many patients by the presence of a plethora of micrometastases that have successfully disseminated, but never progress to macroscopic metastatic tumors. See, e.g., Talmadge, J. E., Fidler, I. J. AACR centennial series: the biology of cancer metastasis: historical perspective. Cancer Res. 70:5649-5669 (2010).

By way of example, in some types of cancer, the primary tumor may release systemic suppressor factors that render such micrometastases dormant, as revealed clinically by explosive metastatic growth soon after resection of the primary growth. Conversely, in other cancer (e.g., breast cancer and melanoma), macroscopic metastases may erupt decades after a primary tumor has been surgically removed or pharmacologically destroyed. See, e.g., Barkan, D., Green, J. E., Chambers, A. F. Extracellular matrix: a gatekeeper in the transition from dormancy to metastatic growth. Eur. J. Cancer 46:1181-1188 (2010). One can possibly infer from such findings that these micrometastases may lack various seminal biological capabilities which are necessary for vigorous growth (e.g., the ability to activate angiogenesis), as the inability of certain experimentally generated dormant micrometastases to form macroscopic tumors has been ascribed to their failure to activate tumor angiogenesis. See, e.g., Naumov, G. N., Folkman, J., Straume, O., Akslen, L. A. Tumorvascular interactions and tumor dormancy. APMIS 116:569-585 (2008). Recent experiments have also shown that nutrient starvation can induce intense autophagy that causes cancer cells to shrink and adopt a state of reversible dormancy; such cells may exit this state and resume active growth and proliferation when changes in tissue micro-environment (e.g., access to more nutrients) permit. See, e.g., Kenific, C. M., Thorburn, A., Debnath, J. Autophagy and metastasis: another double-edged sword. Curr. Opin. Cell Biol. 22:241-245 (2010). Other mechanisms of micro-metastatic dormancy may involve anti-growth signals embedded in normal tissue extracellular matrix (see, e.g., Barkan, D., Green, J. E., Chambers, A. F. Extracellular matrix: a gatekeeper in the transition from dormancy to metastatic growth. Eur. J. Cancer 46:1181-1188 (2010)) and tumor-suppressing actions of the immune system (see, e.g., Teng, M. W. L., Swann, J. B., Koebel, et al. Immune-mediated dormancy: an equilibrium with cancer. J. Leukoc. Biol. 84:988-993 (2008)).

Most disseminated cancer cells are likely to be poorly adapted, at least initially, to the microenvironment of the tissue in which they have landed. Accordingly, each type of disseminated cancer cell may need to develop its own set of ad hoc solutions to the problem of thriving in the microenvironment of one or another foreign tissue. These adaptations might require hundreds of distinct colonization programs, each dictated by the type of disseminating cancer cell and the nature of the tissue microenvironment in which colonization is proceeding. However, certain tissue microenvironments may be preordained to be intrinsically hospitable to disseminated cancer cells. See, e.g., Talmadge, J. E., Fidler, I. J. AACR centennial series: the biology of cancer metastasis: historical perspective. Cancer Res. 70:5649-5669 (2010).

Metastatic dissemination has long been depicted as the last step in multistep primary tumor progression, and indeed for many tumors that is likely the case, as illustrated by recent genome sequencing studies that present genetic evidence for clonal evolution of pancreatic ductal adenocarcinoma to metastasis. See, e.g., Yachida, S., Jones, S., et al., Distant metastasis occurs late during the genetic evolution of pancreatic cancer. Nature 467:1114-1117 (2010). Conversely, recent findings indicate that cells can disseminate remarkably early, dispersing from ostensibly noninvasive premalignant lesions in both mice and humans. See, e.g., Coghlin, C., Murray, G. I. Current and emerging concepts in tumour metastasis. J. Pathol. 222:1-15 (2010).

Although cancer cells can clearly disseminate from such pre-neoplastic lesions and seed the bone marrow and other tissues, their capability to colonize these sites and develop into pathologically significant macrometastases remains to be proven. Early metastatic dissemination is viewed as a demonstrable phenomenon in mice and humans whose clinical significance is yet to be established. Beyond the timing of their dissemination, it also remains unclear when and where cancer cells develop the ability to colonize foreign tissues as macroscopic tumors. This capability may arise during primary tumor formation as a result of a tumor's particular developmental path prior to any dissemination, such that primary tumor cells entering the circulation are fortuitously endowed with the ability to colonize certain distant tissue sites. See, e.g., Talmadge, J. E., Fidler, I. J. AACR centennial series: the biology of cancer metastasis: historical perspective. Cancer Res. 70:5649-5669 (2010). Alternatively, the ability to colonize specific tissues may only develop in response to the selective pressure on already disseminated cancer cells to adapt to growth in foreign tissue microenvironments.

Once the cells within metastatic colonies have developed such tissue-specific colonizing ability, they may proceed to disseminate further, not only to new sites within the body but also back to the primary tumors in which their “ancestors” arose. Accordingly, in yet another example of heterogeneity, tissues-specific colonization programs that are evident among cells within a primary tumor may originate not from classical tumor progression occurring within the primary lesion but instead from emigrants that have returned home. Such reseeding is consistent with the aforementioned studies of human pancreatic cancer metastasis. See, e.g., Yachida, S., Jones, S., et al., Distant metastasis occurs late during the genetic evolution of pancreatic cancer. Nature 467:1114-1117 (2010). Furthermore, substantial progress is also being made in defining sets of genes (i.e., the “metastatic signatures”) that correlate with and appear to facilitate the establishment of macroscopic metastases in specific tissues. See, e.g., Coghlin, C., Murray, G. I. Current and emerging concepts in tumour metastasis. J. Pathol. 222:1-15 (2010). Colonization is unlikely to depend exclusively upon cell-autonomous processes. Instead, it almost certainly requires the establishment of a permissive tumor micro-environment composed of critical stromal support cells. For these reasons, the process of colonization is likely to encompass a large number of cell biological programs that are, in aggregate, considerably complex and diverse.

(vii) Energy Metabolism Reprogramming

The chronic and frequently uncontrolled cell proliferation that represents the essence of neoplastic disease involves not only deregulated control of cell proliferation but also corresponding adjustments of energy metabolism in order to fuel cell growth and division. Under aerobic conditions, normal cells process glucose, first to pyruvate via glycolysis in the cytosol and thereafter to carbon dioxide in the mitochondria (i.e., mitochondrial oxidative phosphorylation). In contrast, under anaerobic conditions, glycolysis is favored and relatively little pyruvate is dispatched to the oxygen-consuming mitochondria. One anomalous characteristic of cancer cell energy metabolism is that cancer cells (even in the presence of oxygen) can reprogram their glucose metabolism, and thus their energy production, by limiting their energy metabolism largely to glycolysis, leading to a state that has been termed “aerobic glycolysis”. Such reprogramming of energy metabolism is seemingly counter-intuitive, as the cancer cells must compensate for the 18-fold lower efficiency of ATP production afforded by glycolysis relative to mitochondrial oxidative phosphorylation. The cancer cells compensate, in part, by upregulating glucose transporters (e.g., GLUT1) which substantially increases glucose import into the cytoplasm. See, e.g., Jones, R. G., Thompson, C. B. Tumor suppressors and cell metabolism: a recipe for cancer growth. Genes Dev. 23:537-548 (2009). Markedly increased uptake and utilization of glucose have been documented in many human tumor types, by visualizing glucose uptake using positron emission tomography (PET) with a radiolabeled analog of glucose (18F-fluorodeoxyglucose; FDG) as a reporter molecule.

Glycolytic fueling has been shown to be associated with activated oncogenes (e.g., RAS, MYC) and mutant tumor suppressors (e.g., TP53), whose alterations in tumor cells have been selected primarily for their benefits in conferring the seminal biological capabilities of cell proliferation, avoidance of cytostatic controls, and attenuation of apoptosis. This reliance on glycolysis can be further accentuated under the hypoxic conditions that operate within many tumors; wherein the hypoxia response system acts pleiotropically to upregulate glucose transporters and multiple enzymes of the glycolytic pathway. See, e.g., Semenza, G. L. HIF-1: upstream and downstream of cancer metabolism. Curr. Opin. Genet. Dev. 20:51-56 (2010). Thus, both the Ras oncoprotein and hypoxia can independently increase the levels of the HIF1a and HIF2a transcription factors, which in turn upregulate glycolysis. See, e.g., Id.

A quantitative functional rationale for this glycolytic switch in cancer cells has not yet been elucidated. However, one recently revived and refined hypothesis holds that increased glycolysis allows shunting of glycolytic intermediates into numerous biosynthetic pathways (e.g., those generating nucleosides and amino acids) which facilitates the biosynthesis of the macromolecules and organelles required for assembling new (cancer) cells. This is known as the “Warburg effect”. See, e.g., Vander Heiden, M. G., Cantley, L. C., Thompson, C. B. Understanding the Warburg effect: the metabolic requirements of cell proliferation. Science 324:1029-1033 (2009). Moreover, Warburg-like metabolism seems to be present in many rapidly dividing embryonic tissues, once again suggesting a role in supporting the large-scale biosynthetic programs that are required for active cell proliferation. Interestingly, some tumors have been found to contain two distinct sub-populations of cancer cells that differ in their energy-generating pathways. One sub-population consists of glucose-dependent (i.e., “Warburg effect) cells that secrete lactate; whereas cells of the second sub-population preferentially import and utilize the lactate produced by their neighbors as their main energy source, employing part of the citric acid cycle to do so. See, e.g., Kennedy, K. M., Dewhirst, M. W. Tumor metabolism of lactate: the influence and therapeutic potential for MCT and CD147 regulation. Future Oncol. 6:127-148 (2010). These two cell populations evidently function symbiotically—the hypoxic cancer cells depend on glucose for fuel and secrete lactate as waste, which is subsequently imported and preferentially used as fuel by their better-oxygenated brethren.

Altered energy metabolism is proving to be widespread in many types of cancer cells and is becoming recognized as one of the seminal biological capabilities. The redirection of energy metabolism is largely orchestrated by proteins that are involved in various ways in programming the other enumerated seminal biological capabilities. When viewed in this manner, aerobic glycolysis is simply another phenotype that is programmed by proliferation-inducing oncogenes. Interestingly, activating (i.e., gain-of-function) mutations in the isocitrate dehydrogenase (IDH) enzymes have been reported in glioma and other human tumors. Although these mutations may prove to have been clonally selected for their ability to alter energy metabolism, there is confounding data associating their activity with elevated oxidation and stability of the HIF-1 transcription factors, which could in turn affect genome stability and angiogenesis/invasion, respectively, thus blurring the lines of phenotypic demarcation. See, e.g., Reitman, Z. J., Yan, H. Isocitrate dehydrogenase 1 and 2 mutations in cancer: alterations at a crossroads of cellular metabolism. J. Natl. Cancer Inst. 102:932-941 (2010).

(viii) Evading Immune Destruction

An additional unresolved issue surrounding tumor formation involves the role that the immune system plays in resisting or eradicating formation and progression of incipient neoplasias, late-stage tumors, and micrometastases. The long-standing theory of immune surveillance proposes that cells and tissues are constantly monitored by an ever-alert immune system, and that such immune surveillance is responsible for recognizing and eliminating the vast majority of incipient cancer cells and thus nascent tumors. According to this logic, solid tumors that do appear have somehow managed to avoid detection by the various arms of the immune system or have been able to limit the extent of immunological killing, thereby evading eradication. The role of defective immunological monitoring of tumors would seem to be validated by the striking increases of certain cancers in immunocompromised individuals. However, the great majority of these are virus-induced cancers, suggesting that much of the control of this class of cancers normally depends on reducing viral burden in infected individuals, in part through eliminating virus-infected cells. These observations, therefore, seem to shed little light on the possible role of the immune system in limiting formation of the >80% of tumors of nonviral etiology.

In recent years, however, an increasing body of evidence, both from genetically engineered mice and from clinical epidemiology, suggests that the immune system operates as a significant barrier to tumor formation and progression, at least in some forms of non-virally-induced cancer. When mice genetically engineered to be deficient for various components of the immune system were assessed for the development of carcinogen-induced tumors, it was observed that tumors arose more frequently and/or grew more rapidly in the immunodeficient mice relative to immunocompetent controls. In particular, deficiencies in the development or function of CD8+ cytotoxic T lymphocytes (CTLs), CD4+ Th1 helper T cells, or natural killer (NK) cells each led to demonstrable increases in tumor incidence; moreover, mice with combined immunodeficiencies in both T cells and NK cells were even more susceptible to cancer development. The results indicated that, at least in certain experimental models, both the innate and adaptive cellular arms of the immune system are able to contribute significantly to immune surveillance and thus tumor eradication. See, e.g., Teng, M. W. L., Swann, J. B., et al., Immune-mediated dormancy: an equilibrium with cancer. J. Leukoc. Biol. 84:988-993 (2008). Interestingly, transplantation experiments have shown that cancer cells that originally arose in immunodeficient mice are often markedly inefficient at initiating secondary tumors in syngeneic immunocompetent hosts, whereas cancer cells from tumors arising in immunocompetent mice are equally efficient at initiating transplanted tumors in both types of hosts. Id.

These aforementioned experimental results may be interpreted as follows:
(i) highly immunogenic cancer cell clones are routinely eliminated in immunocompetent hosts (in a process that has been referred to as “immunoediting”) leaving behind only weakly immunogenic variants to replicate and generate solid tumors; such weakly immunogenic cells can thereafter colonize both immunodeficient and immunocompetent hosts; and
(ii) when arising in immunodeficient hosts, the immunogenic cancer cells are not selectively depleted and can, instead, prosper along with their weakly immunogenic counterparts; when cells from such nonedited tumors are serially transplanted into syngeneic recipients, the immunogenic cancer cells are rejected when they confront, for the first time, the competent immune systems of their secondary hosts.
   Clinical epidemiology also increasingly supports the existence of anti-tumoral immune responses in some forms of human cancer. See, e.g., Bindea, G., Mlecnik, B., et al., Natural immunity to cancer in humans. Curr. Opin. Immunol. 22:215-222 (2010). By way of example, patients with colon and ovarian tumors that are heavily infiltrated with CTLs and NK cells have a better prognosis than those that lack such abundant killer lymphocytes (see, e.g., Pages, F., Galon, J., et al., Immune infiltration in human tumors: a prognostic factor that should not be ignored. Oncogene 29:1093-1102 (2010)); the case for other types of cancers is less compelling and is the subject of ongoing investigations.

The epidemiology of chronically immunosuppressed patients does not indicate significantly increased incidences of the major forms of non-viral human cancer. This finding may be interpreted as an argument against the importance of immune surveillance as an effective barrier to tumorigenesis and tumor progression. However, it must be noted that HIV and pharmacologically-immunosuppressed patients are predominantly immunodeficient in the T- and B-cell compartments. Accordingly, these patients do not present with the complex multi-component immunological deficiencies that have been produced in, e.g., genetically engineered mutant mice lacking both NK cells and CTLs; thus leaving open the possibility that such patients still have residual capability for mounting an effective immunological defense against cancer through the actions of NK and other innate immune cells. Moreover, the aforementioned discussions of cancer immunology may greatly oversimplify tumor-host immunological interactions, as highly immunogenic cancer cells may well evade immune destruction by disabling components of the immune system that have been dispatched to eliminate them. For example, cancer cells may “paralyze” infiltrating CTLs and NK cells, by secreting TGF-β or other immunosuppressive factors. See, e.g., Yang, L., Pang, Y., Moses, H. L. TGF-beta and immune cells: an important regulatory axis in the tumor microenvironment and progression. Trends Immunol. 31:220-227 (2010). Additional, more subtle, mechanisms may also operate through the recruitment of inflammatory cells that are actively immunosuppressive, including regulatory T cells (Tregs) and myeloid-derived suppressor cells (MDSCs). Both cells can suppress the actions of cytotoxic lymphocytes. See, e.g., Mougiakakos, D., Choudhury, A., et al., Regulatory T cells in cancer. Adv. Cancer Res. 107:57-117 (2010).

In summary, the multiple characteristics, capabilities, attributes, and manifestations set forth on the previous pages further reflect the heterogeneous nature of cancer.

### IV. Multiple Molecular Targets of the Present Invention

Discussed in this section are important molecular targets of the present invention (hereinafter “target molecules”). As described below, the sulfur-containing, amino acid-specific small molecules of the present invention possess the ability to contemperaneously or simultaneously modify and/or modulate multiple target molecules.

A. Tyrosine Kinases

The term kinase describes a large family of enzymes that are responsible for catalyzing the transfer of a phosphoryl group from a nucleoside triphosphate donor, such as ATP, to an acceptor molecule. Tyrosine kinases catalyze the phosphorylation of tyrosine residues in proteins. The phosphorylation of tyrosine residues, in turn, causes a change in the function of the protein that they are contained in. Phosphorylation at tyrosine residues controls a wide range of properties in proteins such as enzyme activity, subcellular localization, and interaction between molecules.

(i) c-MET

The MET proto-oncogene encodes for the receptor tyrosine kinase (RTK), c-MET. MET encodes a protein known as hepatocyte growth factor receptor (HGFR). The hepatocyte growth factor receptor protein possesses tyrosine kinase activity. See, e.g., Cooper, C. S., The MET oncogene: from detection by transfection to transmembrane receptor for hepatocyte growth factor. Oncogene 7(1):3-7 (1992). c-MET is a membrane receptor that is essential for embryonic development and tissue repair (e.g., wound healing). Hepatocyte growth factor (HGF) is the only known ligand of the c-MET receptor. MET is normally expressed in cells of epithelial origin, although it has also been identified in endothelial cells, neurons, hepatocytes, hematopoietic cells, and melanocytes. Expression of HGF is generally restricted to cells of mesenchymal origin, although some epithelial cancer cells appear to express both HGF and MET.

The MET proto-oncogene has a total length of 125,982 bp and is located in the 7q31 locus of chromosome 7. MET is transcribed into a 6,641 bp mature mRNA which is then translated into a 1,390 amino acid residue c-MET protein. c-MET is a receptor tyrosine kinase that is produced as a primary single-chain precursor protein that is post-translationally proteolytically cleaved at a furin site to yield a highly glycosylated extracellular α-subunit and a transmembrane β-subunit, which are then covalently linked via a disulfide bond to form the mature receptor. Under normal conditions, c-MET dimerizes and autophosphorylates upon ligand binding, which in turn creates active docking sites for proteins that mediate downstream signaling leading to the activation/modulation of a variety of proteins. Such activation/modulation evokes a variety of pleiotropic biological responses leading to increased cell growth, scattering and motility, invasion, protection from apoptosis, branching morphogenesis, and angiogenesis. However, under pathological conditions improper activation of c-MET may confer proliferative, survival and invasive/metastatic abilities of cancer cells.

Over the years many groups have established that c-MET and HGF are highly expressed in a large number of solid and soft tumors (for a comprehensive list, see www.vai.org/met). The list of tumors in which c-MET is expressed is quite large, and it has been shown that high levels of c-MET can lead to the constitutive activation of the enzyme, as well as rendering cells sensitive to subthreshold amounts of HGF. Although many of these studies have not identified the level of c-MET receptor activity/phosphorylation or compared the expression level with its normal counterpart, it could be speculated that it is expressed with autocrine loops of HGF/c-MET which increase cell proliferation and metastases. See, e.g., Navab, R., Liu, J., et al. Co-overexpression of Met and hepatocyte growth factor promotes systemic metastasis in NCI-H460 non-small cell lung carcinoma cells. Neoplasia 11:1292-1300 (2009). Furthermore, independent studies have also shown that HGF is expressed ubiquitously throughout the body, showing this growth factor to be a systemically available cytokine as well as coming from the tumor stroma. See, e.g., Vuononvirta, R., Sebire, N.J., et al. Expression of hepatocyte growth factor and its receptor met in Wilms' tumors and nephrogenic rests reflects their roles in kidney development. Clin. Cancer Res. 15:2723-2730 (2009). A positive paracrine and autocrine loop of c-MET activation can therefore lead to further MET expression.

c-MET was first identified as the product of a chromosomal rearrangement after treatment with the carcinogen N-methyl-NO-nitro-N-nitrosoguanidine, See, e.g., Cooper, C. S., Park, M., et al., Molecular cloning of a new transforming gene from a chemically transformed human cell line. Nature 311:29-33 (1984). This rearrangement results in a constitutively fused oncogene (TPR-MET) which is translated into an oncoprotein following dimerization by a leucine-zipper motif located in the TPR moiety. This provides the structural requirement for c-MET kinase to be constitutively active. TPR-MET has been shown to possess the ability to transform epithelial cells and to induce spontaneous mammary tumors when ubiquitously over-expressed in transgenic mice. These findings set the starting point for a currently ongoing effort to unveil all oncogenic abilities of c-MET. It took more than a decade to provide the proof of concept for the role of c-MET in human cancers, which became evident following the identification of activating point mutations in the germline of patients affected by hereditary papillary renal carcinomas. See, e.g., Schmidt, L., Junker, K., et al., Novel mutations of the MET proto-oncogene in papillary renal carcinomas. Oncogene 18:2343-2350 (1999). A large number of reports have shown that an altered level of receptor tyrosine kinase (RTK) activation can play an important role in the pathophysiology of cancer. See, e.g., Lemmon, M. A. and Schlessinger, J. Cell signaling by receptor tyrosine kinases. Cell 141:1117-1134 (2010). Deregulation and the consequent aberrant signaling of c-MET may occur by different mechanisms including gene amplification, overexpression, activating mutations, increased autocrine or paracrine ligand-mediated stimulation, and interaction with other active cell-surface receptors.

Many studies have reported that c-MET is overexpressed in a variety of carcinomas including lung, breast, ovary, kidney, colon, thyroid, liver, and gastric carcinomas. See, e.g., Knowles, L. M., Stabile, L. P., et al. HGF and c-Met participate in paracrine tumorigenic pathways in head and neck squamous cell cancer. Clin. Cancer Res. 15:3740-3750 (2009). Such over-expression could be the result of transcriptional activation, hypoxia-induced over-expression, or as a result of MET amplification. See, e.g., Cappuzzo, F., Marchetti, A., et al. Increased MET gene copy number negatively affects survival of surgically resected non-small-cell lung cancer patients. J. Clin. Oncol. 27:1667-1674 (2009); Cappuzzo, F., Janne, P. A., et al. MET increased gene copy number and primary resistance to gefitinib therapy in non-small-cell lung cancer patients. Ann. Oncol. 20:298-304 (2009). In addition, transgenic mice overexpressing c-MET have been reported to spontaneously develop hepatocellular carcinoma, and when the transgene was inactivated, tumor regression was reported even in large tumors. See, e.g., Wang, R., Ferrell, L. D., et al. Activation of the Met receptor by cell attachment induces and sustains hepatocellular carcinomas in transgenic mice. J. Cell. Biol. 153:1023-1034 (2001).

Abnormal MET activation in cancer correlates with poor prognosis, where aberrantly active MET triggers tumor growth, formation of new blood vessels (angiogenesis) that supply the tumor with nutrients, and cancer spread to other organs (metastasis). MET is deregulated in many types of human malignancies, including cancers of the: kidney, liver, stomach, breast, and brain. Normally, only stem cells and progenitor cells express MET, which allows these cells to grow invasively in order to generate new tissues in an embryo or regenerate damaged tissues in an adult. However, cancer stem cells are thought to hijack the ability of normal stem cells to express MET, and thus become the cause of cancer persistence and spread to other sites in the body.

(ii) Anaplastic Lymphoma Kinase (ALK)

Anaplastic lymphoma kinase (ALK) also known as ALK tyrosine kinase receptor or CD246 (cluster of differentiation 246) is an enzyme that in humans is encoded by the ALK gene. See, e.g., Cui, J. J.; Tran-Dube, M.; et al., Structure Based Drug Design of Crizotinib (PF-02341066), a Potent and Selective Dual Inhibitor of Mesenchymal-Epithelial Transition Factor (c-MET) Kinase and Anaplastic Lymphoma Kinase (ALK). J. Med. Chem. 54:6342-6363 (2011).

ALK belongs to the tyrosine kinase receptor family. By homology, ALK is most similar to leukocyte tyrosine kinase, and both belong to the insulin-receptor superfamily. ALK is a single-chain transmembrane receptor comprising three structural domains. The extracellular domain contains an N-terminal signal peptide sequence and is the ligand-binding site for the putative activating ligands of ALK (i.e., pleiotrophin and midkine). This is followed by the transmembrane and juxtamembrane region which contains a binding site for phosphotyrosine-dependent interaction with insulin receptor substrate-1. The final section has an intracellular tyrosine kinase domain with three phosphorylation sites (Y1278, Y1282, and Y1283), followed by the C-terminal domain with interaction sites for phospholipase C-γ and Src homology 2 domain containing SHC. These sequences are absent in the product of the transforming ALK gene. Under physiologic conditions, binding of a ligand induces homodimerization of ALK, leading to trans-phosphorylation and kinase activation. In ALK translocations, the 5′-terminus fusion partners provide dimerization domains, enabling ligand-independent activation of the kinase. In addition, unlike native ALK, which localizes to the plasma membrane, the majority of ALK fusion proteins localize to the cytoplasm. This difference in cellular localization may also contribute to deregulated ALK activation.

The EML4-ALK fusion oncogene represents one of the newest molecular targets in cancer (especially in non-small cell lung carcinoma (NSCLC)). EML4-ALK was identified by the screening of a cDNA library derived from a the tumor of a NSCLC (adenocarcinoma) of the lung. See, e.g., Soda, M., Choi, Y. L, et al. Identification of the transforming EML4-ALK fusion gene in non-small cell lung cancer. Nature 448:561-566 (2007). This fusion arises from an inversion on the short arm of chromosome 2 [Inv (2) (p21p23)] that joins exons 1-13 of echinoderm microtubule associated protein-like 4 (EML4) to exons 20-29 of ALK. The resulting chimeric protein, EML4-ALK, contains an N-terminus derived from EML4 and a C-terminus containing the entire intracellular tyrosine kinase domain of ALK. Since the initial discovery of this fusion, multiple other variants of EML-ALK have been reported, all of which encode the same cytoplasmic portion of ALK but contain different truncations of EML4. See, e.g., Choi, Y. L., Takeuchi, K., et al. Identification of novel isoforms of the EML4-ALK transforming gene in non-small cell lung cancer. Cancer Res. 68:4971-4976 (2008). In addition, fusions of ALK with other partners including TRK-fused gene (TFG) and KIF5B have also been described in lung cancer, but seem to be much less common than EML4-ALK. See, e.g., Rikova, K., Guo, A., et al. Global survey of phosphotyrosine signaling identifies oncogenic kinases in lung cancer. Cell 131:1190-1203 (2007).

Chromosomal aberrations involving ALK have been identified in several other cancers, including anaplastic large cell lymphomas (ALCL), inflammatory myofibroblastic tumors (IMT), and neuroblastomas. See, e.g., Chiarle, R., Voena, C., et al. The anaplastic lymphoma kinase in the pathogenesis of cancer. Nat. Rev. Cancer 8:11-23 (2008). In cases of ALK translocation, including EML4-ALK, the fusion partner has been shown to mediate ligand-independent dimerization of ALK, resulting in constitutive kinase activity. In cell culture systems, EML4-ALK possesses potent oncogenic activity. In transgenic mouse models, lung-specific expression of EML4-ALK leads to the development of numerous lung adenocarcinomas. See, e.g., Soda, M., Takada, S., et al. A mouse model for EML4-ALK-positive lung cancer. Proc. Natl. Acad. Sci. U.S.A. 105:19893-19897 (2008). Cancer cell lines harboring the EML4-ALK translocation can be effectively inhibited by small molecule inhibitors targeting ALK. See, e.g., Koivunen, J. P., Mermel, C., et al. EML4-ALK fusion gene and efficacy of an ALK kinase inhibitor in lung cancer. Clin. Cancer Res. 14:4275-4283 (2008). Treatment of EML4-ALK transgenic mice with ALK inhibitors also results in tumor regression. Taken together, these aforementioned results support the notion that ALK-driven lung cancers are dependent upon the fusion oncogene.

The best characterized alterations of ALK associated with cancer are gene rearrangements; these have been observed in hematologic as well as in non-hematologic malignancies. The role of ALK in cancer was first identified as part of the NPM-ALK gene fusion involved in the pathogenesis of a subset of anaplastic large cell lymphoma (ALCL; see, e.g., Li, S. Anaplastic lymphoma kinase-positive large B-cell lymphoma: a distinct clinicopathological entity. Int. J. Clin. Exp. Pathol. 2:508-518 (2009)). Subsequently, multiple fusion partners forming ALK chimeric proteins in this disease have also been identified. ALK rearrangements have also been reported in other lymphomas, such as diffuse large B-cell lymphomas (DLBCL). In solid tumors, ALK translocations were first described in inflammatory myofibroblastic tumors (IMT).

Point mutations have been found in 6-8% of primary neuroblastomas. Germ-line mutations have been identified in families with more than one sibling with neuroblastoma. Somatic mutations with wild-type ALK in matched constitutional DNAs have also been described in non-familial neuroblastoma cases. These mutations are located mainly in the TK domain; the most frequent being the gain-of-function mutations F1174L and R1275Q. These mutations are associated with increased expression, phosphorylation, and kinase activity of the ALK protein. Further, they have been shown to have Ba/F3 cell-transforming capacity. In some cases, these mutations coexist with an increased copy number of the ALK gene. See, e.g., Janoueix-Lerosey, I., Lequin, D., et al. Somatic and germline activating mutations of the ALK kinase receptor in neuroblastoma. Nature 455:967-970 (2008). Interestingly, these mutations (particularly the F1174L) are predictive of response (as indicated by increased apoptosis and inhibition of growth) to short hairpin ALK-specific knockdown and TK ALK inhibitors (TAE684 and PF-12341066). Notably, protein expression levels in ALK mutant neuroblastoma models do not directly correlate with sensitivity to ALK inhibitors. It seems that this finding could be explained by the existence of a higher turnover rate of the ALK protein in cells with constitutively activated ALK.

An increased copy number of ALK has also been described in neuroblastoma cell lines and tumors, which can coexist with ALK gene mutation. In this disease, amplification, as well as mutation of ALK, has been associated with MYCN amplification, the most frequent amplicon in neuroblastoma defining a high-risk subgroup of patients that may benefit from ALK-selective inhibition. See, e.g., Janoueix-Lerosey, I., Lequin, D., et al. Somatic and germline activating mutations of the ALK kinase receptor in neuroblastoma. Nature 455:967-970 (2008).

In addition, a number of research groups have described ALK gene amplification in non-small cell lung cancer (NSCLC) tissue. See, e.g., Perner, S., Wagner, P. L., et al. EML4-ALK fusion lung cancer. Neoplasia 10:298-302 (2008); Salido, M., Pijuan, L., et al. Increased ALK gene copy number and amplification are frequent in non-small cell lung cancer. J. Thorac. Oncol. 6:21-27 (2011); Grande, E., Bolós, M. V., Arriola, E. Targeting Oncogenic ALK: A Promising Strategy for Cancer Treatment. Mol. Cancer Ther. 10:569-579 (2011). A recent study showed a relatively high frequency of copy number of mainly low level gains (60%), and amplification (10%); wherein the pattern of amplification, in the majority of NSCLC cases, was found to be characterized by a small percentage of cells within the tumor harboring this amplification. See, Grande, E., Bolos, M. V., Arriola, E. Targeting Oncogenic ALK: A Promising Strategy for Cancer Treatment. Mol. Cancer Ther. 10:569-579 (2011). However, it was found that some cases had >40% of cells with ALK amplification.

(iii) c-ROS

The c-ROS gene was first discovered in 1986 when a recombinant DNA clone containing cellular sequences homologous to the transforming sequence, v-ROS, of the avian sarcoma virus UR29-11 was isolated from a chicken genomic DNA library. UR2 sarcoma virus is a retrovirus of chicken that encodes for a fusion protein, P68^{gag-ROS}, having tyrosine-specific kinase activity. See, e.g., Feldman, R. A., Wang, L. H., et al. Avian sarcoma virus UR2 encodes a transforming protein which is associated with a unique protein kinase activity. J. Virol. 42:228-236 (1982). The oncogene, v-ROS, of UR2 carries a kinase domain that is homologous to those present in the oncogenes of the src family. The c-ROS sequence appeared to be conserved in vertebrate species, from fish to mammals (including humans). The comparison of the deduced amino acid sequence of c-ROS and that of v-ROS showed two differences: (i) v-ROS contains three amino acids insertion within the hydrophobic domain (TM domain), presumed to be involved in membrane association; and (ii) the twelve carboxy-terminal amino acids of v-ROS are completely different from those of the deduced c-ROS sequence. See, e.g., Neckameyer, W. S., Shibuya, M., Hsu, M. T., Wang, L. H. Proto-oncogene c-ROS codes for a molecule with structural features common to those of growth factor receptors and displays tissue-specific and developmentally regulated expression. Mol. Cell Biol. 6:1478-1486 (1986).

The human c-ROS gene was mapped to the human chromosome 6, region 6q16-6q22. This region of chromosome 6 is involved in nonrandom chromosomal rearrangement in specific neoplasias, including: acute lymphoplastic leukemia, malignant melanoma, and ovarian carcinomas. c-ROS gene over-expression and/or mutations were found mainly in brain and lung cancers, in addition to chemically-induced stomach cancer, breast fibroadenomas, liver cancer, colon cancer, and kidney cancer.

### ROS in Non-Small Cell Lung Cancer (NSCLC)

In a large-scale survey of tyrosine kinase activity in lung cancer, tyrosine kinase signaling was characterized in 41 NSCLC cell lines and over 150 NSCLC tumors. See, Rikova, K., Guo, A., et al. Global survey of phosphotyrosine signaling identifies oncogenic kinases in lung cancer. Cell 131:1190-1203 (2007). Profiles of phosphotyrosine signaling were generated and analyzed to identify known oncogenic kinases. Interestingly, ROS kinase was determined to be in the top-ten receptor tyrosine kinases (RTKs) found in both cell lines and tumors. RTKs in this survey were ranked according to phosphorylation rank (phosphorylation level/sample). The results revealed that ROS kinase was highly expressed in one tumor sample and in the NSCLC cell line (HCC78). See, Id. In addition to ROS over-expression in these samples, protein tyrosine phosphatase non-receptor type 11 (PTPN11) and Insulin receptor substrate-2 (IRS-2), earlier reported to be important downstream effectors of ROS in glioblastoma, were found to be highly phosphorylated in ROS-expressing samples. See, Rikova, K., Guo, A., et al. Global survey of phosphotyrosine signaling identifies oncogenic kinases in lung cancer. Cell 131:1190-1203 (2007). Furthermore, several microarray analyses of tumor specimens also revealed significantly elevated ROS-expression levels in 20-30% of patients with NSCLC. See, e.g., Bild, A. H., Yao, G., et al. Oncogenic pathway signatures in human cancers as a guide to targeted therapies. Nature 439:353-357 (2006). Contrasting the results found in brain tumors, elevated ROS expression in lung tumors was observed in both early- and late-stage tumors, suggesting a key role for ROS in the initiation or development rather than progression of lung tumors. See, e.g., Bonner, A. E., Lemon, W. J., et al. Molecular profiling of mouse lung tumors: association with tumor progression, lung development, and human lung adenocarcinomas. Oncogene 23:1166-1176 (2004).

### ROS in Brain Tumors

A number of RTKs are characteristic as markers for nervous system tumors. By way of example, the epidermal growth factor receptor (EGFR) and its associated oncogene Erb-B are noteworthy, as 45-50% malignant gliomas show evidence for EGFR amplification. See, e.g., Yamazaki, H., Fukui, Y., et al. Amplification of the structurally and functionally altered epidermal growth factor receptor gene (c-erbB) in human brain tumors. Mol. Cell Biol. 8:1816-1820 (1988). Other RTKs include: Neu (see, e.g., Bernstein, J. J., Anagnostopoulos, A. V., et al. Human-specific c-neu proto-oncogene protein overexpression in human malignant astrocytomas before and after xenografting. J. Neurosurg. 78:240-251 (1993)), platelet-derived growth factor (PDGF) receptor (see, e.g., Lokker, N. A., Sullivan, C. M., et al. Platelet-derived growth factor (PDGF) autocrine signaling regulates survival and mitogenic pathways in glioblastoma cells. Cancer Res. 62:3729-3735 (2002)), ROS (see, e.g., Jun, H. J., Woolfenden, S., et al. Epigenetic regulation of c-ROS receptor tyrosine kinase expression in malignant gliomas. Cancer Res. 69:2180-2184 (2009)).

In a survey of 45 different human cell lines, ROS was found to be expressed in 56% of glioblastoma-derived cell lines at high levels (i.e., ranging from 10 to 60 transcripts per cell), while not expressed at all or expressed minimally in the remaining cell lines. See, Birchmeier, C., Sharma, S., Wigler, M. Expression and rearrangement of the ROS gene in human glioblastoma cells. Proc. Natl. Acad. Sci. USA 84:9270-9274 (1987). Moreover, no expression of ROS gene was observed in normal, non-neoplastic brain tissues; thus, the high level of ROS expression in glioblastoma seems specific. The failure of ROS detection in lower grade astrocytomas, however, suggests that ROS may play a role in tumor progression rather than initiation. See, Mapstone, T., McMichael, M., Goldthwait, D. Expression of platelet-derived growth factors, transforming growth factors, and the ROS gene in a variety of primary human brain tumors. Neurosurgery 28:216-222 (1991).

### ROS in Stomach, Breast, Liver, Colon, and Kidney Cancers

c-ROS gene was found to be upregulated in gastric cancer induced by oral administration of N-methyl-NO-nitro-N-nitrosoguanidine (MNNG) in rat. See, Yamashita, S., Nomoto, T., et al. Persistence of gene expression changes in stomach mucosae induced by short-term N-methyl-N0-nitro-N-nitrosoguanidine treatment and their presence in stomach cancers. Mutat. Res. 549:185-193 (2004). ROS gene was one of six genes found to be persistently upregulated after 4 weeks from MNNG treatment. ROS gene was found also to be overexpressed (in a number of other genes) in fibroadenoma samples taken from breast tumors of five different patients. It was found to be expressed at levels more than two-fold higher than those in normal tissues. See, e.g., Eom, M., Han, A., et al. ROS expression in fibroadenomas of the breast. Pathol. Int. 58:226-232 (2008). In liver, the induction of hepatic progenitor cells activation in a rat model of liver injury was found to be associated with overexpression of ROS. In addition, overexpression of ROS was also observed in a rat hepatoma cell line. See, e.g., Yovchev, M. I., Grozdanov, P. N., et al. Novel hepatic progenitor cell surface markers in the adult rat liver. Hepatology 45:139-149 (2007). Recently, a global sequencing survey of all tyrosine kinases in 254 cell lines revealed three new ROS mutations in two colon adenocarcinoma and one kidney carcinoma cell lines. See, Ruhe, J. E., Streit, S., et al. Genetic alterations in the tyrosine kinase transcriptome of human cancer cell lines. Cancer Res. 67:11368-11376 (2007).

(iv) Epidermal Growth Factor Receptor (EGFR)

The epidermal growth factor receptor (EGFR) is the cell-surface receptor for members of the epidermal growth factor family (EGF-family) of extracellular protein ligands. See, e.g., Herbst, R. S. Review of epidermal growth factor receptor biology. Int. J. Radiat. Oncol. Biol. Phys. 59:21-26 (2004). EGFR is a member of the ErbB family of receptors, which comprise a subfamily of four (4) closely related receptor tyrosine kinases, which include: ErbB-1 (also known as epidermal growth factor receptor (EGFR), HER1); ErbB-2 (also know as HER 2 in humans and c-neu in rodents); ErbB-3 (also known as HER 3); and ErbB-4 (also known as HER 4). Mutations affecting EGFR expression and/or activity have been shown to be involved in many forms of cancer. EGFR (HER1, erbB1) is expressed or highly expressed in a variety of human tumors including, but not limited to: non-small cell lung cancer (NSCLC), breast, head and neck, gastric, colorectal, esophageal, prostate, bladder, renal, pancreatic, and ovarian cancers.

ErbB receptors (170 kDa) are comprised of an extracellular region or ectodomain that contains approximately 620 amino acid residues, a single transmembrane-spanning region, and a cytoplasmic tyrosine kinase domain. The extracellular region of each ErbB family member is made up of four subdomains: L1, CR1, L2, and CR2—wherein “L” denotes a leucine-rich repeat domain and “CR” a cysteine-rich region. These subdomains are also referred to as domains I-IV, respectively. See, e.g., Ward, C. W., Lawrence, M. C., et al. The insulin and EGF receptor structures: new insights into ligand-induced receptor activation. Trends Biochem. Sci. 32:129-137 (2007).

EGFR exists on the cell surface and is activated by binding of its specific ligands, including epidermal growth factor and transforming growth factor α (TGFα). As previously discussed, ErbB2 has no known direct activating ligand, and may be in an activated state constitutively or become active upon heterodimerization with other ErbB family members. Upon activation by its growth factor ligands, EGFR undergoes a transition from an inactive monomeric form to an active homodimer. However, there is also some evidence that preformed inactive dimers may also exist before growth factor ligand binding. In addition to forming homodimers, EGFR may pair with another member of the ErbB receptor family (e.g., ErbB2/Her2/neu) to create an activated heterodimer. There is also evidence to suggest that clusters of activated EGFRs form, although it remains unclear whether this clustering is important for activation itself or occurs subsequent to activation of individual dimers.

EGFR dimerization stimulates its intrinsic intracellular protein/tyrosine kinase activity. As a result, autophosphorylation of several tyrosine amino acid residues in the carboxyl-terminal domain of EGFR occurs. These include Tyr992, Tyr1045, Tyr1068, Tyr1148, and Tyr1173. See, e.g., Downward, J., Parker, P., Waterfield, M. D. Autophosphorylation sites on the epidermal growth factor receptor. Nature 311:483-485 (1984). This autophosphorylation elicits downstream activation and signaling by several other proteins that associate with the phosphorylated tyrosines through their own phosphotyrosine-binding SH2 domains. These downstream signaling proteins initiate several signal transduction cascades (principally the MAPK, Akt, and JNK pathways), leading to DNA synthesis and cell proliferation. See, e.g., Oda, K., Matsuoka, Y., et al. A comprehensive pathway map of epidermal growth factor receptor signaling. Mol. Syst. Biol. 1:205-210 (2005). Such proteins modulate phenotypes, including but not limited to: cell migration, cell adhesion, and cell proliferation. In addition, activation of the receptor is important for the innate immune response in human skin. See, e.g., Roupé, K. M.; Nybo, M., et al. Injury is a major inducer of epidermal innate immune responses during wound healing. J. Investigative Dermatol. 130:1167-1177 (2010). The kinase domain of EGFR can also cross-phosphorylate tyrosine residues of other receptors it is aggregated with and can itself, be activated in that same manner. See, e.g., Oda, K., Matsuoka, Y., et al. A comprehensive pathway map of epidermal growth factor receptor signaling. Mol. Syst. Biol. 1:205-210 (2005).

The importance of EGF-EGFR in protein phosphorylation and in tumorigenesis, and subsequently the EGF-EGFR signaling axis has taken an important role in developmental biology and cancer research. Activated EGFR recruits a number of downstream signaling molecules, leading to the activation of several major pathways that are important for tumor growth, progression, and survival. See, e.g., Lo, H. W., Hung, M. C. Nuclear EGFR signaling network in cancers linking EGFR pathway to cell cycle progression, nitric oxide pathway and patient survival. Br. J. Cancer 94:184-188 (2006). The main pathways downstream of EGFR activation include those mediated by PLC-γ-PKC, Ras-Raf-MEK, PI-3K-Akt-mTOR, and JAK2-STAT3. Similar to EGFR, the EGFRvIII variant is primarily localized on the cell-surface where it activates several signaling modules. However, unlike EGFR, EGFRvIII is constitutively active independent of ligand stimulation, in part, due to its loss of a portion of the ligand-binding domain.

While EGFR over-expression is found in many types of human cancers, EGFRvIII is predominantly detected in malignant gliomas. Both EGFR and EGFRvIII play critical roles in tumorigenesis and in supporting the malignant phenotypes in human cancers. Consequently, both receptors are targets of anti-cancer therapy. Several EGFR-targeting small molecule kinase inhibitors and therapeutic antibodies have been approved by the FDA to treat patients with breast cancer, colorectal cancer, non-small cell lung cancer (NSCLC), squamous cell carcinoma of the head and neck, and pancreatic cancer. Despite the extensive efforts invested in the preclinical and clinical development of EGFR-targeted therapy, the currently utilized treatments have demonstrated only modest effects on most cancer types, with the exception of NSCLC that expresses gain-of-function EGFR mutants. However, almost all of these aforementioned NSCLC patients eventually developed resistance to small molecule EGFR kinase inhibitors. See, e.g., Bonanno, L., Jirillo, A., Favaretto, A. Mechanisms of acquired resistance to epidermal growth factor receptor tyrosine kinase inhibitors and new therapeutic perspectives in non small cell lung cancer. Curr. Drug Targets 12:922-933 (2011). This acquired resistance has been shown to be linked to a secondary EGFR T790M mutation in approximately half of patients. This resistance can be attributed to other potential mechanisms, such as, uncontrolled activation of MET (see, e.g., Engelman, J. A., Janne, P. A. Mechanisms of acquired resistance to epidermal growth factor receptor tyrosine kinase inhibitors in non-small cell lung cancer. Clin. Cancer Res. 14:2895-2899 (2008)) and subsequent MET-mediated HER3 activity (see, e.g., Arteaga, C. L. HER3 and mutant EGFR meet MET. Nat. Med. 13:675-677 (2007)) and activated insulin-like growth factor-1 receptor (see, e.g., Morgillo, F., Kim, W. Y., et al. Implication of the insulin-like growth factor-IR pathway in the resistance of non-small cell lung cancer cells to treatment with gefitinib. Clin. Cancer Res. 13:2795-2803 (2007)). As lung cancer-associated EGFR mutations are either absent or very rare in other tumor types, there is an important need to identify the mechanisms underlying tumor resistance to anti-EGFR agents in order to derive sensitization strategies that can be used to overcome this resistance.

(v) Insulin-Like Growth Factor 1 Receptor Kinase

The Insulin Growth Factor 1 Receptor (IGF1R) kinase is a member of the IGF axis, a family of insulin receptor related and insulin growth factor related proteins that are important in endocrine function and cancer. See, e.g., Arnaldez and Helman, Targeting the insulin growth factor receptor 1. Hematol. Oncol. Clin. North. Am. 26(3):527-542 (2012). IGF1R has a high degree of structural similarity to the insulin receptor and modulates cell growth and proliferation through several key proteins including PI3K, IRS, MAPK, JAK/STAT, and others. See, FIG. 44; see, e.g., Fidler, et al, Targeting the insulin-like growth factor receptor pathway in lung cancer: problems and pitfalls. Ther. Adv. Med. Oncol. 4(2):51-60 (2012). IGF1R is important in a variety of cancers including, but not limited to, lung, colon, breast, sarcoma and prostate cancer. See, e.g., Gombos, et al, Clinical Development of Insulin-Like Growth Factor Receptor-1 (IGF1R) Inhibitors: At the Crossroad. Invest. New Drugs 30(6):2433-2442 (2012); Gallagher and LeRoith, IGF, Insulin and Cancer. Endocrinology 152(7):2546-2451 (2011).

Like many receptor tyrosine kinases, IGF1R homodimerizes at the cell membrane and transduces signals through the various signaling pathways. Additionally IGF1R can form heterodimers with other receptors including, but not limited to, the insulin receptor and EGFR2 (HER-2). The heterodimerization with EGFR2 has been proposed to contribute to Trastuzumab resistance in vitro and may have important in vivo implications as well. See, e.g., Maki, Insulin-like Growth Factors and Their Role in Growth, Development, and Cancer. J. Clin. Oncol. 28(33):4985-4995 (2011). IGF1R is the subject of many laboratory studies and more than 60 clinical trials have been initiated to evaluate agents that putatively target IGF1R. See, e.g., Gombos, et al, Clinical Development of Insulin-Like Growth Factor Receptor-1 (IGF1R) Inhibitors: At the Crossroad. Invest. New Drugs 30(6):2433-2442 (2012). However, no compound has yet been approved by the FDA that specifically modulates IGF1R function. Heidegger and co-workers have suggested that this may be due to the complex and essential role IGF1R has in normal physiology. See, e.g., Heidegger, et al., Targeting the insulin-like growth factor network in cancer therapy. Cancer Biol. Ther. 11(8):701-707 (2011).

B. DNA Repair Enzymes

(i) ERCC1-XPF DNA Repair Endonuclease

DNA excision repair protein ERCC-1 is a protein that in humans is encoded by the ERCC1 gene. The function of the ERCC1 protein is predominantly in nucleotide excision repair (NER) of damaged DNA. NER is one of five separate DNA repair mechanisms that also include: recombination repair, base excision repair, mismatch repair, and translesion synthesis. Nucleotide excision repair (NER) in eukaryotes is initiated by either Global Genome NER (GG-NER) or Transcription Coupled NER (TC-NER) which involve distinct protein complexes, each recognizing damaged DNA. Thereafter, subsequent steps in GG-NER and TC-NER share a final common excision and repair pathway which include the following steps: (i) transcription factor II H (TFIIH) separates the abnormal strand from the normal strand; (ii) xeroderma pigmentosum group G (XPG) cuts 3′ to the damaged DNA: (iii) replication protein A (RPA) protects the “normal”, non-damaged strand; (iv) xeroderma pigmentosum group A (XPA) isolates the damaged segment on the strand to be cut; and (v) ERCC1 and xeroderma pigmentosum group F (XPF) cut 5′ to the damaged DNA. ERCC1 appears to have a crucial role in stabilizing and enhancing the functionality of the XPF endonuclease. The excised single-stranded DNA (approximately 30 nucleotides in length) and the attached NER proteins are excised and removed. DNA polymerases and ligases then fill in the gap left by the excision of the damaged DNA strand using the normal strand as a template.

In mammals, the ERCC1-XPF protein complex also removes non-homologous 3′ tail ends in homologous recombination. The ERCC1-XPF complex is a structure-specific endonuclease involved in the repair of damaged DNA. ERCC1-XPF performs a critical incision step in nucleotide excision repair (NER), and is also involved in the repair of DNA interstrand crosslinks (ICLs) and some double-strand breaks (DSBs). See, e.g., Ahmad, A., Robinson, A., et al. ERCC1-XPF endonuclease facilitates DNA double-strand break repair. Mol. Cell. Biol. 28:5082-5092 (2008). A fraction of ERCC1-XPF is localized at telomeres, where it is implicated in the recombination of telomeric sequences and loss of telomeric overhangs at deprotected chromosome ends. In telomere maintenance, ERCC1-XPF degrades 3′ G-rich overhangs (see, e.g., Kirschner, K., Melton, D. W. Multiple roles of the ERCC1-XPF endonuclease in DNA repair and resistance to anticancer drugs. Anticancer Res. 30:3223-2332 (2010)) and various other related functions (see, e.g., Rahn, J. J., Adair, G. M., Nairn, R. S. Multiple roles of ERCC1-XPF in mammalian interstrand crosslink repair. Environ. Mol. Mutagen. 51:567-581 (2010)).

Deficiency of either ERCC1 or XPF in humans results in a variety of conditions, which include the skin cancer-prone disease xeroderma pigmentosum (XP), a progeroid syndrome of accelerated aging, or cerebro-oculo-facioskeletal syndrome (COFS). These diseases are extremely rare in the general population and therefore mice with low levels of either ERCC1 or XPF have been generated and studied extensively. These murine models clearly illustrate the importance of DNA repair in preventing aging-related tissue degeneration.

(ii) Ribonucleotide Reductase

Ribonucleotide reductase (RNR) is a multimeric protein that reduces the 2′ hydroxyl on ribonucleotides to a 2′ hydrogen yielding deoxyribonucleotides that can be utilized in DNA synthesis and DNA repair. See, e.g., Hofer, et al., DNA building blocks: Keeping control of manufacture. Crit. Rev. Biochem. Mol. Biol. 47:50-63 (2012). Human RNR is composed of the subunits M1 (α) and M2 (β or β′) that associate into multimeric forms including a heterodimeric tetramer (α₂β₂) and other complex multimers (αₙ(β₂)ₘ; wherein n=2, 4, or 6 and m=1 or 3. See, e.g., Wang, et al, Mechanism of inactivation of human ribonucleotide reductase with p53R2 by gemcitabine 5′-disphosphate. Biochemistry 48(49):11612-11621 (2009). The M1 subunit (a subunit; larger subunit) of RNR binds the ribonucleotide substrate and is catalytic while the M2 subunit ((3 subunit; smaller subunit) contains the diferric tyrosyl radical that is required for catalysis. See, e.g., Wan, et al., Enhanced subunit interactions with gemcitabine-5′-diphosphate inhibit ribonucleotide reductases. Proc. Natl. Acad. Sci. U.S.A. 104(36):14324-14329 (2010); Morandi, Biological agents and gemcitabine in the treatment of breast cancer. Annals Oncol. 17:180-186 (2006); Fairman, et al., Structural basis for allosteric regulation of human ribonucleotide reductase by nucleotide-induced oligomerization. Nat. Struct. Mol. Biol. 18(3):316-322 (2011). RNR is required for de novo DNA synthesis and DNA repair and is, therefore, critical for cell growth and proliferation. See, e.g., Wang, et al, Mechanism of inactivation of human ribonucleotide reductase with p53R2 by gemcitabine 5′-disphosphate. Biochemistry 48(49):11612-11621 (2009).

Unfortunately, only a few drugs have been developed to target human RNR. See, e.g., Wijerathna, et al., Targeting the large subunit of human ribonucleotide reductase for cancer chemotherapy. Pharmaceuticals 4(10):1328-1354 (2010). Gemcitabine is a recently developed small molecule that targets RNR (specifically, Gemcitabine diphosphate targets RNR) and has been used as a single agent and in combination with other agents to treat a range of cancers including non-small cell lung cancer (NSCLC), pancreatic cancer, ovarian cancer and other tumor types. See, e.g., Favaretto, Non-platinum combination of gemcitabine in NSCLC. Annals Oncol. 17:v82-v85 (2006); Long, et al., Overcoming Drug Resistance in Pancreatic Cancer. Expert Opin. Ther. Targets 15(7):817-828 (2011); Matsuo, et al., Overcoming Platinum Resistance in Ovarian Carcinoma. Expert Opin. Investig. Drugs 19(100):1339-1354 (2010). Hydroxyurea is a classical agent targeting RNR and has been used in combination with radiation to treat head and neck cancer and cervical cancer. See, e.g., Chapman and Kinsella, Ribonucleotide reductase inhibitors: A new look at an old target for radiosensitization. Frontiers Oncol. 1:1-6 (2009). RNR has been found to be elevated in some NSCLC patients and development of agents that target and modulate RNR function would be useful in the clinic. See, e.g., Ren, et al., Individualized chemotherapy in advanced NSCLC patients based on mRNA levels of BRCA1 and RRM1. Chin. J. Cancer Res. 24(3):226-231 (2012); Ceppi, et al., ERCC1 and RRM1 gene expressions but not EGFR are predictive of shorter survival in advanced non-small-cell lung cancer treated with cisplatin and gemcitabine. Ann. Oncol. 17(12):1818-1825 (2006); Souglakos, et al., Ribonucleotide reductase subunits M1 and M2 mRNA expression levels and clinical outcome of lung adenocarcinoma patients treated with docetaxel/gemcitabine. Br. J. Cancer 98:1710-1715 (2008).

C. Structural Proteins

(i) Tubulin

The structural proteins that comprise the microtubule arrays in vivo are critical for cell division, cell proliferation and a range of other intracellular processes. See, e.g., Harrison, et al., Beyond taxanes: A review of novel agents that target mitotic tubulin and microtubules, kinases, and kinesins. Clin. Adv. Hematol. Oncol. 7:54-64, (2009).

Microtubules consist primarily of α and β tubulin subunits but also contain numerous other microtubule proteins. Oncology drugs that target tubulin have been developed and include drugs in the taxane, epothilone, and vinca alkaloid families. See, e.g., Gascoigne and Taylor, How do anti-mitotic drugs kill cancer cells. J. Cell. Sci. 122:2579-2585 (2009). Agents with the ability to stabilize the tubulin protein within microtubules can result in mitotic arrest and eventually cell death (apoptosis). However, many of the drugs that target tubulin protein and microtubules have side-effects that can be dose-limiting or necessitate the withdrawal of treatment. For example, paclitaxel, a well-known and highly utilized anti-cancer agent exerts its effect primarily by stabilizing tubulin (see, e.g., Xiao, et al., Insights into the mechanism of microtubule stabilization by Taxol, Proc. Natl. Acad. Sci, U.S.A. 103(27):10166-10173 (2006)), but neurotoxicity, manifested primarily as peripheral neuropathy, is a common side effect of taxane-based chemotherapy.

Mechanisms behind chemotherapy-induced peripheral neuropathy (CIPN) are complex, involve damage to the peripheral nerve, and include axonopathy, myelinopathy, and neuronopathy. See, e.g., Lee and Swain, Peripheral neuropathy induced by microtubule-stabilizing agents. J. Clin. Oncol. 24:1633-1642 (2006). Amifostine, glutathione, glutamine/glutamate, calcium/magnesium infusions, neurotrophic factors, NGF, gabapentin, vitamin E, N-acetylcysteine, diethyldithiocarbamate, erythropoietin, and carbamazepine are among the many agents that have been evaluated for use as potential neuroprotective agents. See, e.g., Cavaletti, et al., Neurotoxic effects of antineoplastic drugs: The lesson of pre-clinical studies. Front. Biosci. 13:3506-3524 (2008). However, despite promising results in some clinical trials, no approved therapy has yet proven effective for the prevention or mitigation of chemotherapy-induced peripheral neuropathy (CIPN), and none of the therapies that have been evaluated thus far have become a standard of care, or have otherwise provided definitive evidence of benefit in the prevention, mitigation, or treatment of CIPN. See, e.g., Parker, et al., BNP7787-mediated modulation of paclitaxel- and cisplatin-induced aberrant microtubule protein polymerization in vitro. Mol. Cancer Ther. 9(9):2558-2567 (2010). Additionally, many of these therapies have adverse side-effects which may limit their utility in patients, and it is presently unknown if there is significant concurrent potential interference with the anti-tumor activity of chemotherapy.

D. Prenyltransferases

Human protein prenyltransferases include the proteins farnesyltransferase (FTase), geranylgeranyltransferase I (GGTase I), and geranylgeranyltransferase II (GGTase II). These prenyltransferases transfer lipophilic isoprene groups that enable the prenylated substrates to more avidly associate with cellular membranes. The proteins that are prenylated by the human protein prenyltransferases are involved in a range of intracellular pathways and processes important for cell growth and proliferation. See, e.g., Holstein and Hohl, Is there a future for prenyltransferases inhibitors in cancer therapy? Curr. Opin. Pharmacol. 12:704-709 (2012); Maurer-Stroh, et al., Protein prenyltransferases. Genome Biol. 4:212-221 (2003). Although cancer treating agents that specifically target prenyltransferases have not yet received FDA approval in the United States, prenyltransferases represent attractive targets for drug discovery especially within the area of oncology, as further discussed below. See, e.g., Holstein and Hohl, Is there a future for prenyltransferases inhibitors in cancer therapy? Curr. Opin. Pharmacol. 12:704-709 (2012). Targeting prenyltransferases requires a global cellular perspective. For example, inhibition of the prenyltransferases, FTase and GGTase I alone, might not be an effective anti-cancer approach were it not for the fact that the substrates that are post-translationally modified by these prenyltransferases are essential in regulating many different cell growth and cell survival signaling pathways. A specific example of FTase- and GGTase-mediated prenylation that is important and required for the regulation of cell proliferation and cell survival involves the RAS protein family.

By way of non-limiting example, RAS proteins include: KRAS, HRAS, and NRAS. RAS proteins have high sequence similarity/identity and regulate proteins that have important roles in cell proliferation-related pathways, including but not limited to, MAPK, STAT, Raf, MEK, and ERK; as well as proteins that are key in anti-apoptotic pathways, including but not limited to, PI3K and Akt. See, e.g., Vadakara and Borghael, Personalized medicine and treatment approaches in non-small-cell lung carcinoma. Pharmacogenomics Personalized Med. 5:113-123 (2012); Riely, et al., KRAS mutations in non-small cell lung cancer. Proc. Am. Thorac. Soc. 6:201-205 (2009). RAS protein mutations and/or functional dysregulation has been implicated in up to one-third of all human cancers. See, e.g., Baines, et al., Inhibition of Ras for cancer treatment: the search continues. Future Med. Chem. 3:(14) 1787-1808 (2011); Santarpia, et al., Targeting the mitogen-activated protein kinase RAS-RAF signaling pathway in cancer therapy. Expert Opin. Ther. Targets 16(1):113-119 (2012). For example, KRAS is an important oncology target that is commonly mutated in 80% of pancreatic cancer patients, 20% of all non-small cell lung cancer (NSCLC) patients, and is also often mutated in colorectal cancer patients as well. See, e.g., Adjei, Blocking onocogenic Ras signaling for cancer therapy. J. Natl. Cancer Inst. 93:(14) 1062-1074 (2001); Johnson and Heymach, Farnesyl transferase inhibitors for patients with lung cancer. Clin. Cancer. Res. 10:4254s-4257s (2004); Baines, et al., Inhibition of Ras for cancer treatment: the search continues. Future Med. Chem. 3(14):1787-1808 (2011). RAS proteins are substrates for prenyltransferases and, regardless of their mutational state, must be prenylated to be able to translocate to the cell membrane and transduce signals that regulate cell proliferation and apoptosis. See, e.g., Sebti, Blocked pathways: FTIs shut down oncogene signals. The Oncologist 8(Suppl 3):30-38 (2003). As a consequence of these important activities, proteins that prenylate RAS, such as farnesyltransferase (FTase) and geranylgeranyltransferase (GGTase), are attractive targets for anti-cancer drug development efforts.

Members of the RAS protein family are substrates for both FTase and GGTase I and effective inhibitors of RAS, which work by inhibiting prenylation and, therefore, localization to the membrane, must inhibit both FTase and GGTase I. Given the fact that RAS proteins are important in NSCLC (see, e.g., Vadakara and Borghael, Personalized medicine and treatment approaches in non-small-cell lung carcinoma. Pharamcogen. Personalized Med. 5:113-123 (2012); Riely, et al., KRAS mutations in non-small cell lung cancer. Proc. Am. Thorac. Soc. 6:201-205 (2009); Johnson and Heymach, Farnesyl transferase inhibitors for patients with lung cancer. Clin. Cancer Res. 10:4254s-4257s (2004)) as well as in pancreatic, colorectal, and other cancers (see,e.g., Baines, et al., Inhibition of Ras for cancer treatment: the search continues. Future Med. Chem. 3(14):1787-1808 (2011)), the development of compounds that modulate the function of prenyltransferases like FTase, which in turn modulate the function of both wild type and mutated RAS proteins, are clearly important.

(i) Farnesyltransferase

Farnesyltransferase (FTase) catalyzes the addition of a 15 carbon moiety onto key proteins, including but not limited to: (i) the RAS family of proteins; (ii) kinetochore proteins; (iii) cGMP phosphodiesterase; (iv) peroxisomal proteins; (v) nuclear lamina proteins; (vi) heat shock homologs; (vii) rhodopsin kinase; and similar proteins. See, e.g., Maurer-Stroh, et al., Protein prenyltransferases. Genome Biol. 4:212-221 (2003). A key target of FTase is the RAS protein family (e.g., HRAS, KRAS and NRAS). RAS modulates a wide range of intracellular signaling pathways the regulate cell growth, cell proliferation, and apoptosis. See, FIG. 78; Appels, et al., Development of Farnesyl Transferase Inhibitors: A Review. 10:565-578 (2005).

E. Oxidoreductases (Redox Enzymes)

Oxidoreductases are enzymes that catalyzes the transfer of electrons from one molecule (i.e., the reductant, also called the hydrogen or electron donor) to another (i.e., the oxidant, also called the hydrogen or electron acceptor). This group of enzymes usually utilizes NADPH or NAD⁺ as cofactors.

(i) Peroxiredoxin (Prx)

Peroxiredoxins (Prxs) are a ubiquitous family of small (22-27 kDa) non-seleno peroxidases that functions as anti-oxidants and also control cytokine-induced peroxide levels and thereby mediate signal transduction in mammalian cells. Unlike Trx possessing the active double-cysteine region and forming the intramolecular disulfide bond when oxidized, Prx have no such regions; however, the easily oxidized Cys residues present in their structure can form intermolecular disulfide bonds. There are six mammalian isoforms that have been currently identified. See, e.g., Rhee, S., Chae, H., Kim, K. Peroxiredoxins: a historical overview and speculative preview of novel mechanisms and emerging concepts in cell signaling. Free Radical Biol. Med. 38:1543-1552 (2005). Although their individual roles in cellular redox regulation and antioxidant protection are quite distinct, they all catalyze peroxide reduction of H₂O₂, organic hydroperoxides, and peroxynitrite. They are found to be expressed ubiquitously and in high levels, suggesting that they are both an ancient and important enzyme family.

Mammalian cells express six Prx isoforms (Prx 1-6), which can be divided into three subgroups as follow: (i) 2-Cys Prx proteins, which contain both the N- and C-terminal-conserved Cys residues and require both of them for catalytic function; (ii) atypical 2-Cys proteins, which contain only the N-terminal Cys but require one additional, nonconserved Cys residue for catalytic activity; and (iii) 1-Cys Prx proteins, which contain only the N-terminal Cys and require only the conserved one for catalytic function. Four (Prx 1-4) of the six mammalian Prxs belong to the 2-Cys subgroup and have the conserved N- and C-terminal Cys residues that are separated by 121 amino acid residues. Both Prx 1 (NKEF A, PAG, MSP23, OSF3, HBP23) and Prx 2 (NKEF B, Calpromotin, Torin) proteins consist of 199 amino acid residues and exist in cytosol (various alternative names given without reference to peroxidase function are in parentheses). The 257-amino acid sequence of Prx 3 (MER5, SP22) deduced from the cDNA sequence of MER5 is substantially larger than the 195 amino acid residue sequence of SP22, as determined directly by peptide sequencing of SP22 purified from mitochondria of bovine adrenal cortex. The additional 62 residues at the N-terminus were proved to be the mitochondrial-targeting sequence. Prx 4 (AOE372, TRANK) was identified as a protein that interacts with Prx I by the yeast two-hybrid assay. See, e.g., Jin, D. Y.; Chae, H. Z.; et al. Regulatory role for a novel human thioredoxin peroxidase in NF-kappaB activation. J. Biol. Chem. 272:30952-30961 (1997). This protein-protein interaction is probably because a small portion of Prx proteins forms heterodimers. Prx 4 contains the N-terminal signal sequence for secretory proteins and found in culture medium. As demonstrated first with yeast TPx, the N-terminal Cys is oxidized by peroxides to cysteine sulfenic acid, which then reacts with the C-terminal-conserved cysteine of the other subunit to form an intermolecular disulfide. The reduction of the intermolecular disulfide is specific to thioredoxin (Trx) and could not be achieved by glutathione (GSH) or glutaredoxin. Thus, mutant 2-Cys Prx proteins that lack either the N-terminal or C-terminal Cys residues do not exhibit Trx-coupled peroxidase activity. Mammalian cells contain mitochondria-specific Trx and TrxR, suggesting that Prx 3 together with the mitochondria-specific Trx and TrxR provide a primary line of defense against H₂O₂ produced by the mitochondrial respiratory chain. See, e.g., Rhee, S., Chae, H., Kim, K. Peroxiredoxins: a historical overview and speculative preview of novel mechanisms and emerging concepts in cell signaling. Free Radical Biol. Med. 38:1543-1552 (2005).

The amino acid sequence identity among the four mammalian 2-Cys (Prx 1 to Prx 4) enzymes is 70%, with the homology being especially marked in the regions surrounding the conserved N- and C-terminal Cys residues. The atypical 2-Cys Prx, Prx5, was identified as the result of a human EST database search with the N-terminal-conserved sequence (KGKYVVLFFYPLDFTFVCP) of the 2-Cys Prx enzymes. The 162-amino acid Prx 5 shares only ˜10% sequence identity with the four mammalian 2-Cys Prx proteins and the sequence surrounding the conserved NH2-terminal Cys (Cys⁴⁷) (KGKKGVLFGVPGAFTPGCS) is only 52% identical to the search sequence. The C-terminal region of PrxV is smaller than those of 2-Cys Prx enzymes and lacks the conserved sequence containing the C-terminal Cys of the latter enzymes. Both human and mouse Prx 5 sequences contain Cys residues at positions 72 and 151, in addition to the conserved Cys⁴⁷. However, the sequences surrounding Cys⁷² and Cys¹⁵¹ are not homologous to those surrounding the C-terminal conserved Cys residue of 2-Cys Prx enzymes, and the distances between Cys₄₇ and these other two Cys residues are substantially smaller than the 121 amino acid residues that separate the two conserved Cys residues in typical 2-Cys Prx enzymes. Cys⁴⁷ is the site of oxidation by peroxides, and the resulting oxidized Cys⁴⁷ reacts with the sulfhydryl group of Cys¹⁵¹ to form a disulfide linkage, which was initially suggested to be intramolecular based on biochemical data. However, recent crystal structures indicate that oxidation of Prx 5 first gives rise to two intermolecular disulfide bonds, which might then rearrange to form intramolecular disulfides. See, e.g., Evrard, C.; Capron, A. et al. Crystal structure of a dimeric oxidized form of human peroxiredoxin 5. J. Mol. Biol. 337:1079-1090 (2004). This is possible because the two disulfide bonds of the oxidized dimer are very close to one another. The disulfide formed by Prx 5 is reduced by thioredoxin, but not by glutaredoxin or glutathione. Although only the N-terminal Cys residue is conserved in Prx 5, it is designated as 2-Cys Prx enzyme because its function is dependent on two Cys residues. Prx 5 is localized intracellularly to cytosol, mitochondria, and peroxisomes.

The full-length cDNA (ORF06) for a human 1-Cys Prx, also termed Prx 6, was identified without any reference to peroxidase activity as the result of a sequencing project with human myeloid cell cDNA. Upon exposure to H₂O₂, the N-terminal Cys-SH of Prx 6, which corresponds to Cys⁴⁷ of human Prx 6, is readily oxidized. However, the resulting Cys-SOH does not form a disulfide because of the unavailability of another Cys-SH nearby. In addition to the Cys⁴⁷ of human Prx 6, some 1-Cys Prx members contain other Cys residues, such as Cys⁹¹ of the human enzyme. However, neither Cys⁹¹ itself nor the sequence surrounding this residue is conserved among the 1-Cys Prx members. The Cys-SOH of oxidized 1-Cys Prx can be reduced by non-physiological thiols such as DTT. The identity of its redox partner is not yet clear. GSH has been suggested to be the physiological donor for 1-Cys Prx. However, several laboratories have failed to detect GSH-supported peroxidase activity of 1-Cys-Prx. Prx 6 is a cytosolic enzyme.

Although the catalytic activity of Prx towards H₂O₂ (10⁵-10⁶/M/sec) is lower than that of glutathione peroxidase (10⁸/M/sec) and catalase (10⁶/M/sec), they play an important role in detoxification of H₂O₂. Reduction of H₂O₂ by all Prx isoforms passes through formation of sulfenic acid (Cys-SOH) due to oxidation of SH-group of the Cys residue; however, the mechanism of the peroxidase reaction slightly differs in the different Prx isoforms. Since H₂O₂ can rapidly transform into highly toxic reactive oxygen species (ROS), such as O₂⁻ radicals, elevation of the levels of ROS can lead to development of oxidative stress causing deleterious physiological effects, including but not limited to: (i) DNA breakage; (ii) linkages in protein molecules; and (iii) activation of lipid peroxidation. A physiological role of Prx associated with enzymatic degradation of H₂O₂ is particularly significant in erythrocytes, in which these enzymes are ranked second or third place in overall cellular protein content.

An important role of Prx in defense against oxidative stress was demonstrated in a series of studies with knockout of genes corresponding to Prx. Hemolytic anemia, characterized by hemoglobin instability developed, in PRDX1 gene knockout mice. See, e.g., Neumann, C. A., Krause, D. S., et al. Nature 424:561-565 (2003). In PRDX2 gene knockout mice, a significant decrease of lifespan was also accompanied by development of anemia. In both cases, the knockout of the corresponding gene caused a significant elevation of ROS in erythrocytes. The PRDX6 gene knockout mice were characterized by low survival, high level of protein oxidation, and significant injury of kidneys, liver, and lungs. It should be noted that in this case the expression of antioxidant enzymes, such as catalase, glutathione peroxidase, and Mn-SOD did not differ from that in wild-type mice. The results of these studies suggest that function of Prx 6 cannot be compensated by expression of other genes. See, e.g., Wang, X., Phelan, S. A., et al. J. Biol. Chem. 278:25179-25190 (2003).

The expression of genes encoding different Prx isoforms has cellular, tissue, and organ specificity. Prx 1 is the most widely represented and highly expressed member of the peroxiredoxin family in virtually all organs and tissues of mice and humans, both in normal tissues and malignant tumors. See, e.g., Li, B., Ishii, T., et al. J. Biol. Chem. 277:12418-12422 (2002). In particular, it should be noted that the PRDX1 gene is widely expressed in various areas of the central and peripheral nervous system with expression specificity depending on the cell type. High expression of the PRDX4 gene is characteristic of liver, testes, ovaries, and muscles, whereas low expression is observed in small intestine, placenta, lung, kidney, spleen, and thymus.

It is well known that the production of reactive oxygen species (ROS), such as O₂⁻ radicals and cellular redox state play an important role in regulation of the cell cycle and cell proliferation (see, e.g., Sauer, H., Wartenberg, M., Hescheler, J. Cell. Physiol. Biochem. 11:173-186 (2001)) and that antioxidant enzymes, such as glutathione peroxidase and Mn-SOD, are also involved in cell cycle regulation with an increase in ROS production causing an acceleration the cell cycle in fibroblast culture. See, e.g., Oberley, T. D. Am. J. Pathol. 160:403-408 (2002). Similarly, it was also shown in embryonic murine fibroblasts that the cellular level of ROS correlates with the cell cycle time; wherein overexpression of the SOD2 gene inhibits cell proliferation. See, e.g., Li, N., Oberley, T. D. J. Cell. Physiol. 177:148-160 (1998).

The association of Prx 1 with cell proliferation dates from early studies. In particular, it was shown that expression of the PRDX1 gene was appreciably higher in Ras-transfected epithelial cells compared with the wild-type cells. See, e.g., Prosperi, M. T., Ferbus, D., et al. J. Biol. Chem. 268:11050-11056 (1993). Moreover, it was found that Prx 1 interacts with c-Abl and c-Myc protein kinases playing an important role in regulation of cell proliferation. See, e.g., Wen, S.-T., VanEtten, R. A. Genes Dev. 11:2456-2467 (1997). Prx 1 has also been shown to be capable of regulating the tyrosine kinase activity of c-Abl (by binding with its third structural domain), which leads to restriction of the transforming ability of c-Abl. See, Id. Accordingly, it has been hypothesized that the reversible binding of Prx 1 with c-Abl can serve as a key cell cycle regulator. Prx 1 is also capable of binding with c-Myc via the c-Myc-transactivating domain (see, e.g., Mu, Z. M., Yin, X. Y., Prochownik, E. V. J. Biol. Chem. 277:43175-43184 (2002)), with a decrease in expression of a series of genes specific for activity of c-Myc being observed in the case of over-expression of the PRDX1 gene. Progression of malignant tumors such as lymphomas, sarcomas, and carcinomas is observed in PRDX1 knockout mice. See, e.g., Neumann, C. A., Krause, D. S., et al. Nature 424:561-565 (2003). Accordingly, Prxs are thought to play a role in tumor suppression. Based upon the aforementioned data, one can conclude that the elevation of peroxiredoxin expression inhibits apoptosis, enhances antioxidant effect, and regulates cell proliferation.

(ii) Glutathione and Glutaredoxin System

Glutathione (GSH) is the predominant nonprotein thiol in cells where it plays essential roles as an enzyme substrate and a protecting agent against xenobiotic compounds and oxidants. See, e.g., Dickinson, D. A., Forman, H. J. Cellular glutathione and thiol metabolism. Biochem. Pharmacol. 64:1019-1026 (2002). Glutathione, maintained in the reduced state by glutathione reductase, is able to transfer its reducing equivalents to several enzymes, such as glutathione peroxidases (GPx), glutathione transferases (GSTs), and glutaredoxins. The latter, similar to thioredoxin, can interact with ribonucleotide reductase and with several other proteins involved in cellular signaling and transcription control, such as NF-κB, PTP-1B, PKA, PKC, Akt, and ASK1. See, e.g., Lu, J., Chew, E. H., Holmgren, A. Targeting thioredoxin reducatse is a basis for cancer therapy. Proc. Natl. Acad. Sci. USA 104:12288-12293 (2007). Mammalian cells contain a cytosolic (Grx1) and a mitochondrial (Grx2) glutaredoxin. Mitochondria contain a second glutaredoxin (Grx5), which is homologous to yeast Grx5 in bearing a single cysteine residue at its active site.

(a) Glutathione

Glutathione (GSH), a tripeptide (α-glutamyl-cysteinyl-glycine) serves a highly important role in both intracellular and extracellular redox balance. It is the main derivative of cysteine, and the most abundant intracellular non-protein thiol, with an intracellular concentration approximately 10-times higher than other intracellular thiols. Within the intracellular environment, glutathione (GSH) is maintained in the reduced form by the action of glutathione reductase and NADPH. Under conditions of oxidative stress, however, the concentration of GSH becomes markedly depleted. Glutathione functions in many diverse roles including, but not limited to, regulating antioxidant defenses, detoxification of drugs and xenobiotics, and in the redox regulation of signal transduction. As an antioxidant, glutathione may serve to scavenge intracellular free radicals directly, or act as a co-factor for various other protection enzymes. In addition, glutathione may also have roles in the regulation of immune response, control of cellular proliferation, and prostaglandin metabolism. Glutathione is also particularly relevant to oncology treatment because of its recognized roles in tumor-mediated drug resistance to cancer treating agents and ionizing radiation. Glutathione is able to conjugate electrophilic drugs such as alkylating agents and cisplatin under the action of glutathione S-transferases. Recently, GSH has also been linked to the efflux of other classes of agents such as anthracyclines via the action of the multidrug resistance-associated protein (MRP). In addition to drug detoxification, GSH enhances cell survival by functioning in antioxidant pathways that reduce reactive oxygen species, and maintain cellular thiols (also known as non-protein sulfhydryls (NPSH)) in their reduced states. See, e.g., Kigawa J, et al. Gamma-glutamyl cysteine synthetase up-regulates glutathione and multidrug resistance-associated protein in patients with chemoresistant epithelial ovarian cancer. Clin. Cancer Res. 4:1737-1741 (1998).

Cysteine, another important NPSH, as well as glutathione are also able to prevent DNA damage by radicals produced by ionizing radiation or chemical agents. Cysteine concentrations are typically much lower than GSH when cells are grown in tissue culture, and the role of cysteine as an in vivo cytoprotector is less well-characterized. However, on a molar basis cysteine has been found to exhibit greater protective activity on DNA from the side-effect(s) of radiation or chemical agents. Furthermore, there is evidence that cysteine concentrations in tumor tissues can be significantly greater than those typically found in tissue culture.

A number of studies have examined GSH levels in a variety of solid human tumors, often linking these to clinical outcome See, e.g., Hochwald, S. N., et al. Elevation of glutathione and related enzyme activities in high-grade and metastatic extremity soft tissue sarcoma. American Surg. Oncol. 4:303-309 (1997); Ghazal-Aswad, S., et al. The relationship between tumour glutathione concentration, glutathione S-transferase isoenzyme expression and response to single agent carboplatin in epithelial ovarian cancer patients. Br. J. Cancer 74:468-473 (1996); Berger, S. J., et al. Sensitive enzymatic cycling assay for glutathione: Measurement of glutathione content and its modulation by buthionine sulfoximine in vivo and in vitro human colon cancer. Cancer Res. 54:4077-4083 (1994). Wide ranges of tumor GSH concentrations have been reported, and in general these have been greater (i.e., up to 10-fold) in tumors compared to adjacent normal tissues. Most researchers have assessed the GSH content of bulk tumor tissue using enzymatic assays, or GSH plus cysteine using HPLC.

In addition, cellular thiols/non-protein sulfhydryls (NPSH), e.g., glutathione, have also been associated with increased tumor resistance to therapy by mechanisms that include, but are not limited to: (i) conjugation and excretion of cancer treating agents; (ii) direct and indirect scavenging of reactive oxygen species (ROS) and reactive nitrogen species (RNS); and (iii) maintenance of the “normal” intracellular redox state. Low levels of intracellular oxygen within tumor cells (i.e., tumor hypoxia) caused by aberrant structure and function of the associated tumor vasculature, has also been shown to be associated with chemotherapy therapy-resistance and biologically-aggressive malignant disease. Oxidative stress, commonly found in regions of intermittent hypoxia, has been implicated in regulation of glutathione metabolism, thus linking increased NPSH levels to tumor hypoxia. Therefore, it is also important to characterize both NPSH expression and its relationship to tumor hypoxia in tumors and other neoplastic tissues.

The heterogeneity of NPSH levels was examined in multiple biopsies obtained from patients with cervical carcinomas who were entered into a study investigating the activity of cellular oxidation and reduction levels (specifically, hypoxia) on the response to radical radiotherapy. See, e.g., Fyles, A., et al. (Oxygenation predicts radiation response and survival in patients with cervix cancer. Radiother. Oncol. 48:149-156 (1998). The major findings from this study were that the intertumoral heterogeneity of the concentrations of GSH and cysteine exceeds the intratumoral heterogeneity, and that cysteine concentrations of approximately 21 mM were found in some samples, confirming an earlier report by Guichard, et al. (Glutathione and cysteine levels in human tumour biopsies. Br. J. Radiol. 134:63557-635561 (1990)). These levels of cysteine are much greater than those typically seen in tissue culture, suggesting that cysteine might exert a significant radioprotective activity in cervical carcinomas and possibly other types of cancer.

There is also extensive literature showing that elevated cellular glutathione levels can produce drug resistance in experimental models, due to drug detoxification or to the antioxidant activity of GSH. In addition, radiation-induced DNA radicals can be repaired non-enzymatically by GSH and cysteine, indicating a potential role for NPSH in radiation resistance. While cysteine is the more effective radioprotective agent, it is usually present in lower concentrations than GSH. Interestingly, under fully aerobic conditions, this radioprotective activity appears to be relatively minor, and NPSH competes more effectively with oxygen for DNA radicals under the hypoxic conditions that exist in some solid tumors, which might play a significant role in radiation resistance.

Radiotherapy has traditionally been a major treatment modality for cervical carcinomas. Randomized clinical trials (Rose, D., et al. Concurrent cisplatin-based radiotherapy and chemotherapy for locally advanced cervical carcinoma. New Engl. J. Med. 340:1144-1153 (1999)) show that patient outcome is significantly improved when radiation therapy is combined with cisplatin-based chemotherapy, and combined modality therapy is now widely being utilized in treatment regimens. It is important to establish the clinical relevance of GSH and cysteine levels to drug and radiation resistance because of the potential to modulate these levels using agents such as buthionine sulfoximine; an irreversible inhibitor of γ-glutanylcysteine synthetase that can produce profound depletion of GSH in both tumor and normal tissues. See, e.g., Bailey, T., et al. Phase I clinical trial of intravenous buthionine sulfoximine and melphalan: An attempt at modulation of glutathione. J. Clin. Oncol. 12:194-205 (1994). Evaluation of GSH concentrations have reported elevated tumor GSH relative to adjacent normal tissue, and intertumoral heterogeneity in GSH content.

Koch and Evans (Cysteine concentrations in rodent tumors: unexpectedly high values may cause therapy resistance. Int. J. Cancer 67:661-667 (1996)) have shown that cysteine concentrations in established tumor cell lines can be much greater when these are grown as in vivo tumors, as compared to the in vitro values, suggesting that cysteine might play a more significant role in therapy resistance than previously considered. Although relatively few studies have reported on cysteine levels in human cancers, an earlier HPLC-based study of cervical carcinomas by Guichard, D. G., et al. (Glutathione and cysteine levels in human tumour biopsies. Br. J. Radiol. 134:63557-635561 (1990) reported cysteine concentrations greater than 1 mM in a significant number of cases. Thus, the fact that the variability in cysteine levels is greater than that for GSH suggests that these two thiols are regulated differently in tumors. By way of non-limiting example, the inhibition of γ-glutamylcysteine synthetase with the intravenous administration of buthionine sulfoximine (BSO) could result in elevated cellular levels of cysteine, due to the fact that the α-glutamylcysteine synthetase is not being utilized for GSH de novo synthesis. Similar to GSH, cysteine possesses the ability to repair radiation-induced DNA radicals and cysteine also has the potential to detoxify cisplatin; a cytotoxic agent now routinely combined with radiotherapy to treat locally-advanced cervical carcinomas.

(b) Glutaredoxin

Glutaredoxin (Grx), like thioredoxin (Trx), are members of the thioredoxin superfamily that mediate disulfide exchange via their Cys-containing catalytic sites. While glutaredoxins mostly reduce mixed disulfides containing glutathione, thioredoxins are involved in the maintenance of protein sulfhydryls in their reduced state via disulfide bond reduction. See, e.g., Print, W. A., et al. The role of the thioredoxin and glutaredoxin pathways in reducing protein disulfide bonds in the Escherichia coli cytoplasm. J. Biol. Chem. 272:15661-15667 (1996). The reduced form of thioredoxin is generated by the action of thioredoxin reductase; whereas glutathione provides directly the reducing potential for regeneration of the reduced form of glutaredoxin.

Glutaredoxins are small redox enzymes of approximately 100 amino acid residues, which use glutathione as a cofactor. Glutaredoxins are oxidized by substrates, and reduced non-enzymatically by glutathione. In contrast to thioredoxins, which are reduced by thioredoxin reductase, oxidized glutathione is regenerated by glutathione reductase. Together these components comprise the glutathione system. See, e.g., Holmgren, A. and Fernandes, A. P., Glutaredoxins: glutathione-dependent redox enzymes with functions far beyond a simple thioredoxin backup system. Antioxid. Redox. Signal. 6:63-74 (2004).

Glutaredoxins basically function as electron carriers in the glutathione-dependent synthesis of deoxyribonucleotides by the enzyme ribonucleotide reductase. Like thioredoxin, which functions in a similar way, glutaredoxin possesses an active catalytic site disulfide bond. It exists in either a reduced or an oxidized form where the two cysteine residues are linked in an intramolecular disulfide bond. Human proteins containing this domain include: glutaredoxin thioltransferase (GLRX); glutaredoxin 2 (GLRX2); thioredoxin-like 2 (GLRX3); GLRX5; PTGES2; and TXNL3. See, e.g., Nilsson, L. and Foloppe, N., The glutaredoxin -C-P-Y-C-motif: influence of peripheral residues. Structure 12:289-300 (2004).

At least two glutaredoxin proteins exist in mammalian cells (12 or 16 kDa), and glutaredoxin, like thioredoxin, cycles between disulfide and dithiol forms. The conversion of glutaredoxin from the disulfide form (oxidized) to the dithiol (reduced) form is catalyzed non-enzymatically by glutathione. In turn, glutathione cycles between a thiol form (glutathione) that can reduce glutaredoxin and a disulfide form (glutathione disulfide); glutathione reductase enzymatically reduces glutathione disulfide to glutathione.

(iii) The Thioredoxin Reductase/Thioredoxin System

The thioredoxin system is comprised of thioredoxin reductase (TrxR) and its main protein substrate, thioredoxin (Trx), where the catalytic site disulfide of Trx is reduced to a dithiol by TrxR at the expense of NADPH. The thioredoxin system, together with the glutathione system (comprising NADPH, the flavoprotein glutathione reductase, glutathione, and glutaredoxin), is regarded as a main regulator of the intracellular redox environment, exercising control of the cellular redox state and antioxidant defense, as well as governing the redox regulation of several cellular processes. The system is involved in direct regulation of: (i) several transcription factors; (ii) apoptosis (i.e., programmed cell death) induction; and (iii) many metabolic pathways (e.g., DNA synthesis, glucose metabolism, selenium metabolism, and vitamin C recycling). See, e.g., Amer, E. S. J., et al. Physiological functions of thioredoxin and thioredoxin reductase. Eur. J. Biochem. 267:6102-6109 (2000).

### Thioredoxin Reductase (TrxR)

The mammalian thioredoxin reductases (TrxRs) are enzymes belonging to the avoprotein family of pyridine nucleotide-disulfide oxidoreductases that includes lipoamide dehydrogenase, glutathione reductase, and mercuric ion reductase. Members of this family are homodimeric proteins in which each monomer includes an FAD prosthetic group, an NADPH binding site and an active site containing a redox-active disulfide. Electrons are transferred from NADPH via FAD to the active-site disulfide of Trx, which then reduces the substrate. See, e.g., Williams, C. H., Chemistry and Biochemistry of Flavoenzymes (Muller, F., ed.), pp. 121-211, CRC Press, Boca Raton (1995).

TrxRs are named for their ability to reduce oxidized thioredoxins (Trxs), a group of small (i.e., 10-12 kDal), ubiquitous redox-active peptides that undergoes reversible oxidation/reduction of two conserved cysteine (Cys) residues within the catalytic site. The mammalian TrxRs are selenium-containing flavoproteins that possess: (i) a conserved -Cys-Val-Asn-Val-Gly-Cys-catalytic site; (ii) an NADPH binding site; and (iii) a C-terminal Cys-Selenocysteine sequence that communicates with the catalytic site and is essential for its redox activity. See, e.g., Powis, G. Monofort, W. R. Properties and biological activities of thioredoxins. Ann. Rev. Pharmacol. Toxicol. 41:261-295 (2001). These proteins exist as homodimers and undergo reversible oxidation/reduction. The activity of TrxR is regulated by NADPH, which in turn is produced by glucose-6-phosphate dehydrogenase (G6DP), the rate-limiting enzyme of the oxidative hexose monophosphate shunt (HMPS; also known as the pentose phosphate pathway). Two human TrxR isozyme genes have been cloned: (i) the gene for human TrxR-1 located on chromosome 12q23-q24.1 encoding a 54 Kda enzyme that is found predominantly in the cytoplasm; and (ii) the gene for human TrxR-2 located on chromosome 22q11.2 encoding a 56 Kda enzyme the possesses a 33-amino-acid N-terminal extension identified as a mitochondrial import sequence. See, e.g., Powis, G. Monofort, W. R. Properties and biological activities of thioredoxins. Ann. Rev. Pharmacol. Toxicol. 41:261-295 (2001). A third isoform of TrxR, designated (TGR) is a Trx and glutathione reductase localized mainly in the testis, has also been identified. See, e.g., Sun, Q. A., et al. Selenoprotein oxidoreductase with specificity for thioredoxin and glutathione systems. Proc. Natl. Acad. Sci. USA 98:3673-3678 (2001). Additionally, both mammalian cytosolic TrxR-1 and mitochondrial TrxR-2 have alternative splice variants. In humans, five different 5′ cDNA variants have been reported, with one of the splice variants comprising a 67 kDa protein with an N-terminal elongation, instead of the common 55 kDa. The physiological functions of these TrxR splice variants have yet to be elucidated. See, e.g., Sun, Q. A., et al. Heterogeneity within mammalian thioredoxin reductases: evidence for alternative exon splicing. J. Biol. Chem. 276:3106-3114 (2001).

Some of the major functions of mammalian Trx proteins are to supply reducing equivalents to enzymes such as ribonucleotide reductase and thioredoxin peroxidase, as well as (through thiol-disulphide exchange) to reduce key Cys residues in certain transcription factors, resulting in their increased binding to DNA and altered gene transcription. Mammalian Trxs have also been shown to function as cell growth factors and to inhibit apoptosis. Since TrxRs are the only class of enzymes known to reduce oxidized Trx, it is possible that alterations in TrxR activity may regulate some of the activities of Trxs. In addition to Trxs, other endogenous substrates have been demonstrated for TrxRs, including, but not limited to: lipoic acid, lipid hydroperoxides, the cytotoxic peptide NK-lysin, vitamin K₃, dehydroascorbic acid, the ascorbyl free radical, and the tumor-suppressor protein p53. See, e.g., Mustacich, D., Powis, G. Thyrodoxin Reductase. Biochem. J. 346:1-8 (2000). However, the physiological role that TrxRs play in the reduction of most of these substrates has not been fully elucidated.

Mammalian TrxRs are promiscuous enzymes capable of reducing Trxs of different species, proteins such as NK lysin and p53, a variety of physiological substrates (see, e.g., May, J. M., Cobb, C. E., et al. J. Biol. Chem. 273:23039-23045 (1998), as well as several exogenous compounds (see, e.g., Kumar, S., Bjornstedt, M., Holmgren, A. Eur. J. Biochem. 207:435-439 (1992). One suggested catalytic mechanism for human TrxR is that the C-terminal end of the protein is flexible, allowing the -Cys-SeCys-Gly moiety to carry reducing equivalents from the conserved active-site Cys residues to the substrate. See, e.g., Gromer, S., Wissing, J., et al. Biochem. J. 332:591-592 (1998).

The involvement of TrxR in biological functions such as cell growth and protection from oxidative stress has, to date, centred around its role as a reductant for Trx. Further studies are needed to determine whether TrxR has biological functions that are not directly mediated by reduction of Trx.

### Cell Replication

Trx, a physiological substrate of TrxRs, has been shown to play an important role in regulating cell growth and inhibiting apoptosis. See, e.g., Baker, A., Payne, C. M., Briehl, M. M., Powis, G. Cancer Res. 57:5162-5167 (1997). Trx has to be in a reduced form in order to exert these effects, and mutant redox-inactive forms of Trx are unable to stimulate cell growth or inhibit apoptosis. The only known mechanism for the reduction of Trx is through NADPH-dependent reduction by TrxR.

Inhibiting TrxR activity to below normal levels is associated with inhibited cell growth. Several in vitro inhibitors of TrxR have been reported and, although many of these compounds only inhibit the reduced form of TrxR, it is likely that TrxR will be sensitive to these inhibitors in vivo, since TrxR is expected to exist predominantly in the reduced form due to the presence of cytosolic NADPH concentrations that are greater than the Kₘ of TrxR for NADPH. See, e.g., Gromer, S., Arscott, L. D., et al. J. Biol. Chem. 273:20096-20101 (1998). Two such inhibitors of TrxR are the anti-tumour quinones doxorubicin and diaziquone; wherein treatment of cells with either of these compounds leads to secondary inhibition of ribonucleotide reductase and inhibition of cell growth. See, e.g., Hofman, E. R., Boyanapalli, M., et al. Mol. Cell. Biol. 18:6493-6504 (1998).

Protection Against Oxidative Stress

The continual formation of low levels of reactive oxygen species (ROS) is part of normal O₂ metabolism; however, increased production of ROS, or a functional decrease in one or more of the protective systems present in the cell, can result in unrepaired macromolecular damage (i.e., oxidation of protein thiols), which may then lead to pathological processes, including apoptosis. See, e.g., Zhivotovsky, B., Orrenius, S., et al. Nature (London) 391:449-450 (1998). Trx has been shown to prevent apoptosis in cells treated with agents known to produce ROS. By way of example, the levels of TrxR-1 mRNA and Trx mRNA are increased in the lungs of newborn baboons exposed to air or O₂ breathing, and increases in TrxR-1 and Trx mRNA are also observed in adult baboon lung explants in response to 95% O₂. It has been suggested that these increases in gene expression for TrxR1 and Trx play a protective role against O₂ breathing in the mammalian lung. There have also been reports that TrxR is highly expressed on the surface of human keratinocytes and melanocytes, where it has been suggested to provide the skin's first line of defence against free radicals generated in response to UV light. See, e.g., Schallreuter, K. U., Wood, J. M. Cancer Lett. 36:297-305 (1997).

### Cancer Involvement

It has been suggested, based on purification yields, that the level of TrxR in tumor cells is 10-fold or more greater than in normal tissues. See, e.g., Tamura, T., Stadtman, T. C. Proc. Natl. Acad. Sci. U.S.A. 93:1006-1011 (1996). TrxR has also been reported to be elevated in human primary melanoma and to show a correlation with invasiveness. See, e.g., Fuchs, J. Arch. Dermatol. 124:849-850 (1998). The Trx system is as an electron donor for ribonucleotide reducatse, which is frequently greatly over-expressed in cancer cells potentially leading to expanded and inbalanced deoxynucletide pools which are mutagenic, which may accelerate the development of the malignant phenotype by major genetic rearrangements, gene amplifications, total loss of growth control and therapy resistance. It is clearly evident that the Trx system plays a central role in established cancers particularly for distant metastasis and angiogenesis. A recent study utilizing TrxR-1 knock-down in tumor cells intriguingly demonstrated a necessity of TrxR-1 expression for cancer cell growth and tumor development. See, e.g., Yoo, M. H., Xu, X. M., et al. Thioredoxin reductase 1 deficiency reverses tumor phenotype and tumorigenicity of lung carcinoma cells. J. Biol. Chem. 281:13005-13008 (2006).

### Thioredoxin (Trx)

Thioredoxins (Trxs) are proteins that act as antioxidants by facilitating the reduction of other proteins by cysteine thiol-disulfide exchange. While glutaredoxins mostly reduce mixed disulfides containing glutathione, thioredoxins are involved in the maintenance of protein sulfhydryls in their reduced state via disulfide bond reduction. Thiol-disulfide exchange is a chemical reaction in which a thiolate group (S) attacks a sulfur atom of a disulfide bond (—S—S—). The original disulfide bond is broken, and its other sulfur atom is released as a new thiolate, thus carrying away the negative charge. Meanwhile, a new disulfide bond forms between the attacking thiolate and the original sulfur atom. The transition state of the reaction is a linear arrangement of the three sulfur atoms, in which the charge of the attacking thiolate is shared equally. The protonated thiol form (—SH) is unreactive (i.e., thiols cannot attack disulfide bonds, only thiolates). In accord, thiol-disulfide exchange is inhibited at low pH (typically, <8) where the protonated thiol form is favored relative to the deprotonated thiolate form. The pKₐ of a typical thiol group is approximately 8.3, although this value can vary as a function of the environment. See, e.g., Gilbert, H. F., Molecular and cellular aspects of thiol-disulfide exchange. Adv. Enzymol. 63:69-172 (1990); Gilbert, H. F., Thiol/disulfide exchange equilibria and disulfide bond stability. Meth. Enzymol. 251:8-28 (1995).

Thiol-disulfide exchange is the principal reaction by which disulfide bonds are formed and rearranged within a protein. The rearrangement of disulfide bonds within a protein generally occurs via intra-protein thiol-disulfide exchange reactions; a thiolate group of a cysteine residue attacks one of the protein's own disulfide bonds. This process of disulfide rearrangement (known as disulfide shuffling) does not change the number of disulfide bonds within a protein, merely their location (i.e., which cysteines are actually bonded). Disulfide reshuffling is generally much faster than oxidation/reduction reactions, which actually change the total number of disulfide bonds within a protein. The oxidation and reduction of protein disulfide bonds in vitro also generally occurs via thiol-disulfide exchange reactions. Typically, the thiolate of a redox reagent such as glutathione or dithiothreitol (DTT) attacks the disulfide bond on a protein forming a mixed disulfide bond between the protein and the reagent. This mixed disulfide bond when attacked by another thiolate from the reagent, leaves the cysteine oxidized. In effect, the disulfide bond is transferred from the protein to the reagent in two steps, both thiol-disulfide exchange reactions.

The mammalian thioredoxins (Trxs) are a family of 10-12 kDa proteins that contain a highly conserved -Trp-Cys-Gly-Pro-Cys-Lys-catalytic site. See, e.g., Nishinaka, Y., et al. Redox control of cellular functions by thioredoxin: A new therapeutic direction in host defense. Arch. Immunol. Ther. Exp. 49:285-292 (2001). The active site sequences is conserved from Escherichia coli to humans. Thioredoxins in mammalian cells possess >90% homology and have approximately 27% overall homology to the E. coli protein.

Two principal forms of thioredoxin (Trx) have been cloned. Trx-1 is a 105-amino acid protein. In almost all (>99%) of the human form of Trx-1, the first methionine (Met) residue is removed by an N-terminus excision process (see, e.g., Giglione, C., et al. Protein N-terminal methionine excision. Cell. Mol. Life Sci. 61:1455-1474 (2004), and therefore the mature protein is comprised of a total of 104-amino acid residues from the N-terminal valine (Val) residue. Trx-1 is typically localized in the cytoplasm, but it has also been identified in the nucleus of normal endometrial stromal cells, tumor cells, and primary solid tumors.

Trx-2 is a 166-amino acid residue protein that contains a 60-amino acid residue N-terminal translocation sequence that directs it to the mitochondria. See, e.g., Spyroung, M., et al. Cloning and expression of a novel mammalian thioredoxin. J. Biol. Chem. 272: 2936-2941 (1997). Trx-2 is expressed uniquely in mitochondria, where it regulates the mitochondrial redox state and plays an important role in cell proliferation. Trx-2-deficient cells fall into apoptosis via the mitochondria-mediated apoptosis signaling pathway. See, e.g., Noon, L., et al. The absence of mitochondrial thioredoxin-2 causes massive apoptosis and early embryonic lethality in homozygous mice. Mol. Cell. Biol. 23:916-922 (2003). Trx-2 was found to form a complex with cytochrome c localized in the mitochondrial matrix, and the release of cytochrome c from the mitochondria was significantly enhanced when expression of Trx-2 was inhibited. The overexpression of Trx-2 produced resistance to oxidant-induced apoptosis in human osteosarcoma cells, indicating a critical role for the protein in protection against apoptosis in mitochondria. See, e.g., Chen, Y., et al. Overexpressed human mitochondrial thioredoxin confers resistance to oxidant-induced apoptosis in human osteosarcoma cells. J. Biol. Chem. 277:33242-33248 (2002).

As both Trx-1 and Trx-2 are known regulators of the manifestation of apoptosis under redox-sensitive capases, their actions may be coordinated. However, the functions of Trx-1 and Trx-2 do not seem to be capable of compensating for each other completely, since Trx-2 knockout mice were found be embryonically lethal. See, e.g., Noon, L., et al. The absence of mitochondrial thioredoxin-2 causes massive apoptosis and early embryonic lethality in homozygous mice. Mol. Cell. Biol. 23:916-922 (2003). Moreover, the different subcellular locations of both the thioredoxin reductase (TrxR) and thioredoxin (Trx) subtypes suggest that the cytoplasmic and mitochondrial systems may play different roles within cells. See, e.g., Powis, G. and Monofort, W. R. Properties and biological activities of thioredoxins. Ann. Rev. Pharmacol. Toxicol. 41:261-295 (2001).

While Trx itself is not mutagenic, the Trx system is involved in antioxidant defense and probably in prevention of cancer via the removal of carcinogenic oxidants or by repair of oxidized proteins. Similarly repair of mutagenic DNA lesions by Trx system-dependent nucleotide excision repair and ribonucleotide reductase may protect from cancer. In theory, the Trx system as an electron donor for ribonucleotide reducatse, which is often greatly over-expressed in cancer cells. This over-expression may potentially lead to an expanded and inbalanced deoxynucletide pools which is mutagenic and may accelerate the development of the malignant phenotype by major genetic rearrangements, gene amplifications, total loss of growth control, and resistance to the selected therapy.

Thioredoxin (Trx) expression is frequently markedly increased in a variety of human malignancies including, but not limited to, lung cancer, colorectal cancer, cervical cancer, hepatic cancer, pancreatic cancer, and adenocarcinoma. See, e.g., Arne, E. S. J., Holmgren, A. The thirodoxin system in cancer. Sem. Cancer Biol. 16:420-426 (2006). In addition, Trx over-expression has also been associated with aggressive tumor growth. See, e.g., Id. This increase in expression level is likely related to changes in the Trx protein structure and function. For example, in pancreatic ductal carcinoma tissue, Trx levels were found to be elevated in 24 of 32 cases, as compared to normal pancreatic tissue; whereas glutaredoxin levels were increased in 29 of 32 of the cases. See, e.g., Nakamura, H., et al. Expression of thioredoxin and glutaredoxin, redox-regulating proteins, in pancreatic cancer. Cancer Detect. Prev. 24:53-60 (2000). Similarly, tissue samples of primary colorectal cancer or lymph node metastases had significantly higher Trx-1 levels than normal colonic mucosa or colorectal adenomatous polyps. See, e.g., Raffel, J., et al. Increased expression of thioredoxin-1 in human colorectal cancer is associated with decreased patient survival. J. Lab. Clin. Med. 142:46-51 (2003).

In two recent studies, Trx expression was associated with aggressive tumor growth and poorer prognosis. In a study of 102 primary non-small cell lung carcinomas, tumor cell Trx expression was measured by immunohistochemistry of formalin-fixed, paraffin-embedded tissue specimens. See, e.g., Kakolyris, S., et al. Thioredoxin expression is associated with lymph node status and prognosis in early operable non-small cell lung cancer. Clin. Cancer Res. 7:3087-3091 (2001). The absence of Trx expression was significantly associated with lymph node-negative status (P=0.004) and better outcomes (P<0.05) and was found to be independent of tumor stage, grade, or histology. The investigators also concluded that these results were consistent with the proposed role of Trx as a growth promoter in some human cancers, and overexpression may be indicative of a more aggressive tumor phenotype (hence the association of Trx overexpression with nodal positivity and poorer outcomes). In another study of 37 patients with colorectal cancer, Trx-1 expression tended to increase with higher Dukes stage (P=0.077) and was significantly correlated with reduced survival (P=0.004).

The relationship between TrxR activity and tumor growth is less clear. Tumor cells may not need to increase expression of the TrxR enzyme, although its catalytic activity may be increased functionally. For example, human colorectal tumors were found to have 2-times higher TrxR activity than normal colonic mucosa. See, e.g., Mustacich, D. and Powis, G., Thioredoxin reductase. Biochem. J. 346:1-8 (2000). TrxR has also been reported to be elevated in human primary melanoma and to show a correlation with invasiveness. See, e.g., Schallreuter, K. U., et al. Thioredoxin reductase levels are elevated in human primary melanoma cells. Int. J. Cancer 48:15-19 (1991). Further evaluations relating TrxR enzyme levels and catalytic activity with cancer stage and outcome are required to fully elucidate this relationship.

Similarly, several lines of evidence suggest that thioredoxin (Trx) may also be necessary, but is not sufficient in toto, for conferring cancer cell resistance to many chemotherapeutic drugs. This evidence includes, but is not limited to: (i) the resistance of adult T-cell leukemia cell lines to doxorubicin and ovarian cancer cell lines to cisplatin has been associated with increased intracellular Trx-1 levels; (ii) hepatocellular carcinoma cells with increased Trx-1 levels were less sensitive to cisplatin (but not less sensitive to doxorubicin or mitomycin C); (iii) Trx-1 mRNA and protein levels were increased by 4- to 6-fold in bladder and prostate cancer cells made resistant to cisplatin, but lowering Trx-1 levels with an antisense plasmid restored sensitivity to cisplatin and increased sensitivity to several other cytotoxic drugs; (iv) Trx-1 levels were elevated in cisplatin-resistant gastric and colon cancer cells; and (v) stable transfection of fibrosarcoma cells with Trx-1 resulted in increased cisplatin resistance. See, e.g., Biaglow, J. E. and Miller, R. A., The thioredoxin reductase/thioredoxin system. Cancer Biol. Ther. 4:6-13 (2005).

Glutathione may also play a role in resistance to the effects of cancer drugs. Glutathione-S-transferases catalyze the conjugation of glutathione to many electrophilic compounds, and can be upregulated by a variety of cancer drugs. Glutathione-S-transferases possess selenium-independent peroxidase activity. Mμ also has been shown to possess glutaredoxin activity. Some agents are substrates for glutathione-S-transferase and are directly inactivated by glutathione conjugation, thus leading to resistance. Examples of enzyme substrates include melphalan, carmustine (BCNU), and nitrogen mustard. In a panel of cancer cell lines, glutathione-S-transferase expression was correlated inversely with sensitivity to alkylating agents. Other drugs that upregulate glutathione-S-transferase may become resistant, because the enzyme also inhibits the MAP kinase pathway. These agents require a functional MAP kinase, specifically JNK and p38 activity, to induce an apoptotic response. See, e.g., Townsend, D. M. and Tew, K. D., The role of glutathione-S-transferase in anti-cancer drug resistance. Oncogene 22:7369-7375 (2003).

### V. Pharmacology of the Sulfur-Containing, Amino Acid-Specific Small Molecules of the Present Invention

The sulfur-containing, amino-acid specific small molecules of the present invention include the following molecules: (i) 2,2′-dithio-bis-ethane sulfonate; (ii) the metabolite of 2,2′-dithio-bis-ethane sulfonate, known as 2-mercapto ethane sulfonate; and (iii) additional molecules comprising 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, and -Homocysteine-Glu; and

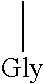

pharmaceutically-acceptable salts thereof.

Most notably for purposes of the present invention, Tavocept (also know in the literature as 2,2′-dithiobis ethane sulfonate; BNP7787, dimesna) and the metabolite of Tavocept, 2-mercapto ethane sulfonate, act to selectively reduce the toxicity of certain antineoplastic agents in vivo. 2-mercapto-ethane sulfonate conjugated as a disulfide with a substituent group comprising of one or more amino acid residues are known herein as Tavocept-derived heteroconjugates.

Tavocept is the physiological auto-oxidation dimer of mesna. Mesna (I) and Tavocept (II) have the following molecular structures:

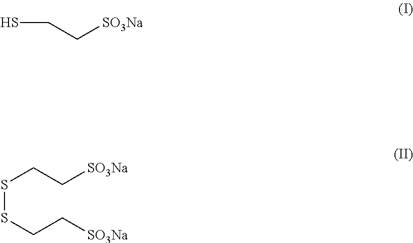

The pharmaceutical chemistry of the aforementioned compounds indicates that the terminal sulfhydryl group of mesna (and to a lesser extent the disulfide linkage in dimesna) acts as a substitution group for the terminal hydroxy- or aquo-moiety in the active metabolites of, e.g., platinum complexes. Dimesna requires a metabolic activation, such as by glutathione reductase, to exert its biologically efficacious results. Dimesna also exhibits significantly lower toxicity than mesna. The conversion from the hydroxy- or aquo-moiety to a thioether is favored, particularly under acidic conditions, and results in the formation of a hydrophilic compound of much lower toxicity, which is rapidly eliminated from the body. Since blood plasma is slightly alkaline (pH ˜7.3), the more stable disulfide form is the favored species, and does not readily react with the nucleophilic terminal chlorine in cisplatin or the cyclobutane dicarboxylato moiety of carboplatin. This allows Tavocept to perform its intended beneficial effects on the targeted cancer cells.

The putative mechanisms of the sulfur-containing, amino-acid specific small molecules of the present invention which function to increase the cytotoxic or cytostatic activity of cancer treating agents may involve one or more of several novel pharmacological and physiological factors.

Preferred doses of the sulfur-containing, amino-acid specific small molecules of the present invention range from about 1 g/m² to about 50 g/m², preferably about 5 g/m² to about 40 g/m² (for example, about 10 g/m² to about 30 g/m²), more preferably about 14 g/m² to about 22 g/m², with a most preferred dose of 18.4 g/m².

### A. Mechanisms of Action of the Sulfur-Containing, Amino-Acid Specific Small Molecules of the Present Invention

In brief, Tavocept is a sulfur-containing, amino acid-specific, small molecule that possesses the ability to function as a multi-target modifier and/or modulator of the function of the target molecules of the present invention. Tavocept mediates the non-enzymatic xenobiotic modification of sulfur-containing amino acid residues (e.g., cysteine) on proteins. As an engineered, non-naturally occurring agent (i.e., xenobiotic), Tavocept is autocatalytic and requires no protein cofactor to cause the xenobiotic modification of cysteine, but appears to be specific for cysteine residues located within a particular structural context (i.e., not all cysteines in a protein are so modified). Tavocept-mediated, xenobiotic modification represents a novel mechanism of action for a cancer treating agent and can be compared to a degree with post-translational modifications of cysteine residues in proteins (see, Table 3, below). By way of non-limiting example, an important element of Tavocept's effectiveness as a compound in the treatment of cancer is its selectivity for normal cells versus cancer cells and its absence of interference with the anti-cancer activity of cancer treating agents. In vitro studies demonstrated that Tavocept does not interfere with paclitaxel induced apoptosis, as assessed by PARP cleavage, Bcl-2 phosphorylation, and DNA laddering in human breast, ovarian and lymphoma cancer cell lines. Additionally, Tavocept was shown not to interfere with paclitaxel- and platinum-induced cytotoxicity in human cancer cell lines, which are discussed herein, infra.

The believed mechanisms underlying the absence of interference with anti-cancer activity by Tavocept are multifactorial and, as previously discussed, may involve its selectivity for normal cells versus cancer cells, inherent chemical properties that have minimal impact in normal cells on critical plasma and cellular thiol-disulfide balances, and its interactions with cellular oxidoreductases, which are key in the cellular oxidative/reduction (redox) maintenance systems.

In addition to the absence of interference with anti-cancer activity, results from in vivo studies have shown that Tavocept may elicit the restoration of apoptotic sensitivity in tumor cells through, e.g., thioredoxin- and glutaredoxin-mediated mechanisms and this may be an important element of its effectiveness as a chemotherapeutic agent.

TABLE 3
Cysteine-Specific Protein Modifications

| | | Protein Cofactor(s) |
|---|---|---|
| Modification | Specificity | Required? |
| Tavocept-mediated | Cysteines near or in α-helices, with nearby | No |
| xenobiotic modification | residues to accept the cysteine thiol proton | |
| | and stabilize the cysteinyl thiolate | |
| Glutathionylation | May involve cysteines with altered pKa's | Can be autocatalytic |
| | (vicinal to lysine, arginine or histidine) | or protein catalyzed |
| Nitrosylation | Possible specificity at the tertiary | No |
| | environment level around cysteine | |
| Prenylation | Varied sequences around target cysteine | Yes |
| (Farnesylation, | with a CaaX motif (a = aliphatic amino | |
| geranylgeranylation) | acid; X = one of several amino acids | |
| | depending on protein) | |
| Palmitoylation | Varied Sequences | Can be autocatalytic |
| | | or protein catalyzed |

### B. Specificity of Tavocept-Mediated, Xenobiotic Modification of Cysteines

In order to react with Tavocept, a cysteine residue requires certain physico-chemical characteristics, these include: (i) accessibility; (ii) proximity to a hydrogen bond donor (facilitating thiolate formation); (iii) a shielded or hydrophobic microenvironment (to stabilize the thiolate); and (iv) location within or near α-helix (cysteines within β-strands do not appear to react). A number of important target molecules contain Tavocept-reactive cysteine moieties. These molecular targets include, but are not limited to, anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif comprising the physicochemical characteristics described above and in subsequent paragraphs.

### C. Cellular Consequences of Tavocept-Mediated, Xenobiotic Modification of Cysteines

The effect of Tavocept-mediated xenobiotic modification on molecular targets that are involved in regulating cell growth and cell survival, and thereby impact cancer and other diseases, manifests itself in distinct, target-specific ways that are correlated to the role of the cysteine residue that undergoes xenobiotic modification, including:
Modification of non-catalytic cysteines important in protein function/structure.
Functioning as an alternative substrate/inhibitor (e.g., Trx, Grx, APN, GGT) resulting in impaired enzyme activity
Disruption of active site structure (e.g., Prx) resulting in impaired enzyme activity
Disruption/blocking of cofactor binding site resulting in enzyme inhibition (e.g., ALK)
Modification of non-active site cysteine(s) resulting in enzyme inhibition (e.g., MET)

### D. Modification/Modulation of Cysteine Function as a Drug-Development Strategy

As yet, no drugs have been approved for use in humans with a reported mechanism of action involving the ability to covalently modify proteins on cysteine residues and, subsequently, modify/modulate protein function. Since most proteins contain cysteine, the development of agents that specifically target physico-chemically distinct cysteine residues is an area of therapy that has been, to date, largely ignored. Additionally, with the growing recognition of the importance of protein glutathionylation/deglutathionylation in cell growth and proliferation, it is clear that modulation of protein cysteine residue functioning is a viable target for development of agents in a wide range of therapeutic areas. The sulfur-containing, amino acid-specific small molecules of the present invention represent novel, first in class, cancer treating agents that are shown herein, specifically and unequivocally, to work through a mechanism of action involving cysteine modification of proteins that directly translates to impaired, inhibited, or altered protein function.

E. Examples of Non-Cancerous Diseases with Abnormal Expression of Specified Molecular Targets and/or Abnormal Biochemical Function

### Rheumatoid Arthritis

In rheumatoid arthritis (RA), the synovial membrane exhibits molecular features common to cancer including hyperplasia and a tendency towards invasiveness. Swanson et al., at Stanford's Division of Immunology and Rheumatology, studied a mouse model of human rheumatoid arthritis (the murine collagen-induced arthritis model) and conducted studies on human cell and tissue samples. They observed that rheumatoid arthritis patients highly express activated EGFR in their synovial tissue. They also found that vascular endothelial and fibroblast cells from rheumatoid arthritis patients express the epidermal growth factor receptor (EGFR). Additionally, the EGFR targeted inhibitor, Erlotinib, was show to inhibit proliferation of the human endothelial cell line HUVEC in vitro. This finding is important because in rheumatoid arthritis, endothelial cells line the blood vessels found in the synovial membrane. As rheumatoid arthritis progresses, neovascularization or neoangiogenesis occurs providing nourishment for the continued growth of the synovial membrane (synovium). Compounds that inhibit EGFR are expected to inhibit the formation of new blood vessels in the synovium and to serve as effective anti-rheumatoid arthritis treatment agents. See, e.g., Swanson, et al., Inhibition of Epidermal Growth Factor Receptor Tyrosine Kinase Ameliorates Collagen-Induced Arthritis. J. Immunol. 188: 3513-3521 (2012); Yuan, et al., Epidermal Growth Factor Receptor as a therapeutic target in rheumatoid arthritis. Clin. Rheumatol. 32 (3):289-29 (2013).

### Acquired Immune Deficiency Syndrome (AIDS)

Levels of thioredoxin are elevated in human immunodeficiency virus 1 (HIV-1) infected patients that have the acquired immune deficiency syndrome (AIDS) phenotype. Thioredoxin (Trx) and the Trx family protein, protein disulfide isomerase (PDI), have been implicated as thiol-disulfide modulating proteins important during the process of HIV-1 infection. Both Trx and PDI are expressed on cell membranes, which may facilitate their contact with and modulation of HIV-1 during the process of infection. Elevated thioredoxin is thought to be correlated to increased HIV-1 entry into macrophages. See, e.g., Stantchev, et al., Cell-type specific requirements for thiol/disulfide exchange during HIV-1 entry and infection. Retrovirology 9(97):1-15 (2012). It has been shown that thioredoxin cleaves the Cys296-Cys331 disulfide bond present in the HIV envelope glycoprotein (gp120), resulting in gp120 refolding/reorganization, a process which activates the protein and facilitates infection. Subsequent to the thioredoxin-mediated disulfide bond cleavage, gp120 fuses with the cell membrane and infects the cell. See Azimi et al, Disulfide Bond That Constrains the HIV-1 gp120 V3 Domain is Cleaved by Thioredoxin, J. Biol. Chem. 285 (51):40072-40080 (2010). Additionally, Nakamura et al., have suggested that elevated Trx directly impacts survival of AIDS patients due to Trx-mediated inhibition of neutrophil migration which is correlated with lowered life expectancy. Nakamura et al. also demonstrated that human Trx injected into mouse models inhibited the chemotactic movement of neutrophils towards injected lipopolysaccharide. See Nakamura et al, Chronic elevation of plasma thioredoxin:inhibition of chemotaxis and curtailment of life expectancy in AIDS, Proc. Natl. Acad. Sci. U.S.A. 98(5):2688-2693(2001).

### Alzheimer's Disease

One widely accepted mechanism for development of Alzheimer's disease involves production of elevated levels of the amyloid β (Aβ) peptide which is mediated by the secretase proteins (α-secretase, β-secretase, and γ-secretase). Concurrently, elevated amyloid β (Aβ) peptide has been reported to be correlated to peroxiredoxin 1 levels, and peroxiredoxin appears to modulate γ-secretase expression. See, e.g., Lee, et al., Peroxiredoxin 1 regulates the component expression of γ-secretase complex causing the Alzheimer's disease. Lab. Anim. Res. 27(4):293-299 (2011); De Strooper, et al., The secretases: enzymes with therapeutic potential in Alzheimer disease. Nat. Rev. Neurol. 6(2):99-107 (2010). A post-mortem study also indicated that peroxiredoxin 1 is elevated in the brains of Alzheimer's disease patients. See e.g., Cumming et al, Protein Synthesis, Post-Translational Modification and Degradation: Increase in Expression Levels and Resistance to Sulfhydryl Oxidation of Peroxiredoxin Isoforms in Amyloid β-Resistant Nerve Cells. J. Biol. Chem. 282:30523-30534 (2007). Further, Power, et al. reported that peroxiredoxin 6 was elevated in astrocytes of Alzheimer's disease patients. See, Power, et al., Peroxiredoxin 6 in human brain: Molecular forms, cellular distribution and association with Alzheimer's disease pathology. Acta Neuropathol. 115:610-622 (2008). Another studied reported finding increased levels of nitrated peroxiredoxin 2 in early-onset Alzheimer's disease which they propose hampers the protein's function and contributes to the Alzheimer's phenotype. See, e.g., Reed, et al., Proteomic identification of nitrated brain proteins in early Alzheimer's disease inferior parietal lobule. J. Cell. Mol. Med. 13(8B):2019-2029 (2009). While the exact role of elevated peroxiredoxin in the pathology of Alzheimer's disease is not known, modulation of the function and/or levels of peroxiredoxin family of proteins including, but not limited to, peroxiredoxin 1, peroxiredoxin 2, and peroxiredoxin 6, could have important implications in treating Alzheimer's disease. See, e.g., Cumming, et al., Protein Synthesis, Post-Translational Modification and Degradation: Increase in Expression Levels and Resistance to Sulfhydryl Oxidation of Peroxiredoxin Isoforms in Amyloid β-Resistant Nerve Cells. J. Biol. Chem. 282:30523-30534 (2007).

Other Neurodegenerative Diseases (Parkinson's disease, Amyotrophic Lateral Sclerosis, Down Syndome, Pick's Disease)

In addition to Alzheimer's disease, levels of peroxiredoxin isotypes have been identified as dysregulated and exhibiting elevated expression in Parkinson's disease, Down Syndrome, Pick's disease and amyotrophic lateral sclerosis (ALS; Lou Gherig's disease). See, e.g., Krapfenbauer, et al., Aberrant expression of peroxiredoxin subtypes in neurodegenerative disorders. Brain Res. 967(1-2):152-160 (2003); Basso, et al., Proteome analysis of human substantia nigra in Parkinson's disease. Proteomics 4(12):3943-3952 (2004); Kato, et al., Redox system expression in the motor neurons in amyotrophic lateral sclerosis (ALS): Immunohistochemical studies on sporadic ALS, superoxide dismutase 1 (SOD1)-mutated familial ALS, and SOD1-mutated ALS animal models. Acta Neuropathol. 110(2):101-112 (2005). The post-translational modification of cysteine residues (e.g., by glutathione and nitric oxide) on key proteins important in neurodegenerative processes appears to be important and may impact disease progression (see, e.g., Liedhegner, et al., Mechanisms of Altered Redox Regulation in Neurodegenerative Diseases—Focus on S-Glutathionylation. Antiox. Redox. Signal. 16(6):543-566 (2012); Mieyal, et al., Molecular Mechanisms and Clinical Implications of Reversible Protein S-Glutathionylation. Antiox. Redox. Signal. 10(11):1941-1988 (2008)); therefore, the development of small molecules that can modulate cysteine function is believed to have clinically important potential for the treatment of these diseases.

### Heart Failure (Acute Coronary Syndrome (AC S) and Dilated Cardiomyopathy (DCM))

Elevated levels of thioredoxin (Trx) have been identified in patients with acute coronary syndrome (ACS), dilated cardiomyopathy (DCM), and chronic heart failure (CHF) and while the correlation between this elevation and the heart-related diseases is not clear, Trx levels were positively correlated with the severity of the heart disease. See, e.g., Kishimoto, et al., Serum Thioredoxin (Trx) Levels in Patients with Heart Failure. Jpn. Circ. J. 65:491-494 (2001); Jekell, et al., Elevated circulating levels of thioredoxin and stress in chronic heart failure. Eur. J. Heart Failure 6:883-890 (2004). It is thought, in many cases, that Trx may partially mitigate the damage caused by some heart diseases.

### Hutchinson-Gilford Progeria Syndrome

While protein levels of farnesyltransferase have not been reported in the literature, inhibition of farnesyltransferase has been implicated in the rare disease Hutchinson-Gilford Progeria syndrome (HGPS). Progerin is a key protein that is mutated in progeria and is a truncated variant of prelamin A. Progerin contains a farnesylated cysteine residue at its carboxy-terminus that prevents the protein from dissociating from the nuclear membrane. See, e.g., Capell, et al., Inhibiting farnesylation of progerin prevents the characteristic nuclear blebbing of Hutchinson-Gilford progeria syndrome. Proc. Natl. Acad. Sci. U.S.A. 102(36):12879-12884 (2005); Marji, et al., Defective Lamin A-Rb Signaling in Hutchinson-Gilford Progeria Syndrome and Reversal by Farnesyltransferase Inhibition. PLoS ONE 5(6):311132 (2010) Inhibitors of farnesyltransferase improve the phenotypic symptoms associated with progeria in mouse models, inhibit typical nuclear malformations seen in progeria patients, and restored gene expression in cells from HGPS patients to a normal profile. See, e.g., Capell, et al., Inhibiting farnesylation of progerin prevents the characteristic nuclear blebbing of Hutchinson-Gilford progeria syndrome. Proc. Natl. Acad. Sci. U.S.A. 102(36):12879-12884 (2005); Marji, et al., Defective Lamin A-Rb Signaling in Hutchinson-Gilford Progeria Syndrome and Reversal by Farnesyltransferase Inhibition. PLoS ONE 5(6):311132 (2010).

TABLE 4
Examples of Target Molecule Concentrations/Levels in Selected Tissues

| | | | Examples | |
|---|---|---|---|---|
| | | | of Average | |
| | | Examples of | or Median | |
| | | Ranges of | Protein | |
| | | Protein | Concentration | |
| Target | Disease | Concentration | Values | Reference |
| EGFR | Non-small cell | 0.21-13.58 ng/ | | Dimou A, et |
| | lung cancer | microgram | | al., 2011, |
| | | total protein | | Standardization |
| | | (170 patient | | of Epidermal |
| | | study) | | Growth Factor |
| | | 0.15-89.78 ng/ | | Receptor |
| | | microgram | | (EGFR) |
| | | total protein | | Measurement |
| | | (335 patient | | by Quantitative |
| | | study) | | Immunofluorescence |
| | | | | and |
| | | | | Impact on |
| | | | | Antibody- |
| | | | | Based |
| | | | | Mutation |
| | | | | Detection in |
| | | | | Non-Small |
| | | | | Cell Lung |
| | | | | Cancer, Am J |
| | | | | Path, 179 (2), |
| | | | | 580-589. |
| ALK | Neuroblastoma | | Qualitative | Lamant et al, |
| | | | detection | 2000, |
| | | | only | Expression of |
| | | | (present or | the ALK |
| | | | not | tyrosine kinase |
| | | | present) | gene in |
| | | | ALK was | neuroblastoma, |
| | | | not | Am J Pathol, |
| | | | detected in | 156 (5): 1711-1721. |
| | | | normal or | |
| | | | neoplastic | |
| | | | hematopoietic | |
| | | | tissue | |
| | | | (except for | |
| | | | one t(2; 5)- | |
| | | | positive | |
| | | | ALCL) | |
| | | | ALK was | |
| | | | detected in | |
| | | | neural cell | |
| | | | lines and in | |
| | | | neuroblastomas | |
| ALK | Pediatric | | Qualitatively | Duijkers, et al., |
| | neuroblastoma | | ALK | 2011, |
| | | | levels were | Anaplastic |
| | | | higher in | lymphoma |
| | | | ALK | kinase (ALK) |
| | | | mutant cell | inhibitor |
| | | | lines | response in |
| | | | compared | neuroblastoma |
| | | | to ALK | is highly |
| | | | WT cell | correlated with |
| | | | lines | ALK mutation |
| | | | | status, ALK |
| | | | | mRNA and |
| | | | | protein levels, |
| | | | | Cell Oncol, 34, |
| | | | | 409-417. |
| cMET | | | | http://www.proteinatlas.org/ENSG00000140443/cancer |
| IGF1R | | | High in a | http://www.proteinatlas.org/ENSG00000140443/cancer |
| | | | range of | |
| | | | malignancies | |
| | | | including | |
| | | | breast, | |
| | | | colorectal, | |
| | | | ovarian, | |
| | | | stomach, | |
| | | | liver, and | |
| | | | pancreatic. | |
| | | | Widely | |
| | | | expressed | |
| | | | in both | |
| | | | cancer and | |
| | | | normal | |
| | | | tissue. | |
| ROS1 | | | Not widely | http://www.proteinatlas.org/ENSG00000140443/cancer |
| | | | expressed | |
| | | | in normal | |
| | | | tissue, | |
| | | | found | |
| | | | predominantly | |
| | | | in | |
| | | | kidney, | |
| | | | brain, | |
| | | | esophagus, | |
| | | | heart, and | |
| | | | some soft | |
| | | | tissues. | |
| | | | Moderately | |
| | | | expressed | |
| | | | in | |
| | | | colorectal, | |
| | | | pancreatic, | |
| | | | and skin | |
| | | | cancer and | |
| | | | melanoma. | |
| | | | Also | |
| | | | expressed | |
| | | | in head and | |
| | | | neck | |
| | | | cancer, | |
| | | | liver, lung, | |
| | | | renal and | |
| | | | stomach | |
| | | | cancer. | |
| Trx | Acute lung | BAL normal | Bronchioal | Callister et al, |
| | injury/Acute | 1-49 ng/mL | veolar | 2005, |
| | Respiratory | ALL/ARDS | lavage | Extracellular |
| | Distress | (1-500 ng/mL | levels 16.6 ng/mL | thioredoxin |
| | Syndome | Plasma normal | (normal) | levels are |
| | | 5-48 ng/mL | vs 61.6 ng/mL | increased in |
| | | ALL/ARDS | (ALL/ARDS) | patients with |
| | | 12.5->125 ng/mL | Plasma | acute lung |
| | | (ranges | levels 18.0 ng/mL | injury, Thorax, |
| | | deduced from | (normal) vs | 61: 521-527. |
| | | graphs) | 36.2 ng/mL | |
| | | | (ALL/ARDS) | |
| Trx | Healthy | Healthy | | Powis G, |
| | volunteers | volunteers: | | Montfort WR, |
| | | 10-80 ng/mL | | 2001, |
| | | in plasma with | | Properties and |
| | | values | | biological |
| | | typically less | | activities of |
| | | than 30 ng/mL | | thioredoxins, |
| | | | | Annu Rev |
| | | | | Pharmacol |
| | | | | Toxicol, 41, |
| | | | | 261-295. |
| Trx | Healthy | | Healthy | Baker et al, |
| | Volunteers and | | volunteers: | 2006, The |
| | Solid Tumor | | 27.6 +/− | antitumor |
| | Cancer Patients | | 10.8 ng/mL | thioredoxin-1 |
| | Solid tumors | | in plasma | inhibitor PX- |
| | included: | | Solid | 12 (1- |
| | colorectal, | | Tumor | methylpropyl |
| | NSCLC, lung | | Volunteers: | 2-imidazolyl |
| | adenoma, | | 182.0 +/− | disulfide) |
| | hepatocellular | | 21.8 ng/mL | decreases |
| | carcinoma, | | in plasma | thioredoxin-1 |
| | cholangiocarcinoma, | | | and VEGF |
| | sarcoma, | | | levels in cancer |
| | and pancreatic | | | patient plasma, |
| | cancer | | | J Lab Clin |
| | | | | Med, 147 (2) |
| | | | | 83-90. |
| Trx | Heart Failure | | Dilated | Kishimoto et |
| | | | cardiomyopathy | al, 2001, |
| | | | 36.9 ± | Serum |
| | | | 8.6 ng/mL | Thioredoxin |
| | | | Acute | (Trx) Levels in |
| | | | coronary | Patients with |
| | | | syndrome | Heart Failure, |
| | | | 30.6 ± 4.9 ng/mL | Jpn Circ J, |
| | | | Control | 65: 491-494. |
| | | | patients | |
| | | | 14.0 ± 4.6 ng/mL | |
| Glutaredoxin | Healthy | | Healthy | Nakamura et |
| | volunteers and | | volunteers: | al, |
| | Heart Surgery | | 456 ± 284 ng/mL | Measurements |
| | Patients | | in | of plasma |
| | | | plasm | glutaredoxin |
| | | | Heart | and |
| | | | patients: | thioredoxin in |
| | | | similar to | healthy |
| | | | healthy | volunteers and |
| | | | volunteers | during open- |
| | | | | heart surgery |
| Peroxiredoxin 1 | Ovarian cancer | In proximal | Ovarian | Hoskins et al, |
| | patient | fluids (ascites, | cancer | 2011, |
| | | tissue | patients: | Proteomic |
| | | interstitial | 26.0 ng/mL ± | analysis of |
| | | fluid, etc) | 9.27 in | ovarian cancer |
| | | Ovarian cancer | proximal | proximal |
| | | patients: 0-150 ng/mL | fluids | fluids: |
| | | Control | (ascites, | validation of |
| | | patients | tissue | elevated |
| | | (benign | interstitial | peroxiredoxin |
| | | ovarian | fluid, etc) | 1 in patient |
| | | pathology) 0-50 ng/mL | Patients | peripheral |
| | | | with | circulation |
| | | | benign | |
| | | | ovarian | |
| | | | pathology: | |
| | | | 4.19 ± 2.58 | |
| ERCC1 | Epithelial | | Expression | DeLoia et al, |
| | ovarian cancer | | is highly | 2012, |
| | | | variable | Comparison of |
| | | | across | ERCC1/XPF |
| | | | EOC | genetic |
| | | | patients | variation, |
| | | | and | mRNA and |
| | | | quantitation | protein levels |
| | | | is relative | in women with |
| | | | to β-actin | advanced stage |
| | | | controls | ovarian cancer |
| | | | with no | treated with |
| | | | defined | intraperitoneal |
| | | | units | platinum, |
| | | | | Gynecol Oncl, |
| | | | | 126 (3), 448-454. |
| RNR | | | | |
| Tubulin | Mouse | | Tubulin is | Gard DL and |
| | Neuroblastoma | | highly | Kirschner |
| | cells | | abundant | MW, 1997, |
| | | | constituting | Microtubule |
| | | | 2% or more | Assembly in |
| | | | of total | Cytosplasmic |
| | | | protein in | Extracts of |
| | | | cells with | Xenopus |
| | | | similar | Oocytes and |
| | | | distribution | Eggs, J. Cell |
| | | | across | Biol., 105, |
| | | | species | 2191, 2201. |
| | | | Up to 2.4 mg/mL | Olmsted JB, |
| | | | in | 1981, Tubulin |
| | | | egg and | Pools in |
| | | | oocyte | Differentiating |
| | | | extracts | Neuroblastoma |
| | | | which is 24 | Cells, J. Cell |
| | | | micromolar | Biol., 89, 418-423. |
| | | | 1.6 mg/mL | |
| | | | 4 pg/cell | |
| Farnesyl- | | | | http://www.proteinatlas.org/ENSG00000140443/cancer |
| transferase | | | | |

TABLE 5
Examples of mRNA Levels in Selected Biological Samples (results do not have
units and are relative to controls indicated; for all targets additional relative
levels can be found at www.proteinatlas.org).

| | | Examples of Expression | |
|---|---|---|---|
| Target | Disease | Levels | Reference |
| EGFR | Metastatic | 7% (approximately 4 of 51) | Santarpia et al., 2011, mRNA expression |
| | Non-small | of patients expressed EGFR | levels and genetic status of genes involved in |
| | Cell Lung | del 19 | the EGFR and NF-κB pathways in metastatic |
| | Cancer | 2% (approximately 1 of 51) | non-small-cell lung cancer patients, J |
| | | of patients expressed EGFR | Translational Med, 9, 163 (1-9). |
| | | L858R | |
| EGFR | Lung cancer | Gene copy numbers for | Kanteti et al., 2009, MET HGF, EGFR and |
| | | EGFR ranged between 3-50 | PXN gene copy number in lung cancer using |
| | | in a range of NSCLC cell | DNA extracts from FFPE archival samples, |
| | | line. | and prognostic significance, J. Environ |
| | | | Pathol Toxicol Oncol, 28 (2), 89-98. |
| ALK | Neuroblastoma | Mutated ALK RNA levels | Schulte et al., 2011, High ALK receptor |
| | | were 2 fold elevated relative | tyrosine kinase expression supersedes ALK |
| | | to WT ALK RNA levels | mutation as a determining factor of an |
| | | | unfavorable phenotype in primary |
| | | | neuroblastoma, Clin Cancer Res, 17 (15), |
| | | | 5082-5092. |
| ALK | Neuroblastoma | ALK was not detected in | Lamant et al, 2000, Expression of the ALK |
| | | normal or neoplastic | tyrosine kinase gene in neuroblastoma, Am J |
| | | hematopoietic tissue (except | Pathol, 156 (5): 1711-1721. |
| | | for one t(2; 5)-positive ALCL. | |
| | | ALK was detected in neural | |
| | | cell lines and in | |
| | | neuroblastomas | |
| ALK | Anaplastic | | |
| | large-cell | | |
| | lymphoma | | |
| MET | Non-small cell | MET was 2-10 fold increased | Olivero et al., 1996, Overexpression and |
| | lung cancer | in non-small cell lung cancer | activation of hepatocyte growth factor/scatter |
| | | samples compared to normal | factor in human non-small-cell lung |
| | | tissues | carcinomas, Br j Cancer, 74: 1862-1868. |
| MET | Small cell lung | MET receptor was highly | Ma et al, 2003, c-MET Mutational Analysis |
| | cancer | expressed in 6 of 10 small | in Small Cell Lung Cancer: Novel |
| | | cell lung cancer cell lines; | Juxtamembrane Domain Mutations |
| | | however, level of expression | Regulating Cytoskeletal Functions, 63, 6272-6281. |
| | | did not correspond to | |
| | | receptor mutation. | |
| | | Mutations detected in small | |
| | | cell lung cancer cell lines and | |
| | | patient tissues | |
| IGF1R | | | |
| ROS1 | | | |
| Trx | | | |
| Grx | | | |
| Prx | | | |
| ERCC1 | | | |
| RNR | | | |
| Tubulin | | | |
| Farnesyltranferase | Primary Liver | 87.5% overexpression | Sui et al., 2012 Expression of |
| | Cancer | relative to normal liver tissue | farnesyltransferase in primary liver cancer, |
| | | | Chin Med J, 125 (14) 2427-2431. |

### SUMMARY OF THE INVENTION

The invention described and claimed herein has many attributes and embodiments including, but not limited to, those set forth or described or referenced in this Summary section. However, it should be noted that this Summary is not intended to be all-inclusive, nor is the invention described and claimed herein limited to, or by, the features, embodiments, or definitions identified in said Summary. Moreover, this Summary is included for purposes of illustration only, and not restriction.

Large numbers of current approaches to the treatment of cancer and many other diseases have been focused on identifying a single genetic or molecular target of interest, and then developing therapies to interact with the identified target in order to treat the disease. An example of this focus is the growing trend in oncology to seek “personalized therapies” aimed at addressing a particular genetic mutation in an identified portion of the cancer population.

While many of these approaches can provide some benefit to patients, they are only a first step towards achieving comprehensive and lasting treatment benefits. This is due to the heterogeneous nature of cancer and many other diseases. Because cancer is heterogeneous, single-targeted approaches frequently leave other cancer-implicated targets and pathways unaddressed, allowing the underlying disease to progress.

Given the limitations of these current approaches, a treatment that was able to contemporaneously interact with multiple targets of interest would be beneficial and would represent a next step in treating cancer and many other diseases.

The teachings in the present application take into account the concept of disease heterogeneity, in combination with new observations and data, in order to provide novel pharmaceutical compositions, methods, and kits used for the treatment of cancer and other medical conditions.

Unlike the current trend to seek single-targeted approaches, the present invention teaches compositions and methods to contemporaneously modulate and interact with multiple targets of interest in order to provide treatment for a variety of cellular metabolic anomalies or other undesirable physiological conditions.

One embodiment of the present invention discloses a contemporaneous, heterogeneously-oriented, multi-targeted method comprising the therapeutic modification and/or modulation of one or more types of disease (including cancer) for purposes of minimizing or overcoming the deleterious physiological ramifications of, e.g., cancer heterogeneity, where the method is comprised of the modification and/or modulation of:

(i) the expression level and/or (ii) the biochemical function of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; by the administration of the sulfur-containing, amino acid-specific small molecules of the present invention in an amount sufficient to provide a therapeutic benefit to a subject having one or more types of cancer where the expression level and/or biochemical function of one or more target molecule is abnormal and metabolic modification and/or modulation of the target molecule(s) is used to treat the subject in need thereof.

Another embodiment of the present invention discloses a contemporaneous, heterogeneously-oriented, multi-targeted method comprising the therapeutic modification and/or modulation of one or more types of disease (including cancer) for purposes of minimizing or overcoming the deleterious physiological ramifications of, e.g., cancer heterogeneity, where the method is comprised of the modification and/or modulation of: (i) the expression level and/or (ii) the biochemical function of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; by the administration of the sulfur-containing, amino acid-specific small molecules of the present invention in an amount sufficient to provide a therapeutic benefit to a subject with one or more types of cellular metabolic anomalies or other undesirable physiological conditions where the expression level and/or biochemical function of one or more target molecule is abnormal and metabolic modification and/or modulation of the target molecule(s) is used to treat the subject in need thereof.

One embodiment of the present invention discloses a method for the metabolic modification and/or modulation of the expression level of multiple target molecules; where the target molecules are selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif; and where the method is comprised of the administration of the sulfur-containing, amino acid-specific small molecules of the present invention in an amount sufficient to provide a therapeutic benefit to a subject suffering from one or more types of cellular metabolic anomalies or other pathophysiological conditions where the expression levels of one or more of the target molecules is abnormally elevated and metabolic modification and/or modulation of the target molecule(s) is used to treat the cellular metabolic anomalies or other pathophysiological conditions.

Another embodiment of the present invention discloses a method for the metabolic modification and/or modulation of the biochemical activity of multiple target molecules; where the target molecules are selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif; and where the method is comprised of the administration of the sulfur-containing, amino acid-specific small molecules of the present invention in an amount sufficient to provide a therapeutic benefit to a subject suffering from one or more types of cellular metabolic anomalies or other pathophysiological conditions where the biochemical activities of the multiple target molecules are abnormal and cellular metabolic modification and/or modulation is used to treat said cellular metabolic anomalies or other pathophysiological conditions.

In various embodiments of the present invention the sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu; and

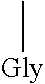

pharmaceutically-acceptable salts thereof.

In various embodiments of the present invention, cancers selected from the group consisting of: lung cancer, colorectal cancer, gastric cancer, esophageal cancer, cancer of the biliary tract, gallbladder cancer, breast cancer, cervical cancer, ovarian cancer, endometrial cancer, vaginal cancer, myeloma, uterine cancer, prostate cancer, hepatic cancer, adenocarcinoma, pancreatic cancer, brain cancer, and various types of skin cancer, including melanoma, are disclosed.

In yet other embodiments of the present invention, the cellular metabolic anomalies or other pathophysiological conditions are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigmentosum, Cockayne syndrome, Fanconi anemia, and cerebro-oculo-facio-skeletal syndrome.

One embodiment of the present invention discloses a method to modify and/or modulate the intracellular environment of cancer cells in a subject suffering from cancer such that the intracellular environment of said cancer cells is made more amenable to the pharmacological activity of cancer treating agent(s) administered to treat the subject's cancer; where the method is comprised of the administration of an amount of the sulfur-containing, amino acid-specific small molecules of the present invention sufficient to modify and/or modulate the intracellular environment of cancer cells in the subject suffering from cancer; and where the cancer involves: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif.

Another embodiment of the present invention discloses a method to modify and/or modulate the intracellular environment of cells in a subject suffering from cellular metabolic anomalies or other pathophysiological conditions such that the intracellular environment of the cells is made more amenable to the pharmacological activity of medicinal agent(s) administered to treat the subject's cellular metabolic anomalies or other pathophysiological conditions; where the method is comprised of the administration of an amount of the sulfur-containing, amino acid-specific small molecules of the present invention sufficient to modify and/or modulate the intracellular environment of cells in the subject suffering from the cellular metabolic anomalies or other pathophysiological conditions where the cellular metabolic anomalies or other pathophysiological conditions involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif.

In various embodiments of the present invention, cancers selected from the group consisting of: colorectal cancer, gastric cancer, esophageal cancer, cancer of the biliary tract, gallbladder cancer, breast cancer, cervical cancer, ovarian cancer, endometrial cancer, lung cancer, vaginal cancer, uterine cancer, prostate cancer, hepatic cancer, adenocarcinoma, pancreatic cancer, brain cancer, lung cancer, various types of skin cancer (e.g., melanoma), and myeloma, lymphoma and other cancers of the blood are disclosed.

In various embodiments of the present invention, cellular metabolic anomalies or other pathophysiological conditions of non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigmentosum, Cockayne syndrome, Fanconi anemia, and cerebro-oculo-facio-skeletal syndrome are disclosed.

One embodiment of the present invention discloses a method for treating a subject suffering from cancer where a multi-targeted, molecular-directed treatment regimen is beneficial in overcoming cellular metabolic resistance to treatment in a subject with cancer that is heterogeneous; where the cellular metabolic resistance to treatment is associated with: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif; and where the method is comprised of the administration of an amount of the sulfur-containing, amino acid-specific small molecules of the present invention sufficient to overcome the cellular metabolic resistance to treatment in said subject with cancer that is heterogeneous.

Another embodiment of the present invention discloses a method for treating a subject suffering from cellular metabolic anomalies or other pathophysiological conditions where a heterogeneous, multiple targeted, molecular-directed treatment regimen is beneficial in overcoming cellular metabolic resistance to treatment in the subject with cellular metabolic anomalies or other pathophysiological conditions; wherein the cellular metabolic resistance to treatment is associated with: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif; and where the method is comprised of the administration of an amount of the sulfur-containing, amino acid-specific small molecules of the present invention sufficient to overcome the cellular metabolic resistance to treatment in the subject with cellular metabolic anomalies or other pathophysiological conditions.

In one embodiment of the present invention a method to determine the amount of the sulfur-containing, amino acid-specific small molecules of the present invention required to be administered to provide a therapeutic benefit to a subject with cancer that involves: (i) the abnormal biochemical activity and/or (ii) the abnormal expression of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif; where the method is comprised of determining: (i) the abnormal biochemical activity and/or (ii) the abnormal expression of the target molecules and then using the results obtained to select the amount of the sulfur-containing, amino acid-specific small molecules of the present invention to administer to provide a therapeutic benefit to the subject in need thereof is disclosed. The method of determining the amount of the sulfur-containing, amino acid-specific small molecules of the present invention required to be administered to provide a therapeutic benefit to a subject with cancer that involves: (i) the abnormal biochemical activity and/or (ii) the abnormal expression of target molecules is selected from the group consisting of: (i) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (ii) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (iii) PCR coupled with MS approaches including, but not limited to, MALDI-TOF MS (e.g., Sequenom's MassARRAY system and related approaches); (iv) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (v) isoelectric focusing, agarose/polyacrylamide gel electrophoresis, Southern blotting, Western blotting, Northern blotting, enzyme/substrate activity assay, X-ray crystallography, and other related analytic methodologies.

In another embodiment of the present invention, a method to determine the amount of the sulfur-containing, amino acid-specific small molecules of the present invention required to be administered to provide a therapeutic benefit to a subject with cellular metabolic anomalies or other undesirable physiological conditions that involve: (i) the abnormal biochemical activity and/or (ii) the abnormal expression of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif; where the method is comprised of determining (i) the abnormal biochemical activity and/or (ii) the abnormal expression of the target molecules and then using the results obtained to select the amount of the sulfur-containing, amino acid-specific small molecules of the present invention to administer to provide a therapeutic benefit to the subject in need thereof is disclosed.

In one embodiment of the present invention a method for use in: (a) the selection of subjects for treatment; (b) the determination of the most effective chemotherapeutic agent(s) to be administered in combination with the administration of the sulfur-containing, amino acid-specific small molecules of the present invention; (c) the dosage of the chemotherapeutic agent(s) to be administered; (d) the determination of the length and/or number of treatment cycles; and/or (e) adjustment of the specific chemotherapeutic agent(s) used and the dosage administered during treatment of a subject having cancer is disclosed; where the method is comprised of quantitatively determining the levels of expression of target molecules selected from the group consisting of: and other target molecules possessing a similar active site or structural motif, and then using these expression levels in determining: (i) the specific subjects to be treated; (ii) the chemotherapeutic agent(s) to be administered in combination with the administration of the sulfur-containing, amino acid-specific small molecules of the present invention; (iii) the dosage of the chemotherapeutic agent(s) to be administered; (iv) the length and/or number of chemotherapeutic treatment cycles to be administered; and/or (v) the adjustment of the specific chemotherapeutic agent(s) used and the dosages administered during the treatment regimen of the subject having cancer.

In various embodiments of the present invention, cancers selected from the group consisting of: colorectal cancer, gastric cancer, esophageal cancer, cancer of the biliary tract, gallbladder cancer, breast cancer, cervical cancer, ovarian cancer, endometrial cancer, vaginal cancer, uterine cancer, prostate cancer, hepatic cancer, adenocarcinoma, pancreatic cancer, brain cancer, lung cancer, various types of skin cancer (e.g., melanoma), and lymphoma and other cancers of the blood are disclosed.

In various embodiments of the present invention, cancer treating agent(s) are selected from the groups consisting of: (i) fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; aziridine-containing compounds, and various other cytotoxic and cytostatic agents; (ii) hormones, hormonal complexes, and antihormones selected from the group comprising: interleukins, interferons, leuprolide, and pegasparaginase; (iii) enzymes, proteins, peptides, and antivirals, including enzymes, proteins, peptides, and antivirals selected from the group consisting of: acyclovir and zidovudine; (iv) cytotoxic agents and cytostatic agents; (v) polyclonal and monoclonal antibodies, including agents selected from the group consisting of: crizotinib, gefitinib, erlotinib, cetuximab, afatinib, dacomitinib, ramucirumab, necitumumab, lenvatinib, palbociclib, alectinib, zybrestat, tecemotide, obinutuzumab (GA101), AZD9291, CO-1686, vintafolide, CRLX101, ipilimumab, yervoy, nivolumab, ibrutinib, selumetinib, olaparib, trastuzumab, lucitanib, rucaparib, NOV-002, MPDL3280A, pembrolizumamb, lambrolizumab (MK-3475), MEDI4736, tremelimumab, AMP-514, MEDI6469, RG7446, CRS-207, GVAX, ceritinib (LDK378), IMCgp100, vemurafenib (Zelboraf), cabozantinib, CTL019, LEE011, T-DM1, MM-121, bavituximab, MAGE-A3, axitinib, ipilimumab, rituximab, tivantinib, and the like; (vi) PD-1 checkpoint receptor inhibiting agents, PD-L1 checkpoint receptor inhibiting agents, and other checkpoint receptor inhibiting agents; (vii) immune checkpoint pathway modulatory antibodies; (viii) kinase inhibitors; (ix) ALK inhibitors; (ix) cancer vaccines; (x) Antibody Drug Conjugates; and/or (xi) chimeric antigen receptor T-cell (CAR-T) Therapy.

In various embodiments of the present invention, the subjects selected for treatment are further categorized into various subtypes for more beneficial treatment which include, but are not limited to, female subjects; non-smoker subjects; female, non-smoker subjects; male, non-smoker subjects; non-smoker subjects with expression of ALK and/or MET; subjects over 65 years of age; subjects whose cancer has been categorized as Stage M1a or M1b; subjects who are currently being or have previously been treated with paclitaxel and/or cisplatin, and various combinations of the foregoing.

In another embodiment of the present invention, a method for use in: (a) the selection of specific subjects for treatment; (b) the determination of the most effective medicinal agent(s) in combination with the administration of the sulfur-containing, amino acid-specific small molecules of the present invention; (c) the dosage of the medicinal agent(s) to be administered; (d) the determination of the length and/or number of treatment cycles to be administered; and/or (e) adjustment of the specific medicinal agent(s) used and the dosages administered during treatment of a subject with non-cancerous, cellular metabolic anomalies or other pathophysiological conditions is disclosed; where the method is comprised of quantitatively determining the levels of expression of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif, and then using these expression levels in determining: (i) the specific subjects to be treated; (ii) the medicinal agent(s) to be administered in combination with the administration of the sulfur-containing, amino acid-specific small molecules of the present invention; (iii) the dosage of the medicinal agent(s) to be administered; (iv) the length and/or number of treatment cycles to be administered; and/or (v) the adjustment of the specific medicinal agent(s) administered and the dosages administered during treatment regimen of the subject having cellular metabolic anomalies or other pathophysiological conditions.

In yet another embodiment of the present invention, a method is disclosed for maximizing the length of time before there is cancer progression in a subject who has cancer that involves: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and other target molecules possessing a similar active site or structural motif; where the method comprises the administration of the sulfur-containing, amino acid-specific small molecules of the present invention which function to delay the reoccurrence and/or progression of the cancer in the subject by modifying and/or modulating: (i) the abnormal biochemical activity and/or (ii) the abnormal expression of any combination of the target molecules.

Another embodiment of the present invention discloses a kit for use in the treatment of a subject having cancer that is resistant to the chemotherapeutic agent(s) being used to treat the subject with cancer, where the cancer is any cancer which: (i) abnormally overexpresses anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase (RNR), tubulin, farnesyltransferase, and/or other target protein (possessing a similar active site or structural motif) and/or (ii) exhibits evidence of anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, and/or other target protein (possessing a similar active site or structural motif)-mediated resistance to the chemotherapeutic agent(s) being used to treat the subject with cancer; where the said kit comprises: (a) one or more chemotherapy agents; (b) the sulfur-containing, amino acid-specific small molecules of the present invention; and (c) instructions for administering said chemotherapy agent(s) and the sulfur-containing, amino acid-specific small molecules of the present invention to a subject with types of cancer that are resistant to the chemotherapeutic agent(s) being used to treat the subject with cancer.

A further embodiment of the present invention discloses a kit for use in the treatment of a subject having cancer that is resistant to the chemotherapeutic agent(s) being used to treat the subject with cancer, where the cancer is any cancer which: (i) possesses abnormal biochemical activity in anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, and/or other target protein (possessing a similar active site or structural motif) and/or (ii) exhibits evidence of anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and/or other target protein (possessing a similar active site or structural motif)-mediated resistance to the chemotherapeutic agent(s) being used to treat the subject with cancer; where the kit comprises: (a) one or more chemotherapy agents; (b) the sulfur-containing, amino acid-specific small molecules of the present invention; and (c) instructions for administering said chemotherapy agent(s) and the sulfur-containing, amino acid-specific small molecules of the present invention to a subject with cancer which is resistant to the chemotherapeutic agent(s) being used to treat the subject with cancer.

In one embodiment of the present invention is disclosed a kit comprising: (a) one or more medicinal agents; (b) the sulfur-containing, amino acid-specific small molecules of the present invention; and (c) instructions for the administration of said medicinal agents and the sulfur-containing, amino acid-specific small molecules of the present invention to a subject having cellular metabolic anomalies or other undesirable physiological conditions that cause: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; where the sulfur-containing, amino acid-specific small molecules of the present invention function to modify and/or modulate the abnormal biochemical activity and/or abnormal expression of the target molecules in the subject having cellular metabolic anomalies or other undesirable physiological conditions.

In another embodiment of the present invention is disclosed a kit comprising: (a) one or more chemotherapy agents; (b) the sulfur-containing, amino acid-specific small molecules of the present invention; and (c) instructions for administering said chemotherapy agent(s) and the sulfur-containing, amino acid-specific small molecules of the present invention to a subject with a type of cancer that is generally less responsive to particular types of chemotherapeutic treatments; where the sulfur-containing, amino acid-specific small molecules of the present invention are administered in an amount sufficient to cause an increase in the cytotoxic or cytostatic activity of the administered chemotherapeutic agent(s) whose cytotoxic or cytostatic activity was heretofore adversely affected by: (i) the abnormal biochemical activity and/or (ii) the abnormal expression of the target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; where the sulfur-containing, amino acid-specific small molecules of the present invention function to modify and/or modulate the abnormal biochemical activity and/or the abnormal expression of the target molecules.

One embodiment of the present invention discloses a medicament which modifies and/or modulates the expression levels of the target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; where the medicament is the sulfur-containing, amino acid-specific small molecules of the present invention administered in an amount sufficient to provide a therapeutic benefit to a subject having a type of cellular metabolic anomaly or other undesirable physiological condition where the expression levels of said target molecules are abnormally elevated and must be modified and/or modulated in order to treat said cellular metabolic anomaly or other undesirable physiological condition.

A further embodiment of the present invention discloses a medicament which modifies and/or modulates the biochemical activity of the target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; wherein said medicament is the sulfur-containing, amino acid-specific small molecules of the present invention administered in an amount sufficient to provide a therapeutic benefit to a subject having a cellular metabolic anomaly or other undesirable physiological condition where the biochemical activities of the target molecules are abnormal and must be modified and/or modulated in order to treat the cellular metabolic anomaly or other undesirable physiological condition.

In another embodiment of the present invention is disclosed a method for the prophylactic use of the sulfur-containing, amino acid-specific small molecules of the present invention administered in an amount sufficient to provide a prophylactic benefit to a subject who has previously suffered from a form of cancer that involves: (i) the abnormal biochemical activity and/or (ii) the abnormal expression of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; where the sulfur-containing, amino acid-specific small molecules of the present invention function to prevent the reoccurrence of the cancer in the subject by modifying and/or modulating: (i) the abnormal biochemical activity and/or (ii) the abnormal expression of the target molecules.

In a further embodiment of the present invention is disclosed a method for the prophylactic use of the sulfur-containing, amino acid-specific small molecules of the present invention administered in an amount sufficient to provide a prophylactic benefit to a subject who has previously suffered from a type of cellular metabolic anomaly or other undesirable physiological condition that involves: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; where the sulfur-containing, amino acid-specific small molecules of the present invention function to prevent the reoccurrence of the cellular metabolic anomaly or other undesirable physiological condition in the subject by modifying and/or modulating: (i) the abnormal biochemical activity and/or (ii) the abnormal expression of any combination of the target molecules.

A further embodiment of the present invention discloses a method to restore normal cellular biochemical function and/or normal expression levels of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; where the method is comprised of the administration of the sulfur-containing, amino acid-specific small molecules of the present invention in an amount sufficient to provide a therapeutic benefit to a subject having cancer where the normal cellular biochemical function and/or the expression levels of the target molecules are abnormal and must be modified and/or modulated in order to treat the subject with cancer.

One embodiment of the present invention discloses a method to restore the normal cellular biochemical function and/or the expression level of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; where the method is comprised of the administration of the sulfur-containing, amino acid-specific small molecules of the present invention in an amount sufficient to provide a therapeutic benefit to a subject having a cellular metabolic anomaly or other undesirable physiological condition, including cancer, where the normal cellular biochemical function and/or the expression levels of the target molecules are abnormal and must be modified and/or modulated in order to treat the subject with a metabolic anomaly or other undesirable physiological condition, including cancer.

Another embodiment of the present invention discloses a method for the maintenance of a subject having cancer; where the method is comprised of the modification and/or modulation of: (i) the expression level and/or (ii) the biochemical function of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; and where the method is comprised of the administration of the sulfur-containing, amino acid-specific small molecules of the present invention in an amount sufficient to provide a therapeutic benefit to a subject having a type of cancer where the expression level and/or biochemical function of one or more target molecule is abnormal and metabolic modification and/or modulation of the target molecule(s) is used to treat the subject in need thereof.

Another embodiment of the present invention discloses a treatment method which comprises the administration of one or more cancer treating agents and an amount of the sulfur-containing, amino acid-specific small molecules of the present invention sufficient to provide a therapeutic benefit to a subject with lymphoma, acute lymphocytic leukemia (ALL), or acute myelogenous leukemia (AML) cancers that involve: (i) the abnormal biochemical activity and/or (ii) the abnormal expression of the tyrosine kinase enzyme, anaplastic lymphoma kinase (ALK) or epidermal growth factor receptor (EGFR).

In a further embodiment of the present invention is disclosed a method for the formation of adducts comprising the covalent-binding of one or more sulfur-containing, amino acid-specific small molecules of the present invention to cysteine amino acid residues within a target molecule selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif where the adduct formation has the ability to modify and/or modulate abnormal expression and/or biochemical activity of said target molecule(s) so as to provide a therapeutic benefit to a subject with one or more types of cellular metabolic anomalies or other undesirable physiological conditions that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif.

Another embodiment discloses a method for quantitatively ascertaining the level of DNA, mRNA, and/or protein of a target molecule selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif or in other target molecules possessing cysteine residues with similar functional or structural characteristics, in cells which have been isolated from a patient who is suspected of having cancer or has already been diagnosed with cancer; where the method used to identify levels of the DNA, mRNA, and/or protein of a target molecule(s) is selected from the group consisting of: (i) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (ii) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (iii) PCR coupled with MS approaches including, but not limited to, MALDI-TOF MS (e.g., Sequenom's MassARRAY system and related approaches); (iv) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (v) isoelectric focusing, agarose/polyacrylamide gel electrophoresis, Southern blotting, Western blotting, Northern blotting, enzyme/substrate activity assay, X-ray crystallography, and other related analytic methodologies.

Normal levels of the protein targets described above have been determined in a range of biological samples (cell lines, biological fluids, patient samples, and the like) and some examples are summarized in Table 4. Additionally, the amount of up- or down-regulation of the RNAs corresponding to the protein targets described above has been determined and some examples are summarized in Table 5. Additionally, the Human Protein Atlas (see, www.proteinatlas.org) provides information on mRNA and protein expression patterns for all of the targets cited herein across a range of biological samples although the data on this site is not exhaustive. Further, in addition to statistically significant changes in protein levels or mRNA levels, sometimes it is the mere expression of a mutated gene or the production of a mutated, fused, or truncated protein that results in or shows evidence of the disease phenotype. In these cases, the changes in the overall “levels” of mRNA or protein may be quite low but they nevertheless have significant biological effects.

A further embodiment discloses a method for quantitatively ascertaining the level of DNA, mRNA, and/or protein of a target molecule for the purpose of providing treatment with the sulfur-containing, amino acid-specific small molecules of the present invention, where the target molecule is selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif or in other target molecules possessing cysteine residues with similar functional or structural characteristics, in cells which have been isolated from a patient who is suspected of having a non-cancerous cellular metabolic anomaly or other undesirable physiological condition or has already been diagnosed with a non-cancerous cellular metabolic anomaly or other undesirable physiological condition; where the method used to identify levels of the DNA, mRNA, and/or protein of a target molecule(s) is selected from the group consisting of: (i) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (ii) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (iii) PCR coupled with MS approaches including, but not limited to, MALDI-TOF MS (e.g., Sequenom's MassARRAY system and related approaches); (iv) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (v) isoelectric focusing, agarose/polyacrylamide gel electrophoresis, Southern blotting, Western blotting, Northern blotting, enzyme/substrate activity assay, X-ray crystallography, and other related analytic methodologies.

A further embodiment of the present invention discloses a method for improving biological system stability in a subject with one or more types of cancer, where the system stability is impacted by: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; and where the method is comprised of the administration of the sulfur-containing, amino acid-specific small molecules of the present invention in an amount sufficient to provide a therapeutic benefit by improving biological system stability in the subject with one or more types of cancer.

Another embodiment of the present invention discloses a method for improving biological system stability in a subject having one or more types of cellular metabolic anomalies or other pathophysiological conditions, where the system stability is impacted by: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; and where the method is comprised of the administration of the sulfur-containing, amino acid-specific small molecules of the present invention in an amount sufficient to provide a therapeutic benefit by improving biological system stability in the subject having one or more types of cellular metabolic anomalies or other pathophysiological conditions.

A further embodiment of the present invention discloses a method for improving biological system stability by altering the relative level of non-clonal chromosomal aberrations (NCCAs) in a subject with one or more types of cancer, where the relative level of non-clonal chromosomal aberrations (NCCAs) is impacted by: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; and where the method is comprised of the administration of the sulfur-containing, amino acid-specific small molecules of the present invention in an amount sufficient to provide a therapeutic benefit by altering the relative level of non-clonal chromosomal aberrations (NCCAs) in the subject with one or more types of cancer.

Yet another embodiment of the present invention discloses a method for improving biological system stability by altering the level of non-clonal chromosomal aberrations (NCCAs) in a subject having one or more types of cellular metabolic anomalies or other pathophysiological conditions, where the level of non-clonal chromosomal aberrations (NCCAs) is impacted by: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; and where the method is comprised of the administration of the sulfur-containing, amino acid-specific small molecules of the present invention in an amount sufficient to provide a therapeutic benefit by altering the relative level of non-clonal chromosomal aberrations (NCCAs) in the subject having one or more types of cellular metabolic anomalies or other pathophysiological conditions.

One embodiment of the present invention discloses a method for improving biological system stability by altering the elevated levels of non-clonal chromosomal aberrations (NCCAs) in a subject having one or more types of cancer, where the method is comprised of the administration of the sulfur-containing, amino acid-specific small molecules of the present invention in an amount sufficient to provide a therapeutic benefit by modifying and/or modulating the elevated level of non-clonal chromosomal aberrations (NCCAs) in the subject having one or more types of cancer.

Another embodiment of the present invention discloses a method for improving biological system stability by altering the elevated levels of non-clonal chromosomal aberrations (NCCAs) in a subject having one or more types of non-cancerous cellular metabolic anomalies or other pathophysiological conditions, where the method is comprised of the administration of the sulfur-containing, amino acid-specific small molecules of the present invention in an amount sufficient to provide a therapeutic benefit by modifying and/or modulating the elevated level of non-clonal chromosomal aberrations (NCCAs) in the subject having one or more types of non-cancerous cellular metabolic anomalies or other pathophysiological conditions.

In a further embodiment of the present invention is disclosed a method for the prophylactic use of the sulfur-containing, amino acid-specific small molecules of the present invention administered in an amount sufficient to provide a prophylactic benefit to a subject who has previously suffered from a type of cellular metabolic anomaly or other undesirable physiological condition that involves: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif; and where the method is comprised of the administration of the sulfur-containing, amino acid-specific small molecules of the present invention in an amount sufficient to provide a therapeutic benefit to a subject suffering from one or more types of cellular metabolic anomalies or other pathophysiological conditions where the biochemical activities of the multiple target molecules are abnormal and cellular metabolic modification and/or modulation is used to treat said cellular metabolic anomalies or other pathophysiological conditions.

In yet another embodiment of the present invention, a method for the treatment of a subject who has one or more types of cancer that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif, is disclosed; where the method is comprised of the administration of the sulfur-containing, amino acid-specific small molecules of the present invention in combination with: (a) the chemotherapeutic agent cisplatin; and (b) external beam radiation in an amount sufficient to provide a therapeutic benefit to the subject suffering from one or more types of cancer that involve the abnormal biochemical activity and/or the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif.

In one embodiment of the present invention, a method for the neo-adjuvant treatment of a subject who has one or more types of cancer that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif, is disclosed; where the method is comprised of the administration of the sulfur-containing, amino acid-specific small molecules of the present invention prior to the subsequent administration of the primary chemotherapeutic regimen in an amount sufficient to provide a therapeutic benefit to the subject suffering from one or more types of cancer that involve the abnormal biochemical activity and/or the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif.

In another embodiment of the present invention, a method for the adjuvant treatment of a subject who has one or more types of cancer that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif, is disclosed; where the method is comprised of the administration of the sulfur-containing, amino acid-specific small molecules of the present invention subsequent to the administration of the initial, primary chemotherapeutic regimen in an amount sufficient to provide a therapeutic benefit to the subject suffering from one or more types of cancer that involve the abnormal biochemical activity and/or the abnormal expression of any combination of target molecules selected from the group consisting of: anaplastic lymphoma kinase (ALK), mesenchymal epithelial transition (MET) kinase, the receptor tyrosine kinase (ROS1), thioredoxin (Trx), epidermal growth factor receptor (EGFR), peroxiredoxin (Prx), excision repair cross-complementing protein 1 (ERCC1), glutaredoxin (Grx), insulin growth factor 1 receptor (IGF1R), tubulin, ribonucleotide reductase (RNR), farnesyltransferase, and other target molecules possessing a similar active site or structural motif.

In another embodiment, cancer with a T790 mutation in the epidermal growth factor receptor (EGFR) gene is disclosed.

### LISTING OF TERMS AND DEFINITIONS UTILIZED IN PRESENT PATENT APPLICATION

In addition, included is a listing of some of the terms used herein. However, it should be noted that this listing of terms and meanings set forth herein is provided solely as guidance for the reader and may be modified and/or supplemented in the subsequently-filed Utility patent application, if required.

Unless otherwise explained, all technical and scientific terms used herein have the same meaning as commonly understood by one of ordinary skill in the art to which this disclosure belongs. Although methods and materials similar or equivalent to those described herein can be used in the practice of the disclosed methods and compositions, suitable methods and materials are described below. All publications, patent applications, patents, and other references mentioned herein are incorporated by reference in their entirety. In case of conflict, the present Specification, including explanations of terms, will control. In addition, the materials, methods, and examples are for illustrative purposes only, and are not intended to be limiting.

As used herein, the term “active site” refers to a specific region of an enzyme where a substrate binds and catalysis takes place (binding site/active site). This is the region of an enzyme where the chemical reaction occurs. The active site is usually found in a 3-Dimensional groove or pocket of the enzyme, lined with amino acid residues (or nucleotides in RNA enzymes). These residues are involved in recognition of the substrate. Residues that directly participate in the catalytic reaction mechanism are called active site residues. After an active site has completed the specific chemical reaction, it can then bind another substrate molecule and catalyze another chemical reaction. Substrates bind to the active site of the enzyme through hydrogen bonds, hydrophobic interactions, temporary covalent interactions (van der Waals) or a combination of all of these to form the enzyme-substrate complex. Residues of the active site will act as donors or acceptors of protons or other groups on the substrate to facilitate the reaction. Therefore, the active site modifies the reaction mechanism in order to change the activation energy of the reaction. An enzyme binding to a substrate will lower the energy barrier that normally stops the reaction from occuring. The product of the chemical reaction is usually unstable in the active site due to steric hinderances that force it to be released, thus returning the enzyme to its initial unbound state. The induced fit theory of enzyme-substrate binding, states that the active site and the binding portion of the substrate are not exactly complementary. The induced fit model is a development of the lock-and-key model and assumes that an active site is “flexible” and it changes shape until the substrate is completely bound. The substrate is thought to induce a change in the shape of the active site. The hypothesis also predicts that the presence of certain amino acid residues in the active site will encourage the enzyme to locate the correct substrate. Conformational changes may then occur as the substrate is bound. After the products of the reaction move away from the enzyme, the active site returns to its initial conformational shape. Active sites which possess similar conformational shapes and/or amino acid sequences frequently catalyze similar substrates, e.g., kinases catalyze the phosphorylation of proteins and enzymes.

As utilized herein, the term “adenocarcinoma” refers to a cancer that originates in glandular tissue. Glandular tissue comprises organs that synthesize a substance for release such as hormones. Glands can be divided into two general groups: (i) endocrine glands—glands that secrete their product directly onto a surface rather than through a duct, often into the blood stream and (ii) exocrine glands—glands that secrete their products via a duct, often into cavities inside the body or its outer surface. However, it should be noted that to be classified as adenocarcinoma, the tissues or cells do not necessarily need to be part of a gland, as long as they have secretory properties. Adenocarcinoma may be derived from various tissues including, but not limited to, breast, colon, lung, prostate, salivary gland, stomach, liver, gall bladder, pancreas (e.g., 99% of pancreatic cancers are ductal adenocarcinomas), cervix, vagina, and uterus, as well as unknown primary adenocarcinomas. Adenocarcinoma is a neoplasm which frequently presents marked difficulty in differentiating from where and from which type of glandular tissue the tumor(s) arose. Thus, an adenocarcinoma identified in the lung may have had its origins (or may have metastasized) from an ovarian adenocarcinoma. Cancer for which a primary site cannot be found is called cancer of unknown primary.

As utilized herein, the term “adjuvant therapy” means additional treatment of a subject with cancergiven after the primary treatment or surgery to lower the risk that the cancer will come back. Adjuvant therapy may include treatment with cancer treating agents such as chemotherapeutic agents, radiation therapy, hormones, cytotoxic or cytostatic agents, antibodies and/or sulfur-containing, amino acid-specific small molecules of the present invention.

As used herein, the phrase “an amount sufficient to provide a therapeutic benefit” or “a therapeutically-effective” amount” in reference to the medicaments, compounds, or compositions of the instant invention refers to the administered dosage that is sufficient to induce a desired biological, pharmacological, or therapeutic outcome(s) in a subject suffering from one or more types of cellular metabolic anomalies or other pathophysiological conditions, including cancer. By way of non-limiting example and with regard to cancer, such outcome(s) can include: (i) cure or remission of previously observed cancer(s); (ii) shrinkage of tumor size; (iii) reduction in the number of tumors; (iv) delay or prevention in the growth or reappearance of cancer; (v) selectively sensitizing cancer cells to the activity of the anti-cancer agents; (vi) restoring or increasing apoptotic effects or sensitivity in tumor cells; and/or (vii) increasing the time of survival of the subject, alone or while concurrently experiencing reduction, prevention, mitigation, delay, shortening the time to resolution of, alleviation of the signs or symptoms of the incidence or occurrence of an expected side-effect(s), toxicity, disorder or condition, or any other untoward alteration in the subject.

As utilized herein the term “cancer” refers to all known forms of cancer including, solid forms of cancer (e.g., tumors), lymphomas, and leukemias.

As used herein, the term “cancer treating agent” or “cancer treating agents” refer to medicament(s) that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. The cancer treating agents of the present invention include, but are not limited to: (i) chemotherapeutic agents (e.g., fluropyrimidines, pyrimidine nucleosides, purine nucleosides, anti-folates, platinum agents, anthracyclines/anthracenediones, epipodophyllotoxins, camptothecins, vinca alkaloids, taxanes, epothilones, antimicrotubule agents, alkylating agents, antimetabolites, topoisomerase inhibitors, and the like); (ii) hormones, hormonal complexes, and antihormonals (e.g., interleukins, interferons, leuprolide, pegasparaginase, and the like);

(iii) enzymes, proteins, and peptides; antivirals (e.g., acyclovir, zidovudine, and the like);
(iv) cytotoxic agents, cytostatic agents; (v) polyclonal and monoclonal antibodies (e.g., crizotinib, gefitinib, erlotinib, cetuximab, afatinib, dacomitinib, ramucirumab, necitumumab, lenvatinib, palbociclib, alectinib, zybrestat, tecemotide, obinutuzumab (GA101), AZD9291, CO-1686, vintafolide, CRLX101, ipilimumab, yervoy, nivolumab, ibrutinib, selumetinib, olaparib, trastuzumab, lucitanib, rucaparib, NOV-002, MPDL3280A, pembrolizumamb, lambrolizumab (MK-3475), MEDI4736, tremelimumab, AMP-514, MEDI6469, RG7446, CRS-207, GVAX, ceritinib (LDK378), IMCgp100, vemurafenib (Zelboraf), cabozantinib, CTL019, LEE011, T-DM1, MM-121, bavituximab, MAGE-A3, axitinib, ipilimumab, rituximab, tivantinib, and the like); (vi) PD-1, PD-L1, and other checkpoint receptor inhibiting agents; (vii) immune checkpoint pathway modulatory antibodies; (viii) kinase inhibitors; (ix) ALK inhibitors; (x) cancer vaccines; (xi) Antibody Drug Conjugates; and (xii) chimeric antigen receptor T-cell (CAR-T) Therapy.

As utilized herein, the terms “cancer treating agent cycle(s)” or “cancer treating agent regimen(s)” or “chemotherapeutic regimen(s)” or “chemotherapy cycle(s)” or “treatment cycle(s)” refer to treatment using one or more of the cancer treating agents, mentioned above, with or without the use of the sulfur-containing small molecules of the present invention.

As used herein, the terms “cancer treating agent(s)” or “cancer treating drug(s)” or “cancer treating compositions” refer to a medicament or medicaments that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Cancer treating agents of the present invention include, but are not limited to: (i) chemotherapeutic agents (e.g., fluropyrimidines, pyrimidine nucleosides, purine nucleosides, anti-folates, platinum agents, anthracyclines/anthracenediones, epipodophyllotoxins, camptothecins, vinca alkaloids, taxanes, epothilones, antimicrotubule agents, alkylating agents, antimetabolites, topoisomerase inhibitors, and the like); (ii) hormones, hormonal complexes, and antihormonals (e.g., interleukins, interferons, leuprolide, pegasparaginase, and the like); (iii) enzymes, proteins, and peptides; antivirals (e.g., acyclovir, zidovudine, and the like); (iv) cytotoxic agents and cytostatic agents; (v) polyclonal and monoclonal antibodies (e.g., crizotinib, gefitinib, erlotinib, cetuximab, afatinib, dacomitinib, ramucirumab, necitumumab, lenvatinib, palbociclib, alectinib, zybrestat, tecemotide, obinutuzumab (GA101), AZD9291, CO-1686, vintafolide, CRLX101, ipilimumab, yervoy, nivolumab, ibrutinib, selumetinib, olaparib, trastuzumab, lucitanib, rucaparib, NOV-002, MPDL3280A, pembrolizumamb, lambrolizumab (MK-3475), MEDI4736, tremelimumab, AMP-514, MEDI6469, RG7446, CRS-207, GVAX, ceritinib (LDK378), IMCgp100, vemurafenib (Zelboraf), cabozantinib, CTL019, LEE011, T-DM1, MM-121, bavituximab, MAGE-A3, axitinib, ipilimumab, rituximab, tivantinib, and the like); (vi) PD-1 checkpoint receptor inhibiting agents, PD-L1 checkpoint receptor inhibiting agents, and other checkpoint receptor inhibiting agents; (vii) immune checkpoint pathway modulatory antibodies; (viii) kinase inhibitors; (ix) ALK inhibitors; (x) cancer vaccines; (xi) Antibody Drug Conjugates; and (xii) chimeric antigen receptor T-cell (CAR-T) Therapy.

As utilized herein, the terms “cancer treating agent effect” or “cancer treating agent effects” or “chemotherapeutic effect” or “cytotoxic or cytostatic activities” refer to the ability of an agent/medicament/composition to reduce, prevent, mitigate, limit, and/or delay the growth of metastases or neoplasms, or kill neoplastic cells directly by necrosis or apoptosis of neoplasms or any other mechanism, or that can be otherwise used to reduce, prevent, mitigate, limit, and/or delay the growth of metastases or neoplasms in a subject with neoplastic disease.

As utilized herein, the term “contemporaneous” refers to, e.g., an event existing, occurring, or originating during approximately the same period of time. In the instant case, and by way of non-limiting example, “contemporaneous” could refer to the sulfur-containing, amino acid-specific small molecules of the present invention interacting with and acting upon numerous target molecules in a contemporaneous manner. The term contemporaneous includes, without limitation, an event occurring, or originating simultaneously/concurrently/coincident/concomitant/or in parallel with the occurrence or origination of one or more other events.

As utilized herein, the term “cycle” refers to the administration of a complete regimen of medicaments to the patient in need thereof in a defined time period.

As used herein, the term “cytostatic agents” are mechanism-based agents that slow the progression of neoplastic disease and include drugs, biological agents, and radiation.

As used herein the term “cytotoxic agents” are any agents or processes that kill neoplastic cells and include drugs, biological agents, and radiation. In addition, the term “cytotoxic” is inclusive of the term “cytostatic”.

As used herein, the term “evidence of” as it applies to the exhibition of abnormal expression and/or abnormal biochemical activity of the target molecules of the present invention selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif, means that it is probable or likely that abnormal expression and/or abnormal biochemical activity of the target molecule(s) has occurred or will occur. It is described in that manner due to the fact that it is neither expected, nor possible to prove with 100% certainty that the cells exhibit increased expression and/or abnormal biochemical activity of the target molecules, prior to the actual treatment of the patient. By way of non-limiting example, the current use of, e.g., florescence in situ hybridization (FISH), immunohistochemistry (IHC), nucleic acid microarray analysis, radioimmunoassay (RIA), and other similar methodologies to guide treatment decisions for HER2/neu-based therapy are predicated upon the probability of the expression/increased concentrations of HER2/neu being correlated with the probability of a therapeutic response. Such expectation of a therapeutic response is not 100% certain, and is related to many factors, not the least of which is the diagnostic accuracy of the test utilized which, in turn, is also limited by the sampling of the tumor and various other factors (e.g., laboratory methodology/technique, reagent quality, and the like).

As utilized herein, the terms “Hazard Ratio”, “HR”, and “hazard ratio” refer to the chance of an event occurring with treatment “A” divided by the chance of the event occurring with treatment “B”. The hazard ratio is an expression of the hazard or chance of events occurring in one treatment arm as a ratio of the hazard of the events occurring in the other treatment arm. A hazard ratio less than 1.0 means that treatment “A” is more favorable than treatment “B” in terms of the result being measured. As described herein for purposes of data references to hazard ratios, treatment “A” refers to treatment with Tavocept (together with either paclitaxel or docetaxel and cisplatin) and treatment “B” refers to treatment with placebo (together with either paclitaxel or docetaxel and cisplatin). Accordingly, a hazard ratio less than 1.0 relating to Tavocept treatment refers to a more favorable outcome in the result being measured for Tavocept treatment in comparison to the result being measured for the treatment other than Tavocept. References to an “improvement” or “reduction” in the hazard ratio in favor of Tavocept refer to a more favorable outcome in the result being measured for Tavocept treatment in comparison to the result being measured for the treatment other than Tavocept.

As used herein, the term “heterogeneous” refers to something that is not of uniform composition, quality, or structure.

As utilized herein, the term “maintenance therapy” means the ongoing or chronic use of an agent to help lower the risk of recurrence (i.e., the return of cancer) after it has disappeared or been substantially reduced or diminished or not detectable following initial therapy or surgery. Maintenance therapy also may be used for patients with advanced cancer (cancer that cannot be cured) to help keep it from growing and spreading farther.

As used herein, the terms “modulates” or “modulation” or “metabolic modification and/or modulation” refer to any biological molecule, pharmacological medicament, or process that regulates the frequency, rate, or extent of any biological process, quality, or function. Modulation may either be “positive” (e.g., the initiation or start up of an inactive process, the maintenance of a process already occurring, the increase in rate of an existing process, and the like) or “negative” (e.g., the cessation or halting of a process with the concomitant decrease in rate, the prevention of an inactive process from becoming active, and the like).

As utilized herein, the term “neoadjuvant therapy” means treatment given as a first step to shrink a tumor before the main treatment or surgery is conducted. Neoadjuvant therapy may include treatment with cancer treating agents such as chemotherapeutic agents, radiation therapy, hormones, cytotoxic or cytostatic agents, antibodies and/or sulfur-containing, amino acid-specific small molecules of the present invention. Neoadjuvant therapy is intended to make later treatment or surgery easier and more likely to succeed, and reduce the consequences of a more extensive treatment or surgical technique that would be required if the tumor wasn't reduced in size or extent.

As used herein, the terms “pathophysiological condition” or “undesirable physiological condition” refer to abnormal anatomical or physiological conditions and their objective or subjective manifestations of disease. The term “pathophysiology” refers to the study of the biologic and physical manifestations of disease as they correlate with the underlying abnormalities and physiologic disturbances. Pathophysiology explains the processes within the body that result in the signs and symptoms of a disease.

As used herein, an “effective amount” or a “pharmaceutically-effective amount” in reference to the compounds or compositions of the instant invention refers to the amount that is sufficient to induce a desired biological, pharmacological, or therapeutic outcome in a subject with neoplastic disease. That result can be reduction, prevention, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In the present invention, the result will generally include the reduction, prevention, mitigation, delay in the onset of, attenuation of the severity of, and/or a hastening in the resolution of, or reversal of chemotherapy-associated toxicity; an increase in the frequency and/or number of treatments; an increase in duration of chemotherapeutic therapy; an increase or improvement in Progression Free Survival (PFS); and/or Complete Remission (CR).

As used herein, the term “pharmaceutically-acceptable salt” means salt derivatives of drugs which are accepted as safe for human administration. In the present invention, the sulfur-containing, amino acid-specific small molecules of the present invention include pharmaceutically-acceptable salts, which include but are not limited to: (i) a monosodium salt; (ii) a disodium salt; (iii) a sodium potassium salt; (iv) a dipotassium salt; (v) a calcium salt; (vi) a magnesium salt; (vii) a manganese salt; (viii) an ammonium salt; and (ix) a monopotassium salt.

As used herein the term “Quality of Life” or “QOL” refers, in a non-limiting manner, to a maintenance or increase in a cancer subject's overall physical and mental state (e.g., cognitive ability, ability to communicate and interact with others, decreased dependence upon analgesics for pain control, maintenance of ambulatory ability, maintenance of appetite and body weight (lack of cachexia), lack of or diminished feeling of “hopelessness”; continued interest in playing a role in their treatment, and other similar mental and physical states).

As used herein, the terms “target molecule” or “target molecules” or “molecular target” or “molecular targets” of the present invention”, refer to one or more proteins/enzymes selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target molecules possessing a similar active site or structural motif.

As used herein, the term “multiple” refers to one or more of, including by way of non-limiting example, the target molecules of the present invention which are contemporaneously modified/modulated by the sulfur-containing, amino acid-specific small molecules of the present invention.

As utilized herein, the term “non-small cell lung cancer (NSCLC)” accounts for approximately 75% of all primary lung cancers. NSCLC is pathologically characterized further into adenocarcinoma, squamous cell carcinoma, large cell carcinoma, and various other less common forms.

As used herein, the terms “abnormal expression”, “overexpression”, “overexpresses”, “abnormally elevated expression” are defined by the National Cancer Institute (NCI), as: “[i]n biology, to make either too few or too many copies of a protein or other substance. Most frequently, overexpression of certain proteins or other substances may play a role in cancer development.” See, http://www.cancer.gov/dictionary?cdrid=45812. Similarly, the Merriam-Webster Medical Dictionary definition of “overexpress” or “overespression” is given as: “[e]xcessive expression of a gene by producing too much of its effect or product. We now suspect that many, if not most, cancers arise through the overexpression of key cellular regulatory genes—J. D. Watson, et al.” See, http://www.merriam-webster.com/medical/overexpression. The term “overexpress” or “overexpression” is further defined as “[a]n abnormally high expression of a gene in comparison to the expression level of the same gene in a normal cell.” See, Cancer: Principles & Practice of Oncology, 9^{th} Edition, V. T. DeVita, T. S. Lawrence, and S. A. Rosenberg, Eds., Chapter 5—Cell Signaling, Growth Factors and Their Receptors, pg. 61. Wolters-Kluwer Medical Publishing (2010). It is extremely important to note that these aforementioned definitions make no mention of any specified, quantitative, “bright line” level of expression that is a required component to allow the practitioner to understand and/or apply the correct scientific and medical term “abnormal expression” or “overexpression” to a particular DNA, mRNA, protein, or other biological substance. Moreover, this same concept is also reflected in the peer-reviewed publications within the relevant field of art, where the terms “abnormal expression” or “overexpresses” is extensively used without any specified, quantitative, “bright line” measurement. Given these considerations, it is reasonable to believe that the meaning of the term “overexpress” or “abnormal expression” would be clear to those individuals of ordinary skill in the pertinent art, even though there was not an express reference to a specified, quantitative, “bright line” level of expression of a given DNA, mRNA, protein, or other biological substance.

In practice, a specific quantitative level of expression is not required in order for the practitioner of ordinary skill in the art to understand this term. For example, overexpression or abnormal expression is understood and identified by comparison to the expression levels found within “normal” cells. The relevant level of abnormal expression or overexpression will be impacted by the specific DNA, mRNA, protein, or other biological substance being evaluated for this abnormal expression or overexpression. Accordingly, it must be ascertained on a case-by-case basis whether a given “cell type” (i.e., obtained from a cell line, derived from a tumor biopsy, and the like) overexpresses or abnormally expresses a given DNA, mRNA, protein, or other biological substance. By way of non-limiting example, one may compare the level of expression of a specified DNA, mRNA, protein, or other biological substance of interest in a “normal” cell with the level of expression of such specified DNA, mRNA, protein, or other biological substance of interest in a cell that is deemed to be “abnormal” in order to ascertain a specific level of abnormal expression or overexpression. Some of the methodologies used to identify levels of the DNA, mRNA, and/or protein include, but are not limited to, (i) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (ii) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (iii) PCR coupled with MS approaches including, but not limited to, MALDI-TOF MS (e.g., Sequenom's MassARRAY system and related approaches); (iv) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (v) isoelectric focusing, agarose/polyacrylamide gel electrophoresis, Southern blotting, Western blotting, Northern blotting, enzyme/substrate activity assay, X-ray crystallography, and other related analytic methodologies designed to generate accurate, high-resolution data on heterogenous samples.

As used herein, the term “reducing” includes preventing, attenuating or mitigating the overall severity of, delaying the initial onset of, and/or expediting the resolution of the acute and/or chronic condition in a subject suffering from one or more types of cellular metabolic anomalies or other pathophysiological conditions.

As used herein, the phrase “seminal biological capabilities” refers to the eight (8) characteristics which are acquired by the constituent cancer during the multistep development of human tumors. These biological capabilities constitute an organizing paradigm for understanding the inherent complexities of neoplastic disease and include: (i) resisting cell death (apoptosis); (ii) enabling replicative immortality; (iii) sustaining proliferative signaling; (iv) evading growth suppressors; (v) inducing angiogenesis; (vi) activating invasion and metastasis; (vii) reprogramming of energy metabolism; and (viii) evading immune destruction. See, Hanahan, D. and Weinberg, R. A., Hallmarks of cancer: the next generation. Cell 144:646-674 (2011).

As used herein the term “structural motif” refers to a supersecondary structure found in a chain-like biological molecule, such as a protein, nucleic acid, and a variety of other molecules. Motifs do not allow the prediction of the biological functions, as they are found in proteins and enzymes with dissimilar functions. Because the relationship between the primary structure and tertiary structure is not straightforward, two biopolymers may share the same structual motif, yet lack appreciable primary structure similarity. In other words, a structural motif does not have to be associated with a sequence motif. Also, the existence of a sequence motif does not necessarily imply a distinctive structure. Structural motif elements include: (i) Beta Hairpin: two anti-parallel β-strands connected by a tight turn of a few amino acids between them; (ii) Greek Key: four β-strands folded over into a “sandwich shape”;

(iii) Omega Loop: a loop in which the residues that make up the beginning and end of the loop are very close together; (iv) Helix-Loop-Helix: consists of α-helices bound by a looping stretch of amino acids; and (v) Zinc Finger: two β-strands with an α-helix end folded over to bind a zinc ion.

As used herein, the terms “system stability” or “biological system stability” refer to the maintenance of the normative physiological state or genome level stability of the organism.

As used herein, the term “sulfur-containing, amino acid-specific small molecules of the present invention” include: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, Homocysteine-Glu, and

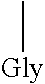

pharmaceutically-acceptable salts thereof.
The sulfur-containing small molecules of the present invention and their synthesis are described in, e.g., U.S. Pat. Nos. 5,808,160, 5,922,902, 6,160,167, and 6,504,049; and Published U.S. Patent Application No. 2005/0256055, the disclosures of which are hereby incorporated by reference in their entirety.

As used herein, the terms “Tavocept- or BNP7787-derived metabolite” or “Tavocept- or BNP7787-derived heteroconjugate” or “Tavocept- or BNP7787-derived adduct” represent the metabolite of disodium 2,2′-dithio-bis-ethane sulfonate, 2-mercapto ethane sulfonate sodium as a disulfide form which is conjugated with a substituent group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu; and

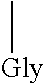

pharmaceutically-acceptable salts thereof.
The aforementioned “Tavocept- or BNP7787-derived metabolite” or “Tavocept- or BNP7787-derived heteroconjugate” compounds are included in the sulfur-containing, amino acid-specific small molecules of the present invention and may be synthesized as described in U.S. Pat. Nos. 7,829,117; 7,829,538; 7,829,539; 7,829,540; and 7,829,541, the disclosures of which are incorporated herein, by reference, in their entirety.

As used herein, the term “Tavocept” refers to disodium 2,2′-dithio-bis-ethane sulfonate, and is also referred to in the literature as dimesna and BNP7787.

As used herein, the term “treat” or “treated”, with respect to a subject without cancer, refers to a subject, who is in need thereof, and who has received, is currently receiving, or will receive the sulfur-containing, amino acid-specific small molecules of the present invention.

As used herein, the term “treat” or “treated”, with respect to a subject with cancer, refers to a subject, who is in need thereof, and who has received, is currently receiving, or will receive one or more cancer treating agents and/or the sulfur-containing, amino acid-specific small molecules of the present invention and/or other treatment agents.

As used herein, the term “xenobiotic” refers to or denotes a substance, typically a synthetic chemical, which is found in an organism but which is not normally produced or expected to be present in said organism. The term can also refer to substances which are present in much higher concentrations than are usual.

As used herein, the terms “xenobiotic modification”, “Tavocept-mediated xenobiotic modification”, or a “Tavocept mediated, non-enzymatic xenobiotic modification” refer to the covalent binding of a “Tavocept- or BNP7787-derived metabolite” or “Tavocept- or BNP7787-derived heteroconjugate” or “Tavocept- or BNP7787-derived adduct” to one or more sulfur-containing amino acids of a protein or enzyme.

### DESCRIPTION OF THE FIGURES

FIG. 1: illustrates the ability of Tavocept to undergo thiol disulfide exchange reactions intracellularly and/or within the interstitial space.

FIG. 2: illustrates the chemical structures of Tavocept, 2-mercaptoethene sulfonate, glutathione, and selected Tavocept-derived heteroconjugates. It should be noted that in the structures of the Tavocept-derived heteroconjugates, the portion of the heteroconjugates comprising the Tavocept metabolite, 2-mercaptoethene sulfonate, is shaded.

FIG. 3: Panel A: Glutaredoxin (Grx) catalyzes the reduction of disulfide bonds in proteins converting glutathione (GSH) to glutathione disulfide (GSSG). GSSG is, in turn, recycled to GSH by the enzyme glutathione reductase at the expense of NADPH. During the reaction cycle it is thought that a cysteine pair in the active site of glutaredoxin is converted to a disulfide. Panel A: illustrates the conversion of glutaredoxin from the disulfide form (oxidized) to the dithiol (reduced) form, as catalyzed non-enzymatically by glutathione. Panel B: glutaredoxin is also thought to be important for deglutathionylation of protein thiols. In this reaction only a single cysteine is required. Indeed, many naturally occurring glutaredoxins contain only one cysteine in the active site. It should be noted that the direction of the glutaredoxin-catalyzed cycle depends on the relative concentrations of GSH and GSSG. High concentrations in the cell of GSSG relative to GSH will drive glutathionylation or the oxidation of protein thiols to disulfides.

FIG. 4: illustrates an example of the Xenobiotic Metabolism Pathway. Tavocept is thought to react with cisplatin resulting in the formation of a mesna-cisplatin adduct that is not a substrate for the xenobiotic metabolism pathway.

FIG. 5: illustrates the domains of cMet; the kinase domain of MET (residues 956-end) was used in these experiments.

FIG. 6: illustrates the domain organization of MET. Panel A: illustrates the domain organization and structure. Panel B: illustrates a slightly modified MET showing sites for tyrosine phosphorylation in the intracellular kinase portion of MET.

FIG. 7: illustrates increasing concentrations of MET result in increasing ADP production (reflected in increasing RLU). Assay volume was 10 μL; therefore, for the assay represented by the 0.78 ng bar above, MET Kinase was 0.078 ng/μL and this corresponded to a molar concentration of 0.96 nM.

FIG. 8: illustrates the effect of Tavocept (BNP7787) on MET (0.1 ng/μL) activity in assays with 10 μM ATP; Determination of IC₅₀ value.

FIG. 9: illustrates the effect of Tavocept (BNP7787) on MET (0.1 ng/μL) activity in assays with 100 μM ATP; Determination of IC₅₀ value.

FIG. 10: illustrates the effect of Tavocept (BNP7787) on MET (2.5 ng/μL) activity in assays with 100 μM ATP; Determination of IC₅₀ value.

FIG. 11: illustrates the effect of Tavocept (BNP7787) on MET (2.5 ng/μL) activity in assays with 10 μM ATP; Determination of IC₅₀ value.

FIG. 12: illustrates the effect of Crizotinib on MET (0.1 ng/μL) activity in assays with 10 μM ATP; Determination of IC₅₀ value.

FIG. 13: illustrates the effect of Crizotinib effect on MET (2.5 ng/μL) activity in assays with 100 μM ATP; Determination of IC₅₀ value.

FIG. 14: illustrates the effect of Tavocept (BNP7787) on Crizotinib-mediated inhibition of MET (0.1 ng/μL) activity under 10 μM ATP conditions at 20 nM and 40 nM Crizotinib.

FIG. 15: illustrates the effect of Tavocept (BNP7787) on Crizotinib-mediated inhibition of MET (2.5 ng/μL) activity under 100 μM ATP conditions at 45 nM and 90 nM Crizotinib.

FIG. 16: illustrates the effect of Staurosporine on MET (0.1 ng/μL) activity in assays with 10 μM ATP; Determination of IC₅₀ value.

FIG. 17: Illustrates the effect of Tavocept (BNP7787) on Staurosporine-mediated inhibition of MET (0.1 ng/μL) activity under 10 μM ATP conditions at a 100 nM and 300 nM concentration of Staurosporine.

FIG. 18: Panel A: illustrates a ribbon diagram of ALK with covalently bound Tavocept (BNP7787)-derived mesna adducts. Tavocept (BNP7787)-derived mesna adducts were observed at Cys 1235 and Cys1156. Panel B: illustrates an overlay of region of apo-ALK with Tavocept (BNP7787) xenobiotically-modified ALK that has a Cys-1156-mesna adduct. The Tavocept (BNP7787)-derived mesna adduct occupies the same pocket at Phe1127 of the P-loop.

FIG. 19: illustrates a Fo-Fc electron density map contoured at 1 sigma showing Tavocept (BNP7787)-derived mesna adducts on ALK. Panel A: at Cys 1235. Panel B: at Cys 1156. Panel C: Binding site of the Tavocept (BNP7787)-derived mesna adduct at Cys 1235. There are no obvious interactions with the protein other than the covalent bond with Cys 1235. Panel D: Molecular surface of ALK with the Tavocept (BNP7787)-derived mesna at Cys 1156 removed to show the interaction of the adduct with the protein. A water mediated hydrogen bond is present between the mesna sulfonate and Asp 1160 carbonyl.

FIG. 20: illustrates the X-ray crystallographic structure of ALK with Tavocept (BNP7787)-derived mesna adducts on cys1156 and cys1235.

FIG. 21: illustrates the kinase domain of ALK (residues 1058-1623) which was used in these experiments.

FIG. 22: illustrates increasing concentrations of ALK result in increasing ADP production (reflected in increasing RLU).

FIG. 23: Panel A: illustrates Crizotinib's effect on ALK activity in assays with 100 μM ATP; Panel B: illustrates a summary of IC₅₀ value determination.

FIG. 24: Panel A: illustrates Crizotinib's effect on ALK activity in assays with 500 μM ATP; Panel B: illustrates a summary of IC₅₀ value determination.

FIG. 25: illustrates the effect of Tavocept (BNP7787) on Crizotinib-mediated inhibition of ALK activity under 100 μM ATP conditions and 15 nM Crizotinib (Panel A) or 30 nM Crizotinib (Panel B).

FIG. 26: illustrates the effect of Tavocept (BNP7787) on Crizotinib-mediated inhibition of ALK activity under 500 μM ATP conditions at 30 nM Crizotinib (Panel A) or 65 nM Crizotinib (Panel B).

FIG. 27: illustrates a homology model of human ROS1 overlaid with the X-ray crystallographic structure of human ALK (Protein Data Bank (PDB) entry for ALK was 3 L9P).

FIG. 28: illustrates the domain organization of ROS1. Panel A: Domain organization of ROS1 compared to other kinase receptors. Below each kinase, genes are listed that can fuse with the kinase (fused products may be involved in cancer or disease). Panel B: Intracellular kinase region of ROS1 including residues 1883-2347 with tyrosine (Y) and serine (S) phosphorylation sites identified.

FIG. 29: illustrates that increasing concentrations of ROS1 results in increasing ADP production (reflected in increasing RLU). It should be noted that above a concentration of 1 ng/μL assay, ATP becomes rate limiting; therefore a lower ROS1 concentration of 0.5 ng/μL assay was utilized with an ATP concentration of 100 μM.

FIG. 30: illustrates the effects of crizotinib on ROS1 activity. Assays with 100 μM ATP: Determination of IC₅₀ value assays contained 100 μM ATP and 0.5 ng ROS1/μL assay volume, the data is shown to two decimal places. Panel A: Experiment No. 1. Panel B: Experiment No. 2. Panel C: Summary of IC₅₀ values determined using Origin Lab Software.

FIG. 31: illustrates the time-dependent decrease in ROS1 activity when Tavocept (BNP7787) and ROS1 are incubated together prior to initiating the kinase assay. Panel A: ROS1 and Tavocept (BNP7787) assayed immediately in the presence of assay mixture containing ATP and polyGT. Panel B: ROS1 and Tavocept (BNP7787) incubated together for 3 hours, then added to assay mixture containing ATP and polyGT and assayed. Panel C: ROS1 and Tavocept (BNP7787) preincubated together for 24 hours, then added to assay mixture containing ATP and polyGT and assayed.

FIG. 32: illustrates the time-dependent decrease in ROS1 activity when Tavocept (BNP7787) and ROS1 are incubated together prior to initiating the kinase assay. Panel A: ROS1 and Tavocept (BNP7787) assayed immediately in the presence of assay mixture containing ATP and polyGT. Panel B: ROS1 and Tavocept (BNP7787) incubated together for 3 hours, then added to assay mixture containing ATP and polyGT and assayed. Panel C: ROS1 and Tavocept (BNP7787) preincubated together for 24 hours, then added to assay mixture containing ATP and polyGT and assayed.

FIG. 33: illustrates the effect of Tavocept (BNP7787) on Crizotinib-mediated inhibition of ROS1 kinase activity in assays with simultaneous addition of all assay components Tavocept (BNP7787), ATP, and polyGT were added to ROS1 kinase simultaneously. In this experiment ROS1 kinase was not preincubated with Tavocept (BNP7787) prior to initiation of the kinase assay.

FIG. 34: illustrates the effect of BNP7787 on Crizotinib-mediated inhibition of ROS1 kinase activity in assays where BNP7787 is incubated with ROS1 kinase prior to addition of ATP and polyGT. BNP7787 was found to have an additive effect on crizotinib-induced inhibition of ROS1 kinase activity. BNP7787 was preincubated with ROS1 kinase for: Panel A: 0 hours; Panel B: 3 hours; or Panel C: 24 hours prior to addition of crizotinib, ATP, and polyGT.

FIG. 35: Panel A: Tavocept effect on Wild Type EGFR activity in assays with 10 μM ATP concentration; Panel B: Summary of IC50 Value Determination.

FIG. 36: illustrates Tavocept effect on T790M EGFR activity in assays with 10 μM ATP concentrations.

FIG. 37: illustrates the structures of the Tavocept-derived heteroconjugates evaluated in EGFR Kinase assays.

FIG. 38: illustrates Tavocept potentiation of Erlotinib-mediated inhibition of WT EGFR Kinase activity (10 μM ATP).

FIG. 39: illustrates Tavocept potentiation of Erlotinib-mediated inhibition of T790M EGFR Kinase activity (10 μM ATP).

FIG. 40: illustrates the effect of Tavocept-derived heteroconjugates on Erlotinib-mediated inhibition of WT EGFR activity under 10 μM ATP conditions. Panel A: Effect of mesna-cysteine; Panel B: Effect of mesna-glutathione; Panel C: Effect of mesna-cysteinylglutamate; and Panel D: Effect of mesna-cysteinylglycine.

FIG. 41: illustrates the effect of Tavocept-derived heteroconjugates on Erlotinib-mediated inhibition of WT EGFR activity under 100 μM ATP conditions. Panel A: Effect of mesna-cysteine; Panel B: Effect of mesna-glutathione; Panel C: Effect of mesna-cysteinyl glutamate; Panel D Effect of mesna-cysteinyl glycine.

FIG. 42: illustrates the effect of Tavocept-derived heteroconjugates to potentiate the inhibitory effect of Erlotinib on T790M EGFR activity (10 μM ATP). Panel A; Mesna-cysteine and Mesna-glutathione inhibition of T790M EGFR and potentiation of Erlotinib inhibition (10 μM ATP); Panel B: Mesna-cysteinylglycine and mesna-cysteinylglutamate inhibition of T790M EGFR and potentiation of Erlotinib inhibition (10 μM ATP).

FIG. 43: illustrates the effect of Tavocept-derived heteroconjugates potentiating the inhibitory effect of Erlotinib on T790M EGFR activity (100 μM ATP). Panel A: MSSC potentiates Erlotinib-mediated inhibition of T790M EGFR kinase activity (100 μM ATP); Panel B: MSSGlutathione potentiates Erlotinib-mediated inhibition of T790M EGFR kinase activity (100 μM ATP); Panel C: MSSCysteinylglycine potentiates Erlotinib-mediated inhibition of T790M EGFR kinase activity (100 μM ATP); Panel D: MSSCysteinylglutamate potentiates Erlotinib-mediated inhibition of T790M EGFR kinase activity (100 μM ATP).

FIG. 44: A graphic illustration of the intracellular pathways related to IGF (as adopted from Fidler, et al. Targeting the insulin-like growth factor receptor pathway in lung cancer: Problems and pitfalls. Ther. Adv. Med. Oncol. 4(2):51-60 (2012)).

FIG. 45: illustrates the effect of Tavocept on IGF1R Kinase activity.

FIG. 46: illustrates the amino acid sequence of human ERCC1 (cysteines are underlined). The N-terminal 6-histidine tag (HHHHHH) used to express human ERCC1 in E. coli is not shown. The ERCC1 sequence was obtained from http://www.uniprot.org/uniprot/P07992.

FIG. 47: Panel A: Positive ion ESI mass spectrum of ERCC1 control sample corresponding to tryptic ERCC1 fragment VTECLTTVK containing Cys238. Panel B: Positive ion ESI mass spectrum of ERCC1 Tavocept-treated sample corresponding to tryptic ERCC1 fragment VTECLTTVK containing a Tavocept-derived mesna adduct on Cys238.

FIG. 48: Panel A: Positive ion ESI mass spectrum of ERCC1 control unmodified sample corresponding to tryptic ERCC1 fragment EDLALCPGLGPQK containing Cys274. Panel B: Fragment from ERCC1 control contains EDLALCPGLGPQK fragment that contains Tavocept modification on Cys274 (predicted 1478.6; observed 1480.8).

FIG. 49: Whole Protein MS data showing—Panel A: Apo-RNR1. Panel B: RNR1 with Tavocept-derived mesna adducts.

FIG. 50: (A) Tavocept structure; (B) Paclitaxel structure; (C) Cisplatin structure and subsequent aquation leading to formation of cisplatin-DNA adducts that interfere with DNA replication and damage DNA.

FIG. 51: Panel A: Example of time dependent decay of microtubule protein's ability to polymerize into microtubules (control sample with no drug treatment). Percent polymerization values are OD₃₅₀ readings 30 minutes after polymerization was initiated and are normalized relative to the sample that was not preincubated prior to initiation of the MTP polymerization assay (the 0 hour sample). Note: individual microtubule protein preparations vary slightly in terms of decay profiles). Panel B: Bar graph comparison of percent polymerization of microtubule protein after preincubation with mesna only, monoaquocisplatin only, and mesna with monoaquocisplatin. Percent polymerization values are OD₃₅₀ readings from 30 minutes after polymerization was initiated. At each time point the regular pH control is assigned a 100% polymerization value and the remaining samples are normalized relative to that sample. Final assay concentrations were mesna (200 μM) and monoaquocisplatin (36 μM).

FIG. 52: Panel A: Postulated S_{N}2 route of non-enzymatic reduction of Tavocept to mesna in the kidney (see, e.g., Verschraagen M, Boven E, Torun E, Hausheer F H, Bast A, van dV. Possible enzymatic routes and biological sites for metabolic reduction of BNP7787, a new protector against cisplatin-induced side-effects. Biochem. Pharmacol. 68:493-502 (2004)). Panel B: Mesna may displace the aquo group of monoaquocisplatin and the formation of a possible sulfur-platinum adduct could prevent monoaquoplatin from forming an adduct with surface cysteine residues on tubulin.

FIG. 53: Effects of Tavocept and mesna on microtubule protein polymerization under various assay conditions. Panel A: Tavocept has a dose-dependent inhibitory effect on GTP driven microtubule protein polymerization (the data in Panel A was obtained using a Cary100 UV-vis cuvette based spectrometer). Panel B: Mesna does not affect GTP Promoted Microtubule Protein Polymerization (the data in Panel B was obtained using a SpectraMax Plus microtiter plate UV-Vis spectrophotometer). Panel C: Tavocept modulates GTP/paclitaxel-promoted microtubule protein polymerization (note—the line with open squares is a microtubule protein polymerization assay promoted only by GTP, a GTP-only control). Panel D: Tavocept modulates paclitaxel-promoted microtubule protein polymerization (no GTP present) and this effect is not due to the two sodium counterions of Tavocept, since 32 mM NaCl alone was shown to have no effect.

FIG. 54: Electron micrographs of microtubule polymerization—Panel A: initiated with GTP; Panel B: initiated with GTP with Tavocept (10 mM) present; Panel C: initiated with GTP with paclitaxel (6 μM); and Panel D: initiated with GTP with paclitaxel (6 μM) and with Tavocept (10 mM) present.

FIG. 55: illustrates Liquid Chromatography data of peroxiredoxin fragment HGEVCPAGWK containing Cys173. Panel A: peroxiredoxin incubated with Tavocept ion at m/z 1245.5 corresponding to fragment [HGEVCPAGWK+H]+; Panel B: peroxiredoxin incubated without Tavocept does not have ion at m/z 1245.5 and exhibits no peaks corresponding to this mass.

FIG. 56: positive-ion mass spectra for peroxiredoxin (Prx) fragments containing Tavocept-derived mesna adduct at Cys173 in fragment TDKHGEVCPAGW. Panel A: peroxiredoxin incubated with Tavocept; ion at m/z 1439.8 corresponding to fragment [TDKHGEVCPAGW+H]+ containing a mesna moiety; Panel B: peroxiredoxin incubated without Tavocept does not have ion at m/z 1439.8 but contains “parent” unmodified [TDKHGEVCPAGW+H]+ ion at m/z 1300.9.

FIG. 57: positive-ion mass spectra for peroxiredoxin (Prx) fragment VCPTEIIAF containing Cys52. Panel A: peroxiredoxin incubated with Tavocept; ion at m/z 1132.8 corresponding to fragment [VCPTEIIAF+H]+; Panel B: peroxiredoxin incubated without Tavocept does not have ion at m/z 1132.8 but contains “parent” unmodified peak at 992.0.

FIG. 58: illustrates Prx assay that was coupled to thioredoxin (Trx), thioredoxin reductase (TrxR), and NADPH.

FIG. 59: illustrates that Tavocept-modified Prx (Prx-mesna) is less active than Apo-Prx in assays monitoring initial velocity.

FIG. 60: crystal structure of Prx4 complexed with Tavocept-derived mensa moiety showing analogue formation at Cys124 but not Cys148. Cys245 was not visible in the electron density map.

FIG. 61: illustrates Prx apo structure showing multimer interface. The C-terminal “tail” of molecule A wraps around molecule B such that Cys 124 of Molecule B is in close proximity to Cys245 of Molecule A.

FIG. 62: close-up of Tavocept-derived mesna binding site showing partial unwinding of helix 124 and unwinding of helix 165 to accommodate the Tavocept-derived mesna moiety at C124.

FIG. 63: illustrates the Mass Spectroscopy analysis of Prx Protein after reaction with Tavocept. Peak at 25572 corresponds to Prx monomer containing two Tavocept-derived mesna adducts (apo-protein is approximately 25292; the peak at 25751 corresponds to Prx with three Tavocept-derived mesna adducts); the peak at 51041 corresponds to dimer containing up to three Tavocept-derived mesna adducts.

FIG. 64: illustrates the Prx protein sample after crystallization; crystals were dissolved for Mass Spectroscopy analysis to confirm the C-terminal tail was intact. Observed peak at 25574 corresponds extremely well to the initially observed peak at 25572, suggesting no C-terminal proteolysis has occurred.

FIG. 65: illustrates the sequence alignment of Prx 1 and Prx 4 (Prx IV) showing position of the conserved, catalytic cysteine residues in boxes.

FIG. 66: Electrophoretic profile of Trx incubated with and without Tavocept. Panel A: TrisGlycine SDS PAGE, reducing (DTT) conditions. Panel B: TrisGlycine Native PAGE, non-reducing conditions. Panel C: TrisGlycine Native PAGE, reducing (DTT) conditions. Lane 1 contains See Blue Plus Two Standards (Panel A) NativeMarks (Panels B and C). Due to steps required in the technical manipulation of samples as they are prepared for PAGE, 0 hrs samples are actually closer to a 15 to 30 minute time-point. Freezing samples for up to 3 weeks gave identical PAGE profiles as when samples were analyzed immediately; therefore, summary gels showing the various time points are presented here.

FIG. 67: IEF PAGE Electrophoretic profile of Trx incubated with and without Tavocept. Panel A: IEF gel. Panel B: IEF gel where all samples were incubated with Trx reductase and NADPH prior to loading. Lane 1 contains IEF protein standards (Panels A and B). Due to steps required in the technical manipulation of samples as they are prepared for PAGE, 0 hour samples are actually closer to a 15 to 30 minute time-point. Freezing samples for up to 3 weeks gave identical PAGE profiles as when samples were analyzed immediately as they were generated; therefore, summary gels showing the various time-points are presented here.

FIG. 68: Liquid Chromatography data of thioredoxin incubations. Panel A: Thioredoxin incubated without Tavocept; ion at m/z 1148.3 with retention time of 12.76 minutes corresponding to fragment [CMPTFQFFK+H]⁺. Panel B: Thioredoxin incubated without Tavocept does not have ion at m/z 1288.3 and exhibits no peaks corresponding to this mass (note: 1288.3 corresponds to [CMPTFQFFK+Mesna+H]⁺). Panel C: Thioredoxin incubated with Tavocept; ion at m/z 1288.3 with retention time of 13.26 minutes corresponding to fragment [CMPTFQFFK+Mesna+H]⁺ with Cys73-mesna adduct. Panel D: Thioredoxin incubated with mesna; ion at m/z 1288.3 with retention time of 13.26 minutes corresponding to fragment [CMPTFQFFK+Mesna+H]⁺ with Cys73-mesna adduct.

FIG. 69: Mass Chromatography data of thioredoxin incubations. Panel A: The positive-ion mass spectrum for thioredoxin fragments containing covalent mesna adducts from reactions where thioredoxin was incubated with Tavocept for Cys73-mesna adduct at ions of m/z 1288.3 [CMPTFQFFK+Mesna+H]⁺ and m/z 1310.2 [CMPTFQFFK+Mesna+Na+H]⁺, respectively. Panel B: The negative-ion mass spectrum for thioredoxin fragments containing covalent mesna adducts from reactions where thioredoxin was incubated with Tavocept for identification of Cys73-mesna adducts at the ion of m/z 1286.3 [CMPTFQFFK+Mesna−H]. Panel C: The positive-ion mass spectrum for thioredoxin fragments containing covalent mesna adducts from reactions where thioredoxin was incubated with Tavocept for identification of a mesna adduct in Trypsin digested thioredoxin fragment containing Cys62 and Cys69 at ions of m/z 2718.6 [YSNVIFLEVDVDDCQDVASECEVK+H]⁺ and m/z 2860.4 [YSNVIFLEVDVDDCQDVASECEVK+Mesna+H]⁺, respectively.

FIG. 70 Summary of effect of Tavocept and Tavocept-derived mesna-disulfide heteroconjugates on Trx activity. Panel A: Tavocept and Tavocept-derived mesna-disulfide heteroconjugates are alternative substrates for the TrxR/Trx system, and as such can act as competitive inhibitors of the Trx. NADPH oxidation by TrxR and Trx (see, Table 6, Reaction B conditions) increases in the presence of increasing concentrations of Tavocept, MSSG, MSSC, and MSSH. (Note: error bars are small and may be obscured by the symbols). Panel B: Tavocept has a discernable effect on the rate of NADPH oxidation in the TrxR/Trx catalyzed reduction of the insulin AB chain disulfide and more notably increasing Tavocept concentrations prevent most of the precipitation of the insulin B chain when the AB chain disulfide is cleaved. (Note: error bars were omitted but assays were run in quadruplicate with typical errors of 5-8% between individual replicates). Panel C: Progress curves of assays where Trx-mesna and Trx-GSH were purified away from unreacted Tavocept and glutathione disulfide, respectively, and then compared to apo-Trx in the Trx reductase insulin disulfide reduction assay. Panel D: Initial velocity reaction rates corresponding to progress curves shown in Panel C. With time, Trx-mesna and Trx-GSH are converted back to apo-Trx by Trx reductase (see also, FIG. 69, Panel E).

FIG. 71: Molecular assembly of the Trx tetramer. All three Trx crystals were highly similar in overall fold conformation, the Trx pH 9.0/8.5 tetramer is shown in this figure (i.e., adduct formation at pH 9.0; crystal grown at pH 8.5). Panel A: Ribbon diagram of tetramer with the site of conformational change (light color) for molecules A and B and (darker color) for C and D, respectively. Panel B: The intra- and intermolecular disulfide pattern is illustrated.

FIG. 72: Molecular interface of the Trx structure containing Tavocept-derived mesna adduct. Panel A: Close-up of the tetramer interface showing the formation of a six stranded β-barrel at the dimer interface of molecules C and D. Panel B: View down the “barrel” of the dimer interface showing the newly formed disulfides. Panel C: Representative 2Fo-Fc electron density map contoured at 1 sigma at the β-barrel motif (Nomenclature note: Cys69-D indicates Cys69 of molecule D, etc.). Geometry of tetramer interface was similar in all three crystal structures; here crystals obtained at pH 8.5 with adduct formed at pH 9.0 are shown.

FIG. 73: Scheme showing Tavocept-derived modification of proteinaceous cysteine residues resulting in formation of mixed mesna-cysteine disulfides on protein targets. This process is called Tavocept-mediated xenobiotic modification or modulation.

FIG. 74: Mass Spectrum showing that Human Grx contains two Tavocept-derived mesna adducts (Human Grx mol weight=11,930 g/mol; add 2 mesna groups at 141 g/mol to obtain approximately 12, 212 molecular weight peak—see peak at 12,210).

FIG. 75: Atomic resolution map of Tavocept-derived mesna adduct formation on Cys7 (Panel A) and Cys82 (Panel B). The adduct on Cys7 is in one conformation. The adduct on Cys82 is shown in two conformations for S—Cβ bond of the adduct.

FIG. 76: Ribbon diagram showing the binding site of the two Tavocept-derived adducts to Grx1. Panel A: Tavocept-derived mesna adduct at Cys82. The sulfur and carbon atoms of the mesna are in two orientations. The sulfate is in a single orientation and making a hydrogen bond with Ser83 (also in two orientations). Panel B: Tavocept-derived mesna adduct at Cys7. This adduct is solvent exposed and not making any interactions with the protein. Both adducts are located at a crystal contact.

FIG. 77: Overlay of BNPI proprietary Grx structure with PDB entry 1KTE (rms 0.562). Tavocept adducts are depicted as sticks on the Grx structure.

FIG. 78: Overview of some intracellular pathways regulated or modulated by RAS. Adapted from Appels, et al., Development of Farnesyl Transferase Inhibitors: A Review. 10:565-578 (2005).

FIG. 79: Inhibition of FTase activity by Tavocept. Panel A: Progress curve showing FTase-mediated farnesylation of Dansyl-GCVLS peptide. Panel B: Relative rates from reactions shown in Panel A.

FIG. 80: FTase assay summary.

FIG. 81: Scheme showing possible interactions between Tavocept and Dansyl-GCVLS peptide.

FIG. 82: Summary of Mass Spectroscopy data showing that Tavocept reacts with the Dansyl-GCVLS peptide resulting in the formation of a xenobiotically modified Dansyl-GCVLS substrate.

### DETAILED DESCRIPTION OF THE INVENTION

The descriptions and embodiments set forth herein are not intended to be exhaustive, nor do they limit the present invention to the precise forms disclosed. They are included to illustrate the principles of the invention, and its application and practical use by those skilled in the art.

### Multiple Protein Targets and Experimental Results

A. Tyrosine Kinases

The term kinase describes a large family of enzymes that are responsible for catalyzing the transfer of a phosphoryl group from a nucleoside triphosphate donor, such as ATP, to an acceptor molecule. Tyrosine kinases catalyze the phosphorylation of tyrosine residues in proteins. The phosphorylation of tyrosine residues, in turn, cause a change in the function of the protein that they are contained in. Phosphorylation at tyrosine residues controls a wide range of properties in proteins such as enzyme activity, subcellular localization, and interaction between molecules.

Tyrosine kinases function in a variety of processes, pathways, and actions, and is responsible for key events in the body. The receptor tyrosine kinases function in transmembrane signaling; whereas tyrosine kinases within the cell function in signal transduction to the nucleus. Tyrosine kinase activity in the nucleus involves cell-cycle control and properties of transcription factors. In this way, in fact, tyrosine kinase activity is involved in mitogenesis, or the induction of mitosis in a cell; proteins in the cytosol and proteins in the nucleus are phosphorylated at tyrosine residues during this process. Cellular growth and reproduction may rely in some part on tyrosine kinase. Tyrosine kinase function has been observed in the nuclear matrix, which is comprised not of chromatin, but of the nuclear envelope and a “fibrous web” that serves to physically stabilize DNA. The transmission of mechanical force, regulatory signaling, and cellular proliferation are fundamental in the normal survival of a living organism and protein tyrosine kinases also play a role in these functions.

Tyrosine kinases also function in many signal transduction cascades wherein extracellular signals are transmitted through the cell membrane to the cytoplasm and often to the nucleus where gene expression may be modified. See, e.g., Cox, Michael; Nelson, David R. (2008). Lehninger: Principles of Biochemistry (5^{th} edition). W. H. Freeman & Co. Signals in the surroundings received by receptors in the membranes of cells are transmitted into the cell cytoplasm. Transmembrane signaling due to receptor tyrosine kinases relies heavily upon interactions, for example, mediated by the SH2 protein domain; it has been determined via experimentation that the SH2 protein domain selectivity is functional in mediating cellular processes involving tyrosine kinase. Receptor tyrosine kinases may, by this method, influence growth factor receptor signaling. Finally mutations can cause some tyrosine kinases to become constitutively active, a nonstop functional state that may contribute to initiation or progression of cancer.

### Tyrosine Kinase Families

Tyrosine kinases are divided into two main families: (i) transmembrane receptor-linked kinases and (ii) cytplasmic proteins. Approximately 2000 kinases are known, and more than 90 protein tyrosine kinases (PTKs) have been identified in the human genome. They are divided into two classes, receptor and non-receptor PTKs. At present, 58 receptor tyrosine kinases (RTKs) are known, and grouped into 20 subfamilies. RTKs play pivotal roles in diverse cellular activities including growth, differentiation, metabolism, adhesion, motility, and cellular death. RTKs are composed of an extracellular domain, which is able to bind a specific ligand, a transmembrane domain, and an intracellular catalytic domain, which is able to bind and phosphorylate selected substrates. Binding of a ligand to the extracellular region causes a series of structural rearrangements in the RTK that lead to its enzymatic activation. In particular, movement of some parts of the kinase domain gives free access to adenosine triphosphate (ATP) and the substrate to the active site. This triggers a cascade of events through phosphorylation of intracellular proteins that ultimately transmit (i.e., “transduce”) the extracellular signal to the nucleus, causing changes in gene expression. Many RTKs are involved in oncogenesis, either by gene mutation, or chromosome translocation, or simply by over-expression. In every case, the result is a hyper-active kinase, that confers an aberrant, ligand-independent, non-regulated growth stimulus to the cancer cells.

In humans, a total of 32 cytoplasmic/non-receptor protein tyrosine kinases have been identified. The first non-receptor tyrosine kinase identified was the v-src oncogenci protein. Most animal cells contain one or more members of the Src family of tyrosine kinases. A chicken sarcoma virus was found to carry mutated versions of the normal cellular Src gene. The mutated v-src gene has lost the normal built-in inhibition of enzyme activity that is characteristic of cellular Src (c-src) genes. Src family members have been found to regulate many cellular processes. For example, the T-cell antigen receptor leads to intracellular signalling by activation of Lck and Fyn, two proteins that are structurally similar to Src.

### Regulation of Tyrosine Kinases

Major changes are sometimes induced when the tyrosine kinase enzyme is affected by other factors. One of the factors is a molecule that is bound reversibly by a protein, called a ligand. A number of receptor tyrosine kinases, though certainly not all, do not perform protein-kinase activity until they are occupied, or activated, by one of these ligands. It is interesting to note that, although many more recent cases of research indicate that receptors remain active within endosomes, it was once thought that endocytosis caused by ligands was the event responsible for the process in which receptors are inactivated. Activated receptor tyrosine kinase receptors are internalized in short time and are ultimately delivered to lysosomes, where they become work adjacent to the catabolic acid hydrolases that partake in digestion. Internalized signaling complexes are involved in different roles in different receptor tyrosine kinase systems, the specifics of which have been examined. See, e.g., Wiley H. S., Burke, P. M. Regulation of receptor tyrosine kinase signaling by endocytic trafficking Traffic 2(1):12-18 (2001). Additionally, ligands participate in reversible binding, a term that describes those inhibitors that bind non-covalently (inhibition of different types are effected depending on whether these inhibitors bind the enzyme, the enzyme-substrate complex, or both). Multivalency, which is an attribute that bears particular interest to some people involved in related scientific research, is a phenomenon characterized by the concurrent binding of several ligands positioned on one unit to several coinciding receptors on another. In any case, the binding of the ligand to its partner is apparent owing to the effects that it can have on the functionality of many proteins. Ligand-activated receptor tyrosine kinases, as they are sometimes referred to, demonstrate a unique attribute. Once a receptor tyrosine kinase is bonded to its ligand, it is able to bind to tyrosine kinase residing in the cytosol of the cell.

(i) Mesenchymal Epithelial Transition (MET) Kinase

The MET proto-oncogene encodes for the receptor tyrosine kinase (RTK), c-MET. MET encodes a protein known as hepatocyte growth factor receptor (HGFR). The hepatocyte growth factor receptor protein possesses tryrosine kinase activity. See, e.g., Cooper, C. S., The MET oncogene: from detection by transfection to transmembrane receptor for hepatocyte growth factor. Oncogene 7(1):3-7 (1992). c-MET is a membrane receptor that is essential for embryonic development and tissue repair (e.g., wound healing). Hepatocyte growth factor (HGF) is the only known ligand of the c-MET receptor. MET is normally expressed in cells of epithelial origin, although it has also been identified in endothelial cells, neurons, hepatocytes, hematopoietic cells, and melanocytes. Expression of HGF is generally restricted to cells of mesenchymal origin, although some epithelial cancer cells appear to express both HGF and MET.

The MET proto-oncogene has a total length of 125,982 bp and is located in the 7q31 locus of chromosome 7. MET is transcribed into a 6,641 bp mature mRNA which is then translated into a 1,390 amino acid residue c-MET protein. c-MET is a receptor tyrosine kinase that is produced as a primary single-chain precursor protein that is post-translationally proteolytically cleaved at a furin site to yield a highly glycosylated extracellular α-subunit and a transmembrane β-subunit, which are then covalently linked via a disulfide bond to form the mature receptor. Under normal conditions, c-MET dimerizes and autophosphorylates upon ligand binding, which in turn creates active docking sites for proteins that mediate downstream signaling leading to the activation/modulation of a variety of proteins. Such activation/modulation evokes a variety of pleiotropic biological responses leading to increased cell growth, scattering and motility, invasion, protection from apoptosis, branching morphogenesis, and angiogenesis. However, under pathological conditions improper activation of c-MET may confer proliferative, survival and invasive/metastatic abilities of cancer cells.

The mesenchymal epithelial transition (MET) kinase proto-oncogene has been know for almost 30 years, and MET kinase activity is dysregulated and/or upregulated in a range of cancers including, but not limited to, lung, breast, ovarian, kidney, colorectal, stomach and head and neck cancer. This receptor tyrosine kinase is activated by hepatic growth factor (HGF) but is also structurally related to the insulin growth factor receptor family. See, e.g., Lawrence and Salgia, MET molecular mechanisms and therapies in lung cancer. Cell Adhes. Migrat. 4(1):146-152 (2009); Jung, et al., Progress in cancer therapy targeting c-MET signaling pathway. Arch. Pharm. Res. 35:595-604 (2012). Met kinase is heterodimer and contains numerous cysteine residues that form disulfide bonds between the heterodimeric subunits. Like most receptor tyrosine kinases, MET kinase undergoes autophosphorylation and is coupled to a range of intracellular signaling pathways that regulate cell growth including, but not limited, to FAK, RAS, RAC, PI3K, CAS-CRK and other pathways. See, e.g., Eder, et al., Novel therapeutic inhibitors of the c-MET signaling pathway in cancer. Clin. Cancer Res. 15(7):2207-2214 (2012).

Molecular interactions between HGF and MET are important and are postulated to play an important role in cancer metastasis. See, e.g., Mizuno and Nakamura, HGF-MET cascade, a key target for inhibiting cancer metastasis: The impact of NK4 discovery on cancer biology and therapeutics. Int. J. Mol. Sci. 14:888-919 (2013). Importantly, MET kinase has been shown to be overexpressed in up to 40% of lung cancer tissue samples and has been a focal target for small molecule development. See, e.g., Villaflor and Salgia, Targeted agents in non-small cell lung cancer therapy: What is there on the horizon. J. Carcinog. 12:7-11 (2013). A subset of NSCLC patients have MET kinase amplification (upregulation) and this amplification is associated with resistance to the important NSCLC drugs Erlotinib and/or Gefitinib. See, e.g., Bean J, Brennan C, Shih J Y, et al., MET amplification occurs with or without T790M mutations in EGFR mutant lung tumors with acquired resistance to gefitinib or erlotinib. Proc. Natl. Acad. Sci. U.S.A. 26; 104(52):20932-20937 (2007). Additionally, a range of mutations in MET kinase are also associated with NSCLC, and dual dysregulation or aberrant expression of MET kinase receptors and EGFR has been specifically noted in lung cancer. See, e.g., Lawrence R E, Salgia R. MET molecular mechanisms and therapies in lung cancer. Cell Adhes. Migr. 4(1):146-152 (2010). MET kinase is coupled to FAK, RAS, RAC, PI3K, CAS-CRK and other pathways. These pathways are central to cell growth and also regulate various physiological processes in cancer (invasion, metastasis, and the like). The extracellular region possess the following characteristics: (i) a region of homology to semaphorins (Sema domain), which includes the full α-chain and the N-terminal part of the β-chain; (ii) a cysteine-rich MET-related sequence (MRS domain); (iii) glycine-proline-rich repeats (G-P repeats); and (iv) four immunoglobulin-like structures (Ig domains), a typical protein-protein interaction region. The intercellular, juxtamembrane region possesses the following characteristics: (a) a serine residue (Ser 985), which inhibits the receptor kinase activity upon phosphorylation; (b) a tyrosine (Tyr 1003), which is responsible for c-MET polyubiquitination, endocytosis, and degradation upon interaction with the ubiquitin ligase CBL; (c) a tyrosine kinase domain, which mediates c-MET biological activity (following c-MET activation, transphosphorylation occurs on Tyr 1234 and Tyr 1235); and (d) a C-terminal region contains two crucial tyrosines (Tyr 1349 and Tyr 1356), which are inserted into the multisubstrate docking site, capable of recruiting downstream adapter proteins with Src homology (SH2) domains. The two tyrosines of the docking site have been reported to be necessary and sufficient for the signal transduction both in vitro. See, generally, Trusolino, L., Bertotti, A. and Comoglio, P. M., MET signaling: principles and functions in development, organ regeneration and cancer. Nat. Rev. Mol. Cell Biol. 11:834-848 (2010).

### c-MET Activation

c-MET activation by its ligand HGF induces c-MET kinase catalytic activity, which triggers transphosphorylation of the tyrosines—Tyr¹²³⁴ and Tyr¹²³⁵. These two tyrosines engage various signal transducers, thus initiating a whole spectrum of biological activities driven by MET, collectively known as the invasive growth program. The transducers interact with the intracellular multisubstrate docking site of c-MET either directly, such as GRB2, SHC, SRC, and the p85 regulatory subunit of phosphatidylinositol-3 kinase (PI3K), or indirectly through the scaffolding protein Gab1. Tyr¹³⁴⁹ and Tyr¹³⁵⁶ of the multisubstrate docking site are both involved in the interaction with GAB1, SRC, and SHC, while only Tyr¹³⁵⁶ is involved in the recruitment of GRB2, phospholipase Cγ (PLC-γ), p85, and SHP2. GAB1 is a key coordinator of the cellular responses to MET and binds the MET intracellular region with high avidity, but low affinity. Upon interaction with MET, GAB1 becomes phosphorylated on several tyrosine residues which, in turn, recruit a number of signaling effectors, including PI3K, SHP2, and PLC-γ. GAB1 phosphorylation by MET results in a sustained signal that mediates most of the downstream signaling pathways. See, e.g., Marshall, C. J. Specificity of receptor tyrosine kinase signaling: transient versus sustained extracellular signal-regulated kinase activation. Cell 80(2):179-185 (1995). Such activation evokes a variety of pleiotropic biological responses leading to increased cell growth, scattering and motility, invasion, protection from apoptosis, branching morphogenesis, and angiogenesis. However, under pathological conditions improper activation of c-MET may confer proliferative, survival and invasive/metastatic abilities of cancer cells. See, e.g., Trusolino, L., Bertotti, A. and Comoglio, P. M., MET signalling: principles and functions in development, organ regeneration and cancer. Nat. Rev. Mol. Cell Biol. 11:834-848 (2010).

### Activation of Signal Transduction

c-MET engagement activates multiple signal transduction pathways including: (i) the RAS pathway mediates HGF-induced scattering and proliferation signals, which lead to branching morphogenesis. HGF is different from most mitogens in that it induces sustained RAS activation, and thus prolonged MAPK activity; (ii) the phosphatidylinositol 3-kinase (PI3K) pathway is activated in two ways—PI3K can be either downstream of RAS, or it can be recruited directly through the multifunctional docking site. Activation of the PI3K pathway is currently associated with cell motility through remodeling of adhesion to the extracellular matrix as well as localized recruitment of transducers involved in cytoskeletal reorganization, such as RAC1 and PAK. PI3K activation also triggers a survival signal due to activation of the AKT pathway; (iii) the STAT pathway, together with the sustained MAPK activation, is necessary for the HGF-induced branching morphogenesis. MET activates the STAT 3 transcription factor directly, through an SH2 domain: (iv) the β-catenin pathway, a key component of the Wnt signaling pathway, translocates into the nucleus following MET activation and participates in transcriptional regulation of numerous genes; and (v) the Notch pathway, through transcriptional activation of Delta ligand (DLL3).

### Role in Development

MET mediates a complex program known as invasive growth. Activation of c-MET triggers mitogenesis and morphogenesis. During embryonic development, transformation of the flat, two-layer germinal disc into a three-dimensional body depends on transition of some cells from an epithelial phenotype to spindle-shaped cells with motile behavior (i.e., a mesenchymal phenotype). This process is referred to as epithelial-mesnenchymal phenotype (EMT). Later in embryonic development, MET is crucial for gastrulation, angeogenesis, myoblast migration, bone remodeling, and nerve sprouting, embryogenesis, among others. See, e.g., Birchmeier C, Gherardi, E. Developmental roles of HGF/SF and its receptor, the c-Met tyrosine kinase. Trends Cell Biol. 8(10):404-410 (1998). Furthermore, c-MET is required for such critical processes as hepatic regeneration and wound healing during adulthood.

### MET Gene Mutation and Amplification

As mentioned, somatic mutations on the MET gene are rarely found in patients with nonhereditary cancer. To date, missense mutations and single nucleotide polymorphisms (SNPs) have been found in the SEMA and juxtamembrane domain of MET, whereas, activating mutations have been described mainly in NSCLC, hereditary and spontaneous renal carcinomas, hepatocellular carcinomas, gliomas, gastric, squamous cell carcinoma of the head and neck, and breast carcinomas. See, e.g., Stella, G. M., Benvenut, S., et al. MET mutations in cancers of unknown primary origin (CUPs). Hum. Mutat. 32:44-50 (2011). Potentially oncogenic mutations primarily involve point mutations that generate an alternative splicing encoding a shorter protein that lacks exon 14 (which encodes for the juxtamembrane domain of c-MET). Point mutations in the kinase domain render the enzyme constitutively active; whereas Y1003 mutations that inactivate the Cb1 binding site lead to constitutive c-MET expression. See, e.g., Ma, P. C., Tretiakova, M. S., et al. Expression and mutational analysis of MET in human solid cancers. Genes Chromosomes Cancer 47:1025-1037 (2008). In contrast, several other point mutations (e.g., N375S, R988C and T1010I) have been reported as SNPs, as they have been shown to lack transforming abilities. See, e.g. John, T., Kohler, D., et al. The ability to form primary tumor xenografts is predictive of increased risk of disease recurrence in early-stage non-small cell lung cancer. Clin. Cancer Res. 17:134-141(2011).

The most frequent genetic alteration is gene amplification, and as a consequence high c-MET protein expression and activation which has been reported as associated with a poor prognosis in non-small cell lung carcinoma (NSCLC), colorectal and gastric cancers. There were also reports that MET is more frequently amplified in metastatic tumors, suggesting a role in the late phases of malignant progression. See, e.g., Go, H., Jeon, Y. K., et al. High MET gene copy number leads to shorter survival in patients with nonsmall cell lung cancer. J. Thorac. Oncol. 5:305-313 (2010).

### c-MET Protein Overexpression

Over the years many groups have established that c-MET and HGF are highly expressed in a large number of solid and soft tumors (for a comprehensive list, see www.vai.org/met). The list of tumors in which c-MET is expressed is quite large, and it has been shown that high levels of c-MET can lead to the constitutive activation of the enzyme, as well as rendering cells sensitive to subthreshold amounts of HGF. Although many of these studies have not identified the level of c-MET receptor activity/phosphorylation or compared the expression level with its normal counterpart, it could be speculated that it is expressed with autocrine loops of HGF/c-MET which increase cell proliferation and metastases. See, e.g., Navab, R., Liu, J., et al. Co-overexpression of Met and hepatocyte growth factor promotes systemic metastasis in NCI-H460 non-small cell lung carcinoma cells. Neoplasia 11:1292-1300 (2009). Furthermore, independent studies have also shown that HGF is expressed ubiquitously throughout the body, showing this growth factor to be a systemically available cytokine as well as coming from the tumor stroma. See, e.g., Vuononvirta, R., Sebire, N.J., et al. Expression of hepatocyte growth factor and its receptor met in Wilms' tumors and nephrogenic rests reflects their roles in kidney development. Clin. Cancer Res. 15:2723-2730 (2009). A positive paracrine and autocrine loop of c-MET activation can therefore lead to further MET expression.

### Possible Functions of c-MET in Cancer

c-MET was first identified as the product of a chromosomal rearrangement after treatment with the carcinogen N-methyl-NO-nitro-N-nitrosoguanidine, See, e.g., Cooper, C. S., Park, M., et al., Molecular cloning of a new transforming gene from a chemically transformed human cell line. Nature 311:29-33 (1984). This rearrangement results in a constitutively fused oncogene (TPR-MET) which is translated into an oncoprotein following dimerization by a leucine-zipper motif located in the TPR moiety. This provides the structural requirement for c-MET kinase to be constitutively active. TPR-MET has been shown to possess the ability to transform epithelial cells and to induce spontaneous mammary tumors when ubiquitously over-expressed in transgenic mice. These findings set the starting point for a currently ongoing effort to unveil all oncogenic abilities of c-MET. It took more than a decade to provide the proof of concept for the role of c-MET in human cancers, which became evident following the identification of activating point mutations in the germline of patients affected by hereditary papillary renal carcinomas. See, e.g., Schmidt, L., Junker, K., et al., Novel mutations of the MET proto-oncogene in papillary renal carcinomas. Oncogene 18:2343-2350 (1999). A large number of reports have shown that an altered level of RTK activation can play an important role in the pathophysiology of cancer. See, e.g., Lemmon, M. A. and Schlessinger, J. Cell signaling by receptor tyrosine kinases. Cell 141:1117-1134 (2010). Deregulation and the consequent aberrant signaling of c-MET may occur by different mechanisms including gene amplification, abnormal expression, activating mutations, increased autocrine or paracrine ligand-mediated stimulation, and interaction with other active cell-surface receptors.

Many studies have reported that c-MET is overexpressed in a variety of carcinomas including lung, breast, ovary, kidney, colon, thyroid, liver, and gastric carcinomas. See, e.g., Knowles, L. M., Stabile, L. P., et al. HGF and c-Met participate in paracrine tumorigenic pathways in head and neck squamous cell cancer. Clin. Cancer Res. 15:3740-3750 (2009). Such over-expression could be the result of transcriptional activation, hypoxia-induced over-expression, or as a result of MET amplification. See, e.g., Cappuzzo, F., Marchetti, A., et al. Increased MET gene copy number negatively affects survival of surgically resected non-small-cell lung cancer patients. J. Clin. Oncol. 27:1667-1674 (2009); Cappuzzo, F., Janne, P. A., et al. MET increased gene copy number and primary resistance to gefitinib therapy in non-small-cell lung cancer patients. Ann. Oncol. 20:298-304 (2009). In addition, transgenic mice overexpressing c-MET have been reported to spontaneously develop hepatocellular carcinoma, and when the transgene was inactivated, tumor regression was reported even in large tumors. See, e.g., Wang, R., Ferrell, L. D., et al. Activation of the Met receptor by cell attachment induces and sustains hepatocellular carcinomas in transgenic mice. J. Cell. Biol. 153:1023-1034 (2001).

Abnormal MET activation in cancer correlates with poor prognosis, where aberrantly active MET triggers tumor growth, formation of new blood vessels (angeogenesis) that supply the tumor with nutrients, and cancer spread to other organs (metastasis). MET is deregulated in many types of human malignancies, including cancers of the: kidney, liver, stomach, breast, and brain. Normally, only stem cells and progenitor cells express MET, which allows these cells to grow invasively in order to generate new tissues in an embryo or regenerate damaged tissues in an adult. However, cancer stem cells are thought to hijack the ability of normal stem cells to express MET, and thus become the cause of cancer persistence and spread to other sites in the body.

Molecular interactions between HGF and MET are important and are postulated to play an important role in cancer metastasis. See, e.g., Mizuno and Nakamura, HGF-MET cascade, a key target for inhibiting cancer metastasis: The impact of NK4 discovery on cancer biology and therapeutics. Int. J. Mol. Sci. 14:888-919 (2013). Importantly, MET kinase has been shown to be overexpressed in up to 40% of lung cancer tissue samples and has been a focal target for small molecule development. See, e.g., Villaflor and Salgia, Targeted agents in non-small cell lung cancer therapy: What is there on the horizon. J. Carcinog. 12:7-11 (2013). A subset of NSCLC patients have MET kinase amplification (upregulation) and this amplification is associated with resistance to the important NSCLC drugs Erlotinib and/or Gefitinib. See, e.g., Bean J, Brennan C, Shih J Y, et al., MET amplification occurs with or without T790M mutations in EGFR mutant lung tumors with acquired resistance to gefitinib or erlotinib. Proc. Natl. Acad. Sci. U.S.A. 26; 104(52):20932-20937 (2007). Additionally, a range of mutations in MET kinase are also associated with NSCLC, and dual dysregulation or aberrant expression of MET kinase receptors and EGFR has been specifically noted in lung cancer. See, e.g., Lawrence R E, Salgia R. MET molecular mechanisms and therapies in lung cancer. Cell Adhes. Migr. 4(1):146-152 (2010). MET kinase is coupled to FAK, RAS, RAC, PI3K, CAS-CRK and other pathways. These pathways are central to cell growth and also regulate various physiological processes in cancer (invasion, metastasis, and the like).

### MET Kinase Experimental Methodologies and Results

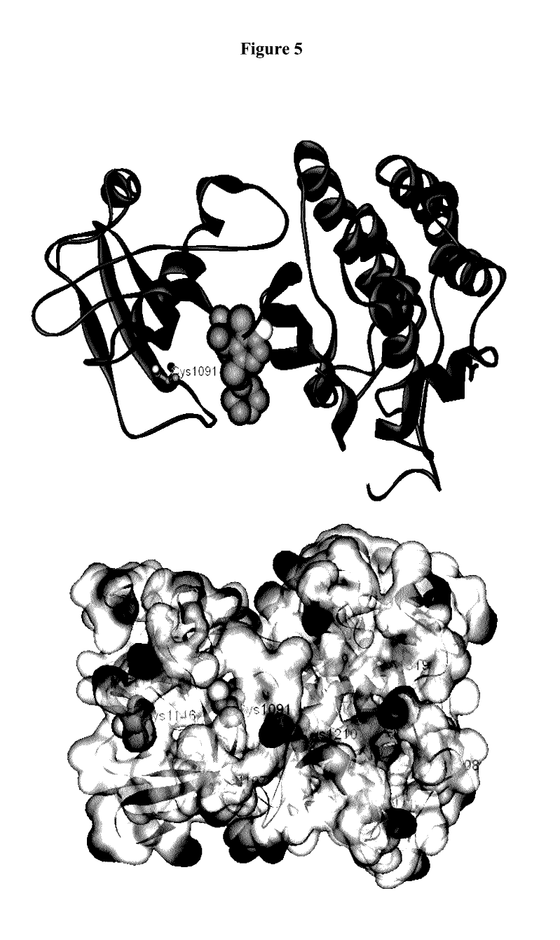

Computational analyses led to the hypothesis that Tavocept might interact with and modify human mesenchymal epithelial transition (MET) kinase. Studies in the specific example of MET kinase described herein were designed to evaluate the effect of Tavocept on MET kinase activity in the presence and absence of the known ATP-competitive MET kinase inhibitor, Crizotinib and Staurosporine. Specifically, it was hypothesized that Tavocept may xenobiotically modify Cys1091 from the Phosphate-loop (P-loop). Since the P-loop is located on top of the ATP substrate binding site, Tavocept-mediated xenobiotic modification at this site may impact MET kinase activity. Other modifications may be possible and an X-ray structure would be needed to unequivocally verify this hypothesis. See, FIG. 5.

I. Materials and Methods

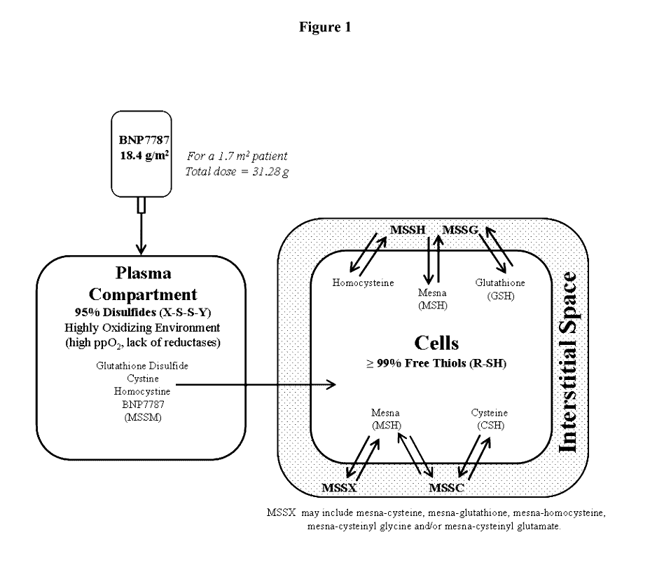

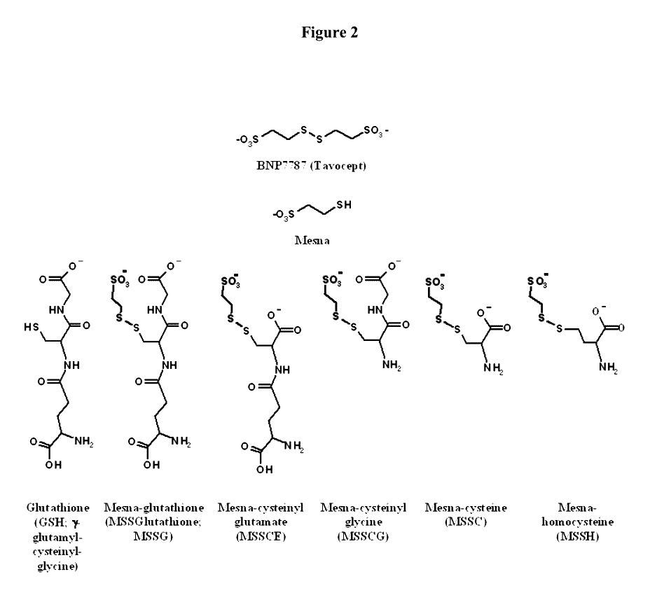

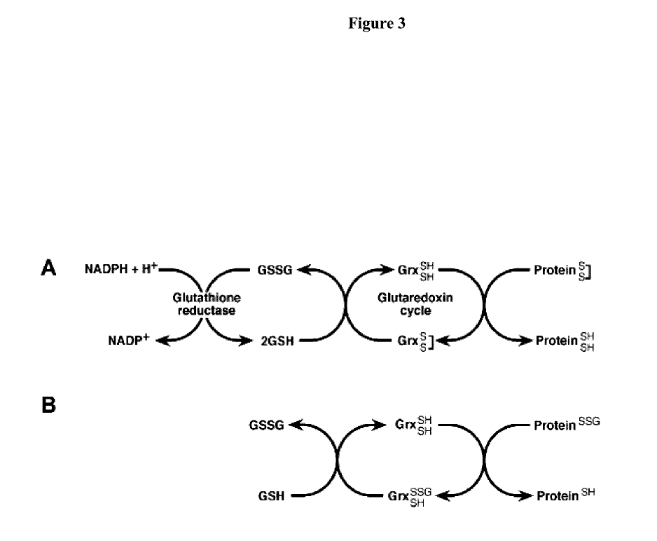

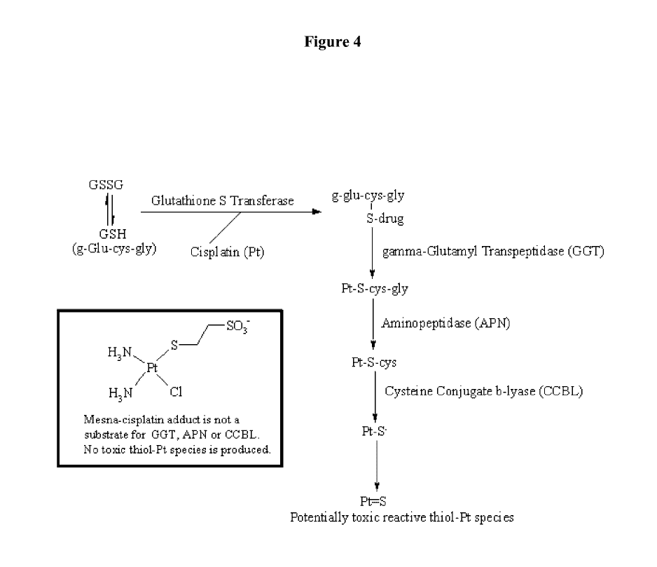

N-terminal GST tagged recombinant human MET expressed in Sf9 cells was purchased from SignalChem (FIG. 1; Cat. #M52-18G-10, lots V273-2, MW 81.0 kDa and aliquoted to 10 μL fractions when it was used for the first time (so as to avoid multiple freeze/thaw cycles for subsequent experiments). Tavocept (BNP7787) was prepared by a proprietary method (lots #205001 or 450002-2, >97%, no mesna was detected by Mass Spectroscopy). Kinase inhibitor, PF-02341066 (also known as Crizotinib), was purchased from Selleck Chemicals, LLC (Cat. #877399-52-5, lot S1068802). Kinase inhibitor, Staurosporine, was purchased from Calbiochem, LLC (Cat. #569396, lot #D00127851). The substrate that was phosphorylated by the kinase, polyglutamate-tyrosine (PolyGT), was purchased from SignalChem (Cat. #P61-58, lot #R098-4).

### Structure of the MET ATP Competitive Inhibitor—Crizotinib

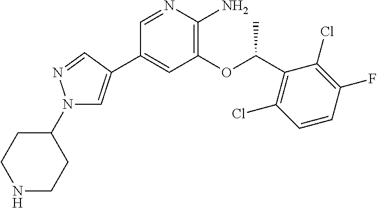 [SMILES: C[C@@H](Oc1cc(-c2cnn(C3CCNCC3)c2)cnc1N)c1c(Cl)ccc(F)c1Cl]

### Structure of the MET ATP Competitive Inhibitor—Staurosporine

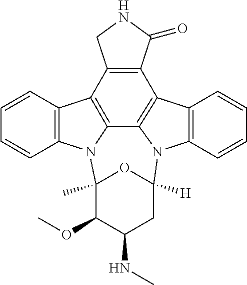 [SMILES: CN[C@@H]1C[C@H]2O[C@@](C)([C@@H]1OC)n1c3ccccc3c3c4c(c5c6ccccc6n2c5c31)C(=O)NC4]

Kinase assay buffer was purchased from SignalChem (Cat. # K03-09, Lot # R301-3W) and consisted of 40 mM Tris, pH 7.5, 20 mM MgCl₂, 0.1 mg/mL bovine serum albumin (BSA) and 150 μM dithiothreitol (DTT). Microplates were purchased directly from VWR or Corning and initial assay optimization was performed using whole area 96-well white microplates (Corning 3912, lot 29011050) but to save reagents and costs, most IC₅₀ determinations and subsequent experiments were conducted in half area 96 well white microplates (Corning 3642, lot 05312045).

ADP-glo reagents were purchased from Promega and consisted of ADP (V916A, lot 32551702), ATP (V915A, lot 32559501), ADP-glo (V912A, lot 32559601 or V912B, lot 0000010953), kinase detection reagent buffer (V913A, lot 32179101 or V913B, lot 0000010953) and kinase detection substrate (V914A, lot 30286301 or V914B, lot 0000010722). All other reagents were purchased from Sigma Aldrich. A Tecan Ultra microplate reader with XFluor4 software (Tecan, V4.51) and RdrOle software (Tecan, V4.50) were used in this study.

II. MET Kinase Assay

Assays quantitated ADP produced in reactions where MET was incubated with ATP, polyGT substrate, buffer and varying concentrations of Tavocept (BNP7787), Crizotinib, Staurosporine, or a combination thereof, using the ADP-Glo system by Promega. MET phosphorylated the polyGT substrate using the ATP cofactor and produced ADP. Initially, 25 μL volumes were used for assays; subsequently half-area 96-well microplates were obtained that allowed reduction of the assay volume to 10 μL, thereby significantly reducing reagent consumption. The 10 μL volume assays in half area 96-well microtiter plates contained MET (2.5 ng/μL) with ATP (100 μM) or MET (0.1 ng/μL) with ATP (10 μM), PolyGT substrate (0.1 μg/μL) and the concentrations of Tavocept (BNP7787)) and/or Crizotinib and/or Staurosporine as indicated (final assay volume was 10 mL). For most assays, a stock of ATP (1 mM) and PolyGT (1 mg/mL) was used. Crizotinib and Staurosporine was dissolved as a 5 mM and 1 mM stock in DMSO, respectively, and then further diluted in kinase assay buffer (DMSO only controls were always run to ensure that DMSO did not interfere with the assay). The reactions, in microplate, were incubated for 60 minutes at 25° C. on a heat block. Following this 60 minute incubation, the kinase activity was evaluated using the ADP-Glo system from Promega that monitored ADP produced when MET phosphorylated the PolyGT substrate.

III. ADP-Glo Detection

Kinase assays were run in triplicate or quadruplicate in microplates. Following this, the ADP-Glo detection system (Promega) was used to determine how much ADP had been produced. For 10 μL volume assays, to each microplate well containing 10 μL of kinase reaction was added ADP-Glo reagent (10 μL), plates were spun in a table top centrifuge (1000 rpm (123×g) for 1 minute) to ensure no reagent remained on the well walls, and then agitated for 1 minute to ensure optimal mixing. Plates were incubated at 25 C on a heat block for 40 minutes. Next, kinase detection reagent (20 μL) was added and, as above, centrifugation and agitation were repeated; plates were allowed to incubate at 25° C. on a heat block for 30 minutes. Following the incubation of the kinase detection reagent, plates were read on a Tecan Ultra microplate reader. The Tecan Ultra contained a built-in plate definition file for the whole area 96-well white Corning plates but a plate definition file for the half area 96-well Corning plates was created using the RdrOle component of the Tecan Ultra software.

VI. Evaluation of MET Activity In Vitro and Determination of Assay Conditions

Kinases vary in their ability to turnover ATP in vitro; therefore, the activity of the MET over a concentration range from 0.78 ng to 20 ng was evaluated in assay volumes of 10 μL. See, FIG. 7. These values represent a molar range of 0.96 to 247 nM MET. Subsequently, it was decided to use: (i) a “high” MET concentration (2.5 ng/μL per assay) evaluated with “high” ATP concentration (100 μM per assay) and (ii) a “low” MET concentration evaluated (0.1 ng/μL per assay) with “low” ATP concentration (10 μM per assay).

Polyglutamate tyrosine (polyGT; 4:1 ratio) was used as the substrate for phosphorylation and had an average polymer mass ranging from 20,000 to 50,000 g/mole; each glu-glu-glu-glu-tyr “subpolymer’ in this polymer has a mass of approximately 698 g/mole. Therefore, each mole of polymer of 20,000 g/mole would contain approximately 28 moles of the glu-glu-glu-glu-tyr “subpolymer” in a 10 μL assay volume containing 1 μg of the polyGT substrate. Assuming the lower polymer mass of 20,000 g/mol mass, this translates to approximately 5 μM polyGT per assay and 140 μM glu-glu-glu-glu-tyr “subpolymer” per assay. This was a vast excess of possible tyrosine phosphorylation sites, ensuring that the substrate for phosphorylation was not rate limiting (assuming the higher mass range would produce an even larger excess of glu-glu-glu-glu-tyr “subpolymers”).

V. Specific Experimental Results

Data from MET assays run on the Tecan Ultra microplate spectrophotometer were collected in Microsoft Excel. Error calculations and graphical representations were performed in Microsoft Office Excel (Microsoft Corporation, Redmond, Wash., USA). Determination of IC₂₅ and IC₅₀ values were accomplished using Origin Lab software (OriginLab Corporation, Northampton, Mass., USA).

A. Tavocept Inhibits MET Kinase Activity In Vitro

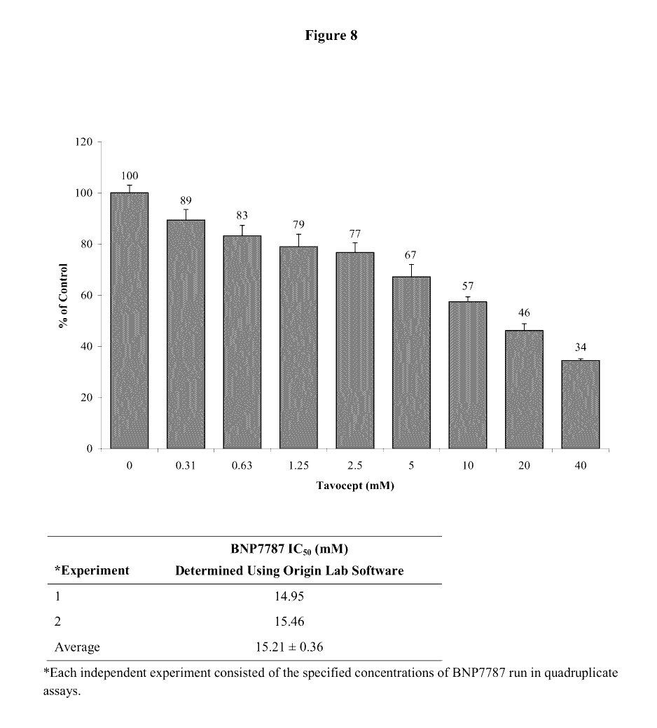

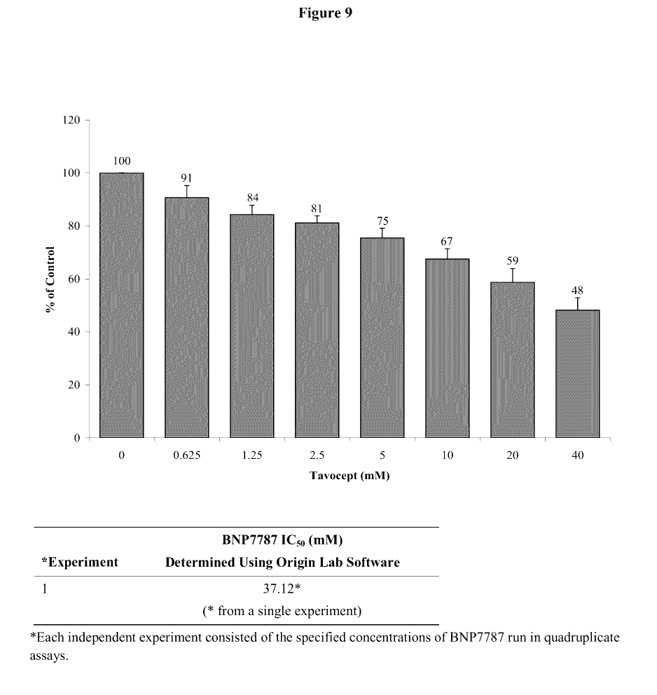

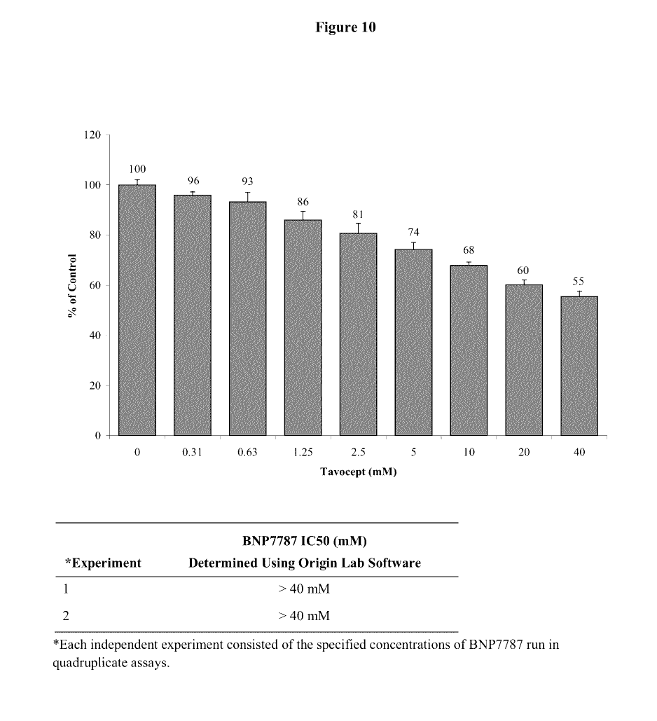

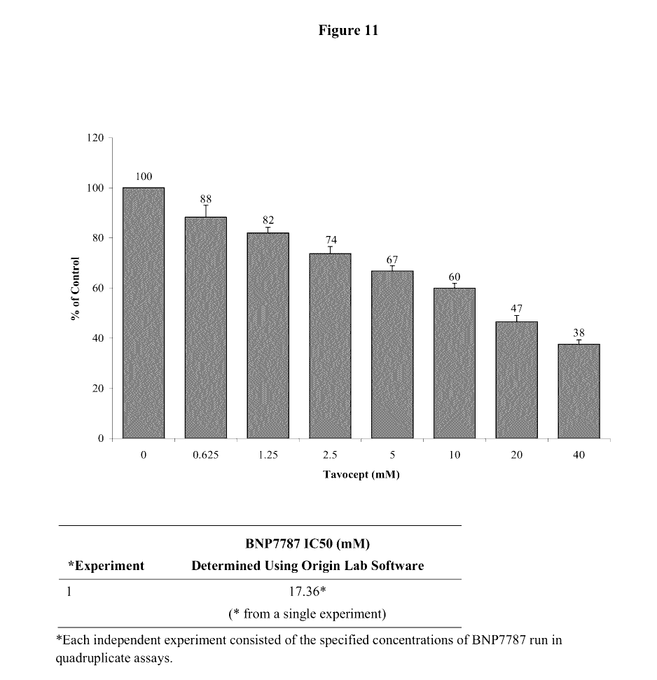

Tavocept (BNP7787) inhibited MET with an IC₅₀ of 17.36 mM (single experiment) under assay conditions of 10 μM ATP (with 2.5 ng/μL MET per assay) and with an IC₅₀ of 15.21±0.36 mM under assay conditions of 10 μM ATP (with 0.1 ng/μL MET per assay). At higher ATP (100 μM), Tavocept (BNP7787) had an IC₅₀>40 mM and 37.12 mM (single experiment), respectively, in assays containing 2.5 or 0.1 ng/μL MET per assay, respectively. See, FIG. 8, FIG. 9, FIG. 10, and FIG. 11.

These varying ATP concentrations were used in an effort to see if Tavocept (BNP7787) had either a competitive or non-competitive inhibitory effect, with respect to ATP binding, on MET. Typically, in kinase endpoint assays like the Promega ADP-Glo assay system, inhibitors are classified as competitive if their IC₅₀ increases notably as the ATP concentration increases. It was observed in the studies disclosed herein that as the ATP concentration was increased, the IC₅₀ for Tavocept (BNP7787) also increased. Table 6, below, illustrates the IC₅₀ of Tavocept (BNP7787) under varying concentrations of MET and ATP. Consequently, while the inhibition of MET by Tavocept (BNP7787) is not “classic” competitive inhibition (i.e., where ATP and Tavocept (BNP7787) have nearly identical or at least significantly overlapping binding sites and only one molecule, either ATP or Tavocept (BNP7787), can occupy that site at a time), it is “competitive-like” based upon the increasing IC₅₀ as the ATP concentration is increased.

TABLE 6
IC₅₀ of Tavocept under varying concentrations of MET and ATP

| MET (ng/μL) | ATP (μM) | IC₅₀ |
|---|---|---|
| 2.5 | 10 | 17.36* |
| 2.5 | 100 | >40 |
| 0.1 | 10 | 15.21 ± 0.36 |
| 0.1 | 100 | 37.12* |

*IC₅₀ values without errors are from single experiments.

Physiologically, concentrations of Tavocept (BNP7787) as high as 18 mM have been achieved in the clinic. Tavocept (BNP7787) has been administered at a dose of 18.4 g/m² and this translates to Cₘₐₓ values in plasma of 10 mM and higher. The concentration of Tavocept (BNP7787) required to see an effect in vitro on MET activity under the lower ATP assay conditions (10 μM) are physiologically relevant. The concentration of Tavocept (BNP7787) required to observe an effect on MET activity under the higher ATP assay conditions (100 μM) are not physiologically relevant. However, when Tavocept (BNP7787) is used in combination with Crizotinib or staurosporine, notable potentiation occurs under both lower and higher ATP assay conditions.

ATP is often in the millimolar range in vivo, and the human body is reported to contain no more than 0.5 moles (˜250 g) of ATP at any time, but this supply is constantly and efficiently recycled. See, e.g., Lu X, Errington J, Chen V J, Curtin N J, Boddy A V, Newell D R. Cellular ATP depletion by LY309887 as a predictor of growth inhibition in human tumor cell lines. Clin. Cancer Res. 6(1):271-277 (2000). In vivo there are many ATP-dependent enzymes that compete for ATP binding, including kinases, synthetases, helicases, membrane transporters and pumps, chaperones, motor proteins, and large protein complexes like the proteasome; therefore, the concentrations of 10 and 100 μM ATP used herein are approximations for ATP concentrations that may be available to MET in vivo as it competes for ATP with various other enzymes and proteins that utilize ATP.

B. Crizotinib Inhibits MET In Vitro

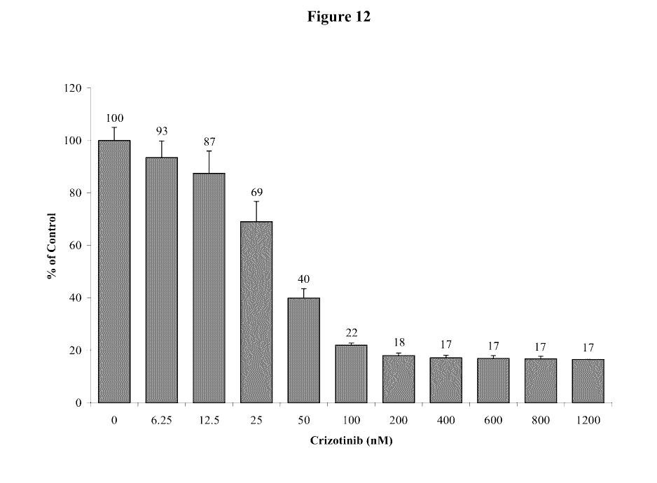

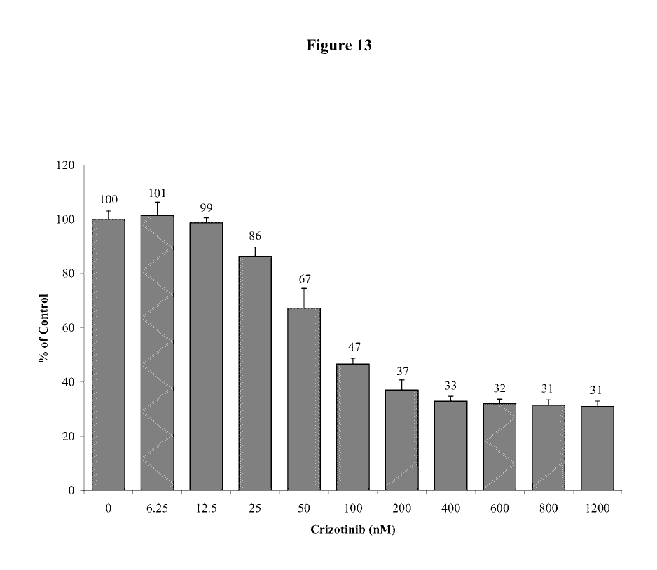

Crizotinib is a reported ATP-competitive inhibitor of MET. See, e.g., Bang Y J. The potential for Crizotinib in non-small cell lung cancer: a perspective review. Ther. Adv. Med. Oncol. 3(6):279-291 (2011). In the in vitro kinase studies reported herein, we observed that Crizotinib inhibited MET with an IC₅₀ of 38.39 nM (see, FIG. 12) under assay concentrations using ATP at 10 μM and with an IC₅₀ of 87.8 nM (see, FIG. 13) under assay concentrations using ATP at 100 μM. As mentioned above, Crizotinib has previously been characterized as a competitive inhibitor of MET, with respect to ATP, and our data are consistent with this previously reported observation. It should be noted that in clinical trials where Crizotinib was administered orally at doses of 250 mg twice daily, concentrations of Crizotinib of 57 nM were reported.

(i) Tavocept Potentiates the Inhibitory Effect of Crizotinib on MET (0.1 ng/μL) Activity In Vitro Under 10 μM ATP Conditions

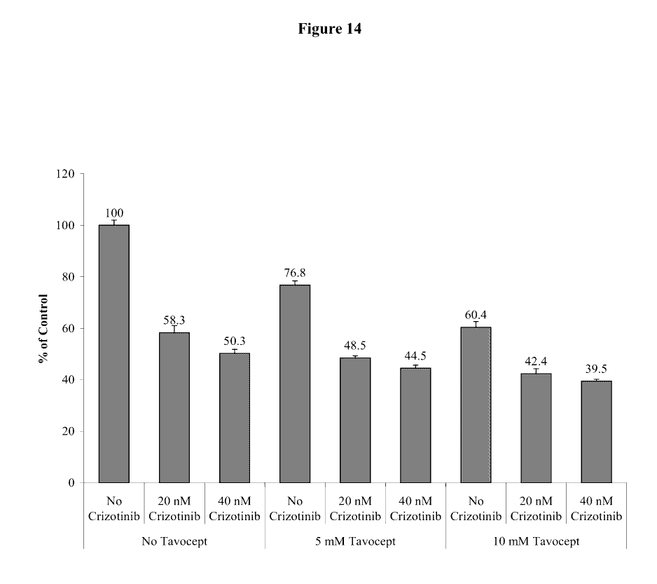

Under assay conditions with 10 μM ATP, 5 mM Tavocept (BNP7787) in combination with 20 nM Crizotinib (near the IC₂₅ value for Crizotinib) resulted in 10% greater inhibition than 20 nM Crizotinib alone; 10 mM Tavocept (BNP7787) in combination with 20 nM Crizotinib resulted in 16% greater inhibition than 20 nM Crizotinib alone. Under assay conditions with 10 μM ATP, 5 mM Tavocept (BNP7787) in combination with 40 nM Crizotinib (near the IC₅₀ value of Crizotinib) resulted in 6% greater inhibition than 40 nM Crizotinib alone whereas 10 mM Tavocept (BNP7787) in combination with 40 nM Crizotinib resulted in 11% greater inhibition than 40 nM Crizotinib alone. These assays near the IC₅₀ value for Crizotinib (i.e., 40 nM, when ATP is 10 μM), have similar stimulation compared to 10 μM ATP and 20 nM Crizotinib conditions (see, FIG. 14). As discussed previously (see, FIG. 9-FIG. 13), Tavocept (BNP7787) alone or Crizotinib alone were both effective at inhibiting MET in vitro.

(ii) Tavocept Potentiates the Inhibitory Effect of Crizotinib on MET (2.5 ng/μL) Activity In Vitro Under 100 μM ATP Conditions

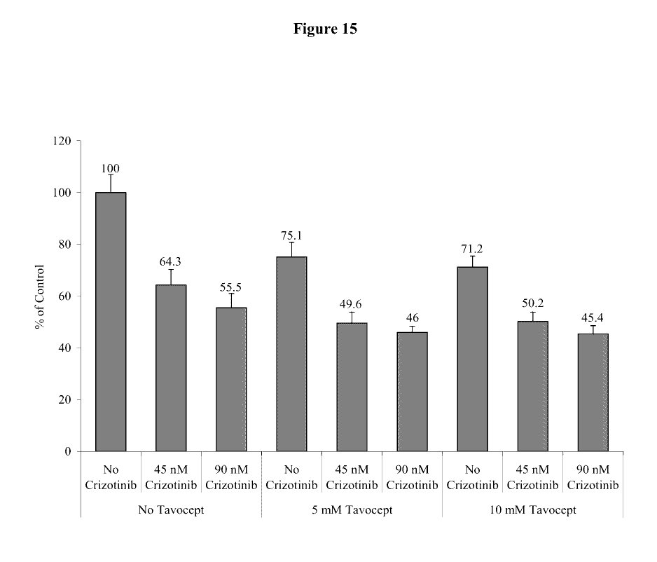

The effect of physiologically achievable concentrations of Tavocept (BNP7787) near the IC₂₅ and IC₅₀ concentrations of Crizotinib under assay conditions with either 100 μM or 10 μM ATP were examined. Concentrations of Crizotinib of 57 nM have been reported in clinical trials; therefore, concentrations of Crizotinib used in these studies are within physiologically relevant ranges. Currently, Tavocept (BNP7787) is administered at a dose of 18.4 g/m² and this translates to Cₘₐₓ values in plasma of 10 mM and higher. Tavocept (BNP7787) notably potentiates the inhibitory effect of Crizotinib on MET at physiologically relevant concentrations of both Tavocept (BNP7787) and Crizotinib. Under assay conditions with 100 μM ATP, 5 mM Tavocept (BNP7787) in combination with 45 nM Crizotinib (near the IC₂₅ value for Crizotinib) resulted in 15% greater inhibition than 45 nM Crizotinib alone; whereas 10 mM Tavocept (BNP7787) in combination with 45 nM Crizotinib resulted in 14% greater inhibition than 45 nM Crizotinib alone. Under assay conditions with 100 μM ATP, 5 mM Tavocept (BNP7787) in combination with 90 nM Crizotinib (near the IC₅₀ value of Crizotinib) resulted in 10% greater inhibition than 90 nM Crizotinib alone; whereas 10 mM Tavocept (BNP7787) in combination with 90 nM Crizotinib resulted in 10% greater inhibition than 90 nM Crizotinib alone. These assays near the IC₅₀ value for Crizotinib (i.e., 90 nM, when ATP is 100 μM), have similar stimulation compared to 100 μM ATP and 45 nM Crizotinib conditions. See, FIG. 15. In addition, as discussed in preceding sections, Tavocept (BNP7787) alone or Crizotinib alone or Tavocept (BNP7787) in combination with Crizotinib were effective at inhibiting MET in vitro.

C. Staurosporine Inhibits MET In Vitro

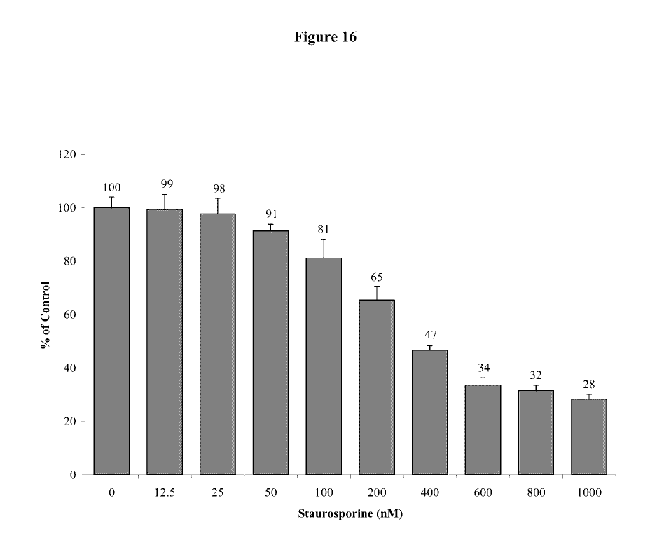

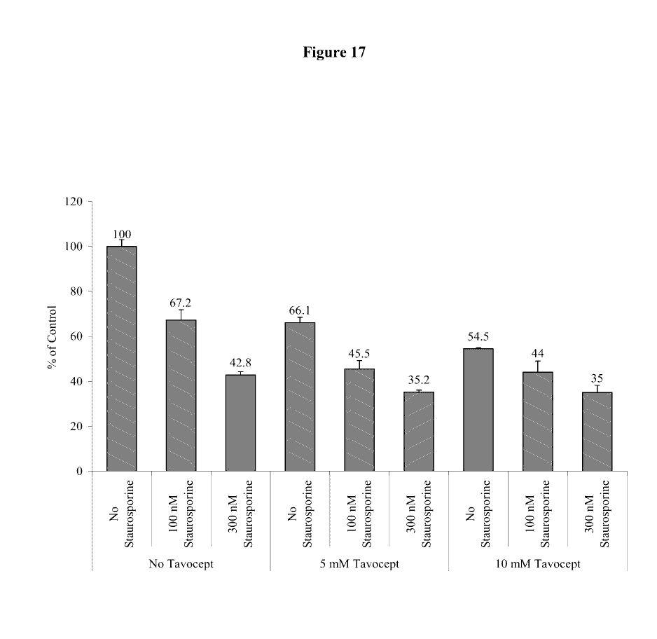

Staurosporine is a reported ATP-competitive inhibitor of many kinases. See, e.g., Tanramlu D, Schreyer A, Pitt W R, Blundell T L. On the origins of enzyme inhibitor selectivity and promiscuity: a case study of protein kinases binding to staurosporine. Chem. Biol. Drug Des. 74(1):16-24 (2009). In the in vitro kinase studies reported herein, we observed that Staurosporine inhibited MET (0.1 ng/μL) with an IC₅₀ of 340 nM (see, FIG. 16) under assay concentrations using ATP at 10 μM.

(i) Tavocept Potentiates the Inhibitory Effect of Staurosporine on MET (0.1 ng/μL) Activity In Vitro Under 10 μM ATP Conditions

The effect of physiologically achievable concentrations of Tavocept (BNP7787) near the IC₂₅ and IC₅₀ concentrations of Staurosporine under assay conditions with 10 μM ATP were evaluated. Currently, Tavocept (BNP7787) is administered at a dose of 18.4 g/m² and this translates to Cₘₐₓ values in plasma of 10 mM and higher. Tavocept (BNP7787) notably potentiates the inhibitory effect of Staurosporine on MET at the tested concentrations of both Tavocept (BNP7787) and Staurosporine. Under assay conditions with 10 μM ATP, 5 mM Tavocept (BNP7787) in combination with 100 nM Staurosporine resulted in 22% greater inhibition than 100 nM Staurosporine alone; whereas 10 mM Tavocept (BNP7787) in combination with 100 nM Staurosporine resulted in 23% greater inhibition than 100 nM Staurosporine alone. See, FIG. 17. Under assay conditions with 10 μM ATP, 5 mM or 10 mM Tavocept (BNP7787) in combination with 300 nM Staurosporine (i.e., near the IC₅₀ value of Staurosporine), respectively, resulted in 8% and 8% greater inhibition than 300 nM Staurosporine alone. In addition, as discussed in preceding sections, Tavocept (BNP7787) alone or Staurosporine alone both were effective at inhibiting MET in vitro.

Experiments where combinations of Tavocept (BNP7787) and Staurosporine were evaluated together under high ATP conditions (100 μM) were not pursued, as the IC₅₀ concentration of Staurosporine alone on MET kinase under assay conditions with 100 μM ATP is >800 nM.

VI. Experimental Results Conclusions

The results from these experimental studies support the following conclusions:
In assays with 100 μM ATP, Tavocept (BNP7787) inhibits MET (2.5 ng/μL) with an IC₅₀ value >40 mM.
In assays with 10 μM ATP, Tavocept (BNP7787) inhibits MET (0.1 ng/μL) with an IC₅₀ value of 15.21±0.36 mM.
In assays with 100 μM ATP, Crizotinib inhibits MET (2.5 ng/μL) with an IC₅₀ value of 87.8 nM (single experiment).
In assays with 10 μM ATP, Crizotinib inhibits MET (0.1 ng/μL) with an IC₅₀ value of 38.39 nM (single experiment).
Tavocept (BNP7787) and Crizotinib together, inhibit MET kinase more than either test article alone.
In assays with 100 μM ATP (MET=2.5 ng/μL) and 45 nM Crizotinib, 5 and 10 mM Tavocept (BNP7787), respectively, resulted in 14% and 15%, greater inhibition than 45 nM Crizotinib alone.
In assays with 100 μM ATP (MET=2.5 ng/μL) and 90 nM Crizotinib, 5 and 10 mM Tavocept (BNP7787), respectively, resulted in 10% and 10%, greater inhibition than 90 nM Crizotinib alone.
In assays with 10 μM ATP (MET=0.1 ng/μL) and 20 nM Crizotinib, 5 and 10 mM Tavocept (BNP7787), respectively, resulted in 10% and 16% greater inhibition than 20 nM Crizotinib alone.
In assays with 10 μM ATP (MET=0.1 ng/μL) and 40 nM Crizotinib, 5 and 10 mM Tavocept (BNP7787), respectively, resulted in 6% and 11% greater inhibition than 40 nM Crizotinib alone.
In assays with 10 μM ATP, Staurosporine inhibits MET (0.1 ng/μL) with an IC₅₀ value of 340 nM.
In assays with 10 μM ATP (MET=0.1 ng/μL) and 100 nM Staurosporine, 5 and 10 mM Tavocept (BNP7787), respectively, resulted in 22% and 23% greater inhibition than 100 nM Staurosporine alone.
In assays with 10 μM ATP (MET=0.1 ng/μL) and 300 nM Staurosporine, 5 and 10 mM Tavocept (BNP7787), respectively, resulted in 8% and 8% greater inhibition than 300 nM Staurosporine alone.
Tavocept modulates the activity of MET kinase in vitro, if this occurs in vivo, a potential survival benefit could accompany this MET kinase modulation in NSCLC patients bearing MET kinase fusions or mutations.

(ii) Anaplastic Lymphoma Kinase (ALK)

Anaplastic lymphoma kinase (ALK) also known as ALK tyrosine kinase receptor or CD246 (cluster of differentiation 246) is an enzyme that in humans is encoded by the ALK gene. See, e.g., Cui, J. J.; Tran-Dubé, M.; et al., Structure Based Drug Design of Crizotinib (PF-02341066), a Potent and Selective Dual Inhibitor of Mesenchymal-Epithelial Transition Factor (c-MET) Kinase and Anaplastic Lymphoma Kinase (ALK). J. Med. Chem. 54:6342-6363 (2011). ALK belongs to the family of insulin growth factor receptor kinases and fusions of ALK with other genes are common in several diseases and cancers. See, e.g., Palmer R H, Vernersson E, Grabbe C, Hallberg B. Anaplastic lymphoma kinase: Signalling in development and disease. Biochem. J. 420(3):345-361 (2009); Kruczynski, et al., Anaplastic lymphoma kinase as a therapeutic target. Expert Opin. Ther. Targets 16:1127-1138 (2012). Fusions in ALK result in constitutively active protein that results in stimulation of a variety of intracellular pathways critical for cell growth and proliferation. See, e.g., Webb, et al., Anaplastic lymphoma kinase: Role in cancer pathogenesis and small-molecule inhibitor development for therapy. Expert Rev. Anticancer Ther. 9(3):331-356 (2009). At least seven different variants of ALK fusions with the gene encoding the echinoderm microtubule-associated protein-like 4 (EML4) are known to occur in NSCLC. See, Id. Additionally, fusions between the tropomyosin receptor kinase fused gene (TFG) and ALK (TFG-ALK) are also known to occur in NSCLC. See, e.g., Hernandez L, Pinyol M, Hernandez S, Bea S, Pulford K, Rosenwald A, et al. TRK-fused gene (TFG) is a new partner of ALK in anaplastic large cell lymphoma producing two structurally different TFG-ALK translocations. Blood 94(9):3265-3268 (1999). EML4-ALK fusions are thought to account for approximately 2-7% of NSCLC cases. See, e.g., Palmer R H, Vernersson E, Grabbe C, Hallberg B. Anaplastic lymphoma kinase: signalling in development and disease. Biochem. J. 420(3):345-361 (2009); Heuckmann, et al., Differential protein stability and ALK inhibitor sensitivity of EML4-ALK fusion variants. Clin. Cancer Res. 18:4682-4690 (2012). While many of the studies disclosed herein involve non-small cell lung cancer (NSCLC), ALK fusions (including the nucleophosmin-ALK (NPM-ALK)) fusion, are found in a range of other cancers including, but not limited to, breast cancer, colorectal cancer, esophageal cancer, anaplastic large cell lymphoma, chronic myelogenous leukemia, and acute leukemias. See, e.g., Grande, et al., Targeting oncogenic ALK: A promising strategy for cancer treatment. Mol. Cancer Ther. 10:569-579 (2011); Ok, et al., Aberrant activation of the hedgehog signaling pathway in malignant hematological neoplasms. Am. J. Path. 180:2-11 (2012).

ALK is coupled to numerous signaling pathways that regulate cell proliferation including Ras-ERK, JAK3-STAT3, PLCγ and PI3K and, therefore, represents an important target for anti-cancer drug development. See, e.g., Chiarle, R. et al., The anaplastic lymphoma kinase in the pathogenesis of cancer. Nat. Rev. Cancer 8(1):11-23 (2008); Ou, Crizotinib: A novel and first-in-class multitargeted tyrosine kinase inhibitor for the treatment of anaplastic lymphoma kinase rearranged non-small cell lung cancer and beyond. Drug Design, Devel. Therap. 4:471-485 (2011). ALK provides us with one of the most recognized examples of personalized medicine success in Crizotinib, which effectively modulates ALK function in NSCLC patients harboring ALK fusions despite being initially developed to target MET kinase. See, e.g., Ong, et al., Personalized medicine and pharmacogenetic biomarkers: Progress in molecular oncology testing, Expert Rev. Mol. Diagnosis 12(6):593-602 (2012).

Much remains to be learned about ALK. For example, it is not clear if the known ALK ligands, pleiotrophin and midkine, are the sole ALK ligands in vivo or if other ligands exist (these molecules activate other receptors and are certainly not exclusive to ALK). Additionally, while ALK fusions are known to be important in a number of cancers, point mutations in ALK resulting in gain-of-function mutants are also known and are associated with increases in ALK kinase activity, ALK-mediated phosphorylation of downstream targets, and ALK expression levels. See, e.g., See, e.g., Grande, et al., Targeting oncogenic ALK: A promising strategy for cancer treatment. Mol. Cancer Ther. 10:569-579 (2011); Palmer R H, Vernersson E, Grabbe C, Hallberg B. Anaplastic lymphoma kinase: Signalling in development and disease. Biochem. J. 420(3):345-361 (2009). ALK point mutations are also thought to be important in one of the leading causes of cancer deaths in children, neuroblastoma. See, e.g., Carpenter and Mosse, Targeting ALK in neuroblastoma: Preclinical and clinical advancements. Nat. Rev. Clin. Oncol. 9(7):391-399 (2012). ALK represents an important target for anti-cancer drug development across a range of cancers and agents that modulate ALK, as single agents or in combination with other ALK agents, may have widespread clinical utility.

### ALK Receptor Structure and Function

ALK belongs to the tyrosine kinase receptor family. By homology, ALK is most similar to leukocyte tyrosine kinase, and both belong to the insulin-receptor superfamily. ALK is a single-chain transmembrane receptor comprising three structural domains. The extracellular domain contains an N-terminal signal peptide sequence and is the ligand-binding site for the putative activating ligands of ALK (i.e., pleiotrophin and midkine) This is followed by the transmembrane and juxtamembrane region which contains a binding site for phosphotyrosine-dependent interaction with insulin receptor substrate-1. The final section has an intracellular tyrosine kinase domain with three phosphorylation sites (Y1278, Y1282, and Y1283), followed by the C-terminal domain with interaction sites for phospholipase C-γ and Src homology 2 domain containing SHC. These sequences are absent in the product of the transforming ALK gene. Under physiologic conditions, binding of a ligand induces homodimerization of ALK, leading to trans-phosphorylation and kinase activation. In ALK translocations, the 5′-terminus fusion partners provide dimerization domains, enabling ligand-independent activation of the kinase. In addition, unlike native ALK, which localizes to the plasma membrane, the majority of ALK fusion proteins localize to the cytoplasm. This difference in cellular localization may also contribute to deregulated ALK activation.

The EML4-ALK fusion oncogene represents one of the newest molecular targets in cancer (especially in non-small cell lung carcinoma (NSCLC)). EML4-ALK was identified by the screening of a cDNA library derived from a the tumor of a NSCLC (adenocarcinoma) of the lung. See, e.g., Soda, M., Choi, Y. L, et al. Identification of the transforming EML4-ALK fusion gene in non-small cell lung cancer. Nature 448:561-566 (2007). This fusion arises from an inversion on the short arm of chromosome 2 [Inv (2) (p21p23)] that joins exons 1-13 of echinoderm microtubule associated protein-like 4 (EML4) to exons 20-29 of ALK. The resulting chimeric protein, EML4-ALK, contains an N-terminus derived from EML4 and a C-terminus containing the entire intracellular tyrosine kinase domain of ALK. Since the initial discovery of this fusion, multiple other variants of EML-ALK have been reported, all of which encode the same cytoplasmic portion of ALK but contain different truncations of EML4. See, e.g., Choi, Y. L., Takeuchi, K., et al. Identification of novel isoforms of the EML4-ALK transforming gene in non-small cell lung cancer. Cancer Res. 68:4971-4976 (2008). In addition, fusions of ALK with other partners including TRK-fused gene (TFG) and KIF5B have also been described in lung cancer, but seem to be much less common than EML4-ALK. See, e.g., Rikova, K., Guo, A., et al. Global survey of phosphotyrosine signaling identifies oncogenic kinases in lung cancer. Cell 131:1190-1203 (2007).

Chromosomal aberrations involving ALK have been identified in several other cancers, including anaplastic large cell lymphomas (ALCL), inflammatory myofibroblastic tumors (IMT), and neuroblastomas. See, e.g., Chiarle, R., Voena, C., et al. The anaplastic lymphoma kinase in the pathogenesis of cancer. Nat. Rev. Cancer 8:11-23 (2008). In cases of ALK translocation, including EML4-ALK, the fusion partner has been shown to mediate ligand-independent dimerization of ALK, resulting in constitutive kinase activity. In cell culture systems, EML4-ALK possesses potent oncogenic activity. In transgenic mouse models, lung-specific expression of EML4-ALK leads to the development of numerous lung adenocarcinomas. See, e.g., Soda, M., Takada, S., et al. A mouse model for EML4-ALK-positive lung cancer. Proc. Natl. Acad. Sci. U.S.A. 105:19893-19897 (2008). Cancer cell lines harboring the EML4-ALK translocation can be effectively inhibited by small molecule inhibitors targeting ALK. See, e.g., Koivunen, J. P., Mermel, C., et al. EML4-ALK fusion gene and efficacy of an ALK kinase inhibitor in lung cancer. Clin. Cancer Res. 14:4275-4283 (2008). Treatment of EML4-ALK transgenic mice with ALK inhibitors also results in tumor regression. Taken together, these aforementioned results support the notion that ALK-driven lung cancers are dependent upon the fusion oncogene.

### ALK Pathways

In mammals, the precise function of ALK has yet to be elucidated. See, e.g., Palmer R. H., Vernersson, E., Grabbe, C., Hallbergm B. Anaplastic lymphoma kinase: signalling in development and disease. Biochem. J. 420:345-361 (2009). On the basis of its expression pattern in the mouse, ALK is believed to play a role in the development and function of the nervous system. Studies using ALK knockout mice have reported an increase in hippocampal progenitor proliferation and an increase in dopamine levels within the basal cortex. See, e.g., Bilsland, J. G., Wheeldon, A., et al. Behavioral and neurochemical alterations in mice deficient in anaplastic lymphoma kinase suggest therapeutic potential for psychiatric indications. Neuropsychopharmacology 33:685-700 (2008). In contrast, in the adult, ALK expression is restricted primarily to the central and peripheral nervous systems.

Although the ligand for ALK is known in Drosophila melanogaster, no homolog of this ligand has been identified in vertebrates. Putative ALK ligands include pleiotrophin (PTN) and midkine, both of which are small, heparin-binding growth factors, implicated in neuron development as well as neurodegenerative diseases. See, e.g., Palmer, R. H., Vernersson, E., Grabbe, C., Hallberg, B. Anaplastic lymphoma kinase: signalling in development and disease. Biochem. J. 420:345-361 (2009). Pleiotrophin and midkine have a similar distribution to ALK, mainly in the nervous system during fetal development followed by downregulation at birth. These ligands display neurotrophic functions on receptor binding.

Additional experimental results have also suggested that PTN may also activate ALK indirectly by binding to and inactivating the receptor protein tyrosine phosphatase Z1. See, e.g., Perez-Pinera P., Zhang, W., et al. Anaplastic lymphoma kinase is activated through the pleiotrophin/receptor protein-tyrosine phosphatase beta/zeta signaling pathway: an alternative mechanism of receptor tyrosine kinase activation. J. Biol. Chem. 282:28683-28690 (2007). Whether there are other ALK ligands or other mechanisms of ALK activation remains to be determined.

The key downstream effectors of ALK are better understood than the upstream activators. The oncogenic fusion protein promotes the activation of, primarily, three key signaling pathways: (i) the Janus-activated kinase (JAK3)-STAT3 intracellular pathway; (ii) phosphoinositide 3-kinase (PI3K)—Akt pathway; and (iii) the Ras/mitogen activated protein/extracellular signal regulated kinase (ERK) kinase (Mek)/Erk pathway to promote cell cycle progression, survival, and proliferation. See, e.g., Mosse, Y. P., Wood, A., Maris, J. M Inhibition of ALK signaling for cancer therapy. Clin. Cancer Res. 15:5609-5615 (2009). Activation of the phospholipase C-g is also thought to contribute to NPM-ALK-mediated transformation. STAT3 seems to be the key player in survival mechanisms promoted by ALK in ALCL, its inhibition having been shown to prevent NPM-ALK-induced transformation in vivo. In addition, sonic hedgehog (SHH) signaling has been shown to be activated in ALKp ALCL through PI3K-AKT, because of the amplification of SHH, and this may also be involved in cell cycle progression and survival. See, e.g., Singh, R. R., Cho-Vega, J. H., et al. Sonic hedgehog signaling pathway is activated in ALK-positive anaplastic large cell lymphoma. Cancer Res. 69:2550-2558 (2009). NIPA, a SCF-type E3 ligase, has been cloned in a complex with NPM-ALK (see, e.g., Ouyang, T., Bai, R. Y., et al. Identification and characterization of a nuclear interacting partner of anaplastic lymphoma kinase (NIPA). J. Biol. Chem. 278:30028-30028 (2004)) and has been suggested to be involved in NPM-ALK-mediated cell cycle progression. NPM-ALK promotes inactivation of NIPA, which prevents cyclin B1 degradation, therefore, allowing cell cycle progression. See, e.g., Bassermann, F., von Klitzing, C., et al. NIPA defines an SCF-type mammalian E3 ligase that regulates mitotic entry. Cell 122:45-57 (2005). Although the downstream intracellular signaling pathways and other oncogenic ALK mutants and fusion proteins have not been fully elucidated, STAT3 signaling seems to play a key role in the pathogenesis of EML4-ALK tumors. See, e.g., Mano, H. Non-solid oncogenes in solid tumors: EML4-ALK fusion genes in lung cancer. Cancer Sci. 99:2349-2355 (2008).

These pathways have been most extensively studied in the context of ALCL- and NPM-ALK-mediated transformation. In general, the Ras/Mek/Erk pathway is important for driving cell proliferation; whereas the PI3K/Akt and JAK3-STAT3 pathways are important for cell survival and cytoskeletal changes. Although different ALK fusions may differentially activate downstream signaling pathways, EML4-ALK, like NPM-ALK, signals through Erk and PI3K. Pharmacologic inhibition of EML4-ALK using TKIs leads to downregulation of Ras/Mek/Erk and PI3K/Akt and apoptosis, consistent with the notion that activation of these two pathways is critical for EML4-ALK-mediated transformation. See, e.g., Koivunen, J. P., Mermel, C., et al. EML4-ALK fusion gene and efficacy of an ALK kinase inhibitor in lung cancer. Clin. Cancer Res. 14:4275-4283 (2008). Furthermore, in models of acquired ALK TKI resistance, both Ras/Mek/Erk and PI3K/Akt pathways are reactivated despite the continued presence of the TKI.

### ALK Translocations in Cancer

The best characterized alterations of ALK associated with cancer are gene rearrangements; these have been observed in hematologic as well as in non-hematologic malignancies. The role of ALK in cancer was first identified as part of the NPM-ALK gene fusion involved in the pathogenesis of a subset of anaplastic large cell lymphoma (ALCL; see, e.g., Li, S. Anaplastic lymphoma kinase-positive large B-cell lymphoma: a distinct clinicopathological entity. Int. J. Clin. Exp. Pathol. 2:508-518 (2009)). Subsequently, multiple fusion partners forming ALK chimeric proteins in this disease have also been identified. ALK rearrangements have also been reported in other lymphomas, such as diffuse large B-cell lymphomas (DLBCL). In solid tumors, ALK translocations were first described in inflammatory myofibroblastic tumors (IMT).

As previously described, the novel fusion transcript with transforming activity, formed by the translocation of echinoderm microtubule associated protein like 4 (EML4) located at 2p21, and the ALK located at 2p23, has been described in a subset of patients with non-small cell lung cancer (NSCLC; see, e.g., Soda, M., Choi, Y. L, et al. Identification of the transforming EML4-ALK fusion gene in non-small cell lung cancer. Nature 448:561-566 (2007)) and several additional variants of the rearranged gene have also been identified. Furthermore, other ALK partners, such as the kinesin family member 5B (KIF5B) located at 10p11.22 and TRK fused gene (TFG) located at 3q12.2, have been described in NSCLC, and cases with atypical translocation (i.e., loss of centromeric probe, as assessed by FISH) with an unknown partner have also been identified in lung cancer samples. See, e.g., Salido, M., Pijuan, L., et al. Increased ALK gene copy number and amplification are frequent in non-small cell lung cancer. J. Thorac. Oncol. 6:21-27 (2011).

In other solid tumors, such as esophageal squamous cell carcinoma, colorectal cancer, and breast cancer, ALK alterations have been described, but their roles in the pathogenesis of these malignancies remain to be elucidated. See, e.g., Lin, E., Li, L., et al. Exon array profiling detects EML4-ALK fusion in breast, colorectal, and non-small cell lung cancers. Mol. Cancer Res. 7:1466-1477 (2009). These abnormal proteins consistently conserve the intracellular domain of ALK, whereas the partners retain the coiled-coil oligomerization domain. This biological property results in ligand-independent dimerization and, thus, in constitutive activation of the kinase. The oncogenic role of ALK chimeric proteins has also been shown by pre-clinical studies and mouse models with forced expression of ALK. Of note is that, in NSCLC, the ALK translocation seems to define a subgroup of patients with specific clinical, pathologic, and molecular characteristics. This alteration is more frequent in younger patients, who are non- or light-smokers, with adenocarcinoma histology with presence of signet ring-type cells, EGFR, and/or KRAS wildtype tumors (see, e.g., Wong, D. W., Leung, E. L., et al. University of Hong Kong Lung Cancer Study Group. The EML4-ALK fusion gene is involved in various histologic types of lung cancers from nonsmokers with wild-type EGFR and KRAS. Cancer 115:1723-1733 (2009)), together with non-c-MET copy number increase (see, e.g., Varella-Garcia, M., Cho, Y., Lu, X. ALK gene rearrangements in unselected caucasians with non-small cell lung carcinoma (NSCLC). J. Clin. Oncol. 28:75(Suppl):10533 (2010)); however, a number of cases not fitting into this subgroup have also been described (see, e.g., Martelli, M. P., Sozzi, G., et al. EML4-ALK rearrangement in non-small cell lung cancer and non-tumor lung tissues. Am. J. Pathol. 174:661-670 (2009)).

### ALK Point Mutations Mutations in Cancer

Point mutations have been found in 6-8% of primary neuroblastomas. Germ-line mutations have been identified in families with more than one sibling with neuroblastoma. Somatic mutations with wild-type ALK in matched constitutional DNAs have also been described in non-familial neuroblastoma cases. These mutations are located mainly in the TK domain; the most frequent being the gain-of-function mutations F1174L and R1275Q. These mutations are associated with increased expression, phosphorylation, and kinase activity of the ALK protein. Further, they have been shown to have Ba/F3 cell-transforming capacity. In some cases, these mutations coexist with an increased copy number of the ALK gene. See, e.g., Janoueix-Lerosey, I., Lequin, D., et al. Somatic and germline activating mutations of the ALK kinase receptor in neuroblastoma. Nature 455:967-970 (2008). Interestingly, these mutations (particularly the F1174L) are predictive of response (as indicated by increased apoptosis and inhibition of growth) to short hairpin ALK-specific knockdown and TK ALK inhibitors (TAE684 and PF-12341066). Notably, protein expression levels in ALK mutant neuroblastoma models do not directly correlate with sensitivity toALK inhibitors. It seems that this finding could be explained by the existence of a higher turnover rate of the ALK protein in cells with constitutively activated ALK.

### ALK Amplifications in Cancer

An increased copy number of ALK has also been described in neuroblastoma cell lines and tumors, which can coexist with ALK gene mutation. In this disease, amplification, as well as mutation of ALK, has been associated with MYCN amplification, the most frequent amplicon in neuroblastoma defining a high-risk subgroup of patients that may benefit from ALK-selective inhibition. See, e.g., Janoueix-Lerosey, I., Lequin, D., et al. Somatic and germline activating mutations of the ALK kinase receptor in neuroblastoma. Nature 455:967-970 (2008).

In addition, a number of research groups have described ALK gene amplification in non-small cell lung cancer (NSCLC) tissue. See, e.g., Perner, S., Wagner, P. L., et al. EML4-ALK fusion lung cancer. Neoplasia 10:298-302 (2008); Salido, M., Pijuan, L., et al. Increased ALK gene copy number and amplification are frequent in non-small cell lung cancer. J. Thorac. Oncol. 6:21-27 (2011); Grande, E., Bolos, M. V., Arriola, E. Targeting Oncogenic ALK: A Promising Strategy for Cancer Treatment. Mol. Cancer Ther. 10:569-579 (2011). A recent study showed a relatively high frequency of copy number of mainly low level gains (60%), and amplification (10%); wherein the pattern of amplification, in the majority of NSCLC cases, was found to be characterized by a small percentage of cells within the tumor harboring this amplification. See, Grande, E., Bolós, M. V., Arriola, E. Targeting Oncogenic ALK: A Promising Strategy for Cancer Treatment. Mol. Cancer Ther. 10:569-579 (2011). However, it was found that some cases had >40% of cells with ALK amplification. It should be noted, that this amplification (i.e., copy number gain) was not associated with protein expression in the series of patients utilized in the study.

### ALK Kinase X-Ray Crystallographic Analysis and Results

Computational analyses conducted by the Applicants of the present patent application using known structural information, prompted them to hypothesize that Tavocept might interact with and modify human anaplastic lymphoma kinase (ALK). The kinase domain of human ALK contains five (5) cysteine residues (Cys1156, Cys1182, Cys1235, Cys1255, and Cys11259), and studies in the specific example of ALK described herein were designed to evaluate the effect of Tavocept on wild-type ALK kinase activity in the presence and absence of the known ATP-competitive ALK inhibitor, Crizotinib (PF-02341066). Additionally, whole protein MS on human ALK indicated that multiple Tavocept-derived mesna adducts form on human ALK (i.e., Tavocept may xenobiotically modify ALK at 4 or more cysteine sites), and X-ray crystallographic studies characterized two of these Tavocept-derived xenobiotic modification sites on cysteine residues 1156 and 1235. See, e.g., Dalle-Donne I, Rossi R, Colombo G, Giustarini D, Milzani A. Protein S-glutathionylation: a regulatory device from bacteria to humans. Trends Biochem. Sci. 34(2):85-96 (2009). Based on the structural data, the location of the mesna group on cysteine 1156 (Cys1156) interferes with the position of phenylalanine 1127 (Phe1127) in the P loop and results in a partial obstruction of the ATP binding pocket (see, FIG. 1). Additionally, the Tavocept-derived mesna adduct on Cys1156 is in close proximity to the catalytically important activation loop (A-loop) of ALK. Given these structural observations, it was hypothesized that Tavocept might inhibit and/or otherwise modulate ALK activity. Such an effect, if it occurred in vivo, could be a mechanism for increasing patient survival in patients bearing ALK fusion. Accordingly, studies herein were designed to evaluate the effect of Tavocept on ALK activity in the presence and absence of the known ATP-competitive ALK inhibitor, Crizotinib.

### I. Cloning, Expression and Purification of the Kinase Domain of ALK for X-Ray Crystallographic Analyses

Wild-type human ALK, consisting of residues 1095-1410, was cloned into a proprietary vector containing a C-terminal 6×His tag. Isolated shuttle vector was transformed into DH10Bac cells. Colonies containing bacmid with transposed ALK DNA were picked and grown overnight at 37° C. Bacmid DNA was isolated by DNA isopropanol precipitation and re-suspended in 100 μl of sterile water. Bacmid DNA was allowed to re-suspend at room temperature for 1 hour prior to transfection. Recombinant bacmid DNA was expressed in SF9 cells at a multiplicity of infection (MOI) of 2 in a 48 hour infection at 27° C. The cells were harvest by centrifugation and stored at −80° C.

Recombinant protein was expressed in SF9 cells at a MOI of 2 in a 48 hour infection at 27° C. The cells were harvested by centrifugation and stored at −80° C. Purification of target protein was done using a two column system (Ni-NTA and size exclusion). The cell biomass was lysed by sonification in 50 mM Tris-HCl pH 7.8, 500 mM NaCl, 10% glycerol, 20 mM Imidazole, (Buffer A) plus Roche Complete Protease Inhibitor Tablets, and 20,000 units Benzonase. Target protein was extracted by binding Ni-NTA (Qiagen). Protein was eluted with 250-500 mM Imidazole pH 7.8. Peak fractions were pooled and aggregated protein was separated from monomeric protein via Size Exclusion (S200 16/60, GE Healthcare Lifesciences) in 50 mM Bicine pH 8.4, 150 mM NaCl, 5 mM DTT. Monomeric protein was concentrated to ˜19 mg/ml.

### II. Preparation of Tavocept-Derived Mesna Adduct on ALK Crystals

ALK (19 mg/mL) in 50 mM Bicine, pH 8.4, 150 mM NaCl, and 25 mM DTT was incubated at 4° C. overnight to fully reduce the protein. DTT was removed by exchanging 5-times in 50 mM Bicine, pH 8.4, 150 mM NaCl using ultrafiltration (10 kDa cutoff Centricon filters). Fully reduced ALK was incubated with 5 mM Tavocept and incubated at 4° C. overnight. Protein was submitted for mass spectrometry analysis to confirm the presence of at least one Tavocept-derived mesna adduct prior to initiation of protein crystallization experiments.

### III. Crystallization of ALK Containing a Tavocept-Derived Mesna Adduct

Mass spectrometry analysis of C-terminal 6×his tag ALK that had been incubated with Tavocept indicated two likely Tavocept-derived mesna adducts; however, crystals were not able to be obtained from these protein samples. As an alternative, apo-ALK crystals were soaked with Tavocept and this yielded crystals with two Tavocept-derived mesna adducts. Briefly, the crystal of C-terminal 6×his tag ALK was obtained by sitting drop/vapor diffusion method by mixing 2 μL at 17 mg/mL protein (50 mM Bicine pH 8.4, 150 mM NaCl, 5 mM DTT) with 2 μL of 0.1 M TRIS hydrochloride pH 8.5, 0.2 M Sodium acetate trihydrate, 30% (w/v) Polyethylene glycol 4000 at 20° C. Diffracting crystals appeared within 5-8 days. Before data collection, the crystals were soaked in 20 mM Tavocept overnight and transferred into a cryoprotectant solution made up of 20% ethylene glycol (v/v) in crystallization buffer, after which they were flash-frozen in liquid nitrogen for data collection. Crystals diffracted to 2.1 Å. The mass spectrometry analysis of ALK after reaction with Tavocept suggested two (2) Tavocept-derived mesna adducts consistent with the X-ray crystallographic structure.

### IV. Data Collection

Diffraction data were collected at the Advanced Light Source (ALS) (Berkeley, Calif.). Tavocept-derived mesna adducts were observed on Cys 1156 and Cys 1235. Data was processed using the program package MosFlm as part of the ccp4 program package. Image processing statistics with crystal characteristics and data collection statistics (outer shell statistics in parenthesis) are summarized in Table 7 (for final electron density maps for the Tavocept-derived mesna adducts, see below).

TABLE 7
Crystal characteristics and data collection statistics
(outer shell statistics in parenthesis)

| | |
|---|---|
| Unit cell (Å, °) | 51.535 57.157 104.216 90.000 90.000 90.000 |
| Space group | P2₁2₁2₁ |
| Resolution range (Å) | 46.20-2.10 (2.21-2.10) |
| No. of observations | 102983 |
| No. of unique reflections | 18384 |
| Redundancy | 5.6 (5.7) |
| Completeness (%) | 98.8 (97.5) |
| Mean I/sigma(I) | 10.1 (2.8) |
| R_{merge} | 0.135 (0.621) |

### V. Structure Solution and Refinement

Data was indexed, integrated, scaled and merged using the program Mosflm. The structure was solved by molecular replacement with Phaser using a monomer from the Protein Data Bank, our internal structure of apo-ALK, which is very close in structure to PDB entry 2XP2 (human ALK in complex with Crizotinib). The structure was consistent with one molecule in the crystal asymmetric unit. The protein model was iteratively refit and refined using MIFitⁱ (MIFit Open Source Project, 2010) and REFMAC5. See, Murshudov G N, Vagin A A, Dodson E J. Refinement of macromolecular structures by the maximum-likelihood method. Acta Crystallogr. D. Biol. Crystallogr. 53(3):240-255 (1997). The solved structure is supported by contiguous electron density for most of the molecule, landmark side chain density features matching the amino acid sequence including cysteines, absence of phi-psi violations and final R/Rfree values in the normal range. Residual density observed near Cys 1235 and Cys 1156 was modeled as Tavocept-derived mesna adducts. Final statistics are summarized in Table 8. It should be noted that a number of side-chain atoms and protein fragments were not refined. The missing fragments included Gly 1123-Gly 1128 Ser 1136-Pro1144, Arg 1214-Pro 1218 and Ser 1281-Arg 1284.

TABLE 8
Crystallographic data and refinement statistics

| | |
|---|---|
| Resolution range (Å) | 46.196-2.100 |
| No. of reflections | 18340 (17402 working set, 938 test set) |
| No. of protein chains | 1 (A) |
| Ligand id codes | UNK, EDO |
| No. of protein residues | 293 |
| No. of ligands | 5 |
| No. of waters | 159 |
| No. of atoms | 2452 |
| Mean B-factor | 21.256 |
| R_{work} | 0.1927 |
| R_{free} | 0.2448 |
| Rmsd bond lengths (Å) | 0.010 |
| Rmsd bond angles (°) | 1.196 |
| No. of disallowed φψ angles | 0 |

### VI. Crystal Structure of ALK Bearing Tavocept-Derived Mesna Moiety

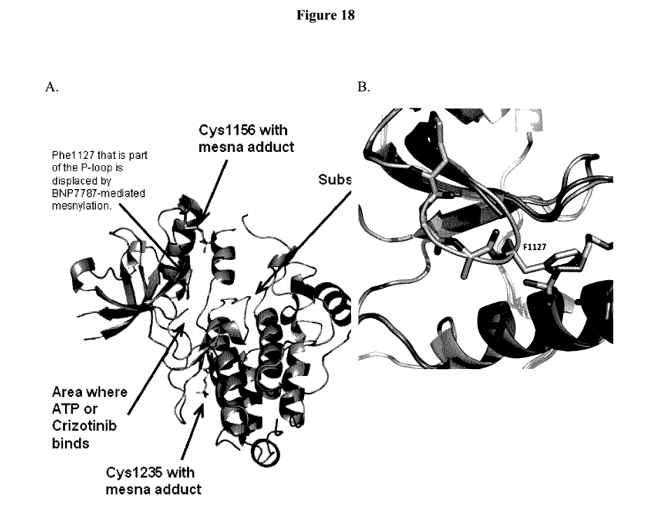

The crystal structure of ALK in complex with a Tavocept-derived mesna moiety was completed at 2.1 Å resolution. See, FIG. 18, Panel A. The protein crystallizes as a monomer in the asymmetric unit and Tavocept-derived mesna moieties were observed at Cys 1235 and Cys 1156. The adduct at Cys 1156 is located in close proximity to the active site and in fact results in substantial disorder of the so-named P-loop (or phosphate binding loop) which is highly conserved in protein kinases. This disorder prevented full refinement of the P-loop. While a large fragment of the P-loop is missing from the refined structure, comparison with the P-loop of the apo-ALK suggests that the Tavocept-derived mesna adduct at Cys 1156 interferes with the docking of Phe 1127 into a small pocket now occupied by mesna resulting in a destabilization of the loop's binding orientation. See, FIG. 18, Panel B.

### VII. Ligand Binding Site

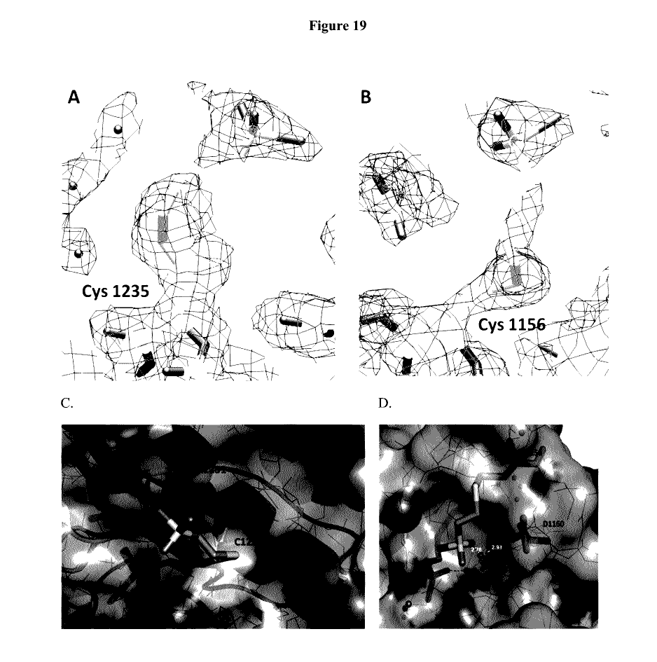

Close up views of the electron density map at the sites of the Tavocept-derived xenobiotically modified ALK cysteine residues is presented in FIG. 19, Panels A and B. For both of the Tavocept-derived mesna adducts at Cys 1235 and Cys 1156, a single conformation was observed. Both ligand binding sites are relatively solvent exposed. See, FIG. 19, Panels C and D. The Tavocept-derived mesna adduct at Cys 1235 is located in the “back” of the kinase domain relative to the position of the active site. Mesna is not interacting with any residues other than Cys 1235 although the sulfonate group is in close proximity to Arg 1231. See, FIG. 19, Panel D.

At the Cys 1156 site, the mesna sulfonate group makes a water-mediated hydrogen bond with the carbonyl of Asp 1160 (FIG. 19, Panel D). A fragment of the P-loop (Gly 1123-Gly 1128), another nearby fragment (Ser 1281-Arg 1284) and a number of residue side-chains are not in the final refined structure and interactions between a Tavocept-derived mesna adduct and these missing atoms cannot be ruled out. Additionally, interactions between a Tavocept-derived mesna adduct and Arg 1120 of another protein monomer in the crystal may also be a possibility.

This current structure of ALK with Tavocept-derived mesna adducts at Cys1156 and Cys1235 does not have the density for Tyr1282, Tyr1283, and Lys1285 which are part of the ALK activation-loop (A-loop); it is not clear if the loss of density for these residues is due to the presence of a Tavocept-derived mesna adduct on Cys1156 near this A-loop. However, as a point of reference, these residues are also disordered in the apo-ALK structure (data not shown) suggesting that this is an area with inherent disorder.

### ALK Kinase Experimental Methodologies and Results

### I. Materials and Methods

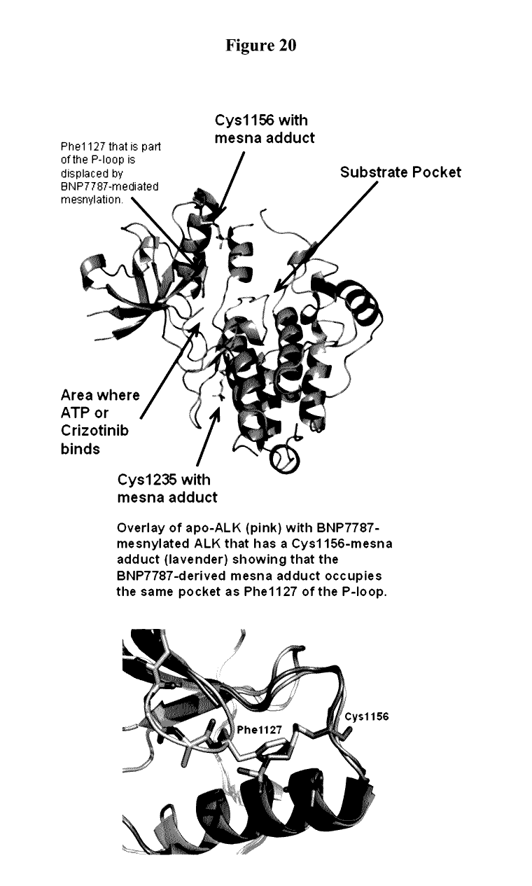

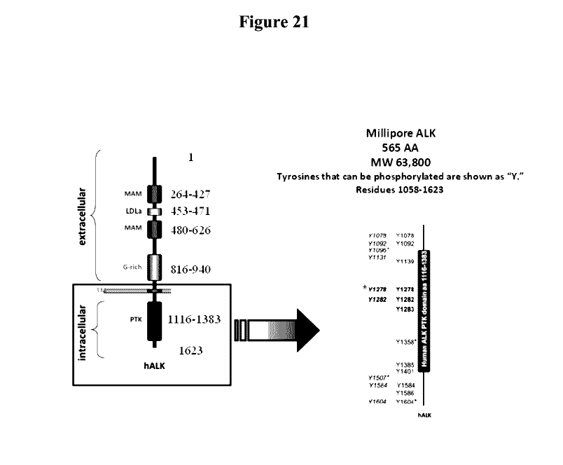

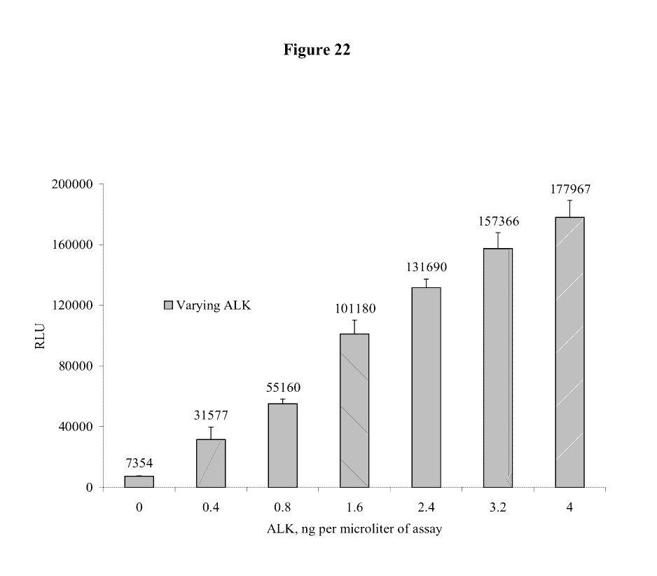

N-terminal 6×His tagged recombinant human ALK expressed in baculovirus Sf21 was purchased from Millipore (FIG. 20; lot 32944U-H, MW 63.8 kDa) and aliquoted to 1 μL fractions when it was used the first time (to avoid multiple freeze/thaws cycles for subsequent experiments). Tavocept was prepared by a proprietary method (lots #205001 or 450002-2, >97%, no mesna was detected by mass spectroscopy). Kinase inhibitor, PF-02340166 (also known as Crizotinib), was purchased from Selleck Chemicals, LLC (Cat. No. 877399-52-5, lot S1068802).

The substrate that was phosphorylated by the kinase, polyglutamate-tyrosine (PolyGT), was purchased from Sigma (P0275, lot 120M5007V).

### Structure of the ALK ATP Competitive Inhibitor, Crizotinib

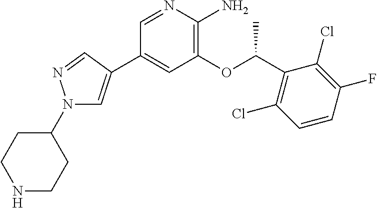 [SMILES: C[C@@H](Oc1cc(-c2cnn(C3CCNCC3)c2)cnc1N)c1c(Cl)ccc(F)c1Cl]

Kinase assay buffer was prepared and consisted of 20 mM HEPES (Sigma H-0891, lot 48H5432), 0.1% Brij 96 (aka Brij 35P, Sigma Aldrich 16005-2506-F, lot BCB07465V), 10 mM NaF (Sigma, 51504, lot 129H1425), 1 mM Na₃VO₄ (Sigma, S-6508, lot 061M0104), and 10 mM MnCl₂ (Sigma, M-5005, lot 108H0150) adjusted to a final pH of 7.5. Microplates were purchased directly from Corning and initial assay optimization was performed using whole area 96-well white microplates (Corning 3912, lot 29011050); however to save reagents and costs, most IC₅₀ determinations and subsequent experiments were conducted in half area 96-well white microplates (Corning 3642, lot 05312045).

ADP-glo reagents were purchased from Promega and consisted of ADP (V916A, lot 32551702), ATP (V915A, lot 32559501), ADP-glo (V912A, lot 32559601 or V912B, lot 0000010953), kinase detection reagent buffer (V913A, lot 32179101 or V913B, lot 0000010953) and kinase detection substrate (V914A, lot 30286301 or V914B, lot 0000010722). All other reagents were purchased from Sigma Aldrich. A Tecan Ultra microplate reader with XFluor software (Tecan, V4.51) and RdrOle software (Tecan, V4.50) were used in this study.

### II. ALK Kinase Assay

Assays quantitated ADP produced in reactions where ALK was incubated with ATP, polyGT substrate, buffer and varying concentrations of Tavocept or Crizotinib using the ADP-glo system by Promega. ALK phosphorylated the polyGT substrate using the ATP cofactor and produced ADP. Initially, 25 μL volumes were utilized for assays; subsequently half-area 96-well microplates were obtained that allowed the reduction of assay volume to 10 μL thereby significantly saving on reagents. The 25 μL volume assays in whole area 96-well microtiter plates contained ALK (100 ng total or 4 ng/μL), ATP (100 μM), PolyGT substrate (0.2 μg/μL) and, if applicable, varying concentrations of Tavocept and/or PF2341066 (Crizotinib); additionally, kinase assay buffer was added to achieve a final total assay volume of 254. The 10 μL volume assays in half area 96-well microtiter plates contained ALK (40 ng total or 4 ng/μL), ATP (100 μM), PolyGT substrate (0.2 μg/μL) and the concentrations of Tavocept and/or Crizotinib as indicated; additionally, kinase assay buffer was added to achieve a final volume of 10 μL per assay. For most assays a stock of ATP (1 mM) and PolyGT (2 mg/mL) was mixed 1:1 (v/v) to give an ATP/PolyGT master mix of 0.5 mM ATP and 1 mg/mL PolyGT. Crizotinib was dissolved as a 1 mM stock in DMSO and then further diluted in kinase assay buffer (DMSO only controls were always run to ensure that DMSO did not interfere with the assay). The reactions, in microtubes, were incubated for 60 minutes at 25° C. in a water bath. Following this 60 minute incubation, reactions were transferred to microplates and the kinase activity was evaluated using the ADP-glo system from Promega and monitored, in an endpoint assay, ADP produced when ALK phosphorylated the PolyGT substrate. Various controls were tested and included the assay components indicated in the columns of Table 9 below (where + indicates the component was included and an empty cell indicates it was not included). Any controls lacking ATP had extremely low to undetectable RLU signal (<600 RLU) and, therefore, not all of these controls are listed in Table 9.

TABLE 9
Description of Selected Controls

| | | | PolyGT | Solvent |
|---|---|---|---|---|
| Name of Control | ALK | ATP | Substrate | (DMSO) |
| DMSO only control (for Crizotinib titrations) | + | + | + | + |
| Background ATPase control | | + | | |
| Background Kinase Autophosphorylation Control | + | + | | |
| Background Kinase Interference Control, No Substrate | + | | | |
| Background Kinase Interference Control, With Substrate | + | | + | |
| Background Interference Control 1: PolyGT with Test | | | + | |
| Article Tavocept or Crizotinib | | | | |
| Background Interference Control 2: PolyGT and ATP | | + | + | |
| with Test Article Tavocept or Crizotinib | | | | |

### III. ADP-Glo Detection

Kinase assays were run in triplicate or quadruplicate in microplates. Following this, the ADP-glo detection system (Promega) was used to determine how much ADP had been produced. For 10 μL volume assays, to each microplate well containing 10 μL of kinase reaction was added ADP-glo reagent (10 μL), plates were spun in a table top centrifuge (1000 rpm (123×g) for 1 minute) to ensure no reagent remained on the well walls, and then agitated for 1 minute to ensure optimal mixing. Plates were incubated at 25° C. on a heat block for 40 minutes. Next, kinase detection reagent (20 μL) was added and, as above, centrifugation and agitation was repeated; plates were allowed to incubate at 25° C.±1° C. on a heat block for 40 minutes. Following the incubation of the kinase detection reagent, plates were read on a Tecan Ultra microplate reader. The Tecan Ultra contained a built-in plate definition file for the whole area 96-well white Corning plates but a plate definition file for the half area 96-well Corning plates was created using the RdrOle component of the Tecan Ultra software. For 25 μL volume assays, the procedure was as described above except that the ADP-glo reagent added to the 25 μL assay was 25 μL in volume and the kinase detection reagent was 50 μL in volume.

### VI. Evaluation of ALK Activity In Vitro and Determination of Assay Conditions

Kinases vary in their ability to turnover ATP in vitro; therefore, we evaluated the activity of the Millipore ALK over a concentration range from 0.4 ng/μL of assay to 4 ng/μL of assay (a range of 6.27 to 62.7 nM ALK). Turnover by ALK was relatively mediocre; therefore, the highest ALK concentration evaluated (4 ng/μL of assay) was utilized in the assays. This typically gave signal to background ratios for the control of 12 or higher (concentrations >4 ng/μL of assay gave even higher signal-to-background but were cost prohibitive).

Polyglutamate tyrosine (polyGT; 4:1 ratio) was used as the substrate for phosphorylation and had an average polymer mass ranging from 20,000 to 50,000 g/mole; each glu-glu-glu-glu-tyr “subpolymer” in this polymer has a mass of approximately 698 g/mole. Therefore, each mole of polymer of 20,000 g/mole would contain approximately 28 moles of the glu-glu-glu-glu-tyr “subpolymer.” Typically, a 10 μL assay would contain 2 μL of the polyGT substrate. Assuming the lower polymer mass of 20,000 g/mol mass, this translates to approximately 10 μM polyGT per assay and 280 μM glu-glu-glu-glu-tyr “subpolymer” per assay. This was a vast excess of possible tyrosine phosphorylation sites, ensuring that the substrate for phosphorylation was not rate limiting (assuming the higher mass range would produce an even larger excess of glu-glu-glu-glu-tyr “subpolymers”).

### V. Specific Experimental Results

Data from ALK assays run on the Tecan Ultra microplate spectrophotometer were collected in Microsoft Excel. Error calculations and graphical representations were performed in Microsoft Office Excel (Microsoft Corporation, Redmond, Wash., USA). Determination of IC₂₅, IC₅₀ and IC₇₅ values were accomplished using Origin Lab software (OriginLab Corporation, Northampton, Mass., USA).

A. Tavocept Inhibits ALK Activity In Vitro

Tavocept inhibited ALK with an IC₅₀ of 9.16±2.91 mM under assay conditions of 100 μM ATP and with an IC₅₀ of 20.80±3.49 mM under assay conditions of 500 μM ATP. These lower and higher ATP concentrations were used in an effort to see if Tavocept had either a competitive or non-competitive inhibitory effect, with respect to ATP binding, on ALK. Typically, in kinase endpoint assays like the Promega ADP-glo assay system, inhibitors are classified as competitive if their IC₅₀ increases notably as the ATP concentration increases. From previous structural work (ZTI-00-F6), it was observed that Tavocept covalently modified ALK on cys1156 in a loop region of ALK that may subsequently result in partial interference with the phosphate binding site for ALK's ATP cofactor. It also was observed in studies disclosed herein that as the ATP concentration was increased, the IC₅₀ for Tavocept also increased. Consequently, while the inhibition of ALK by Tavocept is not “classic” competitive inhibition (i.e., wherein ATP and Tavocept have nearly identical or at least significantly overlapping binding sites and only one molecule, either ATP or Tavocept, can occupy that site at a time), it is “competitive-like” based upon the increasing IC₅₀ as the ATP concentration is increased. Additionally, this classification is supported by the X-ray crystallography studies of the ALK structure containing a Tavocept adduct which indicate that Tavocept modification of ALK results in a perturbation of the P-loop near where the ATP binding site is located (FIG. 20).

Physiologically, concentrations of Tavocept as high as 18 mM have been achieved in the clinic. See, e.g., Verschraagen M, Boven E, Zegers I, Hausheer F H, van der Vijgh W J F. Pharmacokinetics of Tavocept and its metabolite mesna in plasma and ascites: a case report. Cancer Chemother. Pharmacol. 51(6):525-529 (2003). Tavocept has been administered at doses as high as 41 g/m² and Cₘₐₓ values in plasma of 10 mM are typical; therefore, the concentrations of Tavocept required to see an effect on ALK activity in vitro are physiologically relevant. ATP is often in the milliMolar range in vivo and the human body is reported to contain no more than 0.5 moles (˜250 g) of ATP at any time, but this supply is constantly and efficiently recycled. See, Id. In vivo there are many ATP-dependent enzymes that compete for ATP binding, including kinases, synthetases, helicases, membrane transporters and pumps, chaperones, motor proteins, and large protein complexes like the proteasome; therefore, the concentrations of 100 and 500 μM ATP used herein are approximations for ATP concentrations that may be available to ALK in vivo as it competes for ATP with the various other enzymes and proteins that utilize ATP.

B. Crizotinib Inhibits ALK Activity In Vitro

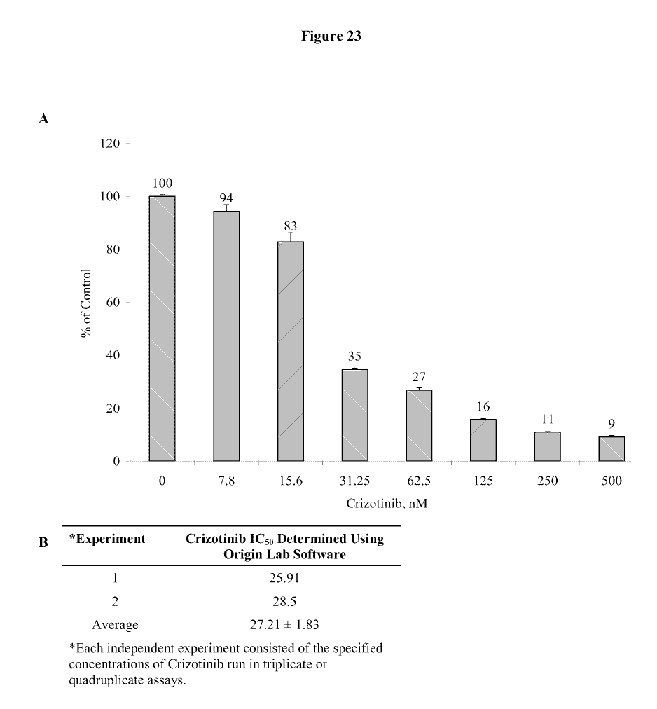

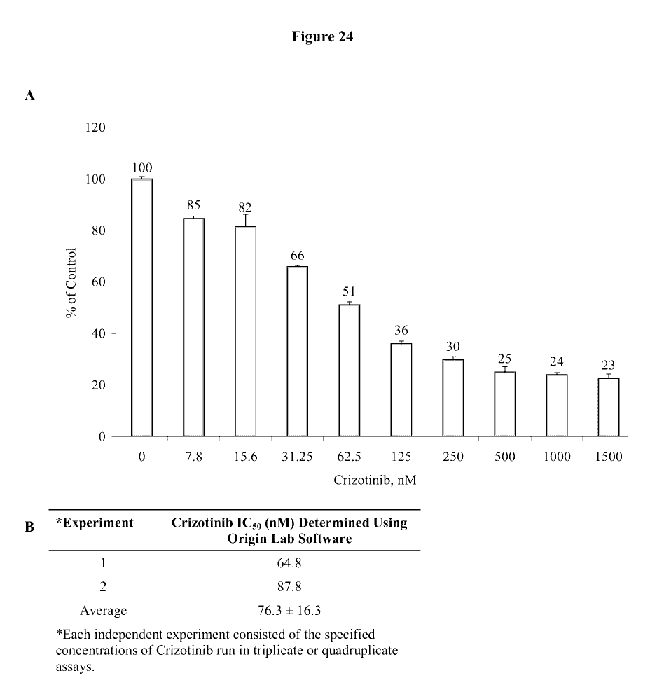

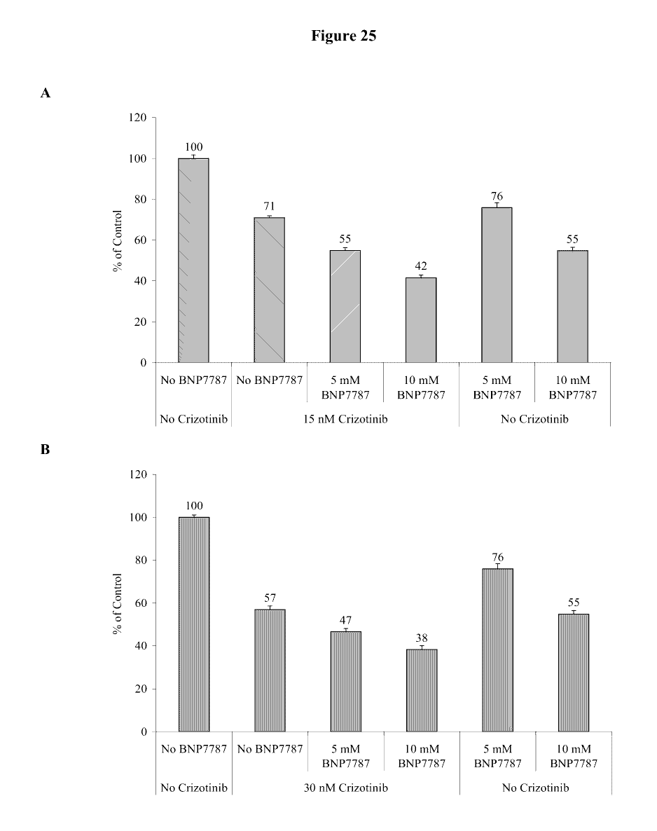

Crizotinib is a reported ATP-competitive inhibitor of ALK. In the in vitro kinase studies reported herein, the Applicants observed that Crizotinib inhibited ALK with an IC₅₀ of 27.2±1.83 (FIG. 23) under assay concentrations using ATP at 100 μM and with an IC₅₀ of 76.3±16.3 (FIG. 24) under assay concentrations using ATP at 500 μM. As mentioned above, Crizotinib has previously been characterized as a competitive inhibitor of ALK, with respect to ATP and the data disclosed herein is consistent with this previously reported observation. (Note that in clinical trials where Crizotinib was administered orally at doses of 250 mg twice daily, concentrations of Crizotinib of 57 nM were reported).

C. Tavocept Potentiates the Inhibitory Effect of Crizotinib on ALK Activity In Vitro Under 100 μM ATP Conditions

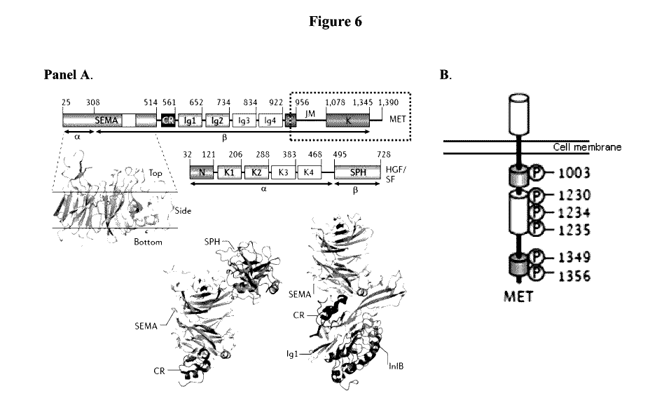

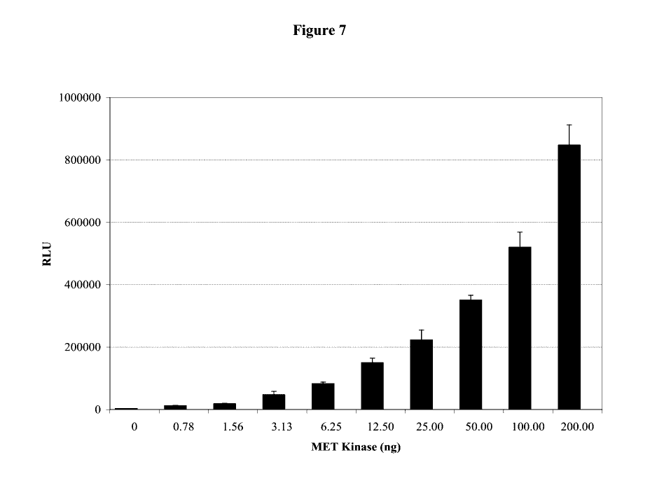

The effect of physiologically achievable concentrations of Tavocept near the IC₂₅ and IC₅₀ concentrations of Crizotinib were observed under assay conditions with either 100 μM (see, FIG. 6) or 500 μM (see, FIG. 24). Concentrations of Crizotinib of 57 nM been reported in clinical trials; therefore, concentrations used in these studies are within physiologically relevant ranges. Tavocept has been administered at doses as high as 41 g/m² and C., values in plasma of 10 mM are typical. See, e.g., Verschraagen M, Boven E, Zegers I, Hausheer F H, van der Vijgh W J F. Pharmacokinetics of Tavocept and its metabolite mesna in plasma and ascites: a case report. Cancer Chemother. Pharmacol. 51(6):525-529 (2003).

Tavocept notably potentiates the inhibitory effect of Crizotinib on ALK at physiologically relevant concentrations of both Tavocept and Crizotinib. In summary, FIG. 8 illustrates the effect of Tavocept on Crizotinib-mediated inhibition of ALK activity under 100 μM ATP conditions and 15 nM Crizotinib (Panel A) or 30 nM Crizotinib (Panel B). Under assay conditions with 100 μM ATP, 5 mM Tavocept in combination with 15 nM Crizotinib (near the IC₂₅ value for Crizotinib, when ATP is 100 μM) resulted in 16% greater inhibition than 15 nM Crizotinib alone whereas 10 mM Tavocept in combination with 15 nM Crizotinib resulted in 29% greater inhibition than 25 nM Crizotinib alone. Under assay conditions with 100 μM ATP, 5 mM Tavocept in combination with 30 nM Crizotinib (near the IC₅₀ value of Crizotinib) resulted in 10% greater inhibition than 30 nM Crizotinib alone whereas 10 mM Tavocept in combination with 30 nM Crizotinib resulted in 19% greater inhibition than 30 nM Crizotinib alone. These assays near the IC₅₀ value for Crizotinib (i.e., 30 nM, when ATP is 100 μM), have somewhat lower stimulation compared to 100 μM ATP and 15 nM Crizotinib conditions. For all of the ALK assays that contained 100 μM ATP, it was noted that ALK activity levels of less than 10% were not generally observed; this is a technical limitation of the assay, and this means that there is only a range of 40% between the IC₅₀ and the assay lower limit, so any additional stimulation by Tavocept is confined by this limit. As discussed in preceding sections, Tavocept alone or Crizotinib alone both were also effective at inhibiting ALK in vitro.

D. Tavocept Potentiates the Inhibitory Effect of Crizotinib on ALK Activity In Vitro Under 500 μL ATP Conditions

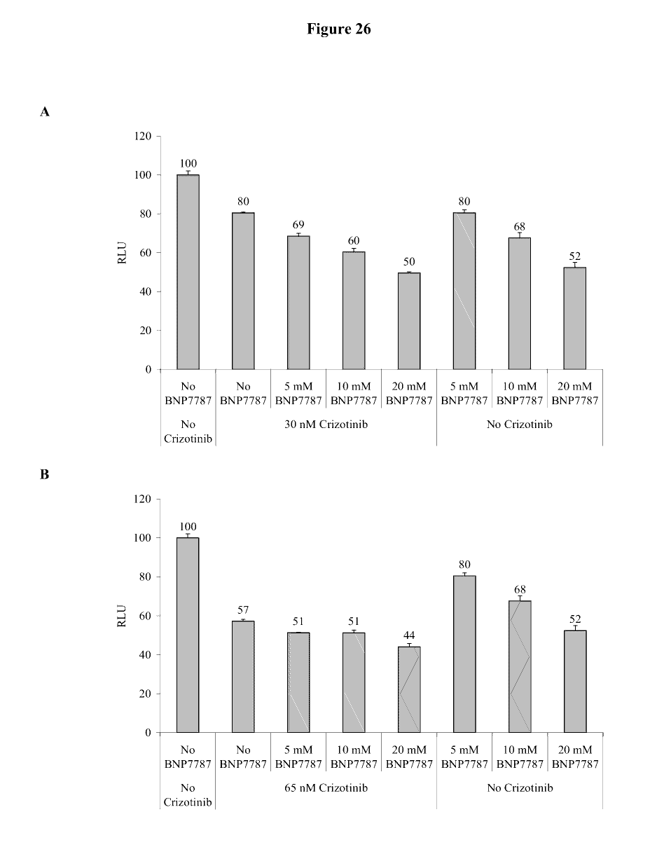

In summary, FIG. 26 illustrates the effect of Tavocept on Crizotinib-mediated inhibition of ALK activity under 500 μM ATP conditions at 30 nM Crizotinib (FIG. 26, Panel A) or 65 nM Crizotinib (Panel B). Under assay conditions with 500 μM ATP, 5 mM Tavocept in combination with 30 nM Crizotinib (near the IC₂₅ value for Crizotinib) resulted in 11% greater inhibition than 30 nM Crizotinib alone, 10 mM Tavocept in combination with 30 nM Crizotinib resulted in 20% greater inhibition than 30 nM Crizotinib alone, and 20 mM Tavocept in combination with 30 nM Crizotinib resulted in 30% greater inhibition than 30 nM Crizotinib alone. Under assay conditions with 500 μM ATP, both 5 and 10 mM Tavocept in combination with 65 nM Crizotinib (near the IC₅₀ value of Crizotinib) resulted in 6% greater inhibition than 65 nM Crizotinib alone whereas 20 mM Tavocept in combination with 65 nM Crizotinib resulted in 13% greater inhibition than 65 nM Crizotinib alone. These assays near the IC₅₀ values for Crizotinib (i.e., 65 nM) when ATP is 500 μM, have somewhat lower stimulation compared to 100 μM ATP conditions. For all of the ALK assays that contained 500 μM ATP, we noted that ALK activity levels of less than 25-30% were not generally observed; this is a technical limitation of the assay. And this means that there is only a range of 30-35% between the Crizotinib IC₅₀ and the assay lower limit, so any additional stimulation by Tavocept is confined by this limit. As discussed in preceding sections, Tavocept alone or Crizotinib alone were also effective at inhibiting ALK in vitro.

VI. Summary of Studies on ALK and Tavocept and/or Crizotinib Interactions

The results from this study support the following conclusions:
In assays with 100 μM ATP, Tavocept inhibits ALK with an IC₅₀ value of 9.16±2.91 mM.
In assays with 500 μM ATP, Tavocept inhibits ALK with an IC₅₀ value of 20.80±3.49 mM.
In assays with 100 μM ATP, Crizotinib inhibits ALK with an IC₅₀ value of 27.21±1.83 nM.
In assays with 500 μM ATP, conditions, Crizotinib inhibits ALK with an IC₅₀ value of 76.3±16.3 nM.
Tavocept and Crizotinib together, inhibit ALK more than either test article alone.
In assays with 100 μM ATP and 15 nM Crizotinib, 5 and 10 mM Tavocept, respectively, resulted in 16% and 29% greater inhibition than 15 nM Crizotinib alone.
In assays with 100 μM ATP and 30 nM Crizotinib, 5 and 10 mM Tavocept, respectively, resulted in 10% and 19% greater inhibition than 30 nM Crizotinib alone.
In assays with 500 μM ATP and 30 nM Crizotinib, 5, 10 and 20 mM Tavocept, respectively, resulted in 11%, 20% and 30% greater inhibition than 30 nM Crizotinib alone.
In assays with 500 μM ATP and 65 nM Crizotinib, 5, 10 and 20 mM Tavocept, respectively, resulted in 6%, 6% and 13% greater inhibition than 65 nM Crizotinib alone.
Tavocept modulates the activity of ALK in vitro, if this occurs in vivo, a potential survival benefit could accompany this ALK modulation in NSCLC patients bearing ALK fusions or ALK mutations.

(iii) ROS1

The c-ROS gene was first discovered in 1986 when a recombinant DNA clone containing cellular sequences homologous to the transforming sequence, v-ROS, of the avian sarcoma virus UR29-11 was isolated from a chicken genomic DNA library. UR2 sarcoma virus is a retrovirus of chicken that encodes for a fusion protein, P68^{gag-ROS}, having tyrosine-specific kinase activity. See, e.g., Feldman, R. A., Wang, L. H., et al. Avian sarcoma virus UR2 encodes a transforming protein which is associated with a unique protein kinase activity. J. Virol. 42:228-236 (1982). The oncogene, v-ROS, of UR2 carries a kinase domain that is homologous to those present in the oncogenes of the src family. The c-ROS sequence appeared to be conserved in vertebrate species, from fish to mammals (including humans). The comparison of the deduced amino acid sequence of c-ROS and that of v-ROS showed two differences: (i) v-ROS contains three amino acids insertion within the hydrophobic domain (TM domain), presumed to be involved in membrane association; and (ii) the twelve carboxy-terminal amino acids of v-ROS are completely different from those of the deduced c-ROS sequence. See, e.g., Neckameyer, W. S., Shibuya, M., Hsu, M. T., Wang, L. H. Proto-oncogene c-ROS codes for a molecule with structural features common to those of growth factor receptors and displays tissue-specific and developmentally regulated expression. Mol. Cell Biol. 6:1478-1486 (1986).

Early reports have indicated that the deduced amino acid sequence of the kinase domain of ROS is highly homologous to that of the kinase domain of the human insulin receptor (HIR). However, it was later determined that the amino acid sequences in the kinase domains of these two RTKs are highly different, as the homology level in the amino acid sequence in the kinase domains of ROS and HIR was found to be only 48.5%. See, e.g., Matsushime, H., Wang, L. H., Shibuya, M. Human c-ROS gene homologous to the v-ROS sequence of UR2 sarcoma virus encodes for a transmembrane receptor-like molecule. Mol. Cell Biol. 6:3000-3004 (1986). In addition, the overall structure of c-ROS gene showed that the encoded protein carries an extracellular domain with a potential site of N-linked glycosidation, a hydrophobic 24-amino acids stretch, and a tyrosine kinase domain. See, e.g., Id. These structural organizations are similar to those of: (i) c-ErbB (the gene of the epidermal growth factor receptor); (ii) c-Fms (the gene of macrophage colony-stimulating factor receptor); and (iii) the HIR gene. These results strongly suggested that the human ROS gene encodes for a transmembrane molecule which may function as a receptor for cell growth or differentiation factors. The analysis of c-ROS gene sequence applied to a transcript separated from rat lung and a cDNA from a human glioblastoma cell line (AW-1088) indicated a homology between the putative extracellular domain of ROS and the extracellular domain of the sevenless gene product of Drosophila melanogaster. Sevenless is a gene required for normal eye development in the fruit fly D. melanogaster and it also encodes a transmembrane tyrosine-specific protein kinase. See, e.g., Bowtell, D., Simon, M., Rubin, G. Nucleotide sequence and structure of the sevenless gene of Drosophila melanogaster. Genes Dev. 2:620-634 (1988). The c-ROS oncogene was proved to be a member of the src gene family (see, e.g., Bishop, J. M. Viral Oncogenes. Cell 42:23-28 (1985)) the proteins encoded by these genes have a high degree of amino acid sequence homology, and are all associated with tyrosine-specific kinase activities (see, e.g., Ullrich, A., Coussens, L., et al. Human epidermal growth factor receptor cDNA sequence and aberrant expression of the amplified gene in A431 epidermoid carcinoma cells. Nature 309:418-425 (1984)).

ROS1 is an orphan receptor (i.e., endogenous ligand unknown) that is highly expressed in many tumor cell lines and belongs to a subfamily of tyrosine kinase insulin receptor genes. ROS1 activates pathways critical for cell proliferation including, but not limited to, pathways that are linked to PI3K, Akt, STAT3, and VAV3. See, e.g., Acquaviva J, Wong R, Charest A. The multifaceted roles of the receptor tyrosine kinase ROS in development and cancer. Biochim. Biophys. Acta. 1795(1):37-52 (2009). ROS1 was identified as an oncogene more than two decades ago (see, Birchmeier, et al., Characterization of an activated human ROS gene. Mol. Cell Biol. 6(9):3109-3116 (1986)) and shortly thereafter rearrangements were identified in the most aggressive type of brain cancer, glioblastomas (see, e.g., Birchmeier, et al., Expression and rearrangement of the ROS1 gene in human glioblastoma cells. Proc. Natl. Acad. Sci. U.S.A. 84:9270-9274 (1987)). Many different ROS1 fusions have been reported including, but not limited to, ROS1 fusions with GOPC, CEP85L, CD74, CCDC6, or SLC34A2. See, e.g., Seo, et al., The transcriptional landscape and mutational profile of lung adenocarcinoma. Genome Res. 22(11):2109-2119 (2012).

In the last few years, there has been a renewed interest in ROS1 fusions and rearrangements because they have been detected in many more cancer types and are associated with resistance to apoptosis. See, Id. For example, ROS1 fusions and rearrangements are thought to occur in 2-4% of non-small cell lung cancer (NSCLC) patients which corresponds to up to 4000 new NSCLC cases each year in the United States. See, e.g., Roberts, Clinical use of crizotinib for the treatment of non-small cell lung cancer. Biologics: Targets. Ther. 7:91-101 (2013). ROS1 rearrangements or fusions in NSCLC adenocarcinoma patients may be particularly important in younger never-smokers and/or Asian patients. See, e.g., Bergethon K, Shaw A T, Ou S H, Katayama R, Lovly C M, McDonald N T, et al., ROS1 rearrangements define a unique molecular class of lung cancers, J. Clin. Oncol. 30(8):863-870 (2012). In China, it is estimated that by 2025, nearly 1 million people will be diagnosed with NSCLC each year. Since ROS1 mutations may be even more prevalent among people with Asian ethnicity, they are anticipated to account for substantial deaths in this growing NSCLC patient population. See, Id.

In addition to NSCLC, rearrangements and fusions of ROS1 have been reported to occur in a wide range of other cancers including, but not limited to, stomach cancer, colorectal cancer, ovarian cancer, breast cancer, and kidney cancer. See, e.g., David, et al., Molecular Pathways: ROS1 Fusion Proteins in Cancer. Med. Res. Review 31(5):794-818 (2013). ROS1 fusions also occur in a notable subset of bile duct cancers, a common hepatic cancer that accounts for 10-15% of a all liver-related cancers. See, e.g., Gu, et al., Survey of tyrosine kinase signaling reveals ROS kinase fusions in human cholangiocarcinoma. PLoS ONE 6(1):e15640 (2011). With the growing emphasis on personalized medicine, particularly in the field of NSCLC, agents that target ROS1 have the potential for strong clinical utility.

### c-ROS Gene Distribution and Function

The transmembrane RTK ROS shows a specific profile of expression, which is restricted primarily to distinct epithelial cells during embryonic development. See, e.g., Liu, Z. Z., Wada, J., et al. Comparative role of phosphotyrosine kinase domains of c-ROS and c-ret proto-oncogenes in metanephric development with respect to growth factors and matrix morphogens. Dev. Biol. 178:133-148 (1996). When c-ROS was first isolated from the chicken genome, tissues at various stages of development were analyzed, but only kidneys were found to contain a significant level of c-ROS DNA. Subsequently, the expression of c-ROS gene in rats was examined and cDNA fragments containing the entire coding sequence of the gene were molecularly cloned. See, e.g., Matsushime, H., Shibuya, M. Tissue-specific expression of rat c-ROS-1 gene and partial structural similarity of its predicted products with sev protein of Drosophila melanogaster. J. Virol. 164:2117-2125 (1990). The c-ROS gene was found to be expressed in a tissue-specific manner with c-ROS transcripts of varying sizes in different tissues, with transcripts isolated from lungs, kidneys, heart, and testis. The in vivo expression pattern of ROS in mice was also determined, where transient ROS expression was found during development, in kidneys, lungs, and intestine. See, e.g., Sonnenberg, E., Godecke, A., et al. Transient and locally restricted expression of the ROS proto-oncogene during mouse development. EMBO J. 10:3693-3702 (1991). It was also found that ROS mRNA is present in the caput segment of the epididymis of adult mice (see, e.g., Sonnenberg-Riethmacher, E., Walter, B., et al. The c-ROS tyrosie kinase receptor controls regionalization and differentiation of epithelial cells in the epididymis. Genes Dev. 10:1184-1193 (1996)), with the expression being found to be restricted to the epithelial cells of the epididymis.

In humans, ROS was found to be expressed throughout the human epididymis at varying levels, while absent from the proximal caput. See, e.g., Lé gare, C., Sullivan, R. Expression and localization of c-ROS oncogene along the human excurrent duct. Mol. Hum. Reprod. 10:697-703 (2004). Northern blot analysis of RNA, isolated from various adult human organs, has shown that the highest ROS expression was detected in the lungs. Size variants were also detected in RNA isolated from placenta and skeletal muscle tissues. See, e.g., Acquaviva, J., Wong, R., Charest, A. The multifaceted roles of the receptor tyrosine kinase ROS in development and cancer. Biochim. Biophys. Acta 1795:37-52 (2009). The expression pattern of ROS in different organs suggests that it may play a role in the mature functions of these organs beyond a purely developmental role. It is also important to note that generally cellular homologues to retroviral transforming genes play an important role in cellular growth and/or differentiation, and appear to have oncogenic potential that can be manifested after transduction by a retrovirus. The process of conversion from a normal proto-oncogene to a transforming oncogene involves either mutation and/or degradation.

### Oncogenic Expression of ROS

The human c-ROS gene was mapped to the human chromosome 6, region 6q16-6q22. This region of chromosome 6 is involved in nonrandom chromosomal rearrangement in specific neoplasias, including: acute lymphoplastic leukemia, malignant melanoma, and ovarian carcinomas. c-ROS gene over-expression and/or mutations were found mainly in brain and lung cancers, in addition to chemically-induced stomach cancer, breast fibroadenomas, liver cancer, colon cancer, and kidney cancer.

### ROS in Non-Small Cell Lung Cancer (NSCLC)

In a large-scale survey of tyrosine kinase activity in lung cancer, tyrosine kinase signaling was characterized in 41 NSCLC cell lines and over 150 NSCLC tumors. See, Rikova, K., Guo, A., et al. Global survey of phosphotyrosine signaling identifies oncogenic kinases in lung cancer. Cell 131:1190-1203 (2007). Profiles of phosphotyrosine signaling were generated and analyzed to identify known oncogenic kinases. Interestingly, ROS kinase was determined to be in the top-ten receptor tyrosine kinases (RTKs) found in both cell lines and tumors. RTKs in this survey were ranked according to phosphorylation rank (phosphorylation level/sample). The results revealed that ROS kinase was highly expressed in one tumor sample and in the NSCLC cell line (HCC78). See, Id. In addition to ROS over-expression in these samples, protein tyrosine phosphatase non-receptor type 11 (PTPN11) and Insulin receptor substrate-2 (IRS-2), earlier reported to be important downstream effectors of ROS in glioblastoma, were found to be highly phosphorylated in ROS-expressing samples. See, Rikova, K., Guo, A., et al. Global survey of phosphotyrosine signaling identifies oncogenic kinases in lung cancer. Cell 131:1190-1203 (2007). Furthermore, several microarray analyses of tumor specimens also revealed significantly elevated ROS-expression levels in 20-30% of patients with NSCLC. See, e.g., Bild, A. H., Yao, G., et al. Oncogenic pathway signatures in human cancers as a guide to targeted therapies. Nature 439:353-357 (2006). Contrasting the results found in brain tumors, elevated ROS expression in lung tumors was observed in both early- and late-stage tumors, suggesting a key role for ROS in the initiation or development rather than progression of lung tumors. See, e.g., Bonner, A. E., Lemon, W. J., et al. Molecular profiling of mouse lung tumors: association with tumor progression, lung development, and human lung adenocarcinomas. Oncogene 23:1166-1176 (2004).

### ROS in Brain Tumors

A number of RTKs are characteristic as markers for nervous system tumors. By way of example, the epidermal growth factor receptor (EGFR) and its associated oncogene Erb-B are noteworthy, as 45-50% malignant gliomas show evidence for EGFR amplification. See, e.g., Yamazaki, H., Fukui, Y., et al. Amplification of the structurally and functionally altered epidermal growth factor receptor gene (c-erbB) in human brain tumors. Mol. Cell Biol. 8:1816-1820 (1988). Other RTKs include: Neu (see, e.g., Bernstein, J. J., Anagnostopoulos, A. V., et al. Human-specific c-neu proto-oncogene protein overexpression in human malignant astrocytomas before and after xenografting. J. Neurosurg. 78:240-251 (1993)), platelet-derived growth factor (PDGF) receptor (see, e.g., Lokker, N. A., Sullivan, C. M., et al., Platelet-derived growth factor (PDGF) autocrine signaling regulates survival and mitogenic pathways in glioblastoma cells. Cancer Res. 62:3729-3735 (2002)), ROS (see, e.g., Jun, H. J., Woolfenden, S., et al. Epigenetic regulation of c-ROS receptor tyrosine kinase expression in malignant gliomas. Cancer Res. 69:2180-2184 (2009)).

In a survey of 45 different human cell lines, ROS was found to be expressed in 56% of glioblastoma-derived cell lines at high levels (i.e., ranging from 10 to 60 transcripts per cell), while not expressed at all or expressed minimally in the remaining cell lines. See, Birchmeier, C., Sharma, S., Wigler, M. Expression and rearrangement of the ROS gene in human glioblastoma cells. Proc. Natl. Acad. Sci. USA 84:9270-9274 (1987). Moreover, no expression of ROS gene was observed in normal, non-neoplastic brain tissues; thus, the high level of ROS expression in glioblastoma seems specific. In all the tested glioblastoma cell lines, the c-ROS encoded transcript was found to be 8.3 kb in size, except for the cell line U-118MG, where its size was found to be only 4.0 kb, which suggests that the glioblastoma cell line U-118MG produces a high level of an altered (truncated) ROS-encoded protein. The overexpression of ROS in surgical specimens was also shown by two subsequent independent analyses using RNase protection and cDNA hybridization techniques, where high levels of ROS expression in 33 and 40% of glioblastoma surgical tumors was reported. See, Mapstone, T., McMichael, M., Goldthwait, D. Expression of platelet-derived growth factors, transforming growth factors, and the ROS gene in a variety of primary human brain tumors. Neurosurgery 28:216-222 (1991); Watkins, D., Dion, F., et al. Analysis of onocogen expression in primary human gliomas: Evidence for increased expression of the ROS onocogene. Cancer Genet. Cytogenet. 72:130-136 (1994). The failure of ROS detection in lower grade astrocytomas, however, suggests that ROS may play a role in tumor progression rather than initiation. See, Mapstone, T., McMichael, M., Goldthwait, D. Expression of platelet-derived growth factors, transforming growth factors, and the ROS gene in a variety of primary human brain tumors. Neurosurgery 28:216-222 (1991).

### ROS in Stomach, Breast, Liver, Colon, and Kidney Cancers

c-ROS gene was found to be upregulated in gastric cancer induced by oral administration of N-methyl-NO-nitro-N-nitrosoguanidine (MNNG) in rat. See, Yamashita, S., Nomoto, T., et al. Persistence of gene expression changes in stomach mucosae induced by short-term N-methyl-NO-nitro-N-nitrosoguanidine treatment and their presence in stomach cancers. Mutat. Res. 549:185-193 (2004). ROS gene was one of six genes found to be persistently upregulated after 4 weeks from MNNG treatment. ROS gene was found also to be overexpressed (in a number of other genes) in fibroadenoma samples taken from breast tumors of five different patients. It was found to be expressed at levels more than two-fold higher than those in normal tissues. See, e.g., Eom, M., Han, A., et al. ROS expression in fibroadenomas of the breast. Pathol. Int. 58:226-232 (2008). In liver, the induction of hepatic progenitor cells activation in a rat model of liver injury was found to be associated with overexpression of ROS. In addition, overexpression of ROS was also observed in a rat hepatoma cell line. See, e.g., Yovchev, M. I., Grozdanov, P. N., et al. Novel hepatic progenitor cell surface markers in the adult rat liver. Hepatology 45:139-149 (2007). Recently, a global sequencing survey of all tyrosine kinases in 254 cell lines revealed three new ROS mutations in two colon adenocarcinoma and one kidney carcinoma cell lines. See, Ruhe, J. E., Streit, S., et al. Genetic alterations in the tyrosine kinase transcriptome of human cancer cell lines. Cancer Res. 67:11368-11376 (2007).

Studies in the specific example of ROS1 described herein were designed to evaluate the effect of Tavocept on ROS1 kinase activity in the presence and absence of the known ATP-competitive inhibitor, Crizotinib (PF-02341066). As discussed previously in the section on ALK, X-ray crystallography on human anaplastic lymphoma kinase (ALK), a kinase important in a subset of NSCLC patients, indicated that Tavocept (BNP7787) xenobiotically modifes human ALK on cysteine residues 1156 and 1235. The Tavocept-mediated xenobiotic modification of cysteine residues 1156 and 1235 by Tavocept (BNP7787) on ALK inhibited ALK and potentiated the inhibitory activity of Crizotinib. In additional to ALK-related non-small cell lung cancer (NSCLC), a subset of NSCLC patients have rearrangements/fusions of the ROS1 kinase gene. See, e.g., Bergethon K, Shaw A T, Ou S H, Katayama R, Lovly C M, McDonald N T, et al., ROS1 rearrangements define a unique molecular class of lung cancers, J. Clin. Oncol. 30(8):863-870 (2012); Rikova K, Guo A, Zeng Q, Possemato A, Yu J, Haack H, et al., Global survey of phosphotyrosine signaling identifies oncogenic kinases in lung cancer, Cell 131(6):1190-1203 (2007). Bergethon and co-workers have reported that most patients with ROS1 kinase-related alterations are non-smokers similar to the profile of patients who have ALK mutations/fusions. See, e.g., Bergethon K, Shaw A T, Ou S H, Katayama R, Lovly C M, McDonald N T, et al., ROS1 rearrangements define a unique molecular class of lung cancers, J. Clin. Oncol. 30(8):863-870 (2012).

A search for structural studies on ROS1 in the literature was performed, but none were reported. However, as ALK and ROS1 share a sequence identity of ˜50% (kinase domain), homology modeling was utilized by the Applicants of the present patent application to build a structure of ROS1 using an ALK structure (PDB 3L9P) as a template. The homology model of ROS1 kinase (see, FIG. 1) was structurally very similar to ALK (PDB 3L9P), and it was hypothesized that an interaction between Tavocept (BNP7787) and human ROS1 kinase might occur in a manner similar to that observed previously for Tavocept (BNP7787) and ALK. Similarly, it was also hypothesized that Crizotinib would inhibit ROS1 kinase. Accordingly, the studies disclosed herein were designed to determine if Tavocept (BNP7787) had any affect on ROS1 kinase activity in vitro and to determine if BNP7787 had any effect on Crizotinib-induced (PF-02341066) inhibition of ROS1 kinase activity in vitro.

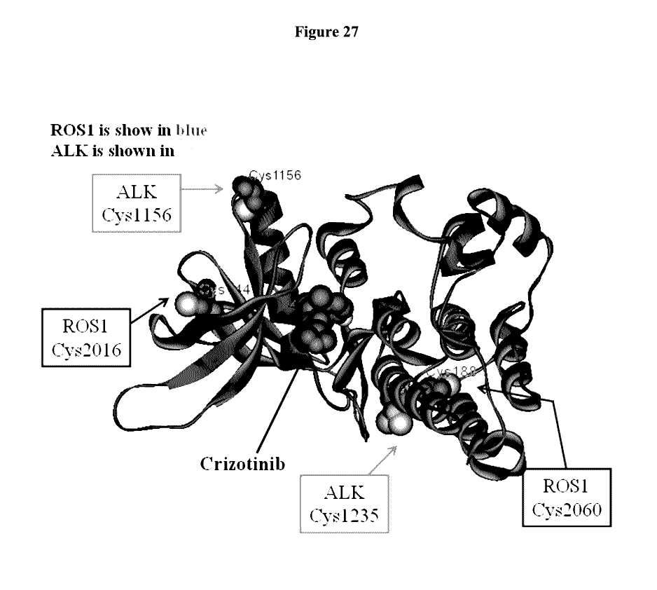

FIG. 27 illustrates a homology model of human ROS1 overlaid with the X-ray structure of human ALK (Protein Data Bank (PDB) entry for ALK was 3L9P). Backbone atom RMSD for ROS1 to ALK was 0.34 Å indicating very similar structures. ROS1 homology structure was prepared using Swiss PDB homology server and based on PDB entry 3L9P. See, e.g., Arnold K, Bordoli L, Kopp J, and Schwede T. The SWISS-MODEL Workspace: A web-based environment for protein structure homology modelling. Bioinformatics 22(2):195-201 (2006); Kiefer F, Arnold K, Künzli M, Bordoli L, Schwede T. The SWISS-MODEL Repository and associated resources. Nuc. Acids Res. 37:D387-D392 (2009). Sequence identity between ROS1 and ALK is approximately 50%. Previous studies indicated that Tavocept (BNP7787) xenobiotically modifies cys1156 of ALK. There is no cysteine in ROS1 kinase that corresponds to cys1156 in ALK and the nearest cysteine in ROS1 is cys2016. Also, in ALK, cys1235 is pointing to the outer surface in ALK and easily accessible. There is no cysteine in ROS1 kinase that corresponds to cys1235 in ALK and the nearest cysteine in ROS1 (cys2060) is pointing inside and is less accessible. This reduced accessibility could result in a slower or less efficient Tavocept (BNP7787)-mediated xenobiotic modification of ROS1 kinase.

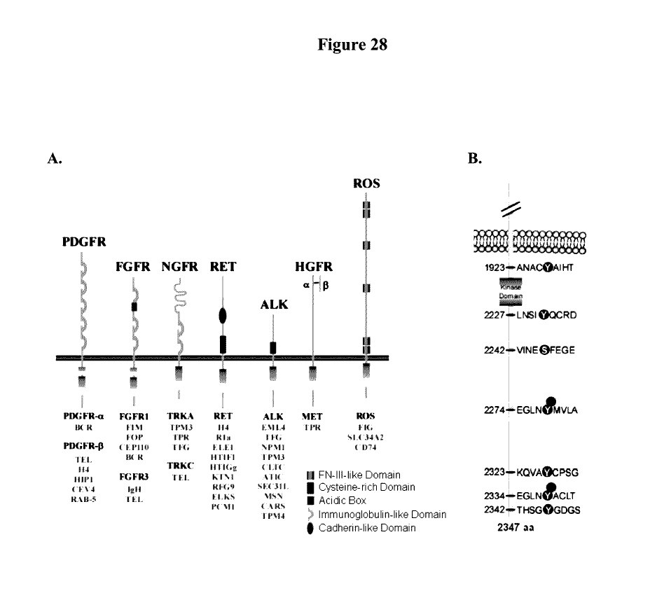

FIG. 28 illustrates the domain organization of ROS1. Panel A—Domain organization of ROS1 compared to other kinase receptors. Below each kinase, genes are listed that can fuse with the kinase (fused products may be involved in cancer or disease); Panel B—Intracellular kinase region of ROS1 including residues 1883-2347 with tyrosine (Y) and serine (S) phosphorylation sites identified.

### ROS1 Kinase Experimental Methodologies and Results

### I. Materials and Methods

Recombinant human ROS1 kinase (residues 1883-2347), containing an N-terminal GST tag and expressed in baculovirus Sf9, was purchased from SignalChem (lots R169-1, molecular weight=82 kDa) and aliquoted to 5-10 μL fractions of approximately 100 ng/μL when it was used the first time (so as to avoid multiple freeze/thaw cycles for subsequent experiments). BNP7787 was prepared by a proprietary method (lots #205001 or 450002-2, >97% pure, no mesna was detected by mass spectroscopy). Kinase inhibitor, PF-02341066 (also known as Crizotinib) was purchased from Selleck Chemicals, LLC (Cat. No. 877399-52-5, lot S1068802) and its structure is illustrated below. The substrate that was phosphorylated by the kinase, polyglutamate-tyrosine (PolyGT), was purchased from Sigma (P0275, lot 120M5007V).

### Structure of the ATP Competitive Inhibitor—Crizotinib

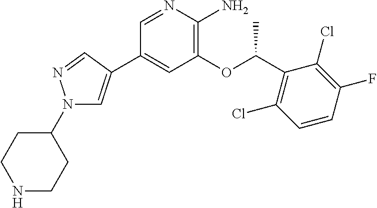 [SMILES: C[C@@H](Oc1cc(-c2cnn(C3CCNCC3)c2)cnc1N)c1c(Cl)ccc(F)c1Cl]

Kinase assay buffer was prepared and consisted of 20 mM HEPES (Sigma H-0891, lot 48H5432), 0.1% Brij 96 (aka Brij 35P, Sigma Aldrich 16005-2506-F, lot BCB07465V), 10 mM NaF (Sigma, S1504, lot 129H1425), 1 mM Na₃VO₄ (Sigma, S-6508, lot 061M0104), and 10 mM MnCl₂ (Sigma, M-5005, lot 108H0150) adjusted to a final pH of 7.5. Microplates were purchased directly from VWR and/or Corning and initial assay optimization was performed using whole area 96-well white microplates (Corning 3912, lot 29011050) but to save reagents and costs, later experiments were conducted in half area 96-well white microplates (Corning 3642, lot 05312045).

ADP-glo reagents were purchased from Promega and consisted of ADP (V916A, lot 32551702), ATP (V915A, lot 32559501), ADP-glo (V912A, lot 32559601 or V912B, lot 0000010953), kinase detection reagent buffer (V913A, lot 32179101 or V913B, lot 0000010953) and kinase detection substrate (V914A, lot 30286301 or V914B, lot 0000010722). All other reagents were purchased from Sigma Aldrich. A Tecan Ultra microplate reader with XFluor4 software (Tecan, V4.51) and RdrOle software (Tecan, V4.50) were used in this study.

### II. ROS1 Kinase Assay

Assays quantitated ADP produced in reactions where ROS1 incubated with ATP, polyGT substrate, buffer and varying concentrations of Tavocept (BNP7787) or Crizotinib using the ADP-glo system by Promega. ROS1 kinase phosphorylated the polyGT substrate using the ATP cofactor and produced ADP. Assays contained ROS1 kinase (5 ng total or 0.5 ng/μL), ATP (100 μM), PolyGT substrate (0.2 μg/μL) and the concentrations of Tavocept (BNP7787) and/or PF-02341066 (Crizotinib) as indicated; additionally, kinase assay buffer was added to achieve a final volume of 10 μL per assay. For most assays a stock of ATP (1 mM) and PolyGT (2 mg/mL) was mixed 1:1 (v/v) to give an ATP/PolyGT master mix of 0.5 mM ATP and 1 mg/mL PolyGT. Crizotinib was dissolved as a 5 mM stock in DMSO and then further diluted in kinase assay buffer (DMSO only controls were always run to ensure that DMSO did not interfere with the assay). The reactions, in microtubes, were incubated for 60 minutes at 25° C. in a water bath. Following this 60 minute incubation, 10 μL aliquots were transferred to microplates and the kinase activity was evaluated using the Promega ADP-glo system that monitored, in an endpoint assay, ADP produced when ROS1 kinase phosphorylated the PolyGT substrate.

It should be noted that numerous controls were tested and included the assay components indicated in the columns of Table 10 below (where + indicates the component was included and an empty space indicates it was not included). Controls that lacked ATP had extremely low to undetectable RLU signal (<600 RLU).

TABLE 10
Description of Selected Controls

| | ROS1 | | PolyGT | Solvent |
|---|---|---|---|---|
| Name of Control | kinase | ATP | Substrate | (DMSO) |
| DMSO only control (for Crizotinib titrations) | + | + | + | + |
| Background ATPase control | | + | | |
| Background Kinase Autophosphorylation Control | + | + | | |
| Background Kinase Interference Control, No | + | | | |
| Substrate | | | | |
| Background Kinase Interference Control, With | + | | + | |
| Substrate | | | | |
| Background Interference Control 1: PolyGT with | | | + | |
| Test Article BNP7787 or Crizotinib | | | | |
| Background Interference Control 2: PolyGT and | | + | + | |
| ATP with Test Article BNP7787 or Crizotinib | | | | |
| Background Interference Control 3: Substrate only | | | + | |

### III. ADP-Glo Detection

The Kinase Assays, as described in Section II above, were run in triplicate or quadruplicate in microplates. Following these assays, the ADP-glo detection system (Promega) was used to determine how much ADP had been produced.

For 10 μL volume assays, to each microplate well containing 10 μl of kinase reaction was added ADP-glo reagent (10 μL), the microplates were spun in a table top centrifuge (1000 rpm (123×g) for 1 minute) to ensure that no reagent remained on the walls of the individual wells, and then agitated for 1 minute to ensure optimal mixing. The microplates were incubated at 25° C. on a heat block for 40 minutes. Kinase detection reagent (20 μL) was then added and, as above, centrifugation and agitation was repeated; with the microplates being allowed to incubate at 25° C.±1° C. on a heat block for 40 minutes. Following the incubation of the kinase detection reagent, the microplates were read on a Tecan Ultra microplate reader. The Tecan Ultra contained a built-in plate definition file for the whole area 96-well, white Corning microplates; however a microplate definition file for the half area 96-well Corning plates was created using the RdrOle component of the Tecan Ultra software.

### IV. Evaluation of ROS1 Kinase Activity In Vitro and Determination of Assay

Conditions

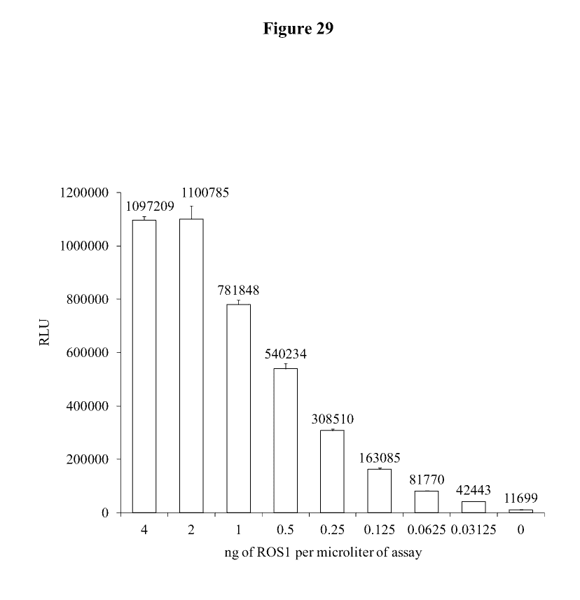

Kinases vary in their ability to turnover ATP in vitro; therefore, the activity of the SignalChem ROS1 kinase was evaluated over a concentration range from 0.031 to 4 ng/μL of assay (i.e., a range of 0.378 to 4.88 nM ROS1 kinase). Turnover by ROS1 was found to be robust and a concentration of 0.5 or 0.7 ng/μL was used in assays (see, FIG. 29). These concentrations typically gave signal to background ratios of approximately 15 or higher. Polyglutamate tyrosine (polyGT; 4:1 ratio) was used as the substrate for phosphorylation and had an average polymer mass ranging from 20,000 to 50,000 g/mole; wherein each glu-glu-glu-glu-tyr “subpolymer’ in this polymer has a mass of approximately 698 g/mole. Therefore, each mole of PolyGT polymer of 20,000 g/mole would contain approximately 28 moles of the glu-glu-glu-glu-tyr “subpolymer.”

Typically, a 10 μL assay volume would contain 2 μg of the polyGT substrate. Assuming the lower polymer mass of 20,000 g/mol mass, this translates to approximately 10 μM polyGT per assay and 280 μM glu-glu-glu-glu-tyr “subpolymer” per assay. This was a vast excess of possible tyrosine phosphorylation sites, thus ensuring that the substrate for phosphorylation was not rate limiting (assuming the higher mass range would produce an even larger excess of glu-glu-glu-glu-tyr “subpolymers”).

### V. Specific Experimental Results

Data from ROS1 kinase assays run on the Tecan Ultra microplate spectrophotometer were collected in Microsoft Excel. Error calculations and graphical representations were performed in Microsoft Office Excel (Microsoft Corporation, Redmond, Wash., USA). Determination of IC₂₅, IC₅₀ and IC₇₅ values were accomplished using Origin Lab software (OriginLab Corporation, Northampton, Mass., USA).

A. Crizotinib Inhibits ROS1 Kinase Activity In Vitro

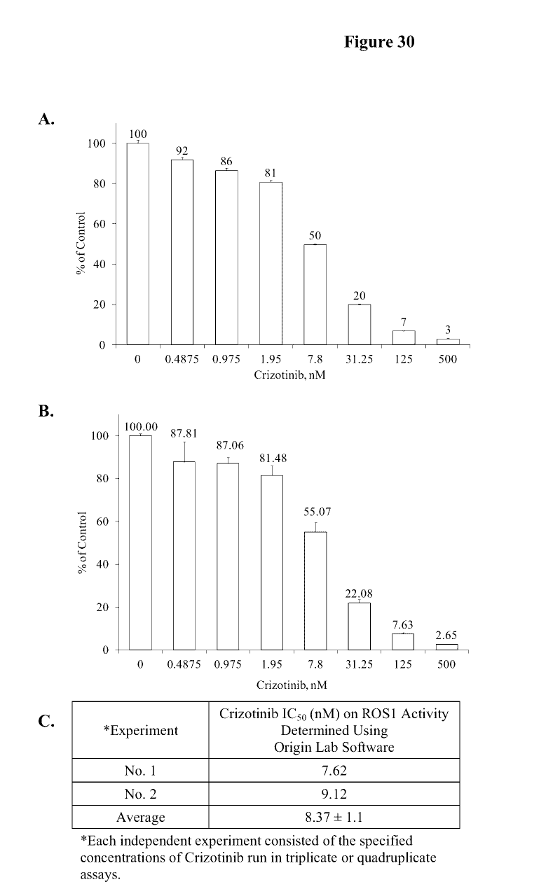

Crizotinib is a reported ATP-competitive inhibitor of ALK. See, e.g., Bang Y-J. The potential for crizotinib in non-small cell lung cancer: a perspective review. Ther. Adv. Med. Oncol. 3(6):279-291 (2011); Ou S-H. Crizotinib: a novel and first-in-class multitargeted tyrosine kinase inhibitor for the treatment of anaplastic lymphoma kinase rearranged non-small cell lung cancer and beyond. Drug Des. Devel. Ther. 5:471-485 (2011). In the in vitro kinase studies reported herein, we observed that crizotinib also potently inhibited ROS1 kinase with an IC₅₀ of 8.37 nM±1.1 nM (see, FIG. 30) under assay concentrations using ATP at 100 μM. As mentioned above, crizotinib has previously been characterized as a competitive inhibitor of ALK, with respect to ATP. See, Id. In order to understand how ATP interacts with the kinase domain of ROS1, the Applicants of the present patent application searched the literature and databases for structural studies on ROS1. Since there were no structural studies on ROS1 reported, and since ALK and ROS1 share a sequence identity of ˜50% (kinase domain). homology modeling was used to build a structure of ROS1 using ALK structure (3L9P) as template. See, FIG. 27. The aforementioned homology model of ROS1 kinase was very structurally similar to ALK, and it was hypothesized that crizotinib would inhibit ROS1 kinase. It was observed that crizotinib inhibited ROS1 with potency in the low nanomolar range. It should be noted that in clinical trials, where crizotinib was administered orally at doses of 250 mg twice daily, concentrations of crizotinib of 57 nM were reported. See, e.g., Ou S-H. Crizotinib: a novel and first-in-class multitargeted tyrosine kinase inhibitor for the treatment of anaplastic lymphoma kinase rearranged non-small cell lung cancer and beyond. Drug Des. Devel. Ther. 5:471-485 (2011).

B. Time-Dependent Effect of Tavocept on ROS1 Kinase Activity

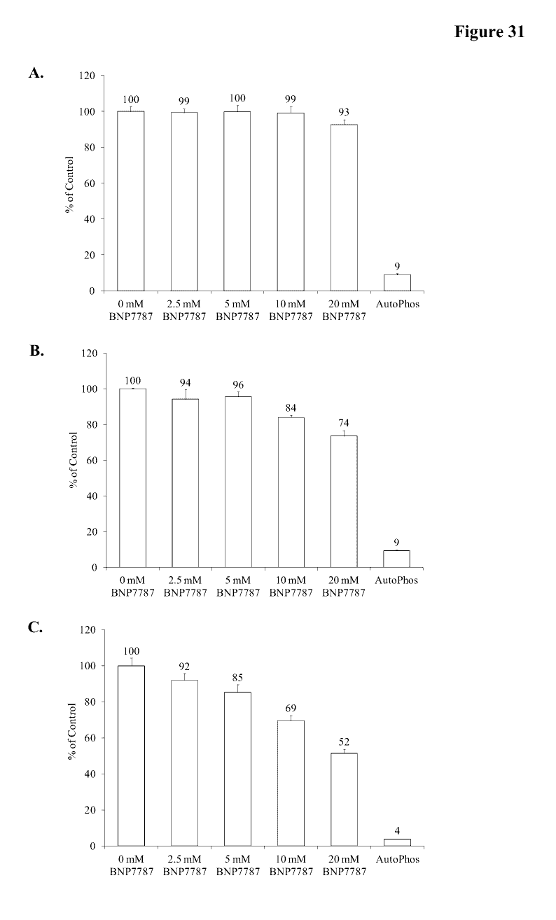

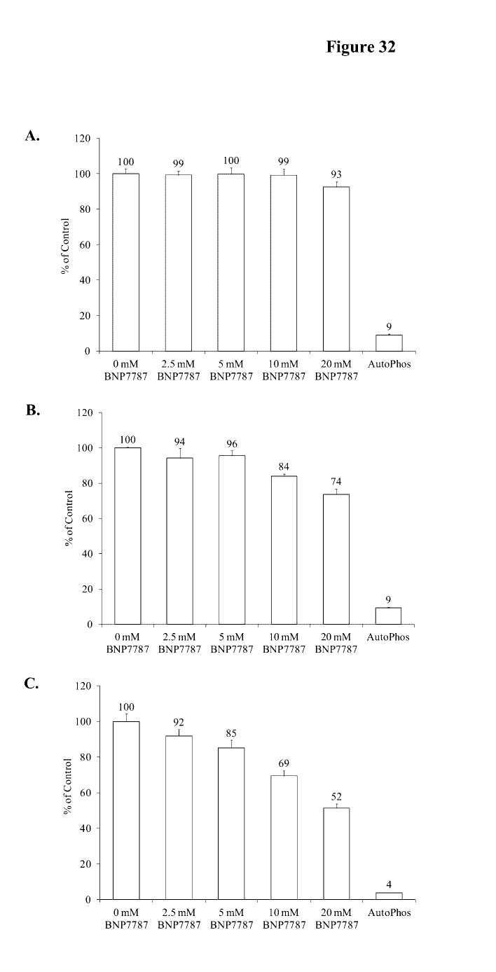

Tavocept (BNP7787) did not have a notable effect on ROS1 kinase activity in kinase assays where Tavocept (BNP7787) was added to ROS1 kinase simultaneously with ATP and polyGT substrate. See, FIG. 31. However, if ROS1 kinase was incubated with Tavocept (BNP7787) (3 hours or 24 hours), prior to addition of ATP and polyGT, a Tavocept (BNP7787)-mediated, time-dependent loss of activity was observed. See, FIG. 31, Panel B and Panel C. This time-dependent effect may be due to a slow or hindered reaction between a cysteine residue(s) on ROS1 kinase and Tavocept (BNP7787).

Physiologically, concentrations of Tavocept (BNP7787) as high as 18 mM have been achieved in the clinic and, currently, Tavocept (BNP7787) is administered at doses of 18.4 g/m² and Cₘₐₓ values in plasma of 10 mM and higher are typical. See, e.g., Verschraagen M, Boven E, Zegers I, Hausheer F H, van der Vijgh W J F. Pharmacokinetics of BNP7787 and its metabolite mesna in plasma and ascites: a case report. Cancer Chemother. Pharmacol. 51(6):525-529 (2003). Therefore, the concentrations of Tavocept (BNP7787) required to see an effect on ROS1 kinase activity in vitro are physiologically relevant. ATP is in the millimolar range in vivo, but in vivo there are many ATP requiring enzymes that compete for ATP binding; therefore, the concentration of 100 μM ATP used herein are thought to be good approximations for ATP concentrations that would be available to ROS1 kinase in vivo as it competes for ATP with the various other enzymes and proteins that utilize ATP.

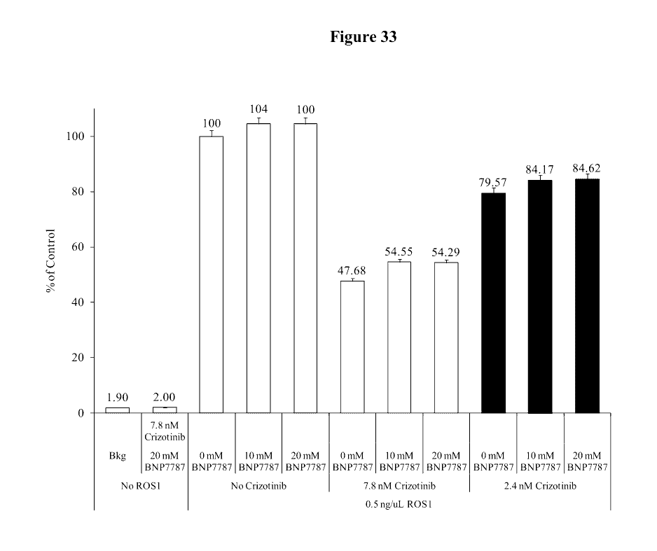

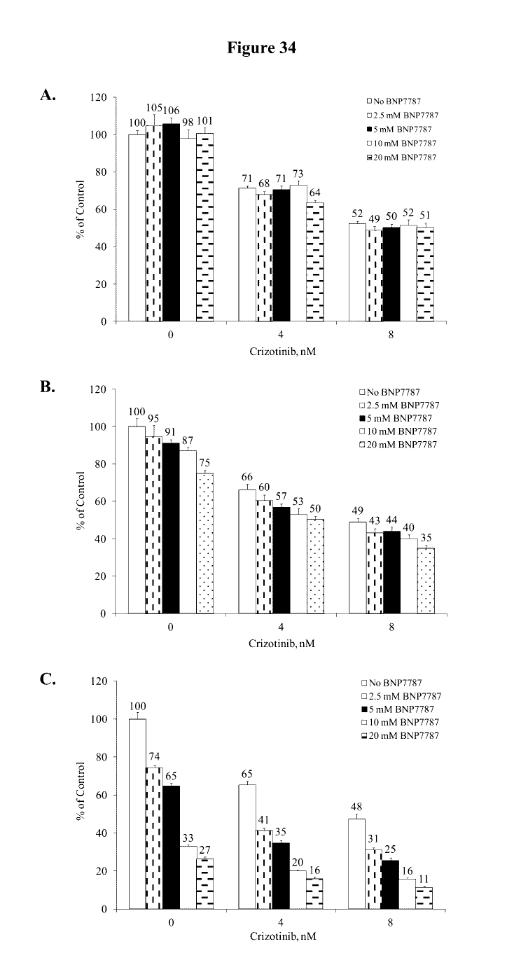

Tavocept (BNP7787) added simultaneously with crizotinib, ATP and polyGT did not affect crizotinib-mediated inhibition of ROS1 kinase activity in vitro. Under assay conditions with 100 μM ATP, 10 mM Tavocept (BNP7787) in combination with 2.4 or 7.8 nM crizotinib (near the IC₅₀ and IC₂₅ values, respectively, for crizotinib inhibition of ROS1 kinase) had no discernible effect on ROS1 kinase activity. See, FIG. 33. In these assays, the components needed for kinase activity (i.e., ATP and polyGT) were added simultaneously with Tavocept (BNP7787) and crizotinib.

Under assay conditions with 100 μM ATP, 10 mM Tavocept (BNP7787) in combination with 4 or 8 nM crizotinib had a time-dependent effect on ROS1 kinase activity. In these assays, the Tavocept (BNP7787) and ROS1 kinase were incubated together for 0, 3, or 24 hours prior to adding the assay components required for kinase activity (i.e., ATP and polyGT), and the kinase inhibitor, crizotinib.

As an example, refer to and compare the 4 nM crizotinib bars and note that all of the bars that represent reactions where Tavocept (BNP7787) was present (i.e., 2.5 to 20 mM) have lower percent of control values than the 4 nM crizotinib bar with no Tavocept (BNP7787) present (shaded solid gray). This general trend can be seen in all of reactions where Tavocept (BNP7787) is incubated with ROS1 kinase for 3 or 24 hours prior to addition of crizotinib, ATP, and polyGT. See, FIG. 34, Panel B and Panel C.

VI. Summary of Studies on ROS1 and Tavocept and/or Crizotinib Interactions

The results from this study support the following conclusions:
In assays with 100 μM ATP, crizotinib-inhibited ROS1 with an IC₅₀ value of 8.37±1.1 nM.
When Tavocept (BNP7787) (2.5-20 mM) was added simultaneously with ATP and polyGT, there was no discernible Tavocept (BNP7787)-mediated effect on ROS1 kinase activity.
When Tavocept (BNP7787) (2.5-20 mM) was added simultaneously with crizotinib, ATP and polyGT, there was no discernible Tavocept (BNP7787)-mediated stimulation of crizotinib inhibition of ROS1 kinase activity.
When 10 or 20 mM Tavocept (BNP7787) was incubated with ROS1 for 3 hours prior to addition of ATP and polyGT, losses of 16% and 26% of kinase activity, respectively, were observed.
When 5, 10, or 20 mM Tavocept (BNP7787) was incubated with ROS1 for 24 hours prior to addition of ATP and polyGT, losses of 15%, 31%, and 48% of activity, respectively, were observed.
When Tavocept (BNP7787) (2.5 to 20 mM) was incubated with ROS1 for 3 hours or 24 hours prior to addition of crizotinib, ATP and polyGT, Tavocept (BNP7787)-mediated stimulation of crizotinib inhibition of ROS1 kinase activity occurred in an additive manner.
The time dependent effects of Tavocept (BNP7787) on ROS1 kinase may indicate that Tavocept (BNP7787) would have a greater effect if administered prior to any agent that targets ROS1 kinase.
If Tavocept (BNP7787) modulates the activity of ROS1 in vivo, a potential survival benefit could accompany this modulation in, e.g., NSCLC and various other cancer patients bearing ROS1 kinase fusions or mutations.

(iv) Epidermal Growth Factor Receptor (EGFR)

There are approximately 20 classes of protein tyrosine kinases (PTKs), including the epidermal growth factor (EGF), insulin, PDGF, FGF, VEGF, and HGF receptor families. See, e.g., Hubbard, S. R., Miller, W. T. Receptor tyrosine kinases: mechanisms of activation and signaling. Curr. Opin. Cell Biol. 19:117-23 (2007). The EGF family (receptor tyrosine kinase class I) of membrane receptors, also called human epidermal receptor (HER) family, is one of the most relevant targets in this class. The epidermal growth factor receptor (EGFR) is the cell-surface receptor for members of the epidermal growth factor family (EGF-family) of extracellular protein ligands. See, e.g., Herbst, R. S. Review of epidermal growth factor receptor biology. Int. J. Radiat. Oncol. Biol. Phys. 59:21-26 (2004). EGFR is a member of the ErbB family of receptors, which comprise a subfamily of four (4) closely related receptor tyrosine kinases, which include: ErbB-1 (also known as epidermal growth factor receptor (EGFR), HER1); ErbB-2 (also know as HER 2 in humans and c-neu in rodents); ErbB-3 (also known as HER 3); and ErbB-4 (also known as HER 4). Mutations affecting EGFR expression and/or activity have been shown to be involved in many forms of cancer. EGFR (HER1, erbB1) is expressed or highly expressed in a variety of human tumors including, but not limited to: non-small cell lung cancer (NSCLC), breast, head and neck, gastric, colorectal, esophageal, prostate, bladder, renal, pancreatic, and ovarian cancers. See, e.g., Han, W., Lo, W-H. Landscape of EGFR Signaling Network in Human Cancers: Biology and Therapeutic Response in Relation to Receptor Subcellular Locations. Cancer Lett. 318:124-134 (2012). Table 11, below, illustrates the percent expressing EGFR in a number of solid tumor cancers. See, e.g., Laskin, J. J., Sandler, A. B. Epidermal growth factor receptor: a promising target in solid tumours. Cancer Treat. Rev. 30:1-17 (2004).

TABLE 11

| Tumor Type | % Expressing EGRF |
|---|---|
| Head & Neck | 80-100 |
| Colerectal | 25-77 |
| Pancreatic | 30-50 |
| Lung | 40-80 |
| Esophageal | 71-88 |
| Renal Cell | 50-90 |
| Prostate | 40-80 |
| Bladder | 53-72 |
| Cervical | 54-74 |
| Ovarian | 35-70 |
| Breast | 14-91 |
| Glioblastoma | 40-60 |

### ErbB Receptor Structure

ErbB receptors (170 kDa) are comprised of an extracellular region or ectodomain that contains approximately 620 amino acid residues, a single transmembrane-spanning region, and a cytoplasmic tyrosine kinase domain. The extracellular region of each ErbB family member is made up of four subdomains: L1, CR1, L2, and CR2—wherein “L” denotes a leucine-rich repeat domain and “CR” a cysteine-rich region. These subdomains are also referred to as domains I-IV, respectively. See, e.g., Ward, C. W., Lawrence, M. C., et al. The insulin and EGF receptor structures: new insights into ligand-induced receptor activation. Trends Biochem. Sci. 32:129-137 (2007). Viral ErbB receptor tyrosine kinases (v-ErbBs) have been shown to be homologous to EGFR, but lack sequences within the ligand binding ectodomain.

### ErbB Kinase Activation

The four members of the ErbB protein family are capable of forming homodimers, heterodimers, and possibly higher-order oligomers upon activation by a subset of potential growth factors ligands. Currently, a total of 11 growth factors have been identified that can activate ErbB receptors. The ability of each of these growth factors to activate the ErbB receptors is shown in Table 12, below; wherein the “+” and “−” symbols signify the ability and inability to activate each of the ErbB receptors, respectively. It should be noted that ErbB-2 has no known direct activating ligand, and may be in an activated state constitutively or become active upon heterodimerization with other family members (e.g., EGFR).

TABLE 12

ErbB Receptor

| Ligand | ErbB-1 | ErbB-2 | ErbB-3 | ErbB-4 |
|---|---|---|---|---|
| EGF | + | − | − | − |
| TGF-α | + | − | − | − |
| HB-EGF | + | − | − | + |
| amphiregulin | + | − | − | − |
| betacellulin | + | − | − | + |
| epigen | + | − | − | − |
| epiregulin | + | − | − | + |
| neuregulin 1 | − | − | + | + |
| neuregulin 2 | − | − | + | + |
| neuregulin 3 | − | − | − | + |
| neuregulin 4 | − | − | − | + |

When not bound to one of the aforementioned growth factor ligands, the extracellular regions of ErbB-1, -3, and -4 are found in a “tethered” conformation in which a 10 amino acid residue-long dimerization arm is unable to mediate monomer-monomer interactions. In contrast, in growth factor ligand-bound ErbB-1 and non-ligand-bound ErbB-2, the dimerisation arm becomes untethered and exposed at the receptor surface, thus making monomer-monomer interactions and dimerization possible. See, e.g., Linggi, B., Carpenter, G. ErbB receptors: new insights on mechanisms and biology. Trends Cell Biol. 16: 649-656 (2006). The consequence of ectodomain dimerization is the positioning of two cytoplasmic domains such that transphosphorylation of specific tyrosine, serine, and thronine amino acids can occur within the cytoplasmic domain of each ErbB species. Currently, at least ten specific tyrosine, seven serine, and two threonine amino acid residues have been identified within the cytoplamic domain of ErbB-1, that may become phosphorylated (and in some cases de-phosphorylated (e.g., Tyr⁹⁹²)) upon receptor dimerization. Although a number of potential phosphorylation sites exist, upon dimerization only one (or much more rarely two) of these sites are phosphorylated at any one time. See, e.g., Wu, S. L., Kim, J., et al. Dynamic profiling of the post-translational modifications and interaction partners of epidermal growth factor receptor signaling after stimulation by epidermal growth factor using Extended Range Proteomic Analysis (ERPA). Mol. Cell Proteomics. 5:1610-1627 (2006).

### EGFR Function

EGFR exists on the cell surface and is activated by binding of its specific ligands, including epidermal growth factor and transforming growth factor α (TGFα). As previously discussed, ErbB2 has no known direct activating ligand, and may be in an activated state constitutively or become active upon heterodimerization with other ErbB family members. Upon activation by its growth factor ligands, EGFR undergoes a transition from an inactive monomeric form to an active homodimer. However, there is also some evidence that preformed inactive dimers may also exist before growth factor ligand binding. In addition to forming homodimers, EGFR may pair with another member of the ErbB receptor family (e.g., ErbB2/Her2/neu) to create an activated heterodimer. There is also evidence to suggest that clusters of activated EGFRs form, although it remains unclear whether this clustering is important for activation itself or occurs subsequent to activation of individual dimers.

EGFR dimerization stimulates its intrinsic intracellular protein/tyrosine kinase activity. As a result, autophosphorylation of several tyrosine amino acid residues in the carboxy-terminal domain of EGFR occurs. These include Tryr⁹⁹², Tyr¹⁰⁴⁵, Tyr¹⁰⁶⁸, Tyr¹¹⁴⁸, and Tyr¹¹⁷³. See, e.g., Downward, J., Parker, P., Waterfield, M. D. Autophosphorylation sites on the epidermal growth factor receptor. Nature 311:483-485 (1984). This autophosphorylation elicits downstream activation and signaling by several other proteins that associate with the phosphorylated tyrosines through their own phosphotyrosine-binding SH2 domains. These downstream signaling proteins initiate several signal transduction cascades (principally the MAPK, Akt, and JNK pathways), leading to DNA synthesis and cell proliferation. See, e.g., Oda, K., Matsuoka, Y., et al. A comprehensive pathway map of epidermal growth factor receptor signaling. Mol. Syst. Biol. 1:205-210 (2005). Such proteins modulate phenotypes, including but not limited to: cell migration, cell adehsion, and cell proliferation. In addition, activation of the receptor is important for the innate immune response in human skin. See, e.g., Roupë, K. M.; Nybo, M., et al. Injury is a major inducer of epidermal innate immune responses during wound healing. J. Investigative Dermatol. 130:1167-1177 (2010). The kinase domain of EGFR can also cross-phosphorylate tyrosine residues of other receptors it is aggregated with and can itself, be activated in that same manner. See, e.g., Oda, K., Matsuoka, Y., et al. A comprehensive pathway map of epidermal growth factor receptor signaling. Mol. Syst. Biol. 1:205-210 (2005).

### EGFR Signaling

The importance of EGF-EGFR in protein phosphorylation and in tumorigenesis, and subsequently the EGF-EGFR signaling axis has taken an important role in developmental biology and cancer research. Activated EGFR recruits a number of downstream signaling molecules, leading to the activation of several major pathways that are important for tumor growth, progression, and survival. See, e.g., Lo, H. W., Hung, M. C. Nuclear EGFR signalling network in cancers linking EGFR pathway to cell cycle progression, nitric oxide pathway and patient survival. Br. J. Cancer 94:184-188 (2006). The main pathways downstream of EGFR activation include those mediated by PLC-γ-PKC, Ras-Raf-MEK, PI-3K-Akt-mTOR, and JAK2-STAT3. Similar to EGFR, the EGFRvIII variant is primarily localized on the cell-surface where it activates several signaling modules. However, unlike EGFR, EGFRvIII is constitutively active independent of ligand stimulation, in part, due to its loss of a portion of the ligand-binding domain.

While EGFR over-expression is found in many types of human cancers, EGFRvIII is predominantly detected in malignant gliomas. Both EGFR and EGFRvIII play critical roles in tumorigenesis and in supporting the malignant phenotypes in human cancers. Consequently, both receptors are targets of anti-cancer therapy. Several EGFR-targeting small molecule kinase inhibitors and therapeutic antibodies have been approved by the FDA to treat patients with breast cancer, colorectal cancer, non-small cell lung cancer (NSCLC), squamous cell carcinoma of the head and neck, and pancreatic cancer. Despite the extensive efforts invested in the preclinical and clinical development of EGFR-targeted therapy, the currently utilized treatments have demonstrated only modest effects on most cancer types, with the exception of NSCLC that expresses gain-of-function EGFR mutants. However, almost all of these aforementioned NSCLC patients eventually developed resistance to small molecule EGFR kinase inhibitors. See, e.g., Bonanno, L., Jirillo, A., Favaretto, A. Mechanisms of acquired resistance to epidermal growth factor receptor tyrosine kinase inhibitors and new therapeutic perspectives in non small cell lung cancer. Curr. Drug Targets 12:922-933 (2011). This acquired resistance has been shown to be linked to a secondary EGFR T790M mutation in approximately half of patients. This resistance can be attributed to other potential mechanisms, such as, uncontrolled activation of MET (see, e.g., Engelman, J. A., Janne, P. A. Mechanisms of acquired resistance to epidermal growth factor receptor tyrosine kinase inhibitors in non-small cell lung cancer. Clin. Cancer Res. 14:2895-2899 (2008)) and subsequent MET-mediated HER3 activity (see, e.g., Arteaga, C. L. HER3 and mutant EGFR meet MET. Nat. Med. 13:675-677 (2007)) and activated insulin-like growth factor-1 receptor (see, e.g., Morgillo, F., Kim, W. Y., et al. Implication of the insulin-like growth factor-IR pathway in the resistance of non-small cell lung cancer cells to treatment with gefitinib. Clin. Cancer Res. 13:2795-2803 (2007)). As lung cancer-associated EGFR mutations are either absent or very rare in other tumor types, there is an important need to identify the mechanisms underlying tumor resistance to anti-EGFR agents in order to derive sensitization strategies that can be used to overcome this resistance.

With respect to the need for gaining a deeper understanding of the EGFR pathway and EGFR-associated malignant biology in human cancer, compelling evidence indicates that plasma membrane-bound EGFR can mediate cellular processes independent of its kinase activity. This atypical mode of EGFR signaling could potentially contribute to the failure of the majority of EGFR-targeted agents that are designed to inhibit its kinase activity. Also compelling are the facts that both EGFR and EGFRvIII undergo nuclear and mitochondrial transport and that, within these organelles, the receptors exert novel functions that are distinctly different from their classical role as a receptor tyrosine kinase. See, e.g., Hung, M. C., Link, W. Protein localization in disease and therapy. J. Cell Sci. 124:3381-3392 (2011). To date, EGFR nuclear accumulation has been linked to several malignant phenotypes of human cancers, including: (i) proliferation; (ii) inflammatory response; (iii) DNA repair and therapeutic resistance; and (iv) poor clinical outcomes in cancer patients. See, e.g., Wheeler, D. L., Dunn, E. F., Harari, P. M. Understanding resistance to EGFR inhibitors-impact on future treatment strategies. Nat. Rev. Clin. Oncol. 7:493-507 (2010). While it has become clear that both EGFR and EGFRvIII undergo ligand- and treatment-induced mitochondrial localization, the regulation and consequences of the mitochondrial mode of EGFR signaling are still poorly understood despite being actively investigated.

### Cell-Surface and Cytoplasmic Modes of EGFR Signaling

### Kinase-Dependent Functions

The best known ligands of EGFR include: EGF, transforming growth factor-α, and heparin-binding EGF-like growth factor. Upon ligand binding, activated EGFR recruits, phosphorylates, and activates a number of important signaling molecules such as PLC-γ, Ras, PI-3K, and JAK2. Activated EGFR also phosphorylates signal transducer and activator of transcription-3 (STAT3) at Y705 and activates its dimerization, nuclear transport, and subsequent gene regulation. By way of example, EGFR-activated STAT3 has been shown to activate the expression of an E-cadherin repressor, TWIST, and thereby, promote epithelial-mesenchymal transition. These EGFR downstream signaling cascades can also be activated via EGFR-independent mechanisms; thereby regulating tumorigenesis, tumor proliferation and progression, and therapeutic resistance. See, e.g., Craven, R. J., Lightfoot, H., Cance, W. G. A decade of tyrosine kinases: from gene discovery to therapeutics. Surg. Oncol. 12:39-49 (2003).

### Kinase-Independent Functions

Independent of kinase activity or ligand activation, EGFR has been shown to mediate cellular processes mostly through its ability to physically interact with other proteins. One of the first observations suggesting this interesting phenomenon derived from the notion that loss of EGFR kinase activity did not lead to the phenotypes similar to ablation of EGFR expression. In this context, EGFR knockout animals were found to survive for up to eight days after birth and suffer from impaired epithelial development in several organs including skin, lung and gastrointestinal tract; whereas the animals with kinase-dead EGFR were viable despite having skin and eye abnormalities. In line with these findings, it was subsequently shown that the kinase-dead EGFR D813A mutant retained the ability to stimulate DNA synthesis. See, e.g., Coker, K. J., Staros, J. V., Guyer, C. A. A kinase-negative epidermal growth factor receptor that retains the capacity to stimulate DNA synthesis. Proc. Natl. Acad. Sci. USA 91:6967-6971 (1994). Co-expression of the kinase-dead EGFR K721M mutant with HER2 rescued the inability of the mutant EGFR to activate Akt and MAPK, suggesting that heterodimerization with other members of the ErbB family of receptors may help support the kinase-independent function of EGFR. See, e.g., Deb, T. B., Su, L., et al. Epidermal growth factor (EGF) receptor kinase-independent signaling by EGF. J. Biol. Chem. 276:15554-15560 (2001). In agreement with these reports, Ewald, et al. (Ligand- and kinase activity-independent cell survival mediated by the epidermal growth factor receptor expressed in 32D cells. Exp. Cell Res. 282:121-131 (2003)) showed that the kinase-dead EGFR K721R mutant retained the ability to survive serum starvation-induced death, while losing its ability to respond to EGF or to stimulate cell growth. Interestingly, the same study found another kinase-dead EGFR mutant D813A to lose both growth-stimulating and pro-survival properties, suggesting that the prosurvival activity of EGFR is independent of the kinase activity, but likely dependent of its unique structural properties to associate with other cellular proteins. This is also in-line with a more recent report showing that loss of expression of EGFR, but not its kinase activity, resulted in autophagic cell death. See, e.g., Weihua, Z., Tsan, R., et al. Survival of cancer cells is maintained by EGFR independent of its kinase activity. Cancer Cell 13:385-393 (2008). Specifically, these authors found that reduced intracellular glucose levels, leading to autophagy in EGFR-deficient cells, was due to the degradation of sodium/glucose cotransporter 1, SGLT1, a plasma membrane-bound protein that enables glucose uptake. Interestingly, cell-surface EGFR was found to physically interact with and stabilize SGLT1 independent of its kinase activity, thereby maintaining high glucose levels in the cells. Conversely, EGFR expression knockdown, but not kinase inhibition, led to SGLT1 degradation, reduction in intracellular glucose and subsequent autophagic cell death. Id. In support of these observations, co-expression of EGFR and SGLT1 was also found in both cell lines and specimens of oral squamous cell carcinoma. See, e.g., Hanabata, Y., Nakajima, Y., et al. Co-expression of SGLT1 and EGFR is associated with tumor differentiation in oral squamous cell carcinoma. Odontology (2011).

It is through physical associations, rather than kinase activity, that EGFR modulates protein subcellular trafficking. It has recently been reported that both EGFR and EGFRvIII associate with p53-upregulated modulator of apoptosis (PUMA), a proapoptotic member of the Bcl-2 family of proteins primarily located on the mitochondria. See, e.g., Zhu, H., Cao, X., et al. EGFR and EGFRvIII interact with PUMA to inhibit mitochondrial translocalization of PUMA and PUMA-mediated apoptosis independent of EGFR kinase activity. Cancer Lett. 294:101-110 (2010). PUMA is a potent apoptosis inducer that binds to and inhibits all five anti-apoptotic proteins (see, e.g., Chipuk, J. E., Fisher, J. C., et al. Mechanism of apoptosis induction by inhibition of the anti-apoptotic BCL-2 proteins. Proc. Natl. Acad. Sci. USA. 105:20327-20332 (2008)); whereas most BH3-only proteins only selectively engage anti-apoptotic proteins. PUMA also directly binds to the apoptotic executor BAX to induce mitochondrial outer membrane permeabilization. See, e.g., Gallenne, T., Gautier, F., et al. Bax activation by the BH3-only protein PUMA promotes cell dependence on antiapoptotic Bcl-2 family members. J. Cell Biol. 185:279-290 (2009). PUMA also strongly induces apoptosis in colorectal cancer, malignant gliomas, and in adult stem cells. It was further demonstrated that the EGFR-PUMA and EGFRvIII-PUMA interactions are independent of EGF stimulation or kinase activity and that these interactions are constitutive and only modestly reduced following apoptotic stress. See, e.g., Zhu, H., Cao, X., et al. EGFR and EGFRvIII interact with PUMA to inhibit mitochondrial translocalization of PUMA and PUMA-mediated apoptosis independent of EGFR kinase activity. Cancer Lett. 294:101-110 (2010). As a consequence of the EGFR-PUMA and EGFRvIII-PUMA interactions, PUMA is sequestered in the cytoplasm and unable to translocate onto the mitochondria to initiate apoptosis. This interesting observation is in agreement with the evidence showing that PUMA is highly co-expressed with EGFR/EGFRvIII in cell lines and primary specimens of malignant gliomas and that this particular tumor type has been found to be highly resistant to apoptosis-inducing treatments. Id.

### Nuclear Mode of EGFR Signaling

### Detection of Nuclear EGFR and EGFRvIII

Nuclear existence of EGFR was first observed in hepatocytes that underwent regeneration more than two decades ago. EGFR ligands, EGF, and pro-TGF-α, were also found to translocate into the nucleus of proliferating hepatocytes. Nuclear expression of EGFR was further detected in other types of normal cells and tissues, such as placenta, thyroid, immortalized epithelial cells of ovary and kidney origins, and keratinocytes. More recently, nuclear EGFR has been shown to be detected in many different types of cancer cells and specimens, including those of breast, epidermoid, bladder, ovary, oral cavity, lungs, pancreas, and in malignant gliomas. Nuclear EGFR can be localized within the nucleoplasm (see, e.g., Lin, S. Y., Makino, K., et al. Nuclear localization of EGF receptor and its potential new role as a transcription factor. Nat. Cell Biol. 3:802-808 (2001)) and on the inner nuclear membrane (see, e.g., Kim, J., Jahng, W. J., et al. The phosphoinositide kinase PIK mediates Epidermal Growth Factor Receptor trafficking to the nucleus. Cancer Res. 67:9229-9237 (2007)). Evidence to date indicates nuclear EGFR to be the full-length receptor that originates from the cell-surface. Analysis for nuclear presence of EGFRvIII has not been extensively conducted; however the presently available information has shown that EGFRvIII can be detected in prostate cancer and in malignant gliomas.

### Nuclear EGFR and EGFRvIII as Transcriptional Regulators

The role of EGFR in regulating gene regulation independent of its kinase activity was established in a milestone study which defined nuclear EGFR as a transcriptional co-factor that contains a transactivation domain in its C-terminus. See, Lin, S. Y., Makino, K., et al. Nuclear localization of EGF receptor and its potential new role as a transcription factor. Nat. Cell Biol. 3:802-808 (2001). This study also showed that nuclear EGFR associated with a consensus A/T-rich sequence within the human cyclin D1 promoter and that (following binding) cyclin D1 gene expression was upregulated. The transcriptional targets of nuclear EGFR that have been identified to date include: cyclin D1, inducible nitric oxide synthase (iNOS), B-Myb, cyclooxygenase-2 (COX-2), aurora A, c-Myc, and breast cancer resistance protein (BCRP). See, e.g., Han, W., Lo, W-H. Landscape of EGFR signaling network in human cancers: Biology and therapeutic response in relation to receptor subcellular locations. Cancer Lett. 318:124-134 (2012). Through increasing the expression of these target genes, nuclear EGFR has been linked to several malignant phenotypes of human cancers, including proliferation, inflammation and tumor drug resistance. See, e.g., Wang, Y. N., Yamaguchi, H., et al. Nuclear trafficking of the epidermal growth factor receptor family membrane proteins. Oncogene 29:3997-4006 (2010).

Given the fact that EGFR lacks a DNA-binding domain, extensive efforts have been focused on finding its transcriptional co-regulators with DNA-binding capability. These efforts have opened up new avenues of research. For example, Lo, et al., (Nuclear interaction of EGFR and STAT3 in the activation of iNOS/NO pathway. Cancer Cell 7:575-589 (2005)) reported that nuclear EGFR is able to associate with STAT3 oncogenic transcription factor to enhance expression of inducible nitric oxide synthase (iNOS), a protein involved in inflammation, tumor progression and metastasis. The same group further reported that nuclear EGFR interacted with E2F1 to activate human B-Myb gene expression, leading to uncontrolled proliferation. See, Hanada, N., Lo, H-W., et al. Co-regulation of B-Myb expression by E2F1 and EGF receptor. Mol. Carcinog. 45:10-17 (2006). Nuclear EGFR has also been shown to also interact with STATS to enhance human aurora A gene expression, leading to chromosome instability.

A recent, systemic unbiased approach to identify nuclear EGFR target genes was accomplished using a set of three isogenic glioblastoma cell lines expressing vector control, EGFR, and nuclear entry-defective EGFR (lacking the functional nuclear localization signal within the juxtamembrane region) followed by DNA microarray for over 47,000 gene transcripts. See, Lo, H-W., Cao, X., et al. Cyclooxygenase-2 is a novel transcriptional target of the nuclear EGFR-STAT3 and EGFRvIII-STAT3 signaling axes. Mol. Cancer Res. 8:232-245 (2010). The results indicated 19 potential target genes of nuclear EGFR of which COX-2 was subsequently validated to be a novel transcriptional target of nuclear EGFR. The results further demonstrated that STAT3 greatly synergized with nuclear EGFR to enhance COX-2 gene expression. Importantly, it was demonstrated that nuclear EGFRvIII also activated COX-2 gene expression. The impact of STAT3 on nuclear EGFRvIII-mediated COX-2 expression was found to be only modest, which is in contrast to the significant positive impact of nuclear EGFR-STAT3 complex on COX-2 gene activation. Id. Ongoing efforts are being invested on validating other potential nuclear EGFR target genes that have been identified by the gene expression profiling.

Another mechanism for nuclear EGFR-associated transcriptional regulation was suggested by Huo, et al., (RNA helicase A is a DNA-binding partner for EGFR-mediated transcriptional activation in the nucleus. Proc. Natl. Acad. Sci. USA 107:16125-16130 (2010)) that RNA helicase A serves as a DNA-binding partner for nuclear EGFR. Knockdown of RNA helicase A expression in cancer cells abolished nuclear EGFR binding to its target gene promoters and reduced EGFR-induced gene expression. Interestingly, a more recent study by Jaganathan, et al., (A Functional Nuclear Epidermal Growth Factor Receptor, Src and Stat3 Heteromeric Complex in Pancreatic Cancer Cells. PLoS 1:6 (2011)) showed that EGFR, Src and STAT3 form a heteromeric complex in the nucleus. This nuclear complex is bound to the c-Myc gene, which may contribute to c-Myc gene overexpression in pancreatic cancer cells. Also interesting and indicative of a possible mechanism underlying the ability of nuclear EGFR to regulate gene transcription is the ability of nuclear EGFR to interact with MUC1, which may promote both the accumulation of chromatin-bound EGFR and the significant co-localization of EGFR with phosphorylated RNA polymerase II. See, e.g., Bitler, B. J., Goverdhan, A., Schroeder, J. A. MUC1 regulates nuclear localization and function of the epidermal growth factor receptor. J. Cell Sci. 123:1716-1723 (2010).

HER2 can also be detected in the cell nucleus and activates COX-2 gene expression by binding to HER2-associated sequences. See, e.g., Wang, S. C., Lien, H. C., et al. Binding and transactivation of the COX-2 promoter by nuclear tyrosine kinase receptor ErbB-2. Cancer Cell 6:251-261 (2004). Nuclear HER2 has been shown to associate with STAT3 to upregulate cyclin D1 gene expression. See, e.g., Beguelin, W., Flaque, M. C. D., et al. Progesterone receptor induces ErbB-2 nuclear translocation to promote breast cancer growth via a novel transcriptional effect: ErbB-2 function as a coactivator of Stat3. Mol. Cell. Biol. 30:5456-5472 (2010). This study also showed that progesterone receptor induces HER2 nuclear translocation. Interestingly, a recent report also demonstrated that nuclear HER2 enhanced translation by activating transcription of ribosomal RNA genes. See, e.g., Li, L. Y., Chen, H. Y., et al. Nuclear ErbB2 enhances translation and cell growth by activating transcription of ribosomal RNA genes. Cancer Res. 71:4269-4279 (2011). Taken in sum, these findings indicate that nuclear EGFR and EGFRvIII function as transcriptional regulators that cooperate with their transcriptional co-factors to mediate the expression of a number of important cancer-related genes and thereby, regulate many physiological and pathological processes.

EGFR as a Nuclear Tyrosine Kinase

Evidence to date indicates that nuclear EGFR retains its tyrosine kinase activity. See, e.g., Wang, S. C., Nakajima, Y., et al. Tyrosine phosphorylation controls PCNA function through protein stability. Nat. Cell Biol. 8:1359-1368 (2006). Nuclear EGFR phosphorylates proliferating cell nuclear antigen (PCNA) to promote cell proliferation and DNA repair. Chromatin-bound PCNA protein is phosphorylated on the Tyr²¹¹ residue by nuclear EGFR and becomes stabilized. This important finding raised the possibility that additional nuclear proteins may be phosphorylated by both nuclear EGFR and HER2, and that the functions, stability, and/or intracellular trafficking of these Target molecules may be altered as a consequence of tyrosine phosphorylation. Additional efforts are needed to explore these possibilities.

Nuclear EGFR as a Modulator of DNA Repair

Nuclear EGFR also plays an essential role in DNA repair following radiation therapy. It has been shown that upon radiation therapy induced EGFR nuclear entry, EGFR localized in the nucleus interacts with DNA-dependent protein kinase (DNA-PK), leading to repair of radiation-induced DNA double-strand breaks in bronchial carcinoma cells. A non-steroid anti-inflammatory drug (i.e., celecoxib) has been shown to facilitate tumor cell radiosensitization by inhibiting radiation-induced nuclear EGFR transport and DNA repair. See, e.g., Klaus, H. D., Mayer, C., et al. Celecoxib induced tumor cell radiosensitization by inhibiting radiation induced nuclear EGFR transport and DNArepair: A COX-2 independent mechanism. Int. J. Radiat. Oncol. Biol. Phys. 70:203-212 (2008). This action of celecoxib appears to be independent of its COX-2 inhibitory effect since radiosensitization was correlated with neither COX-2 expression nor prostaglandin E2 levels. Another study further demonstrated that nuclear EGFR is required for tumor resistance to DNA damage induced by the DNA alkylating agent, cisplatin. See, e.g., Hsu, S. C., Miller, S. A., et al. Nuclear EGFR is required for cisplatin resistance and DNA repair. Am. J. Translational Res. 1:249-258 (2009). Collectively, these studies suggest a negative impact of nuclear EGFR on tumor sensitivity to DNA-damaging radiation therapy and anti-cancer alkylating agents. A potential mechanism for nuclear EGFR-mediated tumor resistance to cisplatin has been identified, with cisplatin inducing binding of nuclear EGFR and EGFRvIII to DNA-PK, leading to DNA repair. See, Liccardi, G., Hartley, J. A., et al. EGFR Nuclear Translocation Modulates DNA Repair following Cisplatin and Ionizing Radiation Treatment. Cancer Res. 71:1103-1114 (2011). Similar to EGFR, HER2 nuclear transport can be induced by radiation. Interestingly, Herceptin appears to inhibit radiation-induced HER2 nuclear accumulation, suggesting a potential benefit of combining Herceptin with radiation in treating breast cancer patients with HER2-positive tumors.

Nuclear EGFR may protect normal cells from unwanted DNA damage caused by ultraviolet and γ irradiations. Ultraviolet irradiation has been shown to induce EGFR nuclear translocation in human keratinocytes. The mechanisms for the observed protective effects of nuclear EGFR in normal skin cells are still unclear. However, it has been shown that following irradiation and treatment of the radioprotector Bowman-Birk protease inhibitor, nuclear EGFR is able to associate with p53 and MDC1 protein, both of which are essential for formation of DNA repair foci. See, e.g., Dittmann, K., Mayer, C., et al. The radioprotector Bowman-Birk proteinase inhibitor stimulates DNA repair via epidermal growth factor receptor phosphorylation and nuclear transport. Radiother. Oncol. 86:375-382 (2008). Another radioprotector ophospho-1-tyrosine has been shown to activate PKC-epsilon and to trigger nuclear EGFR import and phosphorylation of DNA-PK, leading to repair of DNA double-strand breaks.

Trafficking of Cell-Surface EGFR to the Nucleus

The mechanisms underlying nuclear transport of EGFR begin with endocytosis, which occurs following ligand-induced activation, as the ligand-bound receptors are internalized through clathrin-coated pits that “pinch-off” from the plasma membrane in a dynamin-dependent manner. See, e.g., Campos, A. C. D., Rodrigues, M. A., et al. Epidermal growth factor receptors destined for the nucleus are internalized via a clathrin-dependent pathway. Biochem. Biophys. Res. Comm. 412:341-346 (2011). After the endocytic vesicle fuses with the early endosome, the internalized EGFR can be: (i) recycled back to the plasma membrane; (ii) sorted to late endosomes and, eventually, to lysosomes for degradation; or (iii) further transported into the nucleus. Playing a crucial role in the first two possible outcomes are the various members of the Rab family GTPases. GTP-bound Rab5, for example, assembles on the membrane of early endosomes and recruits Rab tethering proteins to capture the initial clathrin-coated vesicles that pinch-off from the cell surface. Additionally, Rab4 and Rab11 have been implicated to play a role in mediating the budding of recycling vesicles that return EGFR back to the plasma membrane. Rab7 has also been shown to mediate the flow, and subsequent degradation, of EGFR out of the late endosome.

Early endosomal EGFR destined for the nucleus can undergo transport via several proposed models, each of which is dependent on the interaction between a nuclear localization signal (NLS) within EGFR and importin proteins. See, e.g., Giri, D. K., Ali-Seyed, M., et al. Endosomal transport of ErbB-2: Mechanism for nuclear entry of the cell surface receptor. Mol. Cell. Biol. 25:11005-11018 (2005). Importin-β, either alone or as a heterodimer with importin-α, can bind to NLS of NLS-containing proteins as well as to components of nuclear pore complexes (NPCs), thereby directing these proteins for entry into the nucleus. In the case of EGFR and HER2, a putative NLS has been both identified within the juxtamembrane region and shown to interact with importin-β. For HER2, one proposed model suggests that importin-β associates with the NLS of endosome-bound HER2 and directs it to the nucleus by interacting with the nuclear pore protein Nup358 [117], a constituent of NPCs. Id.

Another proposed model involving EGFR retrograde transport suggests that after early endosomal sorting, ErbB family of proteins destined for the nucleus are trafficked via the Golgi to the ER in COPI-coated vesicles. See, e.g., Wang, Y. N., Wang, H. M., et al. COPI-mediated retrograde trafficking from the Golgi to the ER regulates EGFR nuclear transport. Biochem. Biophys. Res. Comm. 399:498-504 (2010). ER-bound EGFR then interacts with Sec61 translocon, passing through the channel in a similar manner as misfolded proteins undergoing ER-associated protein degradation (ERAD) and entering into the cytosol where it can be picked up by importin-β and transported into the nucleus. See, e.g., Wang, Y. N., Wen, H., et al. The translocon Sec61 beta localized in the inner nuclear membrane transports membrane-embedded EGF receptor to the nucleus. J. Biol. Chem. 285:38720-38729 (2010). This retro-translocation from the ER to the cytosol of full-length EGFR with its hydrophobic transmembrane domain requires the presence of cytosolic chaperone HSP70, which may possibly play a role in solubilizing the receptor and preventing aggregation. Alternatively, ER-bound EGFR may also enter the nucleus via lateral diffusion from the ER membrane through the nuclear pore complex and into the inner nuclear membrane mediated by NLS-importin interaction, as suggested by evidence showing EGFR localized at the inner nuclear membrane and nuclear matrix. Although nuclear export signals have yet to be identified in ErbB family of receptors, the exportin CRM1 has been found to interact with EGFR and HER2, and inhibition of CRM1 using leptomycin B has led to increased accumulation of nuclear EGFR, HER2, and HER3.

Other proteins reported to be involved in EGFR nuclear trafficking include Epstein-Barr virus (EBV) encoded latent membrane protein 1 (see, e.g., Tao, Y., Song, X., et al. Nuclear translocation of EGF receptor regulated by Epstein-Barr virus encoded latent membrane protein 1. Sci. China Life Sci. 47:258-267 (2004)), which was shown to regulate nuclear EGFR translocation in a dose-dependent manner, and PIKfyve kinase, which has been demonstrated to play a role in nuclear transport of EGFR via its interaction with cytoplasmic EGFR upon HB-EGF induced activation. Interestingly, a recent study found that Akt phosphorylation of EGFR is required for both EGFR nuclear translocation and acquisition of Iressa resistance via upregulation of BCRP by nuclear EGFR in breast cancer cells, indicating that advances in our understanding of nuclear EGFR trafficking can lead to further insight into the various approaches to EGFR-targeted therapy.

### EGFR Kinase Experimental Methodologies and Results

### I. Specific Examples and Results of Tavocept-Related Studies on Wild Type and T790M Epidermal Growth Factor Receptor (EGFR) Kinase

Tavocept was shown to inhibit WT EGFR kinase with an IC₅₀ of 24.3±3.7 mM under assay conditions of 10 μM ATP concentration. See, FIG. 1. Assays quantitated ADP produced in reactions where WT EGFR kinase or T790M EGFR kinase was incubated with ATP, polyGT substrate, buffer, and varying concentrations of the test articles using the ADP-glo system by Promega. WT EGFR kinase or T790M EGFR kinase phosphorylated the polyGT substrate using the ATP cofactor and produced ADP. The assays in half area 96-well microtiter plates were 10 μL in volume and contained EGFR (20 ng total or 2 ng/μL), ATP (100 μM), PolyGT substrate (0.2 μg/μL), and the concentrations of the various test articles as indicated; additionally, kinase assay buffer was added to achieve a final volume of 10 μL per assay. Reactions were incubated for 60 minutes at 25° C. in a water bath. Following this 60 minute incubation, reactions were transferred to microplates and the kinase activity was evaluated using the ADP-glo system from Promega (this system monitors ADP produced when EGFR phosphorylates the PolyGT substrate).

At higher ATP concentrations of 100 μM ATP, Tavocept had an IC₅₀ that was 40 mM or higher. These lower and higher ATP concentrations were used in an effort to see if Tavocept had either a competitive or non-competitive inhibitory effect, with respect to ATP binding, on WT EGFR kinase. Typically, in kinase endpoint assays like the Promega ADP-glo assay system, inhibitors are classified as competitive if their IC₅₀ increases notably as the ATP concentration increases. As the ATP concentration was increased from 10 to 100 μM, the IC₅₀ for Tavocept increased; however, we did not determine an IC₅₀ for Tavocept under conditions of 100 micromolar ATP because it was higher than 40 mM which is higher than typical Cmax values for Tavocept in patients. However, the fact that the IC₅₀ was 24.3 mM at 10 μM ATP and then increased to a value greater than 40 mM at 100 μM ATP suggests that the Tavocept effect is “competitive-like” with respect to ATP binding.

Tavocept has been administered at doses as high as 41 g/m² and, physiologically, concentrations of Tavocept as high as 18 mM have been achieved in the clinic. See, e.g., Verschraagen M, Boven E, Zegers I, Hausheer F H, van der Vijgh W J F. Pharmacokinetics of Tavocept (BNP7787) and its metabolite mesna in plasma and ascites: a case report. Cancer Chemother. Pharmacol. 51(6):525-529 (2003). Cmax values in plasma of 10 mM are typical with doses of 18.4 g/m²; therefore, the concentrations of Tavocept required to see an effect on WT-EGFR kinase activity in vitro under 10 μM ATP conditions are physiologically relevant. ATP is often in the millimolar range in vivo (see, e.g., Lu X, Errington J, Chen V J, Curtin N J, Boddy A V, Newell D R. Cellular ATP depletion by LY309887 as a predictor of growth inhibition in human tumor cell lines. Clin. Cancer Res. 6(1):271-277(2000)) and the human body is reported to contain no more than 0.5 moles (˜250 g) of ATP at any time but this supply is constantly and efficiently recycled. In vivo there are many ATP-dependent enzymes that compete for ATP binding, including kinases, synthetases, helicases, membrane transporters and pumps, chaperones, motor proteins, and large protein complexes like the proteasome; the concentrations of ATP used herein are approximations for ATP concentrations that may be available to WT-EGFR kinase in vivo as it competes for ATP with the various other enzymes and proteins that utilize ATP.

For the T790M EGFR kinase, the IC50 value was higher than 40 mM Tavocept. See, Table 13. Given this, and since Tavocept is administered at 18 g/m² and has typical Cmax values of 10 mM, the IC₅₀ values for the T790M EGFR kinase were not determined.

TABLE 13
Summary of Effect of Tavocept on WT EGFR and T790M EGFR

| | Tavocept IC50 (mM) | Tavocept IC50 (mM) |
|---|---|---|
| ATP, μM | on WT EGFR | on T790M EGFR |
| 10 | 24.3 ± 3.7 | >>40 |
| 100 | >40 | >>40 |

### II. Specific Examples and Results of Tavocept-Derived Mesna Disulfide Heteroconjugate Studies on Wild Type and T790M Epidermal Growth Factor Receptor (EGFR) Kinase

A. Tavocept-Derived Mesna Disulfide Heteroconjugates are Very Effective Inhibitors of Wild-Type EGFR Kinase and T790M EGFR Kinase

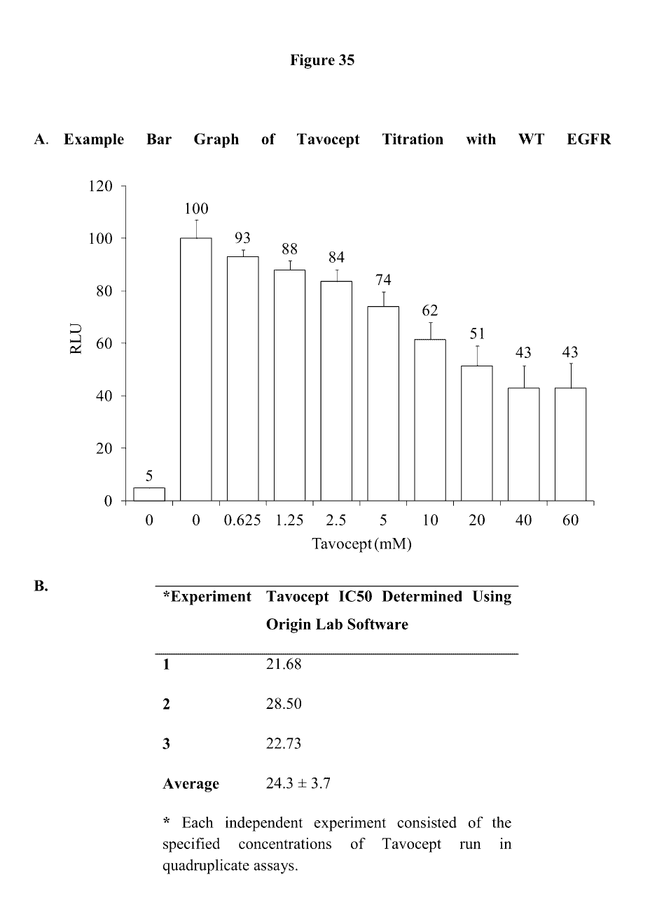

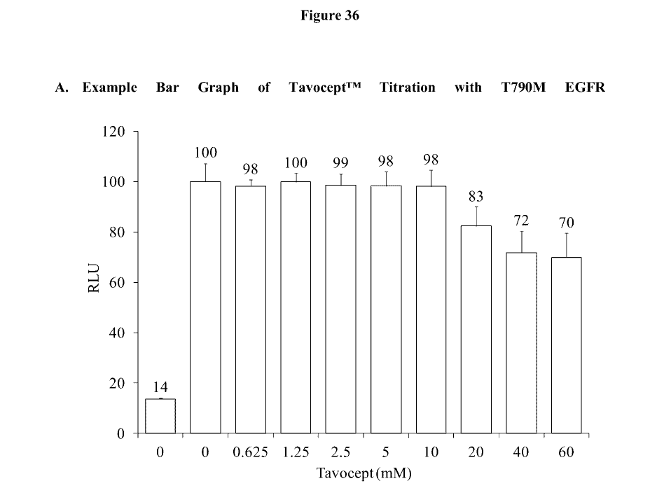

Mesna-glutathione, mesna-cysteine, mesna-cysteinylglycine and mesna-cysteinylglutamate are disulfide heteroconjugates that can be derived from thiol-disulfide exchanges between Tavocept and physiological thiols such as glutathione, cysteine, cysteinylglycine and cysteinylglutamate. See, FIG. 35; Hausheer F H, Kochat H, Parker A R, Ding D, Yao S, Hamilton S E, et al. New approaches to drug discovery and development: a mechanism-based approach to pharmaceutical research and its application to BNP7787, a novel chemoprotective agent. Cancer Chemother. Pharmacol. 52(Suppl. 1):S3-515 (2003); Shanmugarajah D, Ding D, Huang Q, Chen X, Kochat H, Petluru P N, et al. Analysis of BNP7787 thiol-disulfide exchange reactions in phosphate buffer and human plasma using microscale electrochemical high performance liquid chromatography. J. Chromatogr. B. Analyt. Technol. Biomed. Life. Sci. 877(10):857-866 (2009). These Tavocept-derived mesna-disulfide heteroconjugates have the potential to modify EGFR with a moiety that is more sterically bulky than mesna (i.e., cysteine, glutathione, cysteinylglycine and/or cysteinylglutamate). Thus, it has been hypothesized that these Tavocept-related species might be even more potent inhibitors of EGFR kinase.

Mesna-glutathione, mesna-cysteine, mesna-cysteinylglycine and mesna-cysteinylglutamate were all effective inhibitors of both WT EGFR and T790M EGFR kinase with IC₅₀ values that were in the high micromolar to low millimolar ranges (see, Table 14 and Table 15). All of the Tavocept-derived mesna-disulfide heteroconjugates were more effective inhibitors of EGFR kinase (WT and T790M) than Tavocept. When ATP concentrations were varied, the IC₅₀ values of the respective mesna-disulfide heteroconjugates did not appreciably increase, suggesting that the heteroconjugates are non-competitive inhibitors, with respect to ATP. Binding of Tavocept and/or the Tavocept-derived mesna-disulfide heteroconjugates could be either proximal to or distal from the ATP site.

TABLE 14
Summary of IC50 Values for Mesna-Disulfide Heteroconjugates on WT EGFR
Kinase Activity

| | IC50 values for Heteroconjugates on | | | | |
|---|---|---|---|---|---|
| | Wild Type EGFR Kinase Activity | | | | |
| | milliMolar | | | | |
| | | Mesna- | Mesna- | Mesna- | Mesna- |
| ATP, μM | Tavocept | Glutathione | Cysteinylglycine | Cysteinylglutamate | Cysteine |
| 10 | 24.3 ± 3.7 | 0.724 ± 0.22 | 0.445 ± 0.08 | 0.714 ± 0.12 | 2.75 ± 0.8 |
| 100 | ≧40 mM | 0.82 ± 0.24 | 0.37 ± 0.121 | 0.815 ± 0.02 | 2.65 ± 0.11 |
| *250 | N/D | 1.57 | 0.32 | 0.69 | 2.87 |

*250 micromolar ATP experiments were conducted only once and, therefore, error bars are not shown.

TABLE 15
Summary of IC50 Values for Mesna-Disulfide Heteroconjugates on T790M
EGFR Kinase Activity

| | IC50 values for Heteroconjugates on | | | | |
|---|---|---|---|---|---|
| | T790M EGFR Kinase Activity | | | | |
| | (milliMolar values) | | | | |
| | | Mesna- | Mesna- | Mesna- | Mesna- |
| ATP, μM | Tavocept | Glutathione | Cysteinylglycine | Cysteinylglutamate | Cysteine |
| 10 | >40 | 1.28 ± 0.29 | 0.51 ± 0.01 | 1.09 ± 0.19 | 3.76 ± 0.26 |
| 100 | >40 | 1.17 ± 0.01 | 0.41 ± 0.05 | 1.05 ± 0.08 | 3.03 ± 0.28 |

### III. Specific Examples and Results of Tavocept and Tavocept-Derived Mesna Disulfide Heteroconjugate Studies on Erlotinib-Mediated Inhibition of Wild Type and T790M Epidermal Growth Factor Receptor (EGFR) Kinase

Given that NSCLC adenocarcinoma is known to be heterogeneous, and tumors may possibly contain several distinct NSCLC cell populations that have different mutations of proteins like EGFR, (see, e.g., Harris T. Does large scale DNA sequencing of patient and tumor DNA yet provide clinically actionable information? Discov. Med. 10(51):144-150 (2010)) it is possible that by coupling Tavocept or Tavocept-derived mesna disulfide heteroconjugates with Erlotinib, NSCLC tumor cells that contain Erlotinib resistant cells (in this example, cells with T790M EGFR) might respond better.

A. Tavocept Potentiates the Inhibitory Effect of Erlotinib on WT EGFR Activity In Vitro (10 μM ATP)

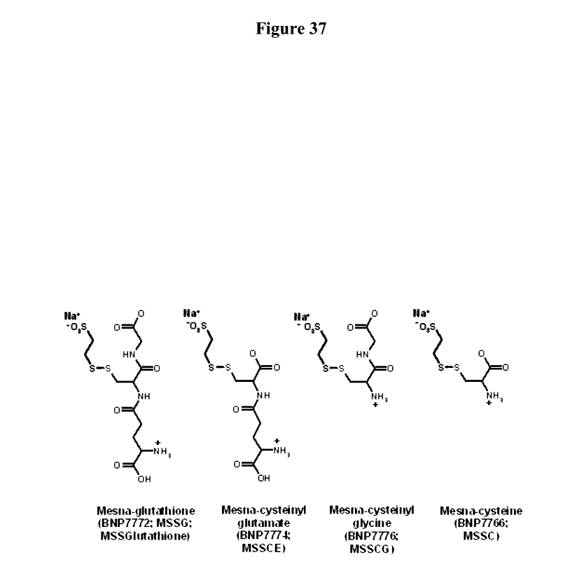

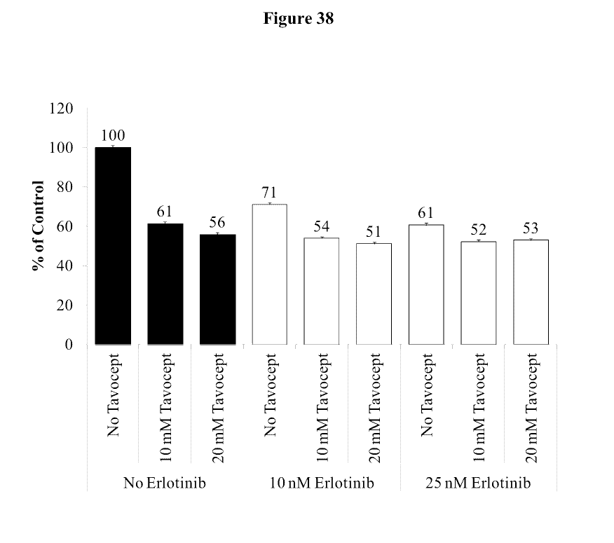

The effect of physiologically achievable concentrations of Tavocept, near the IC₂₅ and IC₅₀ concentrations of Erlotinib under assay conditions with 10 μM ATP, was evaluated. In clinical trials where Erlotinib was administered in a single oral dose of 100 mg to healthy volunteers, Cmax values were 1.39 μg/mL (see, e.g., Ling J, Johnson K A, Miao Z, Rakhit A, Pantze M P, Hamilton M, et al. Metabolism and excretion of Erlotinib, a small molecule inhibitor of epidermal growth factor receptor tyrosine kinase, in healthy male volunteers, Drug Metabolism. Disposition. 34:420-426 (2006)) which corresponds to approximately 3.23 μM; therefore, concentrations of Erlotinib used in these studies were well within physiologically-relevant ranges. As previously discussed, Tavocept has been administered at doses as high as 41 g/m2 and Cmax values in plasma of 10 mM are typical. Tavocept slightly potentiates the inhibitory effect of Erlotinib on WT EGFR kinase at physiologically relevant concentrations of both Tavocept and Erlotinib (see, FIG. 37). Under assay conditions with 10 μM ATP (see, FIG. 38), 10 mM Tavocept in combination with 10 nM Erlotinib (near the IC25 value for Erlotinib, when ATP is 10 μM) resulted in 10% greater inhibition than 10 nM Erlotinib alone; whereas 20 mM Tavocept in combination with 10 nM Erlotinib resulted in 15% greater inhibition than 10 nM Erlotinib alone. Under assay conditions with 10 μM ATP (see, FIG. 38), 10 mM Tavocept in combination with 25 nM Erlotinib (near the IC₅₀ value of Erlotinib) resulted in 9% greater inhibition than 25 nM Erlotinib alone.

In addition, inspection of data in FIG. 38 indicates that Tavocept alone at 10 and 20 mM appeared to be essentially equally effective in inhibiting WT EGFR Kinase Activity in assays with 10 μM ATP, resulting in 39% and 44% inhibition, respectively; whereas, 10 nM and 20 nM Erlotinib alone resulted in 29% and 39% inhibition, respectively.

B. Tavocept Strongly Potentiates the Inhibitory Effect of Erlotinib on T790M EGFR Activity In Vitro (10 μM ATP)

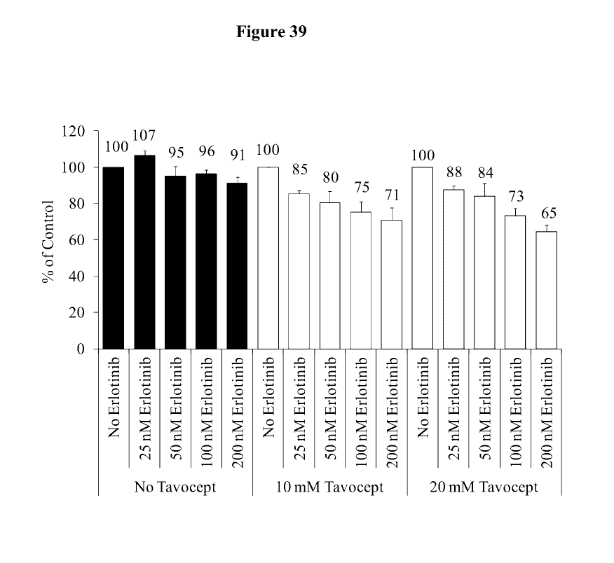

Tavocept strongly potentiated the inhibitory effect of Erlotinib on T790M EGFR kinase at physiologically-relevant concentrations of both Tavocept and Erlotinib (see, FIG. 39). While we will not describe all of the changes in percentage of Control values in FIG. 39, as an example, under assay conditions with 10 μM ATP, 10 mM Tavocept, and 200 nM Erlotinib resulted in approximately 29% inhibition compared to 9% inhibition for 200 nM Erlotinib alone. Additionally, a higher Tavocept concentration (20 mM) gave very similar decreases in percent (%) of control values illustrated in FIG. 39, for example, 20 mM Tavocept in combination with 200 nM Erlotinib resulted in approximately 35% inhibition relative to 9% for 200 nM Erlotinib alone. The effect of Tavocept on Erlotinib-mediated inhibition of the T790M EGFR kinase was found to be dose dependent with respect to Erlotinib.

Assays with T790M EGFR mutant kinase contained 10 μM ATP. The observation of a concentration-dependent, Tavocept-Potentiation of Erlotinib-mediated inhibition of kinase activity (see, FIG. 39) is especially noteworthy for the T790M EGFR kinase for several reasons, including: (i) T790M is resistant to Erlotinib and Erlotinib concentrations as high as 200 nM are relatively ineffective; and (ii) Tavocept alone has essentially no inhibitory activity on T790M EGFR kinase activity (see, FIG. 39).

C. Tavocept-Derived Mesna-Disulfide Heteroconjugates Potentiates the Inhibitory Effect of Erlotinib on WT EGFR Activity In Vitro (10 μM ATP)

As discussed previously, mesna-glutathione (MS SGSH), mesna-cysteine (MSSC), mesna-cysteinylglycine (MS SCG) and mesna-cysteinylglutamate (MS SCE) are disulfide heteroconjugates that can be derived from thiol-disulfide exchanges between Tavocept and physiological thiols such as glutathione, cysteine, cysteinylglycine and cysteinylglutamate. These Tavocept-derived mesna-disulfide heteroconjugates have the potential to modify EGFR with a moiety that is more sterically bulky than mesna (i.e., the non-mesna portion of the disulfide heteroconjugate which is cysteine, glutathione, cysteinylglycine or cysteinylglutamate). Indeed, these heteroconjugates were found to be more potent as individual inhibitors of WT EGFR kinase and inhibition by these inhibitors was not sensitive to ATP concentration.

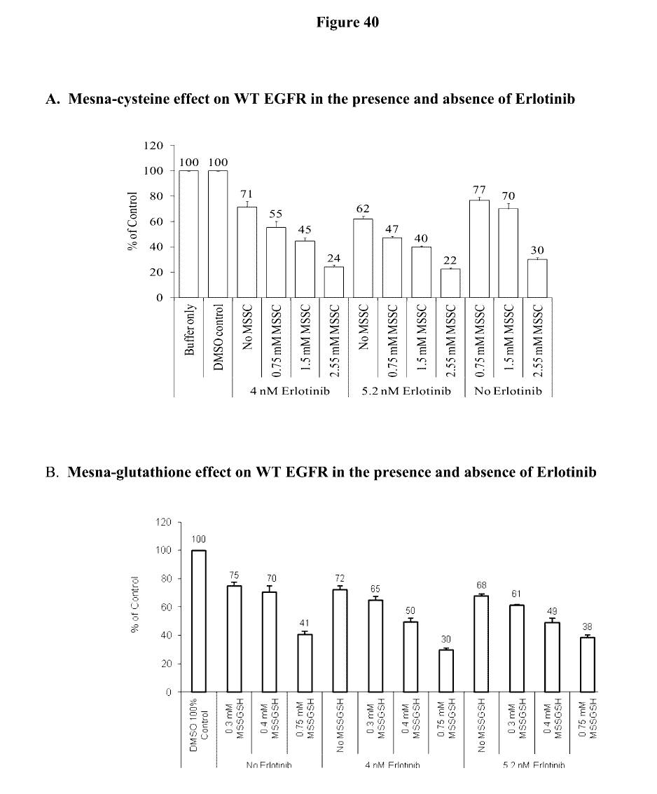

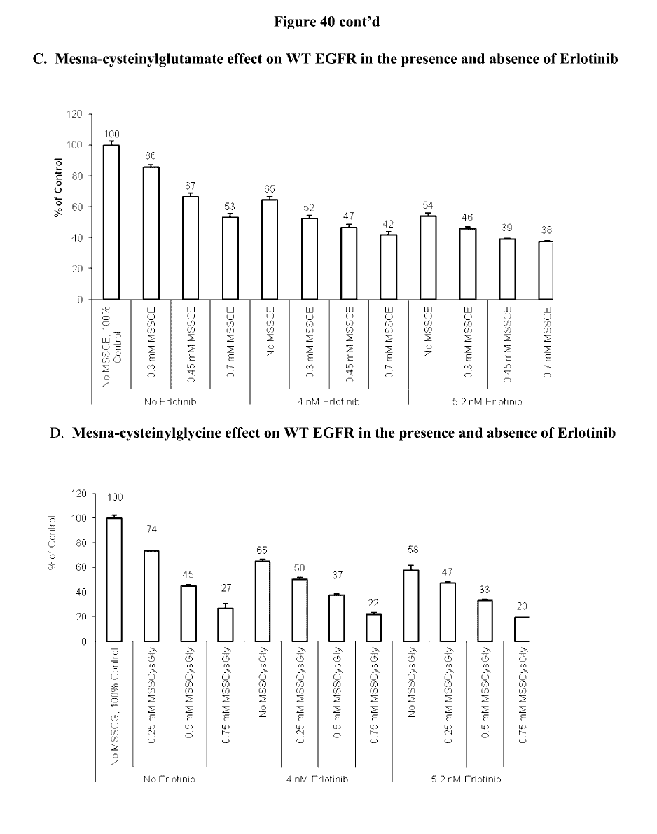

FIG. 40 illustrates that all of the Tavocept-derived mesna-disulfide heteroconjugates were effective at potentiating Erlotinib-mediated inhibition of WT EGFR kinase activity. In the following text, the results from only a few examples will be discussed. For the mesna-cysteine graphical summary (see, FIG. 40, Panel A)—when 4 nM Erlotinib was incubated with mesna-cysteine (MSSC) at 0.75, 1.5, and 2.5 mM, increased inhibition was observed corresponding to 16%, 34%, and 54% more inhibition relative to 4 nM Erlotinib incubated without any MSSC. Similarly for the mesna-cysteinylglycine (MSSCG) graphical summary (see, FIG. 40, Panel B)—when 4 nM Erlotinib was incubated with 0.25, 0.50, and 0.75 mM MSSCG, increased inhibition was observed corresponding to 15%, 28%, and 43% relative to 4 nM Erlotinib incubated without any MSSCG. Further examination of the graphs in FIG. 40, reveals that all of the mesna-disulfide heteroconjugates appear to potentiate the effect of Erlotinib on WT EGFR kinase activity.

D. Tavocept-Related Mesna-Disulfide Heteroconjugates Potentiate the Inhibitory Effect of Erlotinib on WT EGFR Activity In Vitro (100 μM ATP)

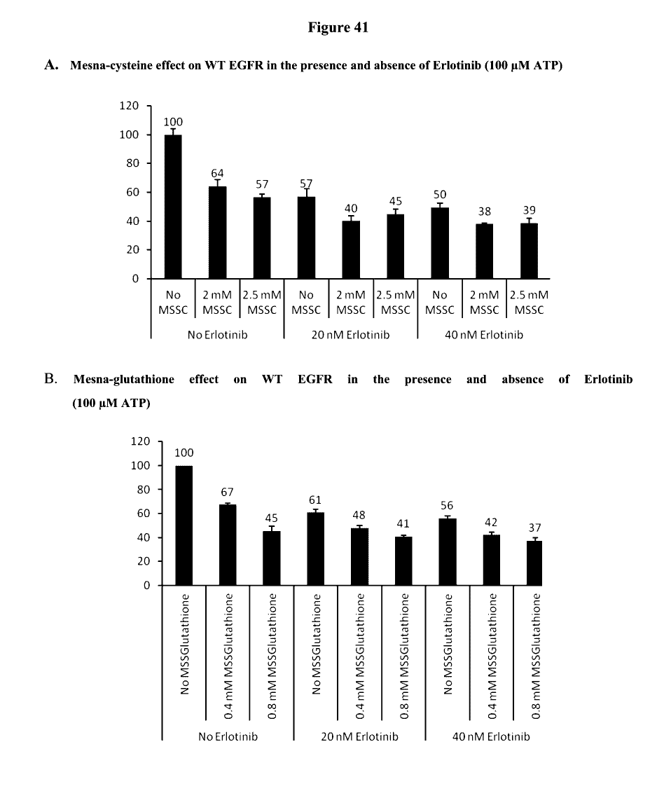

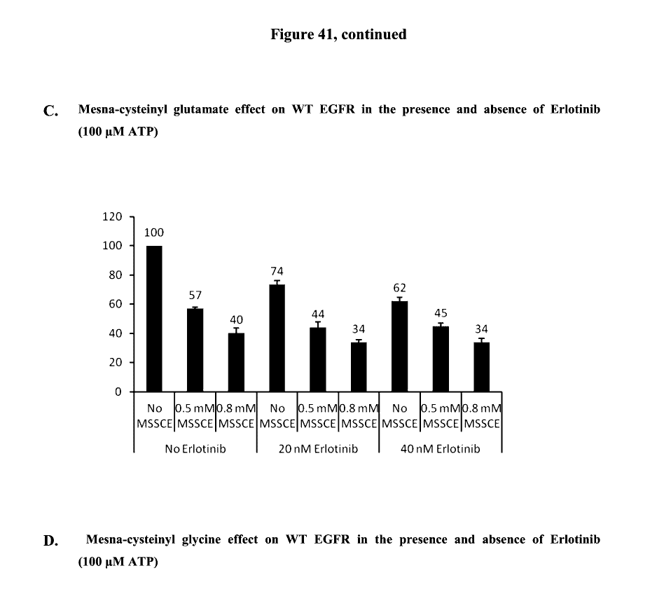

Similar to what was discussed above, potentiation of WT EGFR activity under higher ATP conditions (100 μM ATP) was also observed. See, FIG. 41, Panels A-D. For the mesna-cysteine (MSSC) graphical summary in FIG. 41, Panel A, when 20 nM Erlotinib was incubated with 0.2 and 2.5 mM MSSCG, increased inhibition was observed corresponding to 17% and 15%, respectively, relative to 20 nM Erlotinib incubated without any MSSC. Further examination of the graphs in FIG. 41 reveals that all of the mesna-disulfide heteroconjugates appear to potentiate the effect of Erlotinib on WT EGFR kinase activity under assay conditions of 100 μM ATP.

E. Tavocept-Derived Mesna-Disulfide Heteroconjugates Potentiate the Inhibitory Effect of Erlotinib on T790M EGFR Activity (10 μM and 100 μM ATP)

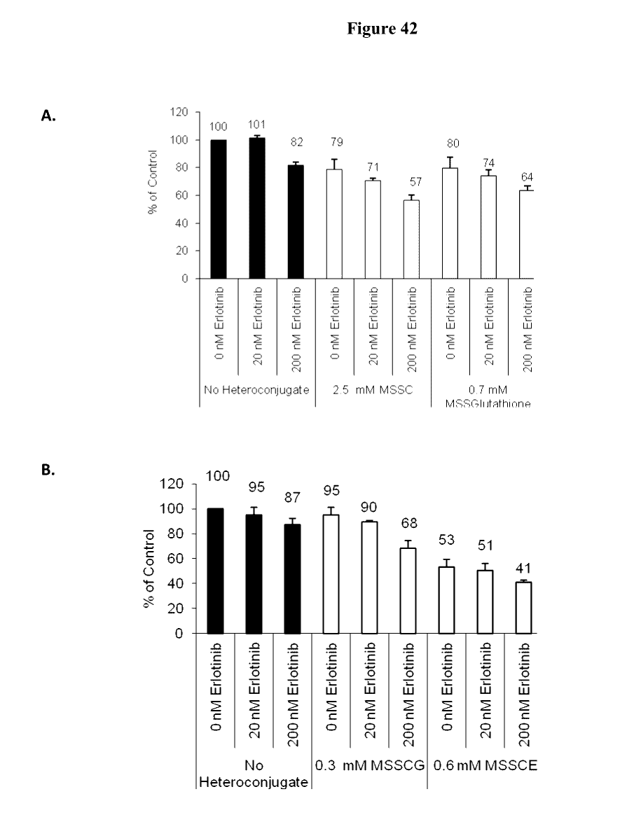

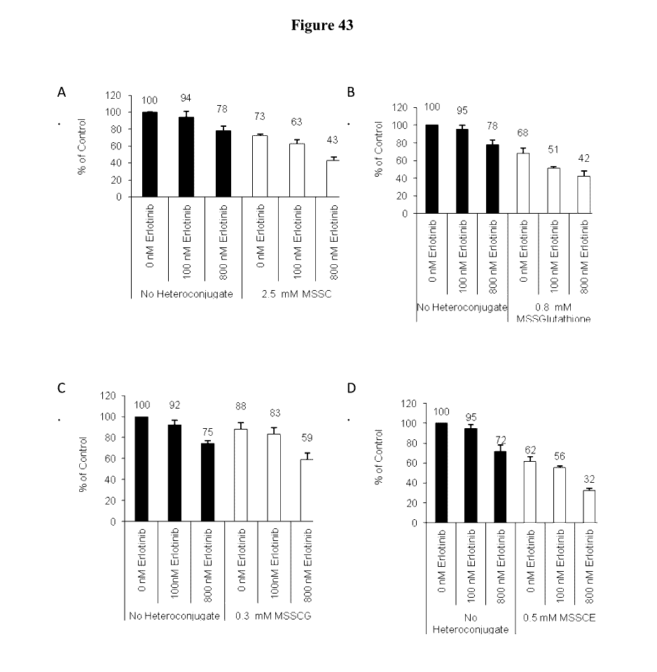

Tavocept-derived mesna-disulfide heteroconjugates were effective at potentiating Erlotinib-mediated inhibition of T790M EGFR kinase activity. FIG. 42 summarizes the data showing the ability of Tavocept-derived mesna-disulfide heteroconjugates to potentiate Erlotinib activity (10 μM ATP). Tavocept-derived mesna-disulfide heteroconjugates were effective at potentiating Erlotinib-mediated inhibition of T790M EGFR kinase activity. FIG. 43 summarizes the data showing the ability of Tavocept-derived mesna-disulfide heteroconjugates to potentiate Erlotinib activity (100 μM ATP).

### IV. Summary of Results of Tavocept and Tavocept Metabolite-Related Studies on Human EGFR Kinase Activity

The results of experiments described above evaluating the effect of Tavocept on WT EGFR and T790M EGFR kinase activity support several conclusions, including:
In assays with 10 μM ATP, Tavocept inhibits WT EGFR kinase with an IC50 value of 24.3±3.7 mM.
In assays with 100 μM ATP, physiological concentrations (<20 mM) of Tavocept did not notably inhibit WT EGFR kinase.
In assays with 10 μM or 100 μM ATP, physiological concentrations (<20 mM) of Tavocept did not notably inhibit T790M EGFR kinase.
In assays with 10 μM ATP, Erlotinib inhibited WT EGFR kinase with an IC50 value of 25.3 mM.
In assays with 100 μM ATP, Erlotinib inhibited WT EGFR kinase with an IC50 value of 131.8 mM.
In assays with 10 μM or 100 μM ATP, concentrations of Erlotinib that were as high as 200 nM did not notably inhibit the Erlotinib-resistant EGFR kinase mutant, T790M EGFR kinase.
In assays with 10 μM ATP, mesna-cysteine inhibited WT EGFR kinase with an IC50 value of 2.75±0.8 mM.
In assays with 100 μM ATP, mesna-cysteine inhibited WT EGFR kinase with an IC50 value of 2.65±0.11 mM.
In assays with 10 μM ATP, mesna-cysteine inhibited T790M EGFR kinase with an IC50 value of 3.76±0.26 mM.
In assays with 100 μM ATP, mesna-cysteine inhibited T790M EGFR kinase with an IC50 value of 3.03±0.28 mM.
In assays with 10 μM ATP, mesna-glutathione inhibited WT EGFR kinase with an IC50 value of 0.724±0.22 mM.
In assays with 100 μM ATP, mesna-glutathione inhibited WT EGFR kinase with an IC50 value of 0.82±0.24 mM.
In assays with 10 μM ATP, mesna-glutathione inhibited T790M EGFR kinase with an IC50 value of 1.28±0.29 mM.
In assays with 100 μM ATP, mesna-glutathione inhibited T790M EGFR kinase with an IC50 value of 1.17±0.01 mM.
In assays with 10 μM ATP, mesna-cysteinylglycine inhibited WT EGFR kinase with an IC50 value of 0.445±0.08 mM.
In assays with 100 μM ATP, mesna-cysteinylglycine inhibited WT EGFR kinase with an IC50 value of 0.37±0.121 mM.
In assays with 10 μM ATP, mesna-cysteinylglycine inhibited T790M EGFR kinase with an IC50 value of 0.51±0.01 mM.
In assays with 100 μM ATP, mesna-cysteinylglycine inhibited T790M EGFR kinase with an IC50 value of 0.41±0.05 mM.
In assays with 10 μM ATP, mesna-cysteinylglutamate inhibited WT EGFR kinase with an IC50 value of 0.714±0.12 mM.
In assays with 100 μM ATP, mesna-cysteinylglutamate inhibited WT EGFR kinase with an IC50 value of 0.815±0.02 mM.
In assays with 10 μM ATP, mesna-cysteinylglutamate inhibited T790M EGFR kinase with an IC50 value of 1.09±0.19 mM.
In assays with 100 μM ATP, mesna-cysteinylglutamate inhibited T790M EGFR kinase with an IC50 value of 1.05±0.08 mM.
In assays where Erlotinib was tested in combination with Tavocept, under 10 μM ATP conditions, Tavocept slightly potentiated Erlotinib inhibition of WT EGFR kinase activity.
In assays where Erlotinib was tested in combination with mesna-cysteine, under 10 μM and 100 μM ATP conditions, mesna-cysteine effectively potentiated Erlotinib inhibition of both WT EGFR and T790M EGFR kinase activity.
In assays where Erlotinib was tested in combination with mesna-glutathione, under 10 μM and 100 μM ATP conditions, mesna-glutathione effectively potentiated Erlotinib inhibition of both WT EGFR and T790M EGFR kinase activity.
In assays where Erlotinib was tested in combination with mesna-cysteinylglycine, under 10 μM and 100 μM ATP conditions, mesna-cysteinylglycine effectively potentiated Erlotinib inhibition of both WT EGFR and T790M EGFR kinase activity.
In assays where Erlotinib was tested in combination with mesna-cysteinylglutamate, under 10 μM and 100 μM ATP conditions, mesna-cysteinylglutamate effectively potentiated Erlotinib inhibition of both WT EGFR and T790M EGFR kinase activity.
Tavocept, and the Tavocept-derived heteroconjugates, modulate the activity of WT EGFR kinase and/or T790M EGFR kinase in vitro. If this occurs in vivo, it could be a contributing mechanism behind survival benefits in NSCLC and other cancer patients with elevated WT EGFR kinase and/or mutated T790M EGFR kinase activity.

(v) Insulin-Like Growth Factor 1 Receptor Kinase

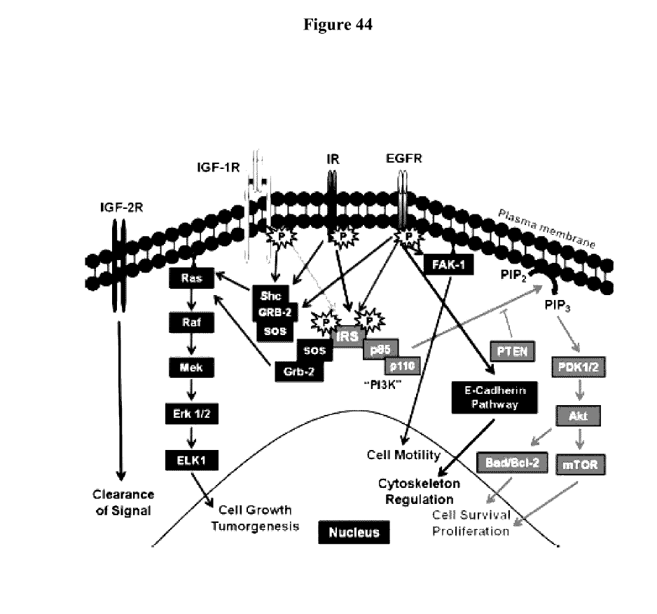

The Insulin Growth Factor 1 Receptor (IGF1R) is a member of the IGF axis, a family of insulin receptor related and insulin growth factor related proteins that are important in endocrine function and cancer. See, e.g., Arnaldez and Helman, Targeting the insulin growth factor receptor 1. Hematol. Oncol. Clin. North. Am. 26(3):527-542 (2012). IGF1R has a high degree of structural similarity to the insulin receptor and modulates cell growth and proliferation through several key proteins including PI3K, IRS, MAPK, JAK/STAT, and others. See, FIG. 44; see, e.g., Fidler, et al, Targeting the insulin-like growth factor receptor pathway in lung cancer: problems and pitfalls. Ther. Adv. Med. Oncol. 4(2):51-60 (2012). IGF1R is important in a variety of cancers including, but not limited to, lung, colon, breast, sarcoma and prostate cancer. See, e.g., Gombos, et al, Clinical Development of Insulin-Like Growth Factor Receptor-1 (IGF1R) Inhibitors: At the Crossroad. Invest. New Drugs 30(6):2433-2442 (2012); Gallagher and LeRoith, IGF, Insulin and Cancer. Endocrinology 152(7):2546-2451 (2011).

Like many receptor tyrosine kinases, IGF1R homodimerizes at the cell membrane and transduces signals through the various signaling pathways. Additionally IGF1R can form heterodimers with other receptors including, but not limited to, the insulin receptor and EGFR2 (HER-2). The heterodimerization with EGFR2 has been proposed to contribute to Trastuzumab resistance in vitro and may have important in vivo implications as well. See, e.g., Maki, Insulin-like Growth Factors and Their Role in Growth, Development, and Cancer. J. Clin. Oncol. 28(33):4985-4995 (2011). IGF1R is the subject of many laboratory studies and more than 60 clinical trials have been initiated to evaluate agents that putatively target IGF1R. See, e.g., Gombos, et al, Clinical Development of Insulin-Like Growth Factor Receptor-1 (IGF1R) Inhibitors: At the Crossroad. Invest. New Drugs 30(6):2433-2442 (2012). However, no compound has yet been approved by the FDA that specifically modulates IGF1R function. Heidegger and co-wokers have suggested that this may be due to the complex and essential role IGF1R has in normal physiology. See, e.g., Heidegger, et al., Targeting the insulin-like growth factor network in cancer therapy. Cancer Biol. Ther. 11(8):701-707 (2011). Studies involving IGF as described herein, were designed to evaluate the effect of Tavocept on IGF1R kinase activity in vitro. Specifically, these studies indicate Tavocept can modestly inhibit the kinase activity of IGF1R in vitro if Tavocept is incubated with IGF1R prior to assaying for kinase activity.

### I. Summary of Tavocept-Related Studies on Insulin-Like Growth Factor 1 Receptor (IGF1R) Kinase

The following experiments were designed to determine whether Tavocept forms a detectable, covalent modification on Insulin-Like Growth Factor Receptor (IGF1R) Kinase. Specifically, these studies indicate Tavocept can modestly inhibit the kinase activity of IGF1R in vitro if Tavocept is incubated with IGF1R prior to assaying for kinase activity.

### II. Specific Example and Summary of Tavocept-Related Studies on Insulin-Like Growth Factor 1 Receptor (IGF1R) Kinase

Tavocept effects on IGF1R kinase activity were evaluated using assays that quantitated ADP produced in reactions where IGF1R was incubated with ATP, IGF1Rtide substrate, buffer and varying concentrations of the test articles using the ADP-glo system by Promega. Prior to initiating the IGF1R kinase assays, IGF1R was incubated with Tavocept—IGF1R kinase phosphorylated the IGF1Rtide substrate using the ATP cofactor and produced ADP. The assays in half area 96-well microtiter plates were 10 μL in volume and contained IGF1R (4 ng total or 0.4 ng/μL), ATP (100 μM), IGF1Rtide substrate (0.4 μg/μL), and the concentrations of the various test articles as indicated. Additionally, kinase assay buffer was added to achieve a final reaction volume of 10 μL per assay. IGF1R kinase reactions were incubated for 60 minutes at 25° C. in a water bath. Following this 60 minute incubation, reactions were transferred to microplates and the kinase activity was evaluated using the ADP-glo system from Promega (this system monitors ADP produced when IGF1R phosphorylates the IGF1Rtide substrate).

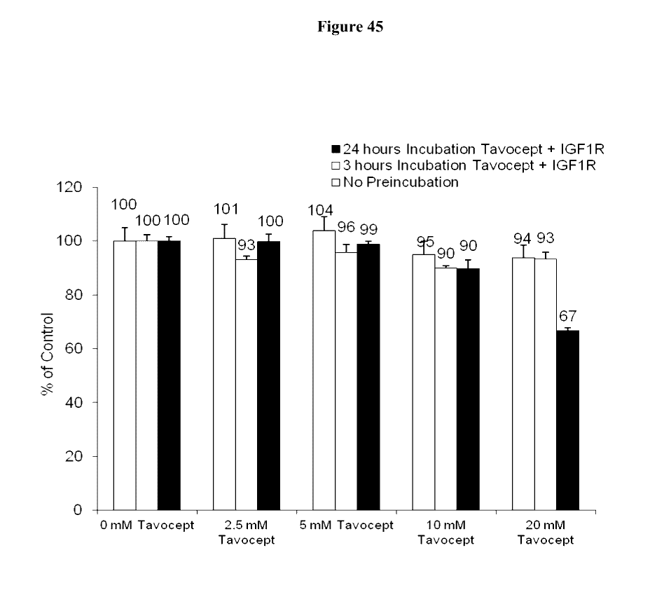

The experiments disclosed herein confirmed that when Tavocept is incubated with IGF1R prior to assaying IGF1R for kinase activity, there is a modest Tavocept effect on IGF1R kinase activity. See, FIG. 45. Specifically, when 20 mM Tavocept is pre-incubated with IGF1R for 24 hours prior to performing the kinase assay, a loss of approximately 33% of activity is seen (see, FIG. 45). Other concentrations and pre-incubation times had losses that were ≦10% of activity. Accordingly, the time dependent effects of Tavocept on IGF1R may indicate that Tavocept have a greater effect if administered prior to any agent that targets IGF1R.

### B. DNA Repair and Replication Enzymes

(i) ERCC1-XPF DNA Repair Endonuclease

DNA excision repair protein ERCC-1 is a protein that in humans is encoded by the ERCC1 gene. The function of the ERCC1 protein is predominantly in nucleotide excision repair (NER) of damaged DNA. In humans DNA repair is mediated through one of five pathways including: (i) nucleotide excision repair (NER); (ii) base excision repair; (iii) mismatch repair; (iv) non-homologous end-joining; and (v) homology directed repair. See, e.g., Jalal, et al., DNA repair: From genome maintenance to biomarker and therapeutic target. Clin. Cancer Res. 17(22):6973-6984 (2011). Nucleotide excision repair (NER) in eukaryotes is initiated by either Global Genome NER (GG-NER) or Transcription Coupled NER (TC-NER) which involve distinct protein complexes, each recognizing damaged DNA. Thereafter, subsequent steps in GG-NER and TC-NER share a final common excision and repair pathway which include the following steps: (i) transcription factor II H (TFIIH) separates the abnormal strand from the normal strand; (ii) xeroderma pigmentosum group G (XPG) cuts 3′ to the damaged DNA: (iii) replication protein A (RPA) protects the “normal”, non-damamged stard; (iv) xeroderma pigmentosum group A (XPA) isolates the damaged segment on the strand to be cut; and (v) ERCC1 and xeroderma pigmentosum group F (XPF) cut 5′ to the damaged DNA. ERCC1 appears to have a crucial role in stabilizing and enhancing the functionality of the XPF endonuclease. The excised single-stranded DNA (approximately 30 nucleotides in length) and the attached NER proteins are excised and removed. DNA polymerases and ligases then fill in the gap left by the excision of the damaged DNA strand using the normal strand as a template.

In mammals, the ERCC1-XPF protein complex also removes non-homologous 3′ tail ends in homologous recombination. The ERCC1-XPF complex is a structure-specific endonuclease involved in the repair of damaged DNA. ERCC1-XPF performs a critical incision step in nucleotide excision repair (NER), and is also involved in the repair of DNA interstrand crosslinks (ICLs) and some double-strand breaks (DSBs). See, e.g., Ahmad, A., Robinson, A., et al. ERCC1-XPF endonuclease facilitates DNA double-strand break repair. Mol. Cell. Biol. 28:5082-5092 (2008). A fraction of ERCC1-XPF is localized at telomeres, where it is implicated in the recombination of telomeric sequences and loss of telomeric overhangs at deprotected chromosome ends. In telomere maintenance, ERCC1-XPF degrades 3′ G-rich overhangs (see, e.g., Kirschner, K., Melton, D. W. Multiple roles of the ERCC1-XPF endonuclease in DNA repair and resistance to anticancer drugs. Anticancer Res. 30:3223-2332 (2010)) and various other related functions (see, e.g., Rahn, J. J., Adair, G. M., Nairn, R. S. Multiple roles of ERCC1-XPF in mammalian interstrand crosslink repair. Environ. Mol. Mutagen. 51:567-581 (2010)).

Deficiency of either ERCC1 or XPF in humans results in a variety of conditions, which include the skin cancer-prone disease xeroderma pigmentosum (XP), a progeroid syndrome of accelerated aging, or cerebro-oculo-facioskeletal syndrome (COFS). These diseases are extremely rare in the general population and therefore mice with low levels of either ERCC1 or XPF have been generated and studied extensively. These murine models clearly illustrate the importance of DNA repair in preventing aging-related tissue degeneration.

### Nucleotide Excision Repair

By way of example, ultraviolet light has the ability to damage DNA in a myriad of manners, most predominantly cyclobutane pyrimidine dimers (CPDs) and (6-4) photoproducts. NER is the only mechanism by which these photodimers can be removed from DNA in human cells, and ERCC1-XPF functions as the nuclease that incises the damaged strand 5′ to the adduct. See, e.g., Tapias, A., Auriol, J., et al. Ordered conformational changes in damaged DNA induced by nucleotide excision repair factors.

J. Biol. Chem. 279:19074-19083 (2004). This incision creates a 3′-terminus that is used as a primer by the replication machinery to replace the excised nucleotides. XPF contains the catalytic activity with its conserved nuclease domain, and ERCC1 is required for binding to DNA. See, e.g., Tsodikov, O. V., Ivanov, D., et al. Structural basis for the recruitment of ERCC1-XPF to nucleotide excision repair complexes by XPA. EMBO J. 26:4768-4776 (2007). Defects in the proteins required for NER can result in xeroderma pigmentosum (XP), trichothiodystrophy (TTD), and Cockayne syndrome (CS), highlighting the importance of DNA repair in preventing UV-induced skin cancer and developmental abnormalities. XP is a disease characterized by extreme photosensitivity and a 10,000-fold increased risk of cutaneous and ocular neoplasms; wherein cells from all of the XP complementation groups (XP-A to XP-G, and XP-V) are hypersensitive to UV radiation. ERCC1-XPF deficient cells are distinct from other XP patient-derived cells because of their extreme sensitivity to chemicals that induce DNA ICLs. An additional critical finding indicates that ERCC1-XPF has functions which are distinct from NER, in that ERCC1 and XPF knockout mice exhibit a much more severe phenotype than XPA null mice (which are completely deficient in NER). See, e.g., Tian, M., Shinkura, R., et al. Growth retardation, early death, and DNA repair defects in mice deficient for the nucleotide excision repair enzyme XPF. Mol. Cell. Biol. 24:1200-1205 (2004).

### Interstrand Crosslink Repair

The mechanism of DNA ICL repair in mammalian cells is not as well defined as NER. In replicating cells, crosslinking agents lead to DSBs created by an endonuclease(s) near the site of stalled replication machinery. In the absence of ERCC1-XPF, replication-dependent crosslink-induced DSBs occur, indicating that ERCC1-XPF cannot be solely responsible for creating these DSBs. See, e.g., Niedernhofer, L. J., Odijk, H., et al. The structurespecific endonuclease Ercc1-Xpf is required to resolve DNA interstrand crosslink-induced double-strand breaks. Mol. Cell. Biol. 24:5776-5787 (2004). Moreover, there is clear evidence that ERCC1-XPF participates in the same mechanism of ICL repair as the Fanconi anemia proteins.

In the absence of ERCC1-XPF, FANCD2 is still mono-ubiquitylated by FANCL, but translocation of FANCD2 to chromatin is impaired. In addition, when FANCD2 is depleted, replication-dependent incisions of ICLs are dramatically reduced. Recently it was demonstrated that XPF binds SLX4 (a related endonuclease) and that this interaction is critical for ICL repair. See, e.g., Munoz, I. M., Hain, K., et al. Coordination of structure-specific nucleases by human SLX4/BTBD12 is required for DNA repair. Mol. Cell 35:116-127 (2009). Fanconi anemia patients, mice deficient in ERCC1-XPF, and Slx4(Btbd12)−/− mice share many spontaneous developmental and degenerative phenotypes, supporting roles for all of these proteins in a common pathway and illustrating the dramatic consequences of failure to repair endogenous ICLs. See, e.g., Crossan, G. P., van der Weyden, L., et al. Disruption of mouse Slx4, a regulator of structure-specific nucleases, phenocopies Fanconi anemia. Nat. Genet. 43:147-152 (2011). Recent reports describe the discovery of biallelic mutations in SLX4 in two patients who exhibited clinical features of Fanconi anemia. See, e.g., Kim, Y., Lach, F. P., et al. Mutations of the SLX4 gene in Fanconi anemia. Nat. Genet. 43:142-146 (2011). Based upon evidence that reintroduction of wild-type SLX4 into the patients' cells rescued sensitivity to crosslinking agents, SLX4 is considered a new complementation group of Fanconi anemia: FANCP.

### Double-Strand Break Repair

Orthologs of ERCC1-XPF in lower eukaryotes such as Arabidopsis thaliana, Drosophila melanogaster, and Saccharomyces cerevisiae play a vital role in the repair of DSBs and meiosis. The two primary mechanisms of DSB repair are non-homologous endjoining (NHEJ) and homologous recombination (HR). Work in budding yeast has contributed tremendously to defining the role of ERCC1-XPF in DSB repair in mammalian cells. Mutation of rad10 or rad1 (the orthologs of ERCC1 and XPF in S. cerevisiae), suppresses HR between sequence repeats. The function of the Rad10-Rad1 nuclease in HR is to remove non-homologous 3′-termini of single-stranded overhangs of broken ends to facilitate single-strand annealing, an error-prone sub-pathway of HR. Like single-strand annealing there is an error prone sub-pathway of NHEJ that utilizes short stretches of homology to join two broken DNA ends, termed micro-homology mediated end joining Rad10-Rad1 also participates in this end joining pathway in yeast. See, e.g., Ma, J. L., Kim, E. M., et al. Yeast Mre11 and Rad1 proteins define a Ku-independent mechanism to repair double-strand breaks lacking overlapping end sequences. Mol. Cell. Biol. 23:8820-8828 (2003).

Yeast Mre11 and Rad1 proteins define a Ku-independent mechanism to repair double-strand breaks lacking overlapping end sequences. Mammalian cells deficient in ERCC1-XPF are moderately sensitive to ionizing radiation (IR), a source of DSBs. Like in yeast, HR and end joining of DSBs is attenuated in ERCC1-XPFdeficient mammalian cells, as the ERCC1-XPF endonuclease is required for efficient single-strand annealing and gene conversion in mammalian cells. See, e.g., Al-Minawi, A. Z., Saleh-Gohari, N., Helleday, T. The ERCC1/XPF endonuclease is required for efficient single-strand annealing and gene conversion in mammalian cells. Nucleic Acids Res. 36:1-9 (2008). Therefore, it is proposed that ERCC1-XPF nuclease facilitates both the HR and NHEJ pathways (single-strand annealing and microhomology-mediated end-joining) but only if the broken DNA ends contain 3′-overhanging unmatched sequences or ends that cannot be used to prime DNA synthesis. See, e.g., Ahmad, A., Robinson, A. R., et al. ERCC1-XPF endonuclease facilitates DNA double-strand break repair. Mol. Cell. Biol. 28:5082-5092 (2008).

### Telomeric Interactions

ERCC1-XPF deficiency is linked with accelerated aging, and telomere shortening is associated with aging, so therefore it was important to understand if the nuclease impacts telomere length or function. Telomeres in humans with mutations in XPF, or ERCC1 knockout mice are not shorter than controls and there is no difference in sister chromatid exchange at telomeres in the absence of ERCC1-XPF. However, ERCC1 co-localizes with TRF2 at telomeres. See, e.g., Zhu, X. D., Niedernhofer, L., et al. ERCC1/XPF removes the 3′ overhang from uncapped telomeres and represses formation of telomeric DNA-containing double minute chromosomes. Mol. Cell 12:1489-1498 (2003). In a TRF2 dominant negative background, ERCC1-XPF deficient cells accumulate telomeric double-minutes. This led to the conclusion that ERCC1-XPF cleaves the G-rich, 3′-overhang, rendering chromosomes vulnerable to end-to-end fusions. Hence the absence of ERCC1-XPF apparently does not have a deleterious impact on telomere length or function. Consistent with that, correction of XP-F cells or overexpression of XPF in normal human cells leads to telomere shortening. See, e.g., Wu, Y., Mitchell, T. R., Zhu, X. D. Human XPF controls TRF2 and telomere length maintenance through distinctive mechanisms. Mech. Ageing Dev. 129:602-610 (2008). Therefore, accelerated aging associated with ERCC1-XPF deficiency is presumed to arise from cellular senescence and cell death and not as a consequence of telomere-dependent replicative senescence.

### Human ERCC1 Mutations

ERCC1 was the first human DNA repair gene cloned. For decades, however, no patients were identified with ERCC1 mutations. Recently, however, a single patient was discovered who had mutations in ERCC1 resulting in severe pre- and post-natal developmental defects. See, Jaspers, N. G., Raams, A., et al. First reported patient with human ERCC1 deficiency has cerebrooculo-facio-skeletal syndrome with a mild defect in nucleotide excision repair and severe developmental failure. Am. J. Hum. Genet. 80:457-466 (2007). The patient, referred to as 165TOR, had severe skeletal defects at birth, including microcephaly, arthrogryposis and rocker-bottom feet. These abnormalities were seen in conjunction with neurological alterations including cerebellar hypoplasia and blunted cortical gyri. The clinical diagnosis was cerebro-oculo-facio-skeletal syndrome, or COFS syndrome (a rare autosomal recessive disorder in which patients undergo rapid neurologic decline). Patients with COFS syndrome are reported to have mutations in genes encoding DNA repair proteins ERCC6/CSB, ERCC5/XPG and ERCC2/XPD. Two mutations were found in the coding region of ERCC1 in patient 165TOR. The maternal allele harbors a C→T transition that converts Gln¹⁵⁸ into an amber translational stop codon. The result is a truncated polypeptide that lacks the entire C-terminal domain, essential for binding XPF. See, e.g., de Laat, W. L., Sijbers, A. M., et al. Mapping of interaction domains between human repair proteins ERCC1 and XPF. Nucleic Acids Res. 26:4146-4152 (1998). The paternal allele has a C→G transversion, resulting in the conversion of Phe²³¹ to leucine. This amino acid falls within the C-terminal tandem helix-hairpin-helix domain of ERCC1, which is critical for binding XPF and is conserved in both invertebrates and mammals. See, Id. While ERCC1 mRNA levels were found to be normal in this patient, the protein levels of ERCC1 and XPF in the nucleus were reduced 4-5-fold. The truncated protein was not detectable by immunoblot. Accordingly, fibroblasts from patient 165TOR had 15% of the normal level of NER (representing a relatively modest defect) suggesting that the missense mutation affects stability of ERCC1-XPF and/or its nuclear localization, but not its enzymatic activity.

A second patient with mutations in ERCC1 was briefly described recently. See, Imoto, K., Boyle, J., et al. Patients with defects in the interacting nucleotide excision repair proteins ERCC1 or XPF show xeroderma pigmentosum with late onset severe neurological degeneration. J. Invest. Dermatol. 127:(Suppl. (92)) (2007). The patient had a nonsense mutation affect amino acid 226, which lies early in the helix-hairping-helix domain necessary for binding XPF. The second allele contains a splicing mutation (IVS6-G→A). The patient displayed neurologic symptoms beginning at age 15 years and died by the age of 37. Neurodegeneration was progressive and severe resulting in dementia and cortical atrophy. The symptoms are very similar to XPF patients with neurologic involvement, thus supporting the conclusion that ERCC1 and XPF function exclusively as a complex in vivo. In conclusion, little is known about regulation of ERCC1-XPF expression, which could be tissue-specific and therefore contribute to heterogeneous phenotypes. Identifying modifier genes, identifying regulators of nuclease expression, and the modeling of additional patient mutations in mice will be essential components in the deciphering of genotype:phenotype correlations.

### Human XPF Deficiency

Humans with mutations in XPF can be classified into two groups based upon the clinical manifestations of their disease. The first, which comprise the majority of XP-F patients, present with mild symptoms of XP (e.g., sun sensitivity, freckling of the skin, and basal or squamous cell carcinomas typically occurring after the second decade of life). This is in contrast to many XP-A and XP-C patients, in which skin cancer occurs even before two years of age. The second group of XP-F patients exhibit neurological deterioration in addition to their XP-like symptoms. There has been one published case of a patient with mutations in XPF with dramatically accelerated aging. The mutation in XPF, its impact on protein expression, function and subcellular localization are all critical determinants in the clinical manifestations. See, e.g., Ahmad, A., Enzlin, J. H., et al. Mislocalization of XPF-ERCC1 nuclease contributes to reduced DNA repair in XP-F patients, PLoS Genet. 6:e1000871 (2010). Of note, all XP-F patients carry a missense mutation in at least one allele, and none of these affect the catalytic domain of the protein. This has led to speculation that ERCC1-XPF is essential for human life. This is supported by the observation that mice homozygous for null alleles of these genes are not viable except in select genetic backgrounds.

The first XPF-deficient human patient was reported in 1979, several years before the XPF gene was identified and cloned. The patient, referred to as XP23OS, was confirmed as XP-F by genetic complementation analysis, and exhibited mild XP symptoms including freckling and photosensitivity. Primary cells from patient XP23OS have only 10% of the normal level of NER as measured by UV-induced unscheduled DNA synthesis (UDS), but only modest sensitivity to UV as measured by clonogenic survival. The seeming discrepancy can be explained by the fact that UDS measures NER that occurs in the first 3 hours following UV irradiation, whereas in a clonogenic survival assay cell growth is measured in the 7-10 days following UV irradiation. Thus XP23OS cells must have low levels of NER, but that is adequate to prevent cell death and replicative senescence given ample time to repair the genome. Furthermore, host cell reactivation of reporter expression following UV damage was only modestly impaired. These results suggest that although the efficiency of NER was impaired in this patient, the pathway must be intact to explain the relatively mild symptoms in this 45-year-old patient. In the years that followed, several patients with XP group F were described, most of them from Japan, having mild to moderate symptoms, similar to patient XP23OS. See, e.g., Norris, P. G., Hawk, J. L., et al. Xeroderma pigmentosum complementation group F in a non-Japanese patient. J. Am. Acad. Dermatol. 18:1185-1188 (1988). The majority of XP-F patients had UV sensitivity and freckling of the skin, but severe ocular and neurological symptoms were rare in the XP-F complementation group. See, e.g., Berneburg, M., Clingen, P. H., et al. The cancer-free phenotype in trichothiodystrophy is unrelated to its repair defect. Cancer Res. 60:431-438 (2000).

A. Specific Examples and Summary of Experimental Results of Tavocept-Related Studies on Excision Repair Cross Complementing Group 1 (ERCC1)

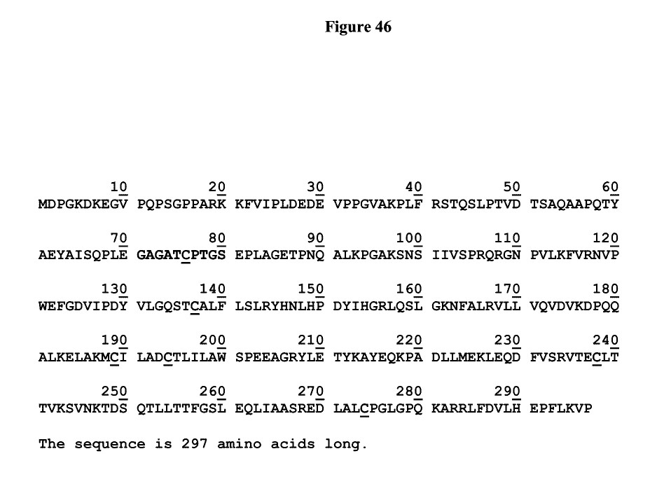

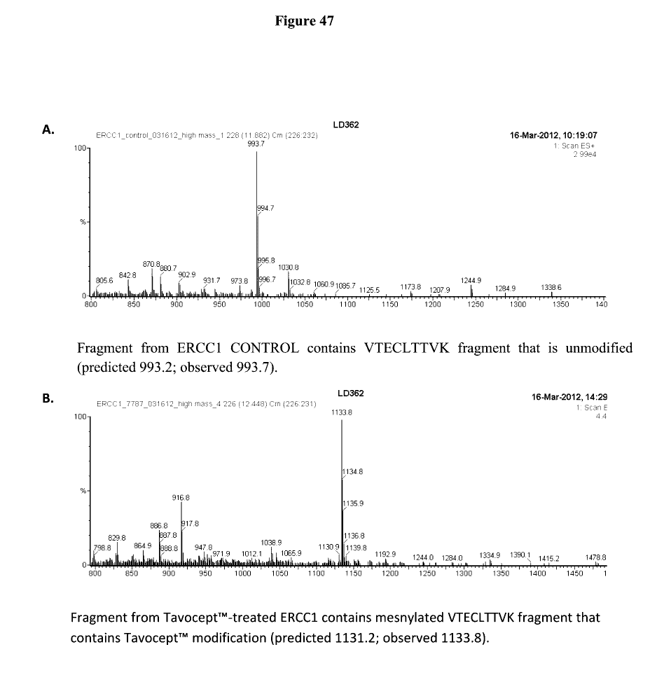

The following experiments were designed to determine whether Tavocept forms a detectable, covalent modification on Excision Repair Cross Complementing Group 1 (ERCC1). Specifically, these studies address whether Tavocept can undergo thiol-disulfide exchange with selected cysteine residues on ERCC1 resulting in formation of a Tavocept-derived mesna-cysteine mixed disulfide. Mass Spectroscopy and peptide digest experiments described in the following sections confirm that Tavocept forms mixed-disulfides with cysteine (Cys) residues of human ERCC1. See, FIG. 46 and FIG. 47, specifically the Tavocept-derived mesna adduct that formed with Cys238 and Cys274.

Recombinant human ERCC1 (1 mg; 27.8 nanomoles, Creative BioMart) was reduced using a vast excess of dithiothreitol (DTT, 100 μl of a 500 mM stock) in ammonium bicarbonate (40 mM, pH 8.0) at 37° C. for 75 minutes (total reaction volume was 900 μL). The DTT was then removed using a NAP10 (G25 Sephadex column; GE Life Sciences) and the DTT-free, reduced protein (750 μL) was incubated for 16 hours with Tavocept (20 mM; 40 μL of a 400 mM stock plus 10 μL buffer) or a control consisting of buffer alone (50 μL) at 30° C. (total reaction volumes of both the Tavocept and the buffer control reactions were 800 μL). Each 800 μl reaction was then chromatographed over a NAP10 column. This step removed unreacted Tavocept and was used for the buffer control simply to ensure that both samples received the same handling/manipulation during the course of the experiment. The elution volume from each NAP10 column was 1.2 mL. After the use of gel filtration to remove excess Tavocept as described above, to the ERCC1 protein (1.2 mL), from the Tavocept reaction and from the control reaction was added 120 μL of the prepared stock Trypsin gold solution (stock was 100 μg of Trypsin Gold in 400 μl of ammonium bicarbonate (40 mM, pH 8.0) and 100 μl of Acetonitrile). Trypsin digestion reactions were incubated for 1 hour at 30° C. and then for 17 hours at room temperature (23° C.). Following the Trypsin digest, the samples were lyophilized to dryness overnight and then resuspended in a volume of approximately 100 μL water and analyzed using LC-MS. A Symmetry C18 HPLC column (Waters, Franklin, Mass.; 3.5 μm; 4.6 mm×75 mm) and a Waters Alliance liquid chromatography system (Waters 2695, Franklin, Mass., USA) coupled to a Micromass single quadropole mass detector (Micromass ZMD, Manchester, UK) were used to analyze fragments from trypsin-digested human ERCC1. The mobile phase contained 0.1% of formic acid throughout the run and the flow rate was 0.35 mL/min. The elution scheme involved the following steps: Step 1: 0 to 3.5 minutes mobile phase was 95% water/5% acetonitrile; Step 2: 3.5 to 20 minutes linear gradient to 10% water/90% acetonitrile; Step 3: 20-30 minutes hold at 10% water/90% acetonitrile; Step 4: 30-40 minutes linear gradient from 10% water/90% acetonitrile to 95% water/5% acetonitrile. Positive-ion electrospray ionization mode (ESI), across the mass ranges of 200-2000 Da, was used. Human ERCC1 contains 6 cysteine residues. See, FIG. 44. Trypsin digestion of human ERCC1 results in formation of up to 30 fragments. Mass Spectroscopy analyses were confined to fragments that were within the range of 200 to 2000 mass units. A summary of 20 of the 30 possible fragments is illustrated in Table 16 (as no fragments of ERCC1 that are smaller than 5 amino acids contain cysteine residues, these small fragments are not included in Table 16).

TABLE 16
Tryptic fragments of human ERCC1 that contain Cysteine residues
(grey highlighted rows)

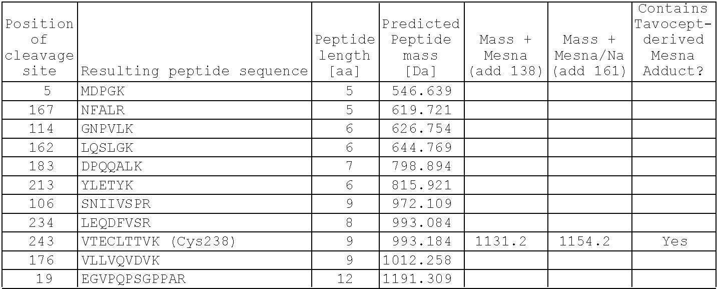

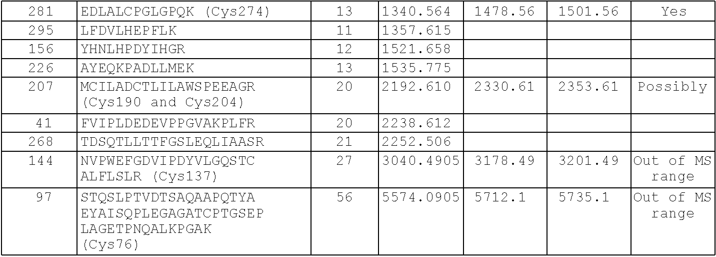

Liquid chromatographic analysis revealed a new peak in the reaction of ERCC1 incubated with Tavocept and Mass Spectroscopy analyses of one of these new peaks revealed the presence of a mesna adduct on Cys238 of ERCC1 in the VTECLTTVK fragment. See, Table 16. In these MS studies, purified recombinant human ERCC1 was incubated for 18 hours with either Tavocept or buffer only (control). Unreacted (free) Tavocept was removed using size exclusion chromatography. Both the control ERCC1 sample and the Tavocept-treated ERCC1 sample were analyzed by liquid chromatography-Mass Spectrometry (LC-MS) for the presence of Tavocept-derived mesna. In the control ERCC1 sample, a MS peak consistent with fragment VTECLTTVK (predicted mass 993.2; fragment contains Cys238), was observed. See, FIG. 47. In the Tavocept-treated ERCC1 sample, a MS peak consistent with xenobiotically modified fragment VTECLTTVK, (predicted mass 1131.2; fragment contains a xenobiotically modified cys238) was observed. See, FIG. 47.

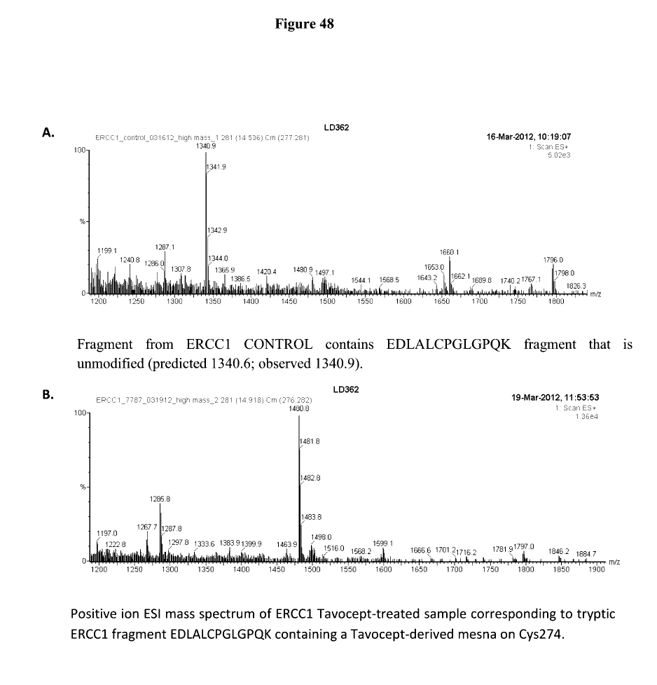

Liquid chromatographic analysis revealed a new peak in the reaction of ERCC1 incubated with Tavocept and mass spectroscopy analyses of one of these new peaks revealed the presence of a mesna adduct on Cys274 of ERCC1 in the EDLALCPGLGPQK fragment. See, Table 16. In the unmodified control ERCC1 sample, a MS peak consistent with fragment EDLALCPGLGPQK (predicted mass 1340.564; fragment contains Cys274), was observed. See, FIG. 48. In the Tavocept-treated ERCC1 sample, a MS peak consistent with xenobiotically modified fragment EDLALCPGLGPQK (predicted mass 1478.6, observed 1480; fragment contains a xenobiotically modified Cys274) was observed. See, FIG. 48.

B. Summary of Studies on Human ERCC1 and Tavocept Interactions
LC-MS indicates Tavocept xenobiotically modifies Human ERCC1 on Cys238.
Modification of Cys238 could disrupt ERCC1 binding to the xeroderma pigmentosum group F (XPF; ERCC4) protein; this dimerization is required for ERCC1-dependent nucleotide excision repair activity.
LC-MS indicates that Tavocept also xenobiotically modifies Human ERCC1 on Cys274.

(ii) Ribonucleotide Reductase

Ribonucleotide reductase (RNR) is a multimeric protein that reduces the 2′ hydroxyl on ribonucleotides to a 2′ hydrogen yielding deoxyribonucleotides that can be utilized in DNA synthesis and DNA repair. See, e.g., Hofer, et al., DNA building blocks: Keeping control of manufacture. Crit. Rev. Biochem. Mol. Biol. 47:50-63 (2012). Human RNR is composed of the subunits M1 (α) and M2 (β or β′) that associate into multimeric forms including a heterodimeric tetramer (α₂β₂) and other complex multimers (αₙ(β₂)ₘ; wherein

n=2, 4, or 6 and m=1 or 3. See, e.g., Wang, et al, Mechanism of inactivation of human ribonucleotide reductase with p53R2 by gemcitabine 5′-disphosphate. Biochemistry 48(49):11612-11621 (2009). The M1 subunit (α subunit; larger subunit) of RNR binds the ribonucleotide substrate and is catalytic while the M2 subunit (β subunit; smaller subunit) contains the diferric tyrosyl radical that is required for catalysis. See, e.g., Wan, et al., Enhanced subunit interactions with gemcitabine-5′-diphosphate inhibit ribonucleotide reductases. Proc. Natl. Acad. Sci. U.S.A. 104(36):14324-14329 (2010); Morandi, Biological agents and gemcitabine in the treatment of breast cancer. Annals Oncol. 17:180-186 (2006); Fairman, et al., Structural basis for allosteric regulation of human ribonucleotide reductase by nucleotide-induced oligomerization. Nat. Struct. Mol. Biol. 18(3):316-322 (2011). RNR is required for de novo DNA synthesis and DNA repair and is, therefore, critical for cell growth and proliferation. See, e.g., Wang, et al, Mechanism of inactivation of human ribonucleotide reductase with p53R2 by gemcitabine 5′-disphosphate. Biochemistry 48(49):11612-11621 (2009).

Unfortunately, only a few drugs have been developed to target human RNR. See, e.g., Wijerathna, et al., Targeting the large subunit of human ribonucleotide reductase for cancer chemotherapy. Pharmaceuticals 4(10):1328-1354 (2010). Gemcitabine is a recently developed small molecule that targets RNR (specifically, Gemcitabine diphosphate targets RNR) and has been used as a single agent and in combination with other agents to treat a range of cancers including non-small cell lung cancer (NSCLC), pancreatic cancer, ovarian cancer and other tumor types. See, e.g., Favaretto, Non-platinum combination of gemcitabine in NSCLC. Annals Oncol. 17:v82-v85 (2006); Long, et al., Overcoming Drug Resistance in Pancreatic Cancer. Expert Opin. Ther. Targets 15(7):817-828 (2011); Matsuo, et al., Overcoming Platinum Resistance in Ovarian Carcinoma. Expert Opin. Investig. Drugs 19(100):1339-1354 (2010). Hydroxyurea is a classical agent targeting RNR and has been used in combination with radiation to treat head and neck cancer and cervical cancer. See, e.g., Chapman and Kinsella, Ribonucleotide reductase inhibitors: A new look at an old target for radiosensitization. Frontiers Oncol. 1:1-6 (2009). RNR has been found to be elevated in some NSCLC patients and development of agents that target and modulate RNR function would be useful in the clinic. See, e.g., Ren, et al., Individualized chemotherapy in advanced NSCLC patients based on mRNA levels of BRCA1 and RRM1. Chin. J. Cancer Res. 24(3):226-231 (2012); Ceppi, et al., ERCC1 and RRM1 gene expressions but not EGFR are predictive of shorter survival in advanced non-small-cell lung cancer treated with cisplatin and gemcitabine. Ann. Oncol. 17(12):1818-1825 (2006); Souglakos, et al., Ribonucleotide reductase subunits M1 and M2 mRNA expression levels and clinical outcome of lung adenocarcinoma patients treated with docetaxel/gemcitabine. Br. J. Cancer 98:1710-1715 (2008).

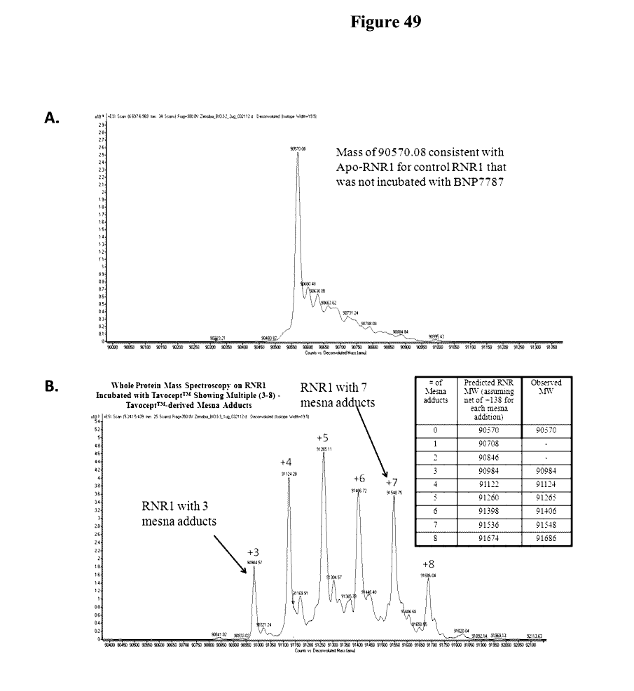

Disclosed herein is whole protein Mass Spectroscopy data that indicates that Tavocept covalently modifies RNR subunit 1 (a subunit) with as many as eight (8) Tavocept-derived mesna adducts on cysteine residues within RNR1. RNR1 contains a total of sixteen (16) cysteine residues and at least five (5) of these cysteine residues are required for catalysis, including: Cys218, Cys429, Cys444, Cys787, and Cys790. It is hypothesized that the Tavocept-derived mesna adducts identified on the RNR1 protein using whole protein Mass Spectroscopy (see, FIG. 49) may alter or modulate RNR activity.

C. Structural Proteins

(i) Tubulin

The structural proteins that comprise the microtubule arrays in vivo are critical for cell division, cell proliferation and a range of other intracellular processes. See, e.g., Harrison, et al., Beyond taxanes: A review of novel agents that target mitotic tubulin and microtubules, kinases, and kinesins. Clin. Adv. Hematol. Oncol. 7:54-64, (2009).

Microtubules consist primarily of α and β tubulin subunits but also contain numerous other microtubule proteins. Oncology drugs that target tubulin have been developed and include drugs in the taxane, epothilone, and vinca alkaloid families. See, e.g., Gascoigne and Taylor, How do anti-mitotic drugs kill cancer cells. J. Cell. Sci. 122:2579-2585 (2009). Agents with the ability to stabilize the tubulin protein within microtubules can result in mitotic arrest and eventually cell death (apoptosis). However, many of the drugs that target tubulin protein and microtubules have side-effects that can be dose-limiting or necessitate the withdrawal of treatment. For example, paclitaxel, a well-known and highly utilized anti-cancer agent exerts its effect primarily by stabilizing tubulin (see, e.g., Xiao, et al., Insights into the mechanism of microtubule stabilization by Taxol, Proc. Natl. Acad. Sci, U.S.A. 103(27):10166-10173 (2006)), but neurotoxicity, manifested primarily as peripheral neuropathy, is a common side effect of taxane-based chemotherapy.

Mechanisms behind chemotherapy-induced peripheral neuropathy (CIPN) are complex, involve damage to the peripheral nerve, and include axonopathy, myelinopathy, and neuronopathy. See, e.g., Lee and Swain, Peripheral neuropathy induced by microtubule-stabilizing agents. J. Clin. Oncol. 24:1633-1642 (2006). Amifostine, glutathione, glutamine/glutamate, calcium/magnesium infusions, neurotrophic factors, NGF, gabapentin, vitamin E, N-acetylcysteine, diethyldithiocarbamate, erythropoietin, and carbamazepine are among the many agents that have been evaluated for use as potential neuroprotective agents. See, e.g., Cavaletti, et al., Neurotoxic effects of antineoplastic drugs: The lesson of pre-clinical studies. Front. Biosci. 13:3506-3524 (2008). However, despite promising results in some clinical trials, no therapy has yet proven effective for the prevention or mitigation of chemotherapy-induced peripheral neuropathy (CIPN), and none of the therapies that have been evaluated thus far have become a standard of care, or have otherwise provided definitive evidence of benefit in the prevention, mitigation, or treatment of CIPN. See, e.g., Parker, et al., BNP7787-mediated modulation of paclitaxel- and cisplatin-induced aberrant microtubule protein polymerization in vitro. Mol. Cancer Ther. 9(9):2558-2567 (2010). Additionally, many of these therapies have adverse side-effects which may limit their utility in patients, and it is presently unknown if there is significant concurrent potential interference with the anti-tumor activity of chemotherapy.

### I. Specific Examples and Summary of Experimental Results of Tavocept-Related Studies on Bovine Tubulin Protein

The following experiments were designed to determine if Tavocept could: (i) modulate microtubule polymerization (α/β-tubulin polymerization in vitro); (ii) modulate the paclitaxel-induced hyperpolymerization of microtubule protein (α-/β-tubulin) in vitro; and/or (iii) modulate the effect of the aquated metabolite of cisplatin, monoaquocisplatin, on microtubule protein (α-/β-tubulin) in vitro. The chemotherapeutic agent paclitaxel is a widely used in the treatment of cancer, including, but not limited to, breast, lung, and ovarian cancer. Paclitaxel is known to modulate the polymerization of microtubule protein (MTP) by specifically targeting α/β-tubulin. See, e.g., Kingston, et al., The Taxol Pharmacophore and the T-Taxol Bridging Principle. Cell Cycle 4:279-289 (2005). Specifically, in the experiments disclosed herein, bovine brain microtubule protein (comprised predominantly of α-/β-tubulin) was purified and used in in vitro microtubule polymerization assays in the presence and absence of Tavocept and its metabolite, mesna, as well as paclitaxel, and the active metabolite of cisplatin, monoaquocisplatin. Data described in the following sections indicates that the Tavocept metabolite, mesna, is able to rescue microtubule protein (i.e., tubulin) from the inactivation mediated by monoaquocisplatin. Further, Tavocept normalizes the hyperpolymerization of tubulin induced by paclitaxel and as a single agent is able to modulate, in a concentration dependent manner, tubulin polymerization in vitro.

In brief, microtubule protein was purified from bovine brain cerebrum as described in the literature. The meninges were removed from fresh bovine cerebrum and the cerebrum was placed into a 1 liter beaker containing ˜300 mL of ice cold buffer A (0.1 M MES, 1 mM EGTA, 0.5 mM MgCl₂, 0.1 mM EDTA, pH 6.5). Grey matter (100 g) was then carefully removed from the cerebrum and placed in a chilled Waring blender to which Buffer A (100 mL), GTP (2.2 mL of 100 mM stock) and β-ME (7 μL of a 14.3 M stock) had been previously added. This heterogeneous mixture was homogenized at high speed in a blender (3×15 seconds). The resulting thick, homogeneous mixture was poured into high-speed polycarbonate centrifuge tubes (26.9 mL volume) and centrifuged at 4° C. for 75 minutes (RCF, =118,747×g). Following centrifugation, the clear, bright red supernatant was poured into a 500 mL graduated cylinder, leaving the large, grey pellet behind. To the bright red supernatant, an equal volume of buffer B (buffer A containing 58.4% glycerol (v/v)), GTP (2.2 mL of 100 mM stock), and β-ME (7 μL of a 14.3 M stock) was added and this mixture was incubated at 37° C. for 30 minutes. During this incubation, 8 mL of layering buffer (layering buffer is a mixture of buffer B (100 mL) and buffer A (30 mL)) was added to clean high-speed polycarbonate centrifuge tubes. The incubated mixture was then carefully layered on top of the layering buffer so as not to disturb the interface between the layering buffer and the clear red supernatant. This two-layer solution was centrifuged at 25° C. for 90 minutes (RCFₐᵥ=196,295×g). The light red supernatant was removed from the clear, colorless, microtubule protein pellet. The pellet was rinsed with room temperature buffer A, so as to remove as much of the residual buffer as possible, and then covered with room temperature buffer B containing β-ME (7 μL of 14.3 M β-ME stock per 100 mL) without any additional GTP beyond that present through the preparation. The microtubule protein pellets were stored at −80° C.

MTP polymerization assays were conducted using standard approaches. The polymerization of α- and β-tubulin subunits into microtubules was monitored at 350 nanometers (OD₃₅₀) on a Cary 100 UV-vis spectrometer using the Cary 100 Kinetics application (Varian Instruments) or on a SpectraMax Plus microtiter UV-vis plate reader using SpectraMax Pro software (Molecular Devices).

Immediately prior to use in assays, frozen, clear microtubule protein pellets were depolymerized and residual chloride ion and GTP were removed by a gel filtration step using a NAP G-25 column. Briefly, the pellets were washed in a chloride-free buffer, designated buffer P (0.1 M PIPES free acid and 1 mM EGTA, pH 6.5). Pellets were resuspended in buffer P (1-2 mL), transferred to a chilled 2 mL Kontes tissue grinder, and incubated on ice for 30 minutes with two homogenizations (2×15 pestle strokes) performed during this time. The microtubule protein was centrifuged at 4° C. for 20 minutes (39,191×g) and the supernatant containing the microtubule protein was transferred to a clean Falcon tube. The microtubule protein supernatant (1.2 mL maximum of a solution that was usually 10-12 mg/mL total protein) was then loaded onto G-25 columns (which had been pre-equilibrated in chloride-free, buffer P) and allowed to fully enter the column. Once the microtubule protein supernatant had fully entered the column, 0.8 mL of chloride-free, buffer P was added. Microtubule protein was then eluted with 3.1 mL of chloride-free, buffer P. Protein concentration was determined by the method of Bradford (see, Bradford M M. A Rapid and Sensitive Method for the Quantitation of Microgram Quantities of Protein Utilizing the Principle of Protein-Dye. Binding. Anal. Biochem. 72:248-254 (1976). By use of SDS-polyacrylamide gel electrophoresis, it was found that the aforementioned microtubule protein preparations were approximately 75% tubulin and 25% microtubule associated proteins (MAPs).

G-25 chromatographed microtubule protein (˜9.7 mg total protein per incubation) was incubated with: (i) buffer only; (ii) mesna only; (iii) cysteine only: (iv) monoaquocisplatin only; (v) mesna plus monoaquocisplatin or (vi) cysteine plus monoaquocisplatin. In general, each sample was 1.25 mL of an approximately 7.8 μg/μL protein sample plus 76 μL of a preincubated mixture of mesna, cysteine, and one of the following reagents: monoaquocisplatin, mesna plus monoaquocisplatin, or cysteine plus monoaquocisplatin. The final assay concentrations of mesna, cysteine or monoaquocisplatin were 200 μM, 200 μM, and 36 μM, respectively. At selected time intervals (e.g., 4, 8, 12, 16 and 24 hours) an aliquot (typically 186 μL) from the various incubation reactions was removed and brought to a 750 μL total volume with buffer P. Final tubulin concentration was approximately 10 μM. From this 750 μL sample, three 196 μL aliquots were transferred to microtiter plate wells and the baseline at OD₃₅₀ was monitored for 1-3 minutes. Microtubule protein polymerization was initiated at 37° C. by addition of GTP (1 mM) and MgSO₄ (0.5 mM) to wells using an automatic pipetman. For these monoaquoplatinum experiments, MgSO₄ was used instead of MgCl₂ to avoid chloride-mediated complications. As previously discussed above, residual chloride ion was removed from solutions for platinum-related experiments using G-25 size exclusion chromatography, as the chloride ion will replace the aquo adduct of monoaquocisplatin reforming cisplatin but monoaquocisplatin is the putative reactive species in vivo. The polymerization reaction was then followed by monitoring the increase in OD₃₅₀ in a microtiter plate format using the SpectraMax Plus plate reader.

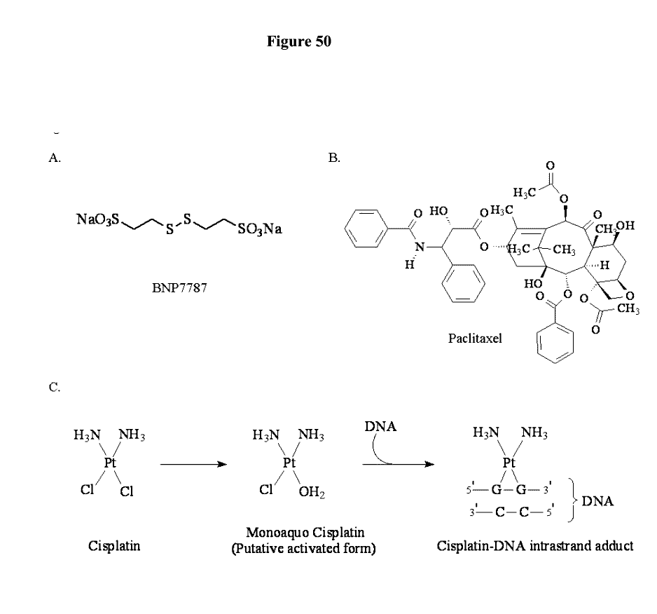

The microtubule protein (10 μM) was preincubated with Tavocept (0-16 mM), mesna (200 μM), or NaCl (32 mM; each mole of Tavocept contains two moles of sodium, NaCl was used as a control; see, FIG. 50) in microcentrifuge tubes, on ice for 20 minutes prior to initiation of microtubule protein assays. The reactions were then transferred from the microcentrifuge tubes to cuvettes or to 96 well plates and polymerization was initiated by the addition of: (i) GTP/MgCl₂ (1 mM/1 mM final concentration); (ii) paclitaxel alone (10 μM); or (iii) Paclitaxel (10 μM), GTP/MgCl₂ (1 mM/1 mM final concentration) and microtubule formation was monitored at OD₃₅₀ using UV-vis spectroscopy.

The microtubule protein was preincubated with Tavocept (6 mM) for 20 minutes in buffer P on ice. After this preincubation, the microtubule protein polymerization reactions were initiated with GTP/MgCl₂ (1 mM/1 mM) or GTP/MgCl₂/paclitaxel

(1 mM/1 mM/6 μM (v/v/v)). Electron micrograph samples were prepared by gently mixing samples of reactions with an equal volume of 50% sucrose in buffer P (0.1 M PIPES, 1 mM EGTA, pH 6.5) and mounting on carbon-coated grids (400 mesh formvar/carbon, Electron Microscopy Sciences). Grids were washed with cytochrome c (1%) and water and stained with uranyl acetate (1%). Electron microscopy was performed using a Philips 208S electron microscope (Philips Instruments) at an accelerating voltage of 60 kV. Micrographs were taken at 36,000× and 7,000× magnifications.

Microtubule protein loses its ability to polymerize over time (referred to as decay) and a decay profile of microtubule protein polymerization is shown in FIG. 50, Panel A. All data from the extended exposures of MTP to monoaquocisplatin with and without mesna or cysteine were normalized by setting the % polymerization values for the pH buffer control to 100% and normalizing the percent polymerization values from the other reactions to this 100% buffer control value (assays were run in triplicate). Decay, or decreases in polymerization, occurs over time most likely because tubulin denatures and precipitates over time. This denatured and/or precipitated tubulin cannot assemble into microtubules and a decrease in total polymerization, as monitored by turbidity at OD₃₅₀, occurs.

The aquated form of cisplatin, monoaquocisplatin, is believed to be the chemotherapeutically active form of cisplatin (see, FIG. 50, Panel C). The equilibrium between cisplatin and monoaquocisplatin is affected by the prevailing chloride concentration in plasma and inside the cell. At high chloride concentrations (e.g., 100 mM in plasma) cisplatin predominates over monoaquocisplatin. However, chloride concentration in most cells is very low (in some cases essentially zero) and once cisplatin enters the cell the low chloride environment facilitates formation of the highly reactive monoaquocisplatin. See, e.g., Reed E. Cisplatin and Analogs. In: Chabner B A, Longo D L, editors. Cancer Chemotherapy and Biotherapy: Principles and Practice. Third Edition. Philadelphia: Lippincott Williams & Wilkins; pp. 447-465 (2001).

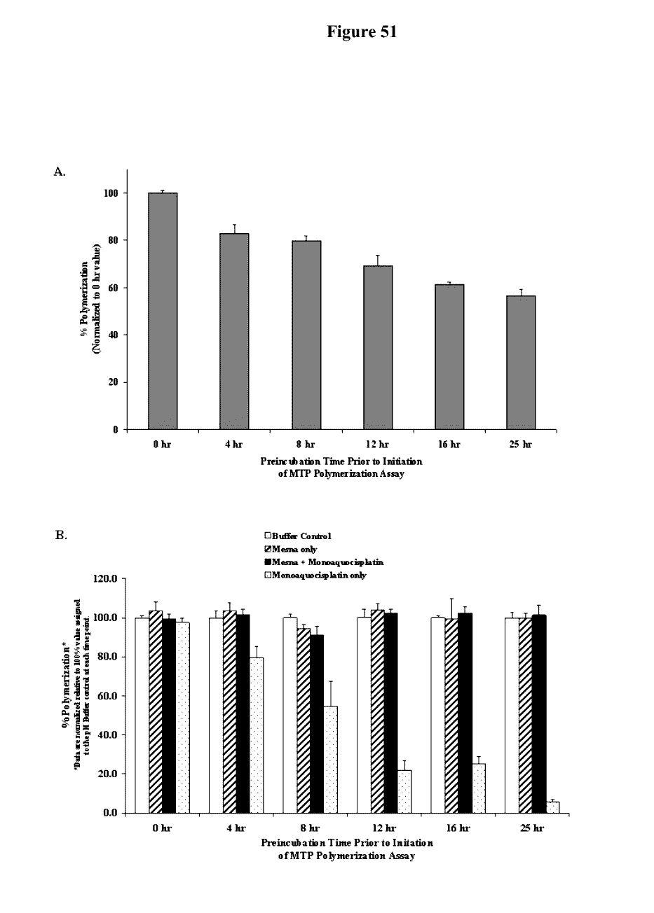

Several groups have reported that extended exposure of microtubule protein to platinum compounds results in the loss of microtubule protein's ability to polymerize into microtubules, a phenomenon called decay. See, e.g. Boekelheide K, Arcila M E, Eveleth J. cis-diamminedichloroplatinum (II) (cisplatin) alters microtubule assembly dynamics. Toxicol. Appl. Pharmacol. 116:146-151 (1992); Peyrot V, Briand C, Crevat A, Braguer D, Chauvet-Monges A M, Sari J C. Action of hydrolyzed cisplatin and some analogs on microtubule protein polymerization in vitro. Cancer Treat. Rep. 67:641-646 (1983). Consistent with these reports, we observed that when monoaquocisplatin was incubated with microtubule protein prior to initiation of MTP polymerization assays, with increasing incubation time there was increased protein denaturation/precipitation. This was reflected in increased background OD₃₅₀ readings (prior to initiation of polymerization assays) for samples from longer incubation times and a smaller net change in OD₃₅₀ when polymerization was initiated. See, FIG. 51, Panel A. This denaturation/precipitation was especially prominent in samples where incubation times prior to initiation of polymerization were ≧8 hours and resulted in a starting OD that was higher (due to denaturation/precipitation of MTP over time) and a smaller total OD change when polymerization was initiated. See, FIG. 51, Panel A. Despite the complications of this decay phenomenon, studies disclosed herein demonstrate that extended exposure of microtubule protein to monoaquocisplatin resulted in the notable and reproducible loss of microtubule protein's ability to polymerize (i.e., loss of ability to polymerize that is beyond the well-documented tubulin decay phenomenon). See, FIG. 51, Panel B. Experiments also demonstrated that short preincubation (30 minutes) of mesna with monoaquocisplatin prevented the monoaquocisplatin-induced loss of microtubule proteins ability to polymerize. See, FIG. 51, Panel B (solid black bars).

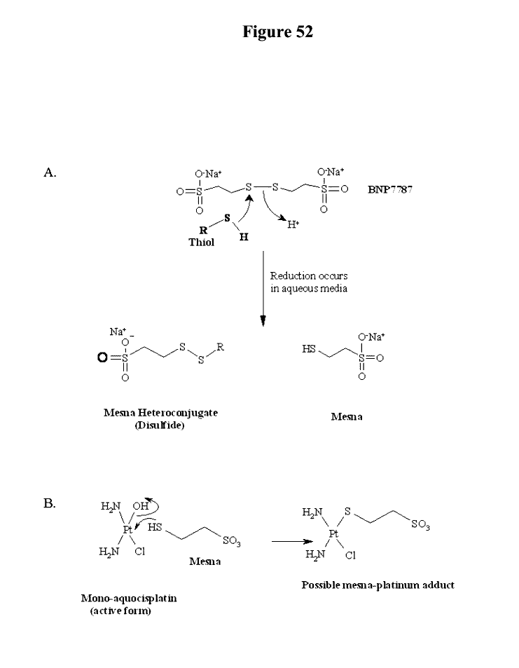

Mesna is a metabolite of Tavocept (see, FIG. 52, Panel A) and is postulated to displace the aquo group from monoaquocisplatin resulting in formation of a platinum-mesna species (see, FIG. 52, Panel A) that is unreactive with MTP. Similar results were observed with the cysteine thiol but were not seen in incubations that contained glutamine or glutamate instead of a thiol like mesna or cysteine. The cysteine effect was interesting, but, unlike Tavocept, administering millimolar concentrations of the cysteine parent disulfide (e.g., cystine) would be difficult due to solubility limits at physiological pH and at millimolar concentrations may well be toxic to humans.

The inhibition of MTP polymerization due to exposure to monoaquocisplatin over time was attributable solely to monoaquocisplatin (4 μL of the low pH monoaquocisplatin solution (pH 3.8) was used in assays with a final volume of 200 μL but the pH of the final 200 μL assay was not changed; additionally low pH solution controls lacking monoaquocisplatin alone had no effect relative to regular pH, control MTP assays). See, FIG. 52, Panel B. It should also be noted that neither mesna nor cysteine alone (in the absence of monoaquocisplatin) had any effect on microtubule protein polymerization.

A. Effect of Tavocept and Mesna on GTP-Catalyzed Microtubule Protein Polymerization

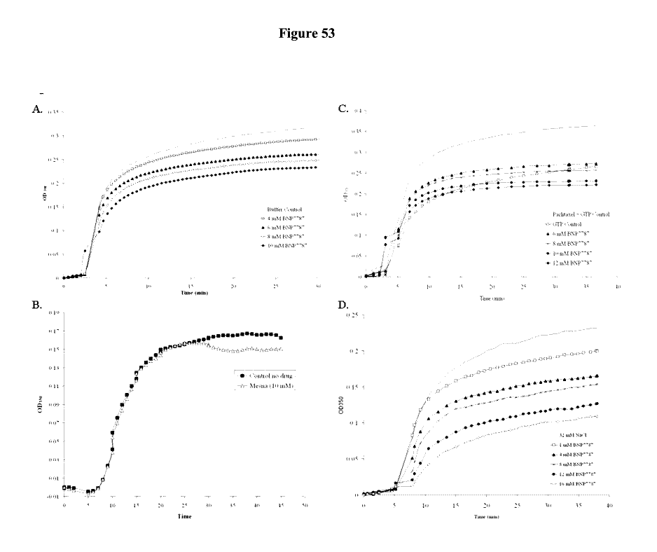

Tavocept (at concentrations of 4, 6, 8, and 10 mM) when preincubated with microtubule protein on ice (for 20 minutes), was found to inhibit GTP-catalyzed polymerization of microtubule protein at 37° C. in a dose-dependent manner. See, FIG. 53, Panel A. These data trends were reproducible using different microtubule protein preparations and different lots of Tavocept. The in vivo metabolite of Tavocept, mesna, did not affect microtubule protein polymerization in an appreciable manner even at very high levels that are not physiologically achievable (e.g., 10 mM). See, FIG. 53, Panel B. At lower mesna concentrations that correspond more closely with peak plasma levels observed in patients, there was no effect on in vitro microtubule protein polymerization (at 41 g/m² Tavocept (˜14 mM Tavocept in plasma) the Cₘₐₓ for mesna was ˜323 μM). Microtubule protein polymerization was unchanged in assays where MTP was preincubated with clinically achievable concentrations of mesna (up to 300 μM) prior to initiation of polymerization and higher concentrations of mesna had no effect. See, FIG. 53, Panel B.

Tavocept (at concentrations of 1, 4, 8, 12 and 16 mM) when preincubated with microtubule protein on ice (for 20 minutes), was found to inhibit paclitaxel-promoted microtubule protein hyperpolymerization in a dose-dependent manner. See, FIG. 53, Panel D. Plasma concentrations equivalent to 10 mM for Tavocept are achieved in clinical trials. Paclitaxel peak plasma concentrations can be as high as 10 μM. In studies performed by the Applicants, paclitaxel (10 μM) was used to achieve a 1:1 (drug:tubulin) subunit ratio and optimal hyperpolymerization effects in the in vitro MTP polymerization assay. Paclitaxel (10 μM) promoted microtubule protein hyperpolymerization was reproducibly inhibited by Tavocept at concentrations of >1 mM. See, FIG. 53, Panel C. While all data herein should be interpreted qualitatively, we observed approximately 50% inhibition of paclitaxel promoted microtubule protein polymerization by Tavocept at concentrations of 12 mM (see, FIG. 53, Panel D) and final polymerization levels of MTP exposed to paclitaxel were essentially equal to controls that lacked Tavocept and paclitaxel (GTP control; see, FIG. 53, Panel C) when ≧6 mM Tavocept was present (see, FIG. 53, Panel C).

Tavocept (at concentrations of 6, 8 10 and 12 mM), when preincubated with microtubule protein on ice (for 20 minutes), was found to inhibit paclitaxel/GTP/MgCl₂-catalyzed microtubule protein hyperpolymerization in a dose dependent manner. See, FIG. 53, Panel C. This dose dependent inhibitory trend was reproducible using different microtubule protein preparations and different lots of Tavocept. When microtubule protein polymerization was initiated by a paclitaxel/GTP/MgCl² mixture (10 μM/1 mM/1 mM, respectively), Tavocept concentrations of 6-8 mM was found to result in a net paclitaxel/GTP/MgCl₂-catalyzed polymerization of microtubule protein equivalent to that observed for a control reaction where polymerization is initiated with GTP/MgCl₂ only. Since 6-10 mM concentrations of Tavocept are pharmacologically achievable, the in vitro inhibitory effects of Tavocept on the microtubule protein polymerization catalyzed by paclitaxel/GTP may be achieved in vivo as well. At lower paclitaxel concentrations, correspondingly lower levels of Tavocept antagonized the effect of paclitaxel on microtubule protein polymerization. Again, these data trends were reproducible using different microtubule protein preparations and different lots of Tavocept. Furthermore, sodium chloride (32 mM; see, FIG. 53, Panel D) did not inhibit microtubule protein polymerization; therefore, the sodium in Tavocept does not exert the inhibitory effect and the inhibitory/protective effects on microtubule protein polymerization under these experimental conditions are attributed solely to Tavocept.

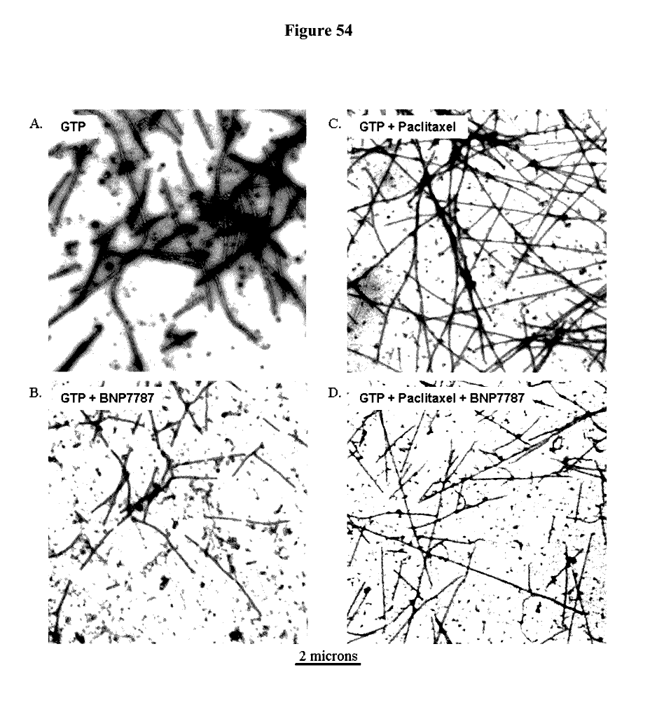

Qualitative evaluation of electron microscopy (EM) grids indicated that Tavocept, preincubated with microtubule protein on ice (for 20 minutes) prior to initiation of microtubule protein polymerization using GTP/MgCl₂, resulted in a reduction in the abundance of microtubules visible in sectors of the grids both in the presence and absence of paclitaxel. See, FIG. 54, Panel A and Panel B. This corresponded well with the decrease in OD₃₅₀ observed in reactions containing Tavocept in the presence and absence of paclitaxel. There were no effects on overall gross microtubule morphology by Tavocept that were discernible using this approach and Tavocept's effect on MTP polymerization in the presence of paclitaxel does not result in the formation of morphologically or structurally aberrant microtubules. However, it appears that when Tavocept and paclitaxel (see, FIG. 54, Panel D) are both present that fewer microtubules (qualitative assessment) are formed compared to when Tavocept is not present (See, FIG. 54, Panel C).

II. Summary of Results from Tavocept-Related Studies on Tubulin

The Tavocept metabolite, mesna, was able to protect against monoaquocisplatin-induced perturbation of MTP polymerization. In contrast, there was no detectable effect of mesna alone on MTP polymerization or on paclitaxel-induced hyperpolymerization of MT).
Tavocept normalizes the well characterized paclitaxel-induced hyperpolymerization of MTP. Since plasma levels of 8 mM Tavocept and higher are pharmacologically achievable at doses of 18.4 g/m² and higher, Tavocept-mediated protection against paclitaxel-induced hyperpolymerization of MTP observed in vitro may potentially occur in patients receiving paclitaxel as well.
Tavocept alone is able to modulate tubulin polymerization in a concentration dependent manner.

C. Oxidoreductases (Redox Enzymes)

Oxidoreductases are enzymes that catalyzes the transfer of electrons from one molecule (i.e., the reductant, also called the hydrogen or electron donor) to another (i.e., the oxidant, also called the hydrogen or electron acceptor). This group of enzymes usually utilizes NADPH or NAD⁺ as cofactors.

(i) Peroxiredoin (Prx)

Peroxiredoxins (Prxs) are a are a ubiquitous family of small (22-27 kDa) non-seleno peroxidases that functions as anti-oxidants and also control cytokine-induced peroxide levels and thereby mediate signal transduction in mammalian cells. Unlike Trx possessing the active double-cysteine region and forming the intramolecular disulfide bond when oxidized, Prx have no such regions; however, the easily oxidized Cys residues present in their structure can form intermolecular disulfide bonds. There are six mammalian isoforms that have been currently identified. See, e.g., Rhee, S., Chae, H., Kim, K. Peroxiredoxins: a historical overview and speculative preview of novel mechanisms and emerging concepts in cell signaling. Free Radical Biol. Med. 38:1543-1552 (2005). Although their individual roles in cellular redox regulation and antioxidant protection are quite distinct, they all catalyze peroxide reduction of H₂O₂, organic hydroperoxides, and peroxynitrite. They are found to be expressed ubiquitously and in high levels, suggesting that they are both an ancient and important enzyme family.

### Mammalian Prx Isoforms

Mammalian cells express six Prx isoforms (Prx 1-6), which can be divided into three subgroups as follow: (i) 2-Cys Prx proteins, which contain both the N- and C-terminal-conserved Cys residues and require both of them for catalytic function; (ii) atypical 2-Cys proteins, which contain only the N-terminal Cys but require one additional, nonconserved Cys residue for catalytic activity; and (iii) 1-Cys Prx proteins, which contain only the N-terminal Cys and require only the conserved one for catalytic function. Four (Prx 1-4) of the six mammalian Prxs belong to the 2-Cys subgroup and have the conserved N- and C-terminal Cys residues that are separated by 121 amino acid residues. Both Prx 1 (NKEF A, PAG, MSP23, OSF3, HBP23) and Prx 2 (NKEF B, Calpromotin, Torin) proteins consist of 199 amino acid residues and exist in cytosol (various alternative names given without reference to peroxidase function are in parentheses). The 257-amino acid sequence of Prx 3 (MER5, SP22) deduced from the cDNA sequence of MER5 is substantially larger than the 195 amino acid residue sequence of SP22, as determined directly by peptide sequencing of SP22 purified from mitochondria of bovine adrenal cortex. The additional 62 residues at the N-terminus were proved to be the mitochondrial-targeting sequence. Prx 4 (AOE372, TRANK) was identified as a protein that interacts with Prx I by the yeast two-hybrid assay. See, e.g., Jin, D. Y.; Chae, H. Z.; et al. Regulatory role for a novel human thioredoxin peroxidase in NF-kappaB activation. J. Biol. Chem. 272:30952-30961 (1997). This protein—protein interaction is probably because a small portion of Prx proteins forms heterodimers. Prx 4 contains the N-terminal signal sequence for secretory proteins and found in culture medium. As demonstrated first with yeast TPx, the N-terminal Cys is oxidized by peroxides to cysteine sulfenic acid, which then reacts with the C-terminal-conserved cysteine of the other subunit to form an intermolecular disulfide. The reduction of the intermolecular disulfide is specific to thioredoxin (Trx) and could not be achieved by glutathione (GSH) or glutaredoxin. Thus, mutant 2-Cys Prx proteins that lack either the N-terminal or C-terminal Cys residues do not exhibit Trx-coupled peroxidase activity. Mammalian cells contain mitochondria-specific Trx and TrxR, suggesting that Prx 3 together with the mitochondria-specifcic Trx and TrxR provide a primary line of defense against H₂O₂ produced by the mitochondrial respiratory chain. See, e.g., Rhee, S., Chae, H., Kim, K. Peroxiredoxins: a historical overview and speculative preview of novel mechanisms and emerging concepts in cell signaling. Free Radical Biol. Med. 38:1543-1552 (2005).

The amino acid sequence identity among the four mammalian 2-Cys (Prx 1 to Prx 4) enzymes is 70%, with the homology being especially marked in the regions surrounding the conserved N- and C-terminal Cys residues. The atypical 2-Cys Prx, Prx5, was identified as the result of a human EST database search with the N-terminal-conserved sequence (KGKYVVLFFYPLDFTFVCP) of the 2-Cys Prx enzymes. The 162-amino acid Prx 5 shares only ˜10% sequence identity with the four mammalian 2-Cys Prx proteins and the sequence surrounding the conserved NH2-terminal Cys (Cys47) (KGKKGVLFGVPGAFTPGCS) is only 52% identical to the search sequence. The C-terminal region of PrxV is smaller than those of 2-Cys Prx enzymes and lacks the conserved sequence containing the C-terminal Cys of the latter enzymes. Both human and mouse Prx 5 sequences contain Cys residues at positions 72 and 151, in addition to the conserved Cys47. However, the sequences surrounding Cys72 and Cys151 are not homologous to those surrounding the C-terminal conserved Cys residue of 2-Cys Prx enzymes, and the distances between Cys₄₇ and these other two Cys residues are substantially smaller than the 121 amino acid residues that separate the two conserved Cys residues in typical 2-Cys Prx enzymes. Cys⁴⁷ is the site of oxidation by peroxides, and the resulting oxidized Cys⁴⁷ reacts with the sulfhydryl group of Cys¹⁵¹ to form a disulfide linkage, which was initially suggested to be intramolecular based on biochemical data. However, recent crystal structures indicate that oxidation of Prx 5 first gives rise to two intermolecular disulfide bonds, which might then rearrange to form intramolecular disulfides. See, e.g., Evrard, C.; Capron, A. et al. Crystal structure of a dimeric oxidized form of human peroxiredoxin 5. J. Mol. Biol. 337:1079-1090 (2004). This is possible because the two disulfide bonds of the oxidized dimer are very close to one another. The disulfide formed by Prx 5 is reduced by Trx, but not by glutaredoxin or GSH. Although only the N-terminal Cys residue is conserved in Prx 5, it is designated as 2-Cys Prx enzyme because its function is dependent on two Cys residues. Prx 5 is localized intracellularly to cytosol, mitochondria, and peroxisomes.

The full-length cDNA (ORF06) for a human 1-Cys Prx, also termed Prx 6, was identified without any reference to peroxidase activity as the result of a sequencing project with human myeloid cell cDNA. Upon exposure to H₂O₂, the N-terminal Cys-SH of Prx 6, which corresponds to Cys47 of human Prx 6, is readily oxidized. However, the resulting Cys-SOH does not form a disulfide because of the unavailability of another Cys-SH nearby. In addition to the Cys⁴⁷ of human Prx 6, some 1-Cys Prx members contain other Cys residues, such as Cys91 of the human enzyme. However, neither Cys91 itself nor the sequence surrounding this residue is conserved among the 1-Cys Prx members. The Cys-SOH of oxidized 1-Cys Prx can be reduced by non-physiological thiols such as DTT. The identity of its redox partner is not yet clear. GSH has been suggested to be the physiological donor for 1-Cys Prx. However, several laboratories have failed to detect GSH-supported peroxidase activity of 1-Cys-Prx. Prx 6 is a cytosolic enzyme.

### Prx Involvement in Oxidative Stress

Although the catalytic activity of Prx towards H₂O₂ (10⁵-10⁶/M/sec) is lower than that of glutathione peroxidase (10⁸/M/sec) and catalase (10⁶/M/sec), they play an important role in detoxification of H₂O₂. Reduction of H₂O₂ by all Prx isoforms passes through formation of sulfenic acid (Cys-SOH) due to oxidation of SH-group of the Cys residue; however, the mechanism of the peroxidase reaction slightly differs in the different Prx isoforms. The typical 2-Cys Prx 1-Prx4 isoforms are homodimers, and their interaction with H₂O₂ leads to formation of sulfenic acid, which can participate in formation of inter-peptide disulfide bond reduced by thioredoxin (Trx). A similar mechanism was ascertained for Prx 5, but the latter is a monomer, and the intramolecular disulfide bond is formed between Cys47 and Cys151. See, e.g., Fujii, J., Ikeda, Y. Redox Rep. 7:123-130 (2002). Prx 1-Prx 5 use thioredoxin (Trx) as a donor of electrons; whereas Prx 6 uses GSH. Moreover, Prx 6 reduces phospholipid hydroperoxides and exhibits activity of phospholipase A₂. See, e.g., Manevich, Y., Fisher, A B. Peroxiredoxin 6 reduces phospholipid hydroperoxides and exhibits activity of phospholipase A₂. Free Radical Biol. Med. 38:1422-1432 (2005). The mechanism of H₂O₂ reduction by Prx 6 includes oxidation of the active Cys⁴⁷ into sulfenic acid followed by its reduction to disulfide by means of S-glutathionylation if heterodimerization of Prx 6 with glutathione transferase P1-1 takes place. The disulfide formed is further non-enzymatically reduced by GSH to restore the functional activity of Prx 6. See, e.g., Manevich, Y., Feinstein, S., Fisher, A. B. Proc. Natl. Acad. Sci. USA 101:3780-3785 (2004).

Since H₂O₂ can rapidly transform into highly toxic reactive oxygen species (ROS), such as O₂⁻ radicals, elevation of the levels of ROS can lead to development of oxidative stress causing deleterious physiological effects, including but not limited to: (i) DNA breakage; (ii) linkages in protein molecules; and (iii) activation of lipid peroxidation. A physiological role of Prx associated with enzymatic degradation of H₂O₂ is particularly significant in erythrocytes, in which these enzymes are ranked second or third place in overall cellular protein content.

An important role of Prx in defense against oxidative stress was demonstrated in a series of studies with knockout of genes corresponding to Prx. Hemolytic anemia, characterized by hemoglobin instability developed, in PRDX1 gene knockout mice. See, e.g., Neumann, C. A., Krause, D. S., et al. Nature 424:561-565 (2003). In PRDX2 gene knockout mice, a significant decrease of lifespan was also accompanied by development of anemia. In both cases, the knockout of the corresponding gene caused a significant elevation of ROS in erythrocytes. The PRDX6 gene knockout mice were characterized by low survival, high level of protein oxidation, and significant injury of kidneys, liver, and lungs. It should be noted that in this case the expression of antioxidant enzymes, such as catalase, glutathione peroxidase, and Mn-SOD did not differ from that in wild-type mice. The results of these studies suggest that function of Prx 6 cannot be compensated by expression of other genes. See, e.g., Wang, X., Phelan, S. A., et al. J. Biol. Chem. 278:25179-25190 (2003).

Nonetheless, H₂O₂ not only contributes to the development of oxidative stress, but at low concentrations it can play a role of secondary messenger involved in intracellular transmission of signals from various surface receptors. H₂O₂ produced with the action of extracellular signals is rapidly eliminated after accomplishment of its function. According to this paradigm, Prx can regulate pathways of cellular signal transduction by control over the level of H₂O₂. See, e.g., Rhee, S. G., Chang, T. S., et al. J. Am. Soc. Nephrol. 14:S211-S215 (2003). In fact, it was found that overexpression of the PRDX1 and PRDX2 genes in transfected cells led to decrease in the level of intracellular H₂O₂ caused by epidermal growth factor and inhibited H₂O₂- and TNFα-dependent activation of the NF-κB transcription factor. It has been shown on the embryonic fibroblast cell culture that overexpression of the PRDX2 gene causes a clear modification of H₂O₂-dependent activation of JNK and p38 kinases in response to TNFα. The authors concluded that Prx can complement effects of other antioxidant enzymes as a modulator of intracellular redox-dependent signaling cascades. See, e.g., Kang, S. W., Chang, T. S., et al. J. Biol. Chem. 279:2535-2543 (2004). Similar results were obtained for the TNFα-dependent activation of the AP-1 transcription factor, which decreased with overexpression of the PRDX2 gene in transfected endothelial cell culture. In thyroid cell culture, overexpression of the PRDX1 and PRDX2 genes eliminated H₂O₂ (whose level was significantly increased under the action of thyrotropin) and protected the cells from H₂O₂-induced apoptosis. See, e.g., Kim, H., Lee, T. H., et al. J. Biol. Chem. 275:18266-18270 (2000).

Studies on crystalline structure of Prx have shown that two functionally active Cys residues act as potential cellular sensor systems determining the role of H₂O₂ either as toxic oxidant or signaling molecule. See, e.g., Wood, Z. A., Poole, L. B., Karplus, P. A. Science 300:650-653 (2003). A model has been proposed in which sensitivity of peroxiredoxins to H₂O₂ correlates with structural changes of these proteins. This model supposes that high intracellular level of Prx with two functionally active Cys residues can retain low level of H₂O₂ in quiescent cells. Alternatively, when the level of H₂O₂ increases (e.g., in cells treated with TNFα) oxidation of redox-sensitive Cys residues reduces their peroxidase activity, and the high level of also concomitantly H₂O₂ activates distinct cellular redox-dependent signaling pathways.

Additionally, there is recent evidence suggests that 2-Cys peroxiredoxins are more than just “simple” peroxidases. This hypothesis has been discussed elegantly in recent review articles, regarding the over-oxidation of the protonated thiolate peroxidatic cysteine and post-translational modification of Prxs as processes initiating a mechanistic switch from peroxidase to chaperon function. See, e.g., Hall, A., Parsonage, D., et al. Redox-dependent dynamics of a dual thioredoxin fold protein: evolution of specialized folds. Biochemistry 48:5984-5993 (2009); Barranco-Medina, S., Lazaro, J. J., Dietz, K. J. The oligomeric conformation of peroxiredoxins links redox state to function. FEBS Lett. 583:1809-1816 (2009). The process of over-oxidation of the peroxidatic cysteine (CP) occurs during catalysis in the presence of thioredoxin (Trx), thus rendering the sulfenic moiety to sulfinic acid, which can be reduced by sulfiredoxin (Srx). However, further oxidation to sulfonic acid is believed to promote Pdx degradation or, as recently shown, the formation of oligomeric peroxidase-inactive chaperones with questionable H₂O₂-scavenging capacity. See, e.g., Lim, J. C., Choi, H. I., et al. Irreversible oxidation of the activesite cysteine of peroxiredoxin to cysteine sulfonic acid for enhanced molecular chaperone activity. J. Biol. Chem. 283:28873-28880 (2008). In the light of these aforementioned functions, as well as the fact that Pdx-1 has recently been shown to interact directly with signaling molecules, there is a distinct possibility that H₂O₂ regulates signaling in the cell in a temporal and spatial fashion via oxidization of Prx 1.

### Prx Expression, Cellular Localization, and Activity

The expression of genes encoding different Prx isoforms has cellular, tissue, and organ specificity. Prx 1 is the most widely represented and highly expressed member of the peroxiredoxin family in virtually all organs and tissues of mice and humans, both in normal tissues and malignant tumors. See, e.g., Li, B., Ishii, T., et al., J. Biol. Chem. 277:12418-12422 (2002). In particular, it should be noted that the PRDX1 gene is widely expressed in various areas of the central and peripheral nervous system with expression specificity depending on the cell type. High expression of the PRDX4 gene is characteristic of liver, testes, ovaries, and muscles, whereas low expression is observed in small intestine, placenta, lung, kidney, spleen, and thymus.

Bast and co-workers found Prx 1 and Prx 2 in pancreatic β-cells of the islets of Langerhans, whereas expression of their genes was absent in the α-cells. See, Bast, A., Wolf, G., Oberbaumer, I., Walther, R. Diabetologia 45:867-876 (2002). Differing expression patterns of genes encoding Prx isoforms have been found in lungs and bronchi. Moderate or high levels of Prx 1, Prx 3, Prx 5, and Prx 6 are found in bronchial epithelial cells, mainly Prx 5 and Prx 6 in alveolar epithelial cells, and Prx 1 and Prx 6 in alveolar macrophages. See, Kinnula, V. L., Lehtonen, S., et al. Thorax 57:157-164 (2002). It should be noted that the contribution of Prx 6 to the antioxidant defense system of the mammalian upper respiratory tract is up to 75%, so in acute inflammatory processes application of Prx6 significantly diminishes the tissue regeneration time. See, Chuchalin, A. G., Novoselov, V. I., et al. Respir. Med. 97:147-151 (2003).

Prx is present in all subcellular compartments, with some specificity of various isoform gene expression being observed. See, e.g., Wood, Z. A., Schroder, E., et al. Trends Biochem. Sci. 28:32-40 (2003). In intracellular organelles, Prx 1 is most widely represented. In addition to Prx 1, Prx 5 is found in cytoplasm, peroxisomes, mitochondria, and nuclei; whereas other isoforms have more restricted subcellular localization. In particular, Prx 2 is present both in the nucleus and cytoplasm, secreted Prx 4 in cytoplasm and lysosomes, Prx 3 in mitochondria, and Prx 6 in cytoplasm.

Regulation of expression of Prx-encoding genes can occur both on the level of transcription and due to post-translational modification. A variety of factors stimulating oxidative stress in murine macrophages influences expression of the PRDX1 gene. See, e.g., Immenschuh, S., Baumgart-Vogy, E. Antioxid. Redox Signal. 7:768-777 (2005). It was found in all cases that induction of expression of this gene was observed together with expression of stress-inducible gene HO-1, whose product is heme oxygenase-1, the rate-limiting enzyme of heme degradation. See, e.g., Otterbein, L. E., Choi, A. M. Am. J. Physiol. Lung Cell. Mol. Physiol. 279:L1029-L1037 (2000). A parallel induction of PRDX1 and HO-1 gene expression was found in smooth muscle vessel cell culture under the action of oxidized low-density lipoproteins and in experiments in vivo in ischemic loci of rat brain. A concerted induction of the PRDX1 and HO-1 genes seems to be a common adaptive response of cells as a defense against oxidative stress. Moreover, the stress-induced induction of gene expression was also marked for other Prx isoforms (e.g., Prx 2 and Prx 6). See, e.g., Kim, H. S., Manevich, Y., et al. Am. J. Physiol. Lung Cell. Mol. Physiol. 285:L363-L369 (2003).

The Nrf2 transcription factor plays the leading role in regulation of the PRDX1 gene expression by electrophilic and ROS-producing agents. See, e.g., Nguyen, T., Sherratt, P. J., Pickett, C. B. Annu. Rev. Pharmacol. Toxicol. 43:233-260 (2003). This finding is supported by data on the absence of expression of this gene under the effect of stress-inducing factors in NRF2 knockout mice. Although Nrf2 is a key regulator of PRDX1 gene expression, various data point to involvement of other transcription factors in regulation of this gene. In particular, expression on the PRDX1 gene in culture of rat macrophages occurs via an AP-1-dependent mechanism when 12-O-tetradecanoylphorbol-13-acetate (TPA) is added. Protein kinase C and Ras protein activating the p38 MAPK-signaling cascade are also involved in this process and PKCδ has been shown to participate in post-translational induction of Prx1. See, e.g., Hess, A., Wijayanti, N., et al. J. Biol. Chem. 278:45419-45434 (2003). Additionally, in macrophage cultures lipopolysaccharides have been demonstrated to induce expression of the PRDX1 gene via the NO-dependent signaling cascade, possibly by means of induction of iNOS. The regulatory role of the NO-dependent signaling pathway was also discovered from the study of the mechanism of induction of PRDX1 and PRDX2 gene expression in pancreatic cell culture. See, e.g., Bast, A., Wolf, G., Oberbaumer, I., Walther, R. Diabetologia 45:867-876 (2002).

The activity of Prx can be modified by post-translational mechanisms, such as phosphorylation, redox-dependent oligomerization, proteolysis, and ligand binding. Phosphorylation of Prx 1, Prx 2, Prx 3, and Prx 4 at Thr amino acid residues by Cdc2 (a cyclin-dependent kinase) has been found to inhibit their peroxidase activities. The mechanism of this inhibition can be explained as a negative modulating effect of negatively-charged phosphate group on the Prx active center through electrostatic interaction. See, e.g., Chang, T. S., Jeong, W., et al. J. Biol. Chem. 277:25370-25376 (2002). Prx can also form dimers and decamers upon change in ionic strength and at low pH values. Activation of Prx oligomerization is evoked by a change in the state of the redox-active disulfide center. A direct functional connection between the redox state and oligomerization has been established for Prx in bacteria. Moreover, a restricted proteolysis of typical double-cysteine Prx from the C-end elevates their resistance to oxidation and subsequently to inhibition of peroxidase activity. See, e.g., Koo, K. H., Lee, S., et al. Arch. Biochem. Biophys. 397:312-318 (2002). The Prx activity can also change due to the noncovalent binding with ligands (e.g., heme and cyclophilin A); wherein the binding of heme to Prx 1 appreciably decreased its activity and the binding of cyclophilin A increased the peroxidase activity of Prx 6. Therefore, in general, the post-translational modifications of Prx result in structural and associated functional changes, which seem to have functional significance for these enzymes as regulators of cellular redox homeostasis.

### Prx Involvement in the Cell Cycle and Cell Proliferation

It is well known that the production of reactive oxygen species (ROS), such as O₂⁻ radicals and cellular redox state play an important role in regulation of the cell cycle and cell proliferation (see, e.g., Sauer, H., Wartenberg, M., Hescheler, J. Cell. Physiol. Biochem. 11:173-186 (2001)) and that antioxidant enzymes, such as glutathione peroxidase and Mn-SOD, are also involved in cell cycle regulation with an increase in ROS production causing an acceleration the cell cycle in fibroblast culture. See, e.g., Oberley, T. D. Am. J. Pathol. 160:403-408 (2002). Similarly, it was also shown in embryonic murine fibroblasts that the cellular level of ROS correlates with the cell cycle time; wherein overexpression of the SOD2 gene inhibits cell proliferation. See, e.g., Li, N., Oberley, T. D. J. Cell. Physiol. 177:148-160 (1998).

The association of Prx 1 with cell proliferation dates from early studies. In particular, it was shown that expression of the PRDX1 gene was appreciably higher in Ras-transfected epithelial cells compared with the wild-type cells. See, e.g., Prosperi, M. T., Ferbus, D., et al. J. Biol. Chem. 268:11050-11056 (1993). Moreover, it was found that Prx 1 interacts with c-Abl and c-Myc protein kinases playing an important role in regulation of cell proliferation. See, e.g., Wen, S.-T., VanEtten, R. A. Genes Dev. 11:2456-2467 (1997). Prx 1 has also been shown to be capable of regulating the tyrosine kinase activity of c-Abl (by binding with its third structural domain), which leads to restriction of the transforming ability of c-Abl. See, Id. Accordingly, it has been hypothysized that the reversible binding of Prx 1 with c-Abl can serve as a key cell cycle regulator. Prx 1 is also capable of binding with c-Myc via the c-Myc-transactivating domain (see, e.g., Mu, Z. M., Yin, X. Y., Prochownik, E. V. J. Biol. Chem. 277:43175-43184 (2002)), with a decrease in expression of a series of genes specific for activity of c-Myc being observed in the case of over-expression of the PRDX1 gene.

As previously noted, Prxs can be specifically phosphorylated at the Thr⁹⁰ residue via the Cdc2 cyclin-dependent kinase, which leads to decrease of the enzyme activity. See, e.g., Chang, T. S., Jeong, W., et al. J. Biol. Chem. 277:25370-25376 (2002). Prx 1 phosphorylation occurs during mitosis rather than in interphase. Phosphorylation of Prxs are believed to play an important role of “switch” in the acceleration of the cell cycle in response to elevation of H₂O₂ levels. See, Id. In addition, Prxs (like other antioxidant enzymes, such as Mn-SOD), have been shown to inhibit proliferation of various tumor cells. See, e.g., Oberley, T. D. Am. J. Pathol. 160:403-408 (2002). Thus, progression of malignant tumors such as lymphomas, sarcomas, and carcinomas is observed in PRDX1 knockout mice. See, e.g., Neumann, C. A., Krause, D. S., et al. Nature 424:561-565 (2003). Accordingly, Prxs are thought to play a role in tumor suppression.

As cell cycle development and apoptosis are related processes, disturbance of the regulation of Cdc2-kinase activity (i.e., phosphorylation) in mammalian cells can result in initiation of apoptosis. See, e.g., Gu, L., Zheng, H., et al. Biochem. Biophys. Res. Commun. 302:384-391 (2003). By way of non-limiting example, it is known that one of the cytokines responsible for inducing ROS production during intracellular signal transmission is TNFα, which induces apoptosis by binding with the death-domain of the TNFα receptor. See, e.g., Chen, G., Goeddel, D. V. Science 296:1634-1635 (2002). In this process, TNFα activates the NF-κB transcription factor involved in redox-dependent gene regulation. See, e.g., Thannickal, V. J., Fanburg, B. L. Am. J. Physiol. Lung Cell. Mol. Physiol. 279:L1005-L1028 (2000). It was found that over-expression of the PRDX2 gene inhibits NF-κB activation after stimulation of cells with H₂O₂ (see, e.g., Kang, S. W., Chae, H. Z., et al. J. Biol. Chem. 273:6297-6302 (1998)), and overexpression of this gene in Molt-4 leukemia cells has a protective effect against ceramide- or etoposide-induced apoptosis (see, e.g., Schreck, R., Rieber, P., Baeuerle, P. A. EMBO J. 10 2247-2258 (1991)). Prx 2 prevents the “leakage” of cytochrome c out of mitochondria and inhibits lipid peroxidation. Interestingly, the over-expression of the PRDX1 gene also has a protective effect on cells exposed to peroxides (see, e.g., Chu, S. H., Lee-Kang, J., et al. Pharmacology 69:12-19 (2003)) and PRDX1 over-expression can suppress the induction of apoptosis and enhance cell resistance to radiation via inhibition of the JNK kinase activity (see, e.g., Kim, Y. J., Lee, W. S., et al. Cancer Res. 66:7136-7142 (2006)); wherein Prx 1 directly binds with the GSTP/JNK complex to markedly increase its stability. Based upon the aforementioned data, one can conclude that the elevation of peroxiredoxin expression inhibits apoptosis, enhances antioxidant effect, and regulates cell proliferation.

I. Specific Examples and Experimental Results for Peroxiredoxin 1

Disclosed herein is data from functional assays which illustrate that Tavocept inhibits the activity of Prx 1 in vitro, and LC MS data that illustrates that Prx 1 is modified by Tavocept on cysteine173 (Cys173) and cysteine 52 (Cys52). Additionally, novel X-ray crystallographic data is disclosed that unequivocally characterize, at an atomic level, the interactions between Tavocept and human Prx 4 and identify a covalent Tavocept-derived mesna mixed disulfide on human Prx 4 at cysteine 124 (Cys124). A second Tavocept-derived mesna mixed disulfide is believed to form at cysteine 245 (Cys245) but due to disorder in the X-ray structure at this site, this cannot be unequivocally confirmed.

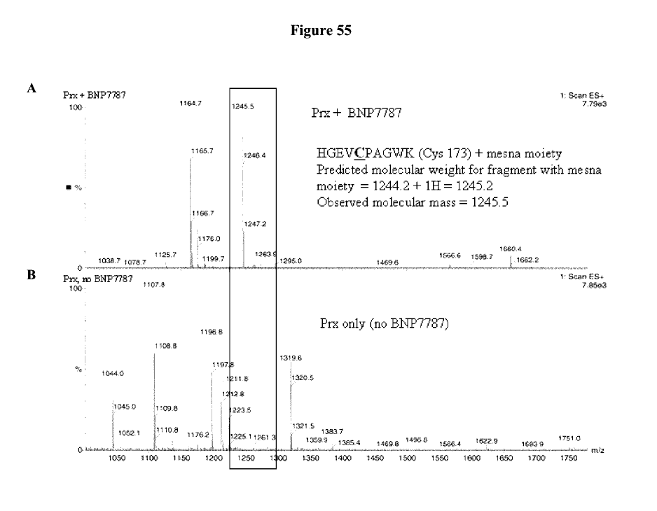

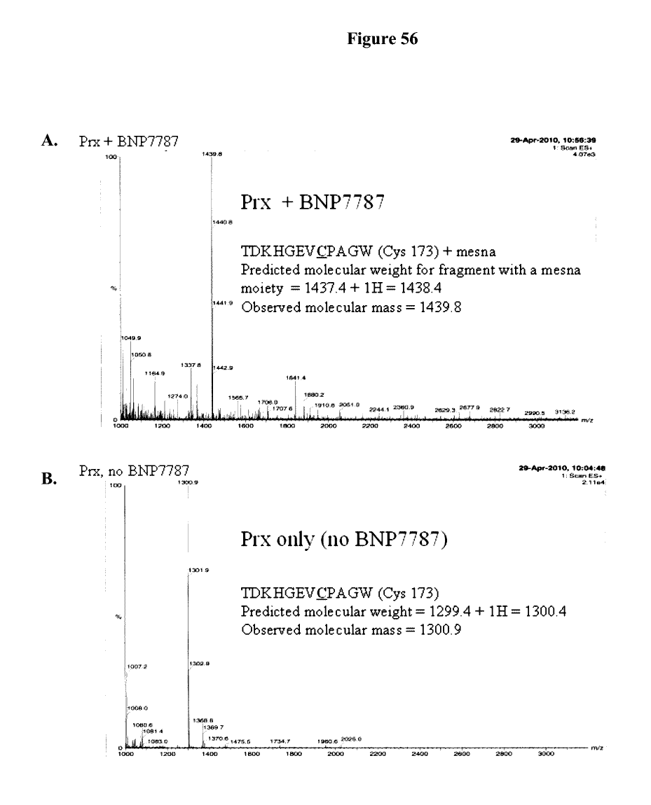

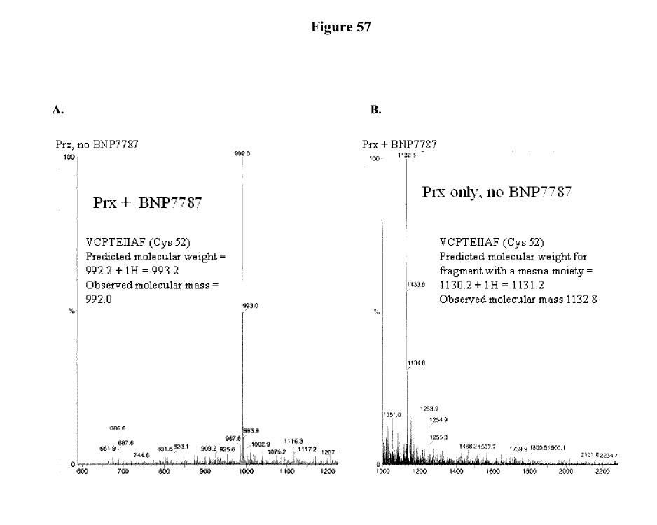

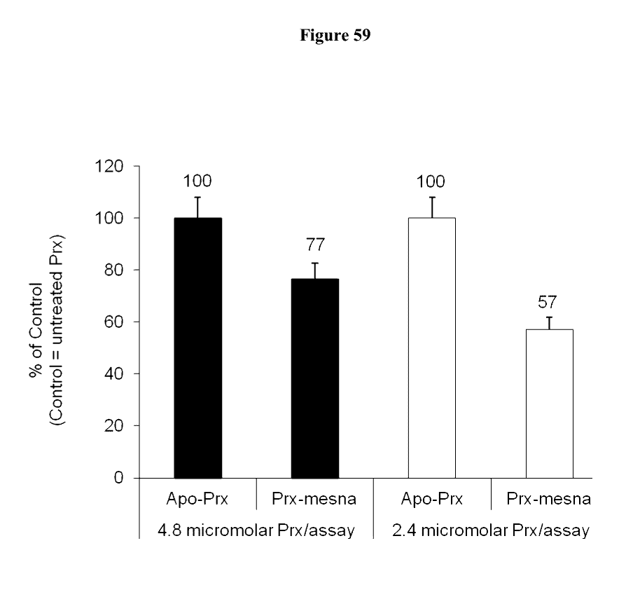

The following experiments were designed to determine if Tavocept forms a detectable, covalent modification on Prx1 and/or Prx 4. Specifically, these studies address whether Tavocept can undergo thiol-disulfide exchange with selected cysteine residues on Prx resulting in formation of a Tavocept-derived mesna-cysteine mixed disulfide. LC-MS studies indicate that a Tavocept-derived mesna-cysteine mixed disulfide forms on Prx 1 on cysteine residues 173, 52, and 71. See, FIG. 55, FIG. 56, and FIG. 57. The results of X-ray crystallographic experiments disclosed in the following sections unequivocally confirm that Tavocept forms a mixed-disulfide with cysteine (Cys) residue 124 of human Prx 4 (see, FIG. 59, infra). Additionally, Mass Spectroscopy data suggests that a second Tavocept-derived mesna moiety modifies Cys 245 on Prx 4, although this region of the crystal structure lacked electron density.

Recombinant human peroxiredoxin 1 (purchased from SigmaAldrich; 333 μg; 0.015 micromoles) was reduced using an excess of dithiothreitol (DTT; 35 micromoles) in NH₄HCO₃ buffer (40 mM, pH 8.0) at 37° C. for 50 minutes (total reaction volume was 750 μL; final Prx concentration was 20 μM; and final DTT concentration was 46 mM). DTT was removed using a G25 Sephadex column (GE Life Sciences) and the DTT-free, reduced protein was incubated with Tavocept (10 mM) or buffer alone at 37° C. (total reaction volume was 500 μL). After 16-18 hours incubation, each reaction was removed and chromatographed using G25 Sephadex columns. This step removed any unreacted Tavocept and was used for the buffer control simply to ensure that both samples received the same handling/manipulation during the course of the experiment (final eluted volume was 1 mL).

In brief, the G25 chromatographed Prx1 incubation reactions were digested with Trypsin Gold. Trypsin Gold was dissolved in 400 μL of NH₄HCO₃ and 100 μL of acetonitrile. A 100-150 μL volume of this Trypsin Gold stock was then added to 500 μl of the aforementioned final sample with 75 μL of acetonitrile and then the reaction volume was adjusted to a total of 750 μL with NH₄HCO₃. The sample was incubated for 6 hours at 37° C. Chymotrypsin digests were by dissolving chymotrypsin in 1M HCL. A 50 μL volume of this chymotrypsin stock solution was added to added to 500 μl of the aforementioned final sample along with 10% (v/v) of CaCl₂ (100 mM stock). NH₄HCO₃ buffer was then added to bring the total reaction volume to 750 μL. Chymotrypsin digests were incubated for 16 hours at 30° C. Following digest by either trypsin or chymotrypsin, reactions were lyophilized to dryness overnight and then resuspended in a minimal amount of HPLC grade water prior to LC MS analyses.

A Symmetry C18 HPLC column (Waters, Franklin, Mass.; 3.5 μm; 4.6×75 mm) and a Waters Alliance liquid chromatography system (Waters 2695; Franklin, Mass.) coupled to a Micromass single quadropole mass detector (Micromass ZMD; Manchester, UK) were used to analyze fragments from trypsin or chymotrypsin digested human Prx1. The mobile phase contained 0.1% of formic acid throughout the run and the flow rate was 0.35 ml/min. The elution scheme involved the following steps: Step 1—0 to 3.5 minutes mobile phase was 95% water/5% acetonitrile; Step 2—3.5 to 20 minutes linear gradient to 10% water/90% acetonitrile; Step 3—20-30 minutes hold at 10% water/90% acetonitrile; Step 4—30-40 minutes linear gradient from 10% water/90% acetonitrile to 95% water; 5% acetonitrile. Positive-ion and negative-ion ionization modes across the mass ranges of 200-1200 Da (positive-ion mode) and 1000-3200 Da (negative-ion mode) were used, respectively.

A. Results of Trypsin Digests on Prx Reactions

Mass spectroscopy analyses revealed the presence of a Tavocept-derived mesna adduct on cysteine-173 of peroxiredoxin. See, Table 17, Row 20: HGEVCPAGWK peroxiredoxin fragment. See also, FIG. 55.

TABLE 17
Tryptic Fragments of Human Prx 1

| Postition | | | | Mass | Mass | Tavocept- |
|---|---|---|---|---|---|---|
| of | | Peptide | Peptide | + | + | derived mesna |
| cleavage | Resulting | length | mass | Mesna | Mesna/Na | Adduct |
| site | peptide sequence | [aa] | [Da] | (add 138) | (add 161) | detected? |
| 7 | MSSGNAK | 7 | 693.8 | | | |
| 16 | IGHPAPNFK | 9 | 980.1 | | | |
| 27 | ATAVMPDGQFK | 11 | 1164.3 | | | |
| 35 | DISLSDYK | 8 | 940.0 | | | |
| 37 | GK | 2 | 203.2 | | | |
| 62 | YVVFFFYPLDFTFVCPTEII | 25 | 3037.5 | 3198.5 | | |
| | AFSDR (Cys 52) | | | | | |
| 67 | AEEFK | 5 | 622.7 | | | |
| 68 | K | 1 | 146.2 | | | |
| 92 | LNCQVIGASVDSHFCHLA | 24 | 2640.0 | 2801.3 | 2801.3 | |
| | WVNTPK (Cys 71,Cys 83) | | | (1 adduct) | (1 adduct) | |
| | | | | 2964.3 | 2964.3 | |
| | | | | (2 adducts) | (2 adducts) | |
| 93 | K | 1 | 146.2 | | | |
| 109 | QGGLGPMNIPLVSDPK | 16 | 1622.9 | | | |
| 110 | R | 1 | 174.2 | | | |
| 120 | TIAQDYGVLK | 10 | 1107.3 | | | |
| 128 | ADEGISFR | 8 | 894.0 | | | |
| 136 | GLFIIDDK | 8 | 920.1 | | | |
| 140 | GILR | 4 | 457.6 | | | |
| 151 | QITVNDLPVGR | 11 | 1211.4 | | | |
| 158 | SVDETLR | 7 | 818.9 | | | |
| 168 | LVQAFQFTDK | 10 | 1196.4 | | | |
| 178 | HGEVCPAGWK (Cys 173) | 10 | 1083.2 | 1221.2 | 1244.2 | Yes |
| 190 | PGSDTIKPDVQK | 12 | 1284.4 | | | |
| 192 | SK | 2 | 233.3 | | | |
| 197 | EYFSK | 5 | 672.7 | | | |
| 199 | QK | 2 | 274.3 | | | |

B. Results of Chymotrypsin Digests on Prx Reactions Chymotrypsin reactions also detected a Tavocept-derived mesna moiety on cys-173 of Prx (see. FIG. 56). See, also, Table 18, Row 19; TDKHGEVCPAGW, as well as cys-52 of Prx (FIG. 57); Table 11, Row 10: VCPTEIIAF.

TABLE 18
Chymotryptic Fragments of Human Prx 1

| Postition | | | | Mass | Mass | Tavocept- |
|---|---|---|---|---|---|---|
| of | | Peptide | Peptide | + | + | derived mesna |
| cleavage | Resulting | length | mass | Mesna | Mesna/Na | Adduct |
| site | peptide sequence | [aa] | [Da] | (add 138) | (add 161) | detected? |
| 15 | MSSGNAKIGHPAPNF | 15 | 1527.7 | | | |
| 26 | KATAVMPDGQF | 11 | 1164.3 | | | |
| 34 | KDISLSDY | 8 | 940.0 | | | |
| 38 | KGKY | 4 | 494.6 | | | |
| 41 | VVF | 3 | 363.5 | | | |
| 42 | F | 1 | 165.2 | | | |
| 43 | F | 1 | 165.2 | | | |
| 48 | YPLDF | 5 | 653.7 | | | |
| 50 | TF | 2 | 266.3 | | | |
| 59 | VCPTEIIAF (Cys 52) | 9 | 992.2 | 1130.2 | 1153.2 | Yes |
| 66 | SDRAEEF | 7 | 852.8 | | | |
| | Or | | Or | | | |
| | SDRAEEFKKL | | 1222.3 | | | |
| 82 | KKLNCQVIGASVDSHF (Cys 71) | 16 | 1746.0 | 1884.0 | 1907.0 | |
| | Or | | Or | Or | Or | |
| | NCQVIGASVDSHF (Cys 71) | | 1376.5 | 1514.5 | 1537.5 | |
| 87 | CHLAW (Cys 83) | 5 | 628.7 | 766.7 | 789.7 | |
| 116 | VNTPKKQGGLGPMNIPLVSDP | 29 | 3138.6 | | | |
| | KRTIAQDY | | | | | |
| 127 | GVLKADEGISF | 11 | 1135.3 | | | |
| 131 | RGLF | 4 | 491.6 | | | |
| 163 | IIDDKGILRQITVNDLPVGRSVD | 32 | 3595.2 | | | |
| | ETLRLVQAF | | | | | |
| 165 | QF | 2 | 293.3 | | | |
| 177 | TDKHGEVCPAGW (Cys 173) | 12 | 1299.4 | 1437.4 | 1460.4 | Yes |
| 194 | KPGSDTIKPDVQKSKEY | 17 | 1920.2 | | | |
| 195 | F | 1 | 165.2 | | | |
| 199 | SKQK | 4 | 489.6 | | | |

II. Effect of Tavocept on PRX 1 Activity

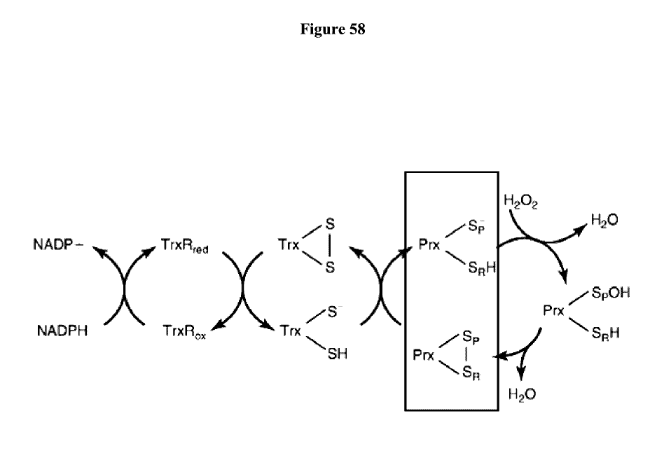

The effect of Tavocept on Prx 1 activity was determined using a Prx assay that was coupled to thioredoxin (Trx), thioredoxin reductase (TrxR), and NADPH. See, FIG. 58. The Applicants of the present patent application have previously disclosed that Tavocept is an alternative substrate inhibitor of Trx. Therefore, to prevent interference from Trx-mediated reduction of Tavocept, all unreacted/free Tavocept must be removed prior to assaying Prx.

In brief, recombinant human Prx 1 (250 μg; 0.011 micromoles) was reduced using an excess of dithiothreitol (DTT; 83 mM final concentration) in NH₄HCO₃ buffer (40 mM, pH 8.0) at 37° C. for 1 hour (total volume was 250 μM. DTT was removed using a Nap5 G25 Sephadex column (GE Life Sciences) and the DTT-free, reduced protein was incubated with Tavocept (20 mM) or buffer alone at 37° C. (final reaction volume was 370 μL) for 16 hours. The Tavocept and buffer only incubation reactions were removed and chromatographed over G25 Sephadex columns. This step removed unreacted Tavocept and was used for the buffer control simply to ensure that both samples received the same handling/manipulation during the course of the experiment (final eluted volume was 850 μL).

Prx activity in the buffer control (Apo-Prx) and the Tavocept-treated sample (Prx-mesna) was determined using the Prx assay outlined in FIG. 58. A typical assay mixture was 150 μL in final volume and contained HEPES Buffer (45 mM, pH 7.0, 1 mM EDTA), 350 μM NADPH, 0.12 μM rat liver TrxR, 80 μM insulin, 2.9 μM human Trx 1, and Prx (4.8 or 2.4 μM per assay). Assays were initiated by the addition of H₂O₂ (100 μM) and were monitored at 340 nm at 25° C. for 30 minutes using a using a Molecular Devices SpectraMax Plus UV/vis plate reader. Activity was calculated using a 4 minute linear portion of each assay and then this rate was converted into percent of control with the buffer treated Prx 1 reaction (Apo-Prx) serving as a 100% control. See, FIG. 59. When Prx 1 is incubated with Tavocept the rate is notably reduced relative to a control where Prx 1 is incubated only with buffer (apo-Prx). This reduction in rate is approximately 43% in assays containing 2.4 μM Prx and 23% in assays containing 4.8 μM Prx. See, FIG. 59. The more pronounced effect at the lower Prx concentration of 2.4 μM is likely due to the fact that as the concentration of Prx is increased, there is a higher percentage of unmodified Prx present in the assay in the Prx 1 samples incubated with Tavocept. Therefore, as the concentration of unmodified Prx increases, the turnover more nearly approaches the buffer control Apo-Prx samples.

III. Specific Example and Experimental Results for Peroxiredoxin 4

Wild-Type human peroxiredoxin 4 (PRX4) was cloned into a proprietary vector containing an N-terminal 6×his tag cleavable by TEV protease using the following primers: 5′-TATATA GGT ACC GCG AAG ATT TCC AAG CC-3′ and 5′-TATATA CTC GAG TCA ATT CAG TTT ATC GAA AT-3′. Final product was sequence verified. The final product was expressed in BL21(RIPL) cells. Cells containing the human PRX4 construct were grown at 37° C. to OD₆₀₀˜0.6. The cells were induced with 0.5 mM IPTG at 18° C. overnight. Cell biomass was harvested and stored at −80° C. until ready to use. Purification of target protein was done in a 2 column system. The cell biomass was lysed by sonification in 50 mM Tris-HCl pH 7.8, 500 mM NaCl, 10% glycerol, 20 mM imidazole, 5 mM BME (Buffer A) plus 1 Roche Complete Protease Inhibitor Tablet, and 20,000 units Benzonase. Target protein was extracted by binding to Ni2+ charged IMAC resin and eluted using a gradient of 0-500 mM Imidazole (the N-terminal tag was not cleaved). Peak fractions were pooled and aggregation was separated from monomeric protein via Size Exclusion in 10 mM HEPES pH 8.5, 300 mM NaCl, 5% glycerol, and 5 mM DTT. Monomeric protein was concentrated to ˜12.6 mg/mL.

The adduct was prepared by incubating human Prx 4 (12.6 mg/mL) in 10 mM HEPES pH 8.5, 300 mM NaCl, 5% glycerol, and 60 mM DTT at 30° C. for 1 hour and then overnight at 4° C. Excess DTT was removed by dialyzing in 10 mM HEPES pH 8.5, 300 mM NaCl, and 5% glycerol. The Prx protein (12 mg/mL) was supplemented with 1 mM DTPA, 1 mM Neocuprione, and 40 mM Tavocept and incubated at 4° C. overnight. The protein was then characterized by Mass Spectrometry. The Mass Spectroscopy data suggested that protein going into crystallization had two (2) to three (3) Tavocept-derived mesna metabolite adducts per Prx molecule.

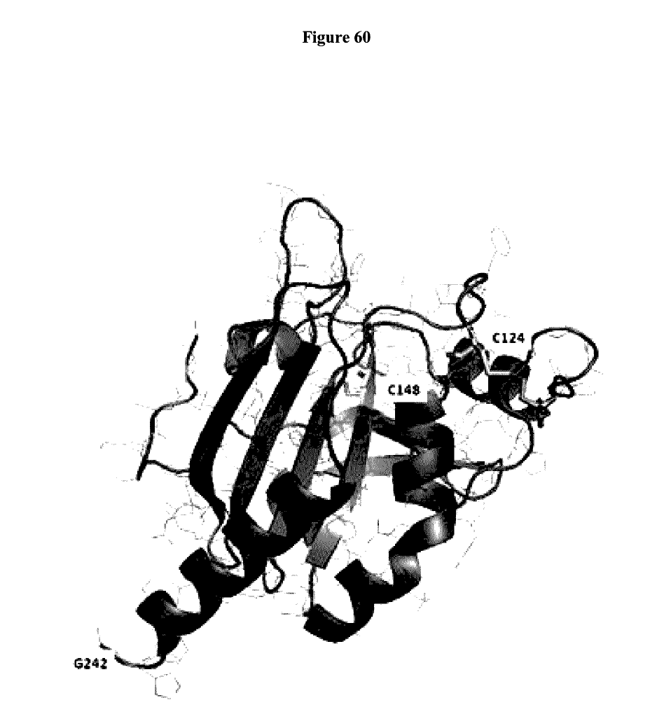

Co-crystals of human Prx 4 with Tavocept-derived mesna moieties appeared in 2-3 conditions with the best crystals growing from 1.6 M ammonium sulfate, 0.1 M MES pH 6.5, 10% dioxane and also in 0.2 M ammonium phosphate. See, FIG. 60. Before data collection, the crystals were transferred into a cryoprotectant solution made up of 35% glycerol v/v in crystallization buffer, after which they were flash-frozen in liquid nitrogen for data collection. Crystals of the ammonium sulfate condition diffracted to 2.3 Å containing five molecules in the asymmetric unit (½ of the decamer donut biological unit).

Lower resolution ammonium phosphate crystals were transferred into a cryoprotectant solution made up of 40% glycerol (v/v) in crystallization buffer, after which they were flash-frozen in liquid nitrogen for data collection. These crystals diffracted to 2.95 Å. The crystal is space group P21212 with 10 molecules (one decamer donut biological unit) in the asymmetric unit. This lower resolution structure agrees with the conformational changes observed in the high resolution structure adduct structure and also strongly suggested one Tavocept-derived mesna moiety bound at Cys124.

Diffraction data for the 2.3 Å structure ammonium sulfate crystals (1.6M ammonium sulfate, 0.1 M MES pH 6.5, 10% dioxane) were collected at a wavelength of 1.0 Å on a Rayonix 300 detector array at beamline CLS-08ID at the Canadian Light Source, Saskatchewan, Canada. Imaging processing statistics are shown in Table 19.

TABLE 19
Final Statistics for Data Evaluation
(statistics for final resolution shell are shown in parentheses)

| | |
|---|---|
| Unit cell (Å) | 108.004 139.684 96.191 90.000 103.172 90.000 |
| Space group | C2 |
| Resolution range (Å) | 50.00-2.27 (2.35-2.27) |
| No. of observations | 229144 |
| No. of unique | 62860 |
| reflections | |
| Redundancy | 3.6 (3.0) |
| Completeness (%) | 97.8 (92.1) |
| Mean I/sigma(I) | 13.0 (1.8) |
| Rmerge | 0.075 (0.497) |

B. Structure Solution and Refinement

Data were indexed, integrated, scaled and merged using the programs HKL2000 or Mosflm. The structure was solved by molecular replacement with PHASER using a monomer from the Protein Data Bank entry for human PRX4 (PDBID 2PN8) as the search model. The solution was consistent with five molecules in the crystal asymmetric unit. The protein model was iteratively refit and refined using MIFit (see, MIFit Open Source Project, 2010 at http://code.google.com/p/mifit) and REFMAC5 (see, Murshudov, et al., Refinement of macromolecular structures by the maximum-likelihood method. Acta Crystallogr. D. Biol. Crystallogr. 1:53(Pt 3):240-255 (1997). The segment from residues 121 to 126 containing Cys124 is in a significantly altered conformation compared to the apo structure (PDBID 2PN8) and is consistent with the presence of a Tavocept-derived mesna adduct. The Tavocept-derived mesna moiety appears slightly disordered in the electron density map most likely due to its location on the surface of the protein. It is modeled in multiple conformations for some of the PRX4 monomers (chains B, D, E). There is no density present beyond residue 242. The last 27 residues of the C-terminus of each monomer are thus not included in the final refined structure (see, Table 20).

TABLE 20
Crystallographic Data and Refinement Statistics

| | |
|---|---|
| Resolution range (Å) | 33.057-2.256 |
| No. of reflections | 62543 (59375 working set, 3168 |
| | test set) |
| No. of protein chains | 5 (A, B, C, D, E) |
| Ligand id code | UNK |
| No. of protein residues | 830 |
| No. of ligands | 5 |
| No. of waters | 191 |
| No. of atoms | 6836 |
| Mean B-factor | 51.502 |
| Rwork | 0.2084 |
| Rfree | 0.2494 |
| Rmsd bond lengths (Å) | 0.013 |
| Rmsd bond angles (°) | 1.367 |
| Number of disallowed φψ angles | 0 |

C. Protein Assembly and Domain Structure

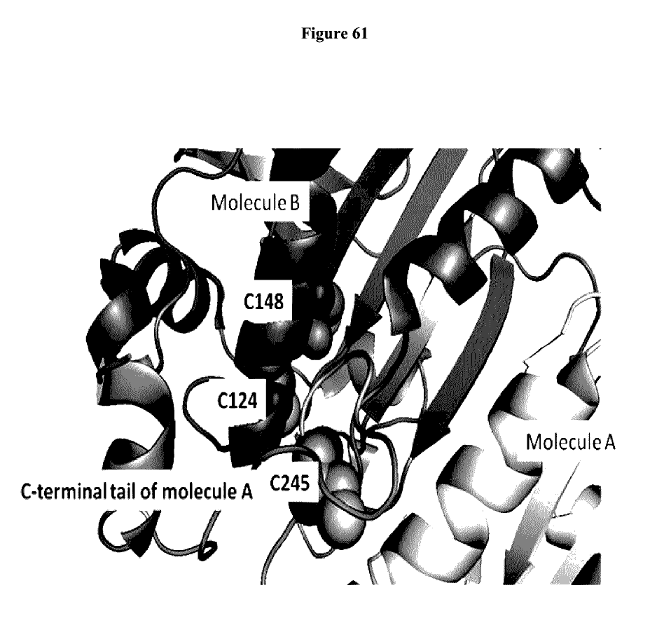

Prx4 forms a donut shaped decamer. In the native apo structure (PDBID 2PN8), a C-terminal tail (starting at Gly242) wraps around a neighboring molecule forming an extended interface. At this interface, Cys124 from one molecule is in close proximity to Cys245 located on the C-terminal tail of an adjacent molecule (see, FIG. 61). Cys124 and Cys245 are catalytically important, conserved, active site residues.

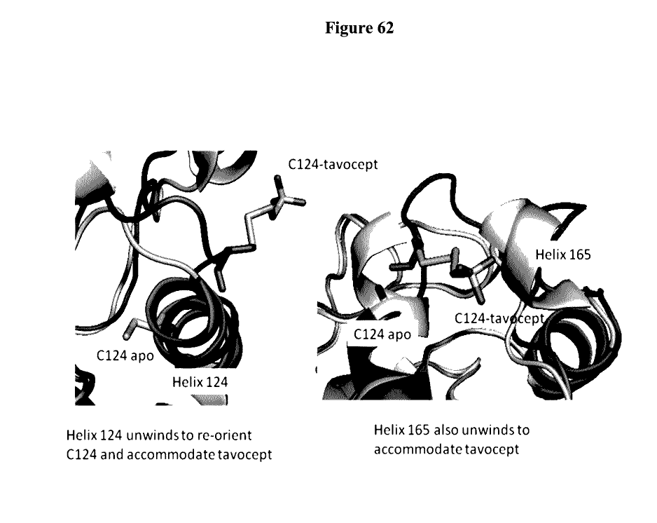

The Tavocept-derived mesna moiety was found to bind to Cys124 in all 5 molecules. In some molecules, the Tavocept-derived mesna moiety is bound in multiple conformations. To accommodate the Tavocept-derived mesna moiety, it appears that residues 121-126 undergo a conformational change, partially unwinding the helix and exposing Cys124 (see, FIG. 62). C148 is buried, well-ordered and does not appear to interact with the Tavocept-derived mesna moiety. The structure beyond residue 242, containing C245 is disordered. It should be noted that the presence of a Tavocept-derived mesna at position C124 would interfere with the placement of the section of structure containing F267 from a neighboring molecule in the assembly.

D. Second Tavocept-Derived Mixed Disulfide on Prx

The absence of density beyond residue 242 is most likely due to a lack of a defined secondary or tertiary structure for this sequence. As the binding of a Tavocept-derived mesna moiety would sterically interfere with the docking of the C-terminus at the binding site observed in the native apo structure, it is very likely that this segment is no longer composed of defined structural elements which would permit visualization in an electron density map. Specifically, Tyr 266 and Phe267 of the native structure form a hydrophobic patch on the C-terminal helix and occupy the space occupied by the tavocept-derived mesna moiety and Cys124. Disruption of this interaction could destabilize this helix further contributing to a lack of structure.

The Mass Spectroscopy analyses of the crystals were consistent with intact protein. Therefore, it is unlikely that the lack of density found was due to proteolysis of the tail. The Mass Spectroscopy analyses were also consistent with the presence of two Tavocept-derived mesna moieties suggesting that Cys245 may have a Tavocept-derived mesna adduct bound. Tavocept binding at Cys245 would be expected to further interfere with the docking of the C-terminal tail. Because there are no data (electron density) for the C-terminal tail, it was not able to be modeled and one may assume it is in multiple perhaps unstructured conformations.

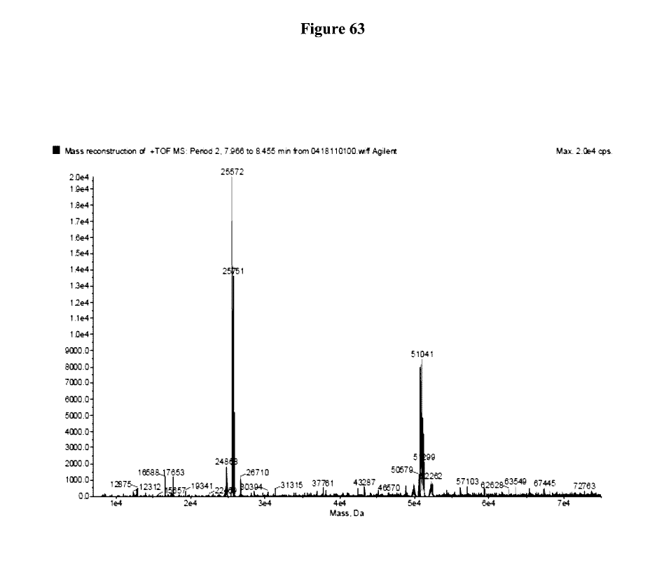

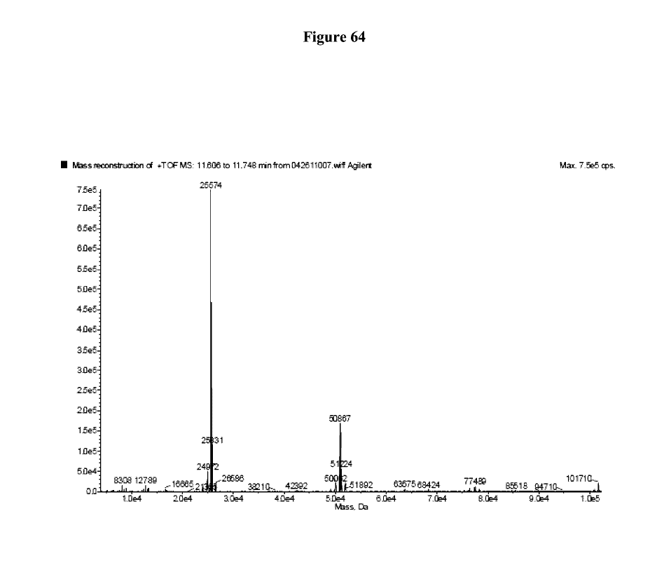

The Mass Spectroscopy data (see, FIG. 63) suggested that the Prx 4 protein contained two (2) Tavocept-derived mesna moieties after reaction with Tavocept and the Mass Spectroscopy analysis of the dissolved crystals (see, FIG. 64) show that the monomer has two (2) Tavocept-derived mesna adducts. The mass of native protein is 25292, which is consistent with the N-terminal Met being removed.

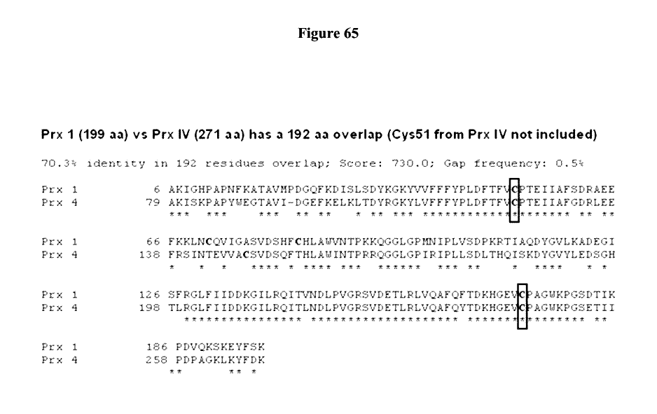

In summary, Tavocept has been shown to modify human Prx 1 on cysteines 52 and 173 by LC and MS analysis and human Prx 4 on cysteine 124 (and possibly cysteine 245) by MS and X-ray crystallographic analyses. Cysteine 52 of Prx 1 corresponds to cysteine 124 of Prx 4 and cysteine 173 of Prx 1 corresponds to cysteine 245 of Prx 4 (see, FIG. 65). Cysteine 52 and cysteine 173 in Prx 1 are the catalytically important cysteine residues and, likewise, cysteine 124 and cysteine 245 in Prx 4 are the catalytically important cysteine residues. Therefore, the Tavocept-derived mesna mixed disulfide that forms on these cysteine residues is of key functional significance. Additionally both Prx 1 and Prx 4 are highly expressed in NSCLC and Lehtonen, et al. have reported that Prx 4 may be preferentially expressed in the adenocarcinoma sub-type of non-small cell lung cancer (NSCLC). See, Lehtonen, et al., Prx a novel family in lung cancer. Int. J. Cancer 111:514-521 (2004). Additionally, Prx isoforms have been shown to be overexpressed in endometrial, breast, colorectal, and prostate cancers. See, e.g., Seulhee, Han, Haiying, Shen, et al., Expression and prognostic significance of human peroxiredoxin isoforms in endometrial cancer. Oncology Letters 3:(6) 1275-1279 (2012).

(ii) The Thioredoxin Reductase/Thioredoxin System

The thioredoxin system is comprised of thioredoxin reductase (TrxR) and its main protein substrate, thioredoxin (Trx), where the catalytic site disulfide of Trx is reduced to a dithiol by TrxR at the expense of NADPH. The thioredoxin system, together with the glutathione system (comprising NADPH, the flavoprotein glutathione reductase, glutathione, and glutaredoxin), is regarded as a main regulator of the intracellular redox environment, exercising control of the cellular redox state and antioxidant defense, as well as governing the redox regulation of several cellular processes. The system is involved in direct regulation of: (i) several transcription factors; (ii) apoptosis (i.e., programmed cell death) induction; and (iii) many metabolic pathways (e.g., DNA synthesis, glucose metabolism, selenium metabolism, and vitamin C recycling).

Thioredoxin reductases are homodimers present in the cytosol, nucleus (TrxR-1), and mitochondria (TrxR-2). Thioredoxins also are present both in the cytosol (Trx-1) and mitochondria (Trx-2), and the cytosolic isoform can also enter the nucleus. The thioredoxin system has a crucial role in regulating functions such as cell viability and proliferation via a thiol redox state. See, e.g., Lillig, C. H. Holmgren, A. Thioredoxin and related molecules—from biology to health and disease. Antioxid. Redox Signal. 9:25-47 (2007). Thioredoxins act as electron donors for a number of enzymes, such as ribonucleotide reductase, methionine sulfoxide reductase, and peroxiredoxins. See, e.g., Levine, R. L., Moskovitz, J., Stadtman, E. R. Oxidation of methionine in proteins: roles in antioxidant defense and cellular regulation. IUBMB Life. 50:301-307 (2000). The latter may be active as antioxidants by rapidly regulating the level of hydrogen peroxide (see, e.g., Rhee, S. G., Chae, H. Y., Kim, K. Peroxiredoxins: a historical overview and speculative preview of novel mechanisms and emerging concepts in cell signaling. Free Radical Biol Med. 38:543-1552 (2005)). but, depending on the conditions, may also influence the redox state of thioredoxins that can exert a central role in the redox regulation of signaling molecules and transcription factors. This role, mediating the cellular response to changes in the redox state, is further complemented by glutaredoxins. See, e.g., Lillig, C. H., Holmgren, A. Thioredoxin and related molecules—from biology to health and disease. Antioxid. Redox Signal. 9:25-47 (2007). Thioredoxin-1 (Trx-1), in its reduced form, binds to ASK1 and inhibits its activity, acting therefore as a negative effector of apoptosis. However, because this inhibition is removed after oxidation of thioredoxin, which dissociates from ASK1 (see, e.g., Saitoh, M., Nishitoh, H., et al. Mammalian thioredoxin is a direct inhibitor of apoptosis signal-regulating kinase (ASK) 1. EMBO J. 17:2596-2606 (1998)), it is clearly apparent that thioredoxin acts as a redox sensor of ASK1. Recently, endogenous generation of H₂O₂ by stimulation of Nox2 in alveolar macrophages was shown to activate ASK1 through the oxidation of thioredoxin-1. See, e.g., Liu, H., Zhang, H., et al. The ADP-stimulated NADPH oxidase activates the ASK-1/MKK4/JNK pathway in alveolar macrophages. Free Radical Res. 40:865-874 (2006).

Several transcription factors depend on redox-sensitive cysteines, and their function is modulated by the redox state of thioredoxin, which, in turn, reflects the cellular redox state. The activity of the transcription factor NF-κB is inhibited in the cytosol by reduced thioredoxin. In contrast, reduced thioredoxin activates this transcription factor in the nucleus by promoting its binding to DNA. See, e.g., Kabe, Y., Ando, K., et al. Redox regulation of NF-κB activation: distinct redox regulation between the cytoplasm and the nucleus. Antioxid. Redox Signal. 7:395-403 (2005). Other transcription factors, sensitive to thioredoxin, are the tumor-suppressor p53 (see, e.g., Ueno, M., Masutani, H., et al. Thioredoxin-dependent redox regulation of p53-mediated p21 activation. J. Biol. Chem. 274:35809-35815 (1999)), the hypoxia-inducible factor 1α (HIF-1α; see, e.g., Welsh, S. J., Bellamy, W. T., et al. The redox protein thioredoxin-1 (Trx-1) increases hypoxia-inducible factor 1 alpha protein expression: Trx-1 overexpression results in increased vascular endothelial growth factor production and enhanced tumor angiogenesis. Cancer Res. 62:5089-5095 (2003)), the glucocorticoid receptor (see, e.g., Makino, Y., Yoshikawa, N., et al. Direct association with thioredoxin allows redox regulation of glucocorticoid receptor function. J. Biol. Chem. 274:3182-3188 (1999)), and the AP-1 protein complex (see, e.g., Hirota, K., Matsui, M., et al. AP-1 transcriptional activity is regulated by a direct association between thioredoxin and Ref-1. Proc. Natl. Acad. Sci. USA 94:3633-3638 (1997)). The latter is activated by the direct association of Trx with redox factor-1 (Ref-1). Redox factor-1 is a nuclear 37-kDa enzyme that, in addition to a DNA-repair function, possess two redox-sensitive cysteines at positions 63 and 95. Ref-1, by reducing critical cysteines, also facilitates the binding to DNA of several transcription factors, including NF-κB, p53, and HIF-1α. Ref-1 deficiency renders cells more sensitive to apoptosis, as shown by its knockdown by small interfering RNA (siRNA). See, e.g., Yang, S., Misner, B. J., et al. Redox effector factor-1, combined with reactive oxygen species, plays an important role in the transformation of JB6 cells. Carcinogenesis 28:2382-2390 (2007). Thioredoxin strictly cooperates with Ref-1 as phorbol esters treatment of COS-7 cells stimulates the translocation to the nucleus of thioredoxin, which, in turn, potentiates AP-1 activity.

The Nrf2-Keap1 system is recognized as a major cell-defense mechanism against oxidative stress and xenobiotics and plays a key role in upregulating phase 2 enzymes. In cytoplasm, the transcription factor Nrf2 is associated with a specific repressor protein, Keap1, that inhibits its translocation to the nucleus, but also acts as a participant in causing the rapid turnover of Nrf2 by ubiquitination and degradation. Keap-1 is a redox-sensitive protein with several cysteines. Some of them (Cys²⁷³ and Cys²⁸⁸) act as “reactive cysteines” and, on interaction with ROS or electrophiles, undergo oxidation or covalent modification, thereby facilitating the dissociation of the Nrf2-Keap1. Consequently, Nrf2 can translocate to the nucleus, where it accelerates the transcription of phase 2 genes, including thioredoxin and thioredoxin reductase genes. See, e.g., Kim, Y. C., Yamaguchi, Y., et al., Thioredoxin-dependent redox regulation of the antioxident response element (ARE) in electrophile response. Oncogene 22:1860-1865 (2003). The role of Trx in cell growth and development, its antioxidant action, and thiol redox regulation of transcription factors provides a rationale for the observed upregulation of thioredoxin in several types of cancers. See, e.g., Berggren, M., Gallegos, A., et al., Thioredoxin and thioredoxin reductase gene expression in human tumors and cell lines, and the effects of serum stimulation and hypoxia. Anticancer Res. 16:3459-3466 (1996). Association of this upregulation with resistance to apoptosis makes Trx and TrxR relevant targets for anti-tumor therapy.

A. Thioredoxin Reductase (TrxR)

The mammalian thioredoxin reductases (TrxRs) are enzymes belonging to the avoprotein family of pyridine nucleotide-disulfide oxidoreductases that includes lipoamide dehydrogenase, glutathione reductase, and mercuric ion reductase. Members of this family are homodimeric proteins in which each monomer includes an FAD prosthetic group, an NADPH binding site and an active site containing a redox-active disulfide. Electrons are transferred from NADPH via FAD to the active-site disulfide of Trx, which then reduces the substrate. See, e.g., Williams, C. H., Chemistry and Biochemistry of Flavoenzymes (Muller, F., ed.), pp. 121-211, CRC Press, Boca Raton (1995).

TrxRs are named for their ability to reduce oxidized thioredoxins (Trxs), a group of small (i.e., 10-12 kDal), ubiquitous redox-active peptides that undergoes reversible oxidation/reduction of two conserved cysteine (Cys) residues within the catalytic site. The mammalian TrxRs are selenium-containing flavoproteins that possess: (i) a conserved -Cys-Val-Asn-Val-Gly-Cys-catalytic site; (ii) an NADPH binding site; and (iii) a C-terminal Cys-Selenocysteine sequence that communicates with the catalytic site and is essential for its redox activity. See, e.g., Powis, G. Monofort, W. R. Properties and biological activities of thioredoxins. Ann. Rev. Pharmacol. Toxicol. 41:261-295 (2001). These proteins exist as homodimers and undergo reversible oxidation/reduction. The activity of TrxR is regulated by NADPH, which in turn is produced by glucose-6-phosphate dehydrogenase (G6DP), the rate-limiting enzyme of the oxidative hexose monophosphate shunt (HMPS; also known as the pentose phosphate pathway). Two human TrxR isozyme genes have been cloned: (i) the gene for human TrxR-1 located on chromosome 12q23-q24.1 encoding a 54 Kda enzyme that is found predominantly in the cytoplasm; and (ii) the gene for human TrxR-2 located on chromosome 22q11.2 encoding a 56 Kda enzyme the possesses a 33-amino-acid N-terminal extension identified as a mitochondrial import sequence. See, e.g., Powis, G. Monofort, W. R. Properties and biological activities of thioredoxins. Ann. Rev. Pharmacol. Toxicol. 41:261-295 (2001). A third isoform of TrxR, designated (TGR) is a Trx and glutathione reductase localized mainly in the testis, has also been identified. See, e.g., Sun, Q. A., et al. Selenoprotein oxidoreductase with specificity for thioredoxin and glutathione systems. Proc. Natl. Acad. Sci. USA 98:3673-3678 (2001). Additionally, both mammalian cytosolic TrxR-1 and mitochondrial TrxR-2 have alternative splice variants. In humans, five different 5′ cDNA variants have been reported, with one of the splice variants comprising a 67 kDa protein with an N-terminal elongation, instead of the common 55 kDa. The physiological functions of these TrxR splice variants have yet to be elucidated. See, e.g., Sun, Q. A., et al. Heterogeneity within mammalian thioredoxin reductases: evidence for alternative exon splicing. J. Biol. Chem. 276:3106-3114 (2001).

The TrxR-1 isozyme has been the most extensively studied. TrxR-1, as purified from tissues such as placenta, liver, or thymus, and expressed in recombinant form, possesses wide substrate specificity and generally high reactivity with electrophilic agents. The catalytic site of TrxR-1 encompasses an easily accessible selenocysteine (Sec) residue situated within a C-terminal motif comprising -Gly-Cys-Sec-Gly-COOH. See, e.g., Zhong, L., et al. Rat and calf thioredoxin reductase are homologous to glutathione reductase with a carboxyl-terminal elongation containing a conserved catalytically active penultimate selenocysteine residue. J. Biol. Chem. 273:8581-8591 (1998). Together with the neighboring cysteine, it forms a redox-active selenenylsulfide/selenolthiol motif that receives electrons from a redox-active -Cys-Val-Asn-Val-Gly-Cys-motif present in the N-terminal domain of the other subunit in the dimeric enzyme. See, e.g., Sandalova, T., et al. Three-dimensional structure of a mammalian thioredoxin reductase: implications for mechanism and evolution of a selenocysteine-dependent enzyme. Proc. Natl. Acad. Sci. USA 98:9533-9538 (2001). Substrates of the TrxR-1 enzyme, that can be reduced by the selenolthiol motif, include: protein disulfides such as those in thioredoxin; NK-lysin; protein disulfide isomerase; calcium-binding proteins-1 and -2; and plasma glutathione peroxidase; as well as small molecules such as 5,5′-dithiobis(2-nitrobenzoate) (DTNB); alloxan; selenodiglutathione; methylseleninate; S-nitrosoglutathione; ebselen; dehydroascorbate; and alkyl hydroperoxides. See, e.g., Amk, E. S., et al. Preparation and assay of mammalian thioredoxin and thioredoxin reductase. Method. Enzymol. 300:226-239 (1999). Additionally, several quinone compounds can be reduced by the enzyme and one-electron reduced species of the quinones may furthermore derivatize the selenolthiol motif, thereby inhibiting the enzyme. The highly accessible selenenylsulfide/selenolthiol motif of the enzyme is extraordinarily reactive and can be rapidly derivatized by various electrophilic compounds.

Due to the many important functions of TrxR, it is not surprising that its inhibition could be deleterious to cells due to an inhibition of the whole thioredoxin system. Moreover, in addition to a general inhibition of the thioredoxin system as a mechanism for cytotoxicity, it has also been shown that selenium-compromised forms of TrxR may directly induce apoptosis in cells by a gain of function. See, e.g., Anestal, K., et al. Rapid induction of cell death by selenium-compromised thioredoxin reductase 1, but not by the fully active enzyme containing selenocysteine. J. Biol. Chem. 278:15966-15672 (2003). The signaling mechanisms of this apoptotic induction have not been presently elucidated. It is clear, however, that electrophilic compounds inhibiting TrxR may have significant cellular toxicity as a result of these effects. From these findings it may surmised that TrxR inhibition may be regarded as a potentially important mechanism by which several alkylating agents and various cancer treating agents (e.g., the monohydrated complex of cisplatin, oxaliplatin, etc.) commonly utilized in anticancer treatment, may exert their cytotoxic effects.

Some of the major functions of mammalian Trx proteins are to supply reducing equivalents to enzymes such as ribonucleotide reductase and thioredoxin peroxidase, as well as (through thiol-disulphide exchange) to reduce key Cys residues in certain transcription factors, resulting in their increased binding to DNA and altered gene transcription. Mammalian Trxs have also been shown to function as cell growth factors and to inhibit apoptosis. Since TrxRs are the only class of enzymes known to reduce oxidized Trx, it is possible that alterations in TrxR activity may regulate some of the activities of Trxs. In addition to Trxs, other endogenous substrates have been demonstrated for TrxRs, including, but not limited to: lipoic acid, lipid hydroperoxides, the cytotoxic peptide NK-lysin, vitamin K₃, dehydroascorbic acid, the ascorbyl free radical, and the tumor-suppressor protein p53. See, e.g., Mustacich, D., Powis, G. Thyrodoxin Reductase. Biochem. J. 346:1-8 (2000). However, the physiological role that TrxRs play in the reduction of most of these substrates has not been fully elucidated.

Another thiol redox system found in cells is the glutathione reductase/glutathione system which, like the TrxR/Trx system, utilizes NADPH as its source of reducing equivalents. There is no known functional interaction between the two systems. The glutathione system plays a key role in protecting cellular macromolecules from damage due to reactive oxygen species (ROS) and electrophilic species. See, e.g., Reed, D. J. (1995) Molecular and Cellular Mechanisms of Toxicity (DeMatteis, F. and Smith, L. L., eds.), pp. 35-68, CRC Press, Boca Raton. Common features of the TrxR and glutathione reductase systems include: (i) an enzyme that is a member of the pyridine nucleotide-disulphide oxidoreductase family; (ii) a small redox-active peptide—Trx and glutaredoxin, respectively; and (iii) the ability to undergo thiol-disulphide exchange. Differences of the TrxR and glutathione reductase systems include: (i) the limited substrate specificity of glutathione reductase, which only reduces glutathione; and (ii) the high intracellular levels of reduced glutathione, which removes electrophiles by both spontaneous and glutathione transferase-catalysed mechanisms.

### TxrR Catalytic Mechanisms

The catalytic mechanism of E. coli TrxR has been extensively studied. See, e.g., Lennon, B. W. Williams, Jr., C. H. Biochemistry 36:9464-9477 (1997). The spatial orientation of the NADPH and FAD domains of E. coli TrxR are such that the nicotinamide ring of NADPH bound to the enzyme does not make close contact with the isoalloxazine ring of FAD, as it does in other members of the pyridine nucleotide-disulphide oxidoreductase family. However, if the NADPH domain of E. coli TrxR is rotated 66° while the FAD domain remains fixed, then the bound NADPH moves into close contact with the isoalloxazine ring; this allows electrons to pass to FAD and then to the active-site disulphide which, when reduced, moves to the surface of the enzyme, where it is accessible to oxidized Trx. See, e.g., Veine, D. M., Ohnishi, K, Williams, Jr., C. H. Protein Sci. 7:369-375 (1998).

In contrast, mammalian TrxRs share a higher degree of sequence identity and mechanistic similarity with glutathione reductase than with E. coli TrxR. In glutathione reductase, the active-site Cys residues, which are in the FAD domain, and the bound NADPH are in close proximity to the isoalloxazine ring of FAD, allowing electrons to flow from NADPH to glutathione via the isoalloxazine ring of FAD and the active-site disulphide without a major conformational change in the enzyme. In the presence of excess NADPH, human TrxR, glutathione reductase, and lipoamide dehydrogenase, but not E. coli TrxR, form a stable thiolate flavin charge-transfer complex, indicative of the mechanistic similarity among these three enzymes. However, titration of human TrxR with dithionite shows the presence of an additional redox-active site that is not present in glutathione reductase. See, e.g., Arscott, L. D., Gromer, S., et al. Proc. Natl. Acad. Sci. U.S.A. 94:3621-3623 (1997). This finding is reminiscent of titration studies with mercuric ion reductase, an oxidoreductase with a second pair of redox-active Cys residues at the C-terminal end of the protein. As discussed above, TrxR1 has a C-terminal SeCys residue that is required for catalytic activity, but is not part of the conserved active site. All other mammalian selenoproteins for which a function is known are redox enzymes with SeCys in the active center.

Mammalian TrxRs are promiscuous enzymes capable of reducing Trxs of different species, proteins such as NK lysin and p53, a variety of physiological substrates (see, e.g., May, J. M., Cobb, C. E., et al. J. Biol. Chem. 273:23039-23045 (1998), as well as several exogenous compounds (see, e.g., Kumar, S., Bjornstedt, M., Holmgren, A. Eur. J. Biochem. 207:435-439 (1992). It may be that it is the C-terminal catalytic SeCys that accounts for the broad substrate specificity of TrxR, allowing the enzyme to reduce bulky proteins as well as small molecules. One suggested catalytic mechanism for human TrxR is that the C-terminal end of the protein is flexible, allowing the -Cys-SeCys-Gly moiety to carry reducing equivalents from the conserved active-site Cys residues to the substrate. See, e.g., Gromer, S., Wissing, J., et al. Biochem. J. 332:591-592 (1998).

### Regulation of TrxR Expression

Both TrxR-1 and TrxR-2 (encoded by the TXNRD1 and TXNRD2 genes) are each also expressed in the form of several isoforms derived from alternative splicing, reflecting a highly complex and cell type-specific expression pattern. See, e.g., Rundlof, A-K., Janard, M., et al. Evidence for intriguingly complex transcription of human thioredoxin reductase 1. Free Rad. Biol. Med. 36:641-656 (2004). Sequences in the 3′ untranslated regions (UTRs) of mRNA confer regulation of expression through a variety of mechanisms, including alterations in mRNA turnover, translation initiation, subcellular localization, and (in the case of selenoenzymes) by dictating the choice between incorporation of SeCys or termination of protein synthesis. One function of the 3′ UTR selenocysteine insertion sequence (SECIS) element is to provide a hierarchy for the expression of selenoproteins under conditions of limited Selenium (Se) availability. Differences in the 3′ UTRs of three selenoproteins, cytoplasmic glutathione peroxidase, phospholipid hydroperoxide glutathione peroxidase, and type 1 deiodinase, result in 2-fold differences in protein expression in response to Se limitation. See, e.g., Bermano, G., Arthur, J. R. Hesketh, J. E. Biochem. J. 320:891-895 (1998). The TrxR-1 SECIS element is highly active under normal conditions, but is less responsive to Se supplementation than the SECIS element of type 1 deiodinase, suggesting that TrxR-1 levels are better maintained when Se supply is low but that protein levels will not increase as dramatically under conditions of Se excess.

The 3′ UTR of TrxR-1 also contains a cluster of six AU-rich elements (AREs), which function to regulate mRNA levels by directing acceleration of the deadenylation process. See, e.g., Xu, N., Chen, C. Y., Shyu, A B. Mol. Cell. Biol. 17:4611-4621 (1997). These mRNA instability elements are typically found in cytokine, growth factor, and proto-oncogene mRNAs that undergo rapid turnover. Inactivation of AREs in growth factor and proto-oncogene mRNAs has been linked to promotion of cellular transformation and oncogenesis. For example, stabilization of c-Myc mRNA due to deletion of AREs promotes oncogenic transformation in vitro and is associated with a human T-cell leukaemia. AREs in the gene encoding TrxR1 may serve to maintain stringent control of TrxR1 expression, thereby preventing the deleterious effects that may be associated with overexpression. It should be noted that the 3′ UTR of TrxR-2 does not contain AREs. See, e.g., Lee, S., Kim, J., et al. J. Biol. Chem. 274:4722-4734 (1999).

### Biological Function of TrxR

The involvement of TrxR in biological functions such as cell growth and protection from oxidative stress has, to date, centred around its role as a reductant for Trx. Further studies are needed to determine whether TrxR has biological functions that are not directly mediated by reduction of Trx.

### Cell Replication

As previously noted, Trx, a physiological substrate of TrxRs, has been shown to play an important role in regulating cell growth and inhibiting apoptosis. See, e.g., Baker, A., Payne, C. M., Briehl, M. M., Powis, G. Cancer Res. 57:5162-5167 (1997). Trx has to be in a reduced form in order to exert these effects, and mutant redox-inactive forms of Trx are unable to stimulate cell growth or inhibit apoptosis.

The only known mechanism for the reduction of Trx is through NADPH-dependent reduction by TrxR. It would be thought, therefore, that TrxRs could also play a role in regulating cell growth. However, TrxR activity in cultured cells can be increased several-fold by including Selenium in the growth medium without a marked effect on the growth rate of cells. See, e.g., Gallegos, A., Berggren, M., Gasdaska, J. R. Powis, G. Cancer Res. 57:4965-4970 (1997). Transfection of MCF-7 breast cancer cells with the TrxR1 variant, Grim-12, results in a greater than 3-fold increase in TrxR activity, but a less than 50% stimulation of cell growth. See, e.g., Hofman, E. R., Boyanapalli, M., et al. Mol. Cell. Biol. 18:6493-6504 (1998). It is possible that the lack of a correlation between increased TrxR activity and cell growth is due to the fact that most cell lines have been selected to grow in Selenium-defecient medium.

In contrast with the lack of effect of increased TrxR activity on cell growth, inhibiting TrxR activity to below normal levels is associated with inhibited cell growth. Several in vitro inhibitors of TrxR have been reported and, although many of these compounds only inhibit the reduced form of TrxR, it is likely that TrxR will be sensitive to these inhibitors in vivo, since TrxR is expected to exist predominantly in the reduced form due to the presence of cytosolic NADPH concentrations that are greater than the Kₘ of TrxR for NADPH. See, e.g., Cromer, S., Arscott, L. D., et al. J. Biol. Chem. 273:20096-20101 (1998). Two such inhibitors of TrxR are the anti-tumour quinones doxorubicin and diaziquone; wherein treatment of cells with either of these compounds leads to secondary inhibition of ribonucleotide reductase and inhibition of cell growth. See, e.g., Hofman, E. R., Boyanapalli, M., et al. Mol. Cell. Biol. 18:6493-6504 (1998).

### p53 Activity

p53 is a tumor-suppressor protein and transcription factor that is deleted in a number of human cancers. See, e.g., Lane, D. P. Br. Med. Bull. 50:582-599 (2004). As in mammalian cells, when wild-type (but not mutant) forms of the human tumor-suppressor p53 gene are expressed in the fusion yeast Schizosaccharomyces pombe, strong growth inhibition occurs. See, e.g., Bischoff, J. R., Casso, D., Beach, D. Mol. Cell. Biol. 12:1405-1411 (1999). Using this as a model system to screen for genes whose function is required for normal activity of p53, a mutant yeast strain was found that was partially resistant to the effects of p53 expression with a recessive mutation in a novel gene (trr1) with strong identity with that encoding TrxR. See, e.g., Casso, D., Beach, D. Mol. Gen. Genet. 252:518-529 (1999). The levels and localization of the p53 protein were unchanged in the mutant yeast strain, suggesting that it was not p53 expression that was altered. Loss of trr1 function resulted in yeast with an increased sensitivity to the toxic effects of H₂O₂ and a 100% oxygen atmosphere. Studies in the budding yeast Saccharomyces cerevisiae have also shown that deletion of the trr1 gene inhibits the ability of human p53 to stimulate reporter gene expression.

Whether TrxR exerts similar control over the function of p53 in mammalian cells is not known. However, it is known that the ability of p53 to bind toDNA is inhibited by oxidizing conditions (see, e.g., Hainaut, P., Milner, J. Cancer Res. 53:4469-4473 (1998)), and p53 expression leads to alterations in the expression of a number of redox genes, including a decrease in TrxR expression (see, e.g., Polyak, K., Xia, Y., et al. Nature (London) 389:300-303 (1997)).

### Protection Against Oxidative Stress

The continual formation of low levels of ROS is part of normal O₂ metabolism; however, increased production of ROS, or a functional decrease in one or more of the protective systems present in the cell, can result in unrepaired macromolecular damage (i.e., oxidation of protein thiols), which may then lead to pathological processes, including apoptosis. See, e.g., Zhivotovsky, B., Orrenius, S., et al. Nature (London) 391:449-450 (1998). Trx has been shown to prevent apoptosis in cells treated with agents known to produce ROS. By way of example, the levels of TrxR-1 mRNA and Trx mRNA are increased in the lungs of newborn baboons exposed to air or O₂ breathing, and increases in TrxR-1 and Trx mRNA are also observed in adult baboon lung explants in response to 95% O₂. It has been suggested that these increases in gene expression for TrxR1 and Trx play a protective role against O₂ breathing in the mammalian lung. There have also been reports that TrxR is highly expressed on the surface of human keratinocytes and melanocytes, where it has been suggested to provide the skin's first line of defence against free radicals generated in response to UV light. See, e.g., Schallreuter, K. U., Wood, J. M. Cancer Lett. 36:297-305 (1997).

### Ascorbate Recycling

Humans lack the ability to synthesize ascorbic acid, an important antioxidant in the protection of cells from oxidative stress; therefore dietary intake and the recycling of ascorbate from its oxidized forms (dehydroascorbic acid and the ascorbyl free radical) are essential for maintenance of in vivo ascorbate levels.

It has been demonstrated that maintenance of rats on a Selenium-deficient diet results in decreased liver ascorbate, glutathione peroxidase, and TrxR levels, while liver glutathione levels are unchanged. See, e.g., May, J. M., Mendiratta, S., et al. J. Biol. Chem. 272:22607-22610 (1997). In another study, treatment of HL-60 cells with buthionine sulphoxamine or diethyl maleate resulted in decreases in cellular glutathione to approximately 10% of that in controls, but had no effect on the ability of these cells to reduce dehydroascorbic acid. See, e.g., Guaiquil, V. H., Farber, C. M., et al. J. Biol. Chem. 272:9915-9921 (1997). TrxR has also been shown to reduce the ascorbyl free radical to ascorbate with a Kₘ of 2.8 μM, which is in the physiological range for this free radical in cells undergoing oxidant stress. See, e.g., May, J. M., Cobb, C. E., et al. J. Biol. Chem. 273:23039-23045 (1998). These studies suggest that, in addition to protecting the cell from oxidative stress by maintaining Trx in its reduced state, TrxR may play an additional role through the recycling of ascorbate.

### Cancer Involvement

It has been suggested, based on purification yields, that the level of TrxR in tumor cells is 10-fold or more greater than in normal tissues. See, e.g., Tamura, T., Stadtman, T. C. Proc. Natl. Acad. Sci. U.S.A. 93:1006-1011 (1996). TrxR has also been reported to be elevated in human primary melanoma and to show a correlation with invasiveness. See, e.g., Fuchs, J. Arch. Dermatol. 124:849-850 (1998).

As previously discussed, the Trx system is as an electron donor for ribonucleotide reducatse, which is frequently greatly over-expressed in cancer cells potentially leading to expanded and inbalanced deoxynucletide pools which are mutagenic, which may accelerate the development of the malignant phenotype by major genetic rearrangements, gene amplifications, total loss of growth control and therapy resistance. It is also not clear whether any of the involvements of the Trx system are obligatory for cancer development, although some results indicate that the Trx system indeed is necessary. However, a significant amount of further research is clearly needed in order to ascertain the importance of the Trx system in cancer progress. Nonetheless, it is clearly evident that the Trx system plays a central role in established cancers particularly for distant metastasis and angiogenesis. A recent study utilizing TrxR-1 knock-down in tumor cells intriguingly demonstrated a necessity of TrxR-1 expression for cancer cell growth and tumor development. See, e.g., Yoo, M. H., Xu, X. M., et al. Thioredoxin reductase 1 deficiency reverses tumor phenotype and tumorigenicity of lung carcinoma cells. J. Biol. Chem. 281:13005-13008 (2006).

B. Thioredoxin (Trx)

Thioredoxins (Trxs) are proteins that act as antioxidants by facilitating the reduction of other proteins by cysteine thiol-disulfide exchange. While glutaredoxins mostly reduce mixed disulfides containing glutathione, thioredoxins are involved in the maintenance of protein sulfhydryls in their reduced state via disulfide bond reduction. See, e.g., Print, W. A., et al. The role of the thioredoxin and glutaredoxin pathways in reducing protein disulfide bonds in the Escherichia coli cytoplasm. J. Biol. Chem. 272:15661-15667 (1996). Thiol-disulfide exchange is a chemical reaction in which a thiolate group (S⁻) attacks a sulfur atom of a disulfide bond (—S—S—). The original disulfide bond is broken, and its other sulfur atom is released as a new thiolate, thus carrying away the negative charge. Meanwhile, a new disulfide bond forms between the attacking thiolate and the original sulfur atom. The transition state of the reaction is a linear arrangement of the three sulfur atoms, in which the charge of the attacking thiolate is shared equally. The protonated thiol form (—SH) is unreactive (i.e., thiols cannot attack disulfide bonds, only thiolates). In accord, thiol-disulfide exchange is inhibited at low pH (typically, <8) where the protonated thiol form is favored relative to the deprotonated thiolate form. The pKₐ of a typical thiol group is approximately 8.3, although this value can vary as a function of the environment. See, e.g., Gilbert, H. F., Molecular and cellular aspects of thiol-disulfide exchange. Adv. Enzymol. 63:69-172 (1990); Gilbert, H. F., Thiol/disulfide exchange equilibria and disulfide bond stability. Meth. Enzymol. 251:8-28 (1995).

Thiol-disulfide exchange is the principal reaction by which disulfide bonds are formed and rearranged within a protein. The rearrangement of disulfide bonds within a protein generally occurs via intra-protein thiol-disulfide exchange reactions; a thiolate group of a cysteine residue attacks one of the protein's own disulfide bonds. This process of disulfide rearrangement (known as disulfide shuffling) does not change the number of disulfide bonds within a protein, merely their location (i.e., which cysteines are actually bonded). Disulfide reshuffling is generally much faster than oxidation/reduction reactions, which actually change the total number of disulfide bonds within a protein. The oxidation and reduction of protein disulfide bonds in vitro also generally occurs via thiol-disulfide exchange reactions. Typically, the thiolate of a redox reagent such as glutathione or dithiothreitol (DTT) attacks the disulfide bond on a protein forming a mixed disulfide bond between the protein and the reagent. This mixed disulfide bond when attacked by another thiolate from the reagent, leaves the cysteine oxidized. In effect, the disulfide bond is transferred from the protein to the reagent in two steps, both thiol-disulfide exchange reactions.

Thioredoxin (Trx) was originally described in 1964 as a hydrogen donor for ribonucleotide reductase which is an essential enzyme for DNA synthesis in Escherichia coli. Human thioredoxin was originally cloned as a cytokine-like factor named adult T cell leukemia (ATL)-derived factor (ADF), which was first defined as an IL-2 receptor α-chain (IL-2Ra, CD25)-inducing factor purified from the supernatant of human T cell leukemia virus type-1 (HTLV-1)-transformed T cell ATL2 cells. See, e.g., Yordi, J., et al. ADF, a growth-promoting factor derived from adult T cell leukemia and homologous to thioredoxin: possible involvement of dithiol-reduction in the IL-2 receptor induction. EMBO J. 8:757-764 (1989).

Proteins sharing the highly conserved -Cys-Xxx-Xxx-Cys- and possessing similar three-dimensional structure (i.e., the thioredoxin fold) are classified as belonging to the thioredoxin family. In the cytosol, members of the thioredoxin family include: the “classical cytosolic” thioredoxin 1 (Trx-1) and glutaredoxin 1. In the mitochondria, family members include: mitochondrial-specific thyroxin 2 (Trx-2) and glutaredoxin 2. Thioredoxin family members in the endoplasmic reticulum (ER) include: protein disulfide isomerase (PDI); calcium-binding protein 1 (CaBP1); ERp72; Trx-related transmembrane protein (TMX); ERdj5; and similar proteins. Macrophage migration inhibitory factor (MIF) is a pro-inflammatory cytokine which was originally described as a soluble factor expressed by activated T cells in delayed-type hypersensitivity. See, e.g., Morand, E. F., et al. MIF: a new cytokine link between rheumatoid arthritis and atherosclerosis. Nat. Rev. Drug Discov. 5:399-411 (2006). MIF also possesses a redox-active catalytic site and exhibits disulfide reductase activity. See, e.g., Kleeman, R., et al. Disulfide analysis reveals a role for macrophage migration inhibitory factor (MIF) as thiol-protein oxidoreductase. J. Mol. Biol. 280:85-102 (1998). MIF has pro-inflammatory functions, whereas thioredoxin 1 (TX-1) exhibits both anti-inflammatory and anti-apoptotic functions. Trx-1 and MIF control their expression reciprocally, which may explain their opposite functions. However, Trx-1 and MIF also share various similar characteristics. For example, both have a similar molecular weight of approximately 12 kDa and are secreted by a leaderless export pathway. They both share the same interacting protein such as Jun activation domain-binding protein 1 (JABI) in cells. Glycosylation inhibitory factor (GIF), which was originally reported as a suppressive factor for IgE response, is a posttranslationally-modified MIF with cysteinylation at Cys60. The biological difference between MIF and GIF may be explained by redox-dependent modification, possibly involving Trx-1. See, e.g., Nakamura, H., Thioredoxin and its related molecules: update 2005. Antioxid. Redox Signal. 7:823-828 (2005).

The mammalian thioredoxins (Trxs) are a family of 10-12 kDa proteins that contain a highly conserved -Trp-Cys-Gly-Pro-Cys-Lys-catalytic site. See, e.g., Nishinaka, Y., et al. Redox control of cellular functions by thioredoxin: A new therapeutic direction in host defense. Arch. Immunol. Ther. Exp. 49:285-292 (2001). The active site sequences is conserved from Escherichia coli to humans. Thioredoxins in mammalian cells possess >90% homology and have approximately 27% overall homology to the E. coli protein.

As previously discussed, the thioredoxins act as oxidoreductases and undergo reversible oxidation/reduction of the two catalytic site cysteine (Cys) amino acid residues. The most prevalent thioredoxin, Trx-1, is involved in a plethora of diverse biological activities. The reduced dithiol form of Trx [Trx-(SH)₂] reduces oxidized protein substrates that generally contain a disulfide group; whereas the oxidized disulfide form of Trx [Trx-(SS)] redox cycles back in an NADPH-dependent process mediated by thioredoxin reductase (TrxR), a homodimer comprised of two identical subunits each having a molecular weight of approximately 55 kDa. The conversion of thioredoxin from the disulfide form (oxidized) to the dithiol form (reduced) is illustrated in the diagram, below:

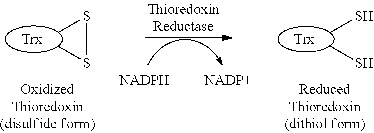

Two principal forms of thioredoxin (Trx) have been cloned. Trx-1 is a 105-amino acid protein. In almost all (>99%) of the human form of Trx-1, the first methionine (Met) residue is removed by an N-terminus excision process (see, e.g., Giglione, C., et al. Protein N-terminal methionine excision. Cell. Mol. Life Sci. 61:1455-1474 (2004), and therefore the mature protein is comprised of a total of 104-amino acid residues from the N-terminal valine (Val) residue. Trx-1 is typically localized in the cytoplasm, but it has also been identified in the nucleus of normal endometrial stromal cells, tumor cells, and primary solid tumors. Various types of post-translational modification of Trx-1 have been reported: (i) C-terminal truncated Trx-1, comprised of 1-80 or 1-84 N-terminal amino acids, is secreted from cells and exhibits more cytokine-like functions than full-length Trx-1; (ii) S-Nitrosylation at Cys69 is important for anti-apoptotic effects; (iii) glutathionylation occurs at Cys73, which is also the site responsible for the dimerization induced by oxidation; (iv) in addition to the original active site between Cys32 Cys35, another dithiol/disulfide exchange is observed between and Cys62 and Cys69, allowing intramolecular disulfide formation; and (v) Cys35 and Cys69 are reported to be the target for 15-deoxyprostaglandin-J₂. See, e.g., Nakamura, H. Thioredoxin and its related molecules: update 2005. Antioxid. Redox Signal. 7:823-828 (2005).

Reduced Trx-1, but not its oxidized form or a Cys→Ser catalytic site mutant, has been shown to bind to various intracellular proteins and may regulate their biological activities. In addition to NK-κB and Ref-1, Trx-1 binds to various isoforms of protein kinase C (PKC); p40 phagocyte oxidase; the nuclear glucocorticoid receptor; and lipocalin. Trx-1 also binds to apoptosis signal-regulating kinase 1 (ASK 1) in the cytosol under normal physiological conditions. However, when Trx-1 becomes oxidized under oxidative stress, ASK 1 is dissociated from Trx-1, thus causing Trx-1 to become a homodimer which transduces the apoptotic signal. ASK 1 is an activator of the JNK and p38 MAP kinase pathways, and is required for TNFα-mediated apoptosis. See, e.g., Saitoh, M., et al. Mammalian thioredoxin is a direct inhibitor of apoptosis signal-regulating kinase 1 (ask1). EMBO J. 17:2596-2606 (1998).

Another binding protein for Trx-1 is thioredoxin-binding protein 2 (TBP-2) which is identical to Vitamin D₃ upregulating protein 1 (VDUP1). TBP-2/VDUP1 was originally reported as the product of a gene whose expression was upregulated in HL-60 cells stimulated with 1a, 25-dihydroxyvitamin D₃. The interaction of TBP-2/VDUP1 with Trx was observed both in vitro and in vivo. TBP-2/VDUP1 only binds to the reduced form of Trx and acts as an apparent negative regulator of Trx. See, e.g., Nishiyama, A., et al. Identification of thioredoxin-binding protein-2/Vitamin D(3) up-regulated protein 1 as a negative regulator of thioredoxin function and expression. J. Biol. Chem. 274:21645-21650 (1999). Although the mechanism is unknown, a reciprocal expression pattern of Trx and TBP-2 was often reported upon various types of stimulation. Several highly homologous genes of TBP-2/VDUP1 have been indentified. A TBP-2 homologue, TBP-2-like inducible membrane protein (TLIMP) is a novel VD3 or peroxisome proliferator-activated receptor-γ (PPAR-γ) ligand-inducible membrane-associated protein and plays a regulatory role in cell proliferation and PPAR-γ activation. See, e.g., Oka, S., et al. Thioredoxin-binding protein 2-like inducible membrane protein is a novel Vitamin D₃ and peroxisome proliferator-activated receptor (PPAR) gamma ligand target protein that regulates PPAR gamma signaling. Endocrinology 147:733-743 (2006). Another TBP-2 homologous gene, DRH1, is reported to be down-regulated in hepatocellular carcinoma. See, e.g., Yamamoto, Y., et al. Cloning and characterization of a novel gene, DRH1, down-regulated in advanced human hepatocellular carcinoma. Clin. Cancer Res. 7:297-303 (2001). These results indicate that the familial members of TBP-2 may also play a role in cancer suppression.

TBP-2 also possesses a growth suppressive activity. Overexpression of TBP-2 was shown to resulted in growth suppression. TBP-2 expression is upregulated by Vitamin D₃ treatment and serum- or IL-2-deprivation, thus leading to growth arrest. TBP-2 is found predominantly in the nucleus. TBP-2 mRNA expression is down-regulated in several tumors (see, e.g., Butler, L. M., et al. The histone deacetylase inhibitor SAHA arrests cancer cell growth, up-regulates thioredoxin-binding protein-2 and down-regulates thioredoxin. Proc. Natl. Acad. Sci. USA 99:11700-11705 (2002)) and lymphoma (see, e.g., Tome, M. E., et al. A redox signature score indentifies diffuse large B-cell lymphoma patients with poor prognosis. Blood 106:3594-3601 (2005)), suggesting a close association between the expression reduction and tumorigenesis. TBP-2 expression is also downregulated in melanoma metastasis. See, e.g., Goldberg, S. F., et al. Melanoma metastasis suppression by chromosome 6: evidence for a pathway regulated by CRSP3 and TXNIP. Cancer Res. 63:432-440 (2003).

Loss of TBP-2 seems to be an important step of human T cell leukemia virus 1 (HTLV-1) transformation. In an in vitro model, HTLV-1-infected T-cells required IL-2 to proliferate in the early phase of transformation, but subsequently lost cell cycle control in the late phase, as indicated by their continuous proliferative state in the absence of IL-2. The change of cell growth phenotype has been suggested to be one of the oncogenic transformation processes. See, e.g., Maeda, M., et al. Evidence for the interleukin-2 dependent expansion of leukemic cells in adult T cell leukemia. Blood 70:1407-1411 (1987). The expression of TBP-2 is lost in HTLV-I-positive IL-2-independent T cell lines (due to the DNA methylation and histone deacetylation); but is maintained in HTLV-I-positive IL-2-dependent T cell lines, as well as in HTLV-1-negative T cell lines. See, e.g., Ahsan, M. K., et al. Loss of interleukin-2-dependancy in HTLV-1-infected T cells on gene silencing of thioredoxin-binding protein-2. Oncogene 25:2181-2191 (2005). Additionally, the murine knock-out HcB-19 strain, which has a spontaneous mutation in TBP-2/Txnip/VDUP1 gene, has been reported to have an increased incidence of hepatocellular carcinoma (HCC), showing that TBP-2/VDUP1 is a potential tumor suppressor gene candidate, in vivo. See, e.g., Sheth, S. S., et al. Thioredoxin-interacting protein deficiency disrupts the fasting-feeding metabolic transition. J. Lipid Res. 46:123-134 (2005). The same HcB-19 mice also exhibited decreased NK cells and reduced tumor rejection. TBP-2 was also found to interact with various cellular target such as JAB1 and FAZF, and may be a component of a transcriptional repressor complex. See, e.g., Lee, K. N., et al. VDUP1 is required for the development of natural killer cells. Immunity 22:195-208 (2005). However, the precise mechanism of its molecular action remains to be elucidated.

Trx-2 is a 166-amino acid residue protein that contains a 60-amino acid residue N-terminal translocation sequence that directs it to the mitochondria. See, e.g., Spyroung, M., et al. Cloning and expression of a novel mammalian thioredoxin. J. Biol. Chem. 272: 2936-2941 (1997). Trx-2 is expressed uniquely in mitochondria, where it regulates the mitochondrial redox state and plays an important role in cell proliferation. Trx-2-deficient cells fall into apoptosis via the mitochondria-mediated apoptosis signaling pathway. See, e.g., Noon, L., et al. The absence of mitochondrial thioredoxin-2 causes massive apoptosis and early embryonic lethality in homozygous mice. Mol. Cell. Biol. 23:916-922 (2003). Trx-2 was found to form a complex with cytochrome c localized in the mitochondrial matrix, and the release of cytochrome c from the mitochondria was significantly enhanced when expression of Trx-2 was inhibited. The overexpression of Trx-2 produced resistance to oxidant-induced apoptosis in human osteosarcoma cells, indicating a critical role for the protein in protection against apoptosis in mitochondria. See, e.g., Chen, Y., et al. Overexpressed human mitochondrial thioredoxin confers resistance to oxidant-induced apoptosis in human osteosarcoma cells. J. Biol. Chem. 277:33242-33248 (2002).

As both Trx-1 and Trx-2 are known regulators of the manifestation of apoptosis under redox-sensitive capases, their actions may be coordinated. However, the functions of Trx-1 and Trx-2 do not seem to be capable of compensating for each other completely, since Trx-2 knockout mice were found be embryonically lethal. See, e.g., Noon, L., et al. The absence of mitochondrial thioredoxin-2 causes massive apoptosis and early embryonic lethality in homozygous mice. Mol. Cell. Biol. 23:916-922 (2003). Moreover, the different subcellular locations of both the thioredoxin reductase (TrxR) and thioredoxin (Trx) subtypes suggest that the cytoplasmic and mitochondrial systems may play different roles within cells. See, e.g., Powis, G. and Monofort, W. R. Properties and biological activities of thioredoxins. Ann. Rev. Pharmacol. Toxicol. 41:261-295 (2001).

### Biological Activities of the TrxR/Trx System

### Physiological and Effects Modulated by Thioredoxin (Trx) and Related Proteins

Mammalian cells contain a glutathione (GSH)/glutaredoxin system and a thioredoxin(Trx)/thioredoxin reductase (TrxR) system as the two major antioxidant systems. The intracellular concentration of GSH is approximately 1-10 mM in mammalian cells, whereas the normal reported intracellular concentration of Trx is approximately 0.1-2 μM. Accordingly, Trx may initially appear as a minor component as an intracellular antioxidant. However, Trx is a major enzyme supplying electrons to peroxiredoxins or methionine sulfoxide reductases, and acts as general protein disulfide reductase. Trx knock-out mice are embryonic lethal (see, e.g., Matsui, M., et al. Early embryonic lethality caused by targeted disruption of the mouse thioredoxin gene. Dev. Biol. 178:179-185 (1996)), thus illustrating that the Trx/TrxR system is playing an essential survival role in mammalian cells. This importance may be explained by Trx playing a crucial role in the interaction with specific target molecules including, but not limited to, the inhibition of apoptosis signal regulation kinase I (ASK1) activation (see, e.g., Saitoh, M., et al. Mammalian thioredoxin is a direct inhibitor of apoptosis signal-regulation kinase 1 (ASK1). EMBO J. 17:2596-2606 (1998)) and in the regulation of DNA binding activity of transcriptional factors such as AP-1, NF-κB and p53 for the transcriptional control of essential genes (see, e.g., Nakamura, H., et al. Redox regulation of cellular activation. Ann. Rev. Immunol. 15:351-369 (1997)). For example, during oxidative stress Trx-1 translocates from the cytosol into the nucleus where it augments DNA-binding activity of these aforementioned transcriptional factors. Alternately, the role of Trx in the defense against cellular oxidative stress or to supply the “building blocks” for DNA synthesis, via ribonucleotide reductase, is equally essential. Trx-1 and the 14 Kda Trx-like protein (TRP14) reactivates PTEN (a protein tyrosine phosphatase which reverses the action of phosphoinositide-3-kinase) by the reduction of the disulfide which is reversibly induced by hydrogen peroxide. See, e.g., Jeong, W., et al. Identification and characterization of TRP14, a thioredoxin-related protein of 14 Kda. J. Biol. Chem. 279:3142-3150 (2004). Exogenous Trx-1 has been shown to be capable of entering cells and attenuate intracellular reactive oxygen species (ROS) generation and cellular apoptosis. See, e.g., Kondo, N., et al. Redox-sensing release of human thioredoxin from T lymphocytes with negative feedback loops. J. Immunol. 172:442-448 (2004). Additionally, HMG-CoA reductase inhibitors (commonly utilized for the prevention of atherosclerosis) have also been shown to augment S-Nitrosylation of Trx-1 at Cys⁶⁹ and reduce oxidative stress. See, e.g., Haendeler, J., et al. Antioxidant effects of statins via S-nitrosylation and activation of thioredoxin in endothelial cells. Circulation 110:856-861 (2004).

### The Thioredoxin (Trx)/Thioredoxin Reductase (TrxR) System as a Cofactor in DNA Synthesis

The Trx/TrxR-coupled system plays a critical role in the generation of deoxyribonucleotides which are needed in DNA synthesis and essential for cell proliferation. Trx provides the electrons needed in the reduction of ribose by ribonucleotide reductase, an enzyme that catalyzes the conversion of nucleotide diphosphates into deoxyribonucleotides. Ribonucleotide reductase is necessary for DNA synthesis and cell proliferation. Diaziquone and doxorubicin have been shown to inhibit the Trx/TrxR system resulting in a concentration-dependent inhibition of cellular ribonucleotide reductase activity in human cancer cells. See, e.g., Mau, B., et al. Inhibition of cellular thioredoxin reductase by diaziquone and doxorubicin. Biochem. Pharmacol. 43:1621-1626 (1992). Similarly, the glutaredoxin/glutathione-coupled reaction also provides reducing equivalents for ribonucleotide reductase. For example, depletion of glutathione has been shown to inhibit DNA synthesis and induce apoptosis in a number of cancer cell lines. See, e.g., Dethlefsen, L. A., et al. Toxic effects of acute glutathione depletion by on murine mammary carcinoma cells. Radiat. Res. 114:215-224 (1988).

### The Role of the Thioredoxin (Trx)/Thioredoxin Reductase (TrxR) System in Cellular Apoptosis

Trx-1 was shown to prevent apoptosis (programmed cell death) when added to the culture medium of lymphoid cells or when its gene is transfected into these cells. Murine WEH17.2 lymphoid cells underwent apoptosis when exposed to the glucocorticoid dexamethasone or the topoisomerase I inhibitor etoposide and, to a lesser extent, when exposed to the kinase inhibitor staurosporine or thapsigarin, an inhibitor of intracellular calcium uptake. See, e.g., Powis, G., et al. Thioredoxin control of cell growth and death and the effects of inhibitors. Chem. Biol. Interact. 111:23-34 (1998). Trx levels in the cytoplasm and nucleus were increased following stable transfection of these cells with human Trx-1, and as a result the transfected cells showed resistance to apoptosis when exposed to dexamethasone and the other cytotoxic agents. The pattern of apoptosis inhibition with Trx-1 transfection was similar to that following transfection with the bcl-2 anti-apoptotic oncogene. In cooperation with redox factor-1, Trx-1 induces p53-dependent p-21 transactivation leading to cell-cycle arrest and DNA repair. See, e.g., Ueda, S., et al. Redox control of cell death. Antioxid. Redox Signal. 4:405-414 (2002). In addition, Trx-1 regulates the signaling for apoptosis by suppressing the activation of apoptosis signal-regulation kinase-1 (ASK-1). See, e.g., Nakamura, H., et al. Redox regulation of cellular activation. Ann. Rev. Immunol. 15:351-369 (1997).

The specific mechanism(s) by which Trx-2 imparts resistance to chemotherapy apoptosis in cancer cells has not been fully elucidated. Based on the current studies, one may postulate, however, that it appears increases in cellular reductive power allows ongoing protective and/or reparative reduction of proteins, DNA, cell membranes or carbohydrates that have been damaged or would otherwise be damaged by oxidative chemical species, thus counteracting of the induced cellular apoptosis from the chemotherapy and/or radiation therapy. The analogous glutaredoxin/glutathione system may also prevent apoptosis. In either instance, there is a lack of apoptotic sensitivity to normal treatment interventions that appears to be mediated by the increased Trx-2 and by glutaredoxin pathways. In the glutaredoxin mediated pathway, as an example, glutathione depletion with L-buthionine sulfoximine was shown to inhibit the growth of several breast and prostate cancer cell lines, and in rat R3230Ac mammary carcinoma cells, it markedly increased apoptosis. It is thought that mitochondrial swelling following depletion of glutathione may be the stimulus for apoptosis in these cells. See, e.g., Bigalow, J. E., et al. Glutathione depletion or radiation treatment alters respiration and induces apoptosis in R3230Ac mammary carcinoma. Adv. Exp. Med. Biol. 530:153-164 (2003). Trx-2 has been shown to be a critical regulator of mitochondrial cytochrome c release and apoptosis. See, e.g., Tanaka, M., et al. Thioredoxin-2 (TX-2) is an essential gene in regulating mitochondrial-dependent apoptosis. EMBO J. 21:1695-1701 (2002).

### The Role of Thioredoxin (Trx) in Stimulating Angiogenesis

Angiogenesis by cancer cells provides a growth and survival advantage that is localized to the primary as well as secondary (metastatic tumors). Malignant tumors are generally poorly vascular, however, with overexpression of angiogenesis factors, the tumor cells gain better nutrition and oxygenation, thereby promoting proliferation of cancer cells and growth of the tumor. Transfection of several different cell lines, including human breast cancer MCF-7, human colon cancer HT29, and murine WEHI7.2 lymphoma cells, with human Trx-1 produced significant increases in secretion of vascular endothelial growth factor (VEGF). See, e.g., Welch, S. J., et al. The redox protein thioredoxin-1 increases hypoxia-inducible factor 1α protein expression: Trx-1 overexpression results in increased vascular endothelial growth factor production and enhanced tumor angiogenesis. Cancer Res. 62:5089-5095 (2003). VEGF secretion was increased by 41%-77% under normoxic (20% oxygen) conditions and by 46%-79% under hypoxic (1% oxygen) conditions. In contrast, transfection with a redox-inactive Trx mutant (Cys→Ser) partially inhibited VEGF production. When Trx-1-transfected WEH17.2 cells were grown in SCID mice, VEGF levels were markedly increased and tumor angiogenesis (as measured by microvessel vascular density) was also increased by 2.5-fold, relative to wild-type WEH17.2 tumors. Id. Accordingly, there is evidence that the thioredoxin system can increase VEGF levels in cancer cells.

### Role of Thioredoxin (Trx) in Stimulating Cell Proliferation

Exposure to Trx-1 was shown to stimulate the growth of lymphocytes, fibroblasts, and a variety of leukemic and solid tumor cell lines. See, e.g., Powis, G. and Monofort, W. R. Properties and biological activities of thioredoxins. Ann. Rev. Pharmacol. Toxicol. 41:261-295 (2001). In contrast, the previously discussed Cys→Ser redox mutant at 50-fold higher concentrations, did not stimulate cell growth. While the mechanisms for this proliferative effect are not fully elucidated, there is evidence that such Trx-mediated increases in cell proliferation are multifactorial, and are related to both the increased production of various cytokines (e.g., IL-1, IL-2, and tumor necrosis factor α (TNFα)) and the potentiation of growth factor activity (e.g., basic fibroblast growth factor (bFGF)). Additionally, there is thought to also be increased DNA synthesis and transcription, as well.

### The Antioxidant Effects of Thioredoxin (Trx)

Glutathione peroxidase and membrane peroxidases play a highly important role in protecting cells against the damaging effects of reactive oxygen species (ROS) including, but not limited to, oxygen radicals and peroxides. See, e.g., Bigalow, J. E., et al. The importance of peroxide and superoxide in the x-ray response. Int. J. Radiat. Oncol. Biol. Phys. 22:665-669 (1992). These enzymes utilize use thiol groups as an electron source for scavenging reactive oxygen species (ROS), and in the process, form homo- or heterodimers with other peroxidases through the formation of disulfide bonds with conserved cysteine residues. Trx produces antioxidant effects primarily by serving as an electron donor for thioredoxin peroxidases. Accordingly, by the reduction of oxidized peroxidases, Trx restores the enzyme to its monomeric form, which allows the enzyme to continue its oxyradical scavenging.

Trx may also increase the expression of thioredoxin peroxidase. For example, in MCF-7 human breast cancer cells stably transfected with Trx-1, mRNA for thioredoxin peroxidase was doubled relative to wild-type and empty-vector transformed cells, and Western blots showed increased protein levels as well. Moreover, Trx-1 transfected murine WEH17.2 cells were more resistant to peroxide-induced apoptosis than wild-type and empty-vector transformed cells. However, Trx-1 transfection did not protect the cells from apoptosis induced by dexamethasone or chemotherapeutic agents. See, e.g., Berggren, M. I., et al. Thioredoxin peroxidase-1 is increase in thioredoxin-1 transfected cells and results in enhanced protection against apoptosis caused by hydrogen peroxide, but not by other agents including dexamethasone, etoposide, and deoxorubin. Arch. Biochem. Biophys. 392:103-109 (2001).

### The Role of Thioredoxin (Trx) in Stimulating Transcription Factor Activity

Thioredoxin (Trx) increases the DNA-binding activity of a number of transcription factors (e.g., NF-κB, AP-1, and AP-2) and nuclear receptors (e.g., glucocorticoid and estrogen receptors). See, e.g., Nishinaka, Y., et al. Redox control of cellular functions by thioredoxin: A new therapeutic direction in host defense. Arch. Immunol. Ther. Exp. 49:285-292 (2001). By way of non-limiting example, with regard to NF-κB, Trx reduces the Cys residue of the p50 subunit in the nucleus, thus allowing it to bind to DNA. See, e.g., Mau, B., et al. Inhibition of cellular thioredoxin reductase by diaziquone and doxorubicin. Biochem. Pharmacol. 43:1621-1626 (1992). In the cytoplasm, however, Trx paradoxically interferes with NF-κB by blocking dissociation of the endogenous inhibitor IκB and interfering with signaling to IκB kinases. See, e.g., Hirota, K., et al. Distinct roles of thioredoxin in the cytoplasm and in the nucleus: A two-step mechanism of redox regulation of transcription factor nf-κB. J. Biol. Chem. 274:27891-27897 (1999). The effect of Trx on some transcription factors is mediated via reduction of Ref-1, a 37 kDa protein that also possesses DNA-repair endonuclease activity. For example, Trx reduces Ref-1, which in turn reduces cysteine residues within the fos and jun subunits of AP-1 to promote DNA binding. The redox activity of Ref-1 is found in its N-terminal domain, whereas its DNA repair activity is located among C-terminal sequences.

### Thioredoxin (Trx) Binding to Cellular Proteins

Reduced Trx-1, but not its oxidized form or a catalytic site Cys→Ser redox inactive mutant, binds to a variety of cellular proteins and may regulate their biological activities. See, e.g., Powis, G. and Monofort, W. R. Properties and biological activities of thioredoxins. Ann. Rev. Pharmacol. Toxicol. 41:261-295 (2001). In addition, to NK-κB and Ref-1, Trx binds to: (i) apoptosis signal-regulating kinase 1 (ASK1), (ii) various isoforms of protein kinase C (PKC), (iii) p40 phagocyte oxidase, (iv) the nuclear glucocorticoid receptor, and (v) lipocalin. ASK1, for example, is an activator of the JNK and p38 MAP kinase pathways and is required for TFNα-mediated apoptosis. See, e.g., Ichijo, H., et al. Induction of apoptosis by ask1, a mammalian map kinase that activates jnk and p38 signaling pathways. Science 275:90-94 (1997). Trx binds to a site at the N-terminal of ASK1, thus inhibiting the kinase activity and blocking ASK1-mediated apoptosis. See, e.g., Saitoh, M., et al. Mammalian thioredoxin is a direct inhibitor of apoptosis signal-regulation kinase 1 (ask1). EMBO J. 17:2596-2606 (1998). Under conditions of oxidative stress, however, reactive oxygen species are produced that oxidize the Trx, thus promoting its dissociation from ASK1 and leading to the concomitant activation of ASK1.

### The Role of Thioredoxin (Trx) in Stimulating Hypoxia-Inducible Factor (HIF)

Cancer cells are able to adapt to the hypoxic conditions found in nearly all solid tumors. Hypoxia leads to activation of hypoxia-inducible factor 1 (HIF-I), which is a transcription factor involved in development of the cancer phenotype. Specifically, HIF binds to hypoxia response elements (HRE) and induces expression of a variety of genes that serve to promote: (i) angiogenesis (e.g., VEGF); (ii) metabolic adaptation (e.g., GLUT transporters, hexokinase, and other glycolytic enzymes); and (iii) cell proliferation and survival. HIF is comprised of two subunits: (i) HIF-1α (that is induced by hypoxia); and (ii) HIF-1β (that is expressed constitutively). Trx overexpression has been shown to significantly increase HIF-1α under both normoxic and hypoxic conditions, and this was associated with increased HRE activity demonstrated in a luciferase reporter assay as well as increased expression of HRE-regulated genes. HIF may provide tumor cells with a survival advantage under hypoxic conditions by inducing hexokinase and thus allowing glycolysis to serve as the predominant energy source. For example, surgical specimens from patients with metastatic liver cancer had fewer tumor blood vessels and higher hexokinase expression than specimens from hepatocellular carcinoma patients. Hexokinase expression was correlated with HIF-1α expression in both populations, and they co-localized in tumor cells found near necrotic regions.

### Targeting Thioredoxin (Trx)/Thioredoxin Reductase (TrxR)-Coupled Reactions

The biological activities of Trx/TrxR and their apparent relevance to aggressive tumor growth suggest that this system may be an attractive target for cancer therapy. Either individual enzymes or substrates can be altered. In cells that do not contain glutaredoxin, depletion of hexose monophosphate shunt (HMPS)-generated NADPH or, alternately, direct interaction with Txr or TrxR may prove to be viable approaches to blocking HMPS/Trx/TrxR-coupled reactions. In cells where glutaredoxin is present, its reducing activity also may need to be targeted through depletion of glutathione.

### Thioredoxin (Trx) in Plasma or Serum as an Oxidative Metabolism Biological Marker

Thioredoxin 1 (Trx-1) is released by cells in response to changes in oxidative metabolism. See, e.g., Kondo N, et al. Redox-sensing release of human thioredoxin from T lymphocytes with negative feedback loops. J. Immunol. 172:442-448 (2004). Plasma or serum levels of Trx are measurable by a sensitive sandwich enzyme-linked immunosorbent assay (ELISA). Serum plasma levels of Trx are good markers for changes in oxidative metabolism in a variety of disorders. See, e.g., Burke-Gaffney, A., et al. Thioredoxin: friend or foe in human diseases? Trends Pharmacol. Sci. 26:398-404 (2004). For example, plasma levels of Trx are elevated in patients with acquired immunodeficiency syndrome (AIDS) and negatively correlated with the intracellular levels of GSH, suggesting that the HIV-infected individuals with AIDS. See, e.g., Nakamura, H., et al. Elevation of plasma thioredoxin levels in HIV-infected individuals. Int. Immunol. 8:603-611 (1996). In patients with type C chronic hepatitis, serum levels of Trx and ferritin are good markers for the efficacy of interferon therapy. See, e.g., Sumida, Y., et al. Serum thioredoxin levels as an indicator of oxidative stress in patients with hepatitis C virus infection. J. Hepatol. 33:616-622 (2001). In the case of cancer, serum levels of Trx are elevated in patients with hepatocellular carcinoma (see, e.g., Miyazaki, K., et al. Elevated serum levels of serum thioredoxin in patients with hepatocellular carcinoma. Biotherapy 11:277-288 (1998)) and pancreatic cancer (see, e.g., Nakmura, H., et al. Expression of thioredoxin and glutaredoxin, redox-regulating proteins, in pancreatic cancer. Cancer Detect. Prev. 24:53-40 (2000)). The serum levels of Trx decrease after the removal of the main tumor, suggesting that cancer tissues are the main source of the elevated Trx in serum. See, e.g., Miyazaki, K., et al. Elevated serum levels of serum thioredoxin in patients with hepatocellular carcinoma. Biotherapy 11:277-288 (1998).

### Involvement of the Thioredoxin (Trx)/Thioredoxin Reductase (TrxR) System in Cancer

As previously discussed, Trx itself is not mutagenic but rather the Trx system is involved in antioxidant defense and probably in prevention of cancer via the removal of carcinogenic oxidants or by repair of oxidized proteins. Similarly repair of mutagenic DNA lesions by Trx system-dependent nucleotide excision repair and ribonucleotide reductase may protect from cancer. In theory, the Trx system as an electron donor for ribonucleotide reducatse, which is often greatly over-expressed in cancer cells. This over-expression may potentially lead to an expanded and inbalanced deoxynucletide pools which is mutagenic and may accelerate the development of the malignant phenotype by major genetic rearrangements, gene amplifications, total loss of growth control, and resistance to the selected therapy. It is also not clear whether any of the involvements of the Trx system are obligatory for cancer development, although some results indicate that the Trx system indeed is necessary. A significant amount of further research is clearly needed in order to ascertain the importance of the Trx system in cancer progress. Nonetheless, it is evident that the Trx system plays a central role in established cancers particularly for distant metastasis and angiogenesis. A recent study utilizing TrxR-1 knockdown in tumor cells intriguingly demonstrated a necessity of TrxR-1 expression for cancer cell growth and tumor development. See, e.g., Yoo, M. H., Xu, X. M., et al. Thioredoxin reductase 1 deficiency reverses tumor phenotype and tumorigenicity of lung carcinoma cells. J. Biol. Chem. 281:13005-13008 (2006). At present, it is not known which of the function(s) of TrxR-1 and/or the Trx system that are required for cancer development, but it may clearly be context dependent.

Various extracellular roles of thioredoxin (Trx) have been examined in cancer. As previously described, Trx was originally cloned as a cytokine-like factor named ADF. Independently, Trx was also identified as an autocrine growth factor named 3B6-IL1 produced by Epstein-Barr virus-transformed B cells (see, e.g., Wakasugi, H., et al. Epstein-Barr virus-containing B-cell line produces an interleukin 1 that it uses as a growth factor. Proc. Natl. Acad. Sci. USA 84:804-808 (1987)) or as a B cell growth factor named MP6-BCGF produced by the T cell hybridoma MP6 (see, e.g., Rosen A, et al. A CD4+ T cell line-secreted factor, growth promoting for normal and leukemic B cells, identified as thioredoxin. Int. Immunol. 7:625-33 (1995)). Moreover, eosinophil cytotoxicity-enhancing factor (ECEF) was found as a truncated form of Trx (i.e., Trx80) comprising which is the N-terminal 1-80 (or 1-84) residues of Trx (see, e.g., Silberstein, D. S., et al. Human eosinophil cytotoxicity-enhancing factor. Eosinophil-stimulating and dithiol reductase activities of biosynthetic (recombinant) species with COOH-terminal deletions. J. Biol. Chem. 268:913-942 (1993)) and a component of “early pregnancy factor” which was an immunosuppressive factor in pregnant female serum was also identified as Trx (see, e.g., Clarke, F. M., et al. Identification of molecules involved in the “early pregnancy factor” phenomenon. J. Reprod. Fertil. 93:525-539 (1991)). These historical reports, collectively, illustrate that Trx has various important extracellular functions.

Thioredoxin (Trx) expression is frequently markedly increased in a variety of human malignancies including, but not limited to, lung cancer, colorectal cancer, cervical cancer, hepatic cancer, pancreatic cancer, and adenocarcinoma. See, e.g., Arne, E. S. J., Holmgren, A. The thirodoxin system in cancer. Sem. Cancer Biol. 16:420-426 (2006). In addition, Trx over-expression has also been associated with aggressive tumor growth. See, e.g., Id. This increase in expression level is likely related to changes in the Trx protein structure and function. For example, in pancreatic ductal carcinoma tissue, Trx levels were found to be elevated in 24 of 32 cases, as compared to normal pancreatic tissue; whereas glutaredoxin levels were increased in 29 of 32 of the cases. See, e.g., Nakamura, H., et al. Expression of thioredoxin and glutaredoxin, redox-regulating proteins, in pancreatic cancer. Cancer Detect. Prev. 24:53-60 (2000). Similarly, tissue samples of primary colorectal cancer or lymph node metastases had significantly higher Trx-1 levels than normal colonic mucosa or colorectal adenomatous polyps. See, e.g., Raffel, J., et al. Increased expression of thioredoxin-1 in human colorectal cancer is associated with decreased patient survival. J. Lab. Clin. Med. 142:46-51 (2003).

In two recent studies, Trx expression was associated with aggressive tumor growth and poorer prognosis. In a study of 102 primary non-small cell lung carcinomas, tumor cell Trx expression was measured by immunohistochemistry of formalin-fixed, paraffin-embedded tissue specimens. See, e.g., Kakolyris, S., et al. Thioredoxin expression is associated with lymph node status and prognosis in early operable non-small cell lung cancer. Clin. Cancer Res. 7:3087-3091 (2001). The absence of Trx expression was significantly associated with lymph node-negative status (P=0.004) and better outcomes (P<0.05) and was found to be independent of tumor stage, grade, or histology. The investigators also concluded that these results were consistent with the proposed role of Trx as a growth promoter in some human cancers, and overexpression may be indicative of a more aggressive tumor phenotype (hence the association of Trx overexpression with nodal positivity and poorer outcomes). In another study of 37 patients with colorectal cancer, Trx-1 expression tended to increase with higher Dukes stage (P=0.077) and was significantly correlated with reduced survival (P=0.004). After adjusting for Dukes stage, Trx-1 levels remained a significant prognostic factor associated with survival (P=0.012). See, e.g., Raffel, J., et al. Increased expression of thioredoxin-1 in human colorectal cancer is associated with decreased patient survival. J. Lab. Clin. Med. 142:46-51 (2003). It should be noted that GSH levels were not determined in either of the aforementioned studies.

The relationship between TrxR activity and tumor growth is less clear. Tumor cells may not need to increase expression of the TrxR enzyme, although its catalytic activity may be increased functionally. For example, human colorectal tumors were found to have 2-times higher TrxR activity than normal colonic mucosa. See, e.g., Mustacich, D. and Powis, G., Thioredoxin reductase. Biochem. J. 346:1-8 (2000). TrxR has also been reported to be elevated in human primary melanoma and to show a correlation with invasiveness. See, e.g., Schallreuter, K. U., et al. Thioredoxin reductase levels are elevated in human primary melanoma cells. Int. J. Cancer 48:15-19 (1991). Further evaluations relating TrxR enzyme levels and catalytic activity with cancer stage and outcome are required to fully elucidate this relationship.

### The Thioredoxin (Trx)/Thioredoxin Reductase (TrxR) System in Cancer Drug Resistance

As previously discussed, mammalian thioredoxin reductase (TrxR) is involved in a number of important cellular processes including, but not limited to: cell proliferation, antioxidant defense, and redox signaling. Together with glutathione reductase (GR), it is also the main enzyme providing reducing equivalents to many cellular processes. GR and TrxR are flavoproteins of the same enzyme family, but only the latter is a selenoprotein. With the catalytic site containing selenocysteine, TrxR may catalyze reduction of a wide range of substrates, but it can also be easily targeted by electrophilic compounds due to the extraordinarily high reactivity of the selenocysteine moiety. In a recent studies, the inhibition of TrxR and GR by anti-cancer alkylating agents and platinum-containing compounds was compared to the inhibition of GR. See, e.g., Wang, X., et al. Thioredoxin reductase inactivation as a pivotal mechanism of ifosfamide in cancer therapy. Eur. J. Pharmacol. 579:66-75 (2008); Wang, X., et al. Cyclophosphamide as a potent inhibitor of tumor thioredoxin reductase in vivo. Toxicol. Appl. Pharmacol. 218:88-95 (2007); Witte, A-B., et al. Inhibition of thioredoxin reductase but not of glutathione reductase by the major classes of alkylating and platinum-containing anticancer compounds. Free Rad. Biol. Med. 39:696-703 (2005). These studies found that: (i) the nitrosourea, carmustine, can inhibit both GR and Trx; (ii) the nitrogen mustards (cyclophosphamide, chlorambucil, and melphalan) and the alkyl sulfonate (busulfan) irreversibly inhibited TrxR in a concentration- and time-dependent manner, but not GR; (iii) the oxazaphosphorine, ifosfamide, inhibited TrxR; (iv) the anthracyclines (daunorubicin and doxorubicin) were not inhibitors of TrxR; (v) cisplatin, its monohydrated complex, oxaliplatin, and transplatin irreversibly inhibited TrxR, but not GR; and (vi) carboplatin could not inhibit either TrxR or GR. Other studies have shown that the irreversible inhibition of TrxR by quinones, nitrosoureas, and 13-cis-retinoic acid is markedly similar to the inhibition of TrxR by cisplatin, oxaliplatin, and transplatin. See, e.g., Amer, E. S. J., et al. Analysis of the inhibition of mammalian thioredoxin, thioredoxin reductase, and glutaredoxin by cis-diamminedichloroplatinum (II) and its major metabolite, the glutathione-platinum complex. Free Rad. Biol. Med. 31:1170-1178 (2001).

Studies have also shown that the highly accessible selenenylsulfide/selenolthiol motif of the Trx enzyme can be rapidly derivatized by a number of electrophilic compounds. See, e.g., Beeker, K, et al. Thioredoxin reductase as a pathophysiological factor and drug target. Eur. J. Biochem. 262:6118-6125 (2000). These compounds include, but are not limited to: (i) cisplatin and its glutathione adduct (see, e.g., Amer, E. S. J., et al. Analysis of the inhibition of mammalian thioredoxin, thioredoxin reductase; glutaredoxin by cis-diamminedichlamplatinum (II) and its major metabolite, the glutathioneplatinum complex. Free Rad. Biol. Med. 31:1170-1178 (2001)); (ii) dinitrohalobenzenes (see, e.g., Nordberg, J., et al. Mammalian thioredoxin reductase is irreversibly inhibited by dinitrohalobenzenes by alkylation of both the redox active selenocysteine and its neighboring cysteine residue. J. Biol. Chem. 273:10835-10842 (1998)); (iii) gold compounds (see, e.g., Gromer, S., et al. Human placenta thioredoxin reductase: Isolation of the selenoenzyme, steady state kinetics, inhibition by therapeutic gold compounds. J. Biol. Chem. 273:20096-20101 (1998)); (iv) organochalogenides (see, e.g., Engman, L., et al. Water-soluble organatellurium compounds inhibit thioredoxin reductase and the growth of human cancer cells. Anticancer Drug. Des. 15:323-330 (2000)); (v) different naphthazarin derivatives (see, e.g., Dessolin, I., et al. Bromination studies of the 2.3-dimethylnaphthazarin core allowing easy access to naphthazarin derivatives. J. Org. Chem. 66:5616-5619(2001)); (vi) certain nitrosoureas (see, e.g., Sehallreuter, K. U., et al. The mechanism of action of the nitrosourea anti-tumor drugs and thioredoxin reductase, glutathione reductase and ribonucleotide reductase. Biochim. Biophys. Acta 1054:14-20 (1990)); and (vii) general thiol or selenol alkylating agents such as C-vinylpyridine, iodoacetamide or iodoacetic acid (see, e.g., Nordberg, J., et al. Mammalian thioredoxin reductase is irreversibly inhibited by dinitrohalobenzenes by alkylation of both the redox active selenocysteine and its neighboring cysteine residue. J. Biol. Chem. 273:10835-10842 (1998)).

Similarly, several lines of evidence suggest that thioredoxin (Trx) may also be necessary, but is not sufficient in toto, for conferring resistance to many chemotherapeutic drugs. This evidence includes, but is not limited to: (i) the resistance of adult T-cell leukemia cell lines to doxorubicin and ovarian cancer cell lines to cisplatin has been associated with increased intracellular Trx-1 levels; (ii) hepatocellular carcinoma cells with increased Trx-1 levels were less sensitive cisplatin (but not less sensitive to doxorubicin or mitomycin C); (iii) Trx-1 mRNA and protein levels were increased by 4- to 6-fold in bladder and prostate cancer cells made resistant to cisplatin, but lowering Trx-1 levels with an antisense plasmid restored sensitivity to cisplatin and increased sensitivity to several other cytotoxic drugs; (iv) Trx-1 levels were elevated in cisplatin-resistant gastric and colon cancer cells; and (v) stable transfection of fibrosarcoma cells with Trx-1 resulted in increased cisplatin resistance. See, e.g., Biaglow, J. E. and Miller, R. A., The thioredoxin reductase/thioredoxin system. Cancer Biol. Ther. 4:6-13 (2005).

Glutathione may also play a role in anti-cancer drug resistance. Glutathione-S-transferases catalyze the conjugation of glutathione to many electrophilic compounds, and can be upregulated by a variety of cancer drugs. Glutathione-S-transferases possess selenium-independent peroxidase activity. Mn also has been shown to possess glutaredoxin activity. Some agents are substrates for glutathione-S-transferase and are directly inactivated by glutathione conjugation, thus leading to resistance. Examples of enzyme substrates include melphalan, carmustine (BCNU), and nitrogen mustard. In a panel of cancer cell lines, glutathione-S-transferase expression was correlated inversely with sensitivity to alkylating agents. Other drugs that upregulate glutathione-S-transferase may become resistant, because the enzyme also inhibits the MAP kinase pathway. These agents require a functional MAP kinase, specifically JNK and p38 activity, to induce an apoptotic response. See, e.g., Townsend, D. M. and Tew, K. D., The role of glutathione-S-transferase in anti-cancer drug resistance. Oncogene 22:7369-7375 (2003).

### The Use of Thioredoxin (Trx) Therapy in Cancer Patients

Since Trx shows anti-inflammatory effect in circulation, the clinical application of Trx therapy is now planned, especially because Trx has been shown to block neutrophil infiltration into the inflammatory site. For example, the administration of recombinant human Trx (rhTrx) inhibits bleomycin or inflammatory cytokine-induced interstitial pneumonia. See, e.g., Hoshino, T., et al. Redox-active protein thioredoxin prevents proinflammatory cytokine- or bleomycin-induced lung injury. Am. J. Respir. Crit. Care Med. 168:1075-1083 (2003). Therefore, acute respiratory distress syndrome (ARDS)/acute lung injury (ALI) is one disorder which is a good target for Trx therapy. ARDS/ALI is caused by various etiologies including anti-cancer agents such as gefitinib, a molecular-targeted agent that inhibits epidermal growth factor receptor (EGFR) tyrosine kinase. The safety of Trx therapy in cancer patients in currently being examined. Although the intracellular expression of Trx in cancer tissues is associated with, e.g., resistance to anti-cancer agents (see, e.g., Yokomizo, A., et al. Cellular levels of thioredoxin associated with drug sensitivity to cisplatin, mitomycin C, deoxrubicin, and etoposide. Cancer Res. 55:4293-4296 (1995); Sasada, T., et al. Redox control and resistance to cis-diamminedichloroplatinum (II) (CDDP); protective effect of human thioredoxin against CDDP-induced cytotoxicity. J. Clin. Investig. 97:2268-2276 (1996)), there is no evidence showing that exogenously administered rhTrx promotes the growth of cancer. For example, there is no promoting effect of administered rhTrx on the growth of the tumor planted in nude mice. In addition, administered rhTrx has no inhibitory effect on the anti-cancer agent to suppress the tumor growth in nude mice. It may be explained by that the cellular uptake of exogenous Trx is quite limited and administered Trx in plasma immediately becomes the oxidized form which has no tumor growth stimulatory activity as previously mentioned.

### Specific Examples and Experimental Results for Thioredoxin (Trx)

Studies in the specific example of Trx described herein demonstrate that Tavocept and Tavocept-derived mesna disulfide heteroconjugates act as alternative substrates for the Trx/TrxR coupled system. As alternative substrates, Tavocept and Tavocept-derived Tavocept-derived heteroconjugates can compete with endogenous substrates, like insulin, for turnover and, thereby, inhibit turnover of the endogenous substrates. It is hypothesized that Tavocept and Tavocept-derived heteroconjugates may increase patient survival by the direct inhibition of thioredoxin in cancers that overexpress thioredoxin or have increased thioredoxin activity, including adenocarcinoma of the lung. Additionally, it is also hypothesized that Tavocept might covalently modify cysteines in Trx (i.e., Tavocept might xenobiotically modify cysteine residues on Trx), thus yielding a Trx-mesna species that is functionally distinct from apo-Trx (i.e., apo-Trx is Trx that contains no mesna modification) and providing an additional mechanism for modulation of Trx activity. Also disclosed herein are results from the first reported experimental studies (LC-MS and X-ray crystallography) elucidating stable covalent modification of thioredoxin (Trx) in vitro by a small molecule disulfide, Tavocept. The effect of Tavocept and Tavocept-derived mesna-disulfide heteroconjugates on the thioredoxin system may help explain Tavocept-mediated antitumor potentiation and survival benefits seen in clinical trials.

### Native and IEF PAGE Analysis

A. Materials

Thioredoxin (Trx, human), glutathione, and NADPH were purchased from Sigma. Bovine and rat Trx reductase (TrxR) were obtained from American Diagnostica and Sigma, respectively. It should be noted that individual comparisons of the rat and bovine TrxR sequences to that of human TrxR reveal that there is a >90% sequence identity with the human protein.

Tris-glycine native gels, Tris-glycine SDS gels and IEF PAGE gels (pI 3.5-8.0), their associated running buffers, loading buffers, and protein standards were purchased from Invitrogen. Gel Code Blue Stain reagent was purchased from Thermo Scientific. All other reagents were obtained from Sigma-Aldrich or BioRad.

B. Methods

Sample Preparation for PAGE Analysis of Thioredoxin Incubated with and without Tavocept

Recombinant human Trx (1.5 mg, SigmaAldrich) was reduced using a vast excess of DTT (50 mM) in Tris buffer (100 mM, pH 8.0) at 37° C. for 50 minutes. DTT was removed using a G25 Sephadex column (GE Life Sciences) and the DTT-free, reduced protein was incubated with either Tavocept (10 mM) or buffer alone at 37° C. (final reactions volumes were approximately 160 μL). At time selected times (0-48 hours), aliquots were removed and subjected to TrisGlycine Native or TrisGlycine SDS PAGE analysis. Gels were fixed in acetic acid/methanol and stained with Coomassie R-250.

Additionally, samples of apo-Trx, Trx-mesna, and Trx-GSH, taken from 0, 2, 4 and 6 hour time-points that were purified away from excess Tavocept and glutathione disulfide, were analyzed using IEF PAGE (Trx-GSH IEF data not shown). For some of these IEF PAGE experiments, samples were divided into two aliquots; wherein one aliquot was treated with Trx reductase and NADPH for 30 minutes prior to loading on the IEF gel and the other aliquot was not. IEF gels were fixed in 20% trichloroacetic acid and stained with Gel Code Blue Stain reagent (Thermo Scientific).

### PAGE Analysis of Thioredoxin Incubated with and without Tavocept

Recombinant human Trx (1.5 mg) was reduced using a vast excess of DTT (50 mM) in Tris buffer (100 mM, pH 8.0) at 37° C. for 50 minutes. DTT was removed using a G25 Sephadex column (GE Life Sciences) and the DTT-free, reduced protein was incubated with either Tavocept (10 mM) or buffer alone at 37° C. (final incubation reaction volumes were approximately 160 μL). At time selected times (0-48 hours), aliquots were removed and subjected to TrisGlycine Native or TrisGlycine SDS PAGE analysis. Gels were fixed in acetic acid/methanol and stained with Coomassie R-250.

Additionally, samples of apo-Trx, Trx-mesna and Trx-GSH, taken from 0, 2, 4, and 6 hour time-points that were purified away from excess Tavocept and glutathione disulfide, were analyzed using IEF PAGE (Trx-GSH IEF data not shown). For some of these IEF PAGE experiments, samples were divided into two aliquots; wherein one aliquot was treated with Trx reductase and NADPH for 30 minutes prior to loading on the IEF gel and the other aliquot was not. IEF gels were fixed in 20% trichloroacetic acid and stained with Gel Code Blue Stain reagent (Thermo Scientific).

C. Results

### Native PAGE Results

### A DTT-Sensitive Tavocept-Derived Modification Occurs on Trx

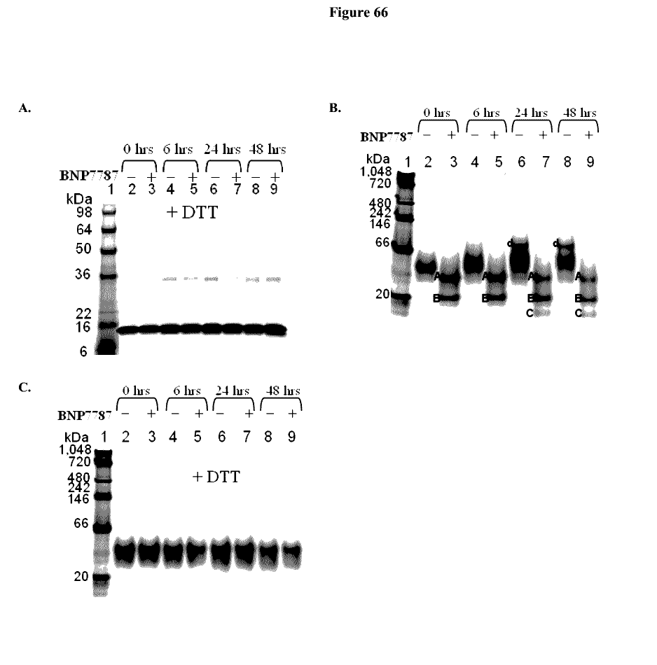

Under denaturing, reducing conditions, recombinant human Trx migrates predominantly as a single band with a molecular weight of 12 kDa (see, FIG. 66, Panel A). Incubation of Trx with Tavocept resulted in an altered Tris-glycine native gel electrophoresis profile, consistent with the idea that a Tavocept-derived mesna moiety forms a mixed disulfide on Trx (see, FIG. 66, Panel B). When this putative Trx-mesna species was treated with DTT, it was reduce to a species that co-migrated with apo-Trx (FIG. 66, Panel C) on the Tris-glycine native gel. In the native gels shown in FIG. 66, Panels B and C, unmodified Trx migrates consistent with a higher order oligomer (FIG. 66, Panel B, lane 2).

### Native PAGE Detects Trx Species with Altered Electrophoretic Mobility in Trx Samples Incubated with Tavocept

Native PAGE indicates that apo-Trx migrates with an apparent mobility that is distinct from Trx that has been incubated with Tavocept (see, FIG. 66, Panel B). Two or more novel, distinct Trx species appear in samples incubated with Tavocept for 6 hours or less (see, FIG. 66, Panel B, lanes 3 and 5) and are indicated in FIG. 66, Panel B as “A” and “B”. A third species, indicated as “C”, appears in samples where Trx has been incubated with Tavocept for 24 hours or more (see, FIG. 66, Panel B, lanes 7 and 9). These A, B, and C species represent Trx species containing a mixed disulfide between one or more of the five Trx cysteine residues (i.e., Cys32, Cys35, Cys62, Cys69, or Cys73) and a Tavocept-derived mesna moiety. Additionally, as shown in FIG. 66, Panel B over time a second species appears in the apo-Trx samples at 24 and 48 hours (see, lanes 6 and 8, labeled as “D”) and is probably a Trx oligomer that forms as the 5 reactive cysteines residues oxidize.

### IEF PAGE Results

### Tavocept Modifies Trx Forming Species which can be Reduced Back to Apo-Trx by Trx Reductase and NADPH

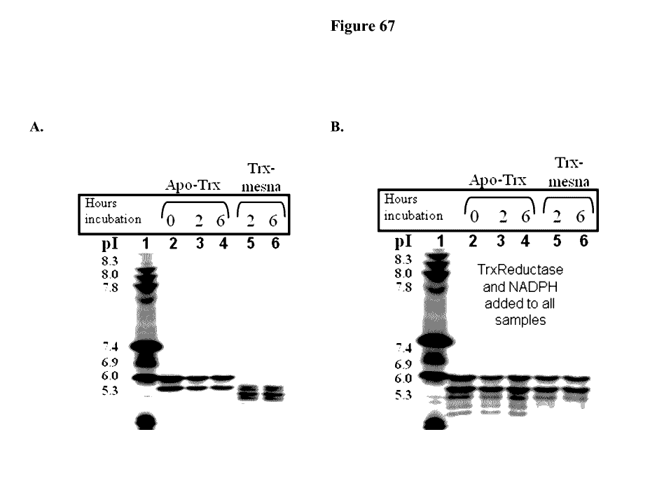

IEF gel analysis (see, FIG. 67, Panel A) indicated that apo-Trx (purchased from Sigma) migrated as two bands. This phenomenon is common among recombinant human proteins expressed in E. coli and given that the Trx from Sigma migrates as one predominant species under DTT/SDS-PAGE conditions (see, FIG. 67, Panel A), it is thought that the two bands seen in apo-Trx samples on IEF gel (see, FIG. 67, Panel A) correspond to two slightly different conformational versions of the protein that have slightly different pis. The data indicate that Tavocept results in a shift in Trx migration due to modification of Trx by Tavocept forming a Trx-mesna species (see, FIG. 67, Panel A, lanes 5 and 6). Incubation of Trx-mesna samples with Trx reductase and NADPH prior to loading on the IEF gel results in reduction of the Trx-mesna species back to apo-Trx (see, FIG. 67, Panel B, lanes 5 and 6; note that the control apo-Trx samples on this gel were also incubated with Trx reductase and NADPH prior to loading). The appearance of such multiple bands, after incubation with Trx reductase and NADPH in samples originally from the apo-Trx and Trx-mesna reactions, most likely reflects variations on overall 3- and/or 4-fold in the apo-Trx—perhaps due to the 5 reactive cysteine residues that are present in the Trx molecule.

### Mass Spectroscopy Analysis of Tavocept-Derived Mesna Adducts on Cys62/Cys69 and Cys73 on Human Trx

A. Materials

Thioredoxin (Trx, human) was purchased from Sigma. Tavocept was prepared by a proprietary method (>97% purity, no mesna was detected by mass spectroscopy). PD spin traps, NAP5, and NAP10 columns were purchased from GE Healthcare. Symmetry C18 HPLC column was purchased from Waters (Franklin, Mass.). All other reagents were obtained from Sigma-Aldrich or VWR. Trypsin Gold and glutamylendopeptidase were purchased from Promega and BioCol GMBH, respectively.

B. Methods

### Incubation of Thioredoxin with Tavocept, Mesna, or Glutathione Disulfide for MS Analyses

Recombinant human Trx (2.5 mg, 0.208 μmoles) was reduced using a vast excess of DTT (12.5 μmoles) in Tris buffer (100 mM, pH 8.0, 300 μL total volume) at 37° C. for 50 minutes. DTT was removed using a G25 Sephadex column (GE Life Sciences) and the DTT-free, reduced protein was incubated with either Tavocept (10 mM), mesna (10 mM), glutathione disulfide (10 mM), or buffer alone at 37° C. (reactions were either 500 μL or 1 mL final volume). After 4 to 6 hours, all reactions were chromatographed over a G25 Sephadex column to remove the residual (unreacted) small molecules (the buffer control was also chromatographed to insure identical handling of all samples).

### Protease Digests on Thioredoxin Incubations

After gel filtration removal of the excess, unreacted Tavocept, mesna, or glutathione disulfide, the Trx protein was digested in preparation for Mass Spectroscopy analyses. Briefly, the G25 chromatographed Trx incubation reactions were digested with Trypsin Gold (9 μg per reaction) for 12 hours at 37° C. In some cases, after the Trypsin digest, glutamylendopeptidase (BioCol, 10 μg) and CaCl₂ (2 mM final concentration) were added and the reaction was allowed to incubate at room temperature for an additional 8-12 hours. Trypsin- and Trypsin/glutamylendopeptidase-digested samples were then analyzed using LC MS.

### Mass Spectroscopy Analyses of Trypsin Digested Trx Modified by Tavocept

A Symmetry C18 HPLC column (Waters, Franklin, Mass.; 3.5 μm; 4.6×75 mm) and a Waters Alliance liquid chromatography system (Waters 2695, Franklin, Mass., USA) coupled to a Micromass single quadropole mass detector (Micromass ZMD, Manchester, UK) were used to analyze fragments from Trypsin and/or glutamyl endopeptidase digested human Trx. The mobile phase contained 0.1% of formic acid throughout the run and the flow rate was 0.35 mL/min. The elution scheme involved the following steps: Step 1—0 to 3.5 minutes mobile phase was 95% water/5% acetonitrile; Step 2—3.5 to 20 minutes linear gradient to 10% water/90% acetonitrile; Step 3—20-30 minutes hold at 10% water/90% acetonitrile; Step 4—30-40 minutes linear gradient from 10% water/90% acetonitrile to 95% water; 5% acetonitrile. Positive-ion and negative-ion ionization modes across the mass ranges of 500-3000 Da (positive-ion mode) and 100-1700 Da (negative-ion mode) were used.

### Mass Spectroscopy Results

### Mass Spectroscopy Identification of Tavocept-Derived Mesna Adducts on Cysteine 62/69 and Cysteine 73 Containing Trx Fragments

Several groups have reported that modification of cysteine residues in Trx result in inactivation of Trx or impair Trx activity or functioning. See, e.g., Han, S., Force field parameters for S-nitrosocysteine and molecular dynamics simulations of S-nitrosated thioredoxin. Biochem. Biophys. Res. Comm. 377:612-616 (2008); Kirkpatrick D L, Kuperus M, Dowdeswell M, et al., Mechanisms of inhibition of the thioredoxin growth factor system by antitumor 2-imidazolyl disulfides. Biochem. Pharmacol. 55:987-994 (1998); Casagrande S, Bonetto V, Fratelli M, et al., Glutathionylation of human thioredoxin: a possible crosstalk between the glutathione and thioredoxin systems. Proc. Natl. Acad. Sci. U.S.A. 99:9745-9749 (2008).

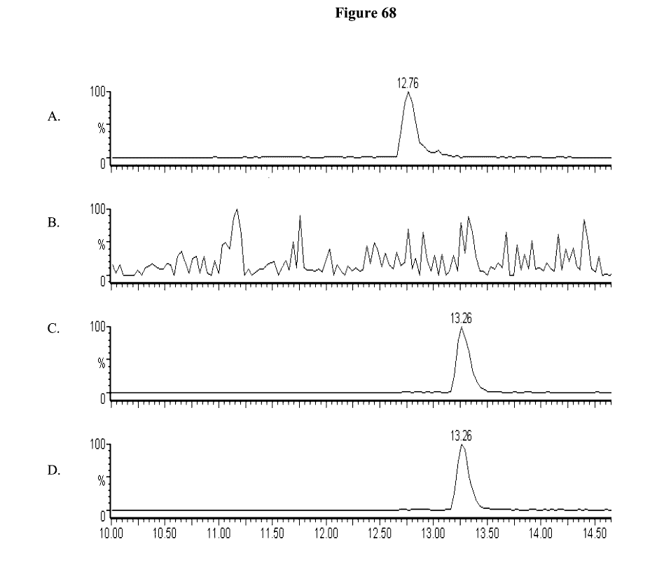

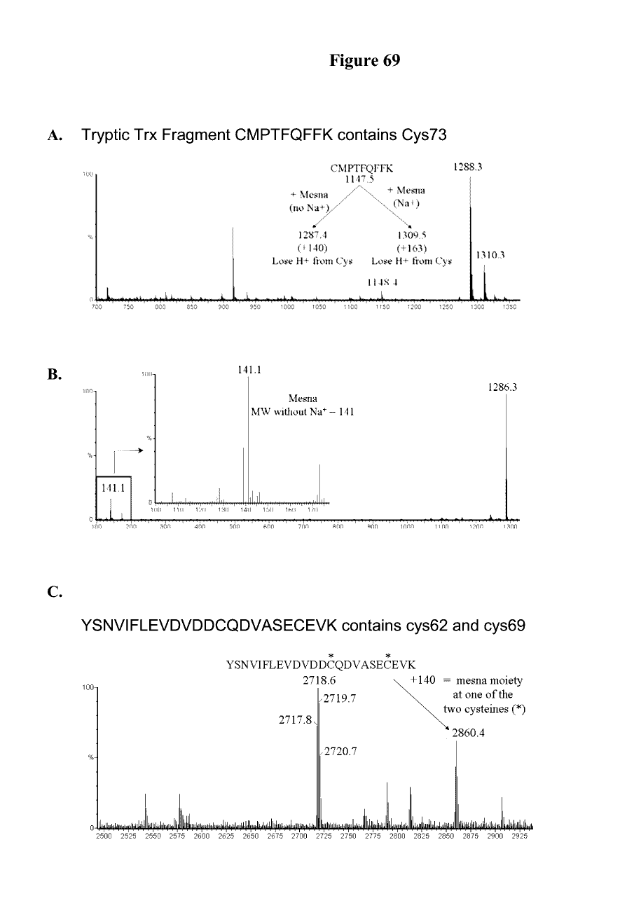

For Mass Spectroscopy studies, purified recombinant human Trx was incubated for 4 to 6 hours with either Tavocept, mesna, glutathione, or glutathione disulfide. Unreacted (free) Tavocept, mesna, glutathione, and glutathione disulfide were removed using size exclusion chromatography leaving only the protein Trx (or Trx with adducts of glutathione or mesna). Trx was then digested using either Trypsin alone or Trypsin in combination with glutamyl endopeptidase and analyzed by liquid chromatography mass spectroscopy (LC MS) for the presence of mesna or glutathione adducts. In control reactions with glutathione, it was observed that Trx was glutathionylated at cysteine-73 (Cys73). Additionally, liquid chromatographic analysis revealed a new peak in the reactions incubated with Tavocept or mesna (see, FIG. 68, Panels C and D). Positive- and negative-ion mass spectroscopy analyses of these new peaks revealed the presence of a mesna adduct on Cys73 of Trx (see, Table 21, Row 8: CMPTFQFFK Trx fragment; see also, FIG. 69, Panels A and B). Additionally, the results showed the formation of one additional mesna adduct on the Trx 24-residue fragment containing cysteine-62 (Cys62) and cysteine-69 (Cys69) (see, Table 21, Row 7: YSNVIFLEVDVDDCQDVASECEVK Trx fragment; see also, FIG. 69, Panel C); however, attempts to identify which cysteine in this 24 residue-fragment contained the Tavocept-derived mesna adduct (i.e., Cys62 or Cys69) were unsuccessful. This was most likely due to the requirement of the additional digestion step with glutamyl endopeptidase that did not proceed with high efficiency cleavage.

TABLE 21
Summary of fragments generated from Trypsin digest of His-tagged,
recombinant human Thioredoxin

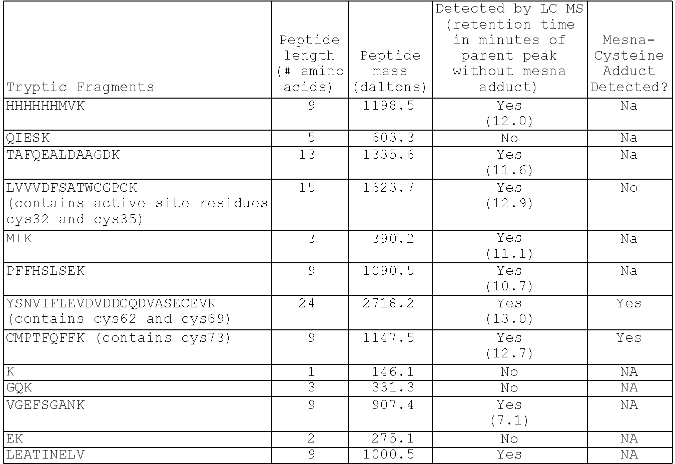

(NA = not applicable; retention times in minutes are shown in parentheses)

### Enzyme Activity Assays

A. Materials

L-Cystine, DL-homocysteine, L-homocystine, glutathione (GSH), glutathione disulfide, tetrabutylammonium dihydrogen phosphate were purchased from Sigma (St. Louis, Mo.); L-Cysteine was purchased from Aldrich (Milwaukee, Wis.); HPLC grade water and acetonitrile were obtained from Burdick & Jackson (VWR). Thioredoxin (Trx, human), glutathione, and NADPH were purchased from Sigma. Bovine and rat Trx reductase (TrxR) were obtained from American Diagnostica and Sigma, respectively (individual comparisons of the rat and bovine TrxR sequences to that of human TrxR reveal a >90% sequence identity to the human protein). Cysteinylglycine and γ-glutamylcysteine were purchased from Bachem. All other reagents were obtained from Sigma-Aldrich or BioRad. PD spin traps, NAP5, and NAP10 columns were purchased from GE Healthcare.

B. Methods

### Synthesis of Tavocept and Tavocept-Derived Mesna-Disulfide Heteroconjugates

Tavocept was prepared by a proprietary method (purity>97%, no mesna was detected by mass spectroscopy). Mesna was purchased from Sigma (purity≧98%). The heteroconjugates of mesna described herein were prepared by a solid-state synthesis method. See, e.g., Shanmugarajah D, Ding D, Huang Q, Chen X, Kochat H, Petluru P N, Ayala P Y, Parker A R, Hausheer F H. Analysis of Tavocept thiol-disulfide exchange reactions in phosphate buffer and human plasma using microscale electrochemical high performance liquid chromatography. J. Chromatogr. B. Analyt. Technol. Biomed. Life Sci. 877:857-866 (2009).

In brief, sodium-2-mercaptoethanesulfonate (mesna) was bound to the sulfinated polystyrene resin through a thiolsulfonic bond, then reacted with commercially available thiol-containing compounds (mesna, glutathione, cysteine, cysteinylglycine, γ-glutamylcysteine, and homocysteine) in aqueous solutions to give high purity mesna-disulfide heterconjugates: mesna-glutathione disulfide (MSSG; 100%); mesna-cysteine disulfide (MSSC; 98.6%); mesna-cysteinyl glycine disulfide (MSSCG; 100%); mesna-cysteinyl glutamate disulfide (MSSCE; 98.3%); and mesna-homocysteine disulfide (MSSH; 100%).

### Thioredoxin and Thioredoxin Reductase NADPH Oxidation and Insulin Precipitation Assay

The activities of TrxR and Trx, with Tavocept, MSSC, MSSG, MSSCG, MSSCE, or MSSH as potential alternative substrates, were determined by monitoring NADPH oxidation at 340 nm according to the Holmgren method. See, Luthman M and Holmgren A. Rat liver thioredoxin and thioredoxin reductase: purification and characterization. Biochemistry 21:6628-6633 (1982). A typical assay mixture contained buffer (50 mM potassium phosphate, pH 7.0, 1 mM EDTA), NADPH (200 μM), and bovine TrxR (1.6 μg, 0.138 μM). Assays were run with and without recombinant human Trx (4.8 μM). Positive controls used insulin (86 μM) as the substrate; negative controls did not contain a disulfide substrate. The ability of Tavocept, or one of the mesna-disulfide heteroconjugates, to serve as alternative substrates, facilitating NADPH oxidation in the absence of insulin, was evaluated (see, Table 22). All disulfides were added to reactions as 10× solutions in buffer. The total volume of each reaction was 0.1 mL. Reactions were initiated by the addition of TrxR and were incubated at 25° C. for 40 minutes. Reactions were analyzed using a Molecular Devices SpectraMax Plus UV/vis plate reader and the activity was calculated using a 4 minute linear portion of each assay.

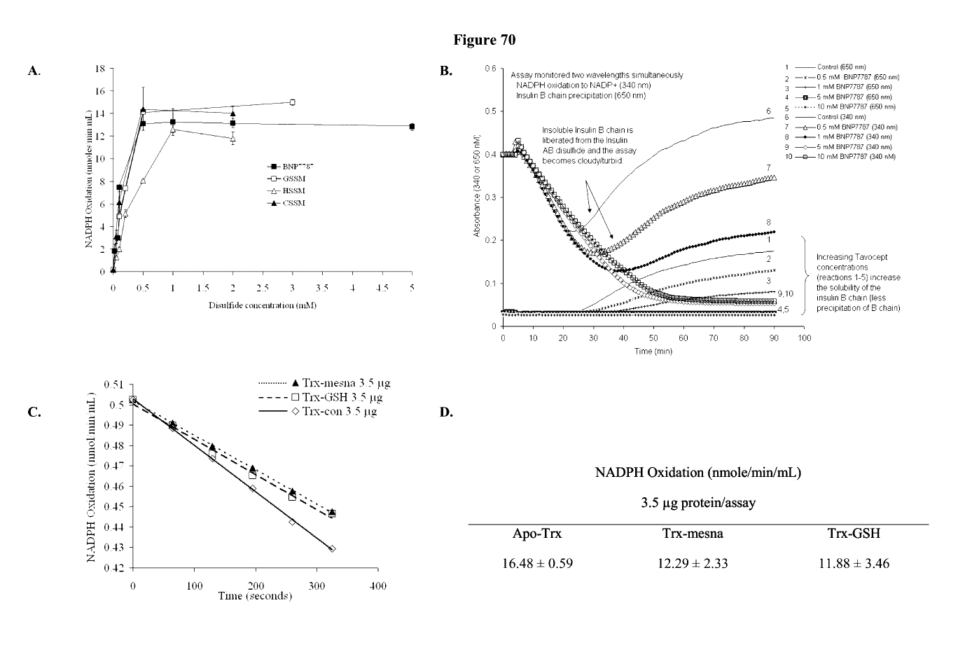

The effects of Tavocept (0-10 mM) on the TrxR/Trx catalyzed reduction of the insulin disulfide were also monitored using a dual wavelength assay that followed both NADPH oxidation at 340 nm and the precipitation of the insulin B chain at 650 nm. A typical assay mixture contained buffer (50 mM potassium phosphate, pH 7.0, 1 mM EDTA), NADPH (200 μM), rat liver TrxR (0.1 μM), human Trx (4.8 μM), and varying Tavocept concentrations. Reactions were initiated by adding insulin (86 μM). The total volume of each reaction was 0.2 mL. Reactions were analyzed for up to 80 minutes using a Molecular Devices SpectraMax Plus UV/vis plate reader observing at 650 nm (see, FIG. 70, Panel B). Trx cleaves the insulin AB chain disulfide resulting in the liberation of the insoluble free thiol form of the B chain.

Additionally, apo-Trx, Trx-mesna and Trx-GSH species were prepared and excess/unreacted Tavocept or glutathione disulfide were removed. Briefly, recombinant human Trx (1.5 mg) was reduced using a vast excess of DTT (50 mM) in Tris buffer (100 mM, pH 8.0) at 37° C. for 50 minutes. DTT was removed using a G25 Sephadex column (GE Healthcare) and the DTT-free, reduced protein was incubated with either Tavocept (10 mM), glutathione disulfide (10 mM), or buffer alone at 37° C. (the final volume of the incubation reactions were approximately 330 μL). At 0, 2, 4, and 6 hours incubation, aliquots were removed and unreacted Tavocept or glutathione was removed using a PD Spin Trap (GE Healthcare). The samples were then assayed for protein content (Bradford assay, BioRad), and then evaluated in the TrxR/Trx insulin disulfide reduction assay. See, Luthman M and Holmgren A. Rat liver thioredoxin and thioredoxin reductase: purification and characterization. Biochemistry 21:6628-6633 (1982).

### Enzyme Activity Assay Results

### Tavocept and Tavocept-Derived Mesna-Disulfide Heteroconjugates are an Alternative Substrate for Thioredoxin (Trx) and Function as Competitive Inhibitors

Although Trx exhibits a preference for insulin and other proteins as substrates, it was hypothesized that Trx might catalyze the reduction of the disulfide bond in Tavocept and/or Tavocept-derived mesna-disulfide heteroconjugates and that these interactions between Tavocept and Trx might be correlated to survival benefits in patients with, e.g., non-small cell lung cancer.

Tavocept and all of the Tavocept-derived mesna disulfide-heteroconjugates that were tested were readily reduced by Trx in the presence of TrxR and NADPH (Table 22, Reaction B). In contrast, TrxR alone did not detectably reduce Tavocept or the mesna-disulfide heteroconjugates (Table 22, Reaction A). Tavocept and Tavocept-derived mesna-disulfide heteroconjugates probably lack structural functionalities and/or structural “bulk” needed to serve as effective inhibitors or alternative substrates for TrxR in the absence of Trx and insulin despite the fact that TrxR can accept a broad range of substrates. See, e.g., Becker K, Gromer S, Schirmer R H, Muller S. Thioredoxin Reductase as a Pathophysiological Factor and Drug Target. Eur. J. Biochem. 267:6118-6125 (2001); Mustacich D and Powis G. Thioredoxin reductase. Biochem. J. 346 Pt 1:1-8 (2000). However, it was noted what appeared to be an effect on the TrxR/Trx mediated rate of NADPH oxidation during assays monitoring both NADPH oxidation and insulin B chain precipitation (see, FIG. 70, Panel B) in the presence of Tavocept consistent with Tavocept acting as an alternative substrate inhibitor or otherwise modulating TrxR/Trx activity and this effect was evaluated further (see, infra).

The Tavocept-derived mesna-disulfide heteroconjugates mesna-glutathione (MSSG), mesna-cysteine (MSSC) and mesna-cysteinylglycine (MSSCG) were preferred slightly by Trx over mesna-homocysteine (MSSH) and mesna-cysteinylglutamate (MSSCE), although all of the Tavocept-derived mesna-disulfide heteroconjugates were reasonably good substrates for Trx. By way of non-limiting example, compare NADPH oxidation values for Reaction A in Table 22 (with TrxR alone) to NADPH oxidation values for Reaction B in Table 22 (with TrxR in combination with Trx). Cumulatively, the disclosed data indicated that Tavocept and Tavocept-derived mesna-disulfide heteroconjugates act as alternative substrates of Trx with the potential to compete with and inhibit the reduction of endogenous Trx substrates (i.e., they are alternative substrate inhibitors of Trx).

TABLE 22
Tavocept and Tavocept-Derived Mesna-Disulfide Heteroconjugates are
Alternative Substrates for Thioredoxin (Trx)
Coupled to Thioredoxin Reductase (TrxR)

| | NADPH Oxidation (nmoles/min/mL)^{a,b} | |
|---|---|---|
| | Reaction A^{c} | Reaction B |
| | X—SS—Y + NADPH + | X—SS—Y + NADPH + |
| | 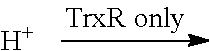 | 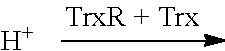 |
| | NADP⁺ + XSH + YSH | NADP⁺ + XSH + YSH |
| No Disulfide | 0.14 ± 0.01 | 0.30 ± 0.04 |
| Insulin | 0.3 ± .03 | 18.0 ± 0.14 |
| Tavocept | 0.3 ± 0.01 | 13.1 ± 0.2 |
| (MSSG) | 0.3 ± 0.02 | 14.1 ± 0.1 |
| MSSC | 0.2 ± 0.03 | 14.4 ± 0.2 |
| MSSR | 0.0 ± 0.03 | 8.6 ± 0.06 |
| dMSSCE | 0.3 ± 0.02 | 9.6 ± 0.2 |
| dMSSCG | 0.2 ± 0.04 | 15.8 ± 0.3 |

^{a}Oxidation rates were calculated from the 4 minute change in absorbance and triplicate or more assays. Disulfide concentrations were 0.5 mM, except for insulin. Insulin concentration was 86 μM.
^{b}A two-way ANOVA analysis was performed on the whole dataset. The difference between reaction rates for type A reactions and type B reactions is statistically significant (p-value = 0.0001), and affected by the disulfide substrate used in the reaction (p-value = 0.0001).
^{c}Zero was assigned to the MSSH rate, Reaction A where there were either slight positive absorbance changes or absorbance changes of less than 0.0001.
^{d}The MSSCE and MSSCG compounds are mesna adducts of the cysteinyl-glutamate and cysteinyl-glycine intermediates in the biosynthesis and degradation of GSH, respectively.

### Tavocept-Derived Mesna Modification of Trx Impairs Activity and Results in Reduced Initial Velocity in Protein Assays

As discussed above, Tavocept serves as an alternative substrate for TrxR/Trx (see, Table 22, Reaction B) and, therefore, in assays where Tavocept is present but insulin is absent, NADPH oxidation still occurred (see, Table 22). Consequently to determine whether or not covalent modification of Trx by a Tavocept-derived mesna moiety resulted in a Trx species (i.e., Trx-mesna) that interfered with reduction of the insulin substrate in the TrxR/Trx coupled system relative to apo-Trx, Trx-mesna had to be purified away from excess, free Tavocept present in the reaction used to generate Trx-mesna. Similar work has been reported previously for Trx modified by glutathione. Trx was incubated with Tavocept or glutathione disulfide (glutathione disulfide was included as a control based on earlier results) for the times indicated and, subsequently, unreacted/free Tavocept or glutathione disulfide was removed using a gel filtration step. Respectively, this provided Trx-mesna and Trx-GSH species that did not contain residual, unreacted/free Tavocept or glutathione disulfide (Note: as a control, apo-Trx was subjected to the same manipulations). These isolated Trx-mesna and Trx-GSH were assayed in the Trx/TrxR insulin reduction assay and a clear effect on initial velocity, relative to apo-Trx, was observed (see, FIG. 70, Panels C and D). Similar to what was previously reported with Trx-GSH (see, e.g., Casagrande S, Bonetto V, Fratelli M, et al., Glutathionylation of human thioredoxin: a possible crosstalk between the glutathione and thioredoxin systems. Proc. Natl. Acad. Sci. U.S.A. 99:9745-9749 (2002), it was observed that excess Trx reductase reduced Trx-mesna back to apo-Trx (see, FIG. 67, Panel B). In vitro, excess Trx reductase was utilized in these assays relative to Trx and, therefore, exhibited fairly rapid conversion of Trx-mesna and Trx-GSH back to apo-Trx. Nevertheless, the inhibitory effect of Trx-mesna, relative to apo-Trx, on initial velocity was evident (see, FIG. 70, Panels C and D) and would be expected to translate to an in vivo effect as well.

### X-ray Crystallographic Studies on Trx Covalently Modified by a Tavocept-Derived Mesna Moiety

A. Materials

Tavocept was prepared by a proprietary method (purity >97%, no mesna was detected by Mass Spectroscopy). Oligonucleotide primers used in cloning and mutagenesis were purchased from EMD. Roche Complete Protease Inhibitor tablets were purchased from Roche and benzonase was purchased from SigmaAldrich or EMD. IPTG was purchase from SigmaAldrich. Wild type and mutant thioredoxin proteins were purified from a pET-15b expression system. A Ni²⁺ charged IMAC resin was purchased from BioRad. PEGION, CRYSTALS HT, and PEGRX were purchased from Hampton Research and JCSG and BASIC were purchased from JENA Biosciences. All other items were purchased from SigmaAldrich.

B. Methods

### Cloning and Site Directed Mutagenesis to Produce the E13K, D16K, E95K, E103K Thioredoxin Protein

Wild-Type thioredoxin was cloned into a proprietary vector containing an N-terminal 6×his tag cleavable by TEV protease. Wild-type DNA underwent three rounds of mutagenesis using the following primers: E13K/D16K: 5′-GCA AAA CCG CTT TTC AGA AAG CTC TGA AGG CAG CCG GTG ACA AAC-3′ and 5′-GTT TGT CAC CGG CTG CCT TCA GAG CTT TCT GAA AAG CGG TTT TGC-3′; E95K: 5′-TCT CCG GCG CAA ACA AAA AAA AAC TGG AAG CAA CC-3′ and 5′-GGT TGC TTC CAG TTT TTT TTT GTT TGC GCC GGA GA-3′; E103K: 5′-AAA AAC TGG AAG CAA CCA TCA ATA AAC TGG TGT GAC TCG-3′ and 5′-CGA GTC ACA CCA GTT TAT TGA TGG TTG CTT CCA GTT TTT-3′. The final cloning product, Trx containing E13K, D16K, E95K, and E103K mutations, was verified by DNA sequencing.

### Protein Expression and Purification

The E13K, D16K, E95K, and E103K Trx mutant was expressed in BL21(DE3) cells. Cells were grown at 37° C. to OD₆₀₀˜0.6. Protein expression was induced with 0.5 mM IPTG at 18° C. overnight. The cell biomass was harvested and stored at −80° C. until ready to use. Purification of target protein was done using a 3 column system. The cell biomass was lysed by sonication in Buffer A (50 mM Tris-HCl, pH 7.8, 500 mM NaCl, 10% glycerol, 20 mM imidazole, 20 mM βME) containing 1 Roche Complete Protease Inhibitor Tablet, and Benzonase (20,000 units). Target protein was purified using a Ni²⁺ charged IMAC resin and eluted with imidazole (250 mM, pH 7.0). Peak fractions were cleaved with 2 mg TEV protease overnight in Buffer A. Cleaved protein was chromatographed over a Ni²⁺ charged IMAC resin collecting the column eluate. Aggregated oligomeric protein was separated from monomeric protein using size exclusion in Tris buffer (50 mM, pH 7.5) containing NaCl (250 mM) and DTT (5 mM). Monomeric protein was concentrated to ˜60 mg/ml and additional DTT (50 mM) was added. In addition, the protein was warmed to 30° C. for 60 minutes to facilitate complete DTT-mediated reduction of the disulfides. Protein not used immediately was flash frozen in liquid N₂ and stored at −80° C. The final purified protein contained an N-terminal sequence of GAGT which is part of the TEV recognition site. The last residue (threonine) of the tag was ordered in the electron density map.

### Preparation and Whole Protein LC MS Analysis of Tavocept-Derived Mesna Adduct on Thioredoxin

Adduct was prepared as described above with some modifications designed to increase the likelihood of success in crystallization of the protein. In brief Trx (60 mg/mL) in Tris (20 mM, pH 7.5), NaCl (250 mM), and DTT (5 mM) was fully reduced by adding a vast excess of DTT (final concentration 50 mM). This reaction was incubated for 1 hour at 30° C. followed by overnight incubation at 4° C. Next, excess DTT was removed using ultrafiltration (possessing a 10 kDa MW cut-off) to exchange the protein 5-times against glycine (50 mM, pH 9.0)/NaCl (250 mM). This exchanged solution was supplemented with DTPA (1 mM), Neocuprione (1 mM), and Tavocept (40 mM) and then incubated at 4° C. overnight at either pH 9.0 or pH 7.0. Aliquots of this solution were analyzed by ESI LC-MS to confirm the presence of adduct(s) prior to initiation of crystallization experiments.

### Crystallization of a Tavocept-Derived Mesna Adduct on E13K, D16K, E95K, E103K Thioredoxin

Tavocept-derived mesna adduct(s) on Trx (E13K, D16K, E95K, E103K; hereinafter referred to as Trx) were formed at either pH 9.0 or pH 7.0 and, from these adduct formation reactions, crystals were grown at either pH 8.5 or 7.0 using the sitting drop, vapor diffusion methodology in a 96 well format (Greiner plates) at 60 or 160 mg/ml thioredoxin with 1 mM DTPA, 1 mM neocuprione, and 40 mM Tavocept at 20° C. These initial broad screens produced crystals under a wide range of conditions (data not shown) and the screens used included: (i) PEGION, CRYSTALS HT, and PEGRX (Hampton Research) and (ii) JCSG and BASIC (JENA Biosciences). Multiple rounds of optimization were completed and included fine screens, varying the protein concentration and varying the protein to reservoir ratios to obtain diffraction quality crystals. In the Trx pH 9.0/8.5 structure (adduct formed at pH 9.0, crystals grown at pH 8.5), the best crystals were obtained in 20% ethanol, 0.1 M Tris (at 60 mg/mL Trx protein) and diffracted to 2.5 Å (C2 space group). In Trx pH 9.0/7.0 (adduct formed at pH 9.0, crystals grown at pH 7.0), the best crystals were obtained in 20% PEG3350, 0.2 M KCl (60 mg/mL Trx protein) and diffracted to 2.8 Å (C2 space group). In Trx pH 7.0/7.0 (adduct formed at pH 7.0, crystals grown at pH 7.0), the best crystals were obtained in 28% PEG3350, 0.2M KCl (160 mg/mL Trx protein) and diffracted to 1.85 Å resolution (P2₁ space group).

### X-Ray Diffraction Data

Diffraction data were collected at a wavelength of 1.0 Å on a Rayonix 225 detector array at beamline LS-CAT 21 ID-F at the Advanced Photon Source (Argonne National Laboratory) or on an X detector at beamline X at the Advanced Light Source (Lawrence Berkeley National Laboratory). For the Trx pH 9.0/8.5 structure, a Tavocept-derived mesna adduct was clearly visible on Cys69 in both molecules A and B, with a possible additional adduct present on Cys62 of molecules A and B (electron density for an adduct on Cys62 was not quite as strong). For the Trx pH 9.0/7.0 structure, a Tavocept-derived mesna adduct was clearly visible on both Cys69 and Cys62 in both molecules A and B. For the Trx pH 7.0/7.0, a Tavocept-derived mesna adduct was clearly visible on Cys69 in both molecules A and B.

### Structure Solution and Refinement

Data was indexed, integrated, scaled, and merged using the programs HKL2000 or Mosflm. The structure was solved by molecular replacement with PHASER using a monomer from the Protein Data Bank (PDB) entry for human Trx (PDBID 2HXK) as the search model. The solution obtained was consistent with four molecules in the crystal asymmetric unit. The protein model was iteratively refit and refined using MIFit (MIFit Open Source Project, 2010 http://code.google.com/p/mifit) and REFMAC5 (see, Murshudov G N, Vagin A A, Dodson E J. Refinement of macromolecular structures by the maximum-likelihood method. Acta Crystallogr. D. Biol. Crystallogr. 53:240-255 (1997)). Molecules C and D were substantially rebuilt and the Tavocept-derived mesna adduct added after protein rebuilding was complete. The structure solution was supported by contiguous electron density for the entire chain trace of each molecule, landmark side chain density features matching the amino acid sequence including cysteines, absence of phi-psi violations, and final R/R_{free} values in the normal range. The structure showed an unusual conformation and disulfide formation for two of the four protein molecules in the crystal asymmetric unit. For the Trx pH 9.0/8.5 structure, residual density observed near Cys69 of molecule A was modeled as a Tavocept-derived mesna adduct in a dual conformation. For the Trx pH 9.0/7.0 structure, residue density observed near Cys69 of molecules A and B and Cys62 of molecules A and B was modeled as Tavocept-derived mesna adducts (two orientations for Cys69 adduct; single orientation for Cys62 adduct). For the Trx pH 7.0/7.0 structure, residual density observed near Cys69 of molecules A and B was modeled as a Tavocept-mesna adduct. For all three structures, residual density at the N-terminus was modeled as a Thr residual from the TEV recognition site.

C. X-ray Crystallography Results

### Human Trx Contains Covalent Tavocept-Derived Mesna-Adducts and Has a Unique Tetrameric Assembly with a Scrambled Disulfide Bonding Network

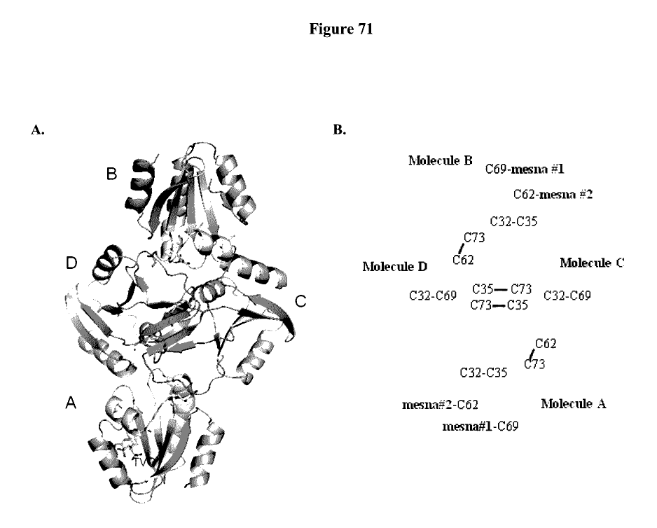

X-ray crystallographic analyses elucidated the three-dimensional structure of human Trx, where adduct formation/crystallization were at the following pH combinations: (i) pH 9.0/8.5; (ii) pH 9.0/7.0; or (iii) pH 7.0/7.0. In all three crystals, Trx adopts a unique tetrameric structure where the disulfide bonding network is scrambled (see, FIG. 71).

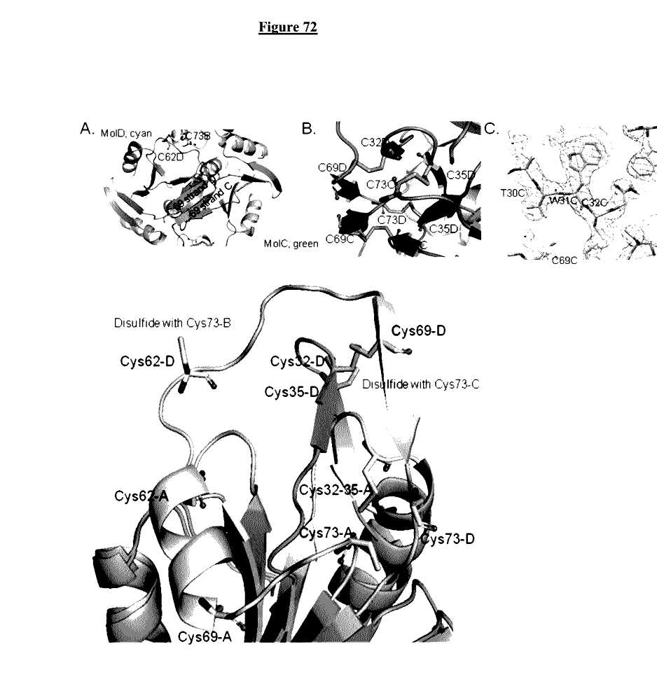

A close-up of the tetramer interface is shown in FIG. 72, Panel A with particular attention to the dimer interface of molecules C and D. The interface between molecules C and D is formed by a 6 stranded β-barrel with three strands coming from each monomer (see, FIG. 72, Panel A). The barrel motif is further stabilized by a series of intramolecular disulfide bonds (i.e., between Cys69 and Cys32 in molecules C and D) and intermolecular disulfides involving Cys35 from molecule C and Cys73 from molecule D, and Cys73 from molecule C and Cys35 from molecule D, resulting in notable disulfide reshuffling. Indeed, all of the cysteine residues in molecules C and D are involved in non-standard (for Trx) disulfide bonds (see, FIG. 71, Panels A and B). The residues that were mutated to increase the p1 and facilitate crystallization under neutral pH and higher pH conditions (i.e., E13K, D16K, E95K and E103K) did not contribute to the observed structural changes.

### Human Trx Contains Covalent Tavocept-Derived Mesna-Adducts on Cys69 and Cys62

The three Trx structures are highly similar (RMSD of 0.533 for Trx pH 9.0/8.5 and Trx 7.0/7.0 and RMSD of 1.08 Å for Trx pH 9.0/7.0 and Trx 7.0/7.0). See, FIG. 72. In the Trx pH 9.0/8.5 structure on molecule B there is density for the sulfoxide near Cys69, but the Tavocept-derived mesna is not clearly connected to the Cys69 in molecule A and, therefore, was not modeled.

The Trx pH 9.0/7.0 structure and the Trx pH 7.0/7.0 structure clearly contain a Tavocept-derived mesna adduct on Cys69 of both molecule A and molecule B. Additionally, The Trx pH 9.0/7.0 structure contains a second Tavocept-derived mesna adduct on Cys62 of molecules A and B. Cys62 is partially buried and appears to be less accessible to modification than Cys69. In this structure, for molecule A only, the sulfonate oxygens of mesna are in potential contact with Ser7 (OG atom). All of these possible interactions may contribute to the binding and/or stabilization of mesna on Trx. The previously reported Mass Spectroscopy results indicated an additional adduct on Cys73 of Trx, but this adduct was not captured in the X-ray crystallographic studies disclosed herein. Cys73 is positioned at the interface of: (i) molecule A/molecule C; (ii) molecule C/molecule D; and (iii) molecule D/molecule B Trx subunits in the tetramer and, therefore, may not be accessible under conditions which favor crystal packing and crystal formation. Indeed in the Trx dimer structure reported by Weichsel and colleagues, Cys73 is also located at the interface between the two Trx molecules and appears to be relatively inaccessible as well in this structure. See, e.g., Weichsel A, Gasdaska J R, Powis G, Montfort W R. Crystal structures of reduced, oxidized, and mutated human thioredoxins: evidence for a regulatory homodimer. Structure 4:735-751 (1996).

### Tavocept Ligand Binding Site

As determined by the electron density maps for the sites of the Tavocept-derived mesna adduct on the Trx structures described herein (i.e., Trx pH 9.0/8.5, Trx pH 9.0/7.0, and Trx 7.0/7.0), molecule A always contains a Tavocept-derived mesna adduct on Cys69. The Trx surface at the site of the Tavocept-derived mesna modification indicates that Cys69 is solvent exposed and the site where the Tavocept-derived mesna adduct binds is large and open allowing the sulfonate moiety of the small mesna adduct to assume at least two distinct conformations.

In addition, as previously discussed, in the pH 9.0/8.5 structure, Phe11 is rotated by 90 degrees to accommodate the mesna modification. Sulfonate oxygens from mesna are in potential hydrogen bond contact with Gln12-ND2 (3.25 Å). Additionally, there is density for the sulfonate near Cys69 on molecule B, but the Tavocept-derived mesna is not clearly connected to the Cys69 and, therefore, was not modeled. The Trx pH 9.0/7.0 structure and the Trx pH 7.0/7.0 structure clearly contain a Tavocept-derived mesna adduct on Cys69 of molecule B. However, Phe11 is not rotated by 90 degrees in these two structures. In the pH 7.0/7.0 structure, in molecule A only, the sulfonate oxygens from mesna may have weak hydrogen bonding interactions with Gln12; this is not observed in the pH 9.0/7.0 structure.

### Conformational Changes in Human Trx Attributed to Covalent Tavocept-Derived Adducts

As noted above (see, FIG. 71), all three Trx structures are highly similar (RMSD of 0.533 for Trx pH 9.0/8.5 and Trx 7.0/7.0 and RMSD of 1.08 Å for Trx pH 9.0/7.0 and Trx 7.0/7.0); therefore, the description of the molecular features herein will focus upon the Trx pH 9.0/7.0 structure, while noting slight variations of the other structures when they arise. The Trx pH 9.0/8.5 structure diffracted to 2.5 Å (C2 space group). The Trx pH 9.0/7.0 structure diffracted to 2.8 Å (C2 space group). The Trx pH 7.0/7.0 structure, diffracted to 1.85 Å resolution (P2₁ space group). All three structures are covalently linked via intermolecular disulfide bonds and exhibit notable conformational changes relative to unmodified Trx (e.g., FIG. 71). Two of the Trx molecules, A and B, in each Trx tetramer are structurally identical to previously reported Trx conformations observed in low pH crystallography experiments. Molecules C and D of each Trx tetramer are in a conformation previously unreported for Trx that is significantly different than molecules A and B (refer to FIG. 71, Panel A). Below is disclosed the main features of Trx molecules A and B, molecules C and D and describe the interfaces between the Trx molecules within the tetramer and ligand binding interactions.

### Description of Molecules A and B in the Tetramer

In all three Trx structures, molecule A contains a Tavocept-derived mesna adduct on Cys69. Phe11 is rotated by 90 degrees to accommodate this modification. In the Trx pH 9.0/8.5 structure on molecule B there is density for the sulfonate near Cys69, but the Tavocept-derived mesna is not clearly connected to the Cys69 and, therefore, was not modeled. The Trx pH 9.0/7.0 structure and the Trx pH 7.0/7.0 structure clearly contain a Tavocept-derived mesna adduct on Cys69 of molecule B. Additionally, The Trx pH 9.0/7.0 structure contains a second Tavocept-derived mesna adduct on Cys62 of molecules A and B. Using the PDB atom nomenclature, hydrogen-bond interactions between the sulfonate oxygens of the Tavocept-derived mesna adduct and NE₂ of Gln12 are possible. As seen in previously reported oxidized structures of human Trx where active site residues Cys32 and Cys35 form a disulfide, the SG atom on Cys32 and backbone N atom on Cys35 of molecule A are involved in hydrogen-bond interactions. There are also possible hydrogen bond interactions between Asp60(OD1) with Trp31(NE1) and Asp26(OD1) with Ser28(OG).

### Interface Between Molecule a and Molecules C and D

Hydrophobic interactions between residues Ala29, Trp31, Val59, Ala66, Val71, and Met74 from molecule A with residues Pro34 and Val65 of molecule C and residues Pro40, Phe41, Met74, Pro75, and Ala92 of molecule D stabilize the interface between molecule A, C, and D. Hydrogen bond interactions are seen between Asp58(OD1) of molecule A and Asn93(ND2) of molecule D, as well as between Thr30(OG1) of molecule A and Ala92(O) and molecule D. Cys73 from molecule A forms an intermolecular disulfide bond with Cys62 of molecule C. In the process, a β-sheet interface between these two molecules is formed by Val71-Met74 (molecule A) and Cys62-Val65 (molecule C). Three additional hydrogen bond interactions involving Ala92(N)-Asp61(O), Gly33(N)-Gln63(OE1) and Met74(N)-Cys62(SG) complete the interface between molecules A and C.

### Conformational Differences in Molecules A and D Versus Molecules C and D

An overlay of molecule A (yellow) and molecule D (cyan) illustrating the conformational change observed is shown in FIG. 72. Specifically, in molecule D the α3 helix containing Cys62 and Cys69 unwinds to form an extended loop strand motif. Cys62 from molecule D forms an intermolecular disulfide with Cys73 from molecule B and an intramolecular hydrogen bond with Cys32 in molecule D. The active site loop containing Cys32 and Cys35 unwinds to form a two stranded β-turn with Cys35 of molecule D making an intramolecular disulfide with Cys73.

### Description of Molecules C and D in the Tetramer

As mentioned previously, molecule C and D in the Trx tetramer are structurally distinct in comparison to previously reported Trx-1 (PDB ID: IERU) with notable changes in the α2 and α3 helices (see, FIG. 71, Panel A). In molecule C, residues Ala29, Thr30, and Trp31, which are normally part of a β-turn connecting β2 and α2 in Trx-1 (IERU), become a β-strand. Residues Lys36, Met37 and Ile38, which are normally part of α2, become a β-strand. These newly formed β-strands interact via hydrogen-bond interactions between Thr30 and Met37, forming an anti-parallel β-sheet. A type II′ β-turn formed by Cys32, Gly33, Pro34, and Cys35 residues facilitate formation of this β-sheet. The methylene side chain groups of Lys36 interact in a hydrophobic fashion with the side chains of Trp31 and Ile38. A similar β-sheet, involving the same residues, also occurs in Molecule D. The newly formed β-strand (residues Lys36-Ile38) from molecules C and D interact to form an anti-parallel β-sheet with a tight interface between the two molecules. The side chain of Ile38 from molecules C and D orient towards each other and provide hydrophobic contacts to the interface.

In both molecule C and D, residues Gln63-Glu68, which are normally part of α3, become two β-strands (residues Cys62-Asp64 and Glu68-Lys72) connected by a loop. The first β-strand (residues Cys62-Asp64) from molecule C interacts with molecule A (β-strand formed by Val71-Met74). Similarly in molecule D, the first β-strand (residues Cys62-Asp64) interacts with molecule B (residues Val71-Met74). Residues Glu68-Lys72, from molecule C and molecule D, interact with each other forming an anti-parallel β-sheet completing the tight interface between molecules C and D. Hydrophilic residues from molecules C and D (e.g., Glu68, Glu70, Lys72) orient towards the solvent accessible surface whereas, as would be expected the hydrophobic residues orient towards the interior and provide hydrophobic contacts and stability to the interface. Hydrogen bond interactions are completed by Cys35(N)-Cys73(SG), Ser67(SG)-Met74(SD), Cys73(N)-Ser67(O), Cys73(SG)-Cys35(N), Met74(SD)-Ser67(OG), and Ser67(O)-Cys73(N).

V. Summary of Tavocept-Related Structure/Function Data with Thioredoxin
Native PAGE Detects Tavocept-derived Mesna Adducts on Trx.
IEF PAGE Detects Tavocept-derived Mesna Adducts on Trx.
Tavocept-derived mesna adducts form on Cys62/Cys69 and Cys73 on human Trx and were identified using Trypsin digests and Mass Spectroscopy.
Enzyme activity assays indicate that Tavocept and Tavocept-derived mesna-disulfide heteroconjugates act as alternative substrate-inhibitors of Trx in the classical Trx/thioredoxin reductase coupled activity assay.
Enzyme activity assays indicate that modification of human Trx 1 by Tavocept-derived mesna moieties impairs catalytic activity of Trx in the classical Trx/thioredoxin reductase coupled activity assay.
X-ray crystallographic studies (Zenobia) have unequivocally identified Tavocept-derived mesna mixed disulfides on human Trx 1 at Cys62 and Cys69 in a unique tetrameric structure.

(iii) Glutathione and Glutaredoxin System

Glutathione (GSH) is the predominant nonprotein thiol in cells where it plays essential roles as an enzyme substrate and a protecting agent against xenobiotic compounds and oxidants. See, e.g., Dickinson, D. A., Forman, H. J. Cellular glutathione and thiol metabolism. Biochem. Pharmacol. 64:1019-1026 (2002). Glutathione, maintained in the reduced state by glutathione reductase, is able to transfer its reducing equivalents to several enzymes, such as glutathione peroxidases (GPx), glutathione transferases (GSTs), and glutaredoxins. The latter, similar to thioredoxin, can interact with ribonucleotide reductase and with several other proteins involved in cellular signaling and transcription control, such as NF-κB, PTP-1B, PKA, PKC, Akt, and ASK1. See, e.g., Lu, J., Chew, E. H., Holmgren, A. Targeting thioredoxin reducatse is a basis for cancer therapy. Proc. Natl. Acad. Sci. USA 104:12288-12293 (2007). Mammalian cells contain a cytosolic (Grx1) and a mitochondrial (Grx2) glutaredoxin. Mitochondria contain a second glutaredoxin (Grx5), which is homologous to yeast Grx5 in bearing a single cysteine residue at its active site.

The formation of mixed disulfides between protein cysteine residues and glutathione constitutes a protective mechanism for thiols, which prevents their further oxidation in addition to possible roles in cell signaling. Mixed disulfides are derived from the reaction of sulfenic acids in proteins and glutathione rather than from direct interaction of glutathione disulfide and protein thiols. Glutaredoxins play a critical role in the reversible formation of protein mixed disulfides, as they are able to catalyze both the reduction and the formation of mixed disulfides from protein thiols and reduced glutathione. Hence, they may act as sensors of the glutathione redox state.

Several systems are sensitive to glutathionylation, including mitochondrial complex I, which, in this way, increases the production of the superoxide anion. Johansson, et al., found that mitochondrial glutaredoxin is reduced also by thioredoxin reductase, demonstrating that glutathione and thioredoxin pathways are linked. See, Johansson, C, Lillig, C. H., et al. Human mitochondrial glutaredoxin reduces S-glutathionylated proteins with high affinity accepting electrons from either glutathione or thioredoxin reductase. J. Biol. Chem. 279: 7537-7543 (2004).

Glutaredoxin (Grx) has been demonstrated to be over-expressed in cancer cells (see, e.g., Nakamura, H., Bal, J., et al. Expression of thioredoxin and glutaredoxin, redox-regulating proteins, in pancreatic cancer. Cancer Detect. Prevent. 24:53-60 (2000)) and protects from apoptosis (see, e.g., Daily, D., Vlamis, A., et al. Glutaredoxin protects cerebellar granule neurons from dopamine-induced apoptosis by dual activation of the ras-phosphoinositide 3-kinase and jun n-terminal kinase pathways. J. Biol. Chem. 276:21618-21626 (2001)), while silencing the expression of Grx-2 by RNAi sensitize cells to apoptosis-inducing agents (see, e.g., Lillig, H., Lonn, M. E., et al. Short interfering RNA-mediated silencing of glutaredoxin 2 increases the sensitivity of HeLa cells toward doxorubicin and phenylarsine oxide. Proc. Natl. Acad. Sci. USA 101:13227-13232 (2004)). Thioredoxin, glutaredoxin, and Ref-1 favor the DNA binding of several transcription factors by maintaining crucial cysteines in a reduced state. See, e.g., Morel, Y., Barkoui, R. Repression of gene expression by oxidative stress. Biochem. J. 342:481-496 (1999). Although thioredoxin and glutathione systems are apparently similar in their cellular functions as they maintain a reduced environment by using the same source of reducing equivalents (NADPH), a major difference is represented by the cell concentrations of glutathione that are far larger than that of thioredoxin. Nevertheless, the two systems operate independently, fulfilling different roles within the cell. See, e.g., Trotter, E. W., Grant, C. M. Non-reciprocal regulation of the redox state of the glutathione-glutaredoxin and thioredoxin systems. EMBO Rep. 4:184-188 (2004). The presence in the cell of different proteins exhibiting the thioredoxin fold underlines their specific, multiple signaling role. See, e.g., Patwari, P., Lee, R. T. Thioredoxins, mitochondria, and hypertension. Am. J. Pathol. 170:805-808 (2007).

### Glutathione

Glutathione (GSH), a tripeptide (γ-glutamyl-cysteinyl-glycine) serves a highly important role in both intracellular and extracellular redox balance. It is the main derivative of cysteine, and the most abundant intracellular non-protein thiol, with an intracellular concentration approximately 10-times higher than other intracellular thiols. Within the intracellular environment, glutathione (GSH) is maintained in the reduced form by the action of glutathione reductase and NADPH. Under conditions of oxidative stress, however, the concentration of GSH becomes markedly depleted. Glutathione functions in many diverse roles including, but not limited to, regulating antioxidant defenses, detoxification of drugs and xenobiotics, and in the redox regulation of signal transduction. As an antioxidant, glutathione may serve to scavenge intracellular free radicals directly, or act as a co-factor for various other protection enzymes. In addition, glutathione may also have roles in the regulation of immune response, control of cellular proliferation, and prostaglandin metabolism. Glutathione is also particularly relevant to oncology treatment because of its recognized roles in tumor-mediated drug resistance to chemotherapeutic agents and ionizing radiation. Glutathione is able to conjugate electrophilic drugs such as alkylating agents and cisplatin under the action of glutathione S-transferases. Recently, GSH has also been linked to the efflux of other classes of agents such as anthracyclines via the action of the multidrug resistance-associated protein (MRP). In addition to drug detoxification, GSH enhances cell survival by functioning in antioxidant pathways that reduce reactive oxygen species, and maintain cellular thiols (also known as non-protein sulfhydryls (NPSH)) in their reduced states. See, e.g., Kigawa J, et al. Gamma-glutamyl cysteine synthetase up-regulates glutathione and multidrug resistance-associated protein in patients with chemoresistant epithelial ovarian cancer. Clin. Cancer Res. 4:1737-1741 (1998).

Cysteine, another important NPSH, as well as glutathione are also able to prevent DNA damage by radicals produced by ionizing radiation or chemical agents. Cysteine concentrations are typically much lower than GSH when cells are grown in tissue culture, and the role of cysteine as an in vivo cytoprotector is less well-characterized. However, on a molar basis cysteine has been found to exhibit greater protective activity on DNA from the side-effect(s) of radiation or chemical agents. Furthermore, there is evidence that cysteine concentrations in tumor tissues can be significantly greater than those typically found in tissue culture.

A number of studies have examined GSH levels in a variety of solid human tumors, often linking these to clinical outcome See, e.g., Hochwald, S. N., et al. Elevation of glutathione and related enzyme activities in high-grade and metastatic extremity soft tissue sarcoma. American Surg. Oncol. 4:303-309 (1997); Ghazal-Aswad, S., et al. The relationship between tumour glutathione concentration, glutathione S-transferase isoenzyme expression and response to single agent carboplatin in epithelial ovarian cancer patients. Br. J. Cancer 74:468-473 (1996); Berger, S. J., et al. Sensitive enzymatic cycling assay for glutathione: Measurement of glutathione content and its modulation by buthionine sulfoximine in vivo and in vitro human colon cancer. Cancer Res. 54:4077-4083 (1994). Wide ranges of tumor GSH concentrations have been reported, and in general these have been greater (i.e., up to 10-fold) in tumors compared to adjacent normal tissues. Most researchers have assessed the GSH content of bulk tumor tissue using enzymatic assays, or GSH plus cysteine using HPLC.

In addition, cellular thiols/non-protein sulfhydryls (NPSH), e.g., glutathione, have also been associated with increased tumor resistance to therapy by mechanisms that include, but are not limited to: (i) conjugation and excretion of cancer treating agents; (ii) direct and indirect scavenging of reactive oxygen species (ROS) and reactive nitrogen species (RNS); and (iii) maintenance of the “normal” intracellular redox state. Low levels of intracellular oxygen within tumor cells (i.e., tumor hypoxia) caused by aberrant structure and function of the associated tumor vasculature, has also been shown to be associated with chemotherapy therapy-resistance and biologically-aggressive malignant disease. Oxidative stress, commonly found in regions of intermittent hypoxia, has been implicated in regulation of glutathione metabolism, thus linking increased NPSH levels to tumor hypoxia. Therefore, it is also important to characterize both NPSH expression and its relationship to tumor hypoxia in tumors and other neoplastic tissues.

The heterogeneity of NPSH levels was examined in multiple biopsies obtained from patients with cervical carcinomas who were entered into a study investigating the activity of cellular oxidation and reduction levels (specifically, hypoxia) on the response to radical radiotherapy. See, e.g., Fyles, A., et al. (Oxygenation predicts radiation response and survival in patients with cervix cancer. Radiother. Oncol. 48:149-156 (1998). The major findings from this study were that the intertumoral heterogeneity of the concentrations of GSH and cysteine exceeds the intratumoral heterogeneity, and that cysteine concentrations of approximately 21 mM were found in some samples, confirming an earlier report by Guichard, et al. (Glutathione and cysteine levels in human tumour biopsies. Br. J. Radiol. 134:63557-635561 (1990)). These levels of cysteine are much greater than those typically seen in tissue culture, suggesting that cysteine might exert a significant radioprotective activity in cervical carcinomas and possibly other types of cancer.

There is also extensive literature showing that elevated cellular glutathione levels can produce drug resistance in experimental models, due to drug detoxification or to the antioxidant activity of GSH. In addition, radiation-induced DNA radicals can be repaired non-enzymatically by GSH and cysteine, indicating a potential role for NPSH in radiation resistance. While cysteine is the more effective radioprotective agent, it is usually present in lower concentrations than GSH. Interestingly, under fully aerobic conditions, this radioprotective activity appears to be relatively minor, and NPSH compete more effectively with oxygen for DNA radicals under the hypoxic conditions that exist in some solid tumors, which might play a significant role in radiation resistance.

Radiotherapy has traditionally been a major treatment modality for cervical carcinomas. Randomized clinical trials (Rose, D., et al. Concurrent cisplatin-based radiotherapy and chemotherapy for locally advanced cervical carcinoma. New Engl. J. Med. 340:1144-1153 (1999)) show that patient outcome is significantly improved when radiation therapy is combined with cisplatin-based chemotherapy, and combined modality therapy is now widely being utilized in treatment regimens. It is important to establish the clinical relevance of GSH and cysteine levels to drug and radiation resistance because of the potential to modulate these levels using agents such as buthionine sulfoximine; an irreversible inhibitor of γ-glutanylcysteine synthetase that can produce profound depletion of GSH in both tumor and normal tissues. See, e.g., Bailey, T., et al. Phase I clinical trial of intravenous buthionine sulfoximine and melphalan: An attempt at modulation of glutathione. J. Clin. Oncol. 12:194-205 (1994). Evaluation of GSH concentrations have reported elevated tumor GSH relative to adjacent normal tissue, and intertumoral heterogeneity in GSH content. These findings are consistent with the idea that GSH could play a clinically significant role in drug resistance. although it should be noted that relatively few studies have the sample size and follow up duration necessary to detect a significant relation between tumor GSH content and response to chemotherapy, hence there are no consistent clinical data to support this idea.

Koch and Evans (Cysteine concentrations in rodent tumors: unexpectedly high values may cause therapy resistance. Int. J. Cancer 67:661-667 (1996)) have shown that cysteine concentrations in established tumor cell lines can be much greater when these are grown as in vivo tumors, as compared to the in vitro values, suggesting that cysteine might play a more significant role in therapy resistance than previously considered. Although relatively few studies have reported on cysteine levels in human cancers, an earlier HPLC-based study of cervical carcinomas by Guichard, D. G., et al. (Glutathione and cysteine levels in human tumour biopsies. Br. J. Radiol. 134:63557-635561 (1990)) reported cysteine concentrations greater than 1 mM in a significant number of cases. Thus, the fact that the variability in cysteine levels is greater than that for GSH suggests that these two thiols are regulated differently in tumors. By way of non-limiting example, the inhibition of γ-glutamylcysteine synthetase with the intravenous administration of buthionine sulfoximine (BSO) could result in elevated cellular levels of cysteine, due to the fact that the γ-glutamylcysteine synthetase is not being utilized for GSH de novo synthesis. Similar to GSH, cysteine possesses the ability to repair radiation-induced DNA radicals and cysteine also has the potential to detoxify cisplatin; a cytotoxic agent now routinely combined with radiotherapy to treat locally-advanced cervical carcinomas.

Glutaredoxin

Glutaredoxin (Grx), like thioredoxin (Trx), are members of the thioredoxin superfamily that mediate disulfide exchange via their Cys-containing catalytic sites. While glutaredoxins mostly reduce mixed disulfides containing glutathione, thioredoxins are involved in the maintenance of protein sulfhydryls in their reduced state via disulfide bond reduction. See, e.g., Print, W. A., et al. The role of the thioredoxin and glutaredoxin pathways in reducing protein disulfide bonds in the Escherichia coli cytoplasm. J. Biol. Chem. 272:15661-15667 (1996). The reduced form of thioredoxin is generated by the action of thioredoxin reductase; whereas glutathione provides directly the reducing potential for regeneration of the reduced form of glutaredoxin.

Glutaredoxins are small redox enzymes of approximately 100 amino acid residues, which use glutathione as a cofactor. Glutaredoxins are oxidized by substrates, and reduced non-enzymatically by glutathione. In contrast to thioredoxins, which are reduced by thioredoxin reductase, no oxidoreductase exists that specifically reduces glutaredoxins. Instead, oxidized glutathione is regenerated by glutathione reductase. Together these components comprise the glutathione system. See, e.g., Holmgren, A. and Fernandes, A. P., Glutaredoxins: glutathione-dependent redox enzymes with functions far beyond a simple thioredoxin backup system. Antioxid. Redox. Signal. 6:63-74 (2004); Holmgren, A., Thioredoxin and glutaredoxin systems. J. Biol. Chem. 264:13963-13966 (1989).

Glutaredoxins basically function as electron carriers in the glutathione-dependent synthesis of deoxyribonucleotides by the enzyme ribonucleotide reductase. Like thioredoxin, which functions in a similar way, glutaredoxin possesses an active catalytic site disulfide bond. It exists in either a reduced or an oxidized form where the two cysteine residues are linked in an intramolecular disulfide bond. Human proteins containing this domain include: glutaredoxin thioltransferase (GLRX); glutaredoxin 2 (GLRX2); thioredoxin-like 2 (GLRX3); GLRX5; PTGES2; and TXNL3. See, e.g., Nilsson, L. and Foloppe, N., The glutaredoxin -C-P-Y-C-motif: influence of peripheral residues. Structure 12:289-300 (2004).

At least two glutaredoxin proteins exist in mammalian cells (12 or 16 kDa), and glutaredoxin, like thioredoxin, cycles between disulfide and dithiol forms. The conversion of glutaredoxin from the disulfide form (oxidized) to the dithiol (reduced) form is catalyzed non-enzymatically by glutathione and is illustrated, below. In turn, glutathione cycles between a thiol form (glutathione) that can reduce glutaredoxin and a disulfide form (glutathione disulfide); glutathione reductase enzymatically reduces glutathione disulfide to glutathione. This reaction is illustrated below:

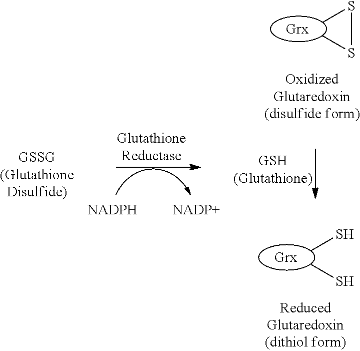

While the -CysXaaXaaCys-intramolecular disulfide bond is an essential part of the catalytic cycle for thioredoxin and protein disulfide isomerase, the most important oxidized species for glutaredoxins is a glutathionylated form.

### Control of Cell Thiol Redox State by Thioredoxin and Glutathione Systems

The thiol redox control concept was introduced to indicate the signaling action of the thioredoxin system on the thiol enzyme activity. See, e.g., Holmgren, A., Johansson, C., et al. Thiol redox control via thioredoxin and glutaredoxin systems. Biochem. Soc. Trans. 33:1375-1377 (2005). The cellular thiol redox state is controlled by two major systems, the thioredoxin and glutathione systems, which are in a close redox communication with hydrogen peroxide through peroxiredoxins and glutathione peroxidases, respectively. They are present both in the cytosol and mitochondria and, in either system, the reducing equivalents are fed by NADPH. Different pathways of NADP⁺ reduction are operative in the cytosol versus mitochondria. Whereas cytosolic NADP⁺ is reduced in the pentose phosphate pathway, in mitochondria, electrons are delivered through the various dehydrogenases coupled to the energy-linked transhydrogenase that catalyzes the transfer of reducing equivalents from NADH to NADP⁺. Furthermore, the mitochondrial glutamate and isocitrate dehydrogenases, in addition to NAD⁺, use NADP⁺ for the oxidation of their respective substrates, providing a further source of NADPH.

By way of non-limiting example, the reduction of hydrogen peroxide (H₂O₂) as mediated by thioredoxin (A) and glutathione (B) pathways is illustrated below in Table 23. Electrons are delivered by NADPH maintained reduced by the pentose phosphate pathway in the cytosol, and by the respiratory substrates in mitochondria. The proton-translocating trans-hydrogenase transfers electrons from NADH to NADP⁺ to form NADPH. It should be noted that both sulfenic and selenenic acid residues appear as key intermediates in the thioredoxin and glutathione pathways, respectively.

TABLE 23
The Reduction of Hydrogen Peroxide (H₂O₂) by Thioredoxin (A) and Glutathione (B) Pathways

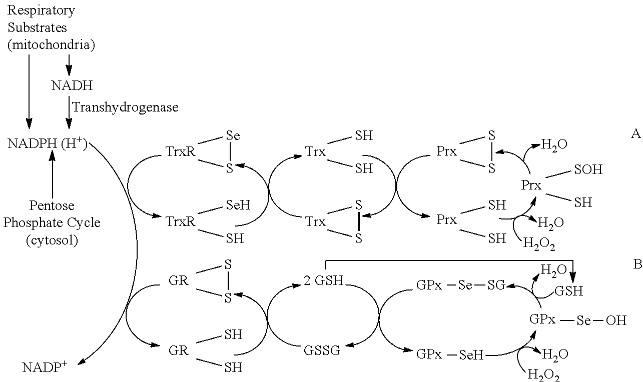

### Specific Examples and Experimental Results of Tavocept-Related Studies on Glutaredoxin (Grx)

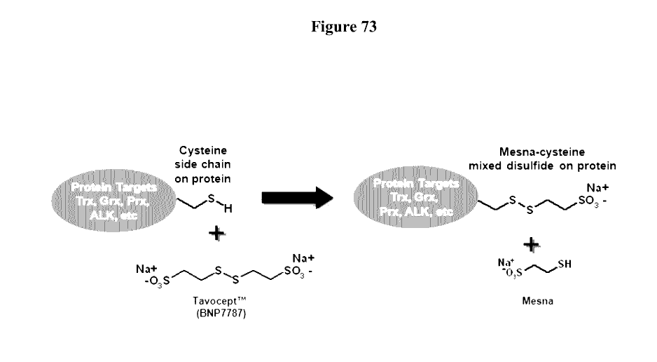

The following studies were designed to determine if Tavocept forms a detectable, covalent modification(s) on Glutaredoxin (Grx). Specifically, these studies address whether Tavocept can undergo thiol-disulfide exchange with selected cysteine residues on Grx resulting in formation of a Tavocept-derived mesna-cysteine mixed disulfide. See, FIG. 73. In addition, experiments described in the following sections unequivocally confirm that Tavocept forms mixed-disulfides with cysteine (Cys) residues of human Grx, specifically a Tavocept-derived mesna adduct is formed with Cys7 and Cys82.

In brief, wild-Type human glutaredoxin (Grx1) was cloned into a proprietary vector containing an N-terminal 6×his tag cleavable by TEV protease using the following primers: 5′-TATATA GGT ACC GCT CAA GAG TTT GTG AAC-3′ and 5′-TATATA GGA TCC TCA CTG CAG AGC TCC AA-3′. Final product was verified by DNA sequencing.

The final product was expressed in BL21 (RIPL) cells. Cells containing the human GRX1 construct were grown at 37° C. to OD₆₀₀˜0.6. The cells were induced with 0.5 mM IPTG at 18° C. overnight. The cell biomass was harvested and stored at −80° C. until ready to use. Purification of target protein was performed in a three column system, as follows. The cell biomass was initially lysed by sonication in 50 mM Tris-HCl pH 7.8, 500 mM NaCl, 10% Glycerol, 20 mM Imidazole, 5 mM BME (Buffer A) plus 1 Roche Complete Protease Inhibitor Tablet, and 20,000 units Benzonase. Target protein was extracted by binding to Ni2+ charged IMAC resin and eluted with 250 mM Imidazole. Peak fractions were cleaved with 3 mg TEV overnight in Buffer A. Cleaved protein was then run over Ni2+ charged IMAC resin and the flow-through was collected. Aggregated protein was separated from monomeric protein via Size Exclusion (S-75) in 50 mM Tris-HCl pH7.5, 250 mM NaCl, and 5 mM DTT. The monomeric protein was concentrated to ˜39 mg/mL.

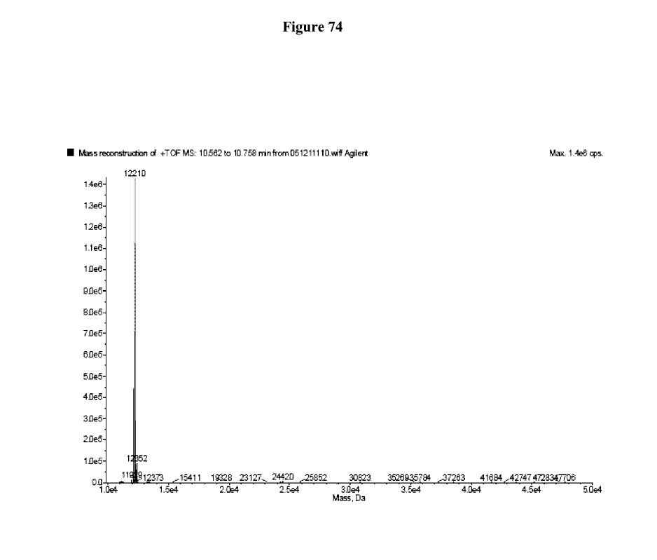

Tavocept-derived mesna adduct on Grx was prepared by a protocol developed by the Applicant at BioNumerik Pharmaceuticals, Inc. Grx was reduced with DTT for 1 hour at 30° C. and then further overnight at 4° C. Excess DTT was removed by exchanging 5-times in 50 mM Tris pH 7.5, 250 mM NaCl using ultracentrifugation. Next Grx was supplemented with 1 mM DTPA, 1 mM Neocuprione, and 40 mM Tavocept and incubated at 4° C. overnight. The Grx-Tavocept reaction was then characterized by Mass Spectroscopy (MS) for the presence of a Tavocept-derived mesna adduct. MS analysis suggested that protein going into crystallization had one to two Tavocept-derived mesna adducts per molecule. See, FIG. 74. Fine screening was done with various crystallization conditions at different protein concentration and different protein and reservoir ratios to obtain diffraction quality crystals. Before data collection, the crystals were transferred into a cryoprotectant solution made up of 25% ethylene glycol (v/v) in crystallization buffer, after which they were flash-frozen in liquid nitrogen for data collection. The crystals grown under conditions of 20% PEG 8K, 0.1 M Phosphate citrate pH 4.2, 0.2 M NaCl) diffracted to better than 1.1 Å.

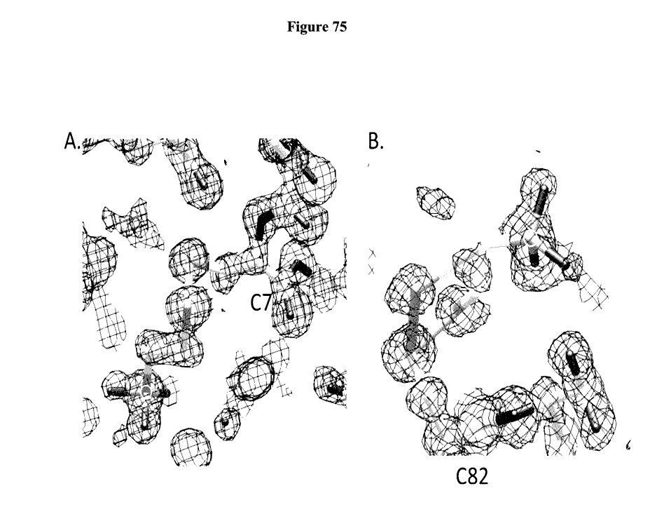

Diffraction data were collected at the Advanced Light Source (ALS) (Berkeley, Calif.). Tavocept-derived mesna adducts were observed on Cys7 and Cys82 and clearly defined in the atomic resolution map. Data was processed using the program package MosFlm as part of the ccp4 program package. Table 24 below summarizes the image processing statistics. The final electron density maps for the Tavocept-derived mesna adducts are shown in FIG. 75.

TABLE 24
Crystal Characteristics and Data Collection Statistics
(outer shell statistics in parenthesis)

| Parameter | Value |
|---|---|
| Unit cell (Å, °) | 27.847 55.414 130.357 90.000 90.000 90.000 |
| Space group | C2221 |
| Resolution range (Å) | 27.71-1.08 (1.13-1.08) |
| No. of observations | 165287 |
| No. of unique reflections | 31162 |
| Redundancy | 5.3 (2.5) |
| Completeness (%) | 70.4 (13.2) |
| Mean I/sigma(I) | 19.5 (2.3) |
| Rmerge | 0.051 (0.450) |

**Note:
The low completeness in the outer shell was due to limits in detector geometry and not limits in the diffraction. All reflections were included in the refinement to provide the highest quality map.

Data was indexed, integrated, scaled and merged using the program Mosflm. The structure was solved by molecular replacement with Phaser using a monomer from the Protein Data Bank entry for human Grx (PDBID 1KTE). The solution was consistent with one molecule in the crystal asymmetric unit. The protein model was iteratively refit and refined using MIFit (MIFit Open Source Project, 2010) and REFMAC5 (Murshudov, et al., 1997). The structure solution is supported by contiguous electron density for the entire chain trace of each molecule, landmark side chain density features matching the amino acid sequence including cysteines, absence of phi-psi violations and final R/R_{free} values in the normal range. Residual density observed near Cys7 and Cys82 was modeled as Tavocept-derived mesna adducts. Residues Gln39 and Glu55 have missing side-chain atoms in the final structures (side chain atoms CD, CG, OE1, NE2 and CD, OE1, OE2, respectively). Table 25 summarizes the final refinement statistics.

TABLE 25
Crystallographic Data and Refinement Statistics

| Parameter | Value |
|---|---|
| Resolution range (Å) | 27.707-1.076 |
| No. of reflections | 31116 (29546 working set, 1570 test set) |
| No. of protein chains | 1 (A) |
| Ligand id codes | UNK |
| No. of protein residues | 107 |
| No. of ligands | 2 |
| No. of waters | 202 |
| No. of atoms | 1077 |
| Mean B-factor | 12.733 |
| Rwork | 0.1722 |
| Rfree | 0.1952 |
| Rmsd bond lengths (Å) | 0.008 |
| Rmsd bond angles (°) | 1.156 |
| Number of disallowed φψ | 0 |
| angles | |

### Structure of Human Grx Structure Modified by Tavocept

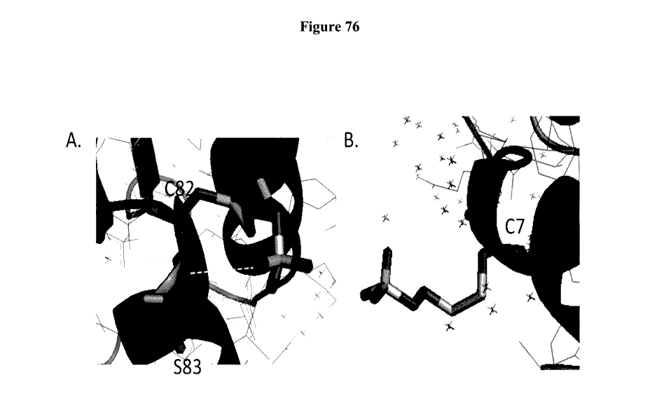

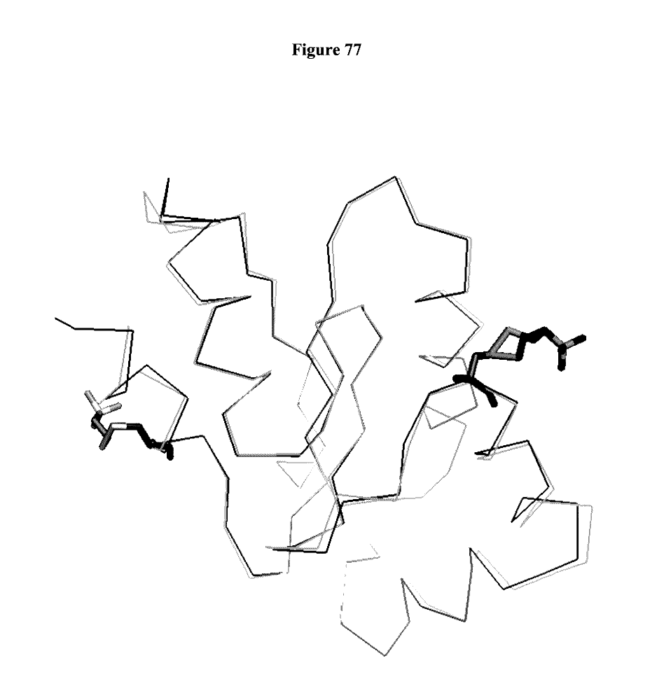

The crystal structure of Grx1 in complex with a Tavocept-derived mesna moiety has been completed at atomic resolution. The protein crystallizes with a monomer in the asymmetric unit. Tavocept-derived mesna moieties were observed at Cys7 and Cys82. See, FIG. 76. Both ligand binding sites are solvent accessible. The Tavocept-derived mesna adducts are located at a crystal contact and within close proximity of each other. See, FIG. 77.

### Summary of Human Grx with Tavocept-Derived Mesna Adducts

Mass spectroscopy and x-crystal structure analysis of human Grx1 has been completed in complex with a Tavocept-derived mesna moiety at atomic resolution.
Mass spectrometry data suggested that the protein after reaction with Tavocept contained up to two Tavocept-derived mesna adducts.
When crystals with adducts were dissolved and analyzed by mass spectrometry, the monomer appeared to contain up to two adducts.
Tavocept-derived mesna adduct was found at Cys7 and Cys82 on Human Grx.
Both Tavocept-derived mesna adducts on Human Grx are solvent exposed, but clearly defined in the electron density map.

D. Prenyltransferases

Human protein prenyltransferases include the proteins farnesyltransferase (FTase), geranylgeranyltransferase I (GGTase I), and geranylgeranyltransferase II (GGTase II). These prenyltransferases transfer lipophilic isoprene groups that enable the prenylated substrates to more avidly associate with cellular membranes. The proteins that are prenylated by the human protein prenyltransferases are involved in a range of intracellular pathways and processes important for cell growth and proliferation. See, e.g., Holstein and Hohl, Is there a future for prenyltransferases inhibitors in cancer therapy? Curr. Opin. Pharmacol. 12:704-709 (2012); Maurer-Stroh, et al., Protein prenyltransferases. Genome Biol. 4:212-221 (2003). Although cancer treating agents that specifically target prenyltransferases have not yet received FDA approval in the United States, prenyltransferases represent attractive targets for drug discovery especially within the area of oncology. See, e.g., Holstein and Hohl, Is there a future for prenyltransferases inhibitors in cancer therapy? Curr. Opin. Pharmacol. 12:704-709 (2012). Targeting prenyltransferases requires a global cellular perspective. For example, inhibition of the prenyltransferases, FTase and GGTase I alone, might not be an effective anti-cancer approach were it not for the fact that the substrates that are post-translationally modified by these prenyltransferases are essential in regulating many different cell growth and cell survival signaling pathways. A specific example of FTase- and GGTase-mediated prenylation that is important and required for the regulation of cell proliferation and cell survival involves the RAS protein family.

By way of non-limiting example, RAS proteins include: KRAS, HRAS, and NRAS. RAS proteins have high sequence similarity/identity and regulate proteins that have important roles in cell proliferation-related pathways, including but not limited to, MAPK, STAT, Raf, MEK, and ERK; as well as proteins that are key in anti-apoptotic pathways, including but not limited to, PI3K and Akt. See, e.g., Vadakara and Borghael, Personalized medicine and treatment approaches in non-small-cell lung carcinoma. Pharmacogenomics Personalized Med. 5:113-123 (2012); Riely, et al., KRAS mutations in non-small cell lung cancer. Proc. Am. Thorac. Soc. 6:201-205 (2009). RAS protein mutations and/or functional dysregulation has been implicated in up to one-third of all human cancers. See, e.g., Baines, et al., Inhibition of Ras for cancer treatment: the search continues. Future Med. Chem. 3:(14) 1787-1808 (2011); Santarpia, et al., Targeting the mitogen-activated protein kinase RAS-RAF signaling pathway in cancer therapy. Expert Opin. Ther. Targets 16(1):113-119 (2012). For example, KRAS is an important oncology target that is commonly mutated in 80% of pancreatic cancer patients, 20% of all non-small cell lung cancer (NSCLC) patients, and is also often mutated in colorectal cancer patients as well. See, e.g., Adjei, Blocking onocogenic Ras signaling for cancer therapy. J. Natl. Cancer Inst. 93:(14) 1062-1074 (2001); Johnson and Heymach, Farnesyl transferase inhibitors for patients with lung cancer. Clin. Cancer. Res. 10:4254s-4257s (2004); Baines, et al., Inhibition of Ras for cancer treatment: the search continues. Future Med. Chem. 3(14):1787-1808 (2011). RAS proteins are substrates for prenyltransferases and, regardless of their mutational state, must be prenylated to be able to translocate to the cell membrane and transduce signals that regulate cell proliferation and apoptosis. See, e.g., Sebti, Blocked pathways: FTIs shut down oncogene signals. The Oncologist 8(Suppl 3):30-38 (2003). As a consequence of these important activities, proteins that prenylate RAS, such as farnesyltransferase (FTase) and geranylgeranyltransferase (GGTase), are attractive targets for anti-cancer drug development efforts.

Members of the RAS protein family are substrates for both FTase and GGTase I and effective inhibitors of RAS, which work by inhibiting prenylation and, therefore, localization to the membrane, must inhibit both FTase and GGTase I. Given the fact that RAS proteins are important in NSCLC (see, e.g., Vadakara and Borghael, Personalized medicine and treatment approaches in non-small-cell lung carcinoma. Pharamcogen. Personalized Med. 5:113-123 (2012); Riely, et al., KRAS mutations in non-small cell lung cancer. Proc. Am. Thorac. Soc. 6:201-205 (2009); Johnson and Heymach, Farnesyl transferase inhibitors for patients with lung cancer. Clin. Cancer Res. 10:4254s-4257s (2004)) as well as in pancreatic, colorectal, and other cancers (see, e.g., Baines, et al., Inhibition of Ras for cancer treatment: the search continues. Future Med. Chem. 3(14):1787-1808 (2011)), the development of compounds that modulate the function of prenyltransferases like FTase, which in turn modulate the function of both wild type and mutated RAS proteins, are clearly important.

(i) Farnesyltransferase

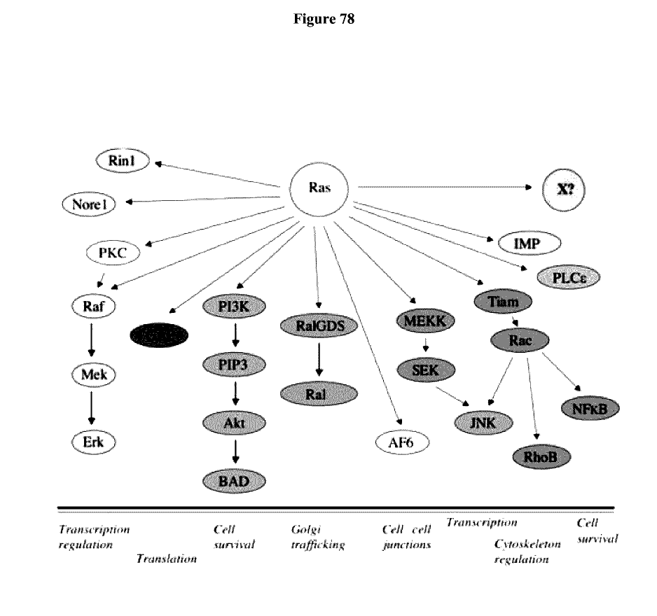

Farnesyltransferase (FTase) catalyzes the addition of a 15 carbon moiety onto key proteins, including, but not limited to: (i) the RAS family of proteins; (ii) kinetochore proteins; (iii) cGMP phosphodiesterase; (iv) peroxisomal proteins; (v) nuclear lamina proteins; (vi) heat shock homologs; (vii) rhodopsin kinase; and similar proteins. See, e.g., Maurer-Stroh, et al., Protein prenyltransferases. Genome Biol. 4:212-221 (2003). A key target of FTase is the RAS protein family (e.g., HRAS, KRAS and NRAS). RAS modulates a wide range of intracellular signaling pathways the regulate cell growth, cell proliferation, and apoptosis. See, FIG. 78; Appels, et al., Development of Farnesyl Transferase Inhibitors: A Review. 10:565-578 (2005).

### Specific Examples and Summary of Experimental Results of Tavocept-Related Studies on Human Farnesyltransferase

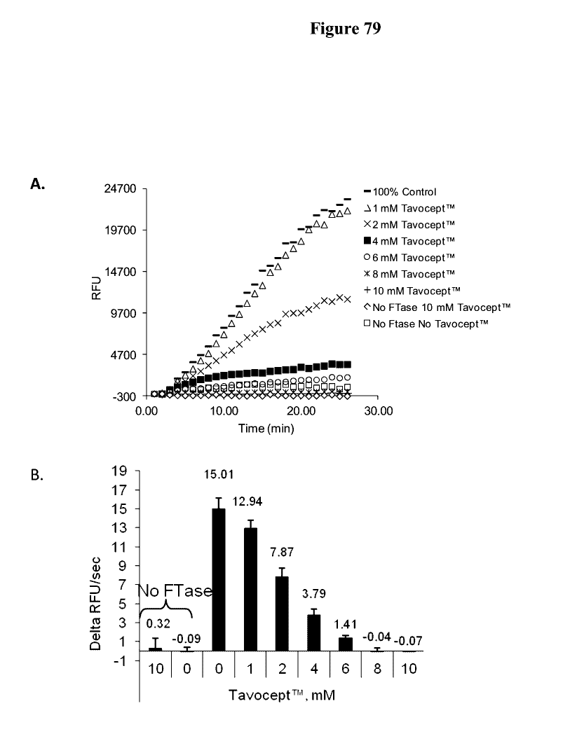

Tavocept-inhibited human farnesyltransferase-mediated transfer of farnesylpyrophosphate to the cysteine residue in a danyslated peptide substrate of sequence glycine-cysteine-valine-leucine-serine (designated—Dansyl-GCVLS) in vitro. See, FIG. 79. The assay reactions contained 25 nM recombinant human farnesyltransferase (Abcam), 10 μM farnesylpyrophosphate (SigmaAldrich), 3 μM Dansyl-GCVLS (NeoBioScience) in Tris buffer (50 mM, pH 7.5) containing 5 mM MgCl₂, 10 μM ZnCl₂, and 0.2% octyl-β-D-glucopyranoside. Total assay volumes were 50 μL and the reactions were initiated by the addition of FTase (5 μL of 250 nM stock was added to 45 μL of assay components listed above). The excitation wavelength was 340 nm (a 340 nm filter with 25 nm bandpass) and the emission wavelength was 505 nM (505 nm filter with 20 nm bandpass) and the assay temperature was 25° C. Progress curves of the transfer of the farnesylpyrophosphate to the Dansyl-GCVLS substrate are shown in FIG. 79, Panel A and relative rates of the progress curve were determined and are shown in FIG. 79, Panel B.

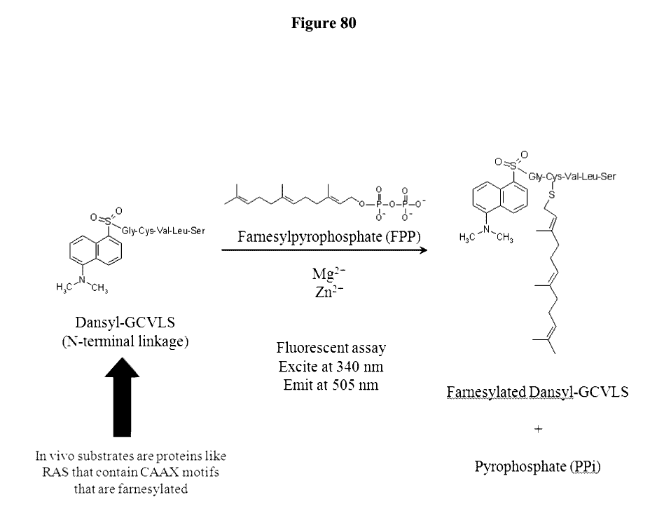

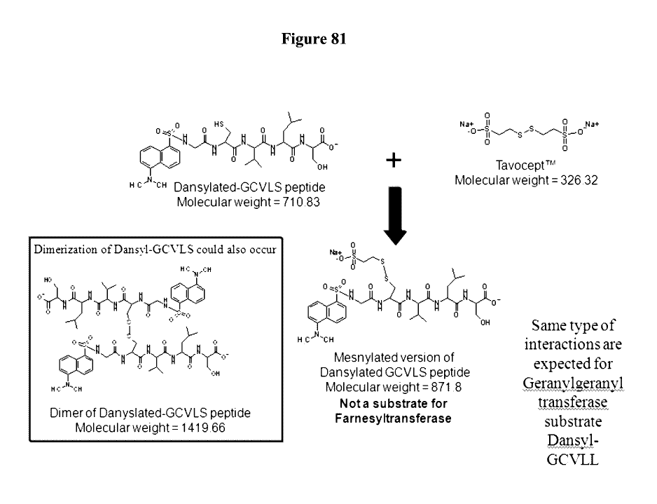

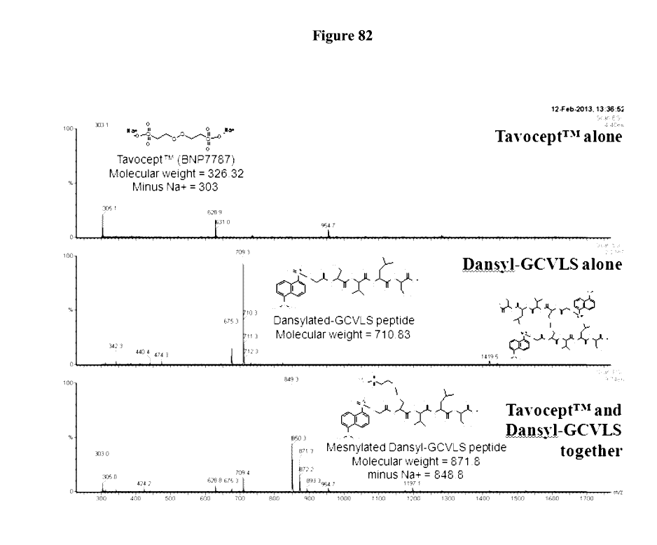

An overview of the FTase assay is shown in FIG. 80. It is postulated that Tavocept possesses the ability to directly modify FTase or the Dansyl-GCVLS peptide. Mass Spectroscopy experiments clearly indicate that Tavocept readily modified the danysl-GCVLS peptide yielding a Tavocept-mediated, xenobiotically modified Dansyl-GCVLS peptide that was not a substrate for FTase. See, FIG. 81 and FIG. 82.

### Summary of Studies on Human FTase and Tavocept Interactions

Tavocept inhibits farnesylation in a concentration-dependent manner.
Tavocept likely mediates inhibition of FTase activity by several mechanisms involving the covalent modification of the cysteine residue on the substrate peptide, Dansyl-GCVLS, and/or covalent modification of one or more of the cysteine residues on FTase.
Mass spectroscopy studies confirm that Tavocept rapidly xenobiotically modifies the Dansyl-GCVLS substrate peptide forming a covalent mixed-disulfide on Dansyl-GCVLS at cysteine.

VI. Conclusory Discussion

A. Tavocept is an Amino Acid-Specific Agent that Xenobiotically Modifies Multiple Target Molecules that can Cause Impaired Function and/or Direct Inhibition

As previously discussed, Tavocept (BNP7787) is a novel agent that has been evaluated in the clinic in patients with non-small cell lung cancer (NSCLC). Disclosed herein are numerous examples where Tavocept reacts with and forms mixed disulfides with protein cysteine residues, yielding covalently-bound, Tavocept-derived adducts on these target molecules. This process is referred to herein as Tavocept-mediated xenobiotic modification, and has been observed and characterized in a variety of proteins important in cellular growth and proliferation including, but not limited to, (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, and farnesyltransferase. As a non-limiting example, data on the crystal structure of ALK in complex with Tavocept-derived mesna adducts at 2.1 Å resolution established that Tavocept-derived mesna adducts were found at Cys1156 and Cys1235. Both adducts are relatively solvent exposed, although the adduct at Cys1156 clearly disrupts the orientations of the P-loop by sterically blocking the typically observed binding site for Phel 127. Because the P-loop binds the ATP-substrate phosphate groups, this P-loop disruption may alter the kinase activity of ALK or inhibitory potency of its small molecule inhibitors.

The amino acid-specific, multiple molecule-targeting nature of Tavocet's effect is important due to the fact that, except for a few types of cancer (e.g., chronic myelogenous leukemia (CML)), tumor cells are known to be genomically heterogeneous and contain subpopulations of cancer cells that often express different tumor-promoting proteins or that have multiple dysregulated, distinct but key pathways that modulate cell proliferation. Thus, it is hypothesized that Tavocept-mediated xenobiotic modification represents a novel mechanism of action for a therapeutic agent, as amino acid-specific modification (e.g., post-translational modification(s) of cysteine residue(s) in proteins) is a mechanism that can regulate a variety of cellular processes (e.g., glutathionylation, nitrosylation, prenylation, and palmitoylation). A number of cysteine-specific, multi-targeted mechanisms of action are summarized in Table 26, below.

TABLE 26
Examples of Cysteine-Specific Protein Modifications

| | | Protein Cofactor(s) |
|---|---|---|
| Modification | Specificity | Required? |
| Tavocept-mediated | Cysteines near or in alpha helices, with nearby residues | No |
| Xenobiotic | to stabilize the cysteinyl thiolate (BNPI unpublished | |
| modification | data) | |
| Glutathionylation | May involve cysteines with altered pKa's | Can be autocatalytic |
| | (vicinal to lysine, arginine or histidine) | or protein catalyzed |
| Nitrosylation | Possible specificity at the tertiary environment level | No |
| | around cysteine | |
| Prenylation | Varied sequences around target cysteine with a CaaX | Yes |
| (Farnesylation, | motif (a = aliphatic amino acid; X = one of several | |
| geranylgeranylation) | amino acids depending on protein) | |
| Palmitoylation | Varied Sequences | Can be autocatalytic |
| | | or protein catalyzed |

As discussed previously, Tavocept appears to enhance antitumor activity of cancer treating agents through cysteine-specific, multi-targeted mechanisms of action including those summarized in Table 27, below.

TABLE 27
Cellular Mechanisms of Tavocept's Cysteine-Specific, Multi-Targeted Effect

| Cellular Target of | Cellular consequence of BNP7787-modification |
|---|---|
| BNP7787 | and/or modulation |
| Cellular thiol/disulfide | BNP7787 and BNP7787-derived mesna disulfide heteroconjugates |
| balance | are pharmacological surrogate/modulators of physiological thiols |
| | and disulfides (e.g., glutathione, cysteine, and homocysteine) |
| γ-Glutamyltranspeptidase | BNP7787 and BNP7787-derived mesna disulfide heteroconjugates |
| Aminopeptidase N | can inhibit γ-glutamyltranspeptidase and aminopeptidase N enzyme |
| | activity |
| Tubulin | BNP7787 exerts direct and indirect protective interactions with |
| | tubulin |
| Anaplastic Lymphoma | BNP7787 disrupts/blocks ATP binding site resulting in inhibition |
| Kinase (ALK) | of ALK kinase activity (vide infra) |
| Mesenchymal Epithelial | Modification of non-active site cysteine(s) resulting in enzyme |
| Transition (MET) Factor | inhibition (MET). |
| Kinase | |
| ROS1 kinase | BNP7787 xenobiotically modifies ROS1 kinase in a time |
| | dependent manner |
| Redox Balance | BNP7787 and BNP7787-derived mesna disulfide heteroconjugates |
| | assisting in the maintenance of cellular redox balance and |
| | supporting cellular defenses against oxidative insult |
| Thioredoxin (Trx) | BNP7787 modifies non-catalytic cysteines important in redox |
| Glutaredoxin (Grx) | protein function/structure (Grx and Trx) |
| Thioredoxin (Trx) | BNP7787 and/or BNP7787-derived mesna disulfide |
| Glutaredoxin (Grx) | heteroconjugates function as alternative substrates/inhibitors (Trx, |
| | Grx) resulting in impaired enzyme activity |
| Peroxiredoxin (Prx) | BNP7787 disrupts active site structure (Prx) resulting in impaired |
| | enzyme activity |

Tavocept is expected to remain predominantly in the disulfide form in the plasma; (see, e.g., Hausheer F H, Parker A R, Petluru P N, et al. Mechanistic study of BNP7787-mediated cisplatin nephroprotection: modulation of human aminopeptidase N. Cancer Chemother. Pharmacol. 67(2):381-391 (2011)); however, the intracellular environment and the interstitial space are likely venues for Tavocept metabolism to mesna, mesna-disulfide heteroconjugates, and free thiols. Any of these species, Tavocept, Tavocept-derived mesna-disulfide heteroconjugates or intracellularly generated Tavocept-derived mesna, may modify proteins in vivo. The metabolism of Tavocept to mesna-disulfide heteroconjugates has been observed in in vitro studies and is supported by computational studies on non-enzymatic thiol transfer reactions involving physiological free thiols with Tavocept.

B. Tavocept Enhancement of Cancer Treating Agent Activity

Tavocept, Tavocept-derived mesna, and Tavocept-derived heteroconjugates may act via several possible routes to increase the cancer fighting activity of cancer treating agents. Studies have shown that Tavocept and the Tavocept metabolite, mesna, deplete the plasma thiols glutathione, cysteine and homocysteine and, while levels of thiols are low in the plasma, this effect may enhance the antitumor activity of multiple cancer treating agents. Additionally, the metabolism of Tavocept to mesna-disulfide heteroconjugates via thiol disulfide exchange reactions between Tavocept and glutathione, cysteine, and homocysteine are likely to be important in mediating Tavocept enhancement of antitumor activity through direct and indirect effects on proteins important in regulating the intracellular redox balance. The cellular redox environment is thought to be very important in determining whether or not a cell proliferates or undergoes apoptosis. Furthermore, although it is reported to have no cytotoxic effects, the Applicants have observed that pharmacologically relevant concentrations of mesna (≦400 μM) can be cytotoxic to some cell lines in vitro (unpublished data) and Tavocept is reduced to mesna intracellularly, thus this is another avenue through which Tavocept may enhance antitumor activity.

By way of non-limiting example, the studies disclosed herein indicate that Tavocept can directly inhibit ROS1 kinase in a time-dependent manner. ROS1 kinase and various other proteins that Tavocept modulates are important in cell proliferation in a number of cancers, including non-small cell lung cancer (NSCLC). Furthermore, as disclosed herein, various other human proteins including, but not limited to, (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, and farnesyltransferase are also modified and/or modulated by Tavocept. The specific effect of Tavocept on these aforementioned protein targets depends upon how the targeted cysteine residue(s) impacts the protein function and/or structure.

In summary, Tavocept is a cysteine-specific, multi-targeted modifier and/or modulator of protein function. Tavocept mediates the non-enzymatic xenobiotic modification of cysteine residues on these protein targets. By way of non-limiting example, experimental data disclosed herein provides evidence for the direct inhibition of ROS1 kinase by Tavocept and also indicates that Tavocept used in combination with the ATP-competitive inhibitor Crizotinib, results in potentiation of Crizotinib inhibition when Tavocept is incubated with ROS1 kinase prior to initiation of the kinase assays. As a xenobiotic, non-naturally occurring agent, Tavocept is autocatalytic and requires no protein co-factor to xenobiotically modify cysteine, but appears to be specific for cysteine residues located within a specific structural context (i.e., not all cysteine residues within a protein are modified). Tavocept-mediated xenobiotic modification represents a novel mechanism of action for a therapeutic agent and the Applicants hypothesize that the survival benefits seen in NSCLC (adenocarcinoma sub-type) patients may be a result of cysteine-specific, Tavocept-mediated xenobiotic modification, with the subsequent functional modification/modulation of one or more protein targets that are dysregulated in these patients.

VII. Summary of Data from Tavocept Phase III Clinical Trial

The Tavocept Phase III Clinical Trial was designed as a randomized, multi-center, double-blind, placebo-controlled trial of Tavocept in patients with incurable Stage IV primary adenocarcinoma of the lung treated with the standard chemotherapy drugs docetaxel or paclitaxel in combination with cisplatin administered every three weeks for up to 6 treatment cycles. In the Tavocept Phase III Clinical Trial pre-specification was made through stratification factors including sex (i.e., male versus female) and taxane treatment (i.e., paclitaxel versus docetaxel). Eligible patients had not received prior drug treatment for their cancer, and inclusion in the trial was also allowed for patients who had relapsed following surgery for earlier stage disease as well as patients with central nervous system metastasis. A total of 540 patients were enrolled on the trial.

The primary endpoint of this study was overall survival (OS); defined as the time period from the date of patient randomization to the date of death due to any cause. Additionally, the trial evaluated Tavocept's ability to concurrently prevent and mitigate common chemotherapy-induced toxicities.

Patients underwent procedures throughout three (3) defined periods in this study, which will be discussed in detail below.

Period I (Screening and Randomization):

Patient eligibility was determined by compliance with protocol-specified inclusion and exclusion criteria. Patients who reviewed and signed the informed consent, and successfully completed the screening process were randomized in a 1:1 ratio to receive Tavocept (Group A) or placebo (Group B).

Period II (Study Treatment):

Patients received standard combination chemotherapy (paclitaxel or docetaxel plus cisplatin) and either Tavocept or placebo once every 3 weeks for a maximum of 6 cycles, so long as they continued to have evidence of clinical benefit in the form of complete response, partial response, or stable disease, and were not experiencing unacceptable treatment-related toxicity or prolonged (>2 weeks from a scheduled treatment cycle) treatment delays.

Period III (Follow-Up for Progression and Survival):

All patients were followed for progression and survival.

If patients went off study for radiographic documented disease progression, such patients were followed for survival and for dates and types of any subsequent lines of treatment.

Patients who discontinued from the study without experiencing disease progression and who were not treated with additional subsequent therapy, continued to undergo repeat CT scans every 6 to 8 weeks for up to 6 months after going off study until they experienced either: (i) disease progression; or (ii) initiation of any subsequent line of therapy.

For patients who went off study without experiencing disease progression and were treated with subsequent lines of therapy, CT scans or bone scans or CNS MRI that may document any subsequent disease progression were completed prior to the initiation of any second-line therapy. Patients were followed for survival, and dates and type of any subsequent line(s) of additional post-study therapy by telephone and letter confirmation every 3 months for up to 2 years from the date of randomization, and then every 6 months for up to one year (total follow-up period of up to 3 years). Follow-up assessments were made to document the date and composition of subsequent line(s) of treatment, including chemotherapy, radiation therapy, or other forms of therapy.

A study flow diagram for the 3 periods of this Clinical Study is presented in Table 28, below.

### A. Treatments Administered

All patients received standard combination chemotherapy (taxane agent plus cisplatin) and either Tavocept or placebo. Patients were randomized in a 1:1 ratio to one of the two treatment groups as presented in Table 29, below.

TABLE 29

| | Group A (Tavocept Treatment Arm) | Group B (Placebo Arm) |
|---|---|---|
| Agents & | Patients received either: | Patients received either: |
| Administration | Paclitaxel 200 mg/m2 IV over 3 hours, followed by | Paclitaxel 200 mg/m2 IV over 3 hours, followed by |
| Schedule | Tavocept 18.4 g/m2 IV over 30 minutes, followed | placebo (0.9% NaCl) IV over 30 minutes, followed |
| | by cisplatin 80 mg/m2 IV over 30 to 60 minutes | by cisplatin 80 mg/m2 IV over 30 to 60 minutes |
| | OR | OR |
| | Docetaxel 75 mg/m2 IV over one hour, followed by | Docetaxel 75 mg/m2 IV over one hour, followed by |
| | Tavocept 18.4 g/m2 IV over 30 minutes, followed | placebo (0.9% NaCl) IV over 30 minutes, followed |
| | by cisplatin 80 mg/m2 IV over 30 to 60 minutes | by cisplatin 80 mg/m2 IV over 30 to 60 minutes |

Taxane treatment included required pre-medications and prophylactic anti-emetic regimens;
cisplatin treatment will include required prophylactic saline hydration and diuresis.

Treatment was administered every 3 weeks for a maximum of 6 cycles (one treatment cycle=21 days).

The amount of study drug was calculated based on the patient's body surface area (BSA). The calculated amount of study drug was rounded to the nearest 0.1 mg/m² (i.e., a value of “0.05” was rounded up to “0.1”).

### B. Primary Endpoint and Analysis

The primary endpoint for analysis was the duration of overall survival, defined as the time from the date of randomization to the date of death due to any cause. The number of mortality events used for the final analysis depended upon the number of patients enrolled into the study (provided the sample size was adjusted at the interim analysis). For the primary analysis, the expected number of total mortality events was approximately 416 mortality events for a sample size of 575 patients.

The one-sided log-rank test was performed in order to allow the application of the adaptive methodology. Details of this 2-stage adaptive design are described below. Kaplan-Meier survival curves (including identification of censored observations) was presented by treatment group, as well as median survival times and their 95% confidence intervals.

### C. Secondary Endpoint and Analysis

The following specific endpoints were evaluated with pre-specified analyses in order to evaluate for potential clinically and statistically significant differences between Tavocept- and placebo-treated patients. These endpoints were analyzed in the order identified below, and if medically and statistically significant outcomes were observed in this study, these endpoints were relevant to the clinical utility, administration, and labeling of Tavocept.
Progression-Free Survival (PFS): defined as the time from the date of randomization to date of first documented tumor/disease progression using RECIST or death due to any cause.
Incidence of a 30% or greater decrease in the calculated creatinine clearance relative to baseline calculated creatinine clearance.
Incidence of NCI-CTCAE grade 2, 3, or 4 anemia (hemoglobin).
Proportion of patients having no impact of chemotherapy-induced emesis on daily life as measured by the Functional Living Index-Emesis (no impact on daily life is defined as an average score of >6 on the seven-point scale).
Quality of life as measured by the FACT-L

PFS was summarized using Kaplan-Meier survival estimation procedures, and homogeneity of the treatment groups assessed using a log-rank test. Censored observations and 95% confidence intervals for the estimated median times was estimated. Patients without disease progression or death at the time of data cutoff were censored at the time of their last tumor assessment, even if such patients received subsequent lines of therapy. A preplanned sensitivity analysis was performed to compensate for the possible confounding effects of non-protocol therapy that was administered to patients who went off study prior to disease progression. Patients who went off study without disease progression prior to the initiation of any non-protocol therapy were censored at the last tumor assessment prior to the initiation date of non-protocol therapy.

The incidence of cisplatin renal toxicity and anemia, and the impact of emesis on daily life were analyzed using the Cochran-Mantel-Haenszel (CMH) tests for proportions. The CMH test was applied in the comparison between Tavocept and placebo where adjustment for prognostic factors is clinically important (e.g., adjusting for baseline characteristics). To control for Type I error due to multiple comparisons of safety data, a nominal, 2-sided p-value of less than 0.0125 was used for each comparison of renal toxicity, anemia, and emesis.

The FACT-L (version 4.0) was used to measure changes from baseline to end of study, and was summarized using continuous descriptive statistics by treatment group. Sign-rank and rank sum tests were used to compare within-group and between-group changes from baseline, respectively.

### D. Summary of Tavocept Phase III Clinical Trial Results

The results reported below are from an Interim Analysis of the Tavocept Phase III Trial. As described above, patients participating in the Tavocept Phase III Trial had adenocarcinoma of the lung. For purposes of the discussion below, references to “placebo” refer to the placebo arm of the Tavocept Phase III Trial, where patients received either paclitaxel or docetaxel and cisplatin plus placebo as described in Table 29 above.
Top line results from the Phase III clinical trial indicate a greater than 2 month overall median survival advantage in favor of Tavocept in subjects with advanced primary adenocarcinoma of the lung receiving a standard chemotherapy regimen of paxlitaxel or docetaxel plus cisplatin together with Tavocept or placebo (P-value=0.723 in favor of Tavocept and P-value=0.295 in favor of Tavocept excluding patients who received subsequent therapy after first-line).
Median survival advantage of 11.8 months in favor of Tavocept was observed in Females receiving paclitaxel and cisplatin (P-value=0.048; Hazard Ration (HR)=0.579). Tavocept median survival was 25.0 months versus 13.2 months for Placebo.
Median survival advantage of 13.6 months in favor of Tavocept in Female Non-Smokers receiving paclitaxel and cisplatin (P-value=0.017; Hazard Ratio (HR)=0.367). Tavocept median survival was 27.0 months versus 13.4 months for Placebo.
The 2-year survival for female non-smokers receiving paclitaxel and cisplatin was more than double for the Tavocept arm compared with the placebo arm (72.4% versus 32.3%). In addition, the 2-year survival for male and female non-smokers receiving paclitaxel and cisplatin was 63% for the Tavocept arm compared with 28% for the placebo arm.
The 2-year survival for all females receiving paclitaxel and cisplatin was 51% for the Tavocept arm compared with 31% for the placebo arm. In addition, the 2-year survival for all subjects receiving paclitaxel and cisplatin was 30% for the Tavocept arm compared with 25% for the placebo arm.
Median survival advantage of 12 months in favor of Tavocept in Male & Female Non-Smokers receiving paclitaxel and cisplatin (P-value=0.046; HR=0.519). Tavocept median survival was 25.2 months versus 13.2 months for Placebo.
Median survival advantage of almost 3 months (2.82 months) in favor of the Tavocept arm in PS 1 ECOG Performance Status subjects (P-value=0.3647; HR=0.898).
Median survival advantage of 11.1 months in favor of the Tavocept arm in PS 1 ECOG Performance Status females receiving paclitaxel and cisplatin (P-value=0.346; HR=0.526).
Median survival advantage of 4.0 months in favor of the Tavocept arm in PS 1 ECOG Performance Status males and females receiving paclitaxel and cisplatin (P-value=0.1162; HR=0.761).
Median survival advantage of 3 months in favor of the Tavocept arm in subjects greater than or equal to 65 years of age (P-value=0.9204; HR=0.977). This is an important finding, as a recent clinical trial found that Avastin (bevacizumab) did not confer a survival advantage in non-small cell lung cancer patients of the aforementioned age bracket who were treated with carboplatin and paclitaxel chemotherapy. See, Schrage, D., et al., Adding Avastin did not improve standard chemotherapy regimen in non-small cell lung cancer. Clin. Cancer Lett. 35(4):4 (2012).
Median survival advantage of 2.1 months in favor of the Tavocept arm in female subjects greater than or equal to 65 years of age receiving paclitaxel and cisplatin (P-value=0.4574; HR=0.621).
Median survival advantage of 3.5 months in favor of the Tavocept arm in male and female subjects greater than or equal to 65 years of age receiving paclitaxel and cisplatin (P-value=0.7042; HR=0.878).
Median survival advantage of almost 4 months (3.68 months) in favor of the Tavocept arm in subjects who received paclitaxel and cisplatin as the chemotherapeutic agent, instead of docetaxel and cisplatin (P-value=0.1822; HR=0.811). Tavocept median survival was 15.4 months versus 11.7 months for Placebo.
Median survival advantage in favor of Tavocept in Stage IV M1b subjects, with 11.33 months median survival for the Tavocept arm versus 9.79 months median survival for the Placebo arm (P-value=0.6118; HR=0.936).
Median survival advantage in favor of Tavocept in subjects that were Newly Diagnosed at the time of enrollment on the trial, with 14.19 months median survival in the Tavocept arm versus 12.16 months median survival in the Placebo arm (P-value=0.3308; HR=0.894).
Median survival advantage in favor of Tavocept in female subjects receiving paclitaxel plus cisplatin who were Newly Diagnosed at the time of enrollment on the trial, with 25.0 months median survival in the Tavocept arm versus 13.4 months median survival in the Placebo arm (P-value=0.0571; HR=0.572).
Median survival advantage in favor of Tavocept in male and female subjects receiving paclitaxel plus cisplatin who were Newly Diagnosed at the time of enrollment on the trial, with 15.3 months median survival in the Tavocept arm versus 12.2 months median survival in the Placebo arm (P-value=0.1362; HR=0.785).
Median survival advantage in favor of Tavocept in female subjects receiving paclitaxel plus cisplatin who did not receive any subsequent therapy following their participation in the Tavocept Phase III Trial, with 14.2 months median survival for the Tavocept arm versus 9.2 months median survival in the Placebo arm (P-value=0.3463; HR=0.693).
Median survival advantage in favor of Tavocept in male and female subjects receiving paclitaxel plus cisplatin who did not receive any subsequent therapy following their participation in the Tavocept Phase III Trial, with 10.0 months median survival for the Tavocept arm versus 8.3 months median survival in the Placebo arm (P-value=0.3364; HR=0.822).
Median survival advantage in favor of Tavocept in subjects participating in the trial who had CNS Metastases present, with 11.76 months median survival in the Tavocept arm versus 9.79 months median survival in the Placebo arm (P-value=0.5569; HR=0.768).
Survival advantage in favor of Tavocept in male and female subjects who had CNS Metastases present and received paclitaxel plus cisplatin in the trial, with a hazard ratio equal to 0.744 in favor of Tavocept (P-value=0.6473). Survival advantage in favor of Tavocept in female subjects who had CNS Metastases present and received paclitaxel plus cisplatin in the trial (P-value=0.0343).
Key pharmacological and biological mechanisms have been elucidated (as disclosed herein) that support Tavocept's role in these observed treatment benefits.
In the Tavocept Phase III Trial, the median period of additional survival after starting new chemotherapy for patients previously receiving Tavocept was 3.4 months longer than the comparative period of additional survival for placebo-treated patients. From the point of randomization on the study, patients receiving Tavocept and post-study new chemotherapy had a median overall survival of 23.0 months compared to a median overall survival of 19.6 months for patients receiving placebo and post-study new chemotherapy. Both of the above groups also concurrently received paclitaxel and cisplatin. The relative improvement in overall survival in Tavocept-treated patients compared to placebo-treated patients receiving post study therapy (12.2 versus 9.2 months, respectively) was over 50% greater than the improvement compared with patients who did not receive post study therapy (10.0 versus 8.3 months, respectively). In other words, the relative benefit observed for Tavocept appears to continue even after cessation of treatment. These observations provide further support for the view that the sulfur-containing, amino acid-specific small molecules of the present invention have the ability to condition the cellular environment for improved responses and outcomes in patients receiving other cancer treating agents even after cessation of treatment with such sulfur-containing, amino acid-specific small molecules. These observations also provide further support for the use of the sulfur-containing, amino acid-specific small molecules of the present invention (i) to improve the performance of other cancer treating agents and therapies (even after treatment with such sulfur-containing, amino acid-specific small molecules), and (ii) to provide benefit in maintenance therapy or adjuvant therapy regimens or settings with or without other cancer treating agents.
In addition, a comparison of the Kaplan Meier survival curves for the Tavocept Phase III Trial comparing the overall survival curve for the Tavocept arm of the trial compared to the overall survival curve for the Placebo arm of the trial indicates a flattening of the slope of the survival curve for the Tavocept arm (reduction in downward slope) relative to the slope of the Placebo arm during the period of administration of up to 6 cycles of Tavocept treatment. This observation provides additional support for the use of the sulfur-containing, amino acid-specific small molecules of the present invention to provide benefit in maintenance therapy or adjuvant therapy regimens and/or settings with or without other cancer treating agents.
The Tavocept arm in the Phase III trial also demonstrated important safety/toxicity profile advantages in terms of protection against chemotherapy-induced kidney toxicity, with an improvement in the relative creatine decrease from baseline in favor of Tavocept compared to placebo (P-value=0.0528) and reduced anemia in the Tavocept arm of the trial compared to the placebo arm.
Non-smokers with lung cancer represent an important patient population, as the overall incidence is large and growing. Moreover, other treatments for these patients have not demonstrated any substantial survival increases.
A high percentage of adenocarcinoma patients are either EGFR mutants or Met/ALK positive.
Lung cancer in non-smokers appears to affect females disproportionately compared with males. It is estimated that over 50% of the non-smoking population is female.
Met/ALK & EGFR WT are more common in non-smokers, who are most commonly female and present with advanced stage adenocarcinoma.

The results reported below are from an Interim Analysis of the Tavocept Phase III Clinical Trial. As described above, patients participating in the Tavocept Phase III Trial had adenocarcinoma of the lung. For purposes of the discussion below, references to “placebo” refer to the placebo arm of the Tavocept Phase III Trial, where patients received either paclitaxel or docetaxel and cisplatin plus placebo as described in Table 29 above.

All patents, publications, scientific articles, web sites, and the like, as well as other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The present invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”. The letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A method for the contemporaneous, heterogeneously-oriented metabolic modification and/or modulation of the expression level of multiple target molecules; wherein any combination of target molecules is selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; and
wherein said method is comprised of the administration of the sulfur-containing, amino acid-specific small molecules of the present invention in an amount sufficient to provide a therapeutic benefit to a subject suffering from one or more types of cellular metabolic anomalies or other pathophysiological conditions where there is evidence of abnormal expression levels of one or more of said target molecules and cellular metabolic modification and/or modulation of the target molecule(s) is used to treat said subject suffering from one or more cellular metabolic anomalies or other pathophysiological conditions.

2. A method for the contemporaneous, heterogeneously-oriented metabolic modification and/or modulation of the biochemical activity of multiple target molecules; wherein any combination of target molecules is selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; and
wherein said method is comprised of the administration of the sulfur-containing, amino acid-specific small molecules of the present invention in an amount sufficient to provide a therapeutic benefit to a subject suffering from one or more types of cellular metabolic anomalies or other pathophysiological conditions where there is evidence of the biochemical activities of said multiple target molecules being abnormal and the cellular metabolic modification and/or modulation of the target molecule(s) is used to treat said subject suffering from one or more cellular metabolic anomalies or other pathophysiological conditions.

3. A method for quantitatively ascertaining: (i) the level of expression of DNA, mRNA, or protein, and/or (ii) the abnormal biochemical activities of any combination of multiple target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, in cells which have been isolated from a subject suffering from one or more types of cellular metabolic anomalies or other pathophysiological conditions where there is evidence of: (i) elevated levels of expression; and/or (ii) abnormal biochemical activities of any combination of said multiple target molecules;
wherein said method for quantitatively ascertaining: (i) the level of expression of DNA, mRNA, or protein, and/or (ii) the abnormal biochemical activities of any combination of said multiple target molecules is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

4. The method of claim 1 or claim 2, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 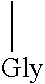
pharmaceutically-acceptable salts thereof.

5. The method of claim 4, wherein said sulfur-containing, amino acid-specific small molecule is disodium 2,2′-dithio-bis-ethane sulfonate.

6. The method of claim 1 or claim 2, wherein said cellular metabolic anomalies or other pathophysiological conditions for treatment with sulfur-containing, amino acid-specific small molecules of the present invention are cancers selected from the group consisting of: colorectal cancer, brain cancer and cancer of the Central Nervous System, gastric cancer, esophageal cancer, cancer of the biliary tract, gallbladder cancer, breast cancer, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

7. The method of claim 1 or claim 2, wherein said cellular metabolic anomalies or other pathophysiological conditions for treatment with sulfur-containing, amino acid-specific small molecules of the present invention are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigmentosum, Cockayne syndrome, Fanconi anemia, and cerebro-oculo-facio-skeletal syndrome.

8. The method of claim 1 or claim 2, further comprising the administration of one or more cancer treating agents in combination with the sulfur-containing, amino acid-specific small molecules of the present invention; wherein, said cancer treating agents are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; and various other cytotoxic and cytostatic agents.

9. The method of claim 1 or claim 2, further comprising the administration of the sulfur-containing, amino acid-specific small molecules of the present invention in combination with one or more of the following medicaments, including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

10. A contemporaneous, heterogeneously-oriented method to metabolically modify and/or modulate the intracellular environment of cancer cells in a subject suffering from one or more types of cancer such that the intracellular environment of said cancer cells is made more amenable to the pharmacological activity of the one or more chemotherapeutic, cytotoxic, or cytostatic agent(s) administered to treat the subject's cancer; wherein said method is comprised of the administration of an amount of the sulfur-containing, amino acid-specific small molecules of the present invention sufficient to metabolically modify and/or modulate the intracellular environment of cancer cells in said subject suffering from one or more types of cancer; and
wherein said cancer exhibits evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif.

11. A contemporaneous, heterogeneously-oriented method to metabolically modify and/or modulate the intracellular environment of cells in a subject suffering from one or more types of non-cancerous, cellular metabolic anomalies or other pathophysiological conditions such that the intracellular environment of said cells is made more amenable to the pharmacological activity of one or more medicinal agent(s) administered to treat the subject's non-cancerous, cellular metabolic anomalies or other pathophysiological conditions; wherein said method is comprised of the administration of an amount of the sulfur-containing, amino acid-specific small molecules of the present invention sufficient to metabolically modify and/or modulate the intracellular environment of cells in said subject suffering from one or more types of non-cancerous, cellular metabolic anomalies or other pathophysiological conditions; and
wherein said non-cancerous, cellular metabolic anomalies or other pathophysiological conditions exhibit evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif.

12. A method for quantitatively ascertaining: (i) the level of expression of DNA, mRNA, or protein, and/or (ii) the abnormal biochemical activities of any combination of multiple target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, in cells which have been isolated from a subject suffering from one or more types of cancer or one or more types of non-cancerous, cellular metabolic anomalies or other pathophysiological conditions where there is evidence of: (i) abnormal levels of expression; and/or (ii) abnormal biochemical activities of any combination of said multiple target molecules;
wherein said method for quantitatively ascertaining: (i) the abnormal level of expression of DNA, mRNA, or protein, and/or (ii) the abnormal biochemical activities of any combination of said multiple target molecules is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

13. The method of claim 11 or claim 12, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 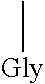
pharmaceutically-acceptable salts thereof.

14. The method of claim 13, wherein said sulfur-containing, amino acid-specific small molecule is disodium 2,2′-dithio-bis-ethane sulfonate.

15. The method of claim 10, wherein said cancer or cancers are selected from the group consisting of: colorectal cancer, gastric cancer, esophageal cancer, cancer of the biliary tract, gallbladder cancer, breast cancer, brain cancer and cancer of the Central Nervous System; cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

16. The method of claim 11, wherein said non-cancerous, cellular metabolic anomalies or other pathophysiological conditions for treatment with sulfur-containing, amino acid-specific small molecules of the present invention are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigementosum, Cockayne syndrome, Fanconi anemia, and cerebro-oculo-facio-skeletal syndrome.

17. The method of claim 10, further comprising the administration of one or more chemotherapeutic, cytotoxic, or cytostatic agent(s) in combination with the sulfur-containing, amino acid-specific small molecules of the present invention; wherein: said chemotherapeutic, cytotoxic, or cytostatic agent(s) are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; antivirals; and various other cytotoxic and cytostatic agents.

18. The method of claim 10 or claim 11, further comprising the administration of the sulfur-containing, amino acid-specific small molecules of the present invention in combination with one or more of the following medicaments, including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

19. A contemporaneous, heterogeneously-oriented method for treating a subject suffering from one or more types of cancer where a contemporaneous, heterogeneously-oriented, multiple targeted, molecular-directed treatment regimen is pharmacologically-effective in overcoming cellular metabolic resistance to treatment in a subject with one or more types of cancer; wherein such cellular metabolic resistance to treatment is associated with the cancer cells exhibiting evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; and wherein said method is comprised of the administration of an amount of the sulfur-containing, amino acid-specific small molecules of the present invention sufficient to overcome the cellular metabolic resistance to treatment in said subject with one or more types of cancer.

20. A contemporaneous, heterogeneously-oriented method for treating a subject suffering from one or more types of cellular metabolic anomalies or other pathophysiological conditions where a contemporaneous, heterogeneously-oriented multiple targeted, molecular-directed treatment regimen is pharmacologically-effective in overcoming cellular metabolic resistance to treatment in a subject with one or more types of cellular metabolic anomalies or other pathophysiological conditions; wherein such cellular metabolic resistance to treatment is associated with exhibiting evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; and wherein said method is comprised of the administration of an amount of the sulfur-containing, amino acid-specific small molecules of the present invention sufficient to overcome the cellular metabolic resistance to treatment in said subject with one or more types of cellular metabolic anomalies or other pathophysiological conditions.

21. A method for quantitatively ascertaining: (i) the level of expression of DNA, mRNA, or protein, and/or (ii) the abnormal biochemical activities of any combination of multiple target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, in cells which have been isolated from a subject suffering from one or more types of cancer or one or more types of non-cancerous, cellular metabolic anomalies or other pathophysiological conditions where there is evidence of: (i) abnormal levels of expression; and/or (ii) abnormal biochemical activities of any combination of said multiple target molecules;
wherein said method for quantitatively ascertaining: (i) the level of expression of DNA, mRNA, or protein, and/or (ii) the abnormal biochemical activities of any combination of said multiple target molecules is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

22. The method of claim 19 or claim 20, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 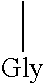
pharmaceutically-acceptable salts thereof.

23. The method of claim 22, wherein said sulfur-containing, amino acid-specific small molecule is disodium 2,2′-dithio-bis-ethane sulfonate.

24. The method of claim 19, wherein said cancer or cancers are selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

25. The method of claim 20, wherein said cellular metabolic anomalies or other pathophysiological conditions for treatment with sulfur-containing, amino acid-specific small molecules of the present invention are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigmentosum, Cockayne syndrome, Fanconi anemia, and cerebro-oculo-facio-skeletal syndrome.

26. The method of claim 19 or claim 20, further comprising the administration of one or more of the following medicaments, including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

27. A method to determine the dosage of the sulfur-containing, amino acid-specific small molecules of the present invention required to be administered to provide the maximal therapeutic benefit to a subject with one or more types of cancer that exhibit evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif;
wherein said method is comprised of quantitatively determining: (i) the abnormal biochemical activity and/or (ii) the abnormal expression of any combination of said target molecules and then using the results obtained to select the amount of the sulfur-containing, amino acid-specific small molecules of the present invention to administer to provide a therapeutic benefit to said subject in need thereof; and
wherein said method for quantitatively ascertaining the amount of the sulfur-containing, amino acid-specific small molecules of the present invention required to be administered to provide the maximal therapeutic benefit to a subject with one or more types of cancer that exhibit evidence of: (i) abnormal biochemical activity and/or (ii) abnormal expression of any combination of target molecules is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies. (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

28. A method to determine the dosage of the sulfur-containing, amino acid-specific small molecules of the present invention required to be administered to provide the maximal therapeutic benefit to a subject with one or more types of cellular metabolic anomalies or other undesirable physiological conditions that exhibit evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif;
wherein said method is comprised of quantitatively determining (i) the abnormal biochemical activity and/or (ii) the abnormal expression of any combination of said target molecules and then using the results obtained to select the amount of the sulfur-containing, amino acid-specific small molecules of the present invention to administer to provide a therapeutic benefit to said subject in need thereof; and
wherein said method for quantitatively ascertaining the amount of the sulfur-containing, amino acid-specific small molecules of the present invention required to be administered to provide the maximal therapeutic benefit to a subject with one or more types of cellular metabolic anomalies or other undesirable physiological conditions that exhibit evidence of: (i) abnormal biochemical activity and/or (ii) abnormal expression of any combination of target molecules selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies. (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

29. A method for quantitatively ascertaining the amount of the sulfur-containing, amino acid-specific small molecules of the present invention required to be administered to provide the maximal therapeutic benefit to a subject with: (a) one or more types of cancer or (b) one or more types of non-cancerous, cellular metabolic anomalies or other undesirable physiological conditions that exhibit evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, in cells which have been isolated from said subject with: (a) one or more types of cancer or (b) one or more types of non-cancerous, cellular metabolic anomalies or other pathophysiological conditions that exhibit evidence of: (i) abnormal biochemical activity; and/or (ii) abnormal expression of any combination of said multiple target molecules; of any combination of said target molecules and then using the results obtained to select the amount of the sulfur-containing, amino acid-specific small molecules of the present invention to administer to provide a therapeutic benefit to said subject in need thereof;
wherein said method for quantitatively ascertaining the amount of the sulfur-containing, amino acid-specific small molecules of the present invention required to be administered to provide the maximal therapeutic benefit to a subject with one or more types of cellular metabolic anomalies or other undesirable physiological conditions that exhibit evidence of: (i) abnormal biochemical activity and/or (ii) abnormal expression of any combination of target molecules selected from the group consisting of: and
wherein said method for quantitatively ascertaining the amount of the sulfur-containing, amino acid-specific small molecules of the present invention required to be administered to provide the maximal therapeutic benefit to a subject with (a) one or more types of cancer or (b) one or more types of non-cancerous, cellular metabolic anomalies or other pathophysiological conditions that exhibit evidence of: (i) abnormal biochemical activity; and/or (ii) abnormal expression of any combination of said multiple target molecules and then using the results obtained to select the amount of the sulfur-containing, amino acid-specific small molecules of the present invention to administer to provide a therapeutic benefit to said subject in need thereof; and
wherein said method for quantitatively ascertaining the amount of the sulfur-containing, amino acid-specific small molecules of the present invention required to be administered to provide the maximal therapeutic benefit to a subject with (a) one or more types of cancer or (b) one or more types of non-cancerous, cellular metabolic anomalies or other pathophysiological conditions that exhibit evidence of: (i) abnormal biochemical activity; and/or (ii) abnormal expression of any combination of said multiple target molecules to a subject with one or more types of cellular metabolic anomalies or other undesirable physiological conditions that exhibit evidence of: (i) abnormal biochemical activity and/or (ii) abnormal expression of any combination of target molecules selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies. (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

30. The method of claim 28 or claim 29, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 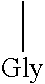
pharmaceutically-acceptable salts thereof.

31. The method of claim 30, wherein said sulfur-containing, amino acid-specific small molecule is disodium 2,2′-dithio-bis-ethane sulfonate.

32. The method of claim 28, wherein said cancers selected from the group consisting of: wherein said cancer or cancers are selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

33. The method of claim 29, wherein said cellular metabolic anomalies or other pathophysiological conditions for treatment with sulfur-containing, amino acid-specific small molecules of the present invention are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigementosum, Cockayne syndrome, Fanconi anemia, and cerebro-oculo-facio-skeletal syndrome.

34. The method of claim 28 or claim 29, further comprising the administration of one or more of the following medicaments in combination with the sulfur-containing, amino acid-specific small molecules of the present invention; comprising the administration of one or more of the following medicaments, which include: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

35. A method for use in: (a) the selection of subjects for treatment; (b) the determination of the most effective cancer treating agent(s) to be administered in combination with the administration of the sulfur-containing, amino acid-specific small molecules of the present invention; (c) the dosage of the cancer treating agent(s) to be administered; (d) the determination of the length and/or number of treatment cycles; (e) adjustment of the specific cancer treating agent(s) used and the dosage administered during treatment; and/or (f) ascertaining the potential treatment responsiveness of the specific cancer to the cancer treating agent(s) selected for administration to said subject having one or more types of cancer;
wherein said method is comprised of quantitatively determining the expression levels and/or the biochemical activity of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, and then using this expression level and/or biochemical activity data in determining: (i) the specific subjects to be treated; (ii) the cancer treating agent(s) to be administered in combination with the administration of the sulfur-containing, amino acid-specific small molecules of the present invention; (iii) the dosage of the cancer treating agent(s) to be administered; (iv) the length and/or number of cancer treating cycles to be administered; (v) the adjustment of the specific cancer treating agent(s) used and the dosages administered during the treatment regimen; and/or (vi) ascertaining the potential treatment responsiveness of the specific cancer to the cancer treating agents (s) selected to be administered to said subject having one or more types of cancer; and
wherein the method for quantitatively determining the dosage of the most effective chemotherapeutic agent(s) and the sulfur-containing, amino acid-specific small molecules of the present invention required to be administered to provide the maximal therapeutic benefit to a subject with one or more types of cancer that exhibits evidence of abnormal biochemical activity and/or abnormal expression of any combination of said multiple target molecules is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

36. The method of claim 35, wherein said cancers are selected from the group consisting of: wherein said cancers selected from the group consisting of: wherein said cancer or cancers are selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

37. The method of claim 35, wherein said cancer treating agents are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; and various other cytotoxic and cytostatic agents. fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; aziridine-containing compounds; and various other cytotoxic and cytostatic agents.

38. The method of claim 35, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 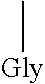
pharmaceutically-acceptable salts thereof.

39. The method of claim 38, wherein said sulfur-containing, amino acid-specific small molecule is disodium 2,2′-dithio-bis-ethane sulfonate.

40. The method of claim 35, further comprising the administration of one or more of the following cancer treating agents to be administered in combination with the administration of the sulfur-containing, amino acid-specific small molecules of the present invention; wherein said cancer treating agents include: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

41. The method of claim 35, wherein the subjects selected for treatment are further categorized for selection into one or more of the subgroups selected from the group consisting of: (i) female subjects; (ii) female, non-smoker subjects; (iii) female, non-smoker subjects with abnormal expression of anaplastic lymphoma kinase (ALK), mesenchymal epithelial transition (MET) kinase, and/or epidermal growth factor receptor (EGFR); (iv) male and female non-smoker subjects; (v) subjects over 65 years of age; (vi) female subjects over 65 years of age; (vii) newly diagnosed subjects; subjects with PS 1 in ECOG performance status; (viii) subjects who have central nervous system (CNS) metastases present; and (ix) subjects whose cancer has been categorized as Stage M1a/M1b.

42. A method for use in: (a) the selection of specific subjects for treatment; (b) the determination of the most effective medicinal agent(s) in combination with the administration of the sulfur-containing, amino acid-specific small molecules of the present invention; (c) the selection of the dosage of the medicinal agent(s) to be administered; (d) the determination of the length and/or number of treatment cycles to be administered; (e) adjustment of the specific medicinal agent(s) used and the dosages administered during treatment; and/or (f) ascertaining the potential treatment responsiveness of the specific disease to the medicinal agents(s) selected to be administered to a subject having one or more types of non-cancerous, cellular metabolic anomalies or other pathophysiological conditions;
wherein said method is comprised of quantitatively determining the expression levels and/or biochemical activity of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, and then using this expression level and/or biochemical activity data in determining: (i) the specific subjects to be treated; (ii) the medicinal agent(s) to be administered in combination with the administration of the sulfur-containing, amino acid-specific small molecules of the present invention; (iii) determining the dosage of the medicinal agent(s) to be administered; (iv) the length and/or number of treatment cycles to be administered; (v) the adjustment of the specific medicinal agent(s) administered and the dosages administered during treatment regimen; and/or (vi) ascertaining the potential treatment responsiveness of the specific disease to the medicinal agents (s) selected to be administered to said subject having one or more types of cellular metabolic anomalies or other pathophysiological conditions; and
wherein the method for quantitatively determining the dosages of the most effective medicinal agent(s) and the sulfur-containing, amino acid-specific small molecules of the present invention required to be administered to provide the maximal therapeutic benefit to said subject with one or more types of non-cancerous, cellular metabolic anomalies or other undesirable physiological conditions that exhibit evidence of abnormal biochemical activity and/or abnormal expression of any combination of said multiple target molecules is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

43. The method of claim 42, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 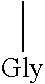
pharmaceutically-acceptable salts thereof.

44. The method of claim 43, wherein said sulfur-containing, amino acid-specific small molecule is disodium 2,2′-dithio-bis-ethane sulfonate.

45. The method of claim 42, wherein said cellular metabolic anomalies or other pathophysiological conditions for treatment with the present invention are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigementosum, Cockayne syndrome, Fanconi anemia, and cerebro-oculo-facio-skeletal syndrome.

46. The method of claim 42, further comprising the administration of one or more of the medicinal agent(s) to be administered in combination with the administration of the sulfur-containing, amino acid-specific small molecules of the present invention; wherein said medicinal agent(s) include: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

47. A contemporaneous, heterogeneously-oriented method for maximizing or extending the length of time before there is cancer progression in a subject who has one or more types of cancers that exhibit evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; wherein said method comprises the administration of the sulfur-containing, amino acid-specific small molecules of the present invention which function to delay the reoccurrence and/or progression of said cancer or cancers in the subject by modifying and/or modulating: (i) the abnormal biochemical activity and/or (ii) the abnormal expression of any combination of said target molecules.

48. The method of claim 46, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 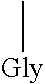
pharmaceutically-acceptable salts thereof.

49. The method of claim 47, wherein said sulfur-containing, amino acid-specific small molecule is disodium 2,2′-dithio-bis-ethane sulfonate.

50. The method of claim 46, wherein said cancers are selected from the group consisting of: wherein said cancers selected from the group consisting of: wherein said cancer or cancers are selected from the group consisting of: colorectal cancer, gastric cancer, esophageal cancer, cancer of the biliary tract, gallbladder cancer, breast cancer, cervical cancer, ovarian cancer, endometrial cancer, vaginal cancer, uterine cancer, prostate cancer, hepatic cancer, adenocarcinoma, pancreatic cancer, lung cancer, lymphoma, and cancers of the blood.

51. The method of claim 46, further comprising the administration of one or more cancer treating agents including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

52. A kit for use in the treatment of a subject having one or more cancers that are resistant to the cancer treating agent or agents being used to treat said subject with cancer, wherein said cancers are any cancer which exhibits evidence of: (i) abnormal biochemical activity and/or (ii) abnormal expression of one or more target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and/or other target protein (possessing a similar active site or structural motif)-mediated resistance to the chemotherapeutic agent or agents being used to treat said subject with cancer;
wherein said kit comprises: (a) one or more cancer treating agents; (b) the sulfur-containing, amino acid-specific small molecules of the present invention; and (c) instructions for administering said cancer treating agents and the sulfur-containing, amino acid-specific small molecules of the present invention to a subject with one or more types of cancer which are resistant to the chemotherapeutic agent or agents being used to treat said subject with cancer.

53. A kit for use in the treatment of a subject having one or more cancers that are resistant to the cancer treating agent or agents being used to treat said subject with cancer, wherein said cancers are any cancer which exhibit evidence of: (i) abnormal expression of and/or (ii) abnormal biochemical activity in the target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and/or other target proteins possessing a similar active site or structural motif-mediated resistance to the cancer treating agent or agents being used to treat said subject with cancer;
wherein said kit comprises: (a) one or more cancer treating agents; (b) the sulfur-containing, amino acid-specific small molecules of the present invention; and (c) instructions for administering said cancer treating agent(s) and the sulfur-containing, amino acid-specific small molecules of the present invention to a subject with one or more types of cancer which are resistant to the cancer treating agent or agents being used to treat said subject with cancer.

54. The kit of claim 51 or claim 52, wherein said cancers are selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

55. The kit of claim 51 or claim 52, wherein said cancer treating agent or agents are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; aziridine-containing compounds; and various other cytotoxic and cytostatic agents.

56. The kit of claim 51 or claim 52, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 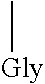
pharmaceutically-acceptable salts thereof.

57. The kit of claim 55, wherein said sulfur-containing, amino acid-specific small molecule is disodium 2,2′-dithio-bis-ethane sulfonate.

58. The kit of claim 51 or claim 52, wherein said kits further comprise the administration of one or more cancer treating agents including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

59. A cancer treating agent which modifies and/or modulates the expression levels and/or biochemical activity of one or more of the target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; wherein said cancer treating agent is the sulfur-containing, amino acid-specific small molecules of the present invention administered in an amount sufficient to provide a therapeutic benefit to a subject having one or more types of cancer which exhibit evidence of: (i) the abnormal expression level; and/or (ii) the abnormal biochemical activity of one or more of said target molecules; and wherein the abnormal expression level and/or the abnormal biochemical activity of said target molecules must be modified and/or modulated in order to treat said subject having one or more types of cancer.

60. A medicament which modifies and/or modulates the expression levels and/or biochemical activity of one or more of the target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; wherein said medicament is the sulfur-containing, amino acid-specific small molecules of the present invention administered in an amount sufficient to provide a therapeutic benefit to a subject having one or more types of cellular metabolic anomalies or other undesirable physiological conditions which exhibit evidence of: (i) the abnormal expression level; and/or (ii) the abnormal biochemical activity of one or more of said target molecules; and wherein the abnormal expression level and/or the abnormal biochemical activity of said target molecules must be modified and/or modulated in order to treat said subject having one or more cellular metabolic anomalies or other undesirable physiological conditions.

61. The cancer treating agent of claim 58, wherein said cancers are selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

62. The medicament of 59, wherein said cellular metabolic anomalies or other pathophysiological conditions for treatment with the present invention are non-cancer diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigementosum, Cockayne syndrome, Fanconi anemia, and cerebro-oculo-facio-skeletal syndrome.

63. The cancer treating agent of claim 58 or the medicament of claim 59, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 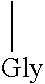
pharmaceutically-acceptable salts thereof.

64. The cancer treating agent of claim 58 or the medicament of claim 59, wherein said sulfur-containing, amino acid-specific small molecule is disodium 2,2′-dithio-bis-ethane sulfonate.

65. A method for the prophylactic use of the sulfur-containing, amino acid-specific small molecules of the present invention administered in an amount sufficient to provide a prophylactic benefit to a subject who has previously suffered from one or more types of cancers that exhibited evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; wherein the sulfur-containing, amino acid-specific small molecules of the present invention function to mitigate or prevent the reoccurrence of said cancer or cancers in said subject by modifying and/or modulating: (i) the abnormal biochemical activity and/or (ii) the abnormal expression of any combination of said target molecules.

66. The method of claim 64, wherein said cancers are selected from the group consisting of: wherein said cancers selected from the group consisting of: wherein said cancer or cancers are selected from the group consisting of: colorectal cancer, gastric cancer, esophageal cancer, cancer of the biliary tract, gallbladder cancer, breast cancer, brain cancer or cancer of Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

67. The method claim 64, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 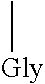
pharmaceutically-acceptable salts thereof.

68. The method of claim 66, wherein said sulfur-containing, amino acid-specific small molecule is disodium 2,2′-dithio-bis-ethane sulfonate.

69. The method of claim 64 which further comprises the administration of one or more cancer treating agents including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

70. A method for the prophylactic use of the sulfur-containing, amino acid-specific small molecules of the present invention administered in an amount sufficient to provide a prophylactic benefit to a subject who has previously suffered from one or more types of cellular metabolic anomalies or other undesirable physiological conditions that exhibited evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; wherein the sulfur-containing, amino acid-specific small molecules of the present invention function to mitigate or prevent the reoccurrence of said cellular metabolic anomalies or other undesirable physiological conditions in said subject by modifying and/or modulating: (i) the abnormal biochemical activity and/or (ii) the abnormal expression of any combination of said target molecules.

71. The method of claim 69, wherein said cellular metabolic anomalies or other pathophysiological conditions for treatment with the present invention are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigementosum, Cockayne syndrome, Fanconi anemia, and cerebro-oculo-facio-skeletal syndrome.

72. The method claim 68, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 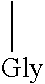
pharmaceutically-acceptable salts thereof.

73. The method of claim 71, wherein said sulfur-containing, amino acid-specific small molecule is disodium 2,2′-dithio-bis-ethane sulfonate.

74. The method of claim 68 which further comprises the administration of one or more medicaments including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

75. A method to restore normal cellular biochemical function and/or the normal expression level of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; wherein said method is comprised of the administration of the sulfur-containing, amino acid-specific small molecules of the present invention in an amount sufficient to provide a therapeutic benefit to a subject having one or more types of cancer which exhibit evidence of abnormal cellular biochemical functions and/or abnormal expression levels of said target molecules; and wherein said cellular biochemical fanctions and/or expression levels must be modified and/or modulated in order to treat said subject with cancer.

76. The method of claim 74, wherein said cancers selected from the group consisting of: colorectal cancer, gastric cancer, esophageal cancer, cancer of the biliary tract, gallbladder cancer, breast cancer, brain cancer and cancers of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

77. The method claim 74, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 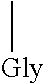
pharmaceutically-acceptable salts thereof.

78. The method of claim 76, wherein said sulfur-containing, amino acid-specific small molecule is disodium 2,2′-dithio-bis-ethane sulfonate.

79. The method of claim 74, which further comprises the administration of one or more cancer treating agents including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

80. A method to restore normal cellular biochemical function and/or the normal expression level of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; wherein said method is comprised of the administration of the sulfur-containing, amino acid-specific small molecules of the present invention in an amount sufficient to provide a therapeutic benefit to a subject having one or more types of cellular metabolic anomalies or other undesirable physiological conditions which exhibit evidence of abnormal cellular biochemical functions and/or abnormal expression levels of said target molecules; and wherein the abnormal cellular biochemical functions and/or abnormal expression levels of said target molecules must be modified and/or modulated in order to treat said subject with metabolic anomalies or other undesirable physiological conditions.

81. The method of claim 79, wherein said cellular metabolic anomalies or other pathophysiological conditions for treatment with the present invention are non-cancerous diseases selected from the group consisting of: heart failure, heart disease, hypertension, myocardial infarction, vascular disease, atherosclerosis, diabetes-induced heart disease, neurodegenerative diseases, Parkinson's disease, ALS, neurovascular dementia, autoimmune diseases, systemic lupus erythematosus, Graves orbitopathy, alcoholic liver disease, inflammatory bowel disease, cystic fibrosis, inflammatory diseases, diabetes, rheumatoid arthritis, progeria, Xeroderma pigmentosum, Cockayne syndrome, Fanconi anemia, and cerebro-oculo-facio-skeletal syndrome.

82. The method claim 79, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 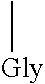
pharmaceutically-acceptable salts thereof.

83. The method of claim 81, wherein said sulfur-containing, amino acid-specific small molecule is disodium 2,2′-dithio-bis-ethane sulfonate.

84. The method of claim 79, which further comprises the administration of one or more medicaments including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

85. A method for the maintenance of a subject, who has one or more cancers, in a constant, steady physiological state such that said cancer(s) do not progress; wherein said method is comprised of the contemporaneous, heterogeneously-oriented metabolic modification and/or modulation of: (i) the expression level and/or (ii) the biochemical function of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif; and wherein the method is comprised of the administration of the sulfur-containing, amino acid-specific small molecules of the present invention in an amount sufficient to provide the maximal therapeutic benefit to a subject having one or more types of cancer which exhibit evidence of the expression level and/or biochemical function of one or more target molecules being abnormal; and wherein metabolic modification and/or modulation of the target molecule(s) exhibiting evidence of: (i) abnormal expression level and/or (ii) abnormal biochemical function is used to treat said subject in need thereof.

86. The method of claim 84, wherein said cancer is selected from the group consisting of: colorectal cancer, gastric cancer, esophageal cancer, cancer of the biliary tract, gallbladder cancer, breast cancer, brain cancer and cancer of the Central Nervous System, cervical cancer, ovarian cancer, endometrial cancer, vaginal cancer, uterine cancer, prostate cancer, hepatic cancer, adenocarcinoma, pancreatic cancer, lung cancer, myeloma, lymphoma, and cancers of the blood.

87. The method claim 84, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 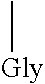
pharmaceutically-acceptable salts thereof.

88. The method of claim 86, wherein said sulfur-containing, amino acid-specific small molecule is disodium 2,2′-dithio-bis-ethane sulfonate.

89. The method of claim 84, which further comprises the administration of one or more additional medicaments including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (v) PD-1, PD-L1, and other checkpoint receptor inhibiting agents; (vi) immune checkpoint pathway modulatory antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

90. A contemporaneous, heterogeneously-oriented, target molecule-directed treatment method, wherein said method comprises the administration of one or more cancer treating agents and an amount of the sulfur-containing, amino acid-specific small molecules of the present invention sufficient to provide a therapeutic benefit to a subject with cancer which is selected from the group consisting of: acute lymphocytic leukemia (ALL), acute myelogenous leukemia (AML), or lymphoma; wherein said cancers exhibit evidence of: (i) abnormal biochemical activity and/or (ii) abnormal expression of the tyrosine kinase enzyme, anaplastic lymphoma kinase (ALK) and/or the epidermal growth factor receptor (EGFR).

91. The method of claim 89, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 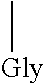
pharmaceutically-acceptable salts thereof.

92. The method of claim 90, wherein said sulfur-containing, amino acid-specific small molecule is disodium 2,2′-dithio-bis-ethane sulfonate.

93. The method of claim 89, wherein said cancer treating agent or agents are selected from the group consisting of: fluropyrimidines; pyrimidine nucleosides; purine nucleosides; anti-folates, platinum agents; anthracyclines/anthracenediones; epipodophyllotoxins; camptothecins; vinca alkaloids; taxanes; epothilones; antimicrotubule agents; alkylating agents; antimetabolites; topoisomerase inhibitors; aziridine-containing compounds; and other related cytotoxic and cytostatic agents.

94. The method of claim 89, which further comprises the administration of one or more additional cancer treating agents including: (i) hormones, hormonal complexes, and antihormones selected from the group comprising: interleukins, interferons, leuprolide, and pegasparaginase; (ii) enzymes, proteins, peptides, and antivirals selected from the group consisting of: acyclovir and zidovudine; (iii) cytotoxic agents, cytostatic agents; (iv) polyclonal and monoclonal antibodies; (vii) kinase inhibitors; (viii) ALK inhibitors; (ix) cancer vaccines; (x) Antibody Drug Conjugates; and/or (xi) chimeric antigen receptor T-cell (CAR-T) Therapy.

95. A method for the formation of adducts comprising the covalent-binding of one or more sulfur-containing, amino acid-specific small molecules of the present invention to one or more cysteine amino acid residues within a target molecule selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif;
wherein said adduct formation comprising the covalent-binding of one or more sulfur-containing, amino acid-specific small molecules of the present invention to one or more cysteine amino acid residues within said target molecule(s) has the ability to modify and/or modulate abnormal expression and/or biochemical activity of said target molecule(s) so as to provide a therapeutic benefit to a subject with one or more types of cellular metabolic anomalies or other undesirable physiological conditions that exhibit evidence of: (i) abnormal biochemical activity and/or (ii) abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, and other target molecules possessing a similar active site or structural motif.

96. The method of claim 94, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 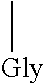
pharmaceutically-acceptable salts thereof.

97. The method of claim 95, wherein said sulfur-containing, amino acid-specific small molecule is disodium 2,2′-dithio-bis-ethane sulfonate.

98. A method for quantitatively determining the level of DNA, mRNA, and/or protein of a target molecule selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, in cells which have been isolated from a patient who has been already been diagnosed or is suspected of having a non-cancerous cellular metabolic anomaly or other undesirable physiological condition;
wherein the method used to quantitatively determine the levels of the DNA, mRNA, and/or protein of a target molecule(s) is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

99. A method for quantitatively determining the level of DNA, mRNA, and/or protein of a target molecule selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), ribonucleotide reductase, tubulin, farnesyltransferase, and other target molecules possessing a similar active site or structural motif, in cells which have been isolated from a patient who has already been diagnosed with cancer or is suspected of having cancer;
wherein the method used to quantitatively determine the levels of the DNA, mRNA, and/or protein of a target molecule(s) is selected from the group consisting of: (a) fluorescence in situ hybridization (FISH), nucleic acid microarray analysis, immunohistochemistry (IHC), radioimmunoassay (RIA), quantitative immunofluorescence and/or automated quantitative analysis (e.g., Genoptix's AQUA); (b) ELISA approaches including, but not limited to, high-throughput ELISA, InCell ELISAs, or quantitative western analyses (e.g., Licor and related systems), and related ELISA methodologies, and flow cytometry-based analyses (e.g., Affymetrix's Luminex assay and related approaches); (c) PCR coupled with MS approaches including, but not limited to, MALDI-TOF MS (e.g., Sequenom's MassARRAY system and related approaches); (d) mass spectroscopy based methods including, but not limited to, NanoLC coupled with ESI-MS (e.g., Bruker Daltonics/Eksigent Technologies system and related approaches), LC-MS, LC-MS/MS, and other MS systems designed to generate accurate-mass, high-resolution data on heterogeneous samples; and (e) isoelectric focusing, agarose/polyacrylamide gel electrophoresis, Southern blotting, Western blotting, Northern blotting, enzyme/substrate activity assay, X-ray crystallography, and other related analytic methodologies.

100. A method to potentiate the inhibition of anaplastic lymphoma kinase (ALK) by crizotinib, wherein said method is comprised of the administration of therapeutically-effective doses of crizotinib and one or more of the sulfur-containing, amino acid-specific small molecules of the present invention.

101. The method of claim 99, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 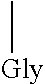
pharmaceutically-acceptable salts thereof.

102. The method of claim 100, wherein said sulfur-containing, amino acid-specific small molecule is disodium 2,2′-dithio-bis-ethane sulfonate.

103. A method to potentiate the inhibition of epidermal growth factor receptor (EGFR) by erlotinib, wherein said method is comprised of the administration of a therapeutically-effective doses of erlotinib and one or more sulfur-containing, amino acid-specific small molecules of the present invention.

104. The method of claim 102, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 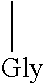
pharmaceutically-acceptable salts thereof.

105. The method of claim 103, wherein said sulfur-containing, amino acid-specific small molecule is disodium 2,2′-dithio-bis-ethane sulfonate.

106. A method for administration of a therapeutically-effective dose of one or more of the sulfur-containing, amino acid-specific small molecules of the present invention to subjects suffering from one or more types of cancer in an amount sufficient to improve the therapeutic efficacy of the cancer treating agent or agents being administered to said subject, even after cessation of treatment with said sulfur-containing, amino acid-specific small molecules to said subject.

107. A method for administration of a therapeutically-effective dose of one or more of the sulfur-containing, amino acid-specific small molecules of the present invention to subjects suffering from one or more types of cancer in an amount sufficient to make the intracellular environment of the cancer cells to be more amenable to: (i) improve responses and outcomes in subjects receiving follow-on treatment with other cancer treating agents even after cessation of treatment with such sulfur-containing, amino acid-specific small molecules of the present invention; and (ii) improve the cytotoxic performance of second-line and third-line treatment of said subject with cancer treating agents.

108. A method for increasing the 2-year survival of female non-smokers with adenocarcinoma of the lung, wherein said method is comprised of the administration of a therapeutically-effective dose of one or more of the sulfur-containing, amino acid-specific small molecules of the present invention.

109. A method for increasing the 2-year survival of females with adenocarcinoma of the lung, wherein said method is comprised of the administration of a therapeutically-effective dose of one or more of the sulfur-containing, amino acid-specific small molecules of the present invention.

110. A method for increasing the 2-year survival of male non-smokers with adenocarcinoma of the lung, wherein said method is comprised of the administration of a therapeutically-effective dose of one or more of the sulfur-containing, amino acid-specific small molecules of the present invention.

111. A method for improving biological system stability by altering the level of non-clonal chromosomal aberrations (NCCAs) in a subject having one or more types of cellular metabolic anomalies or other pathophysiological conditions, including cancer;
wherein the relative level of non-clonal chromosomal aberrations (NCCAs) is impacted by: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target proteins possessing a similar active site or structural motif; and
wherein said method is comprised of the administration of the sulfur-containing, amino acid-specific small molecules of the present invention in an amount sufficient to provide a therapeutic benefit by altering the relative level of non-clonal chromosomal aberrations (NCCAs) in the subject having one or more types of cellular metabolic anomalies or other pathophysiological conditions, including cancer.

112. A method for improving biological system stability in a subject with one or more types of cancer, where the system stability is altered by: (i) the abnormal biochemical activity and/or (ii) the abnormal expression of any combination of target molecules selected from the group consisting of:
anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target proteins possessing a similar active site or structural motif; and
wherein said method is comprised of the administration of the sulfur-containing, amino acid-specific small molecules of the present invention in an amount sufficient to provide a therapeutic benefit by altering: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target proteins possessing a similar active site or structural motif relative level of non-clonal chromosomal aberrations (NCCAs) in the subject having one or more types of cellular metabolic anomalies or other pathophysiological conditions, including cancer.

113. A method for the adjuvant treatment of a subject who has one or more types of cancer that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target proteins possessing a similar active site or structural motif;
wherein said method is comprised of the administration of the sulfur-containing, amino acid-specific small molecules of the present invention in an amount sufficient to provide a therapeutic benefit to the subject suffering from one or more types of cancer that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target proteins possessing a similar active site or structural motif.

114. A method for the neo-adjuvant treatment of a subject who has one or more types of cancer that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target proteins possessing a similar active site or structural motif;
wherein said method is comprised of the administration of the sulfur-containing, amino acid-specific small molecules of the present invention prior to the subsequent administration of the primary chemotherapeutic regimen in an amount sufficient to provide a therapeutic benefit to the subject suffering from one or more types of cancer that involve: (i) the abnormal biochemical activity and/or (ii) the abnormal expression of any combination of target molecules selected from the group consisting of: anaplastic lymphoma kinase (ALK), mesenchymal epithelial transition (MET) kinase, the receptor tyrosine kinase (ROS1), epidermal growth factor receptor (EGFR), peroxiredoxin (Prx), excision repair cross-complementing protein 1 (ERCC1), insulin growth factor 1 receptor (IGF1R), tubulin, ribonucleotide reductase (RNR), farnesyltransferase, and other target proteins possessing a similar active site or structural motif.

115. The method of any one of claims 105-113, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 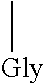
pharmaceutically-acceptable salts thereof.

116. The method of claim 114, wherein said sulfur-containing, amino acid-specific small molecule is disodium 2,2′-dithio-bis-ethane sulfonate.

117. A method for the treatment of a subject who has one or more types of cancer that involve a T790 mutation in the epidermal growth factor receptor (EGFR) gene; wherein said method is comprised of the administration of the sulfur-containing, amino acid-specific small molecules of the present invention in an amount sufficient to provide a therapeutic benefit to the subject suffering from one or more types of cancer that involve a T790 mutation in the epidermal growth factor receptor (EGFR) gene.

118. The method of claim 116, wherein said sulfur-containing, amino acid-specific small molecules are selected from the group consisting of: (i) 2,2′-dithio-bis-ethane sulfonate; (ii) the metabolite of 2,2′-dithio-bis-ethane sulfonate, known as 2-mercapto ethane sulfonate; and (iii) 2-mercapto-ethane sulfonate conjugated as a disulfide with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, -Homocysteine-Glu, and 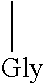
pharmaceutically-acceptable salts thereof.

119. The method of claim 117, wherein said sulfur-containing, amino acid-specific small molecule is disodium 2,2′-dithio-bis-ethane sulfonate.


SEQUENCE LISTING (76 sequences)

SEQ ID NO: 1 - protein, 5 aa, Homo sapiens
MDGPK

SEQ ID NO: 2 - protein, 5 aa, Homo sapiens
NFALR

SEQ ID NO: 3 - protein, 6 aa, Homo sapiens
GNPVLK

SEQ ID NO: 4 - protein, 6 aa, Homo sapiens
LQSLGK

SEQ ID NO: 5 - protein, 7 aa, Homo sapiens
DPQQALK

SEQ ID NO: 6 - protein, 6 aa, Homo sapiens
YLETYK

SEQ ID NO: 7 - protein, 9 aa, Homo sapiens
SNSIIVSPR

SEQ ID NO: 8 - protein, 8 aa, Homo sapiens
LEQPFVSR

SEQ ID NO: 9 - protein, 9 aa, Homo sapiens
VTECLTTVK

SEQ ID NO: 10 - protein, 9 aa, Homo sapiens
VLLVQVDVK

SEQ ID NO: 11 - protein, 12 aa, Homo sapiens
EGVPQPSGPPAR

SEQ ID NO: 12 - protein, 13 aa, Homo sapiens
EDLALCPGLGPQK

SEQ ID NO: 13 - protein, 11 aa, Homo sapiens
LFDVLHEPFLK

SEQ ID NO: 14 - protein, 12 aa, Homo sapiens
YHNLHPDYIHGR

SEQ ID NO: 15 - protein, 17 aa, Homo sapiens
AYEQKPADCTLILAWSP

SEQ ID NO: 16 - protein, 20 aa, Homo sapiens
MCILADCTLILAWSPEEAGR

SEQ ID NO: 17 - protein, 7 aa, Homo sapiens
MSSGNAK

SEQ ID NO: 18 - protein, 9 aa, Homo sapiens
TGHPAPNFK

SEQ ID NO: 19 - protein, 19 aa, Homo sapiens
ATAVMPDGQFKDISGSDYK

SEQ ID NO: 20 - protein, 2 aa, Homo sapiens
GK

SEQ ID NO: 21 - protein, 25 aa, Homo sapiens
YVVFFFYPLDFFFVCPTEIIAFSDR

SEQ ID NO: 22 - protein, 5 aa, Homo sapiens
AEEFK

SEQ ID NO: 23 - protein, 24 aa, Homo sapiens
LNCQVIGASVDSHFCHCAWVNTPK

SEQ ID NO: 24 - protein, 1 aa, Homo sapiens
K

SEQ ID NO: 25 - protein, 16 aa, Homo sapiens
QGGLGPMNIPLVSDPK

SEQ ID NO: 26 - protein, 1 aa, Homo sapiens
R

SEQ ID NO: 27 - protein, 10 aa, Homo sapiens
TIAQDYGVLK

SEQ ID NO: 28 - protein, 8 aa, Homo sapiens
ADEGISFR

SEQ ID NO: 29 - protein, 8 aa, Homo sapiens
GLFIIDDK

SEQ ID NO: 30 - protein, 4 aa, Homo sapiens
GILR

SEQ ID NO: 31 - protein, 11 aa, Homo sapiens
QITVNDLPVGR

SEQ ID NO: 32 - protein, 7 aa, Homo sapiens
SVDETLR

SEQ ID NO: 33 - protein, 10 aa, Homo sapiens
LVQAFQFTDK

SEQ ID NO: 34 - protein, 10 aa, Homo sapiens
HGEVCPAGWK

SEQ ID NO: 35 - protein, 12 aa, Homo sapiens
PGSDTIKPDVQK

SEQ ID NO: 36 - protein, 2 aa, Homo sapiens
SK

SEQ ID NO: 37 - protein, 5 aa, Homo sapiens
EYFSK

SEQ ID NO: 38 - protein, 2 aa, Homo sapiens
QK

SEQ ID NO: 39 - protein, 15 aa, Homo sapiens
MSSGNAKIGHPAPNF

SEQ ID NO: 40 - protein, 11 aa, Homo sapiens
KATAVMPDGQF

SEQ ID NO: 41 - protein, 8 aa, Homo sapiens
KDISLSDY

SEQ ID NO: 42 - protein, 4 aa, Homo sapiens
KGKY

SEQ ID NO: 43 - protein, 3 aa, Homo sapiens
VVF

SEQ ID NO: 44 - protein, 1 aa, Homo sapiens
F

SEQ ID NO: 45 - protein, 1 aa, Homo sapiens
F

SEQ ID NO: 46 - protein, 5 aa, Homo sapiens
YPLDF

SEQ ID NO: 47 - protein, 2 aa, Homo sapiens
TF

SEQ ID NO: 48 - protein, 9 aa, Homo sapiens
VCPTEIIAF

SEQ ID NO: 49 - protein, 7 aa, Homo sapiens
SDRAEEF

SEQ ID NO: 50 - protein, 13 aa, Homo sapiens
KKLNCQVIGASVD

SEQ ID NO: 51 - protein, 5 aa, Homo sapiens
CHLAW

SEQ ID NO: 52 - protein, 29 aa, Homo sapiens
VNTPKKQGGLGPMNIPLVSDPKRTIAQDY

SEQ ID NO: 53 - protein, 15 aa, Homo sapiens
GVLKADEGISFRGLF

SEQ ID NO: 54 - protein, 4 aa, Homo sapiens
RGLF

SEQ ID NO: 55 - protein, 32 aa, Homo sapiens
IIDPKGILRQITVNDLPVGRSVDETLRLVQAF

SEQ ID NO: 56 - protein, 2 aa, Homo sapiens
QF

SEQ ID NO: 57 - protein, 11 aa, Homo sapiens
TDKHGEVCPAG

SEQ ID NO: 58 - protein, 17 aa, Homo sapiens
KPGSDTIKPDVQKSKEY

SEQ ID NO: 59 - protein, 1 aa, Homo sapiens
F

SEQ ID NO: 60 - protein, 4 aa, Homo sapiens
SKQK

SEQ ID NO: 61 - protein, 9 aa, Homo sapiens
HHHHHHMVK

SEQ ID NO: 62 - protein, 5 aa, Homo sapiens
QIESK

SEQ ID NO: 63 - protein, 13 aa, Homo sapiens
TAFQEALDAAGDK

SEQ ID NO: 64 - protein, 15 aa, Homo sapiens
LVVVDFSATWCGPCK

SEQ ID NO: 65 - protein, 3 aa, Homo sapiens
MIK

SEQ ID NO: 66 - protein, 9 aa, Homo sapiens
PFFHSLSEK

SEQ ID NO: 67 - protein, 24 aa, Homo sapiens
YSNVIFIEVDVDDCQDVASECEVK

SEQ ID NO: 68 - protein, 9 aa, Homo sapiens
CMPTFQFFK

SEQ ID NO: 69 - protein, 1 aa, Homo sapiens
K

SEQ ID NO: 70 - protein, 3 aa, Homo sapiens
GQK

SEQ ID NO: 71 - protein, 9 aa, Homo sapiens
VGEFSGANK

SEQ ID NO: 72 - protein, 2 aa, Homo sapiens
EK

SEQ ID NO: 73 - protein, 9 aa, Homo sapiens
LEATINELV

SEQ ID NO: 74 - protein, 297 aa, Homo sapiens
ADPGKDKEGVPQPSGPPARKKFVIPLDEDEVPPGVAKPLFRSTQSLPTVPTSAQAAPQTYAETYAISQPLEGAGATCPTGSEPLAGETPNQALKPGAKSNSIIVSPPQRGNPVLKFVRMVPWEFGDVIPDYVLGQSTCALFLSLRYANLHPDYIHGRLQSLGKNFALRVLLVQVDKDPQQALKEKAKMCILADCTLILAWSPEEAGRYLETYKAYEQKPADLLMEKLEQDFVSRVTECLTTVKSVNKTDSQTLLTTFGSLEQLIAASREDLALCPGLFPQKARRLFDVLHEPFLKVP

SEQ ID NO: 75 - protein, 9 aa, Homo sapiens
CMPTFQFFK

SEQ ID NO: 76 - protein, 24 aa, Homo sapiens
YSNVIFLEVAVDACQDVASECEVK